(19) United States
(12) Patent Application Publication
(10) Pub. No.: US 2026/0258025 A1
(43) Pub. Date: Sep. 3, 2026

(54) INHIBITORS OF TDP-43 AND TAU AGGREGATION

(71) Applicant: Aquinnah Pharmaceuticals, inc., Cambridge, MA (US)

(72) Inventor: Joseph P. Vacca, Telford, PA (US)

(21) Appl. No.: 19/163,007

(22) PCT Filed: Mar. 8, 2024
(86) PCT No.: PCT/US2024/019162

§ 371 Date: Sep. 8, 2025

Related U.S. Application Data
(60) Provisional application No. 63/489,407, filed on Mar. 9, 2023.

(51) Int. Cl. C07D 413/14; A61K 31/4025; A61K 31/4155; A61K 31/454; A61K 31/4545; A61K 31/4709; A61K 31/4725; A61K 31/496; A61K 31/497; A61K 31/506; A61K 31/5377; A61P 25/28; C07D 409/06; C07D 409/14

(52) U.S. Cl. CPC C07D 413/14; A61K 31/4025; A61K 31/4155; A61K 31/454; A61K 31/4545; A61K 31/4709; A61K 31/4725; A61K 31/496; A61K 31/497; A61K 31/506; A61K 31/5377; A61P 25/28; C07D 409/06; C07D 409/14

(57) ABSTRACT

Herein, compounds, compositions and methods for modulating inclusion formation and stress granules in cells related to the onset of neurodegenerative diseases, musculoskeletal diseases, cancer, ophthalmological diseases, and viral infections are described.

### RELATED APPLICATIONS

This application claims the benefit of and priority to U.S. Provisional Patent Application Ser. No. 63/489,407, filed Mar. 9, 2023, the contents of which are hereby incorporated by reference.

### BACKGROUND

One of the hallmarks of many neurodegenerative diseases is the accumulation of protein inclusions in the brain and central nervous system. These inclusions are insoluble aggregates of proteins and other cellular components that cause damage to cells and result in impaired function. Proteins such as tau, α-synuclein, huntingtin and β-amyloid have all been found to form inclusions in the brain and are linked to the development of a number of neurodegenerative diseases, including Alzheimer's disease and Huntington's disease. Recently, the TDP-43 protein was identified as one of the major components of protein inclusions that typify the neurogenerative diseases Amyotrophic Lateral Sclerosis (ALS) and Frontotemporal Lobar Dementia with ubiquitin inclusions (FTLD-U). Abnormalities in TDP-43 biology appear to be sufficient to cause neurodegenerative disease, as studies have indicated that mutations in TDP-43 occur in familial ALS. In addition, TDP-43 has been found to play a role in the stress granule machinery. Analysis of the biology of the major proteins that accumulate in other neurodegenerative diseases has led to major advances in our understanding of the pathophysiology of TDP-43 inclusions as well as the development of new drug discovery platforms. Tau aggregation is also believed important in pathological processes of disease, in particular neurodegenerative disease.

Currently, it is believed that aggregates that accumulate in neurodegenerative diseases like ALS, FTLD-U, Parkinson's disease and Huntington's disease accumulate slowly and are very difficult to disaggregate or perhaps cannot be disaggregated. Thus, there is an unmet need for compositions and methods that can rapidly disaggregate these accumulating proteins, more specifically, TDP-43 and tau, or inhibit the formation of aggregates altogether.

### SUMMARY

In an aspect, the disclosure provides a compound of Formula (I):

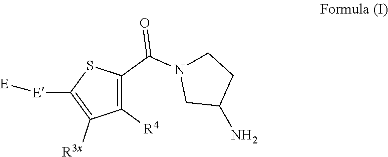

or stereoisomer and/or a pharmaceutically acceptable salt thereof, wherein:
E is C₃-C₇ cycloalkyl, heterocycloalkyl, aryl, heteroaryl, carbocyclyl, or heterocyclyl, and E may be optionally substituted;
E′ is absent, or E′ is C₃-C₇ cycloalkyl, heterocycloalkyl, aryl, heteroaryl, carbocyclyl, or heterocyclyl, and E′ may be optionally substituted; and
R^{3x} and R⁴ are each H or an independently selected optional substituent.

In some embodiments, E′ is absent.

In another aspect, the disclosure provides a compound of Formula (II):

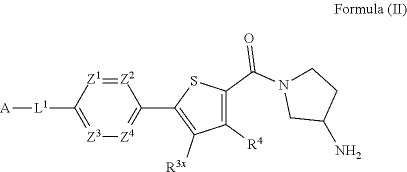

or stereoisomer and/or a pharmaceutically acceptable salt thereof, wherein:
Z¹ is N or CR¹¹;
Z² is N or CR¹²;
Z³ is N or CR¹³;
Z⁴ is N or CR¹⁴;
L¹ is absent, or L¹ is C₁-C₆ alkylene, C₁-C₆ heteroalkylene, —O—, —S—, or —NR′—, wherein the C₁-C₆ alkylene and C₁-C₆ heteroalkylene are optionally substituted;
A is H, halo, C₁-C₆ alkyl, C₁-C₆ heteroalkyl, C₁-C₆ haloalkyl, C₃-C₇ cycloalkyl, heterocycloalkyl, aryl, heteroaryl, carbocyclyl, or heterocyclyl, and A may be optionally substituted; or A and R¹¹ are taken together with the atoms to which they are attached to form an optionally substituted cycloalkyl, heterocycloalkyl, aryl, heteroaryl, carbocyclic, or heterocyclic ring;
R¹¹, R¹², R¹³, and R¹⁴ are each independently H or an optional substituent;
each R′ is H or C₁-C₆ alkyl; and
each of R^{3x} and R⁴ is independently H or an independently selected optional substituent,
wherein no more than two of Z¹, Z², Z³ and Z⁴ are N.

In another aspect, the disclosure provides a compound of Formula (II):

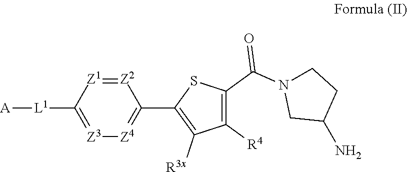

or stereoisomer and/or a pharmaceutically acceptable salt thereof, wherein:
Z¹ is N or CR¹¹;
Z² is N or CR¹²;
Z³ is N or CR¹³;
Z⁴ is N or CR¹⁴;
L¹ is absent, or L¹ is C₁-C₆ alkylene, C₁-C₆ heteroalkylene, —O—, —S—, or —NR′—, wherein the C₁-C₆ alkylene and C₁-C₆ heteroalkylene are optionally substituted by 1-4 independently substituents selected from ═O (oxo), OH, and halogen;
A is H, halo, C₁-C₁₀ alkyl, C₁-C₁₀ heteroalkyl, C₁-C₆ haloalkyl, C₃-C₇ cycloalkyl, 3-7 membered heterocycloalkyl, C₆-C₁₀ aryl, 5-6 membered heteroaryl, C₆-C₁₀ carbocyclyl, or -5-10 membered heterocyclic ring, and A is substituted by one R¹ group and optionally substituted by 1-4 independently selected R² groups; or A and R¹¹ are taken together with the atoms to which they are attached to form a C₃-C₇ cycloalkyl, aryl, 5-6 membered heteroaryl, or 3-7 membered heterocyclic ring, wherein the heteroaryl and heterocyclyl have 1-3 ring heteroatoms selected from N, O, and S, and wherein each of the rings is optionally substituted by one R¹ group and optionally substituted by 1-4 independently selected R² groups;
each of R¹¹, R¹², R¹³, and R¹⁴ is independently H or R³;
R¹ is H, C₁-C₆ alkyl, C₂-C₆ alkenyl, C₂-C₆ alkynyl, C₂-C₉ heteroalkynyl, C₁-C₆ heteroalkyl, C₁-C₆ haloalkyl, —NH₂, or -L²-G, wherein the C₁-C₆ alkyl may be optionally substituted with OH, and wherein the C₁-C₆ heteroalkyl may be optionally substituted with C₂-C₆ heteroalkynyl;
L² is absent, or L² is C₁-C₆ alkylene, C₁-C₆ heteroalkylene, —O—, —S—, or —NR′—, wherein alkylene and heteroalkylene are optionally substituted by 1-4 substituents independently selected from ═O (oxo), OH, and halogen;
G is C₃-C₇ cycloalkyl, 3-7 membered heterocycloalkyl, aryl, 5-6 membered heteroaryl, 6-10 membered carbocyclyl, or 5-10 membered heterocyclyl, wherein heterocycloalkyl, heteroaryl, and heterocyclyl have 1-3 ring heteroatoms selected from N, O, and S, and wherein G may be optionally substituted by 1-4 R^{A} substituents;
each R^{A} is independently selected from the group consisting of C₁-C₆ alkyl, C₁-C₆ heteroalkyl, C₁-C₆ alkoxy, C₁-C₆ haloalkyl, C₁-C₆ haloalkoxy, ═O (oxo), OH, —NMe₂, —NHMe, —NH₂, CN, and halo;
each R² is independently selected from the group consisting of C₁-C₆ alkyl, C₂-C₆ alkenyl, C₂-C₆ alkynyl, C₁-C₆ heteroalkyl, C₁-C₆ alkoxy, C₁-C₆ haloalkyl, C₁-C₆ haloalkoxy, ═O (oxo), —OH, —NMe₂, —NHMe, —NH₂, and halo;
each R³ is independently selected from the group consisting of C₁-C₆ alkyl, C₂-C₆ alkenyl, C₂-C₆ alkynyl, C₁-C₆ alkoxy, C₁-C₆ haloalkyl, C₁-C₆ haloalkoxy, OH, cyano, and halo;
R⁴ is selected from the group consisting of H, C₁-C₆ alkyl, C₂-C₆ alkenyl, C₂-C₆ alkynyl, C₁-C₆ heteroalkyl, C₁-C₆ haloalkyl, C₁-C₆ haloalkoxy, cyano, and halo;
each R′ is H or C₁-C₆ alkyl; and
R^{3x} is selected from the group consisting of H, C₁-C₆ alkyl, C₂-C₆ alkenyl, C₂-C₆ alkynyl, C₁-C₆ alkoxy, C₁-C₆ haloalkyl, cyano, and halo, wherein no more than two of Z¹, Z², Z³ and Z⁴ are N.

In some embodiments, R^{3x} is H or C₁-C₆ alkyl.

In some embodiments, R^{3x} is H or -Me.

In some embodiments, R⁴ is H, halo, C₁-C₆ haloalkoxy, C₁-C₆ alkyl, C₁-C₆ alkoxy, C₂-C₆ alkenyl, or C₁-C₆ heteroalkyl.

In some embodiments, R⁴ is H, —Cl, —Br, —OCF₃, -Me, —OMe, -Et, -nPr, -iPr,

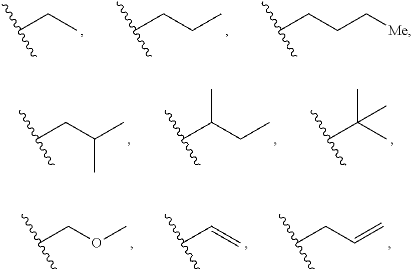

—CH₂OCH₃, —CH═CH₂, or —CH₂CH═CH₂.

In some embodiments, Z¹ is CR¹¹, Z² is CR¹², Z³ is CR¹³, and Z⁴ is CR¹⁴.

In some embodiments, Z¹ is N, Z² is CR¹², Z³ is CR¹³, and Z⁴ is CR¹⁴.

In some embodiments, Z¹ is CR¹¹, Z² is N, Z³ is CR¹³, and Z⁴ is CR¹⁴.

In some embodiments, Z¹ is N, Z² is CR¹², Z³ is N, and Z⁴ is CR¹⁴.

In some embodiments, Z¹ is N, Z² is N, Z³ is CR¹³, and Z⁴ is CR¹⁴.

In some embodiments, Z¹ is CR¹¹, Z² is N, Z³ is CR¹³, and Z⁴ is N.

In some embodiments, each of R¹¹, R¹², R¹³, and R¹⁴ is independently H or R³ wherein each R³ is independently selected from the group consisting of C₁-C₆ alkyl, C₁-C₆ alkoxy, C₁-C₆ haloalkyl, C₁-C₆ haloalkoxy, OH, and halo.

In some embodiments, each of R¹¹, R¹², R¹³, and R¹⁴ is independently H or R³ wherein each R³ is independently selected from the group consisting of -Me, -Et, -nPr, -iPr, —CF₃, —OMe, —OCF₃, —OH, —F, and —Cl.

In some embodiments, L¹ is absent.

In some embodiments, L¹ is C₁-C₆ alkylene, C₁-C₆ heteroalkylene, or —O—.

In some embodiments, L¹ is —CH₂—, —OCH₂—, —NHCH₂—, —N(CH₃)CH₂—, or —O—.

In some embodiments, A is selected from the group consisting of halo, C₁-C₁₀ alkyl, and C₁-C₁₀ heteroalkyl, wherein the C₁-C₁₀ alkyl and C₁-C₁₀ heteroalkyl are optionally substituted by ═O (oxo).

In some embodiments, A is selected from the group consisting of —F, —Cl, -Me, -Et, -nPr, -iPr,

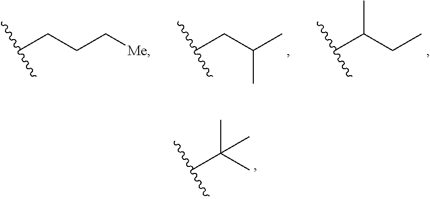

—NHCH₃, —N(CH₃)₂,

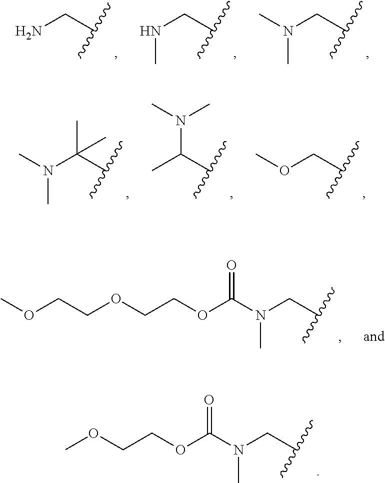

In some embodiments, A is selected from the group consisting of C₃-C₇ cycloalkyl, 3-7 membered heterocycloalkyl, C₆-C₁₀ aryl, 5-6 membered heteroaryl, C₆-C₁₀ carbocyclyl, and 5-10 membered heterocyclic ring, and A is substituted by one R¹ group and optionally substituted by 1-4 independently selected R² groups.

In some embodiments, A is selected from the group consisting of C₃-C₇ cycloalkyl, 3-7 membered heterocycloalkyl, C₆-C₁₀ aryl, and 5-6 membered heteroaryl, and A is substituted by one R group and optionally substituted by 1-4 independently selected R² groups.

In some embodiments, A is selected from the group consisting of

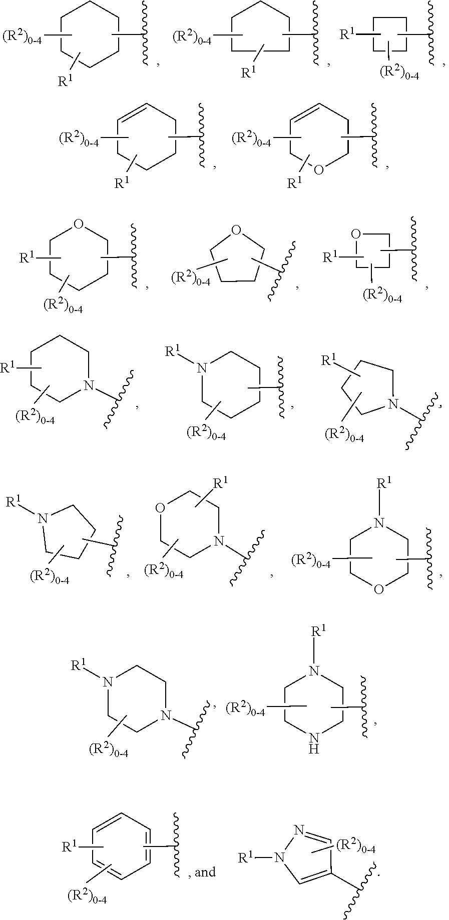

In some embodiments, each R² is independently selected from the group consisting of C₁-C₆ alkyl, C₁-C₆ alkoxy, C₁-C₆ haloalkyl, C₁-C₆ haloalkoxy, ═O (oxo), —OH, —NMe₂, —NHMe, —NH₂, and halo.

In some embodiments, each R² is independently selected from the group consisting of -Me, -Et, -nPr, -iPr, —CF₃, —OMe, —OCF₃, ═O (oxo), —OH, —NMe₂, —NHMe, —NH₂, F, Cl, and Br.

In some embodiments, R¹ is H or -L²-G.

In some embodiments, R¹ is -L²-G.

In some embodiments, L² is absent.

In some embodiments, L² is selected from the group consisting of C₁-C₆ alkylene, C₁-C₆ heteroalkylene, and —O—.

In some embodiments, L² is —CH₂—.

In some embodiments, G is C₃-C₇ cycloalkyl or 3-7 membered heterocycloalkyl, wherein the 3-7 membered heterocycloalkyl has 1-3 ring heteroatoms selected from N, O, and S, and wherein G may be optionally substituted by 1-4 R^{A} substituents.

In some embodiments, G is 3-7 membered heterocycloalkyl, wherein G may be optionally substituted by 1-4 R^{A} substituents.

In some embodiments, G is selected from the group consisting of

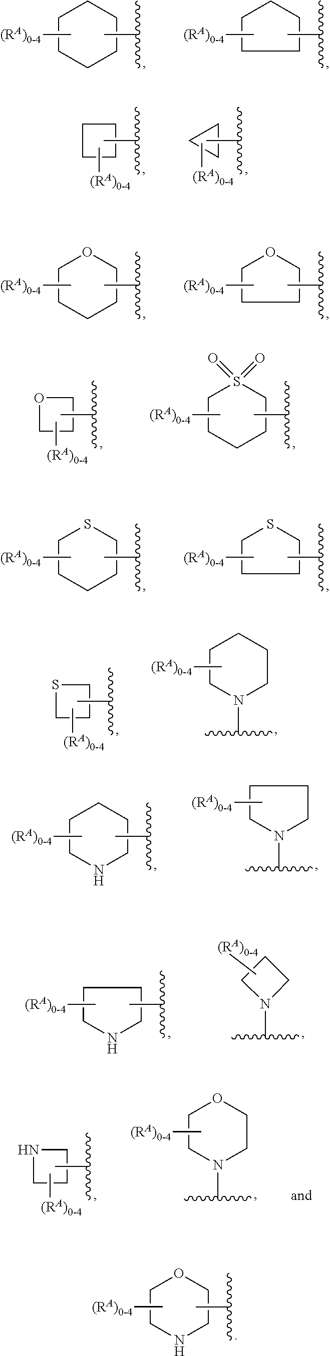

In some embodiments, each R^{A} is C₁-C₆ alkyl.

In some embodiments, G is selected from the group consisting of

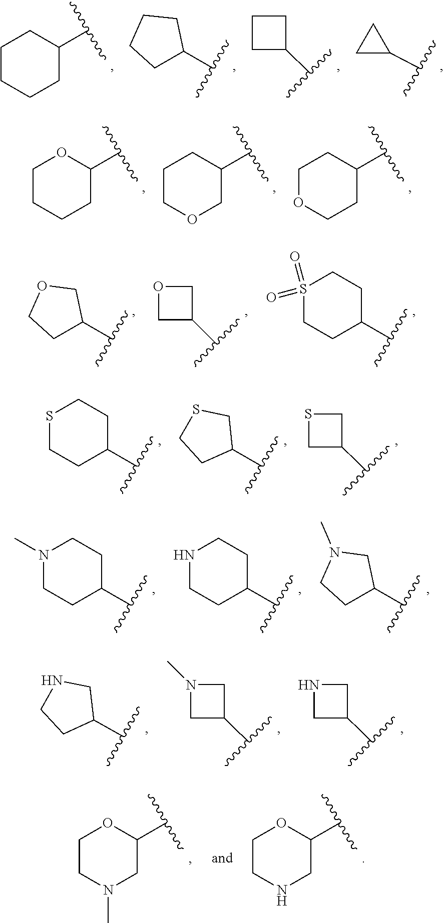

In some embodiments, A is selected from the group consisting of

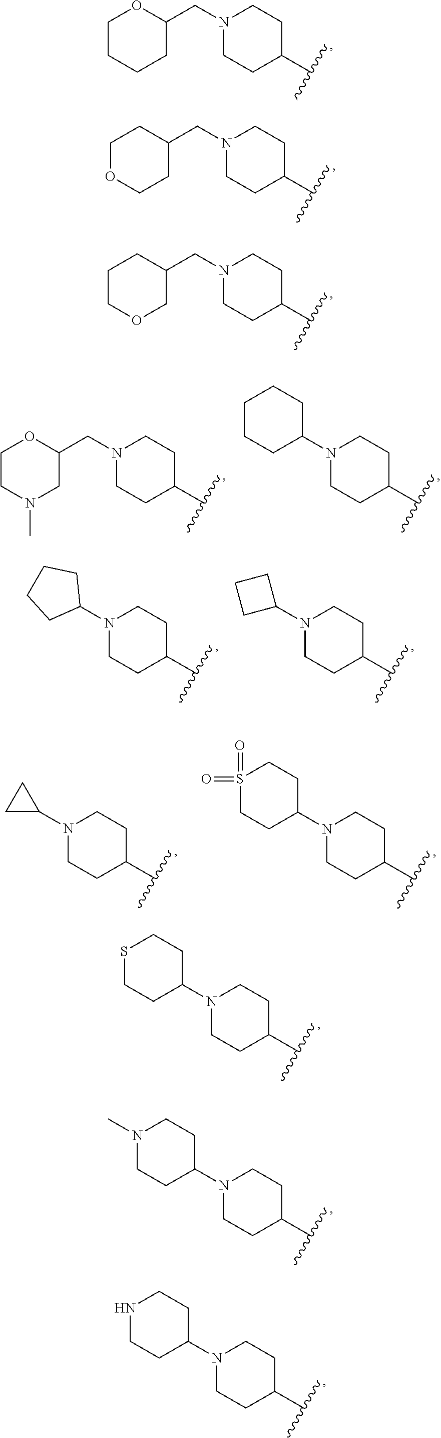

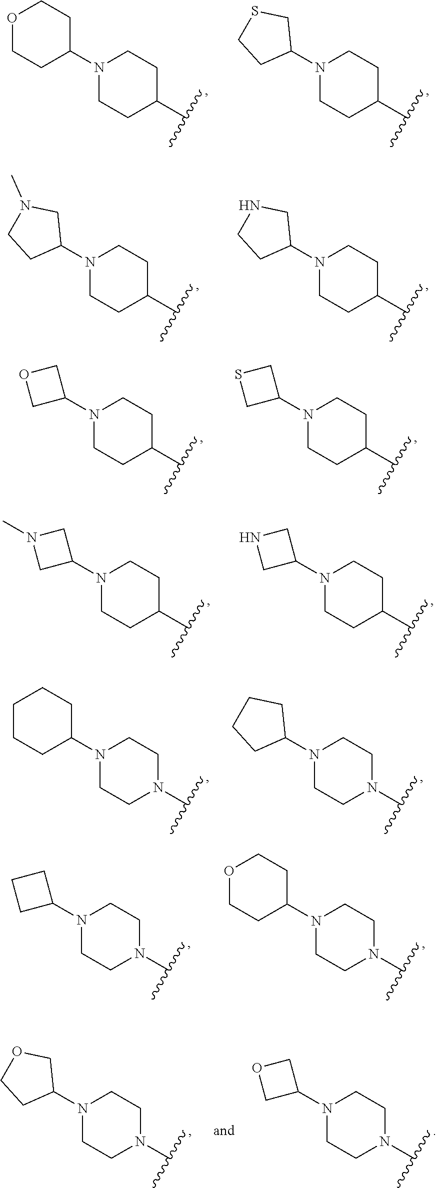

In some embodiments, A is selected from the group consisting of

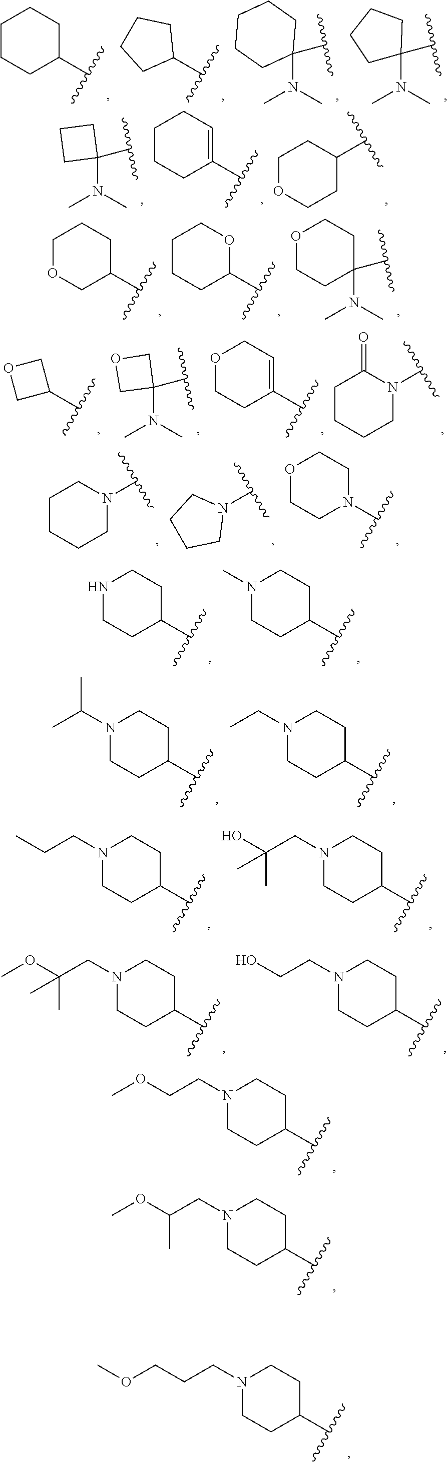

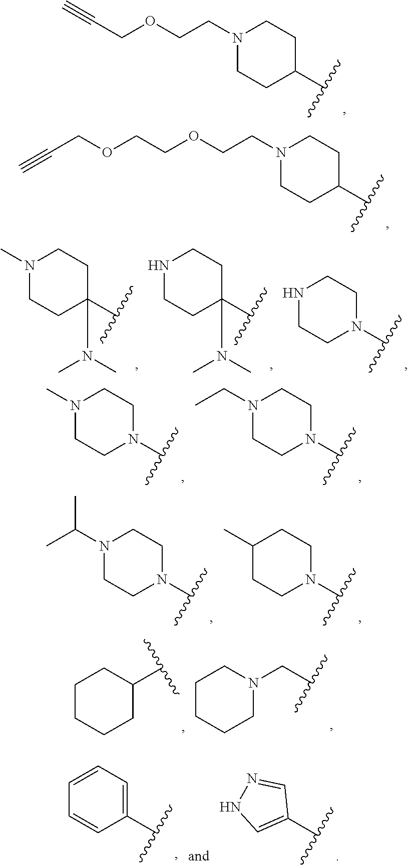

In some embodiments, A and R¹¹ are taken together with the atoms to which they are attached to form a C₃-C₇ cycloalkyl, aryl, 5-6 membered heteroaryl, or 3-7 membered heterocyclic ring, wherein the heteroaryl and heterocyclyl have 1-3 ring heteroatoms selected from N, O, and S, each of the rings optionally substituted by one R¹ group and optionally substituted by 1-4 independently selected R² groups.

In some embodiments,

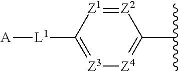

is selected from the group consisting of

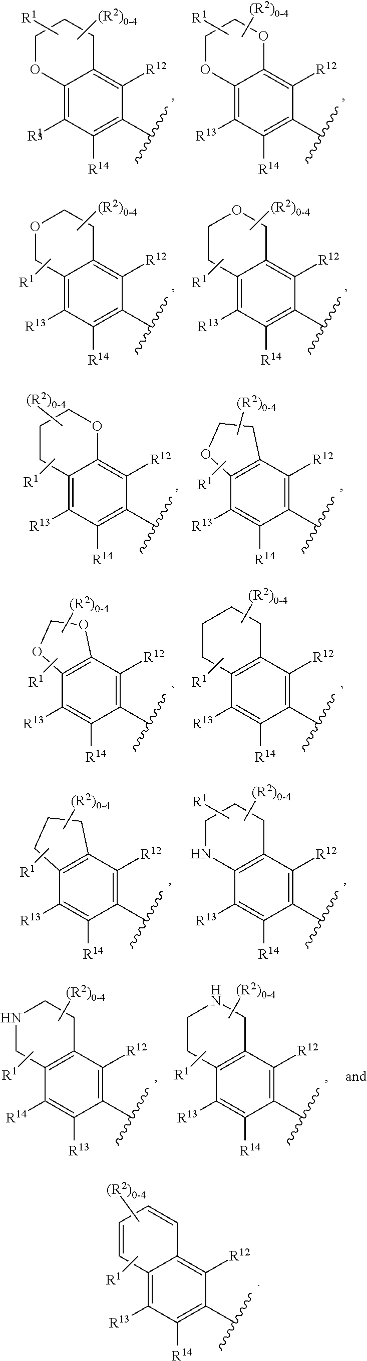

In some embodiments,

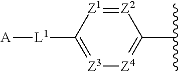

is selected from the group consisting of

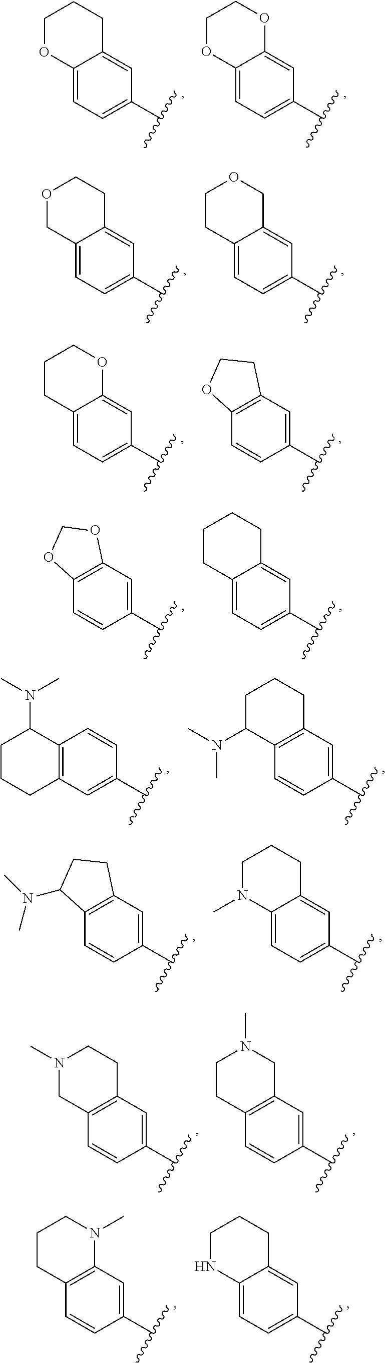

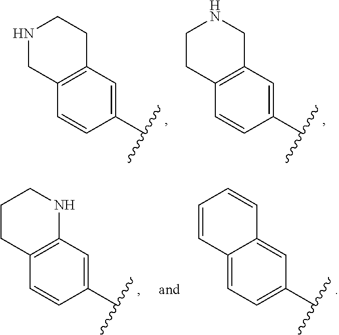 [SMILES: *c1ccc2c(c1)CCNC2 ; *c1ccc2c(c1)CNCC2 ; *c1ccc2c(c1)NCCC2 ; *c1ccc2ccccc2c1]

In some embodiments, the compound is of Formula (IIa):

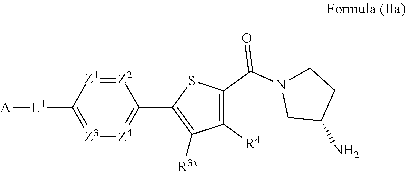

or stereoisomer and/or a pharmaceutically acceptable salt thereof.

In some embodiments, the compound is of Formula (IIb):

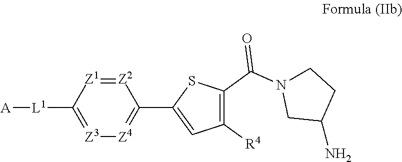

or stereoisomer and/or a pharmaceutically acceptable salt thereof.

In some embodiments, the compound is of Formula (IIc):

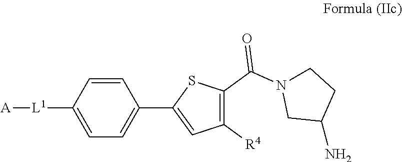

or stereoisomer and/or a pharmaceutically acceptable salt thereof.

In some embodiments, the compound is of Formula (IId):

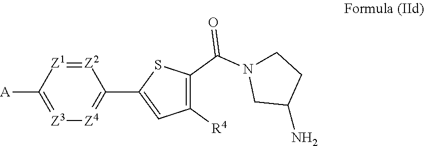

or stereoisomer and/or a pharmaceutically acceptable salt thereof.

In another aspect, the disclosure provides a compound of Formula (III):

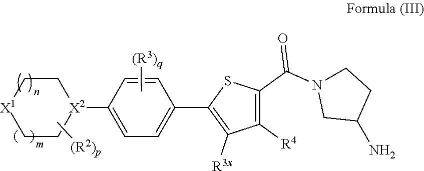

or stereoisomer and/or a pharmaceutically acceptable salt thereof, wherein:
X¹ is NR′, O, S, SO₂, CH₂ or CHR¹;
X² is N or CH;
R¹ is selected from the group consisting of H, C₁-C₆ alkyl, C₂-C₆ alkenyl, C₂-C₆ alkynyl, C₁-C₆ heteroalkyl, C₁-C₆ haloalkyl, —NH₂, and -L²-G, wherein the C₁-C₆ alkyl may be optionally substituted with one or more OH, and wherein the C₁-C₆ heteroalkyl may be optionally substituted with C₂-C₆ heteroalkynyl;
L² is absent, or L² is selected from the group consisting of C₁-C₆ alkylene, C₁-C₆ heteroalkylene, —O—, —S—, and —NR′—, wherein the C₁-C₆ alkylene and C₁-C₆ heteroalkylene are optionally substituted by 1-4 substituents independently selected from the group consisting of ═O (oxo), OH, and halogen;
G is selected from the group consisting of C₃-C₇ cycloalkyl, 3-7 membered heterocycloalkyl, aryl, 5-6 membered heteroaryl, 6-10 membered carbocyclyl, and 5-10 membered heterocyclyl, wherein the 3-7 membered heterocycloalkyl, 5-6 membered heteroaryl, and 5-10 membered heterocyclyl have 1-3 ring heteroatoms selected from N, O, and S, and wherein G may be optionally substituted by 1-4 R^{A} substituents;
each R^{A} is independently selected from the group consisting of C₁-C₆ alkyl, C₁-C₆ heteroalkyl, C₁-C₆ haloalkyl, C₁-C₆ haloalkoxy, OH, CN, and halo;
each R² is independently selected from the group consisting of C₁-C₆ alkyl, C₂-C₆ alkenyl, C₂-C₆ alkynyl, C₁-C₆ heteroalkyl, C₁-C₆ alkoxy, C₁-C₆ haloalkyl, C₁-C₆ haloalkoxy, —NH₂, and halo;
each R³ is independently selected from the group consisting of C₁-C₆ alkyl, C₂-C₆ alkenyl, C₂-C₆ alkynyl, C₁-C₆ alkoxy, C₁-C₆ haloalkyl, C₁-C₆ haloalkoxy, OH, cyano, and halo;
R^{3x} is selected from the group consisting of H, C₁-C₆ alkyl, C₂-C₆ alkenyl, C₂-C₆ alkynyl, C₁-C₆ alkoxy, C₁-C₆ haloalkyl, cyano, and halo;
R⁴ is selected from the group consisting of H, C₁-C₆ alkyl, C₂-C₆ alkenyl, C₂-C₆ alkynyl, C₂-C₉ heteroalkynyl, C₁-C₆ heteroalkyl, C₁-C₆ haloalkyl, C₁-C₆ haloalkoxy, cyano, and halo;
each R′ is H or C₁-C₆ alkyl;
m is 0 or 1;
n is 0 or 1;
p is 0, 1, 2, 3, or 4; and
q is 0, 1, 2, 3, or 4.

In another aspect, the disclosure provides a compound of Formula (III):

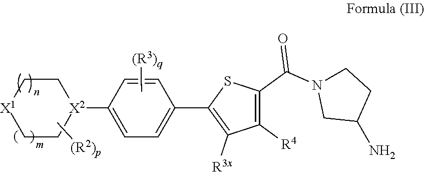

or stereoisomer and/or a pharmaceutically acceptable salt thereof, wherein:
X¹ is selected from the group consisting of NR¹, O, S, SO₂, CH₂, and CHR¹;
X² is N or CH;
R¹ is selected from the group consisting of H, C₁-C₆ alkyl, C₂-C₆ alkenyl, C₂-C₆ alkynyl, C₁-C₆ heteroalkyl, C₁-C₆ haloalkyl, —NH₂, and -G;
G is selected from the group consisting of C₃-C₇ cycloalkyl, 3-7 membered heterocycloalkyl, aryl, 5-6 membered heteroaryl, 6-10 membered carbocyclyl, and 5-10 membered heterocyclyl, wherein heterocycloalkyl, heteroaryl, and heterocyclyl have 1-3 ring heteroatoms selected from N, O, and S, and wherein G may be optionally substituted by 1-4 R^{A} substituents;
each R^{A} is independently selected from the group consisting of C₁-C₆ alkyl, C₁-C₆ heteroalkyl, C₁-C₆ haloalkyl, C₁-C₆ haloalkoxy, OH, CN, and halo;
each R² is independently selected from the group consisting of C₁-C₆ alkyl, C₁-C₆ haloalkyl, and halo;
each R³ is independently selected from the group consisting of C₁-C₆ alkyl, C₁-C₆ alkoxy, C₁-C₆ haloalkyl, cyano, and halo;
R^{3x} is selected from the group consisting of H, C₁-C₆ alkyl, C₂-C₆ alkenyl, C₂-C₆ alkynyl, C₁-C₆ alkoxy, C₁-C₆ haloalkyl, cyano, and halo;
R⁴ is selected from the group consisting of H, C₁-C₆ alkyl, C₁-C₆ alkoxy, C₁-C₆ haloalkyl, cyano, and halo;
mis 0 or 1;
nis 0 or 1;
p is 0, 1, 2, 3, or 4; and
q is 0, 1, 2, 3, or 4.

In some embodiments, the compound is of Formula (IIIa):

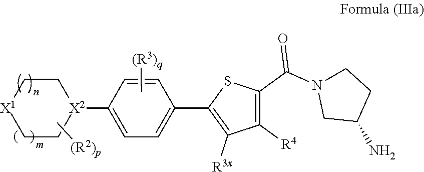

or stereoisomer and/or a pharmaceutically acceptable salt thereof.

In some embodiments, X¹ is NR¹ and X² is CH.

In some embodiments, X¹ is CH₂ or CHR¹ and X² is N.

In some embodiments, X¹ is NR¹ and X² is N.

In some embodiments, R¹ is selected from the group consisting of C₁-C₆ alkyl, C₁-C₆ alkoxy, C₁-C₆ haloalkyl, OH, and halo.

In some embodiments, R¹ is selected from the group consisting of -Me, -Et, -nPr, -iPr, —OMe, —OCF₃, —OH, —Cl, —F,

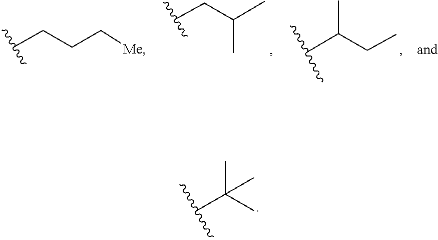

In some embodiments, R^{3x} is H or -Me.

In some embodiments, R^{3x} is H.

In some embodiments, R⁴ is -Me.

In some embodiments, m is 1 and n is 1.

In some embodiments, m is 0 and n is 1.

In some embodiments, p is 0.

In some embodiments, q is 0.

In some embodiments, the compound is of Formula (IIIb):

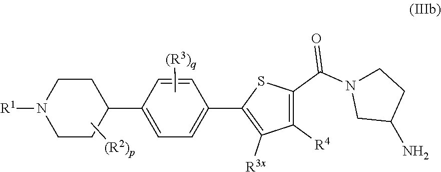

or stereoisomer and/or a pharmaceutically acceptable salt thereof.

In some embodiments, the compound is of Formula (IIIc):

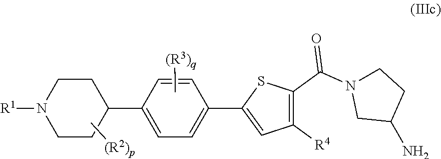

or stereoisomer and/or a pharmaceutically acceptable salt thereof

In some embodiments, the compound is of Formula (IIId):

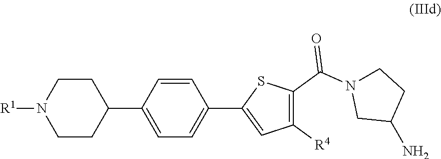

or stereoisomer and/or a pharmaceutically acceptable salt thereof

In some embodiments, the compound is of Formula (IIIe):

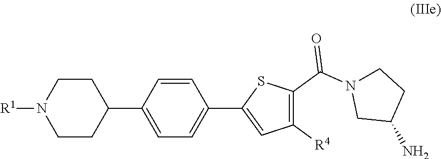

or stereoisomer and/or a pharmaceutically acceptable salt thereof.

In another aspect, the disclosure provides a compound of Formula (IV):

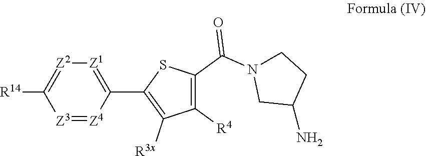

or stereoisomer and/or stereoisomer and/or a pharmaceutically acceptable salt thereof, wherein:
Z¹ is N or CR¹¹;
Z² is N or CR¹²;
Z³ is N or CR¹³;
Z⁴ is N or CH;
R¹¹ is selected from the group consisting of H, —OH, halo, C₁-C₆ alkyl, C₁-C₆ heteroalkyl, and C₁-C₆ haloalkoxy;
R¹² is selected from the group consisting of H, —OH, halo, C₁-C₆ alkyl, C₁-C₆ heteroalkyl, and C₁-C₆ haloalkoxy;
R¹³ is H or halo;
R¹⁴ is selected from the group consisting of H, halo, C₁-C₆ alkylene, C₁-C₆ heteroalkylene, 5-7 membered aryl, 5-7 membered heteroaryl, and 3-7 membered heterocycloalkyl, wherein the R¹⁴ may be optionally substituted with one or more R^{14a};
R^{14a} is selected from the group consisting of C₁-C₆ alkyl, C₁-C₆ heteroalkyl, C₃-C₇ cycloalkyl, 5-7 membered aryl, 3-7 membered heterocycloalkyl, and —C(O)OR^{14b}, wherein the C₁-C₆ alkyl or the 3-7 membered heterocycloalkyl may be optionally substituted with one or more R^{14b};
R^{14b} is selected from the group consisting of —OH, oxo, C₁-C₆ alkyl, C₁-C₈ heteroalkyl, C₂-C₆ alkynyl, and 3-7 membered heterocycloalkyl, wherein the 3-7 membered heterocycloalkyl may be optionally substituted with one or more C₁-C₆ alkyl;
wherein when Z² is CR¹², R¹² and R¹⁴ may be taken together with the atoms to which they are attached to form a cycloalkyl, heterocycloalkyl, or aryl, wherein the cycloalkyl or heterocycloalkyl may be optionally substituted with one or more C₁-C₆ alkyl or C₁-C₆ heteroalkyl;
R^{3x} is H or C₁-C₆ alkyl; and
R⁴ is selected from the group consisting of H, halo, C₁-C₆ alkyl, C₁-C₆ heteroalkyl, C₂-C₆ alkenyl, and C₁-C₆ haloalkoxy.

In some embodiments, Z¹ is CR¹¹, Z² is CR¹², Z³ is CR¹³, and Z⁴ is CH.

In some embodiments, when Z¹ is N, Z² is CR¹², Z³ is CR¹³, and Z⁴ is CH.

In some embodiments, when Z² is N, Z¹ is CR¹¹, Z³ is CR¹³, and Z⁴ is CH.

In some embodiments, when Z² and Z³ are N, Z¹ is CR¹¹ and Z⁴ is CH.

In some embodiments, when Z² and Z⁴ are N, Z¹ is CR¹¹ and Z³ is CR¹³.

In some embodiments, R¹¹ and R¹² are selected from the group consisting of halo, C₁-C₆ alkyl, C₁-C₆ heteroalkyl, and C₁-C₆ haloalkoxy.

In some embodiments, R¹¹ and R¹² are selected from the group consisting of -Me, -Et, —Cl, —F, —OMe, and —OCF₃.

In some embodiments, R¹¹ or R¹² is —OH.

In some embodiments, R¹³ is halo.

In some embodiments, R¹³ is —Cl.

In some embodiments, R¹⁴ is selected from the group consisting of H, halo, C₁-C₃ alkylene, C₁-C₆ heteroalkylene, phenyl, 5 membered heteroaryl, and 4-6 membered heterocycloalkyl, wherein the C₁-C₃ alkylene, C₁-C₆ heteroalkylene, phenyl, 5 membered heteroaryl, and 4-6 membered heterocycloalkyl may be optionally substituted with one or more R^{14a}.

In some embodiments, R¹⁴ is selected from the group consisting of ethyl, —O—, —CH₂—, -nPr, -iPr, —Cl, NHCH₃, —N(CH₃)₂,

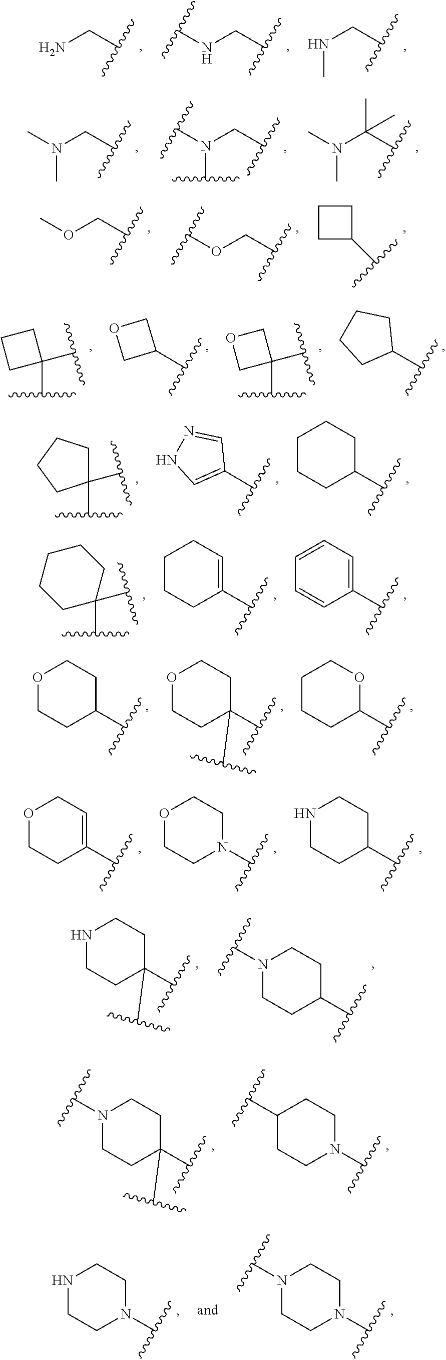

wherein if R¹⁴ contains a substitutable atom, that atom may be optionally substituted with one or more R^{14a}.

In some embodiments, R¹⁴ is selected from the group consisting of -Et, -nPr, -iPr, —Cl, NHCH₃, —N(CH₃)₂,

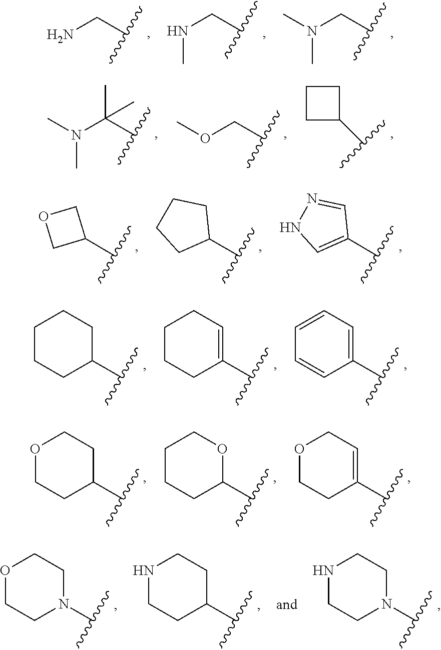

wherein if R¹⁴ contains a substitutable atom, that atom may be optionally substituted with one or more R^{14a}.

In some embodiments, R¹⁴ is selected from the group consisting of

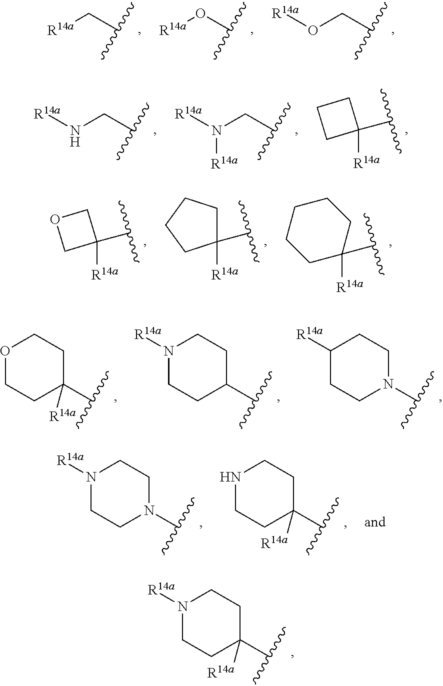

In some embodiments, R^{14a} is selected from the group consisting of C₁-C₃ alkyl, C₁-C₆ heteroalkyl, C₃-C₆ cycloalkyl, 6 membered aryl, and 4-6 membered heterocycloalkyl, wherein C₁-C₆ heteroalkyl, C₃-C₇ cycloalkyl, and 5-7 membered aryl may be optionally substituted with R^{14b}.

In some embodiments, R^{14a} is selected from the group consisting of -Me, -Et, —CH₂—, nPr,

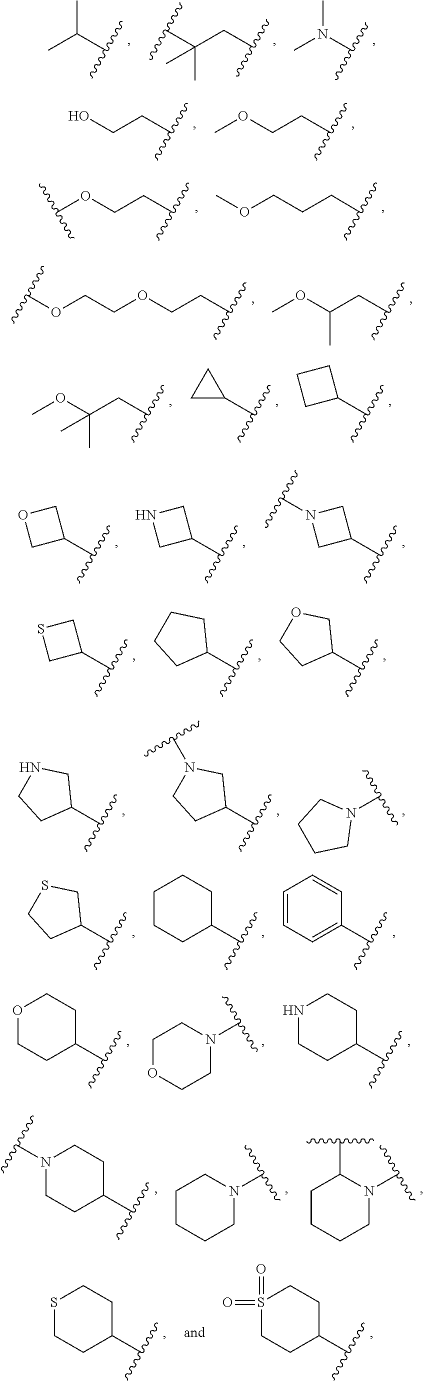

wherein if R^{14a} contains a substitutable atom, that atom may be optionally substituted with one or more R^{14b}.

In some embodiments, R^{14a} is selected from the group consisting of -Me, -Et, nPr,

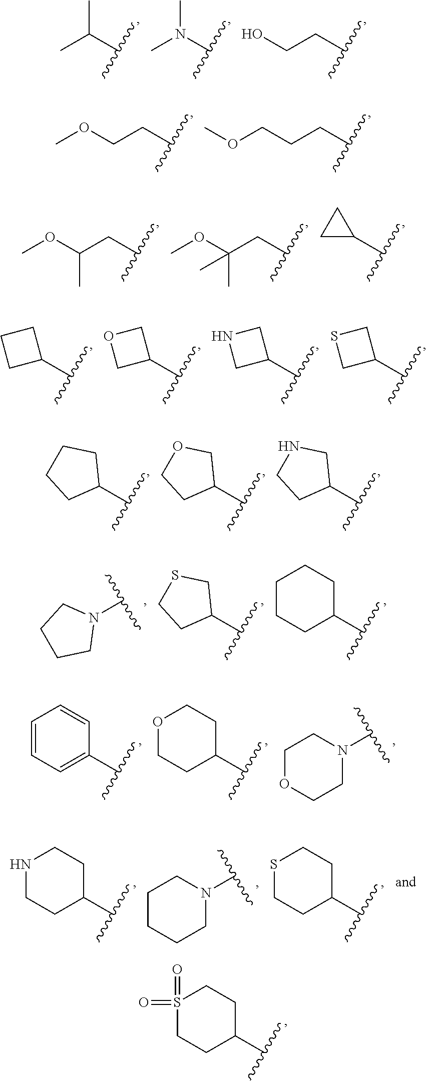

wherein if R^{14a} contains a substitutable atom, that atom may be optionally substituted with one or more R^{14b}.

In some embodiments, R^{14a} is selected from the group consisting of —C(O)OR^{14b},

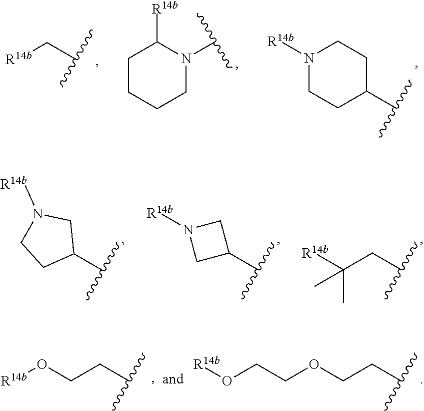

In some embodiments, R^{14b} is selected from the group consisting of oxo, —OH, -Me, C₃-C₇ heteroalkyl, C₂-C₃ alkynyl, and 6 membered heterocycloalkyl, wherein the 3-7 membered heterocycloalkyl may be optionally substituted with one or more C₁-C₆ alkyl.

In some embodiments, R^{14b} is selected from the group consisting of oxo, —OH, -Me,

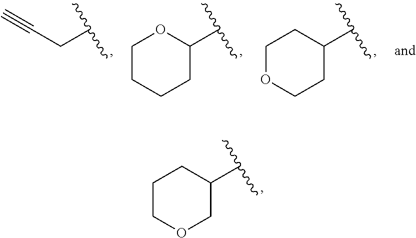

wherein the

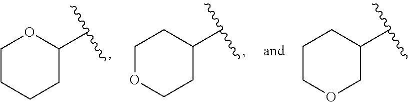 [SMILES: *C1CCCCO1 ; *C1CCOCC1 ; *C1CCCOC1]

may be optionally substituted with oxo or C₁-C₆ alkyl.

In some embodiments, R^{14b} is selected from the group consisting of oxo, —OH, -Me,

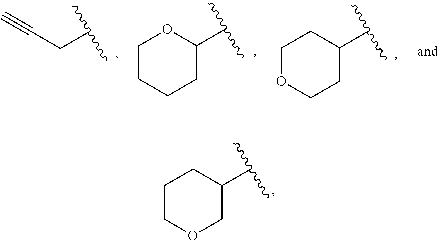

wherein the

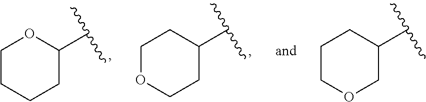 [SMILES: *C1CCCCO1 ; *C1CCOCC1 ; *C1CCCOC1]

may be optionally substituted with -Me.

In some embodiments, when Z² is CR¹², R¹² and R¹⁴ may be taken together with the atoms to which they are attached to form an aryl, cycloalkyl, or heterocycloalkyl, wherein the cycloalkyl or heterocycloalkyl may be optionally substituted with one or more C₁-C₆ alkyl or C₁-C₆ heteroalkyl.

In some embodiments, when Z² is CR¹², R¹² and R¹⁴ taken together are selected from the group consisting of

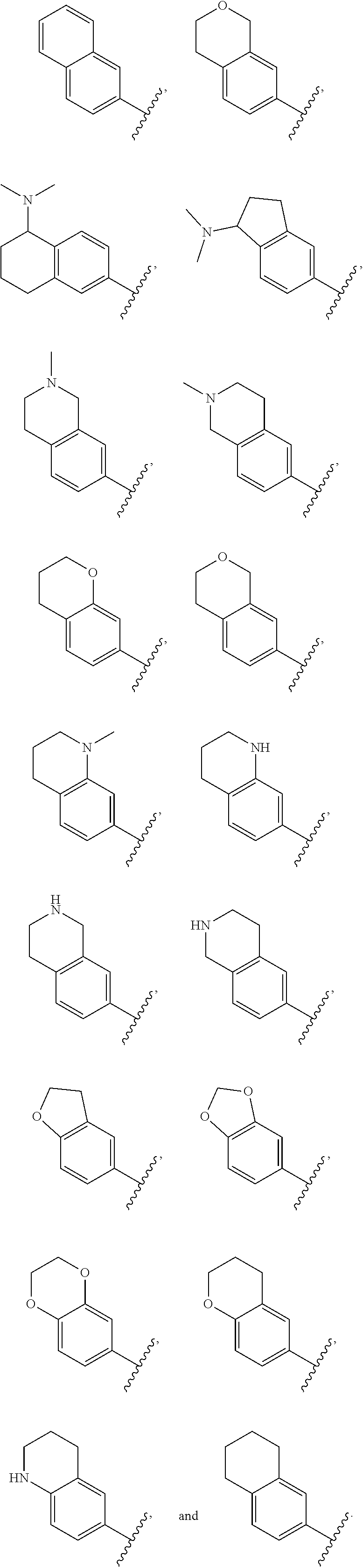

In some embodiments, R^{3x} is C₁-C₆ alkyl.

In some embodiments, R^{3x} is -Me.

In some embodiments, R⁴ is selected from the group consisting of halo, C₁-C₆ alkyl, C₁-C₆ heteroalkyl, C₂-C₆ alkenyl, and C₁-C₆ haloalkoxy.

In some embodiments, R⁴ is selected from the group consisting of —Cl, —Br, —OCF₃, -Me, -Et, —OMe,

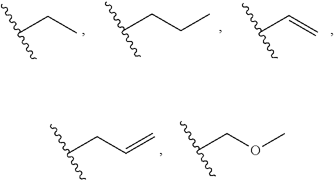

In another aspect is a compound selected from any compound set forth in Table 1, or a pharmaceutically acceptable salt thereof.

In another aspect, the disclosure provides a pharmaceutical composition comprising a pharmaceutically acceptable carrier and a compound of any one of the previous embodiments.

In another aspect is a method of treating a subject with a neurodegenerative disease or disorder, wherein the method comprises administering to a subject in need thereof a pharmaceutically effective amount of a pharmaceutical composition or a compound of any one of the previous embodiments.

In some embodiments, the neurodegenerative disease is selected from the group consisting of Alzheimer's disease, frontotemporal dementia (FTD), FTLD-U, FTD caused by mutations in the progranulin protein or tau protein (e.g., progranulin-deficient FTLD), frontotemporal dementia with inclusion body myopathy (IBMPFD), frontotemporal dementia with motor neuron disease, amyotrophic lateral sclerosis (ALS), Huntington's disease (HD), Huntington's chorea, prion diseases (e.g., Creutzfeld-Jacob disease, bovine spongiform encephalopathy, Kuru, or scrapie), Lewy Body disease, diffuse Lewy body disease (DLBD), polyglutamine (polyQ)-repeat diseases, trinucleotide repeat diseases, cerebral degenerative diseases, presenile dementia, senile dementia, Parkinsonism linked to chromosome 17 (FTDP-17), progressive supranuclear palsy (PSP), progressive bulbar palsy (PBP), pseudobulbar palsy, spinal and bulbar muscular atrophy (SBMA), primary lateral sclerosis, Pick's disease, primary progressive aphasia, corticobasal dementia, HIV-associated dementia, Parkinson's disease, Parkinson's disease with dementia, dementia with Lewy bodies, Down's syndrome, multiple system atrophy, spinal muscular atrophy (SMA, e.g., SMA Type I (e.g., Werdnig-Hoffnann disease) SMA Type II, SMA Type III (e.g., Kugelberg-Welander disease), or congenital SMA with arthrogryposis), progressive spinobulbar muscular atrophy (e.g., Kennedy disease), post-polio syndrome (PPS), spinocerebellar ataxia, pantothenate kinase-associated neurodegeneration (PANK), spinal degenerative disease/motor neuron degenerative diseases, upper motor neuron disorder, lower motor neuron disorder, age-related disorders and dementias, Hallervorden-Spatz syndrome, cerebral infarction, cerebral trauma, chronic traumatic encephalopathy, transient ischemic attack, Lytigo-bodig (amyotrophic lateral sclerosis-parkinsonism dementia), Guam-Parkinsonism dementia, hippocampal sclerosis, corticobasal degeneration, Alexander disease, Apler's disease, Krabbe's disease, neuroborreliosis, neurosyphilis, Sandhoff disease, Tay-Sachs disease, Schilder's disease, Batten disease, Cockayne syndrome, Kearns-Sayre syndrome, Gerstmann-Straussler-Scheinker syndrome and other transmissible spongiform encephalopathies, hereditary spastic paraparesis, Leigh's syndrome, demyelinating diseases, neuronal ceroid lipofuscinoses, epilepsy, tremors, depression, mania, anxiety and anxiety disorders, sleep disorders (e.g., narcolepsy, fatal familial insomnia), acute brain injuries (e.g., stroke, head injury), and autism, or any combination thereof.

In some embodiments, the neurodegenerative disease is amyotrophic lateral sclerosis (ALS).

In some embodiments, the neurodegenerative disease is Alzheimer's disease.

### BRIEF DESCRIPTION OF THE DRAWINGS

FIG. 1 depicts Compound 183 preventing TDP-43 nuclear clearance following proteasome inhibition. Representative micrographs of iP11NA human motor neurons unstressed, those treated with MG-132 (1 μM), or a combination of Compound 183 (500 nM) and MG-132 (1 μM) immunostained for TDP-43 (green), β-III tubulin (red) and counterstained with Hoechst (blue). Magnified images from the 3 conditions highlight the change from nuclear TDP-43 in unstressed neurons, to diffuse staining in cells under proteasomal stress, to maintenance of nuclear TDP-43 in the presence of Compound 183.

FIG. 2 depicts Compound 183 maintaining nuclear TDP-43 in a dose-dependent manner. (a) Representative micrographs of iP11NA human motor neurons treated with MG-132 (1 μM) and Compound 183 (indicated concentrations) immunostained for TDP-43 (green), β-III tubulin (pink), human nuclear antigen (red) and counterstained with Hoechst (blue). (b) Differential TDP-43 immunostaining intensity (nuclear (ROI)-cytoplasmic (ROI)) for human motor neurons unstressed (gray) or treated with MG-132 (red, 1 μM) and Compound 183 (indicated concentration). Data are displayed as dots representing replicate wells with mean and S.D. Statistical analysis performed with one-way ANOVA and Dunnett's multiple caparisons test.

FIG. 3 depicts Compound 183 reducing aggregated TDP-43, a pathological hallmark of ALS, and restoring nuclear TDP-43 localization. Immunoblot analysis of TDP-43 in the indicated fractions from neurons unstressed, treated with MG-132 (1 μM), or a combination of Compound 183 (500 nM) and MG-132 (1 μM) Data are displayed as bars with S.D. Statistical analysis performed with one-way ANOVA and Dunnett's multiple caparisons test.

FIG. 4 depicts proteasome inhibition induces splicing changes associated with TDP-43 loss-of-function. qRT-PCR analysis of TDP-43 sentinel transcripts (STMN2, ELAVL3, and PFKP) expression in human motor neurons treated with puromycin (10 mg/mL) or MG-132 (1 μM) relative unstressed samples. Data are displayed as dots representing replicate wells with mean and S.D.

FIG. 5 depicts Compound 183 restoring splicing function of TDP-43 that is lost in ALS. qRT-PCR analysis of TDP-43 sentinel transcripts (STMN2, ELAVL3, and PFKP) expression for human motor neurons treated with MG-132 (1 μM) and Compound 183 (500 nM) relative to MG-132 (1 μM) control.

FIG. 6 depicts Compound 183 restoring TDP-43 splicing function in dose dependent manner. qRT-PCR analysis of TDP-43 sentinel transcripts (STMN2, ELAVL3, and PFKP) expression for human motor neurons unstressed (gray) or treated with MG-132 (1 μM) and Compound 183 (indicated concentration) relative to MG-132 (1 μM) control. Data are displayed as dots representing replicate wells with mean and S.D. Statistical analysis performed with one-way ANOVA and Dunnett's multiple caparisons test.

### DETAILED DESCRIPTION

Amyotrophic lateral sclerosis (ALS), also known as Lou Gehrig's disease or Charcot disease, is a fatal neurodegenerative disease that occurs with an incidence of approximately 1/100,000. There is currently no therapy for ALS, and the average survival time of patients from the onset of the disease is roughly four years. ALS presents with motor weakness in the distal limbs that rapidly progresses proximally. Studies over the past decade have indicated that TDP-43 is the major protein that accumulates in affected motor neurons in sporadic ALS. The causes of sporadic ALS are not known, but identification of the major pathological species accumulating in the spinal cord of ALS patients represents a seminal advance for ALS research. To date, TDP-43 is the only protein that has been both genetically and pathologically linked with sporadic ALS, which represents the predominant form of the disease. Multiple papers have identified mutations in TDP-43 associated with sporadic and familial ALS. Inhibitors of cell death and inclusions linked to TDP-43 represent a novel therapeutic approach to ALS, and may also elucidate the biochemical pathway linked to the formation of TDP-43 inclusions. As such, TDP-43 represents one of the most promising targets for pharmacotherapy of ALS.

TDP-43 is a nuclear RNA binding protein that translocates to the cytoplasm in times of cellular stress, where it forms cytoplasmic inclusions. These inclusions then colocalize with reversible protein-mRNA aggregates termed “stress granules” (SGs). Under many stress-inducing conditions (e.g., arsenite treatment, nutrient deprivation), TDP-43 can co-localize with SGs. The reversible nature of SG-based aggregation offers a biological pathway that might be applied to reverse the pathology and toxicity associated with TDP-43 inclusion formation. Studies show that agents that inhibit SG formation also inhibit formation of TDP-43 inclusions. The relationship between TDP-43 and stress granules is important because it provides a novel approach for dispersing TDP-43 inclusions using physiological pathways that normally regulate this reversible SG process. Investigating the particular elements of the SG pathway that regulate TDP-43 inclusion formation can identify selective approaches for therapeutic intervention to delay or halt the progression of disease. Stress granule biology also regulates autophagy and apoptosis, both of which are linked to neurodegeneration. Hence, compounds inhibiting TDP-43 aggregation may play a role in inhibiting neurodegeneration.

Tau is a protein known to be present in a number of pathological conditions, particularly neurodegenerative disorders including Alzheimer's disease. Hence, compounds inhibiting tau aggregation may play a role in inhibiting neurodegeneration.

### Compounds

Accordingly, in an aspect, the disclosure provides a compound of Formula (I):

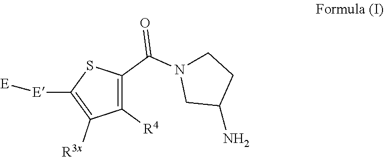

or stereoisomer and/or a pharmaceutically acceptable salt thereof, wherein:
E is C₃-C₇ cycloalkyl, heterocycloalkyl, aryl, heteroaryl, carbocyclyl, or heterocyclyl, and E may be optionally substituted;
E′ is absent, or E′ is C₃-C₇ cycloalkyl, heterocycloalkyl, aryl, heteroaryl, carbocyclyl, or heterocyclyl, and E′ may be optionally substituted; and
R^{3x} and R⁴ are each H or an independently selected optional substituent.

In some embodiments, E′ is absent. In some embodiments, E′ is C₃-C₇ cycloalkyl, heterocycloalkyl, aryl, heteroaryl, carbocyclyl, or heterocyclyl, and E′ may be optionally substituted.

In some embodiments, the compound is of Formula (Ia):

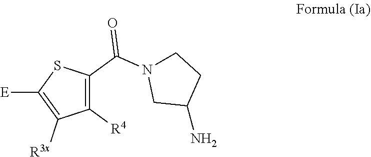

or stereoisomer and/or a pharmaceutically acceptable salt thereof.

In some embodiments, the compound is of Formula (II):

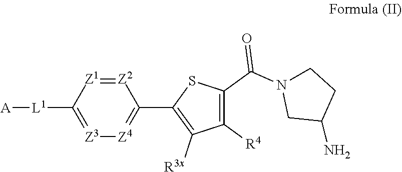

or stereoisomer and/or a pharmaceutically acceptable salt thereof, wherein:
Z¹ is N or CR¹¹;
Z² is N or CR¹²;
Z³ is N or CR¹³;
Z⁴ is N or CR¹⁴;
L¹ is absent, or L¹ is C₁-C₆ alkylene, C₁-C₆ heteroalkylene, —O—, —S—, or —NR′—, wherein the C₁-C₆ alkylene and C₁-C₆ heteroalkylene are optionally substituted;
A is H, halo, C₁-C₆ alkyl, C₁-C₆ heteroalkyl, C₁-C₆ haloalkyl, C₃-C₇ cycloalkyl, heterocycloalkyl, aryl, heteroaryl, carbocyclyl, or heterocyclyl, and A may be optionally substituted; or A and R¹¹ are taken together with the atoms to which they are attached to form an optionally substituted cycloalkyl, heterocycloalkyl, aryl, heteroaryl, carbocyclic, or heterocyclic ring;
R¹¹, R¹², R¹³, and R¹⁴ are each independently H or an optional substituent;
each R′ is H or C₁-C₆ alkyl; and
each of R^{3x} and R⁴ is independently H or an independently selected optional substituent,
wherein no more than two of Z¹, Z², Z³ and Z⁴ are N.

In some embodiments, the compound is of Formula (II):

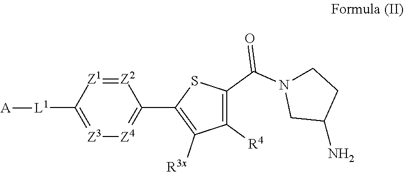

or stereoisomer and/or a pharmaceutically acceptable salt thereof, wherein:
Z¹ is N or CR¹¹;
Z² is N or CR¹²;
Z³ is N or CR¹³;
Z⁴ is N or CR¹⁴;
L¹ is absent, or L¹ is C₁-C₆ alkylene, C₁-C₆ heteroalkylene, —O—, —S—, or —NR′—, wherein the C₁-C₆ alkylene and C₁-C₆ heteroalkylene are optionally substituted by 1-4 independently substituents selected from ═O (oxo), OH, and halogen;
A is H, halo, C₁-C₁₀ alkyl, C₁-C₁₀ heteroalkyl, C₁-C₆ haloalkyl, C₃-C₇ cycloalkyl, 3-7 membered heterocycloalkyl, C₆-C₁₀ aryl, 5-6 membered heteroaryl, C₆-C₁₀ carbocyclyl, or 5-10 membered heterocyclic ring, and A is substituted by one R¹ group and optionally substituted by 1-4 independently selected R² groups; or A and R¹¹ are taken together with the atoms to which they are attached to form a C₃-C₇ cycloalkyl, aryl, 5-6 membered heteroaryl, or 3-7 membered heterocyclic ring, wherein the heteroaryl and heterocyclyl have 1-3 ring heteroatoms selected from N, O, and S, and wherein each of the rings is optionally substituted by one R¹ group and optionally substituted by 1-4 independently selected R² groups;
each of R¹¹, R¹², R¹³, and R¹⁴ is independently H or R³;
R¹ is H, C₁-C₆ alkyl, C₂-C₆ alkenyl, C₂-C₆ alkynyl, C₁-C₆ heteroalkyl, C₁-C₆ haloalkyl, —NH₂, or -L²-G;
L² is absent, or L² is C₁-C₆ alkylene, C₁-C₆ heteroalkylene, —O—, —S—, or —NR′—, wherein alkylene and heteroalkylene are optionally substituted by 1-4 substituents independently selected from ═O (oxo), OH, and halogen;
G is C₃-C₇ cycloalkyl, 3-7 membered heterocycloalkyl, aryl, 5-6 membered heteroaryl, 6-10 membered carbocyclyl, or 5-10 membered heterocyclyl, wherein the 3-7 membered heterocycloalkyl, 5-6 membered heteroaryl, and 5-10 membered heterocyclyl have 1-3 ring heteroatoms selected from N, O, and S, and wherein G may be optionally substituted by 1-4 R^{A} substituents;
each R^{A} is independently selected from C₁-C₆ alkyl, C₁-C₆ heteroalkyl, C₁-C₆ alkoxy, C₁-C₆ haloalkyl, C₁-C₆ haloalkoxy, ═O (oxo), OH, —NMe₂, —NHMe, —NH₂, CN, and halo;
each R² is independently C₁-C₆ alkyl, C₂-C₆ alkenyl, C₂-C₆ alkynyl, C₁-C₆ heteroalkyl, C₁-C₆ alkoxy, C₁-C₆ haloalkyl, C₁-C₆ haloalkoxy, ═O (oxo), —OH, —NMe₂, —NHMe, —NH₂, or halo;
each R³ is independently C₁-C₆ alkyl, C₂-C₆ alkenyl, C₂-C₆ alkynyl, C₁-C₆ alkoxy, C₁-C₆ haloalkyl, C₁-C₆ haloalkoxy, OH, cyano, or halo;
R⁴ is H, C₁-C₆ alkyl, C₂-C₆ alkenyl, C₂-C₆ alkynyl, C₁-C₆ heteroalkyl, C₁-C₆ haloalkyl, cyano, or halo;
each R′ is H or C₁-C₆ alkyl; and
R^{3x} is H, C₁-C₆ alkyl, C₂-C₆ alkenyl, C₂-C₆ alkynyl, C₁-C₆ alkoxy, C₁-C₆ haloalkyl, cyano, or halo, wherein no more than two of Z¹, Z², Z³ and Z⁴ are N.

In some embodiments, R^{3x} is H or C₁-C₆ alkyl. In some embodiments, R^{3x} is H or -Me. In some embodiments, R^{3x} is H. In some embodiments, R^{3x} is -Me.

In some embodiments, R⁴ is H, C₁-C₆ alkyl, C₂-C₆ alkenyl, or C₁-C₆ heteroalkyl.

In some embodiments, R⁴ is H, -Me, -Et, -nPr, -iPr

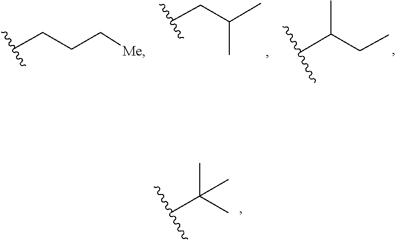

—CH₂OCH₃, —CH═CH₂, or —CH₂CH═CH₂.

In some embodiments, Z¹ is CR¹¹, Z² is CR¹², Z³ is CR¹³, and Z⁴ is CR¹⁴. In some embodiments, Z¹ is N, Z² is CR¹², Z³ is CR¹³, and Z⁴ is CR¹⁴. In some embodiments, Z¹ is CR¹¹, Z² is N, Z³ is CR¹³, and Z⁴ is CR¹⁴. In some embodiments, Z¹ is N, Z² is CR¹², Z³ is N, and Z⁴ is CR¹⁴. In some embodiments, Z¹ is N, Z² is N, Z³ is CR¹³, and Z⁴ is CR¹⁴. In some embodiments, Z¹ is CR¹¹, Z² is N, Z³ is CR¹³, and Z⁴ is N.

In some embodiments, each of R¹¹, R¹², R¹³, and R¹⁴ is independently H or R³ wherein each R³ is independently C₁-C₆ alkyl, C₁-C₆ alkoxy, C₁-C₆ haloalkyl, C₁-C₆ haloalkoxy, OH, or halo.

In some embodiments, each of R¹¹, R¹², R¹³, and R¹⁴ is independently H or R³ wherein each R³ is independently -Me, -Et, -nPr, -iPr, —CF₃, —OMe, —OCF₃, —OH, —F, or —Cl.

In some embodiments, R¹¹ is H. In some embodiments, R¹² is H. In some embodiments, R¹³ is H. In some embodiments, R¹⁴ is H. In some embodiments, each of R¹, R¹², R¹³, and R¹⁴ is H.

In some embodiments, L¹ is absent.

In some embodiments, L¹ is C₁-C₆ alkylene, C₁-C₆ heteroalkylene, or —O—.

In some embodiments, L¹ is —CH₂—, —OCH₂—, —NHCH₂—, —N(CH₃)CH₂—, or —O—. In some embodiments, L¹ is —CH₂—. In some embodiments, L¹ is —OCH₂—. In some embodiments, L¹ is —NHCH₂—. In some embodiments, L¹ is —N(CH₃)CH₂—. In some embodiments, L¹ is —O—.

In some embodiments, A is halo, C₁-C₁₀ alkyl, or C₁-C₁₀ heteroalkyl, wherein the C₁-C₁₀ alkyl and C₁-C₁₀ heteroalkyl are optionally substituted by ═O (oxo).

In some embodiments, A is —F, —Cl, -Me, -Et, -nPr, -iPr,

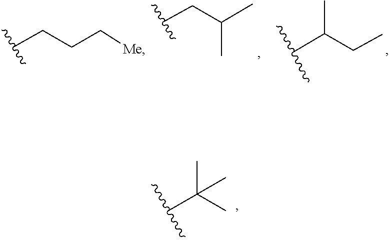

—NHCH₃, —N(CH₃)₂,

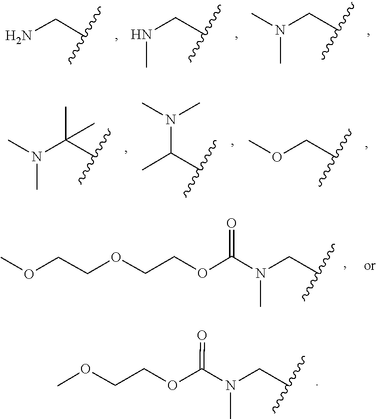

In some embodiments, A is C₃-C₇ cycloalkyl, 3-7 membered heterocycloalkyl, C₆-C₁₀ aryl, 5-6 membered heteroaryl, C₆-C₁₀ carbocyclyl, or 5-10 membered heterocyclic ring, and A is substituted by one R¹ group and optionally substituted by 1-4 independently selected R² groups.

In some embodiments, A is C₃-C₇ cycloalkyl, 3-7 membered heterocycloalkyl, C₆-C₁₀ aryl, or 5-6 membered heteroaryl, and A is substituted by one R¹ group and optionally substituted by 1-4 independently selected R² groups.

In some embodiments, A is

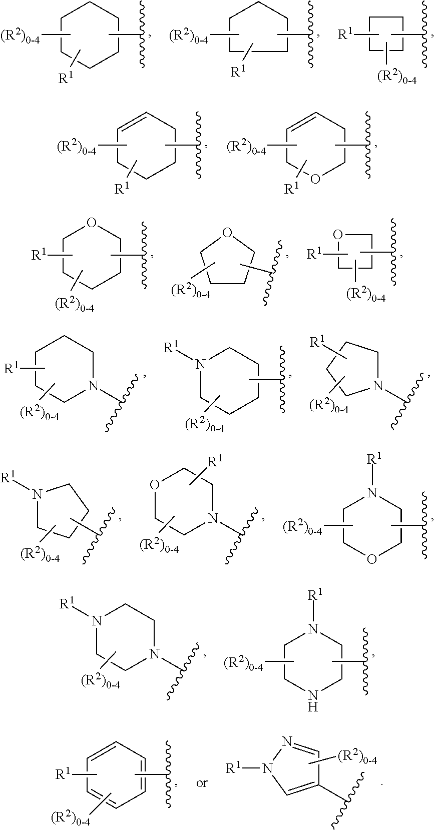

In some embodiments, A is

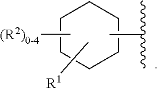

In some embodiments, A is

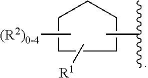

In some embodiments, A is

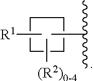

In some embodiments, A is

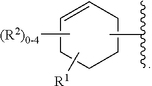

In some embodiments, A is

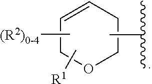

In some embodiments, A is

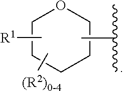

In some embodiments, A is

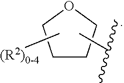

In some embodiments, A is

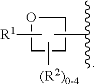

In some embodiments, A is

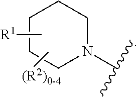

In some embodiments, A is

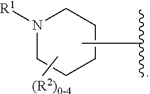

In some embodiments, A is

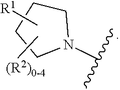

In some embodiments, A is

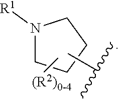

In some embodiments, A is

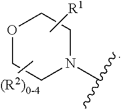

In some embodiments, A is

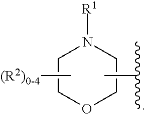

In some embodiments, A is

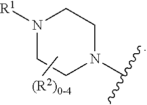

In some embodiments, A is

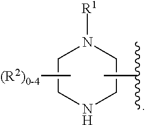

In some embodiments, A is

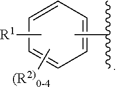

In some embodiments, A is

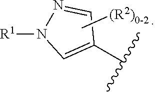

In some embodiments, A is

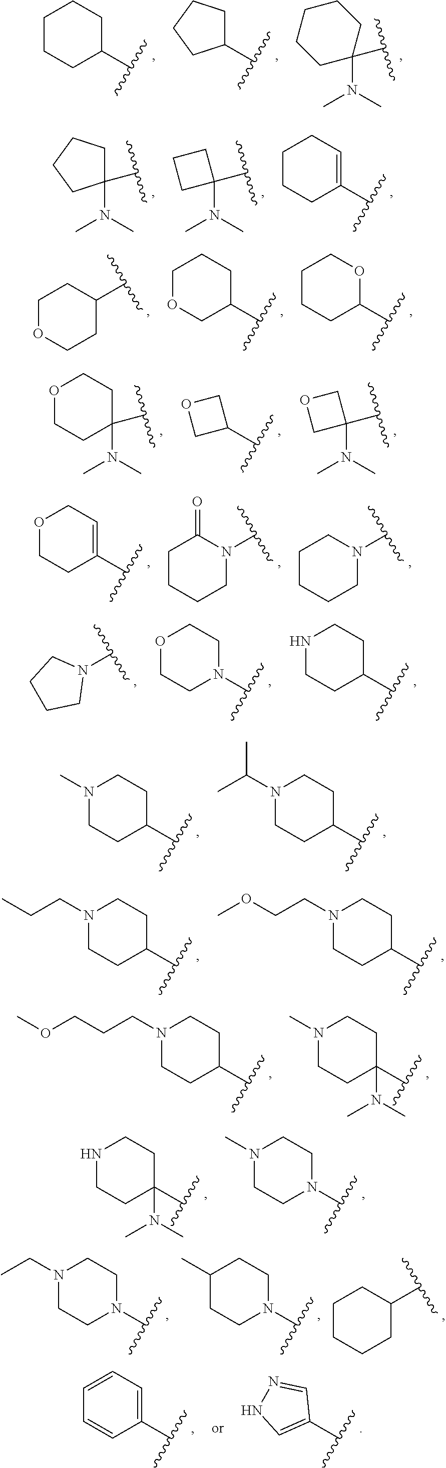

In some embodiments, A is

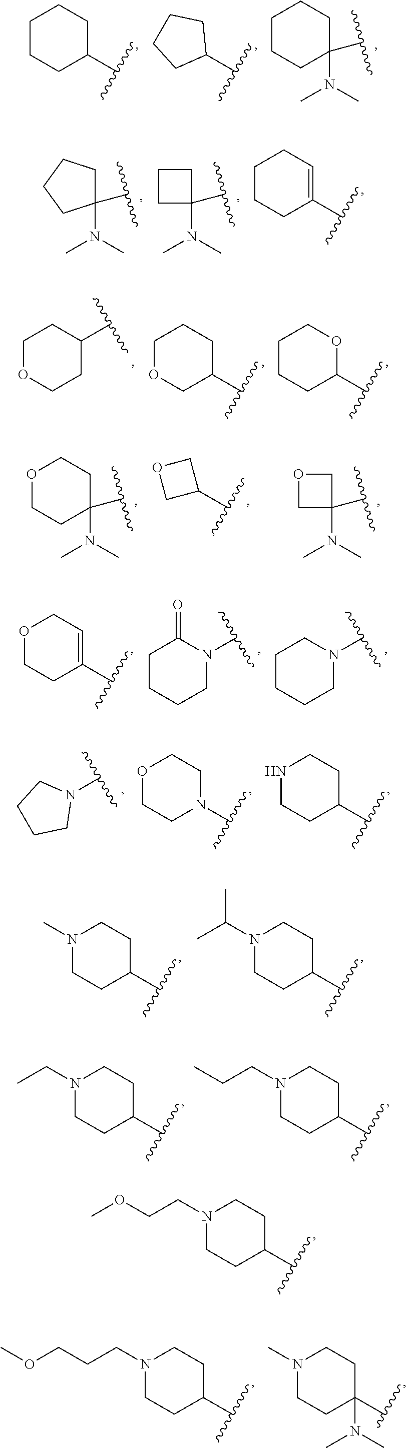

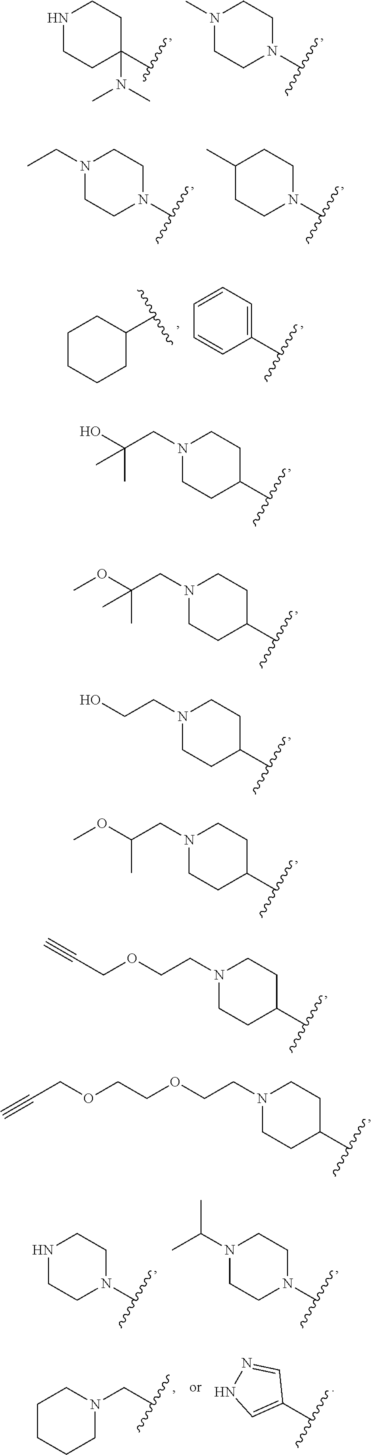

In some embodiments, each R² is independently C₁-C₆ alkyl, C₁-C₆ alkoxy, C₁-C₆ haloalkyl, C₁-C₆ haloalkoxy, ═O (oxo), —OH, —NMe₂, —NHMe, —NH₂, or halo.

In some embodiments, each R² is independently -Me, -Et, -nPr, -iPr, —CF₃, —OMe, —OCF₃, ═O (oxo), —OH, —NMe₂, —NHMe, —NH₂, F, Cl, or Br.

In some embodiments, R¹ is H or -L²-G. In some embodiments, R¹ is H. In some embodiments, R¹ is -L²-G.

In some embodiments, L² is absent. In some embodiments, L² is C₁-C₆ alkylene, C₁-C₆ heteroalkylene, or —O—. In some embodiments, L² is —CH₂—.

In some embodiments, G is C₃-C₇ cycloalkyl or 3-7 membered heterocycloalkyl, wherein the 3-7 membered heterocycloalkyl has 1-3 ring heteroatoms selected from N, O, and S, and wherein G may be optionally substituted by 1-4 R^{A} substituents.

In some embodiments, G is 3-7 membered heterocycloalkyl, wherein G may be optionally substituted by 1-4 R^{A} substituents.

In some embodiments, G is

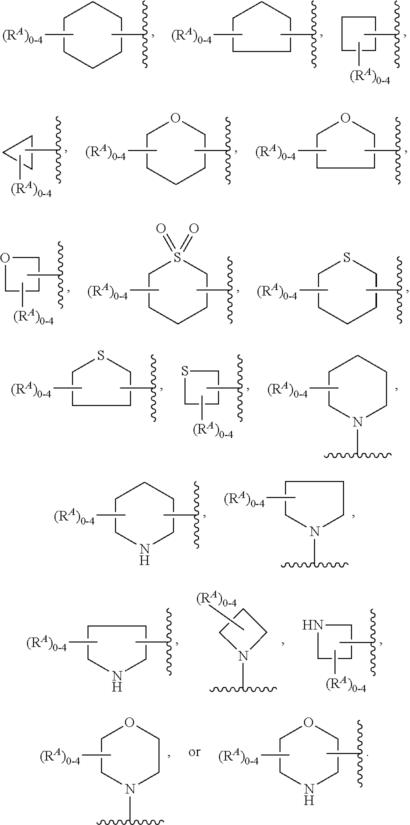

In some embodiments, each R^{A} is independently selected from C₁-C₆ alkyl.

In some embodiments, G is

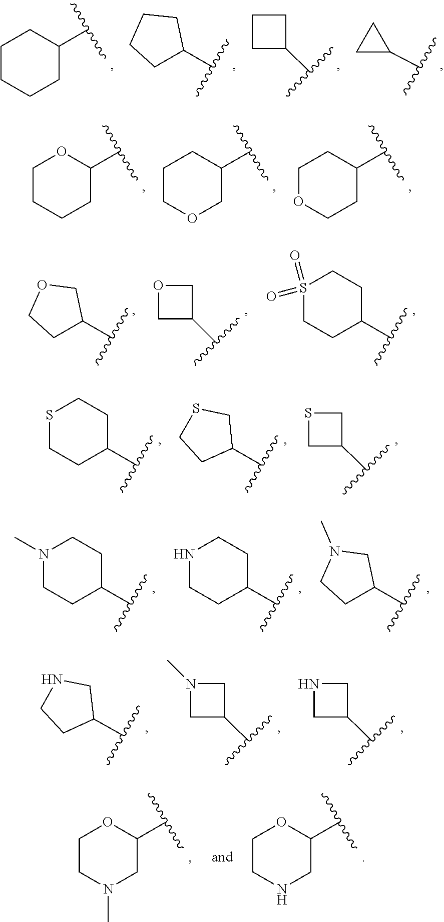

In some embodiments, A is

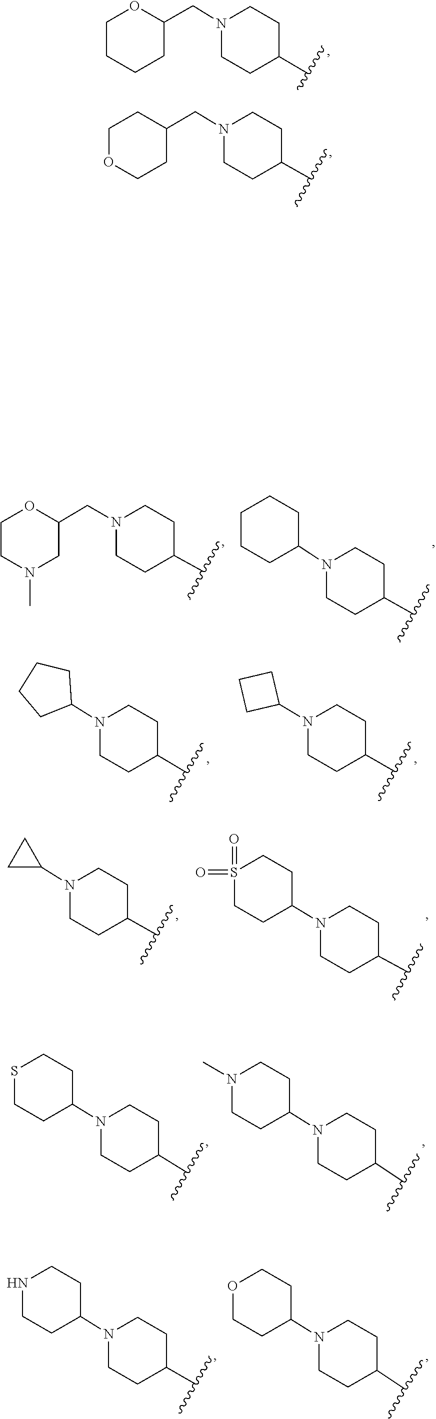

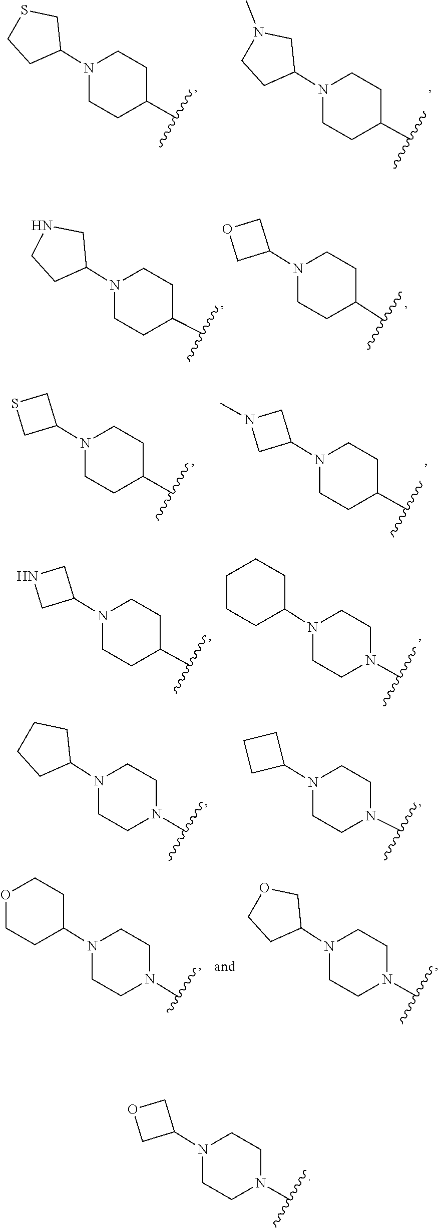

In some embodiments, A is

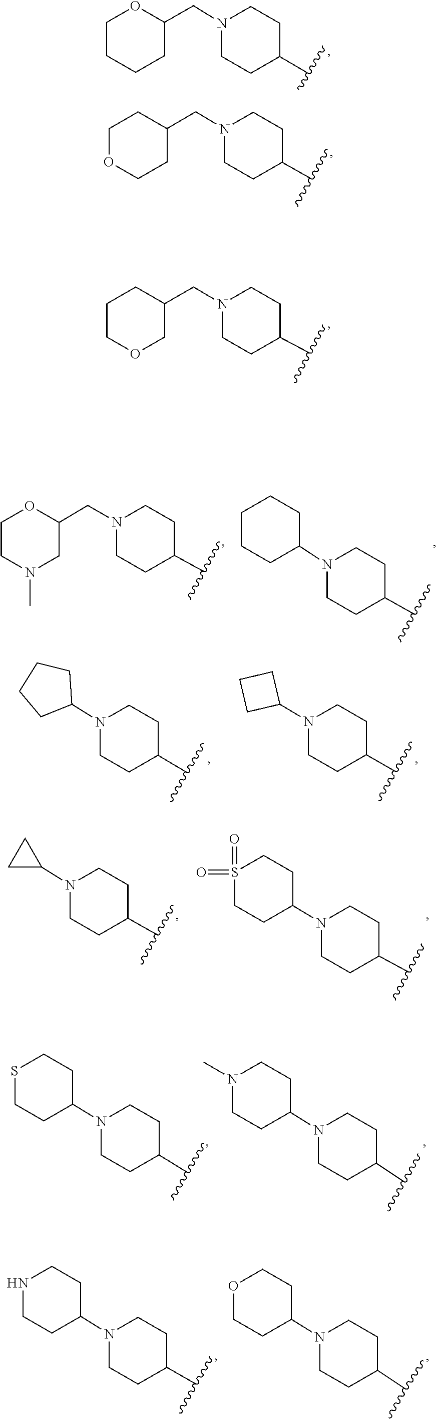

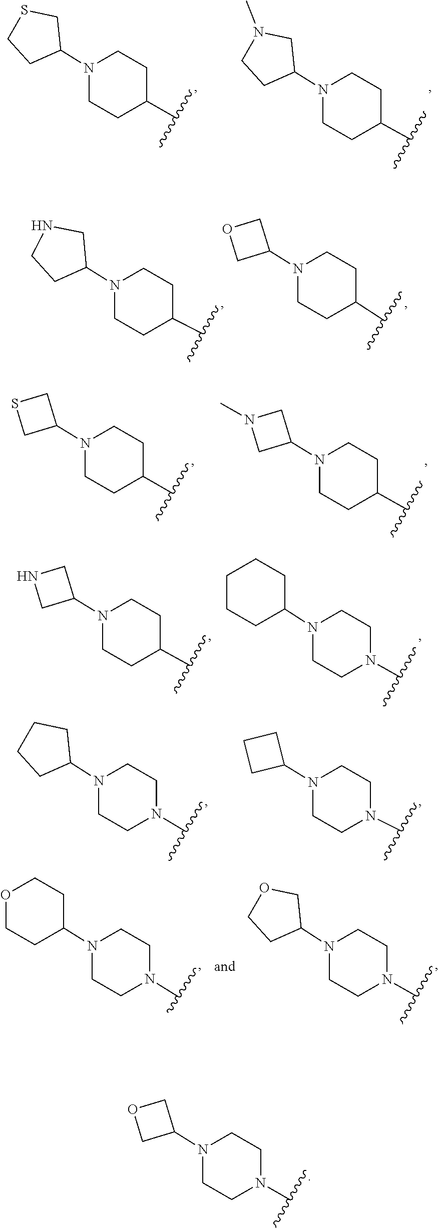

In some embodiments, A and R¹¹ are taken together with the atoms to which they are attached to form a C₃-C₇ cycloalkyl, aryl, 5-6 membered heteroaryl, or 3-7 membered heterocyclic ring, wherein the heteroaryl and heterocyclyl have 1-3 ring heteroatoms selected from N, O, and S, each of the rings optionally substituted by one R¹ group and optionally substituted by 1-4 independently selected R² groups.

In some embodiments,

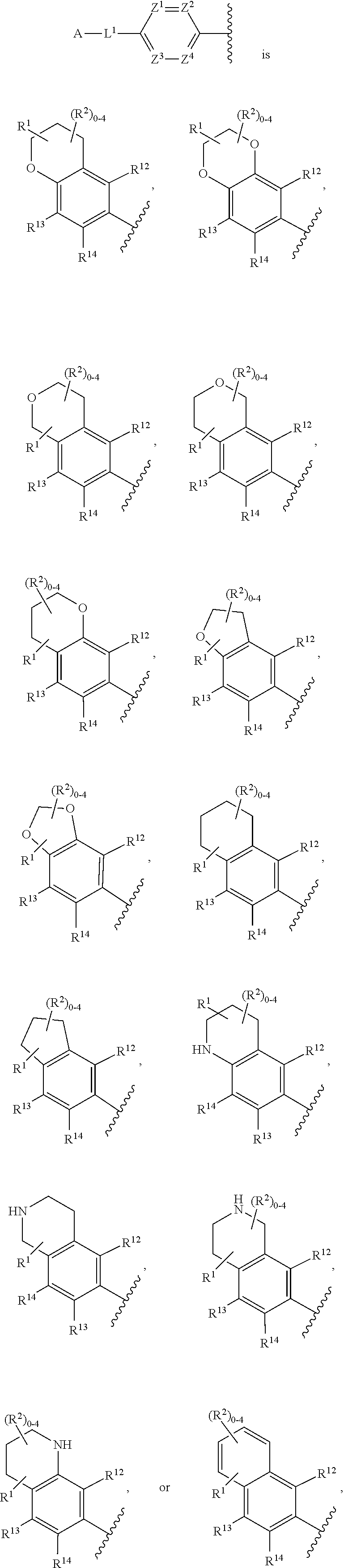

In some embodiments,

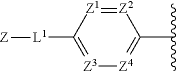

is

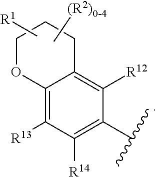

In some embodiments,

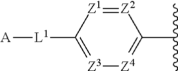

is

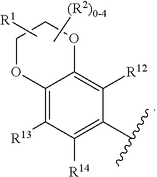

In some embodiments,

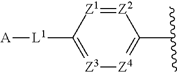

is

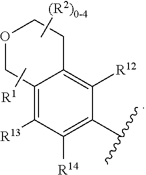

In some embodiments,

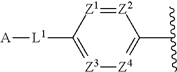

is

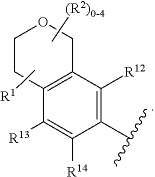

In some embodiments,

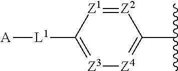

is

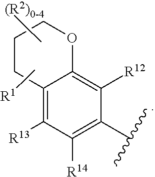

In some embodiments,

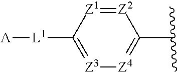

is

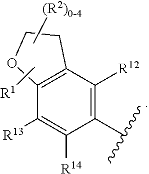

In some embodiments,

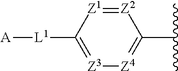

is

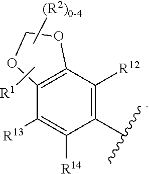

In some embodiments,

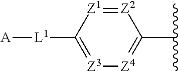

is

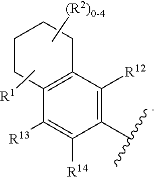

In some embodiments,

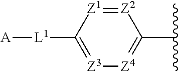

is

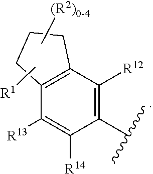

In some embodiments,

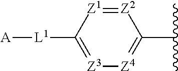

is

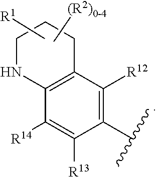

In some embodiments,

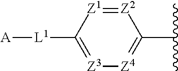

is

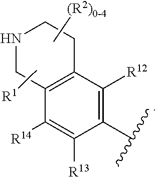

In some embodiments,

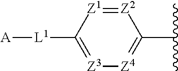

is

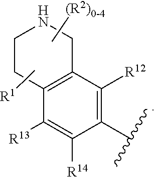

In some embodiments,

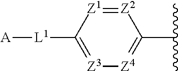

is

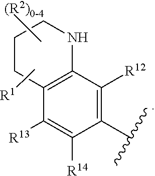

In some embodiments,

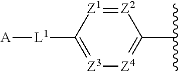

is

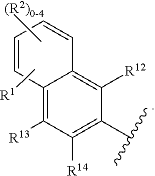

In some embodiments,

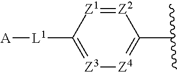

is

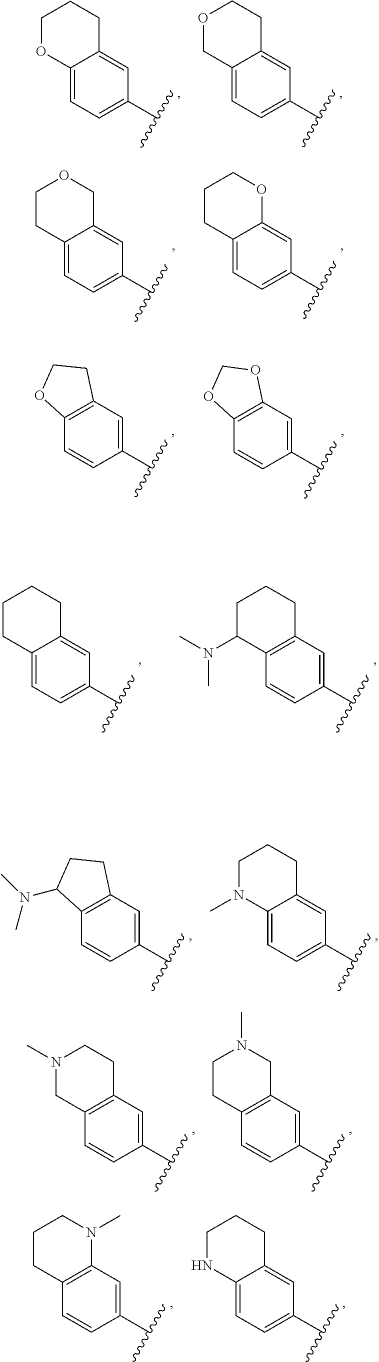

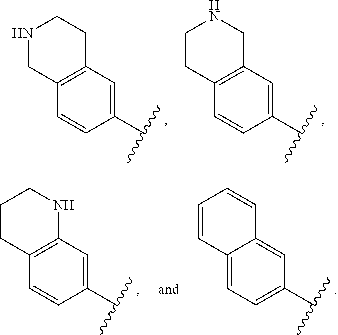 [SMILES: *c1ccc2c(c1)CCNC2 ; *c1ccc2c(c1)CNCC2 ; *c1ccc2c(c1)NCCC2 ; *c1ccc2ccccc2c1]

In some embodiments,

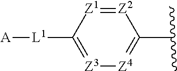

is

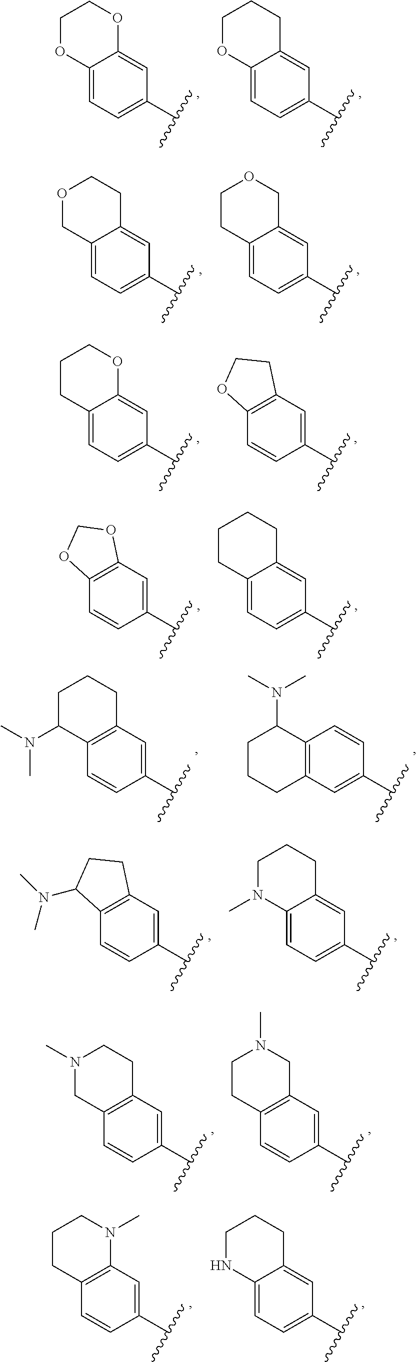

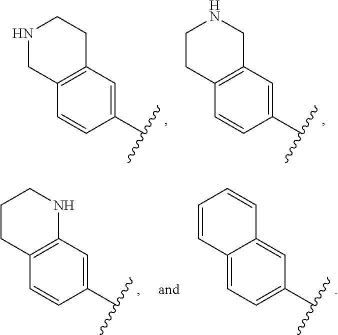 [SMILES: *c1ccc2c(c1)CCNC2 ; *c1ccc2c(c1)CNCC2 ; *c1ccc2c(c1)NCCC2 ; *c1ccc2ccccc2c1]

In some embodiments, the compound is of Formula (IIa):

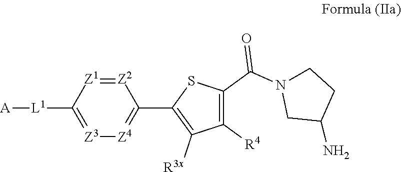

or stereoisomer and/or a pharmaceutically acceptable salt thereof.

In some embodiments, the compound is of Formula (IIb):

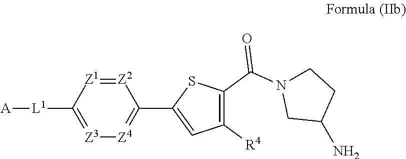

or stereoisomer and/or a pharmaceutically acceptable salt thereof.

In some embodiments, the compound is of Formula (IIc):

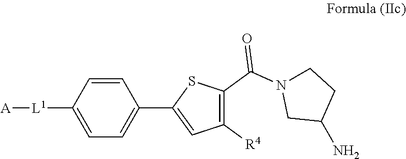

or stereoisomer and/or a pharmaceutically acceptable salt thereof.

In some embodiments, the compound is of Formula (IId):

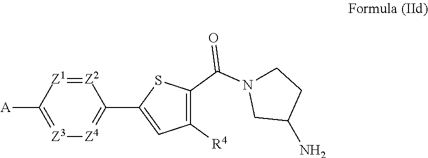

or stereoisomer and/or a pharmaceutically acceptable salt thereof.

In some embodiments, the compound is of Formula (III):

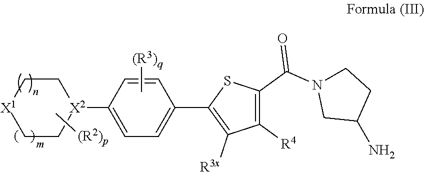

or stereoisomer and/or a pharmaceutically acceptable salt thereof, wherein:
X¹ is NR¹, O, S, SO₂, CH₂ or CHR¹;
X² is N or CH;
R¹ is H, C₁-C₆ alkyl, C₂-C₆ alkenyl, C₂-C₆ alkynyl, C₁-C₆ heteroalkyl, C₁-C₆ haloalkyl, —NH₂, or -L²-G;
L² is absent, or L² is C₁-C₆ alkylene, C₁-C₆ heteroalkylene, —O—, —S—, or —NR′—, wherein alkylene and heteroalkylene are optionally substituted by 1-4 substituents independently selected from ═O (oxo), OH, and halogen;
G is C₃-C₇ cycloalkyl, 3-7 membered heterocycloalkyl, aryl, 5-6 membered heteroaryl, 6-10 membered carbocyclyl, or 5-10 membered heterocyclyl, wherein heterocycloalkyl, heteroaryl, and heterocyclyl have 1-3 ring heteroatoms selected from N, O, and S, and wherein G may be optionally substituted by 1-4 R^{A} substituents;
each R^{A} is independently selected from C₁-C₆ alkyl, C₁-C₆ heteroalkyl, C₁-C₆ haloalkyl, C₁-C₆ haloalkoxy, OH, CN, or halo;
each R² is independently C₁-C₆ alkyl, C₂-C₆ alkenyl, C₂-C₆ alkynyl, C₁-C₆ heteroalkyl, C₁-C₆ alkoxy, C₁-C₆ haloalkyl, C₁-C₆ haloalkoxy, —NH₂, or halo;
each R³ is independently C₁-C₆ alkyl, C₂-C₆ alkenyl, C₂-C₆ alkynyl, C₁-C₆ alkoxy, C₁-C₆ haloalkyl, C₁-C₆ haloalkoxy, OH, cyano, or halo;
R^{3x} is H, C₁-C₆ alkyl, C₂-C₆ alkenyl, C₂-C₆ alkynyl, C₁-C₆ alkoxy, C₁-C₆ haloalkyl, cyano, or halo;
R⁴ is H, C₁-C₆ alkyl, C₂-C₆ alkenyl, C₂-C₆ alkynyl, C₁-C₆ heteroalkyl, C₁-C₆ haloalkyl, cyano, or halo;
each R′ is H or C₁-C₆ alkyl;
m is 0 or 1;
n is 0 or 1;
p is 0, 1, 2, 3, or 4; and
q is 0, 1, 2, 3, or 4.

In some embodiments, the compound is of Formula (III):

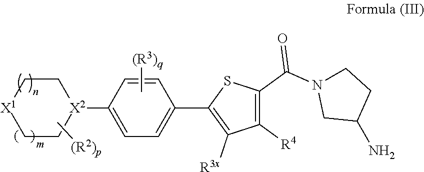

or stereoisomer and/or a pharmaceutically acceptable salt thereof, wherein:
X¹ is NR¹, O, S, SO₂, CH₂ or CHR¹;
X² is N or CH;
R¹ is H, C₁-C₆ alkyl, C₂-C₆ alkenyl, C₂-C₆ alkynyl, C₁-C₆ heteroalkyl, C₁-C₆ haloalkyl, —NH₂, or -G;
G is C₃-C₇ cycloalkyl, 3-7 membered heterocycloalkyl, aryl, 5-6 membered heteroaryl, 6-10 membered carbocyclyl, or 5-10 membered heterocyclyl, wherein heterocycloalkyl, heteroaryl, and heterocyclyl have 1-3 ring heteroatoms selected from N, O, and S, and wherein G may be optionally substituted by 1-4 R^{A} substituents;
each R^{A} is independently selected from C₁-C₆ alkyl, C₁-C₆ heteroalkyl, C₁-C₆ haloalkyl, C₁-C₆ haloalkoxy, OH, CN, or halo;
each R² is independently C₁-C₆ alkyl, C₁-C₆ haloalkyl, or halo;
each R³ is independently C₁-C₆ alkyl, C₁-C₆ alkoxy, C₁-C₆ haloalkyl, cyano, or halo;
R^{3x} is H, C₁-C₆ alkyl, C₂-C₆ alkenyl, C₂-C₆ alkynyl, C₁-C₆ alkoxy, C₁-C₆ haloalkyl, cyano, or halo;
R⁴ is H, C₁-C₆ alkyl, C₁-C₆ alkoxy, C₁-C₆ haloalkyl, cyano, or halo;
m is 0 or 1;
n is 0 or 1;
p is 0, 1, 2, 3, or 4; and
q is 0, 1, 2, 3, or 4.

In some embodiments, X¹ is NR¹. In some embodiments, X¹ is CH₂ or CHR.

In some embodiments, X² is CH. In some embodiments, X² is N.

In some embodiments, X¹ is NR¹ and X² is CH.

In some embodiments, X¹ is CH₂ or CHR¹ and X² is N.

In some embodiments, X¹ is NR¹ and X² is N.

In some embodiments, R¹ is C₁-C₆ alkyl.

In some embodiments, R¹ is -Me, -Et, -nPr, -iPr,

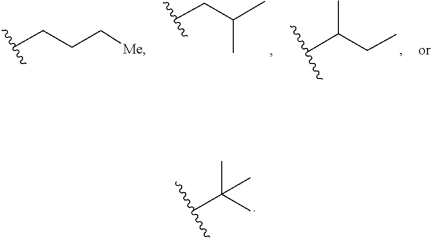

In some embodiments, R^{3x} is H or C₁-C₆ alkyl. In some embodiments, R^{3x} is H or -Me. In some embodiments, R^{3x} is H. In some embodiments, R^{3x} is -Me.

In some embodiments, R⁴ is C₁-C₆ alkyl. In some embodiments, R⁴ is -Me, -Et, -nPr, -iPr,

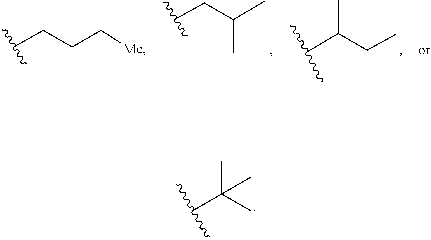

In some embodiments, R⁴ is -Me. In some embodiments, R⁴ is halo, C₁-C₆ haloalkoxy, or C₁-C₆ alkoxy. In some embodiments, R⁴ is —Cl, —Br, —OCF₃, —OMe, or

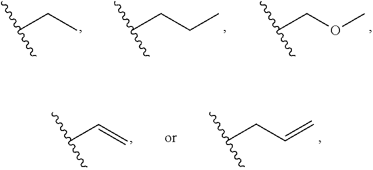

In some embodiments, m is 0. In some embodiments, m is 1.

In some embodiments, n is 0. In some embodiments, n is 1.

In some embodiments, m is 1 and n is 1. In some embodiments, m is 0 and n is 1.

In some embodiments, p is 0. In some embodiments, p is 1. In some embodiments, p is 2. In some embodiments, p is 3. In some embodiments, p is 4.

In some embodiments, q is 0. In some embodiments, q is 1. In some embodiments, q is 2. In some embodiments, q is 3. In some embodiments, q is 4.

In some embodiments, the compound is of Formula (IIIa):

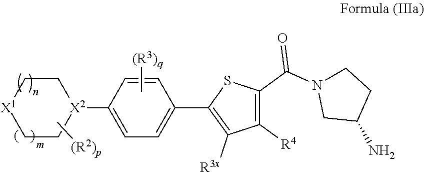

or stereoisomer and/or a pharmaceutically acceptable salt thereof.

In some embodiments, the compound is of Formula (IIIb):

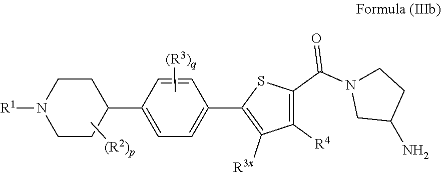

or stereoisomer and/or a pharmaceutically acceptable salt thereof.

In some embodiments, the compound is of Formula (IIIc):

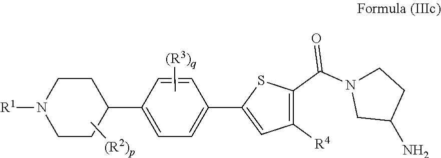

or stereoisomer and/or a pharmaceutically acceptable salt thereof.

In some embodiments, the compound is of Formula (IIId):

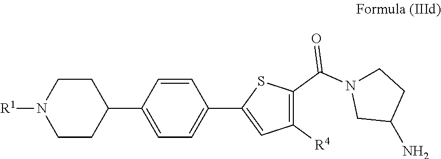

or stereoisomer and/or a pharmaceutically acceptable salt thereof.

In some embodiments, the compound is of Formula (IIIe):

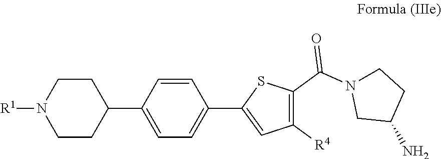

or stereoisomer and/or a pharmaceutically acceptable salt thereof.

In some embodiments, the disclosure provides a compound of Formula (IV):

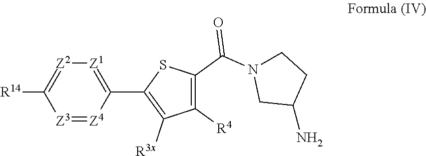

or stereoisomer and/or a pharmaceutically acceptable salt thereof, wherein:
Z¹ is N or CR¹¹;
Z² is N or CR¹²;
Z³ is N or CR¹³;
Z⁴ is N or CH;
R¹¹ is selected from the group consisting of H, —OH, halo, C₁-C₆ alkyl, C₁-C₆ heteroalkyl, and C₁-C₆ haloalkoxy;
R¹² is selected from the group consisting of H, —OH, halo, C₁-C₆ alkyl, C₁-C₆ heteroalkyl, and C₁-C₆ haloalkoxy;
R¹³ is H or halo;
R¹⁴ is selected from the group consisting of H, halo, C₁-C₆ alkylene, C₁-C₆ heteroalkylene, 5-7 membered aryl, 5-7 membered heteroaryl, and 3-7 membered heterocycloalkyl, wherein the R¹⁴ may be optionally substituted with one or more R^{14a};
R^{14a} is selected from the group consisting of C₁-C₆ alkyl, C₁-C₆ heteroalkyl, C₃-C₇ cycloalkyl, 5-7 membered aryl, 3-7 membered heterocycloalkyl, and —C(O)OR^{14b}, wherein the C₁-C₆ alkyl or the 3-7 membered heterocycloalkyl may be optionally substituted with one or more R^{14b};
R^{14b} is selected from the group consisting of —OH, oxo, C₁-C₆ alkyl, C₁-C₈ heteroalkyl, C₂-C₆ alkynyl, and 3-7 membered heterocycloalkyl, wherein the 3-7 membered heterocycloalkyl may be optionally substituted with one or more C₁-C₆ alkyl or C₁-C₆ heteroalkyl;
wherein when Z² is CR¹², R¹² and R¹⁴ may be taken together with the atoms to which they are attached to form a cycloalkyl, heterocycloalkyl, or aryl, wherein the cycloalkyl or heterocycloalkyl may be optionally substituted with one or more C₁-C₆ alkyl or C₁-C₆ heteroalkyl;
R^{3x} is H or C₁-C₆ alkyl; and
R⁴ is selected from the group consisting of H, halo, C₁-C₆ alkyl, C₁-C₆ heteroalkyl, C₂-C₆ alkenyl, and C₁-C₆ haloalkoxy.

In some embodiments, Z¹ is CR¹¹, Z² is CR¹², Z³ is CR¹³, and Z⁴ is CH.

In some embodiments, when Z¹ is N, Z² is CR¹², Z³ is CR¹³, and Z⁴ is CH.

In some embodiments, when Z² is N, Z¹ is CR¹¹, Z³ is CR¹³, and Z⁴ is CH.

In some embodiments, when Z² and Z³ are N, Z¹ is CR¹¹ and Z⁴ is CH.

In some embodiments, when Z² and Z⁴ are N, Z¹ is CR¹¹ and Z³ is CR¹³.

In some embodiments, R¹¹ is selected from the group consisting of halo, C₁-C₆ alkyl, C₁-C₆ heteroalkyl, and C₁-C₆ haloalkoxy.

In some embodiments, R¹² is selected from the group consisting of halo, C₁-C₆ alkyl, C₁-C₆ heteroalkyl, and C₁-C₆ haloalkoxy.

In some embodiments, R¹¹ is selected from the group consisting of -Me, -Et, —Cl, —F, —OMe, and —OCF₃.

In some embodiments, R¹² is selected from the group consisting of -Me, -Et, —Cl, —F, —OMe, and —OCF₃.

In some embodiments, R¹¹ is —OH.

In some embodiments, R¹² is —OH.

In some embodiments, R¹³ is halo.

In some embodiments, R¹³ is —Cl.

In some embodiments, R¹⁴ is selected from the group consisting of H, halo, C₁-C₃ alkylene, C₁-C₆ heteroalkylene, 6 membered aryl, 5 membered heteroaryl, and 4-6 membered heterocycloalkyl, wherein the C₁-C₃ alkylene, C₁-C₆ heteroalkylene, 6 membered aryl, 5 membered heteroaryl, and 4-6 membered heterocycloalkyl may be optionally substituted with one or more R^{14a}.

In some embodiments, R¹⁴ is selected from the group consisting of -Et, —O—, —CH₂—, -nPr, -iPr, —Cl, NHCH₃, —N(CH₃)₂,

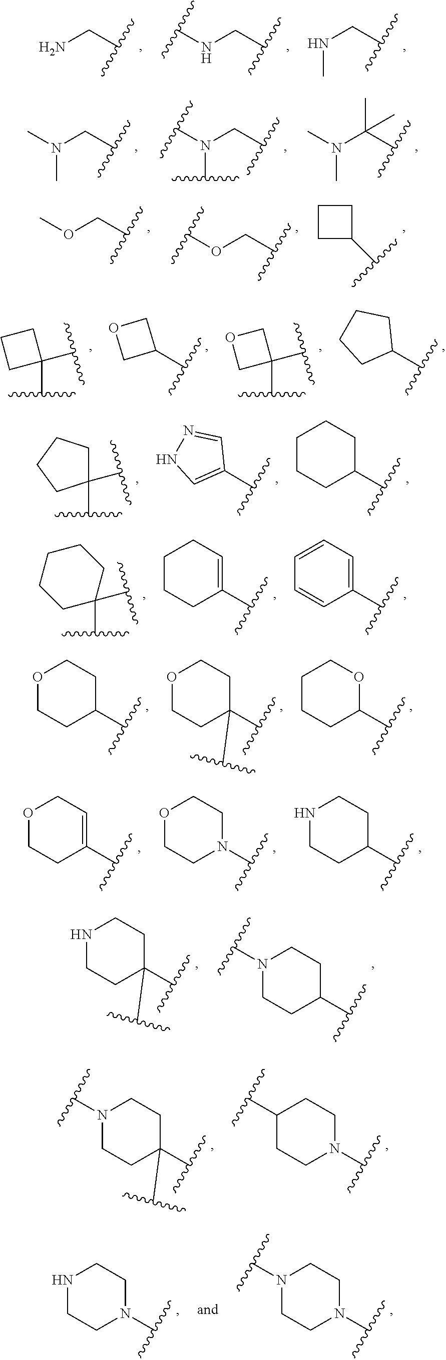

and herein if R¹⁴ contains a substitutable atom, that atom may be optionally substituted with one or more R^{14a}. In some embodiments, R¹⁴ is selected from the group consisting of -Et, —O—, —CH₂—, -nPr, -iPr, and —Cl, wherein if R¹⁴ contains a substitutable atom, that atom may be optionally substituted with one or more R^{14a}. In some embodiments, R¹⁴ is selected from the group consisting of NHCH₃, —N(CH₃)₂,

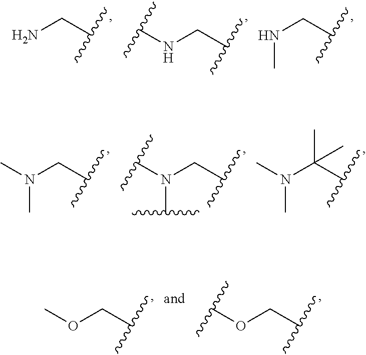

wherein if R¹⁴ contains a substitutable atom, that atom may be optionally substituted with one or more R^{14a}. In some embodiments, R¹⁴ is selected from the group consisting of

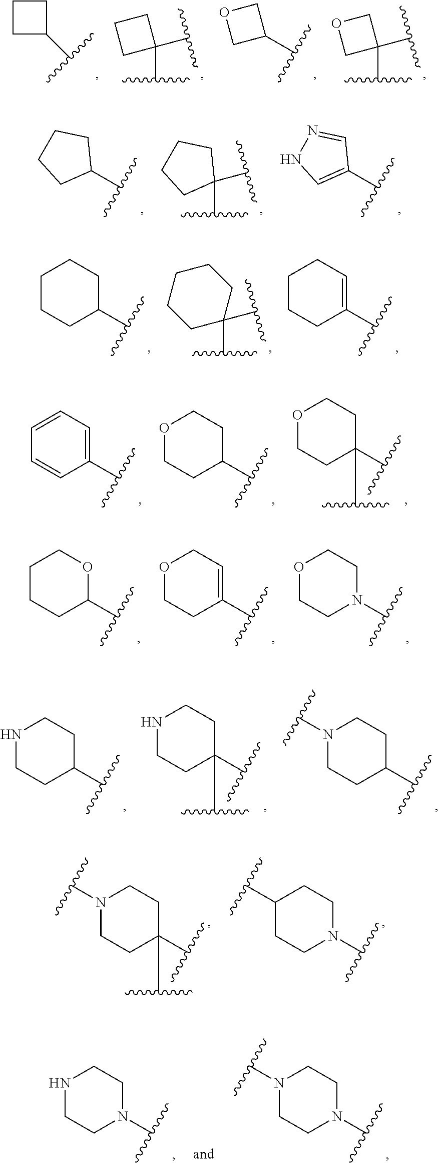

wherein if R¹⁴ contains a substitutable atom, that atom may be optionally substituted with one or more R^{14a}.

In some embodiments, R¹⁴ is selected from the group consisting of -Et, -nPr, -iPr, —Cl, NHCH₃, —N(CH₃)₂,

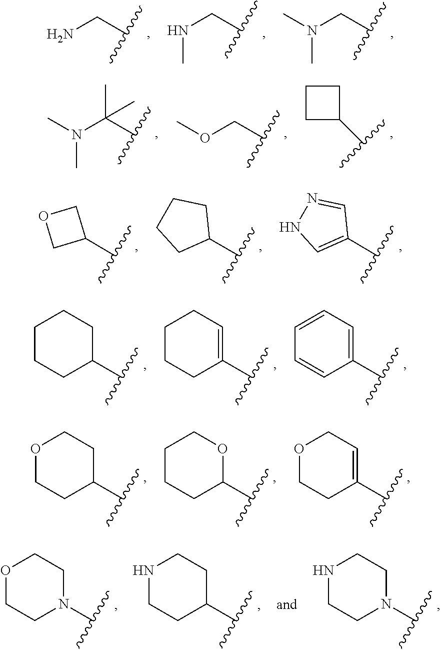

N wherein if R¹⁴ contains a substitutable atom, that atom may be optionally substituted with one or more R^{14a}. In some embodiments, R¹⁴ is selected from the group consisting of -Et, -nPr, -iPr, and —Cl, wherein if R¹⁴ contains a substitutable atom, that atom may be optionally substituted with one or more R^{14a}. In some embodiments, R¹⁴ is selected from the group consisting of NHCH₃, —N(CH₃)₂,

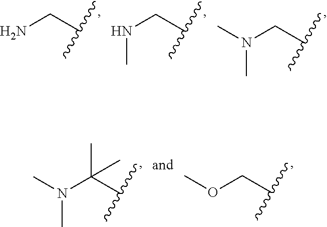

wherein if R¹⁴ contains a substitutable atom that atom may be optionally substituted with one or more R^{14a}. In some embodiments, R¹⁴ is selected from the group consisting of

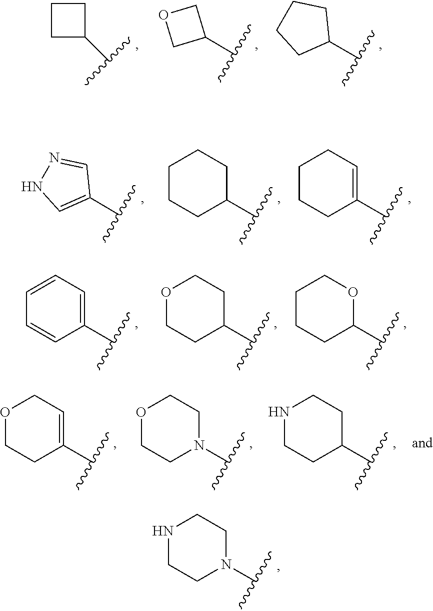

wherein if R¹⁴ contains a substitutable atom, that atom may be optionally substituted with one or more R^{14a}.

In some embodiments, R¹⁴ is selected from the group consisting of

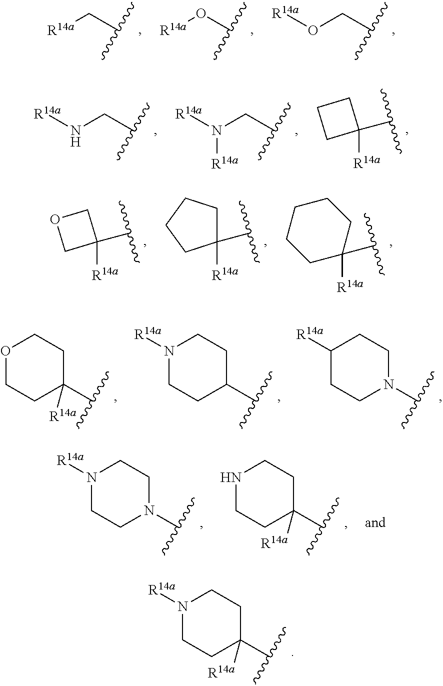

In some embodiments, R¹⁴ is selected from the group consisting of

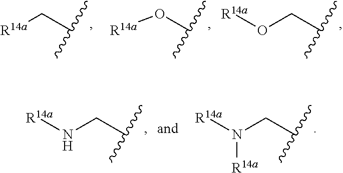

In some embodiments, R¹⁴ is selected from the group consisting of

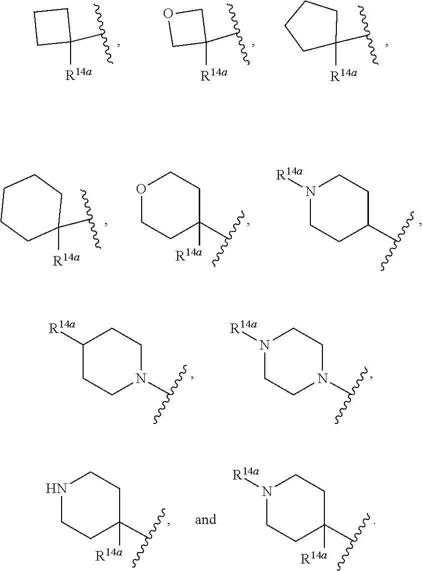

In some embodiments, R^{14a} is selected from the group consisting of C₁-C₃ alkyl, C₁-C₆ heteroalkyl, C₃-C₆ cycloalkyl, 6 membered aryl, and 4-6 membered heterocycloalkyl, wherein C₁-C₆ heteroalkyl, C₃-C₇ cycloalkyl, and 5-7 membered aryl may be optionally substituted with R^{14b}.

In some embodiments, R^{14a} is selected from the group consisting of -Me, -Et, —CH₂—, nPr,

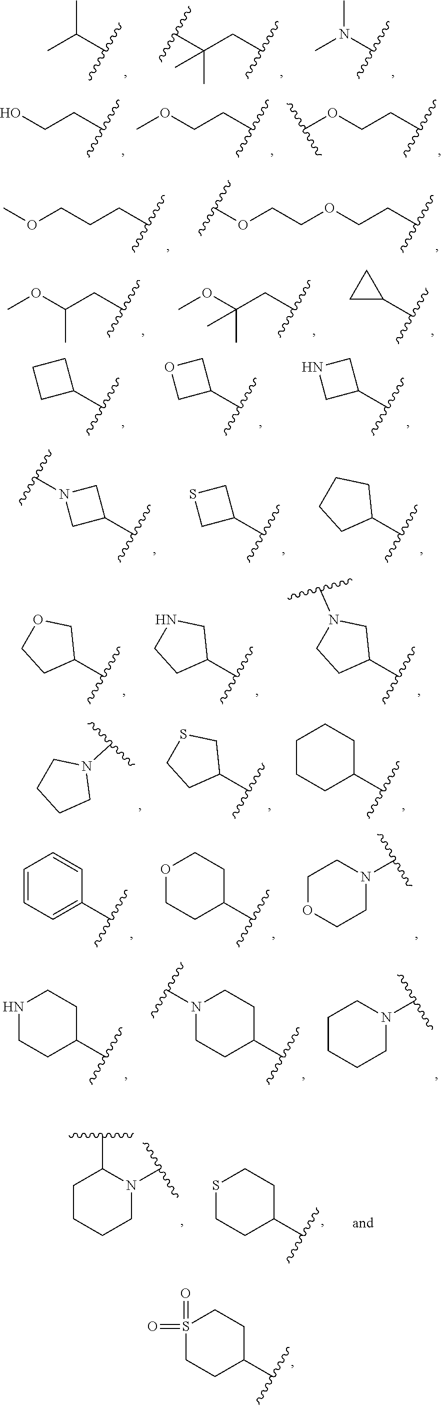

wherein if R^{14a} contains a substitutable atom, that atom may be optionally substituted with one or more R^{14b}. In some embodiments, R^{14a} is selected from the group consisting of -Me, -Et, —CH₂—, and nPr, wherein if R^{14a} contains a substitutable atom, that atom may be optionally substituted with one or more R^{14b}. In some embodiments, R^{14a} is selected from the group consisting of

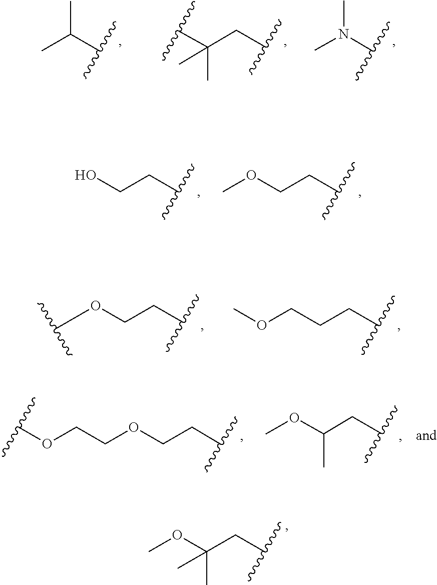

wherein if R^{14a} contains a substitutable atom, that atom may be optionally substituted with one or more R^{14b}. In some embodiments, R^{14a} is selected from the group consisting of

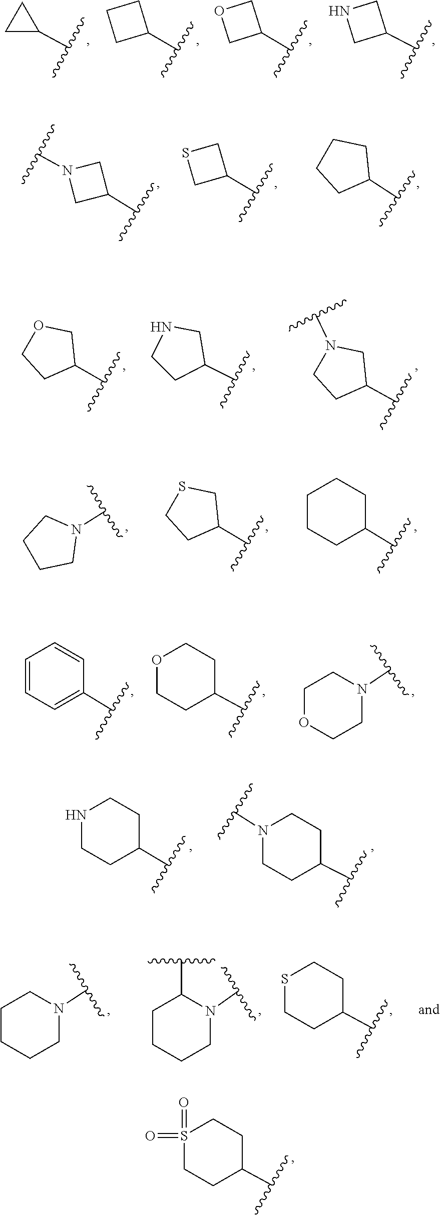

wherein if R^{14a} contains a substitutable atom, that atom may be optionally substituted with one or more R^{14b}.

In some embodiments, R^{14a} is selected from the group consisting of -Me, -Et, nPr,

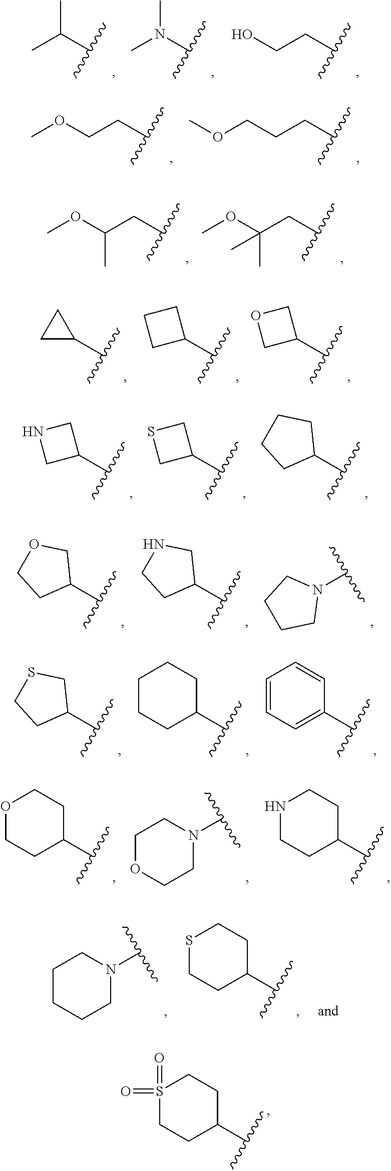

wherein if R^{14a} contains a substitutable atom, that atom may be optionally substituted with one or more R^{14b}. In some embodiments, R^{14a} is selected from the group consisting of -Me, -Et, and nPr, wherein if R^{14a} contains a substitutable atom, that atom may be optionally substituted with one or more R^{14b}. In some embodiments, R^{14a} is selected from the group consisting of

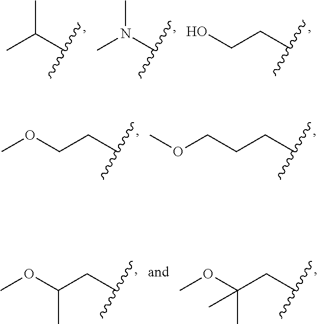

wherein if R^{14a} contains a substitutable atom, that atom may be optionally substituted with one or more R^{14b}. In some embodiments, R^{14a} is selected from the group consisting of

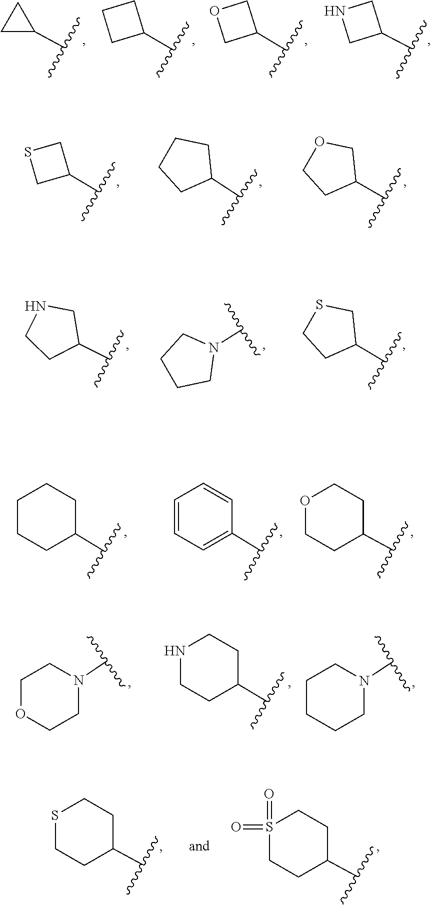

wherein if R^{14a} contains a substitutable atom, that atom may be optionally substituted with one or more R^{14b}.

In some embodiments, R^{14a} is selected from the group consisting of —C(O)OR^{14b},

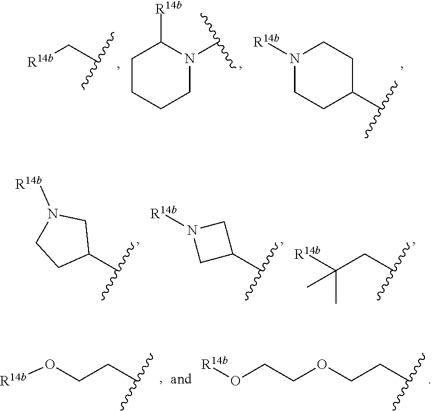

In some embodiments, R^{14b} is selected from the group consisting of oxo, —OH, -Me, C₃-C₇ heteroalkyl, C₂-C₃ alkynyl, and 6 membered heterocycloalkyl, wherein the 3-7 membered heterocycloalkyl may be optionally substituted with one or more C₁-C₆ alkyl. In some embodiments, R^{14b} is selected from the group consisting of oxo, —OH, and -Me.

In some embodiments, R^{14b} is selected from the group consisting of oxo, —OH, -Me,

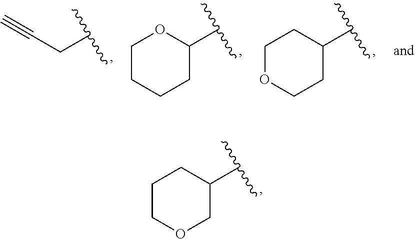

wherein the

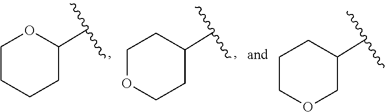

may be optionally substituted with oxo or C₁-C₆ alkyl. In some embodiments, R^{14b} is selected from the group consisting of oxo, —OH, -Me, and

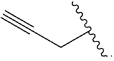 [SMILES: *CC#C]

In some embodiments, R^{14b} may be optionally substituted with oxo or C₁-C₆ alkyl.

In some embodiments, R^{14b} is selected from the group consisting of oxo, —OH, -Me,

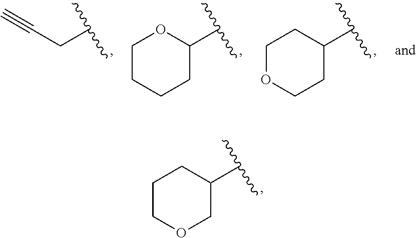

wherein the

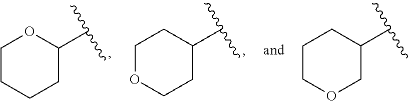 [SMILES: *C1CCCCO1 ; *C1CCOCC1 ; *C1CCCOC1]

may be optionally substituted with -Me. In some embodiments, R^{14b} may be optionally substituted with -Me.

In some embodiments, when Z² is CR¹², R¹² and R¹⁴ may be taken together with the atoms to which they are attached to form an aryl, cycloalkyl, or heterocycloalkyl, wherein the cycloalkyl or heterocycloalkyl may be optionally substituted with one or more C₁-C₆ alkyl or C₁-C₆ heteroalkyl.

In some embodiments, when Z² is CR¹², R¹² and R¹⁴ taken together are selected from the group consisting of

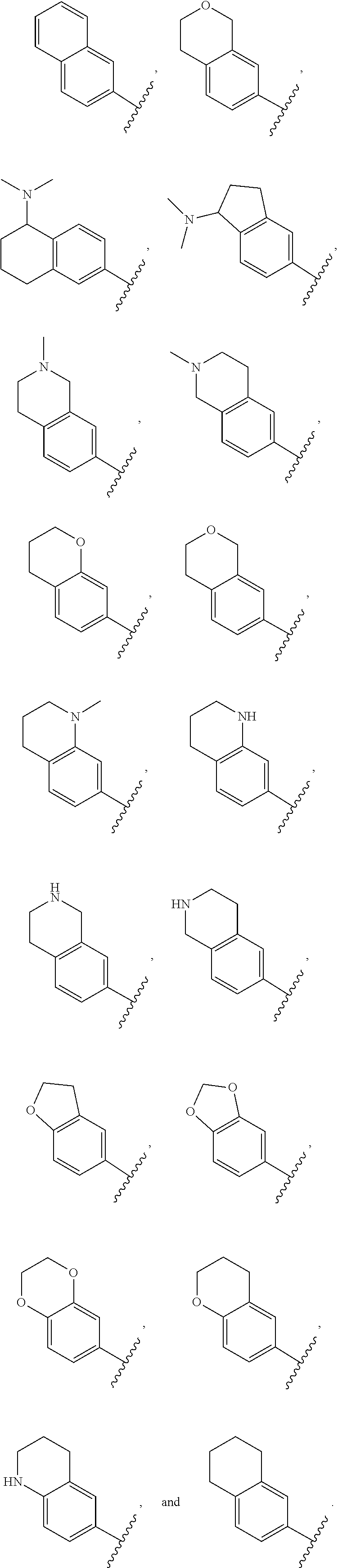

In some embodiments, R¹² and R¹⁴ taken together is

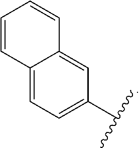 [SMILES: *c1ccc2ccccc2c1]

In some embodiments, when Z² is CR¹², R¹² and R¹⁴ taken together are selected from the group consisting of

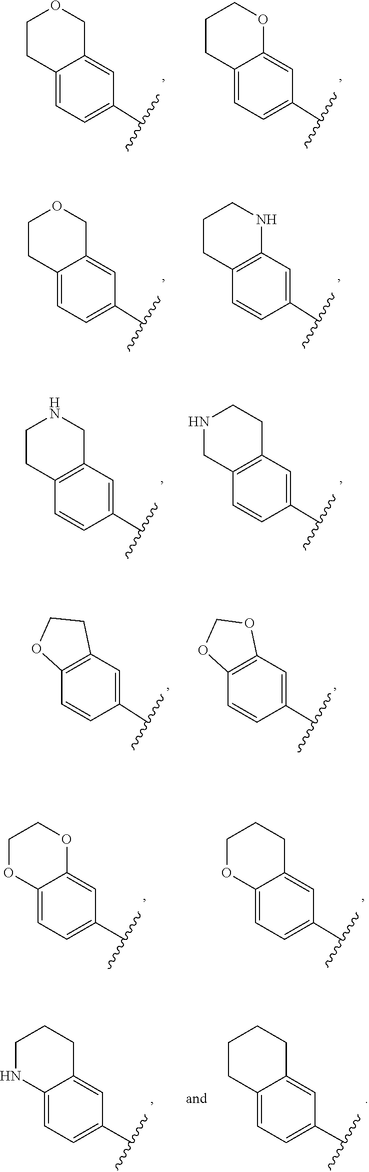

In some embodiments, when Z² is CR¹², R¹² and R¹⁴ taken together are selected from the group consisting of

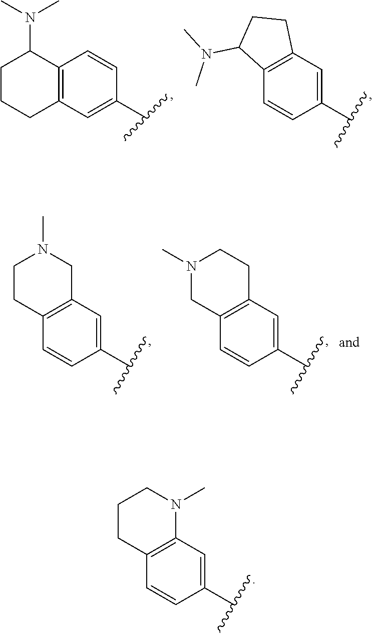 [SMILES: *c1ccc2c(c1)CCCC2N(C)C ; *c1ccc2c(c1)CCC2N(C)C ; *c1ccc2c(c1)CN(C)CC2 ; *c1ccc2c(c1)CCN(C)C2 ; *c1ccc2c(c1)N(C)CCC2]

In some embodiments, R^{3x} is C₁-C₆ alkyl. In some embodiments, R^{3x} is C₁-C₃ alkyl.

In some embodiments, R^{3x} is -Me. In some embodiments, R^{3x} is -Et. In some embodiments, R^{3x} is nPr. In some embodiments, R^{3x} is iPr.

In some embodiments, R⁴ is selected from the group consisting of halo, C₁-C₆ alkyl, C₁-C₆ heteroalkyl, C₂-C₆ alkenyl, and C₁-C₆ haloalkoxy.

In some embodiments, R⁴ is selected from the group consisting of —Cl, —Br, —OCF₃, -Me, -Et, —OMe,

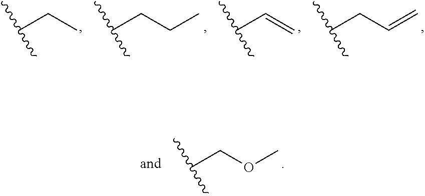

In some embodiments, R⁴ is selected from the group consisting of —Cl, —Br, —OCF₃, -Me, -Et, —OMe. In some embodiments, R4 is selected from the group consisting of

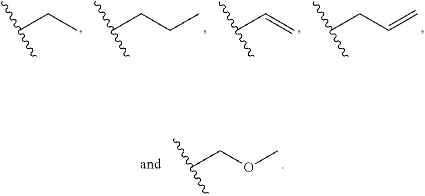

In some embodiments, the compound is selected from a compound disclosed in the specification or figures.

In some embodiments, provided is a pharmaceutical composition comprising a pharmaceutically acceptable carrier and a compound disclosed herein (e.g., a compound of Formula (I), (II), (III), or (IV), or subformulas thereof, or a compound of Table 1, or a pharmaceutically acceptable salt thereof).

In some embodiments, the compound of Formula (I), (II), (III), or (IV) is selected from a compound disclosed in the specification or figures.

In another aspect, the disclosure provides a pharmaceutical composition comprising a pharmaceutically acceptable carrier and any one of the compounds disclosed herein (e.g., a compound of Formula (I), (II), (III), or (IV), or subformulas thereof, or a compound of Table 1, or a pharmaceutically acceptable salt thereof).

In some embodiments, the compound of Formula (I), (II), (III), or (IV), or subformulas thereof, is selected from the compounds in Table 1.

TABLE 1
Exemplary compounds of the disclosure

| Compound No. | Structure |
|---|---|
| 101 | 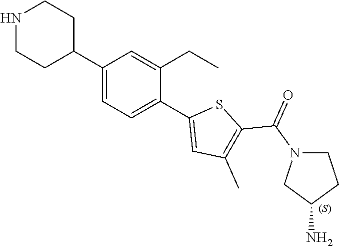 |
| 102 | 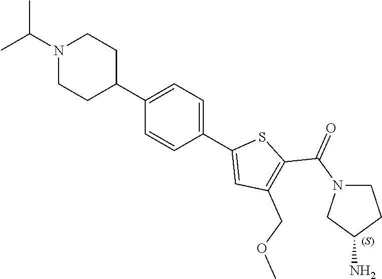 |
| 103 | 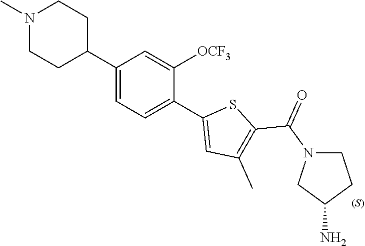 |
| 104 | 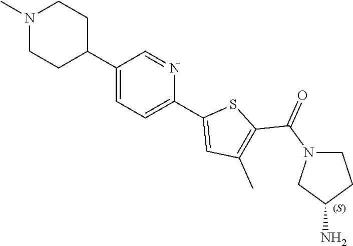 |
| 105 | 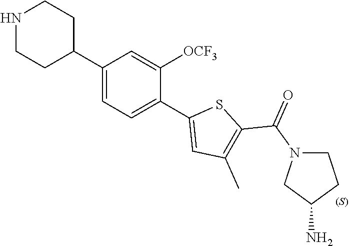 |
| 106 | 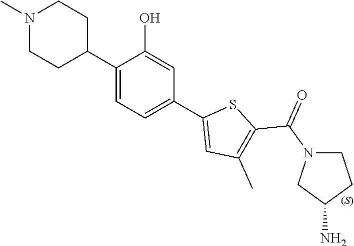 |
| 107 | 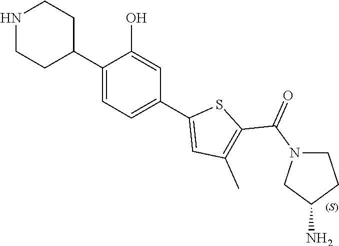 |
| 108 | 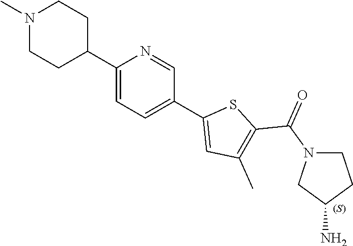 |
| 109 | 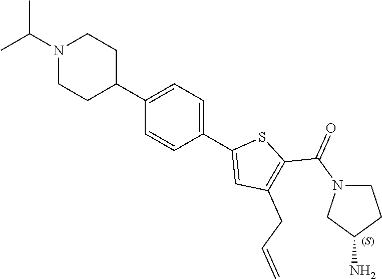 |
| 110 | 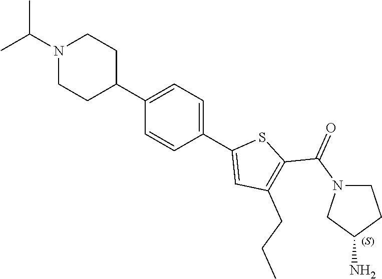 |
| 111 | 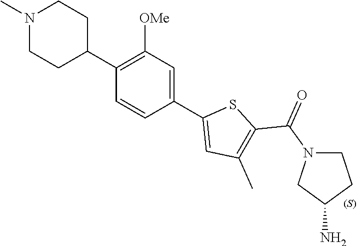 |
| 112 | 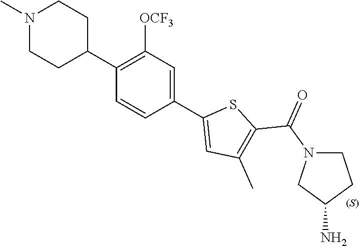 |
| 113 | 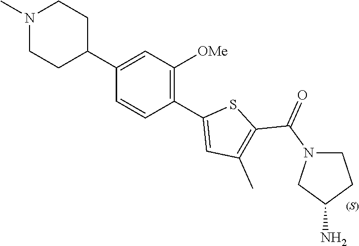 |
| 114 | 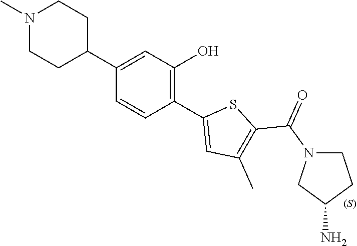 |
| 115 | 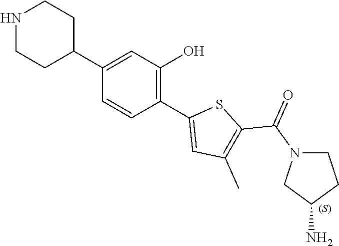 |
| 116 | 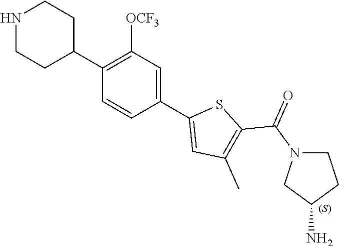 |
| 117 | 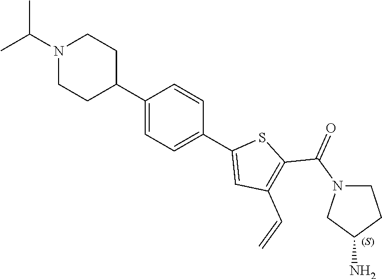 |
| 118 | 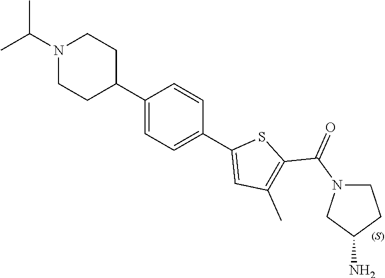 |
| 119 | 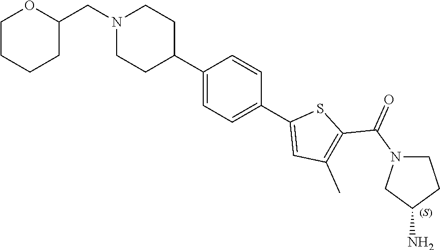 |
| 120 | 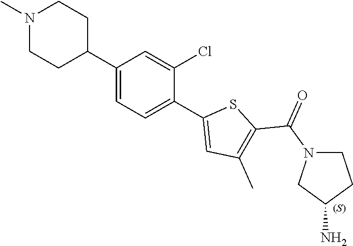 |
| 121 | 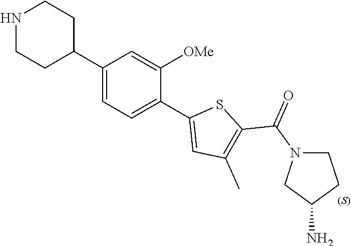 |
| 122 | 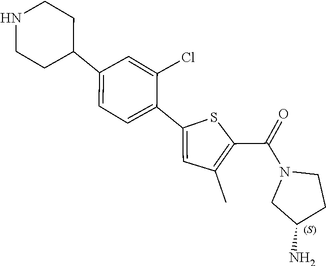 |
| 123 | 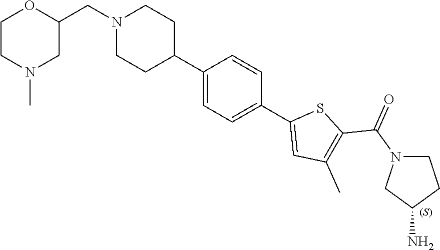 |
| 124 | 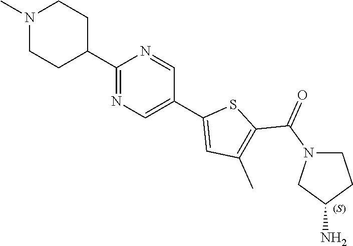 |
| 125 | 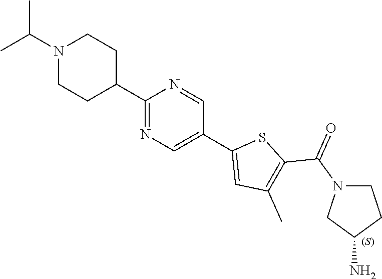 |
| 126 | 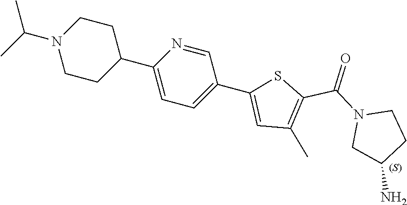 |
| 127 | 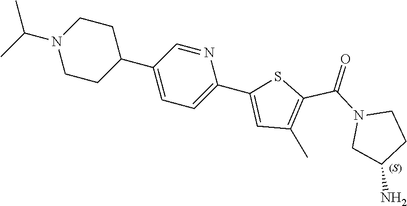 |
| 128 | 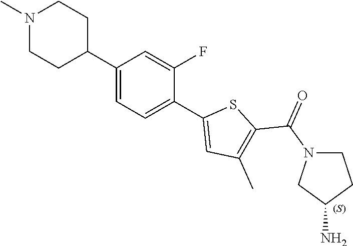 |
| 129 | 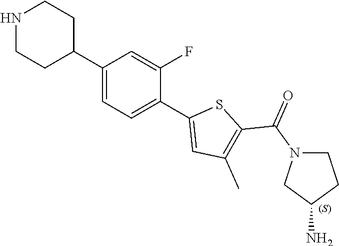 |
| 130 | 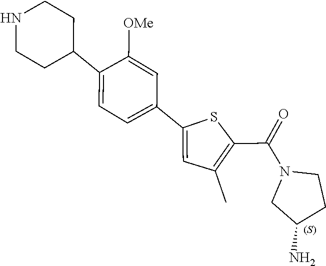 |
| 131 | 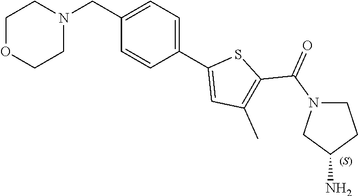 |
| 132 | 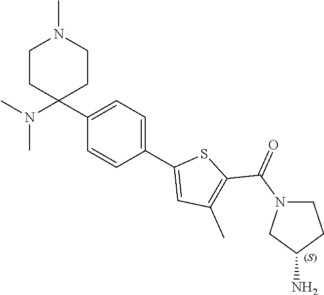 |
| 133 | 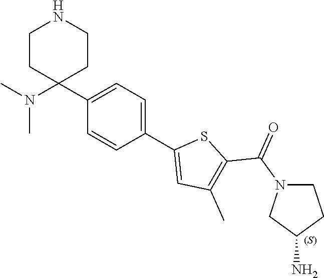 |
| 134 | 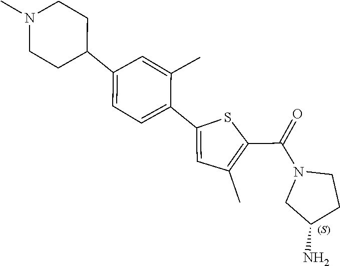 |
| 135 | 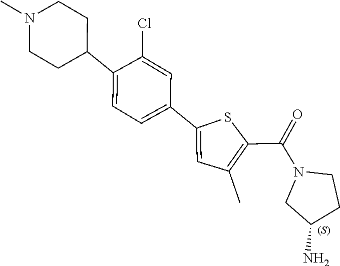 |
| 136 | 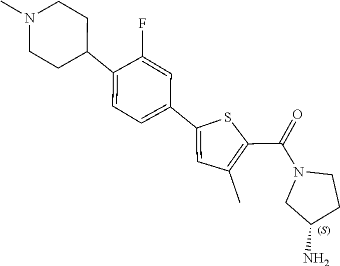 |
| 137 | 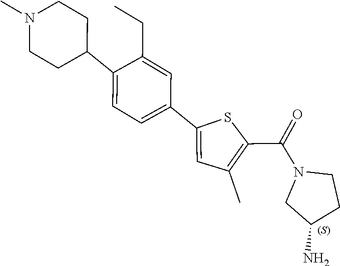 |
| 138 | 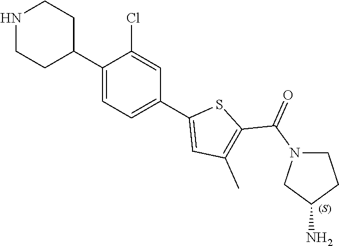 |
| 139 | 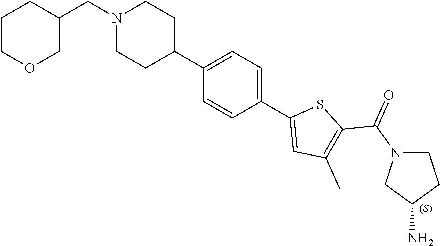 |
| 140 | 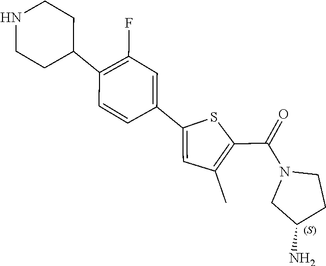 |
| 141 | 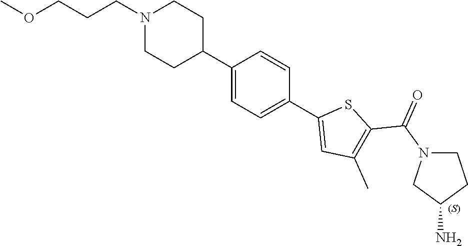 |
| 142 | 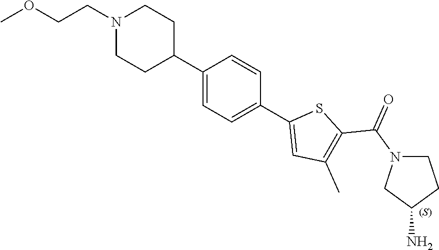 |
| 143 | 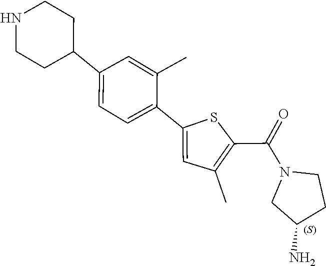 |
| 144 | 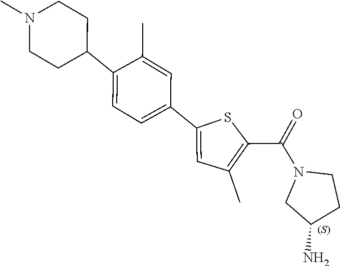 |
| 145 | 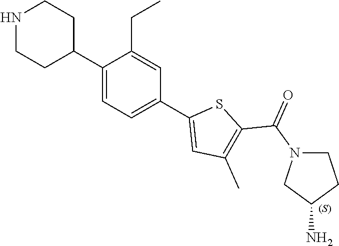 |
| 146 | 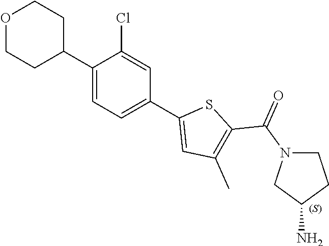 |
| 147 | 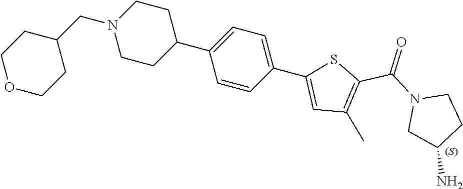 |
| 148 | 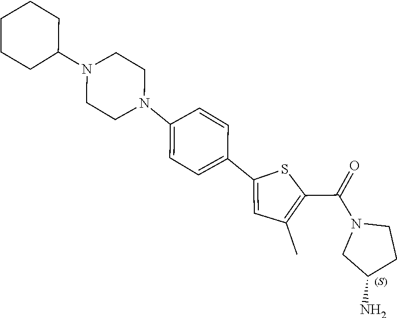 |
| 149 | 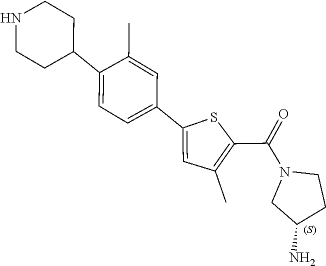 |
| 150 | 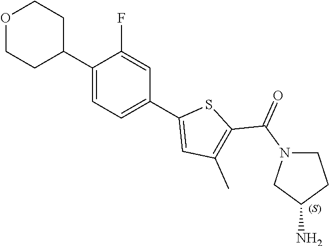 |
| 151 | 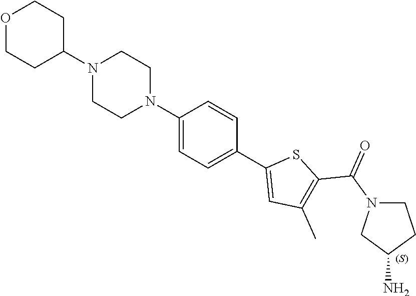 |
| 152 | 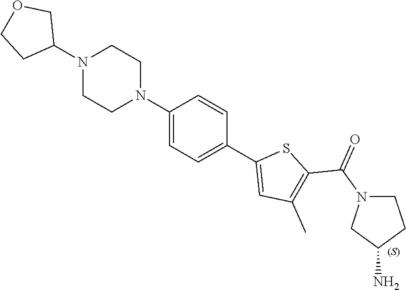 |
| 153 | 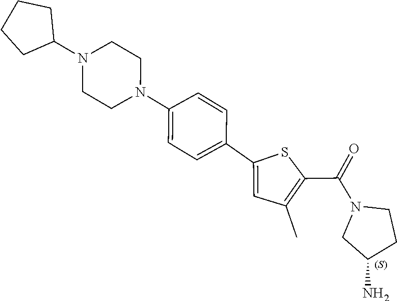 |
| 154 | 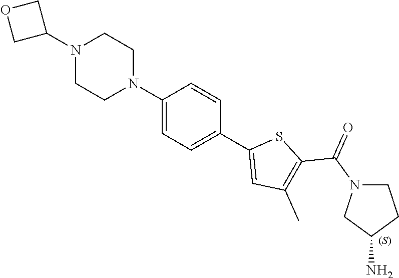 |
| 155 | 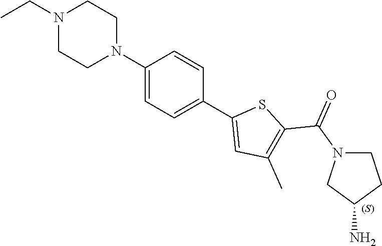 |
| 156 | 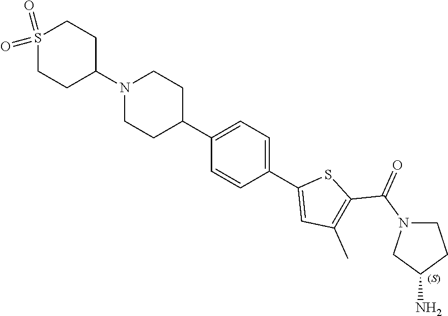 |
| 157 | 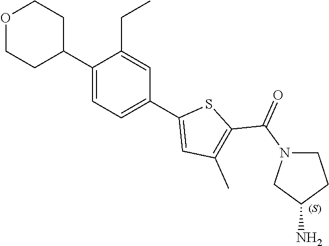 |
| 158 | 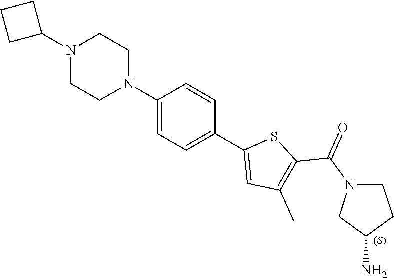 |
| 159 | 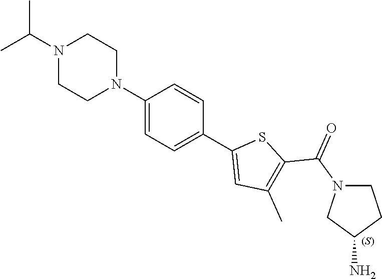 |
| 160 | 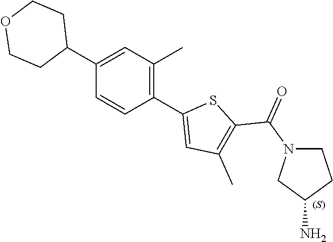 |
| 161 | 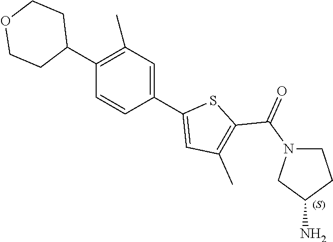 |
| 162 | 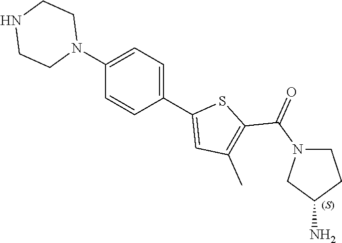 |
| 163 | 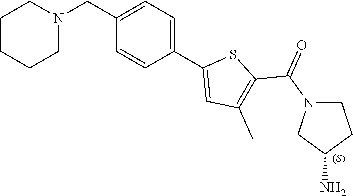 |
| 164 | 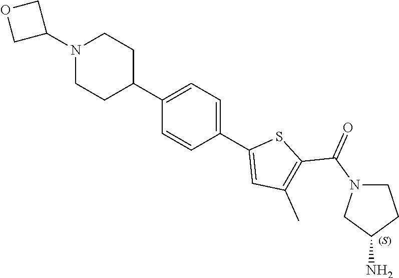 |
| 165 | 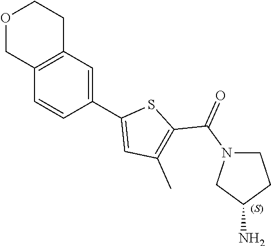 |
| 166 | 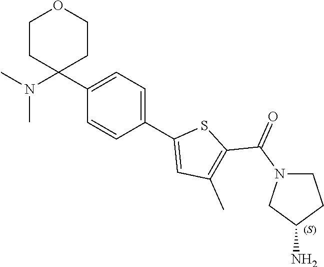 |
| 167 | 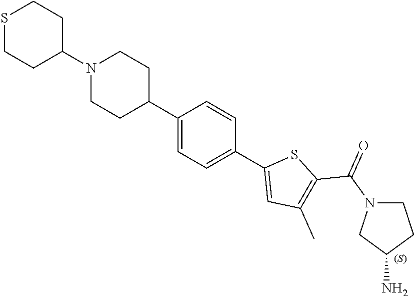 |
| 168 | 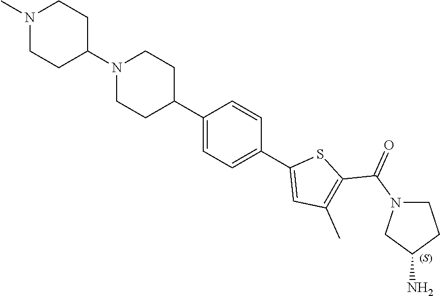 |
| 169 | 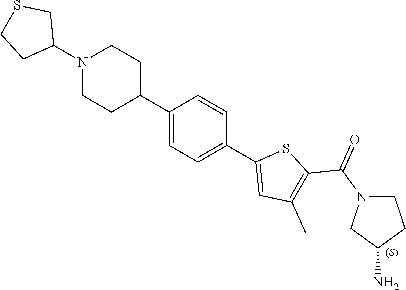 |
| 170 | 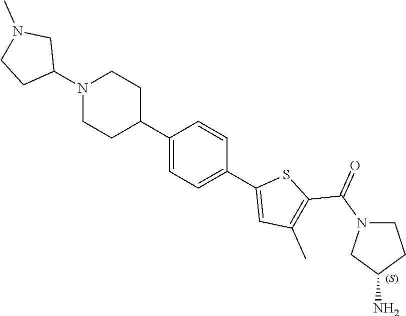 |
| 171 | 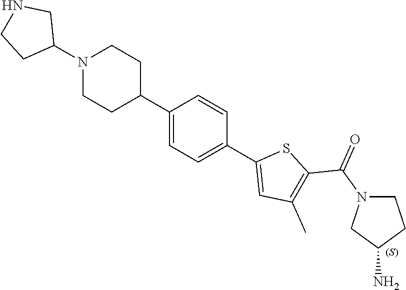 |
| 172 | 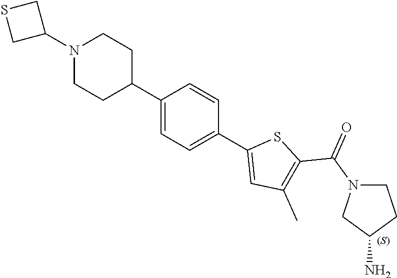 |
| 173 | 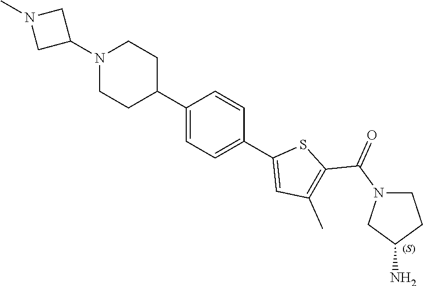 |
| 174 | 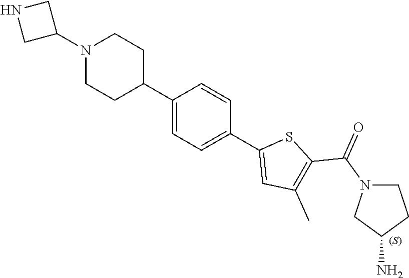 |
| 175 | 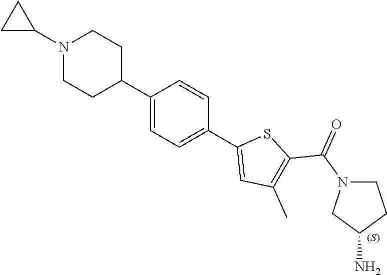 |
| 176 | 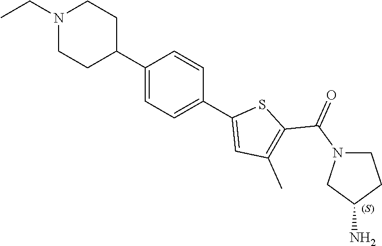 |
| 177 | 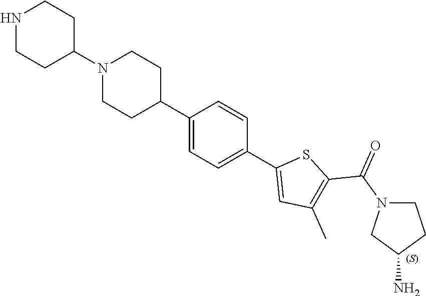 |
| 178 | 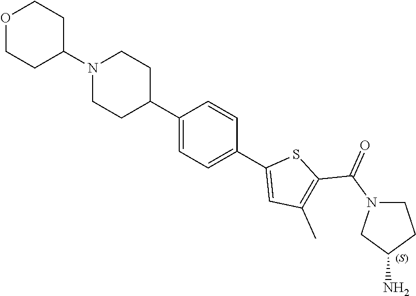 |
| 179 | 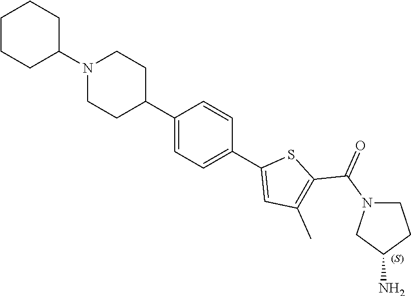 |
| 180 | 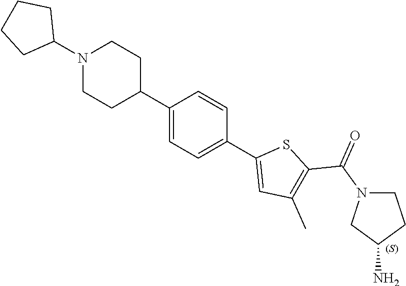 |
| 181 | 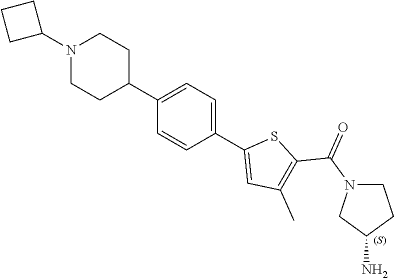 |
| 182 | 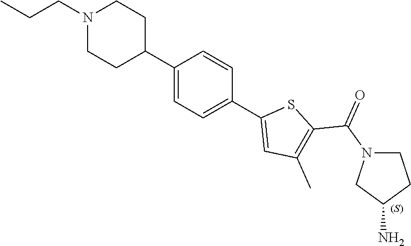 |
| 183 | 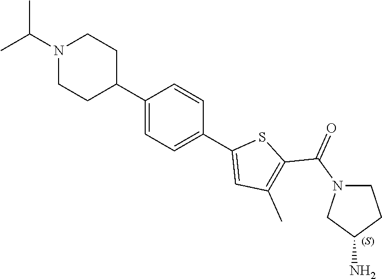 |
| 184 | 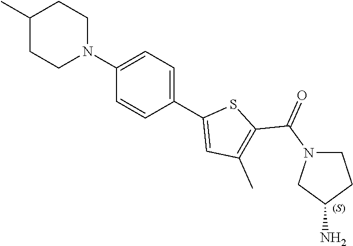 |
| 185 | 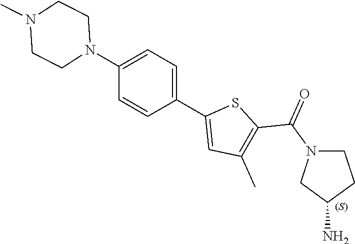 |
| 186 | 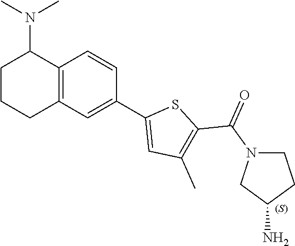 |
| 187 | 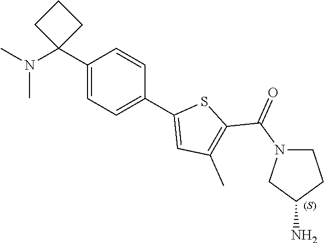 |
| 188 | 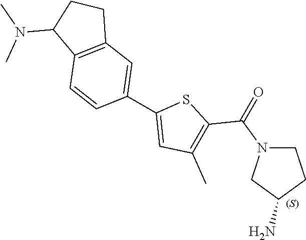 |
| 189 | 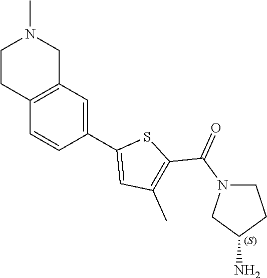 |
| 190 | 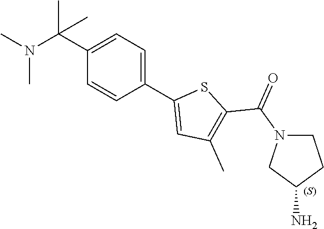 |
| 191 | 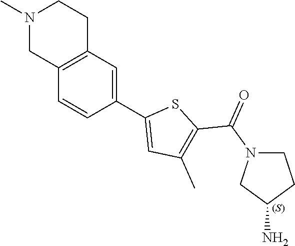 |
| 192 | 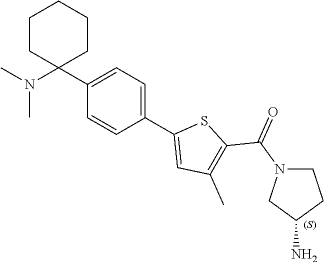 |
| 193 | 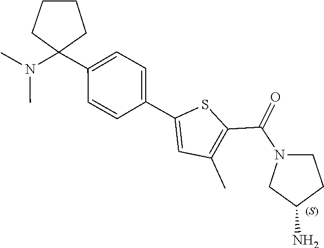 |
| 194 | 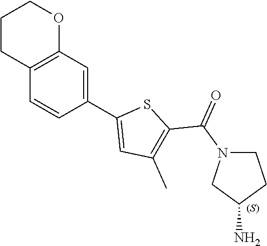 |
| 195 | 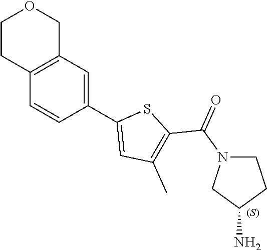 |
| 196 | 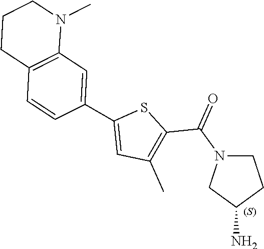 |
| 197 | 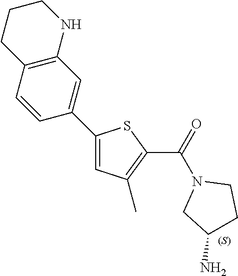 |
| 198 | 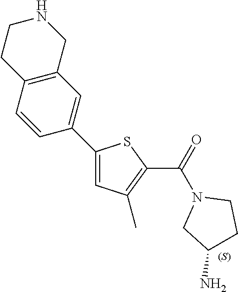 |
| 199 | 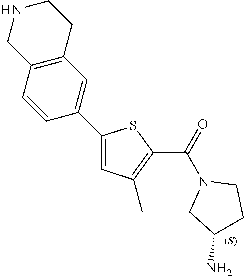 |
| 200 | 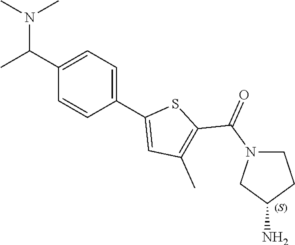 |
| 201 | 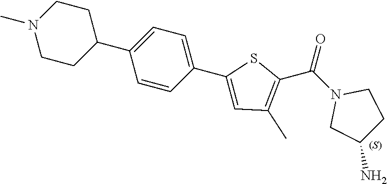 |
| 202 | 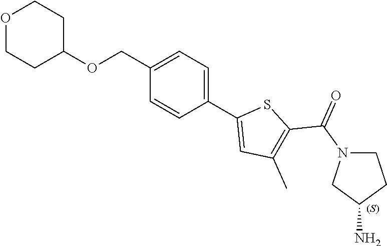 |
| 203 | 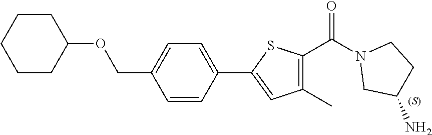 |
| 204 | 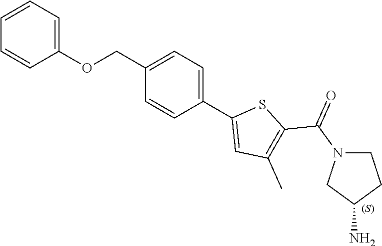 |
| 205 | 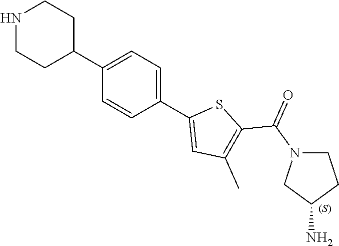 |
| 206 | 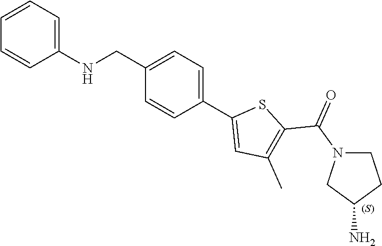 |
| 207 | 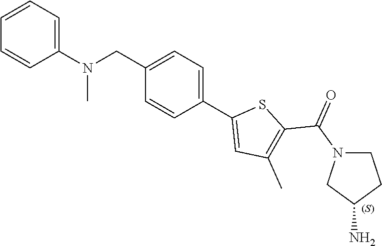 |
| 208 | 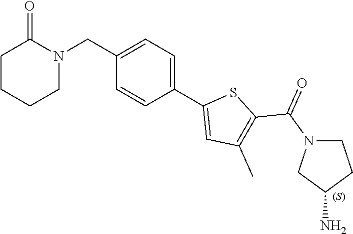 |
| 209 | 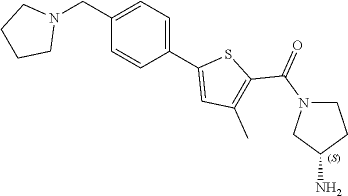 |
| 210 | 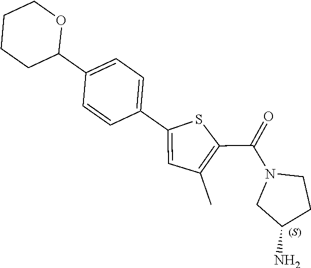 |
| 211 | 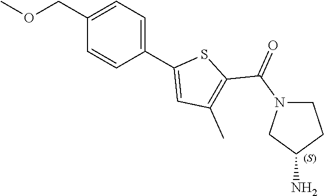 |
| 212 | 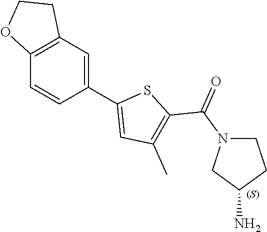 |
| 213 | 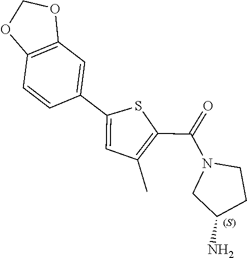 |
| 214 | 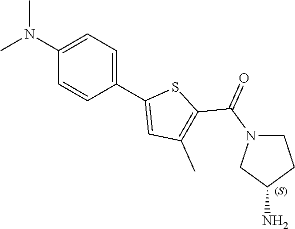 |
| 215 | 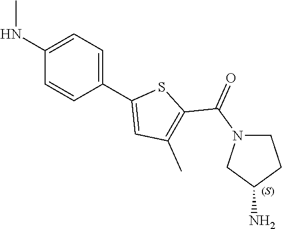 |
| 216 | 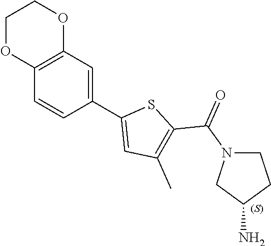 |
| 217 | 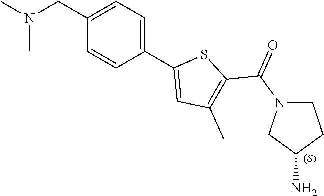 |
| 218 | 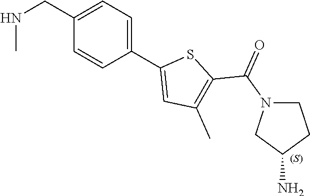 |
| 219 | 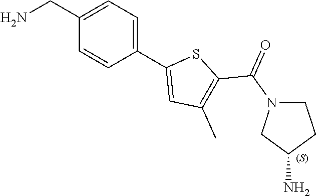 |
| 220 | 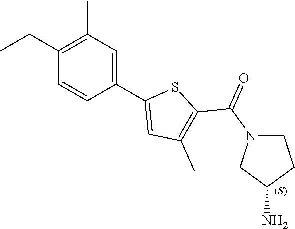 |
| 221 | 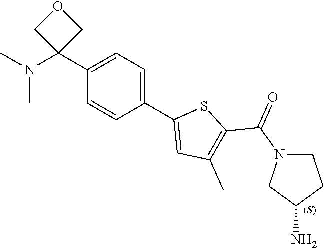 |
| 222 | 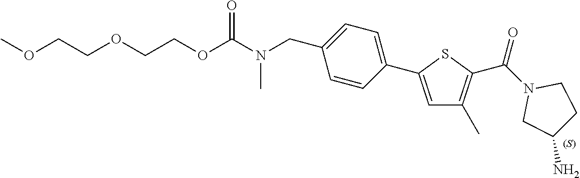 |
| 223 | 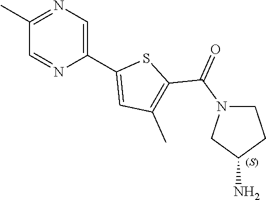 |
| 224 | 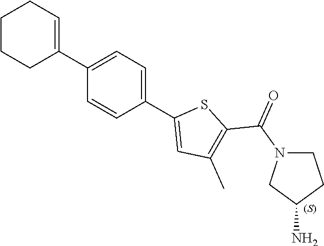 |
| 225 | 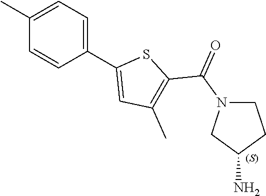 |
| 226 | 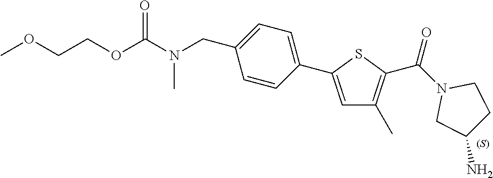 |
| 227 | 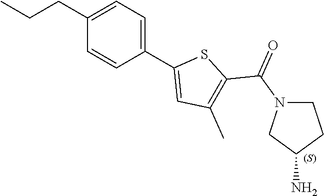 |
| 228 | 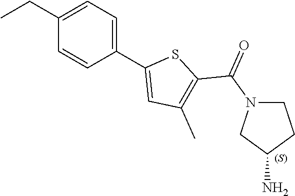 |
| 229 | 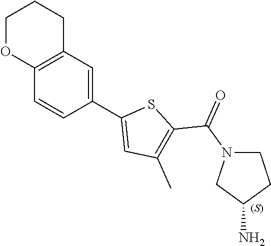 |
| 230 | 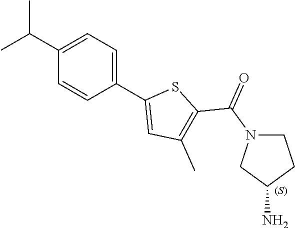 |
| 231 | 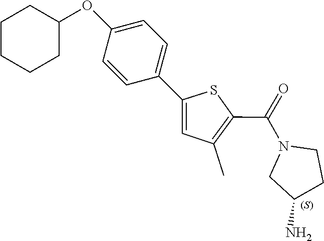 |
| 232 | 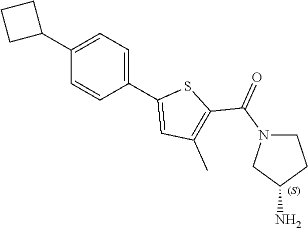 |
| 233 | 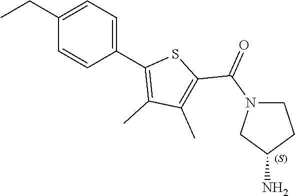 |
| 234 | 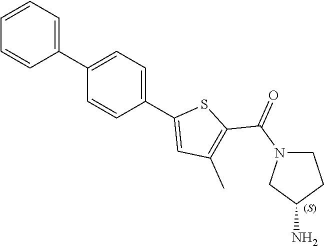 |
| 235 | 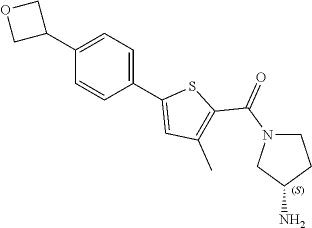 |
| 236 | 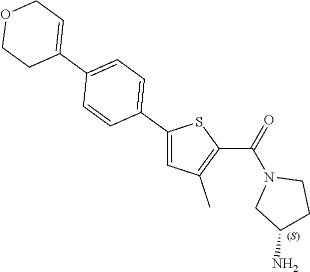 |
| 237 | 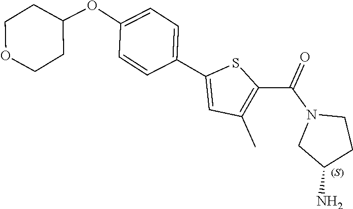 |
| 238 | 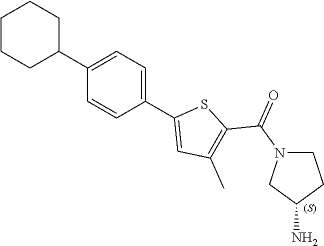 |
| 239 | 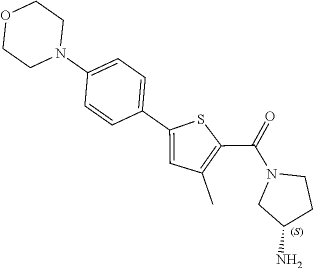 |
| 240 | 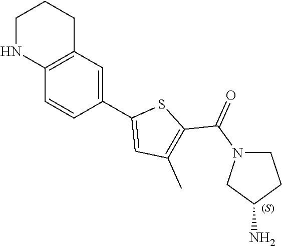 |
| 241 | 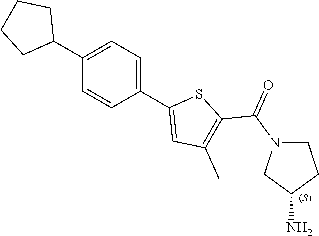 |
| 242 | 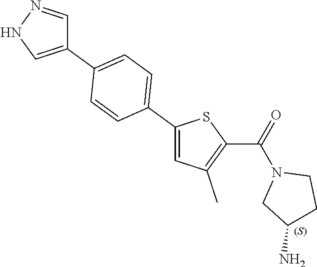 |
| 243 | 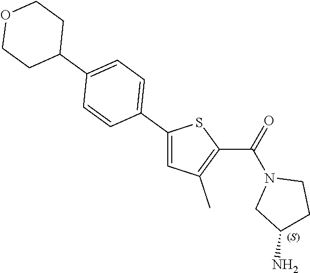 |
| 244 | 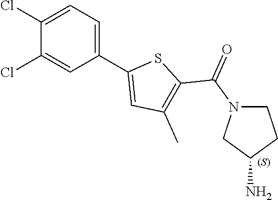 |
| 245 | 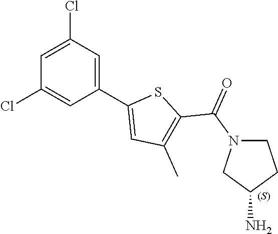 |
| 246 | 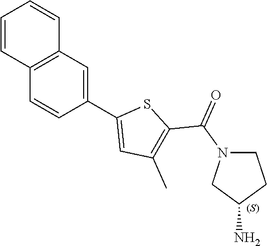 |
| 247 | 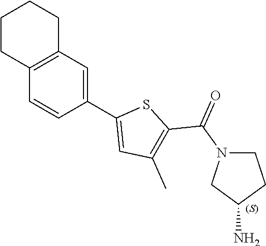 |
| 248 | 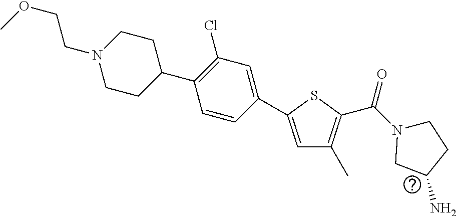 [SMILES: COCCN1CCC(c2ccc(-c3cc(C)c(C(=O)N4CC[C@H](N)C4)s3)cc2Cl)CC1] |
| 249 | 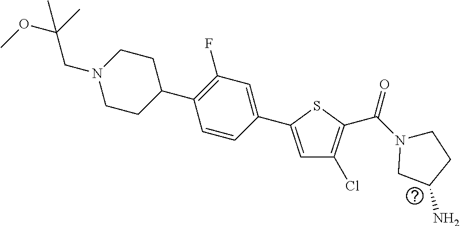 [SMILES: COC(C)(C)CN1CCC(c2ccc(-c3cc(Cl)c(C(=O)N4CC[C@H](N)C4)s3)cc2F)CC1] |
| 250 | 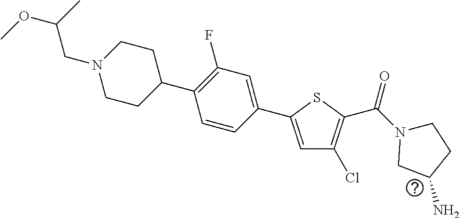 [SMILES: COC(C)CN1CCC(c2ccc(-c3cc(Cl)c(C(=O)N4CC[C@H](N)C4)s3)cc2F)CC1] |
| 251 | 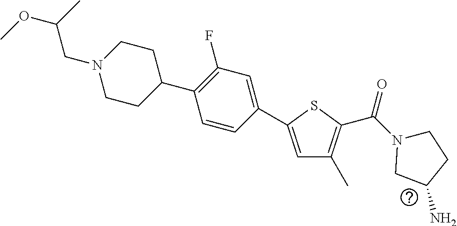 [SMILES: COC(C)CN1CCC(c2ccc(-c3cc(C)c(C(=O)N4CC[C@H](N)C4)s3)cc2F)CC1] |
| 252 | 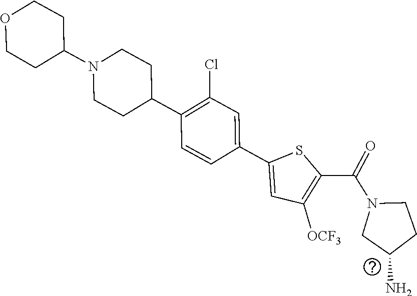 [SMILES: N[C@H]1CCN(C(=O)c2sc(-c3ccc(C4CCN(C5CCOCC5)CC4)c(Cl)c3)cc2OC(F)(F)F)C1] |
| 253 | 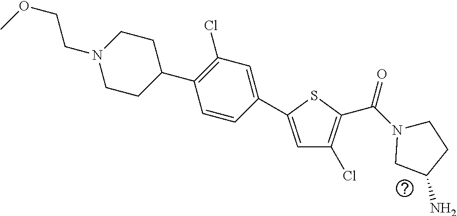 [SMILES: COCCN1CCC(c2ccc(-c3cc(Cl)c(C(=O)N4CC[C@H](N)C4)s3)cc2Cl)CC1] |
| 254 | 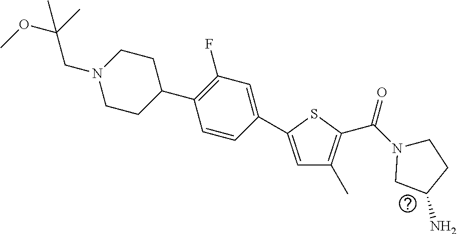 [SMILES: COC(C)(C)CN1CCC(c2ccc(-c3cc(C)c(C(=O)N4CC[C@H](N)C4)s3)cc2F)CC1] |
| 255 | 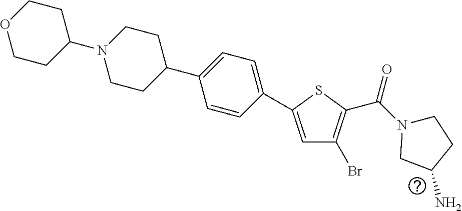 [SMILES: N[C@H]1CCN(C(=O)c2sc(-c3ccc(C4CCN(C5CCOCC5)CC4)cc3)cc2Br)C1] |
| 256 | 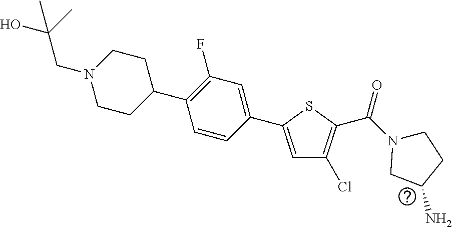 [SMILES: CC(C)(O)CN1CCC(c2ccc(-c3cc(Cl)c(C(=O)N4CC[C@H](N)C4)s3)cc2F)CC1] |
| 257 | 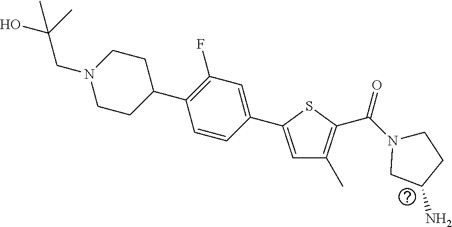 [SMILES: Cc1cc(-c2ccc(C3CCN(CC(C)(C)O)CC3)c(F)c2)sc1C(=O)N1CC[C@H](N)C1] |
| 258 | 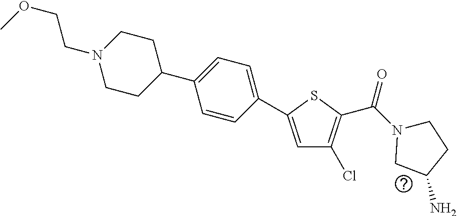 [SMILES: COCCN1CCC(c2ccc(-c3cc(Cl)c(C(=O)N4CC[C@H](N)C4)s3)cc2)CC1] |
| 259 | 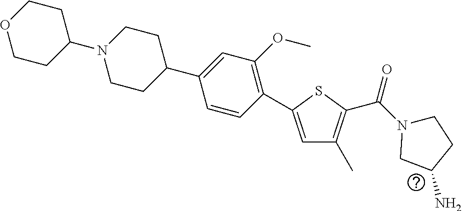 [SMILES: COc1cc(C2CCN(C3CCOCC3)CC2)ccc1-c1cc(C)c(C(=O)N2CC[C@H](N)C2)s1] |
| 260 | 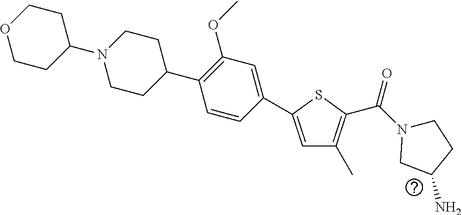 [SMILES: COc1cc(-c2cc(C)c(C(=O)N3CC[C@H](N)C3)s2)ccc1C1CCN(C2CCOCC2)CC1] |
| 261 | 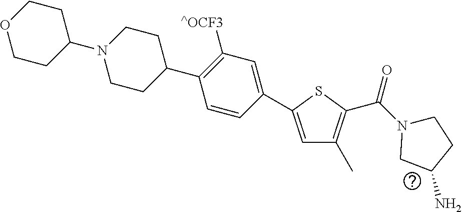 |
| 262 | 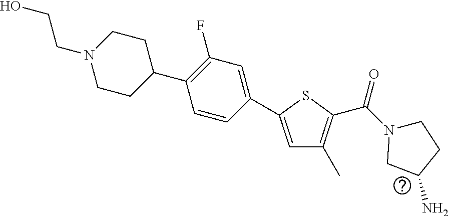 [SMILES: Cc1cc(-c2ccc(C3CCN(CCO)CC3)c(F)c2)sc1C(=O)N1CC[C@H](N)C1] |
| 263 | 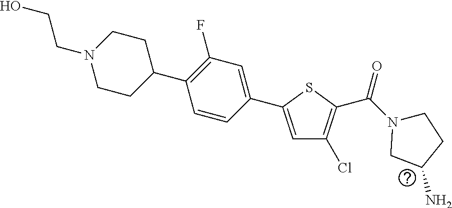 [SMILES: N[C@H]1CCN(C(=O)c2sc(-c3ccc(C4CCN(CCO)CC4)c(F)c3)cc2Cl)C1] |
| 264 | 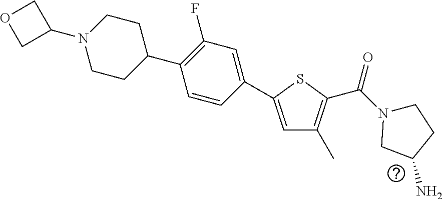 [SMILES: Cc1cc(-c2ccc(C3CCN(C4COC4)CC3)c(F)c2)sc1C(=O)N1CC[C@H](N)C1] |
| 265 | 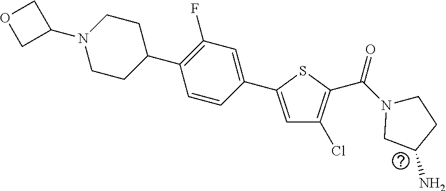 [SMILES: N[C@H]1CCN(C(=O)c2sc(-c3ccc(C4CCN(C5COC5)CC4)c(F)c3)cc2Cl)C1] |
| 266 | 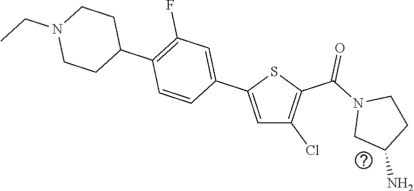 [SMILES: CCN1CCC(c2ccc(-c3cc(Cl)c(C(=O)N4CC[C@H](N)C4)s3)cc2F)CC1] |
| 267 | 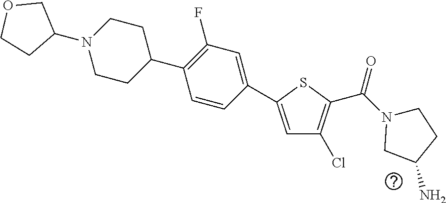 [SMILES: N[C@H]1CCN(C(=O)c2sc(-c3ccc(C4CCN(C5CCOC5)CC4)c(F)c3)cc2Cl)C1] |
| 268 | 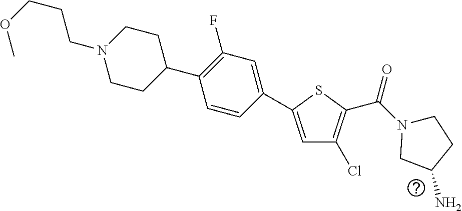 [SMILES: COCCCN1CCC(c2ccc(-c3cc(Cl)c(C(=O)N4CC[C@H](N)C4)s3)cc2F)CC1] |
| 269 | 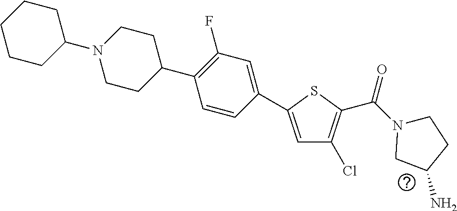 [SMILES: N[C@H]1CCN(C(=O)c2sc(-c3ccc(C4CCN(C5CCCCC5)CC4)c(F)c3)cc2Cl)C1] |
| 270 | 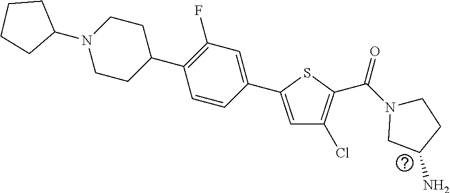 [SMILES: N[C@H]1CCN(C(=O)c2sc(-c3ccc(C4CCN(C5CCCC5)CC4)c(F)c3)cc2Cl)C1] |
| 271 | 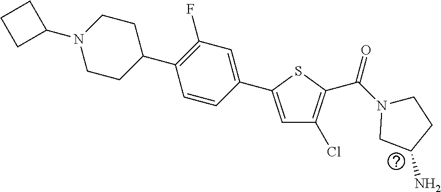 [SMILES: N[C@H]1CCN(C(=O)c2sc(-c3ccc(C4CCN(C5CCC5)CC4)c(F)c3)cc2Cl)C1] |
| 272 | 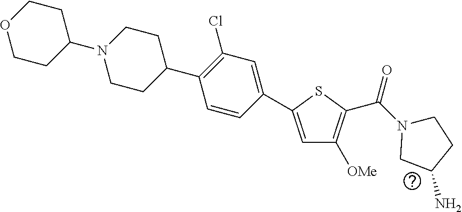 [SMILES: COc1cc(-c2ccc(C3CCN(C4CCOCC4)CC3)c(Cl)c2)sc1C(=O)N1CC[C@H](N)C1] |
| 273 | 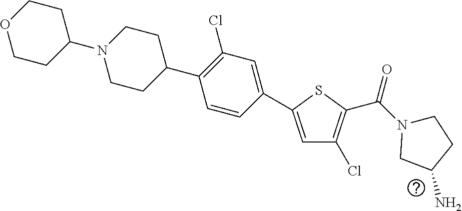 [SMILES: N[C@H]1CCN(C(=O)c2sc(-c3ccc(C4CCN(C5CCOCC5)CC4)c(Cl)c3)cc2Cl)C1] |
| 274 | 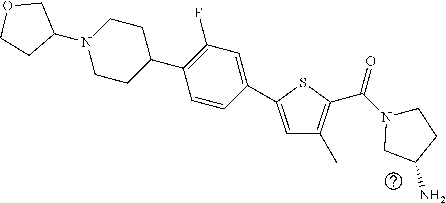 [SMILES: Cc1cc(-c2ccc(C3CCN(C4CCOC4)CC3)c(F)c2)sc1C(=O)N1CC[C@H](N)C1] |
| 275 | 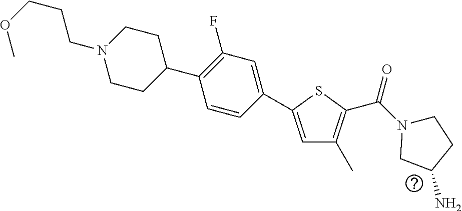 [SMILES: COCCCN1CCC(c2ccc(-c3cc(C)c(C(=O)N4CC[C@H](N)C4)s3)cc2F)CC1] |
| 276 | 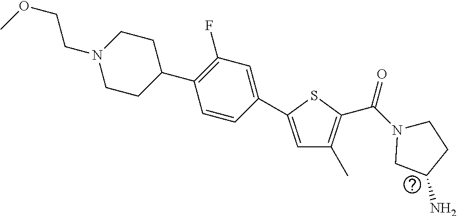 [SMILES: COCCN1CCC(c2ccc(-c3cc(C)c(C(=O)N4CC[C@H](N)C4)s3)cc2F)CC1] |
| 277 | 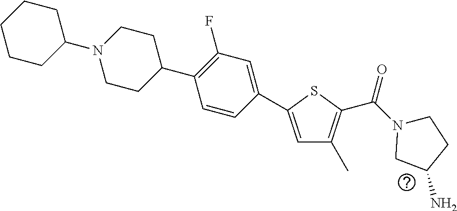 [SMILES: Cc1cc(-c2ccc(C3CCN(C4CCCCC4)CC3)c(F)c2)sc1C(=O)N1CC[C@H](N)C1] |
| 278 | 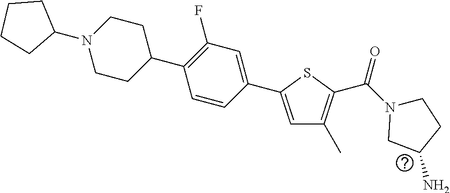 [SMILES: Cc1cc(-c2ccc(C3CCN(C4CCCC4)CC3)c(F)c2)sc1C(=O)N1CC[C@H](N)C1] |
| 279 | 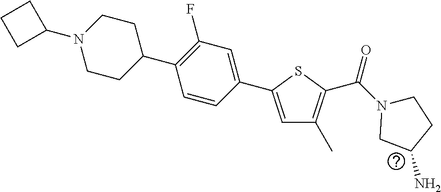 [SMILES: Cc1cc(-c2ccc(C3CCN(C4CCC4)CC3)c(F)c2)sc1C(=O)N1CC[C@H](N)C1] |
| 280 | 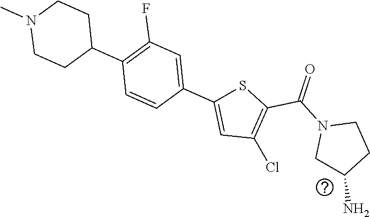 [SMILES: CN1CCC(c2ccc(-c3cc(Cl)c(C(=O)N4CC[C@H](N)C4)s3)cc2F)CC1] |
| 281 | 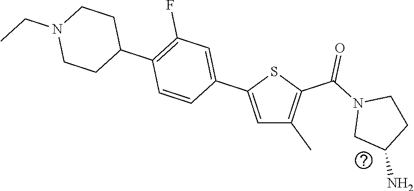 [SMILES: CCN1CCC(c2ccc(-c3cc(C)c(C(=O)N4CC[C@H](N)C4)s3)cc2F)CC1] |
| 282 | 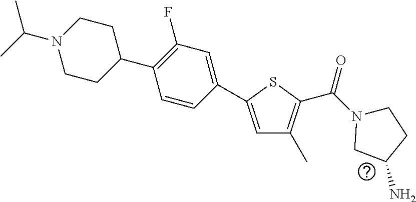 [SMILES: Cc1cc(-c2ccc(C3CCN(C(C)C)CC3)c(F)c2)sc1C(=O)N1CC[C@H](N)C1] |
| 283 | 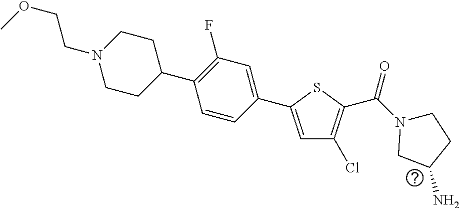 [SMILES: COCCN1CCC(c2ccc(-c3cc(Cl)c(C(=O)N4CC[C@H](N)C4)s3)cc2F)CC1] |
| 284 | 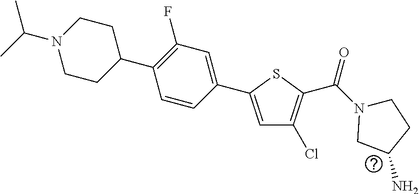 [SMILES: CC(C)N1CCC(c2ccc(-c3cc(Cl)c(C(=O)N4CC[C@H](N)C4)s3)cc2F)CC1] |
| 285 | 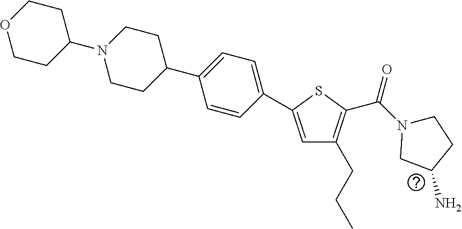 [SMILES: CCCc1cc(-c2ccc(C3CCN(C4CCOCC4)CC3)cc2)sc1C(=O)N1CC[C@H](N)C1] |
| 286 | 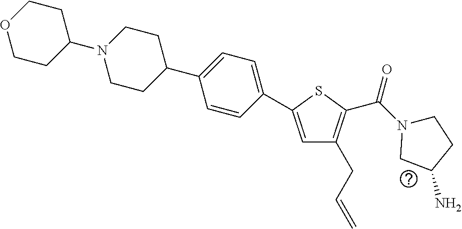 [SMILES: C=CCc1cc(-c2ccc(C3CCN(C4CCOCC4)CC3)cc2)sc1C(=O)N1CC[C@H](N)C1] |
| 287 | 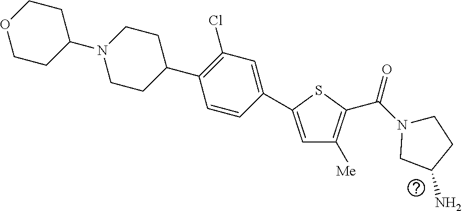 [SMILES: Cc1cc(-c2ccc(C3CCN(C4CCOCC4)CC3)c(Cl)c2)sc1C(=O)N1CC[C@H](N)C1] |
| 288 | 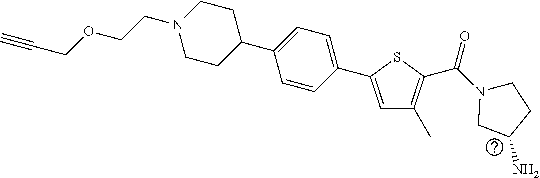 [SMILES: C#CCOCCN1CCC(c2ccc(-c3cc(C)c(C(=O)N4CC[C@H](N)C4)s3)cc2)CC1] |
| 289 | 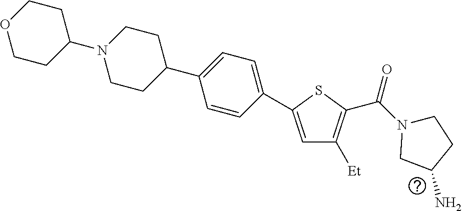 [SMILES: CCc1cc(-c2ccc(C3CCN(C4CCOCC4)CC3)cc2)sc1C(=O)N1CC[C@H](N)C1] |
| 290 | 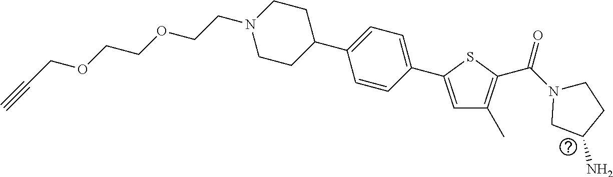 [SMILES: C#CCOCCOCCN1CCC(c2ccc(-c3cc(C)c(C(=O)N4CC[C@H](N)C4)s3)cc2)CC1] |
| 291 | 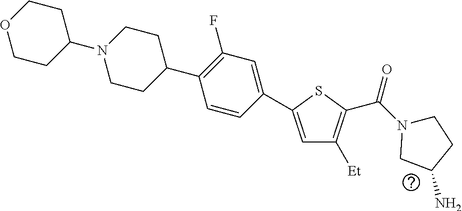 [SMILES: CCc1cc(-c2ccc(C3CCN(C4CCOCC4)CC3)c(F)c2)sc1C(=O)N1CC[C@H](N)C1] |
| 292 | 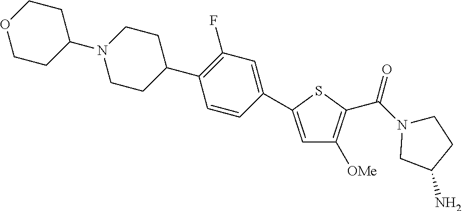 [SMILES: COc1cc(-c2ccc(C3CCN(C4CCOCC4)CC3)c(F)c2)sc1C(=O)N1CC[C@H](N)C1] |
| 293 | 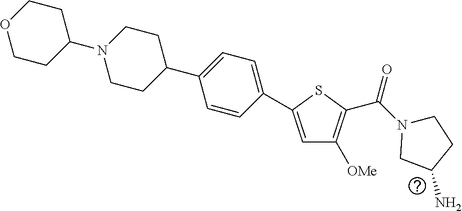 [SMILES: COc1cc(-c2ccc(C3CCN(C4CCOCC4)CC3)cc2)sc1C(=O)N1CC[C@H](N)C1] |
| 294 | 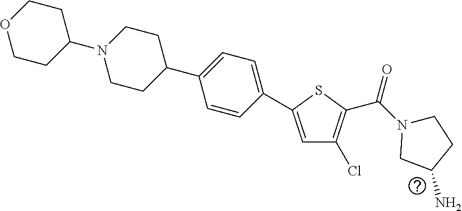 [SMILES: N[C@H]1CCN(C(=O)c2sc(-c3ccc(C4CCN(C5CCOCC5)CC4)cc3)cc2Cl)C1] |
| 295 | 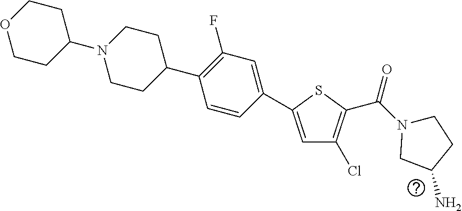 [SMILES: N[C@H]1CCN(C(=O)c2sc(-c3ccc(C4CCN(C5CCOCC5)CC4)c(F)c3)cc2Cl)C1] |
| 296 | 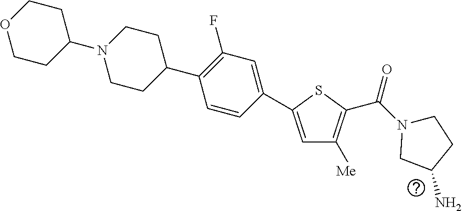 [SMILES: Cc1cc(-c2ccc(C3CCN(C4CCOCC4)CC3)c(F)c2)sc1C(=O)N1CC[C@H](N)C1] |

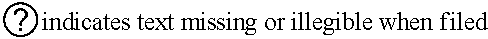

### Methods of Treatment and Use

In the following methods, use of a compound disclosed herein (e.g., a compound of Formula (I), (II), (III), or (IV), or subformulas thereof, or a compound of Table 1, or a pharmaceutically acceptable salt thereof) can also refer to use of a pharmaceutical composition including a compound disclosed herein (e.g., a compound of Formula (I), (II), (III), or (IV), or subformulas thereof, or a compound of Table 1, or a pharmaceutically acceptable salt thereof). In an aspect, the disclosure provides methods for treating a neurodegenerative disease or disorder, a musculoskeletal disease or disorder, a cancer, an ophthalmological disease or disorder (e.g., a retinal disease or disorder), or a viral infection in a subject in need thereof, the methods generally comprise administering to a subject in need thereof an effective amount of a compound disclosed herein (e.g., a compound of Formula (I), (II), (III), or (IV), or subformulas thereof, or a compound of Table 1, or a pharmaceutically acceptable salt thereof).

In some embodiments, the subject is suffering from a neurodegenerative disease or disorder. In some embodiments, the subject is suffering from a musculoskeletal disease or disorder. In some embodiments, the subject is suffering from a cancer. In some embodiments, the subject is suffering from an ophthalmological disease or disorder (e.g., a retinal disease or disorder). In some embodiments, the subject is suffering from a viral infection.

In some embodiments, the disclosure provides methods for treating a neurodegenerative disease or disorder, the methods comprise administering to a subject in need thereof an effective amount of a compound disclosed herein (e.g., a compound of Formula (I), (II), (III), or (IV), or subformulas thereof, or a compound of Table 1, or a pharmaceutically acceptable salt thereof).

In some embodiments, the disclosure provides methods for treating a musculoskeletal disease or disorder, the methods comprise administering to a subject in need thereof an effective amount of a compound disclosed herein (e.g., a compound of Formula (I), (II), (III), or (IV), or subformulas thereof, or a compound of Table 1, or a pharmaceutically acceptable salt thereof).

In some embodiments, the disclosure provides methods for treating a cancer, the methods comprise administering to a subject in need thereof an effective amount of a compound disclosed herein (e.g., a compound of Formula (I), (II), (III), or (IV), or subformulas thereof, or a compound of Table 1, or a pharmaceutically acceptable salt thereof).

In some embodiments, the disclosure provides methods for treating an ophthalmological disease or disorder (e.g., a retinal disease or disorder), the methods comprise administering to a subject in need thereof an effective amount of a compound disclosed herein (e.g., a compound of Formula (I), (II), (III), or (IV), or subformulas thereof, or a compound of Table 1, or a pharmaceutically acceptable salt thereof).

In some embodiments, the disclosure provides methods for treating a viral infection, the methods comprise administering to a subject in need thereof an effective amount of a compound disclosed herein (e.g., a compound of Formula (I), (II), (III), or (IV), or subformulas thereof, or a compound of Table 1, or a pharmaceutically acceptable salt thereof).

In some embodiments, the methods further comprise the step of diagnosing the subject with a neurodegenerative disease or disorder, a musculoskeletal disease or disorder, a cancer, an ophthalmological disease or disorder (e.g., a retinal disease or disorder), or a viral infection prior to administration of a compound disclosed herein (e.g., a compound of Formula (I), (II), (III), or (IV), or subformulas thereof, or a compound of Table 1, or a pharmaceutically acceptable salt thereof).

In some embodiments, the methods further comprise the step of diagnosing the subject with a neurodegenerative disease or disorder prior to administration of a compound disclosed herein (e.g., a compound of Formula (I), (II), (III), or (IV), or subformulas thereof, or a compound of Table 1, or a pharmaceutically acceptable salt thereof).

In some embodiments, the methods further comprise the step of diagnosing the subject with a neurodegenerative disease or disorder prior to administration of a compound disclosed herein (e.g., a compound of Formula (I), (II), (III), or (IV), or subformulas thereof, or a compound of Table 1, or a pharmaceutically acceptable salt thereof).

In some embodiments, the methods further comprise the step of diagnosing the subject with a musculoskeletal disease or disorder prior to administration of a compound disclosed herein (e.g., a compound of Formula (I), (II), (III), or (IV), or subformulas thereof, or a compound of Table 1, or a pharmaceutically acceptable salt thereof).

In some embodiments, the methods further comprise the step of diagnosing the subject with a cancer prior to administration of a compound disclosed herein (e.g., a compound of Formula (I), (II), (III), or (IV), or subformulas thereof, or a compound of Table 1, or a pharmaceutically acceptable salt thereof).

In some embodiments, the methods further comprise the step of diagnosing the subject with an ophthalmological disease or disorder (e.g., a retinal disease or disorder) prior to administration of a compound disclosed herein (e.g., a compound of Formula (I), (II), (III), or (IV), or subformulas thereof, or a compound of Table 1, or a pharmaceutically acceptable salt thereof).

In some embodiments, the methods further comprise the step of diagnosing the subject with a viral infection prior to administration of a compound disclosed herein (e.g., a compound of Formula (I), (II), (III), or (IV), or subformulas thereof, or a compound of Table 1, or a pharmaceutically acceptable salt thereof).

In another aspect, the disclosure provides methods of diagnosing a neurodegenerative disease, a musculoskeletal disease, a cancer, an ophthalmological disease (e.g., a retinal disease), or a viral infection in a subject, the methods generally comprise administering a compound disclosed herein (e.g., a compound of Formula (I), (II), (III), or (IV), or subformulas thereof, or a compound of Table 1, or a pharmaceutically acceptable salt thereof) to the subject.

In some embodiments, the disclosure provides methods of diagnosing a neurodegenerative disease in a subject, the method comprising administering a compound disclosed herein (e.g., a compound of Formula (I), (II), (III), or (IV), or subformulas thereof, or a compound of Table 1, or a pharmaceutically acceptable salt thereof) to the subject.

In some embodiments, the disclosure provides methods of diagnosing a musculoskeletal disease in a subject, the method comprising administering a compound disclosed herein (e.g., a compound of Formula (I), (II), (III), or (IV), or subformulas thereof, or a compound of Table 1, or a pharmaceutically acceptable salt thereof).

In some embodiments, the disclosure provides methods of diagnosing a cancer in a subject, the method comprising administering a compound disclosed herein (e.g., a compound of Formula (I), (II), (III), or (IV), or subformulas thereof, or a compound of Table 1, or a pharmaceutically acceptable salt thereof).

In some embodiments, the disclosure provides methods of diagnosing an ophthalmological disease (e.g., a retinal disease) in a subject, the method comprising administering a compound disclosed herein (e.g., a compound of Formula (I), (II), (III), or (IV), or subformulas thereof, or a compound of Table 1, or a pharmaceutically acceptable salt thereof).

In some embodiments, the disclosure provides methods of diagnosing a viral infection in a subject, the method comprising administering a compound disclosed herein (e.g., a compound of Formula (I), (II), (III), or (IV), or subformulas thereof, or a compound of Table 1, or a pharmaceutically acceptable salt thereof).

In some embodiments, the subject is a mammal. In some embodiments, the subject is a nematode. In some embodiments, the subject is human.

### Stress Granules and TDP-43

By comprising stress granules is meant that number of stress granules in a cell in the subject is changed relative to a control or healthy subject or relative to before onset of said disease or disorder. Exemplary diseases and disorders pathology of which incorporate stress granules include, but are not limited to, neurodegenerative diseases, musculoskeletal diseases, cancers, ophthalmological diseases (e.g., retinal diseases), and viral infections.

TDP-43 and other RNA-binding proteins function in both the nucleus and cytoplasm to process mRNA, e.g., by splicing mRNA, cleaving mRNA introns, cleaving untranslated regions of mRNA or modifying protein translation at the synapse, axon, dendrite or soma. Therefore, targeting other proteins that function in an analogous manner to TDP-43 or by processing mRNA may also be beneficial to prevent and treat neurodegeneration resulting from disease. For instance, the fragile X mental retardation 1 (FMRP) protein is essential for normal cognitive development. The signaling systems that affect TDP-43 function might also affect this protein, thus improving cognitive function. This can be particularly important at the synapse where neurons communicate. Without being bound by a theory, the signaling systems that compounds of Formula (I), (II), or (III) target may also modify these processes, which play a role in neurodegeneration or mental health illnesses (e.g., schizophrenia).

The cellular stress response follows a U-shaped curve. Overinduction of this pathway, such as observed in many neurodegenerative diseases, can be harmful for cells. However, a decreased stimulation of this pathway can also be harmful for cells, e.g., in the case of an acute stress, such as a stroke. Thus, the appropriate action for some diseases is the inhibition of stress granule formation, while for other diseases, stimulation of stress granule formation is beneficial.

In some embodiments, the TDP-43 protein in a stress granule may be wild-type or a mutant form of TDP-43. In some embodiments, the mutant form of TDP-43 comprises an amino acid addition, deletion, or substitution, e.g., relative to the wild type sequence of TDP-43. In some embodiments, the mutant form of TDP-43 comprises an amino acid substitution relative to the wild type sequence (e.g., a G294A, A135T, Q331K, or Q343R substitution). In some embodiments, the TDP-43 protein in a stress granule comprises a post-translational modification (e.g., phosphorylation of an amino acid side chain (e.g., T103, S104, S409, or S410)).

In some embodiments, the pathology of the neurodegenerative disease or disorder, the musculoskeletal disease or disorder, the cancer, the ophthalmological disease or disorder, or the viral infection comprises stress granules. In some embodiments, the pathology of the neurodegenerative disease, the musculoskeletal disease or disorder, the cancer, the ophthalmological disease or disorder, or the viral infection comprises TDP-43 inclusions.

In some embodiments, administering a compound disclosed herein (e.g., a compound of Formula (I), (II), (III), or (IV), or subformulas thereof, or a compound of Table 1, or a pharmaceutically acceptable salt thereof) to the subject inhibits the formation of a stress granule. In some embodiments, administering a compound disclosed herein (e.g., a compound of Formula (I), (II), (III), or (IV), or subformulas thereof, or a compound of Table 1, or a pharmaceutically acceptable salt thereof) to the subject inhibits the formation of a stress granule by at least about 10%, at least about 20%, at least about 30%, at least about 40%, at least about 50%, at least about 60%, at least about 70%, at least about 80%, at least about 90%, at least about 95%, or 100% (i.e., complete inhibition) relative to a control (e.g., a baseline of the formation of a stress granule in the subject; the formation of a stress granule in the subject prior to administering a compound disclosed herein; the formation of a stress granule in a subject not receiving a compound disclosed herein; or the formation of a stress granule in a subject receiving a placebo).

In some embodiments, administering a compound disclosed herein (e.g., a compound of Formula (I), (II), (III), or (IV), or subformulas thereof, or a compound of Table 1, or a pharmaceutically acceptable salt thereof) to a subject in need thereof disaggregates a stress granule. In some embodiments, administering a compound disclosed herein (e.g., a compound of Formula (I), (II), or (III) or subformulas thereof, or a compound of Table 1) to the subject disperses or disaggregate a stress granule by at least about 10%, at least about 20%, at least about 30%, at least about 40%, at least about 50%, at least about 60%, at least about 70%, at least about 80%, at least about 90%, at least about 95%, or 100% (i.e., complete dispersal) relative to a control (e.g., a baseline of the disaggregation of a stress granule in the subject; the disaggregation of a stress granule in the subject prior to administering a compound disclosed herein; the disaggregation of a stress granule in a subject not receiving a compound disclosed herein; or the disaggregation of a stress granule in a subject receiving a placebo).

In some embodiments, administering a compound disclosed herein (e.g., a compound of Formula (I), (II), (III), or (IV), or subformulas thereof, or a compound of Table 1, or a pharmaceutically acceptable salt thereof) to a subject in need thereof reduces a stress granule level. In some embodiments, administering a compound disclosed herein (e.g., a compound of Formula (I), (II), or (III) or subformulas thereof, or a compound of Table 1) to the subject reduces a stress granule level by at least about 10%, at least about 20%, at least about 30%, at least about 40%, at least about 50%, at least about 60%, at least about 70%, at least about 80%, at least about 90%, at least about 95%, or 100% relative to a control (e.g., a baseline of the stress granule level in the subject; the stress granule level in the subject prior to administering a compound disclosed herein; the stress granule level in a subject not receiving a compound disclosed herein; or the stress granule level in a subject receiving a placebo).

In some embodiments, the stress granule comprises tar DNA binding protein-43 (TDP-43), T-cell intracellular antigen 1 (TIA-1), TIA1 cytotoxic granule-associated RNA binding protein-like 1 (TIAR, TIAL1), GTPase activating protein binding protein 1 (G3BP-1), GTPase activating protein binding protein 2 (G3BP-2), tris tetraprolin (TTP, ZFP36), fused in sarcoma (FUS), or fragile X mental retardation protein (FMRP, FMR1).

In some embodiments, the stress granule comprises tar DNA binding protein-43 (TDP-43), T-cell intracellular antigen 1 (TIA-1), TIA1 cytotoxic granule-associated RNA binding protein-like 1 (TIAR, TIAL1), GTPase activating protein binding protein 1 (G3BP-1), GTPase activating protein binding protein 2 (G3BP-2), fused in sarcoma (FUS), or fragile X mental retardation protein (FMRP, FMR1).

In some embodiments, the stress granule comprises tar DNA binding protein-43 (TDP-43), T-cell intracellular antigen 1 (TIA-1), TIA1 cytotoxic granule-associated RNA binding protein-like 1 (TIAR, TIAL1), GTPase activating protein binding protein 1 (G3BP-1), GTPase activating protein binding protein 2 (G3BP-2), or fused in sarcoma (FUS).

In some embodiments, the stress granule comprises tar DNA binding protein-43 (TDP-43).

In some embodiments, the stress granule comprises T-cell intracellular antigen 1 (TIA-1).

In some embodiments, the stress granule comprises TIA-1 cytotoxic granule-associated RNA binding protein-like 1 (TIAR, TIAL1).

In some embodiments, the stress granule comprises GTPase activating protein binding protein 1 (G3BP-1).

In some embodiments, the stress granule comprises GTPase activating protein binding protein 2 (G3BP-2).

In some embodiments, the stress granule comprises tris tetraprolin (TTP, ZFP36).

In some embodiments, the stress granule comprises fused in sarcoma (FUS).

In some embodiments, the stress granule comprises fragile X mental retardation protein (FMRP, FMR1).

In another aspect, the disclosure provides methods of modulating TDP-43 inclusion formation in a subject, the methods generally comprise administering a compound disclosed herein (e.g., a compound of Formula (I), (II), (III), or (IV), or subformulas thereof, or a compound of Table 1, or a pharmaceutically acceptable salt thereof) to a subject in need thereof. In some embodiments, TDP-43 inclusion formation is inhibited. In some embodiments, the TDP-43 inclusion is disaggregated. In some embodiments, TDP-43 inclusion formation is stimulated.

In some embodiments, administering a compound disclosed herein (e.g., a compound of Formula (I), (II), (III), or (IV), or subformulas thereof, or a compound of Table 1, or a pharmaceutically acceptable salt thereof) to the subject inhibits the formation of a TDP-43 inclusion. In some embodiments, administering a compound disclosed herein (e.g., a compound of Formula (I), (II), (III), or (IV), or subformulas thereof, or a compound of Table 1, or a pharmaceutically acceptable salt thereof) to the subject inhibits the formation of a TDP-43 inclusion by at least about 10%, at least about 20%, at least about 30%, at least about 40%, at least about 50%, at least about 60%, at least about 70%, at least about 80%, at least about 90%, at least about 95%, or 100% (i.e., complete inhibition) relative to a control (e.g., a baseline of the formation of a TDP-43 inclusion in the subject; the formation of a TDP-43 inclusion in the subject prior to administering a compound disclosed herein; the formation of a TDP-43 inclusion in a subject not receiving a compound disclosed herein; or the formation of a TDP-43 inclusion in a subject receiving a placebo).

In some embodiments, administering a compound disclosed herein (e.g., a compound of Formula (I), (II), (III), or (IV), or subformulas thereof, or a compound of Table 1, or a pharmaceutically acceptable salt thereof) to the subject disaggregates a TDP-43 inclusion. In some embodiments, administering a compound disclosed herein (e.g., a compound of Formula (I), (II), (III), or (IV), or subformulas thereof, or a compound of Table 1, or a pharmaceutically acceptable salt thereof) to the subject disperses or disaggregates a TDP-43 inclusion by at least about 10%, at least about 20%, at least about 30%, at least about 40%, at least about 50%, at least about 60%, at least about 70%, at least about 80%, at least about 90%, at least about 95%, or 100% (i.e., complete dispersal) relative to a control (e.g., a baseline of the disaggregation of a TDP-43 in the subject; the disaggregation of a TDP-43 in the subject prior to administering a compound disclosed herein; the disaggregation of a TDP-43 in a subject not receiving a compound disclosed herein; or the disaggregation of a TDP-43 in a subject receiving a placebo).

In some embodiments, administering a compound disclosed herein (e.g., a compound of Formula (I), (II), (III), or (IV), or subformulas thereof, or a compound of Table 1, or a pharmaceutically acceptable salt thereof) modulates the post-translational modification of the TDP-43 protein in a stress granule.

In another aspect, the disclosure provides methods of modulating tau aggregate formation in a subject, the methods generally comprise administering a compound disclosed herein (e.g., a compound of Formula (I), (II), (III), or (IV), or subformulas thereof, or a compound of Table 1, or a pharmaceutically acceptable salt thereof) to a subject in need thereof. In some embodiments, tau aggregate formation is inhibited. In some embodiments, the tau aggregate is disaggregated.

In some embodiments, administering a compound disclosed herein (e.g., a compound of Formula (I), (II), (III), or (IV), or subformulas thereof, or a compound of Table 1, or a pharmaceutically acceptable salt thereof) to the subject inhibits the formation of a tau aggregate. In some embodiments, administering a compound disclosed herein (e.g., a compound of Formula (I), (II), (III), or (IV), or subformulas thereof, or a compound of Table 1, or a pharmaceutically acceptable salt thereof) to the subject inhibits the formation of a tau aggregate by at least about 10%, at least about 20%, at least about 30%, at least about 40%, at least about 50%, at least about 60%, at least about 70%, at least about 80%, at least about 90%, at least about 95%, or 100% (i.e., complete inhibition) relative to a control (e.g., a baseline of the formation of a tau aggregate in the subject; the formation of a tau aggregate in the subject prior to administering a compound disclosed herein; the formation of a tau aggregate in a subject not receiving a compound disclosed herein; or the formation of a tau aggregate in a subject receiving a placebo).

In some embodiments, administering a compound disclosed herein (e.g., a compound of Formula (I), (II), (III), or (IV), or subformulas thereof, or a compound of Table 1, or a pharmaceutically acceptable salt thereof) to the subject disaggregates a tau aggregate. In some embodiments, administering a compound disclosed herein (e.g., a compound of Formula (I), (II), (III), or (IV), or subformulas thereof, or a compound of Table 1, or a pharmaceutically acceptable salt thereof) to the subject disperses or disaggregates a tau aggregate by at least about 10%, at least about 20%, at least about 30%, at least about 40%, at least about 50%, at least about 60%, at least about 70%, at least about 80%, at least about 90%, at least about 95%, or 100% (i.e., complete dispersal) relative to a control (e.g., a baseline of the disaggregation of a tau aggregate in the subject; the disaggregation of a tau aggregate in the subject prior to administering a compound disclosed herein; the disaggregation of a tau aggregate in a subject not receiving a compound disclosed herein; or the disaggregation of a tau aggregate in a subject receiving a placebo).

### Neurodegenerative Diseases

Without being bound by a theory, compounds disclosed herein (e.g., a compound of Formula (I), (II), (III), or (IV), or subformulas thereof, or a compound of Table 1, or a pharmaceutically acceptable salt thereof) can be used to delay the progression of neurodegenerative illnesses where the pathology incorporates stress granules. Such illnesses include ALS and frontotemporal dementia, in which TDP-43 or tau is the predominant protein that accumulates to form the pathology. This group also includes Alzheimer's disease and FTLD-U, where TDP-43 and other stress granule proteins co-localize with tau pathology. Because modulators of TDP-43 inclusions, such as compounds disclosed herein (e.g., a compound of Formula (I), (II), (III), or (IV), or subformulas thereof, or a compound of Table 1, or a pharmaceutically acceptable salt thereof), can act to block the enzymes that signal stress granule formation (e.g., the three enzymes that phosphorylate eIF2a: PERK, GCN2 and HRI), compounds disclosed herein (e.g., a compound of Formula (I), (II), (III), or (IV), or subformulas thereof, or a compound of Table 1, or a pharmaceutically acceptable salt thereof) may also reverse stress granules that might not include TDP-43. Accordingly, compounds disclosed herein (e.g., a compound of Formula (I), (II), (III), or (IV), or subformulas thereof, or a compound of Table 1, or a pharmaceutically acceptable salt thereof) can be used for treatment of neurodegenerative diseases and disorders in which the pathology incorporates stress granules, such as Huntington's chorea and Creutzfeld-Jacob disease. Compounds disclosed herein (e.g., a compound of Formula (I), (II), (III), or (IV), or subformulas thereof, or a compound of Table 1, or a pharmaceutically acceptable salt thereof) may also be used for treatment of neurodegenerative diseases and disorders that involve TDP-43 multisystem proteinopathy.

The term “neurodegenerative disease” as used herein, refers to a neurological disease characterized by loss or degeneration of neurons. The term “neurodegenerative disease” includes diseases caused by the involvement of genetic factors or the cell death (apoptosis) of neurons attributed to abnormal protein accumulation and so on. Additionally, neurodegenerative diseases include neurodegenerative movement disorders and neurodegenerative conditions relating to memory loss or dementia. Neurodegenerative diseases include tauopathies and α-synucleopathies. Exemplary neurodegenerative diseases include, but are not limited to, Alzheimer's disease, frontotemporal dementia (FTD), FTLD-U, FTD caused by mutations in the progranulin protein or tau protein (e.g., progranulin-deficient FTLD), frontotemporal dementia with inclusion body myopathy (IBMPFD), frontotemporal dementia with motor neuron disease, amyotrophic lateral sclerosis (ALS), amyotrophic lateral sclerosis with dementia (ALSD), Huntington's disease (HD), Huntington's chorea, prion diseases (e.g., Creutzfeld-Jacob disease, bovine spongiform encephalopathy, Kuru, or scrapie), Lewy Body disease, diffuse Lewy body disease (DLBD), polyglutamine (polyQ)-repeat diseases, trinucleotide repeat diseases, cerebral degenerative diseases, presenile dementia, senile dementia, Parkinsonism linked to chromosome 17 (FTDP-17), progressive supranuclear palsy (PSP), progressive bulbar palsy (PBP), pseudobulbar palsy, spinal and bulbar muscular atrophy (SBMA), primary lateral sclerosis, Pick's disease, primary progressive aphasia, corticobasal dementia, HIV-associated dementia, Parkinson's disease, Parkinson's disease with dementia, dementia with Lewy bodies, Down's syndrome, multiple system atrophy, spinal muscular atrophy (SMA, e.g., SMA Type I (e.g., Werdnig-Hoffnann disease) SMA Type II, SMA Type III (e.g., Kugelberg-Welander disease), and congenital SMA with arthrogryposis), progressive spinobulbar muscular atrophy (e.g., Kennedy disease), post-polio syndrome (PPS), spinocerebellar ataxia, pantothenate kinase-associated neurodegeneration (PANK), spinal degenerative disease/motor neuron degenerative diseases, upper motor neuron disorder, lower motor neuron disorder, age-related disorders and dementias, Hallervorden-Spatz syndrome, Lytigo-bodig (amyotrophic lateral sclerosis-parkinsonism dementia), Guam-Parkinsonism dementia, hippocampal sclerosis, corticobasal degeneration, Alexander disease, Apler's disease, Krabbe's disease, neuroborreliosis, neurosyphilis, Sandhoff disease, Schilder's disease, Batten disease, Cockayne syndrome, Kearns-Sayre syndrome, Gerstmann-Straussler-Scheinker syndrome, hereditary spastic paraparesis, Leigh's syndrome, demyelinating diseases, epilepsy, tremors, depression, mania, anxiety and anxiety disorders, sleep disorders (e.g., narcolepsy, fatal familial insomnia), acute brain injuries (e.g., stroke, head injury) and autism. As used herein, the term “α-synucleopathy” refers to a neurodegenerative disorder or disease involving aggregation of α-synuclein or abnormal α-synuclein in nerve cells in the brain. A-Synucleopathies include, but are not limited to, Parkinson's disease, Parkinson's disease with dementia, dementia with Lewy bodies, Pick's disease, Down's syndrome, multiple system atrophy, amyotrophic lateral sclerosis (ALS), Hallervorden-Spatz syndrome, and the like.

As used herein, the term “tauopathy” refers to a neurodegenerative disease associated with the pathological aggregation of tau protein in the brain. Tauopathies include, but are not limited to, Alzheimer's disease, Pick's disease, corticobasal degeneration, Argyrophilic grain disease (AGD), progressive supranuclear palsy, Frontotemporal dementia, Frontotemporal lobar degeneration, or Pick's complex.

In some embodiments, the neurodegenerative disease is selected from the group consisting of Alzheimer's disease, frontotemporal dementia (FTD), FTLD-U, FTD caused by mutations in the progranulin protein or tau protein (e.g., progranulin-deficient FTLD), frontotemporal dementia with inclusion body myopathy (IBMPFD), frontotemporal dementia with motor neuron disease, amyotrophic lateral sclerosis (ALS), Huntington's disease (HD), Huntington's chorea, prion diseases (e.g., Creutzfeldt-Jacob disease, bovine spongiform encephalopathy, Kuru, and scrapie), Lewy Body disease, diffuse Lewy body disease (DLBD), polyglutamine (polyQ)-repeat diseases, trinucleotide repeat diseases, cerebral degenerative diseases, presenile dementia, senile dementia, Parkinsonism linked to chromosome 17 (FTDP-17), progressive supranuclear palsy (PSP), progressive bulbar palsy (PBP), pseudobulbar palsy, spinal and bulbar muscular atrophy (SBMA), primary lateral sclerosis, Pick's disease, primary progressive aphasia, corticobasal dementia, HIV-associated dementia, Parkinson's disease, Parkinson's disease with dementia, dementia with Lewy bodies, Down's syndrome, multiple system atrophy, spinal muscular atrophy (SMA, e.g., SMA Type I (e.g., Werdnig-Hoffnann disease), SMA Type II, SMA Type III (e.g., Kugelberg-Welander disease), and congenital SMA with arthrogryposis), progressive spinobulbar muscular atrophy (e.g., Kennedy disease), post-polio syndrome (PPS), spinocerebellar ataxia, pantothenate kinase-associated neurodegeneration (PANK), spinal degenerative disease/motor neuron degenerative diseases, upper motor neuron disorder, lower motor neuron disorder, age-related disorders and dementias, Hallervorden-Spatz syndrome, cerebral infarction, cerebral trauma, chronic traumatic encephalopathy, transient ischemic attack, Lytigo-bodig (amyotrophic lateral sclerosis-parkinsonism dementia), Guam-Parkinsonism dementia, hippocampal sclerosis, corticobasal degeneration, Alexander disease, Apler's disease, Krabbe's disease, neuroborreliosis, neurosyphilis, Sandhoff disease, Tay-Sachs disease, Schilder's disease, Batten disease, Cockayne syndrome, Kearns-Sayre syndrome, Gerstmann-Straussler-Scheinker syndrome and other transmissible spongiform encephalopathies, hereditary spastic paraparesis, Leigh's syndrome, demyelinating diseases, neuronal ceroid lipofuscinoses, epilepsy, tremors, depression, mania, anxiety and anxiety disorders, sleep disorders (e.g., narcolepsy, fatal familial insomnia), acute brain injuries (e.g., stroke, head injury) autism, other diseases or disorders relating to the aberrant expression of or aggregation of TDP-43 or tau and altered proteostasis, and any combination thereof.

In some embodiments, the neurodegenerative disease is selected from the group consisting of Alzheimer's disease, frontotemporal dementia (FTD), FTLD-U, FTD caused by mutations in the progranulin protein or tau protein (e.g., progranulin-deficient FTLD), amyotrophic lateral sclerosis (ALS), Huntington's disease (HD), Huntington's chorea, Creutzfeld-Jacob disease, senile dementia, Parkinsonism linked to chromosome 17 (FTDP-17), progressive supranuclear palsy (PSP), Pick's disease, primary progressive aphasia, corticobasal dementia, Parkinson's disease, Parkinson's disease with dementia, dementia with Lewy bodies, Down's syndrome, multiple system atrophy, spinal muscular atrophy (SMA), spinocerebellar ataxia, spinal degenerative disease/motor neuron degenerative diseases, Hallervorden-Spatz syndrome, cerebral infarction, cerebral trauma, chronic traumatic encephalopathy, transient ischemic attack, Lytigo-bodig (amyotrophic lateral sclerosis-parkinsonism dementia), hippocampal sclerosis, corticobasal degeneration, Alexander disease, Cockayne syndrome, and any combination thereof.

In some embodiments, the neurodegenerative disease is frontotemporal dementia (FTD). In some embodiments, the neurodegenerative disease is Alzheimer's disease or amyotrophic lateral sclerosis (ALS).

### Musculoskeletal Diseases

Musculoskeletal diseases and disorders as defined herein are conditions that affect the muscles, ligaments, tendons, and joints, as well as the skeletal structures that support them. Without being bound by a theory, aberrant expression of certain proteins, such as the full-length isoform of DUX4, has been shown to inhibit protein turnover and increase the expression and aggregation of cytotoxic proteins including insoluble TDP-43 in skeletal muscle cells (Homma, S. et al. Ann Clin Transl Neurol (2015) 2:151-166). As such, compounds of Formula (I), (II), or (III) may be used to prevent or treat a musculoskeletal disease, e.g., a musculoskeletal disease that results in accumulation of TDP-43 and other stress granule proteins, e.g., in the nucleus, cytoplasm, or cell bodies of a muscle cell or motor neuron. Exemplary musculoskeletal diseases include muscular dystrophy, facioscapulohumeral muscular dystrophy (e.g., FSHD1 or FSHD2), Friedrich's ataxia, progressive muscular atrophy (PMA), mitochondrial encephalomyopathy (MELAS), multiple sclerosis, inclusion body myopathy, inclusion body myositis (e.g., sporadic inclusion body myositis), post-polio muscular atrophy (PPMA), motor neuron disease, myotonia, myotonic dystrophy, sarcopenia, spasticity, multifocal motor neuropathy, inflammatory myopathies, paralysis, and other diseases or disorders relating to the aberrant expression of TDP-43 and altered proteostasis. In addition, compounds of Formula (I), (II), or (III) may be used to prevent or treat symptoms caused by or relating to said musculoskeletal diseases, e.g., kyphosis, hypotonia, foot drop, motor dysfunctions, muscle weakness, muscle atrophy, neuron loss, muscle cramps, altered or aberrant gait, dystonias, astrocytosis (e.g., astrocytosis in the spinal cords), liver disease, inflammation, headache, pain (e.g., back pain, neck pain, leg pain, inflammatory pain), and the like. In some embodiments, a musculoskeletal disease or a symptom of a musculoskeletal disease may overlap with a neurodegenerative disease or a symptom of a neurodegenerative disease.

In some embodiments, the musculoskeletal disease is selected from the group consisting of muscular dystrophy, facioscapulohumeral muscular dystrophy (e.g., FSHD1 or FSHD2), Friedrich's ataxia, progressive muscular atrophy (PMA), mitochondrial encephalomyopathy (MELAS), multiple sclerosis, inclusion body myopathy, inclusion body myositis (e.g., sporadic inclusion body myositis), post-polio muscular atrophy (PPMA), motor neuron disease, myotonia, myotonic dystrophy, sarcopenia, multifocal motor neuropathy, inflammatory myopathies, paralysis, and other diseases or disorders relating to the aberrant expression or aggregation of TDP-43 or tau and altered proteostasis.

In some embodiments, a compound disclosed herein (e.g., a compound of Formula (I), (II), (III), or (IV), or subformulas thereof, or a compound of Table 1, or a pharmaceutically acceptable salt thereof) may be used to prevent or treat symptoms caused by or relating to said musculoskeletal diseases, e.g., kyphosis, hypotonia, foot drop, motor dysfunctions, muscle weakness, muscle atrophy, neuron loss, muscle cramps, altered or aberrant gait, dystonias, astrocytosis (e.g., astrocytosis in the spinal cords), liver disease, respiratory disease or respiratory failure, inflammation, headache, and pain (e.g., back pain, neck pain, leg pain, or inflammatory pain).

### Cancers

Cancer cells grow quickly and in low oxygen environments by activating different elements of the cellular stress response. Researchers have shown that drugs targeting different elements of the stress response can be anti-neoplastic. For example, rapamycin blocks mTOR, upregulates autophagy and inhibits some types of tumors. Proteasomal inhibitors, such as velcade (Millennium Pharma) are used to treat some cancers. HSP90 inhibitors, such as 17-allylaminogeldanamycin (17AAG), are currently in clinical trials for cancer. Without being bound by a theory, compounds of Formula (I), (II), or (III) may also be used for treatment of cancer, as a greater understanding of the role of TDP-43 in RNA processing and transcription factor signaling has recently begun to emerge. Additionally, TDP-43 modulators can be combined with one or more cancer therapies, such as chemotherapy and radiation therapy.

A “cancer” in a subject refers to the presence of cells possessing characteristics typical of cancer-causing cells, such as uncontrolled proliferation, immortality, metastatic potential, rapid growth and proliferation rate, and certain characteristic morphological features. Often, cancer cells will be in the form of a tumor, but such cells may exist alone within an animal, or may be a non-tumorigenic cancer cell, such as a leukemia cell. In some circumstances, cancer cells will be in the form of a tumor; such cells may exist locally within an animal, or circulate in the blood stream as independent cells, for example, leukemic cells. Examples of cancer include but are not limited to breast cancer, a melanoma, adrenal gland cancer, biliary tract cancer, bladder cancer, brain or central nervous system cancer, bronchus cancer, blastoma, carcinoma, a chondrosarcoma, cancer of the oral cavity or pharynx, cervical cancer, colon cancer, colorectal cancer, esophageal cancer, gastrointestinal cancer, glioblastoma, hepatic carcinoma, hepatoma, kidney cancer, leukemia, liver cancer, lung cancer, lymphoma, non-small cell lung cancer, ophthalmological cancer, osteosarcoma, ovarian cancer, pancreas cancer, peripheral nervous system cancer, prostate cancer, sarcoma, salivary gland cancer, small bowel or appendix cancer, small-cell lung cancer, squamous cell cancer, stomach cancer, testis cancer, thyroid cancer, urinary bladder cancer, uterine or endometrial cancer, vulval cancer, and the like.

Other exemplary cancers include, but are not limited to, ACTH-producing tumors, acute lymphocytic leukemia, acute nonlymphocytic leukemia, cancer of the adrenal cortex, bladder cancer, brain cancer, breast cancer, cervical cancer, chronic lymphocytic leukemia, chronic myelocytic leukemia, colorectal cancer, cutaneous T-cell lymphoma, endometrial cancer, esophageal cancer, Ewing's sarcoma, gallbladder cancer, hairy cell leukemia, head & neck cancer, ophthalmological cancer, Hodgkin's lymphoma, Kaposi's sarcoma, kidney cancer, liver cancer, lung cancer (small or non-small cell), malignant peritoneal effusion, malignant pleural effusion, melanoma, mesothelioma, multiple myeloma, neuroblastoma, non-Hodgkin's lymphoma, osteosarcoma, ovarian cancer, ovary (germ cell) cancer, prostate cancer, pancreatic cancer, penile cancer, retinoblastoma, skin cancer, soft-tissue sarcoma, squamous cell carcinomas, stomach cancer, testicular cancer, thyroid cancer, trophoblastic neoplasms, uterine cancer, vaginal cancer, cancer of the vulva, Wilm's tumor, and the like.

Exemplary lymphomas include Hodgkin's lymphoma and non-Hodgkin's lymphoma. Further exemplification of non-Hodgkin's lymphoma include, but are not limited to, B-cell lymphomas (e.g., diffuse large B-cell lymphoma, primary mediastinal B-cell lymphoma, intravascular large B-cell lymphoma, follicular lymphoma, chronic lymphocytic leukemia/small lymphocytic lymphoma, mantle cell lymphoma, marginal zone B-cell lymphomas, extranodal marginal B-cell lymphomas, mucosa-associated lymphoid tissue (MALT) lymphomas, modal marginal zone B-cell lymphoma, splenic marginal zone B-cell lymphoma, Burkitt lymphoma, lymphoplasmacytic lymphoma, Waldenström's macroglobulinemia, hairy cell leukemia, and primary central nervous system (CNS) lymphoma) and T-cell lymphomas (e.g., precursor T-lymphoblastic lymphoma/leukemia, peripheral T-cell lymphoma, cutaneous T-cell lymphoma, adult T-cell lymphoma (e.g., smoldering adult T-cell lymphoma, chronic adult T-cell lymphoma, acute adult T-cell lymphoma, lymphomatous adult T-cell lymphoma), angioimmunoblastic T-cell lymphoma, extranodal natural killer T-cell lymphoma nasal type (ENKL), enteropathy-associated intestinal T-cell lymphoma (EATL) (e.g., Type I EATL and Type II EATL), and anaplastic large cell lymphoma (ALCL)).

In some embodiments, the cancer is selected from the group consisting of breast cancer, a melanoma, adrenal gland cancer, biliary tract cancer, bladder cancer, brain or central nervous system cancer, bronchus cancer, blastoma, carcinoma, a chondrosarcoma, cancer of the oral cavity or pharynx, cervical cancer, colon cancer, colorectal cancer, esophageal cancer, gastrointestinal cancer, glioblastoma, hepatic carcinoma, hepatoma, kidney cancer, leukemia, liver cancer, lung cancer, lymphoma, non-small cell lung cancer, ophthalmological cancer, osteosarcoma, ovarian cancer, pancreas cancer, peripheral nervous system cancer, prostate cancer, sarcoma, salivary gland cancer, small bowel or appendix cancer, small-cell lung cancer, squamous cell cancer, stomach cancer, testis cancer, thyroid cancer, urinary bladder cancer, uterine or endometrial cancer, vulval cancer, and any combination thereof.

In some embodiments, the cancer is selected from the group consisting of blastoma, carcinoma, a glioblastoma, hepatic carcinoma, lymphoma, leukemia, and any combination thereof.

In some embodiments, the cancer is selected from Hodgkin's lymphoma or non-Hodgkin's lymphoma. In some embodiments, the cancer is a non-Hodgkin's lymphoma, selected from the group consisting of a B-cell lymphoma (e.g., diffuse large B-cell lymphoma, primary mediastinal B-cell lymphoma, intravascular large B-cell lymphoma, follicular lymphoma, chronic lymphocytic leukemia/small lymphocytic lymphoma, mantle cell lymphoma, marginal zone B-cell lymphomas, extranodal marginal B-cell lymphomas, mucosa-associated lymphoid tissue (MALT) lymphomas, modal marginal zone B-cell lymphoma, splenic marginal zone B-cell lymphoma, Burkitt lymphoma, lymphoplasmacytic lymphoma, Waldenstrom's macroglobulinemia, hairy cell leukemia, and primary central nervous system (CNS) lymphoma) and a T-cell lymphoma (e.g., precursor T-lymphoblastic lymphoma/leukemia, peripheral T-cell lymphoma, cutaneous T-cell lymphoma, adult T-cell lymphoma (e.g., smoldering adult T-cell lymphoma, chronic adult T-cell lymphoma, acute adult T-cell lymphoma, lymphomatous adult T-cell lymphoma), angioimmunoblastic T-cell lymphoma, extranodal natural killer T-cell lymphoma nasal type (ENKL), enteropathy-associated intestinal T-cell lymphoma (EATL) (e.g., Type I EATL and Type II EATL), and anaplastic large cell lymphoma (ALCL)).

### Ophthalmological Diseases

Ophthalmological diseases and disorders (e.g., retinal diseases and disorders) as defined herein affect the retina and other parts of the eye and may contribute to impaired vision and blindness. Several ophthalmological diseases (e.g., retinal diseases) are characterized by the accumulation of protein inclusions and stress granules within or between cells of the eye, e.g., retinal cells and nearby tissues. In addition, an ophthalmological disease (e.g., retinal disease) may also be a symptom of or precursor to neurogenerative diseases, such as ALS and FTD. Therefore, use of compounds that may inhibit formation of protein inclusions and stress granules, including compounds of Formula (I), (II), (III), or (IV), may play an important role in the prevention or treatment of ophthalmological diseases (e.g., retinal diseases).

Exemplary ophthalmological diseases (e.g., retinal diseases) include, but are not limited to, macular degeneration (e.g., age-related macular degeneration), diabetes retinopathy, histoplasmosis, macular hole, macular pucker, Bietti's crystalline dystrophy, retinal detachment, retinal thinning, retinoblastoma, retinopathy of prematurity, Usher's syndrome, vitreous detachment, Refsum disease, retinitis pigmentosa, onchocerciasis, choroideremia, Leber congenital amaurosis, retinoschisis (e.g., juvenile retinoschisis), Stargardt disease, ophthalmoplegia, and the like.

In some embodiments, the ophthalmological disease or disorder (e.g., retinal disease or disorder) is selected from macular degeneration (e.g., age-related macular degeneration), diabetes retinopathy, histoplasmosis, macular hole, macular pucker, Bietti's crystalline dystrophy, retinal detachment, retinal thinning, retinoblastoma, retinopathy of prematurity, Usher's syndrome, vitreous detachment, Refsum disease, retinitis pigmentosa, onchocerciasis, choroideremia, Leber congenital amaurosis, retinoschisis (e.g., juvenile retinoschisis), Stargardt disease, ophthalmoplegia, and the like.

In some embodiments, the ophthalmological disease or disorder (e.g., retinal disease or disorder) is selected from macular degeneration (e.g., age-related macular degeneration), diabetes retinopathy, histoplasmosis, macular hole, macular pucker, Bietti's crystalline dystrophy, retinoblastoma, retinopathy of prematurity, Usher's syndrome, Refsum disease, retinitis pigmentosa, onchocerciasis, choroideremia, Leber congenital amaurosis, retinoschisis (e.g., juvenile retinoschisis), Stargardt disease, and the like.

### Viral Infections

Stress granules often form during viral illnesses, as viral infections often involve hijacking the cellular reproductive machinery toward production of viral proteins. In this case, inhibitors of stress granules can be useful for interfering with viral function. Other viruses appear to inhibit SG formation to prevent the cell from mobilizing a stress response. In such a case, an inducer of stress granules can interfere with viral activity and help combat viral infections (e.g., Salubrinal, an eIF2a phosphatase inhibitor and stress granule inducer). Two viruses for which SG biology has been investigated include West Nile virus and respiratory syncytial virus (RSV). Therefore, use of compounds that may inhibit formation of protein inclusions and stress granules, including compounds of Formula (I), (II), (III), or (IV), may be useful for the prevention or treatment of a viral infection.

Exemplary viruses include, but are not limited to, West Nile virus, respiratory syncytial virus (RSV), Epstein-Barr virus (EBV), hepatitis A, B, C, and D viruses, herpes viruses, influenza viruses, chicken pox, avian flu viruses, smallpox, polio viruses, HIV, Ebola virus, and the like.

In some embodiments, the viral infection is caused by a virus selected from the group consisting of West Nile virus, respiratory syncytial virus (RSV), herpes simplex virus 1, herpes simplex virus 2, Epstein-Barr virus (EBV), hepatitis virus A, hepatitis virus B, hepatitis virus C, influenza viruses, chicken pox, avian flu viruses, smallpox, polio viruses, HIV-1, HIV-2, Ebola virus, and any combination thereof.

In some embodiments, the viral infection is caused by a virus selected from the group consisting of herpes simplex virus 1, herpes simplex virus 2, Epstein-Barr virus (EBV), hepatitis virus A, hepatitis virus B, hepatitis virus C, HIV-1, HIV-2, Ebola virus, and any combination thereof.

In some embodiments, the viral infection is HIV-1 or HIV-2.

### Definitions

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed compositions and methods, because the scope of the disclosure is limited only by the claims. Further, unless otherwise required by context, singular terms shall include pluralities and plural terms shall include the singular.

As used herein, the terms “compounds” and “agent” are used interchangeably to refer to the inhibitors/antagonists/agonists of the compounds disclosed herein. In some embodiments, the compounds are small organic or inorganic molecules, e.g., with molecular weights less than 7500 amu, preferably less than 5000 amu, and even more preferably less than 2000, 1500, 1000, 750, 600, or 500 amu. In some embodiments, one class of small organic or inorganic molecules are non-peptidyl, e.g., containing 2, 1, or no peptide or saccharide linkages.

Unless otherwise indicated, all numbers expressing quantities of ingredients or reaction conditions used herein should be understood as modified in all instances by the term “about.” The term “about” when used in connection with percentages may mean ±1%, ±2%, ±5%, or ±10%.

The singular terms “a,” “an,” and “the” refer to one or to more than one, unless context clearly indicates otherwise. Similarly, the word “or” is intended to include “and” unless the context clearly indicates otherwise.

Although methods and materials similar or equivalent to those described herein can be used in the practice or testing of this disclosure, suitable methods and materials are described below.

As used herein, the term “administer” refers to the placement of a composition into a subject by a method or route which results in at least partial localization of the composition at a desired site such that desired effect is produced. A compound or composition described herein can be administered by any appropriate route known in the art including, but not limited to, oral or parenteral routes, including intravenous, intramuscular, subcutaneous, transdermal, airway (aerosol), pulmonary, nasal, rectal, intrathecal, and topical (including buccal and sublingual) administration.

The terms “decrease”, “reduced”, “reduction”, “decrease” or “inhibit” are all used herein generally to mean a decrease by a statistically significant amount. In some embodiments, the terms “reduced”, “reduction”, “decrease” or “inhibit” mean a decrease by at least 0.1% as compared to a reference level, for example a decrease by at least about 1%, or at least about 5%, or at least about 10%, or at least about 15%, or at least about 20%, or at least about 30%, or at least about 40%, or at least about 50%, or at least about 60%, or at least about 70%, or at least about 80%, or at least about 90% or up to and including a 100% decrease (e.g., absent level as compared to a reference sample), or any decrease between 1-100%, e.g., 10-100% as compared to a reference level.

The terms “increased”, “increase”, “enhance” or “activate” are all used herein to generally mean an increase by a statically significant amount. In some embodiments, the terms “increased”, “increase”, “enhance” or “activate” mean an increase by at least 0.1% as compared to a reference level, for example a decrease by at least about 1%, or at least about 5%, or at least about 10%, or at least about 15%, or at least about 20%, or at least about 30%, or at least about 40%, or at least about 50%, or at least about 60%, or at least about 70%, or at least about 80%, or at least about 90% or up to and including a 100% increase (e.g., absent level as compared to a reference sample), or any increase between 1-100%, e.g., 10-100% as compared to a reference level.

The terms “treat”, “treating”, and “treatment”, as used herein, refer to a method of alleviating, ameliorating, inhibiting, reversing, or slowing down or stopping the progression, aggravation or deterioration of a disease or disorder or its symptoms or associated conditions. In some embodiments, at least one symptom or associated conditions of a disease or disorder is alleviated by at least about 1%, or at least about 5%, or at least about 10%, or at least about 15%, or at least about 20%, or at least about 30%, or at least about 40%, or at least about 50%.

As used herein, a “therapeutically effective amount” or an “effective amount” of a compound or combination refers to an amount of the compound or combination which is effective, upon single or multiple dose administration(s) to a subject, in treating a disease or disorder (e.g., a disorder as described herein) in a subject, or in curing, alleviating, relieving or improving a subject with a disease or disorder (e.g., a disorder as described herein) beyond that expected in the absence of such treatment. Determination of a therapeutically effective amount or an effective amount is well within the capability of those skilled in the art. Generally, a therapeutically effective amount or an effective amount can vary with the subject's history, age, condition, sex, as well as the severity and type of the medical condition in the subject, and administration of other pharmaceutically active agents.

As used herein, a “subject” means a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In some embodiments, the subject is a mammal, e.g., a primate, e.g., a human. The terms, “patient” and “subject” are used interchangeably herein. The terms, “patient” and “subject” are used interchangeably herein.

As used herein, the terms “modulator of stress granule” and “stress granule modulator” refer to compounds and compositions of Formula (I), (II), or (III) that modulate the formation or disaggregation of stress granules.

The term “TDP-43 inclusion” as used herein refers to protein aggregates that comprise TDP-43 proteins. The TDP-43 protein in the inclusion can be wild-type or a mutant form of TDP-43.

As used herein, the terms “modulator of TDP-43 inclusion” and “TDP-43 inclusion modulator” refer to compounds and compositions of Formula (I), (II), or (III) that modulate the formation or disaggregation of cytoplasmic TDP-43 inclusions.

As used herein, the terms “modulator of tau aggregate” and “tau aggregate modulator” refer to compounds and compositions of Formula (I), (II), or (III) that modulate the formation or disaggregation of tau aggregates.

### Selected Chemical Definitions

At various places in the present specification, substituents of compounds disclosed herein are recited in groups or in ranges. It is specifically intended that the compounds disclosed herein include each and every individual subcombination of the members of such groups and ranges. For example, the term “C₁₋₆ alkyl” is specifically intended to individually disclose methyl, ethyl, propyl, butyl, pentyl and hexyl.

For compounds disclosed herein in which a variable appears more than once, each variable can be a different moiety selected from the Markush group defining the variable. For example, where a structure is described having two R groups that are simultaneously present on the same compound; the two R groups can represent different moieties selected from the Markush group defined for R.

It is further appreciated that certain features of compounds disclosed herein, which are, for clarity, described in the context of separate embodiments, can also be provided in combination in a single embodiment. Conversely, various features of the compounds disclosed herein which are, for brevity, described in the context of a single embodiment, can also be provided separately or in any suitable subcombination.

If a compound of the compounds disclosed herein is depicted in the form of a chemical name and as a formula, in case of any discrepancy, the formula shall prevail.

The symbol [symbol] , whether utilized as a bond or displayed perpendicular to a bond indicates the point at which the displayed moiety is attached to the remainder of the structure (e.g., molecule or solid support).

The following terms are intended to have the meanings presented therewith below and are useful in understanding the description and intended scope of the present disclosure.

In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at each position. Combinations of substituents envisioned under this disclosure are preferably those that result in the formation of stable or chemically feasible compounds.

The term “stable”, as used herein, refers to compounds that are not substantially altered when subjected to conditions to allow for their production, detection, recovery, purification, or use for one or more of the purposes disclosed herein.

Suitable substituents for an optionally substituted alkyl, alkenyl, alkylene, alkynyl, heteroalkyl, heteroalkenyl, heteroalkylene, heteroalkynyl, haloalkyl, haloalkenyl, haloalkynyl, haloalkoxy, cycloalkyl, heterocycloalkyl, carbocyclyl, heterocyclyl, aryl, and heteroaryl group include halogen, ═O, —CN, —OR^{cc}, —NR^{dd}R^{ee}, —S(O)^{kk}R^{cc}, —NR^{cc}S(O)₂R^{cc}, —S(O)₂NR^{dd}R^{ee}, —C(═O)OR^{cc}, —OC(═O)OR^{cc}, —OC(═O)R^{cc}, —OC(═S)OR^{cc}, —C(═S)OR^{cc}, —O(C═S) R^{cc}, —C(═O)NR^{dd}R^{ee}, —NR^{cc}C(═O) R^{cc}, —C(═S)NR^{dd}R^{ee}, —NR^{cc}C(═S)R^{cc}, —NR^{cc}(C═O)OR^{cc}, —O(C═O)NR^{dd}R^{ee}, —NR^{cc}(C═S)OR^{cc}, —O(C═S)NR^{dd}R^{ee}, —NR^{cc}(C═O)NR^{dd}R^{ee}, —NR^{cc}(C═S)NR^{dd}R^{ee}, —C(═S)R^{cc}, —C(═O)R^{cc}, C₁-C₆ alkyl, C₂-C₆ alkenyl, C₂-C₆ alkynyl, C₂-C₉ heteroalkynyl, C₁-C₆ haloalkyl, C₁-C₆ haloalkoxy, C₁-C₆ heteroalkyl, carbocyclyl, (C₁-C₆-alkylene)-carbocyclyl, (C₁-C₆-heteroalkylene)-carbocyclyl, heterocyclyl, (C₁-C₆-alkylene)-heterocyclyl, (C₁-C₆-heteroalkylene)-heterocyclyl, aryl, (C₁-C₆-alkylene)-aryl, (C₁-C₆-heteroalkylene)-aryl, heteroaryl, (C₁-C₆-alkylene)-heteroaryl, or (C₁-C₆-heteroalkylene)-heteroaryl, wherein each of said alkyl, alkenyl, alkylene, alkynyl, heteroalkyl, heteroalkenyl, heteroalkylene, heteroalkynyl, haloalkyl, haloalkenyl, haloalkynyl, haloalkoxy, cycloalkyl, heterocycloalkyl, carbocyclyl, heterocyclyl, aryl and heteroaryl are optionally substituted with one or more of halogen, OR^{cc}, —NO₂, —CN, —NR^{cc}C(═O)W, —NR^{dd}R^{ee}, —S(O)ₖR^{cc}, —C(═O)OR, —C(═O)NR^{dd}R^{ee}, —C(═O)R^{cc}, C₁-C₆ alkyl, C₁-C₆ haloalkyl, C₁-C₆ heteroalkyl, cycloalkyl, heterocycloalkyl, carbocyclyl, heterocyclyl, aryl, or heteroaryl, and wherein R^{cc} is hydrogen, hydroxy, C₁-C₆ alkyl, C₁-C₆ heteroalkyl, carbocyclyl, (C₁-C₆-alkylene)-carbocyclyl, (C₁-C₆-heteroalkylene)-carbocyclyl, heterocyclyl, (C₁-C₆-alkylene)-heterocyclyl, (C₁-C₆-heteroalkylene)-heterocyclyl, aryl, (C₁-C₆-alkylene)-aryl, (C₁-C₆-heteroalkylene)-aryl, heteroaryl, (C₁-C₆-alkylene)-heteroaryl, or (C₁-C₆-heteroalkylene)-heteroaryl, each of which may be optionally substituted with one or more of halogen, hydroxy, C₁-C₆ alkyl, C₁-C₆ haloalkyl, C₁-C₆ heteroalkyl, carbocyclyl, heterocyclyl, aryl, or heteroaryl; R^{dd} and R^{ee} are each independently selected from hydrogen, C₁-C₆ alkyl, or C₁-C₆ heteroalkyl; and k is 0, 1 or 2. The compounds disclosed herein are not intended to be limited in any manner by the above exemplary listing of substituents.

As used herein, the term “optional substituent” refers to any of the suitable substituents for an alkyl, alkenyl, alkylene, alkynyl, heteroalkyl, heteroalkenyl, heteroalkylene, heteroalkynyl, haloalkyl, haloalkenyl, haloalkynyl, haloalkoxy, cycloalkyl, heterocycloalkyl, carbocyclyl, heterocyclyl, aryl, and heteroaryl group, including: halogen, ═O, —CN, —OR^{cc}, —NR^{dd}R^{ee}, —S(O)^{kk}R^{cc}, —NR^{cc}S(O)₂R^{cc}, —S(O)₂NR^{dd}R^{ee}, —C(═O)OR^{cc}, —OC(═O)OR^{cc}, —OC(═O)R^{cc}, —OC(═S)OR^{cc}, —C(═S)OR^{cc}, —O(C═S) R^{cc}, —C(═O)NR^{dd}R^{ee}, —NR^{cc}C(═O) R^{cc}, —C(═S)NR^{dd}R^{ee}, —NR^{cc}C(═S)R^{cc}, —NR^{cc}(C═O)OR^{cc}, —O(C═O)NR^{dd}R^{ee}, —NR^{cc}(C═S)OR^{cc}, —O(C═S)NR^{dd}R^{ee}, —NR^{cc}(C═O)NR^{dd}R^{ee}, —NR^{cc}(C═S)NR^{dd}R^{ee}, —C(═S)R^{cc}, —C(═O)R^{cc}, C₁-C₆ alkyl, C₂-C₆ alkenyl, C₂-C₆ alkynyl, C₂-C₉ heteroalkynyl, C₁-C₆ haloalkyl, C₁-C₆ haloalkoxy, C₁-C₆ heteroalkyl, carbocyclyl, (C₁-C₆-alkylene)-carbocyclyl, (C₁-C₆-heteroalkylene)-carbocyclyl, heterocyclyl, (C₁-C₆-alkylene)-heterocyclyl, (C₁-C₆-heteroalkylene)-heterocyclyl, aryl, (C₁-C₆-alkylene)-aryl, (C₁-C₆-heteroalkylene)-aryl, heteroaryl, (C₁-C₆-alkylene)-heteroaryl, or (C₁-C₆-heteroalkylene)-heteroaryl, wherein each of said alkyl, alkenyl, alkylene, alkynyl, heteroalkyl, heteroalkenyl, heteroalkylene, heteroalkynyl, haloalkyl, haloalkenyl, haloalkynyl, haloalkoxy, cycloalkyl, heterocycloalkyl, carbocyclyl, heterocyclyl, aryl and heteroaryl are optionally substituted with one or more of halogen, —OR^{cc}, —NO₂, —CN, —NR^{cc}C(═O)R^{dd}, —NR^{dd}R^{ee}, —S(O)ₖR^{cc}, —C(═O)OR^{cc}, —C(═O)NR^{dd}R^{ee}, —C(═O)R^{cc}, C₁-C₆ alkyl, C₁-C₆ haloalkyl, C₁-C₆ heteroalkyl, cycloalkyl, heterocycloalkyl, carbocyclyl, heterocyclyl, aryl, or heteroaryl, and wherein R^{cc} is hydrogen, hydroxy, C₁-C₆ alkyl, C₁-C₆ heteroalkyl, carbocyclyl, (C₁-C₆-alkylene)-carbocyclyl, (C₁-C₆-heteroalkylene)-carbocyclyl, heterocyclyl, (C₁-C₆-alkylene)-heterocyclyl, (C₁-C₆-heteroalkylene)-heterocyclyl, aryl, (C₁-C₆-alkylene)-aryl, (C₁-C₆-heteroalkylene)-aryl, heteroaryl, (C₁-C₆-alkylene)-heteroaryl, or (C₁-C₆-heteroalkylene)-heteroaryl, each of which may be optionally substituted with one or more of halogen, hydroxy, C₁-C₆ alkyl, C₁-C₆ haloalkyl, C₁-C₆ heteroalkyl, carbocyclyl, heterocyclyl, aryl, or heteroaryl; R^{dd} and R^{ee} are each independently selected from hydrogen, C₁-C₆ alkyl, or C₁-C₆ heteroalkyl; and k is 0, 1 or 2. The compounds disclosed herein are not intended to be limited in any manner by the above exemplary listing of substituents.

As used herein, “alkyl” refers to a radical of a straight-chain or branched, saturated hydrocarbon group having from 1 to 24 carbon atoms (“C₁-C₂₄ alkyl”). In some embodiments, an alkyl group has 1 to 12 carbon atoms (“C₁-C₁₂ alkyl”). In some embodiments, an alkyl group has 1 to 8 carbon atoms (“C₁-C₈ alkyl”). In some embodiments, an alkyl group has 1 to 6 carbon atoms (“C₁-C₆ alkyl”). In some embodiments, an alkyl group has 1 to 5 carbon atoms (“C₁-C₅ alkyl”). In some embodiments, an alkyl group has 1 to 4 carbon atoms (“C₁-C₄ alkyl”). In some embodiments, an alkyl group has 1 to 3 carbon atoms (“C₁-C₃ alkyl”). In some embodiments, an alkyl group has 1 to 2 carbon atoms (“C₁-C₂ alkyl”). In some embodiments, an alkyl group has 1 carbon atom (“C₁ alkyl”). In some embodiments, an alkyl group has 2 to 6 carbon atoms (“C₂-C₆ alkyl”). Examples of C₁-C₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Each instance of an alkyl group may be independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In some embodiments, the alkyl group is unsubstituted C₁₋₁₀ alkyl (e.g., —CH₃). In some embodiments, the alkyl group is substituted C₁₋₆ alkyl. As used herein, the term “alkylene” refers to an alkyl group with one additional open valence, i.e., a bivalent group. Exemplary alkylene groups include, but are not limited to —CH₂CH₂— and —CH₂—C(CH₃)—CH₂—.

As used herein, “alkenyl” refers to a radical of a straight-chain or branched hydrocarbon group having from 2 to 24 carbon atoms, one or more carbon-carbon double bonds, and no triple bonds (“C₂-C₂₄ alkenyl”). In some embodiments, an alkenyl group has 2 to 10 carbon atoms (“C₂-C₁₀ alkenyl”). In some embodiments, an alkenyl group has 2 to 8 carbon atoms (“C₂-C₈ alkenyl”). In some embodiments, an alkenyl group has 2 to 6 carbon atoms (“C₂-C₆ alkenyl”). In some embodiments, an alkenyl group has 2 to 5 carbon atoms (“C₂-C₅ alkenyl”). In some embodiments, an alkenyl group has 2 to 4 carbon atoms (“C₂-C₄ alkenyl”). In some embodiments, an alkenyl group has 2 to 3 carbon atoms (“C₂-C₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂-C₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂-C₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Each instance of an alkenyl group may be independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In some embodiments, the alkenyl group is unsubstituted C₂₋₁₀ alkenyl. In some embodiments, the alkenyl group is substituted C₂₋₆ alkenyl.

As used herein, the term “alkynyl” refers to a radical of a straight-chain or branched hydrocarbon group having from 2 to 24 carbon atoms, one or more carbon-carbon triple bonds (“C₂-C₂₄ alkenyl”). In some embodiments, an alkynyl group has 2 to 10 carbon atoms (“C₂-C₁₀ alkynyl”). In some embodiments, an alkynyl group has 2 to 8 carbon atoms (“C₂-C₈ alkynyl”). In some embodiments, an alkynyl group has 2 to 6 carbon atoms (“C₂-C₆ alkynyl”). In some embodiments, an alkynyl group has 2 to 5 carbon atoms (“C₂-C₅ alkynyl”). In some embodiments, an alkynyl group has 2 to 4 carbon atoms (“C₂-C₄ alkynyl”). In some embodiments, an alkynyl group has 2 to 3 carbon atoms (“C₂-C₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂-C₄ alkynyl groups include ethynyl (C₂), 1-propynyl (C₃), 2-propynyl (C₃), 1-butynyl (C₄), 2-butynyl (C₄), and the like. Each instance of an alkynyl group may be independently optionally substituted, i.e., unsubstituted (an “unsubstituted alkynyl”) or substituted (a “substituted alkynyl”) with one or more substituents e.g., for instance from 1 to 5 substituents, 1 to 3 substituents, or 1 substituent. In some embodiments, the alkynyl group is unsubstituted C₂₋₁₀ alkynyl. In some embodiments, the alkynyl group is substituted C₂₋₆ alkynyl.

As used herein, the term “heteroalkyl,” refers to a non-cyclic stable straight-chain or branched, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) 0, N, P, S, and Si may be placed at any position of the heteroalkyl group. Exemplary heteroalkyl groups include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —NHCH₂—, —C(O)NH—, —C(O)N(CH₃), —C(O)N(CH₂CH₃)—, —C(O)N(CH₂CF₃)—, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, and —O—CH₂—CH₃. Up to two or three heteroatoms may be consecutive, such as, for example, —CH₂—NH—OCH₃ and —CH₂—O—Si(CH₃)₃. Where “heteroalkyl” is recited, followed by recitations of specific heteroalkyl groups, such as —CH₂O, —NR^{C}R^{D}, or the like, it will be understood that the terms heteroalkyl and —CH₂O or —NR^{C}R^{D} are not redundant or mutually exclusive. Rather, the specific heteroalkyl groups are recited to add clarity. Thus, the term “heteroalkyl” should not be interpreted herein as excluding specific heteroalkyl groups, such as —CH₂O, —NR^{C}R^{D}, or the like.

As used herein, the term “heteroalkylene” refers to a heteroalkyl group with one additional open valence, i.e., a bivalent group. Exemplary heteroalkylene groups include, but are not limited to: —CH₂—CH₂—O—CH₂—, —CH₂—O—, and —CH₂—CH₂—NH—CH₂—.

As used herein, “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆-C₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). An aryl group may be described as, e.g., a C₆-C₁₀-membered aryl, wherein the term “membered” refers to the non-hydrogen ring atoms within the moiety. Each instance of an aryl group may be independently optionally substituted, i.e., unsubstituted (e.g., “unsubstituted aryl”) or substituted (e.g., “substituted aryl”) with one or more substituents. In some embodiments, the aryl group is unsubstituted C₆-C₁₄ aryl. In some embodiments, the aryl group is substituted C₆-C₁₄ aryl. Exemplary aryl groups include, but are not limited to, phenyl, naphthyl, and anthracyl.

As used herein, “heteroaryl” refers to a radical of a 5-14 membered monocyclic or polycyclic (e.g., bicyclic or tricyclic) 4n+2 aromatic ring system (e.g., having 6, 10, or 14 π electrons shared in a cyclic array) having ring carbon atoms and one or more (e.g., 1-4) ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). A heteroaryl group may be described as, e.g., a 5-10-membered heteroaryl, wherein the term “membered” refers to the non-hydrogen ring atoms within the moiety. In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Each instance of a heteroaryl group may be independently optionally substituted, i.e., unsubstituted (an “unsubstituted heteroaryl”) or substituted (a “substituted heteroaryl”) with one or more substituents. In some embodiments, the heteroaryl group is unsubstituted 5-14 membered heteroaryl. In some embodiments, the heteroaryl group is substituted 5-14 membered heteroaryl. Exemplary heteroaryl groups include, but are not limited to, imidazolyl, pyridinyl, and quinolinyl.

As used herein, “cycloalkyl” refers to a radical of a monocyclic or polycyclic (e.g., bicyclic or tricyclic) ring system that is saturated or partially unsaturated, but not aromatic, and has from 3 to 14 ring carbon atoms (“C₃-C₁₄ carbocyclyl”) and zero heteroatoms in the saturated or partially unsaturated, but not aromatic, ring system. In some embodiments, a cycloalkyl group has 3 to 8 ring carbon atoms (“C₃-C₈ cycloalkyl”). In some embodiments, a cycloalkyl group has 3 to 6 ring carbon atoms (“C₃-C₆ cycloalkyl”). In some embodiments, a cycloalkyl group has 3 to 6 ring carbon atoms (“C₃-C₆ cycloalkyl”). In some embodiments, a cycloalkyl group has 5 to 10 ring carbon atoms (“C₅-C₁₀ cycloalkyl”). A cycloalkyl group may be described as, e.g., a C₄-C₇-membered cycloalkyl, wherein the term “membered” refers to the non-hydrogen ring atoms within the moiety. Exemplary C₃-C₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₈ cycloalkyl groups include, without limitation, the aforementioned C₃-C₆ cycloalkyl groups as well as cycloheptyl (C₇), cycloheptenyl (C₇), cycloheptadienyl (C₇), cycloheptatrienyl (C₇), cyclooctyl (C₈), cyclooctenyl (C₈), cubanyl (C₈), bicyclo[1.1.1]pentanyl (C₅), bicyclo[2.2.2]octanyl (C₈), bicyclo[2.1.1]hexanyl (C₆), bicyclo[3.1.1]heptanyl (C₇), and the like. Exemplary C₃-C₁₀ cycloalkyl groups include, without limitation, the aforementioned C₃-C₈ cycloalkyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, the cycloalkyl group is monocyclic (“monocyclic cycloalkyl”) or contains a fused, bridged or spiro ring system such as a bicyclic system (“bicyclic cycloalkyl”) and can be saturated or can be partially unsaturated. Each instance of a cycloalkyl group may be independently optionally substituted, i.e., unsubstituted (an “unsubstituted cycloalkyl”) or substituted (a “substituted cycloalkyl”) with one or more substituents. In some embodiments, the cycloalkyl group is unsubstituted C₃-C₁₀ cycloalkyl. In some embodiments, the cycloalkyl group is a substituted C₃-C₁₀ cycloalkyl. Exemplary cycloalkyl groups include, but are not limited to, cyclohexanyl, cyclohexenyl, cyclooctynyl, and bicyclo[4.4.0]decanyl.

“Heterocycloalkyl” as used herein refers to a radical of a monocyclic or polycyclic (e.g., bicyclic or tricyclic) ring system that is saturated or partially unsaturated, but not aromatic, and has from 3 to 14 ring atoms including carbon and 1 to 6 ring heteroatoms, wherein each heteroatom is independently selected from nitrogen, oxygen, sulfur (e.g., —S—, —S(O)—, and —S(O)₂—), boron, phosphorus, and silicon (“3-14 membered heterocycloalkyl”). In heterocycloalkyl groups that contain one or more nitrogen atoms, the point of attachment can be a carbon or nitrogen atom, as valency permits. A heterocycloalkyl group can either be monocyclic (“monocyclic heterocycloalkyl”) or polycyclic (“polycyclic heterocycloalkyl”), such as bicyclic (“bicyclic heterocycloalkyl”). A heterocycloalkyl group can be a fused, bridged or spiro ring system. Bicyclic heterocycloalkyl can include one or more heteroatoms in one or both rings. A heterocycloalkyl group may be described as, e.g., a 3-7-membered heterocycloalkyl, wherein the term “membered” refers to the non-hydrogen ring atoms, i.e., carbon, nitrogen, oxygen, sulfur, boron, phosphorus, and silicon, within the moiety. Each instance of heterocycloalkyl may be independently optionally substituted, i.e., unsubstituted (an “unsubstituted heterocycloalkyl”) or substituted (a “substituted heterocycloalkyl”) with one or more substituents. In some embodiments, the heterocycloalkyl group is unsubstituted 3-14 membered heterocycloalkyl. In some embodiments, the heterocycloalkyl group is substituted 3-14 membered heterocycloalkyl. In some embodiments, a heterocycloalkyl group is a 5-10 membered heterocycloalkyl having ring carbon atoms and 1-4 ring heteroatoms, wherein each heteroatom is independently selected from nitrogen, oxygen, sulfur, boron, phosphorus, and silicon (“5-10 membered heterocycloalkyl”). In some embodiments, a heterocycloalkyl group is a 5-8 membered heterocycloalkyl having ring carbon atoms and 1-4 ring heteroatoms, wherein each heteroatom is independently selected from nitrogen, oxygen, and sulfur (“5-8 membered heterocycloalkyl”). In some embodiments, a heterocycloalkyl group is a 5-6 membered heterocycloalkyl having ring carbon atoms and 1-4 ring heteroatoms, wherein each heteroatom is independently selected from nitrogen, oxygen, and sulfur (“5-6 membered heterocycloalkyl”). In some embodiments, the 5-6 membered heterocycloalkyl has 1-3 ring heteroatoms, wherein each heteroatom is independently selected from nitrogen, oxygen, and sulfur. In some embodiments, the 5-6 membered heterocycloalkyl has 1-2 ring heteroatoms, wherein each heteroatom is independently selected from nitrogen, oxygen, and sulfur. In some embodiments, the 5-6 membered heterocycloalkyl has one ring heteroatom selected from nitrogen, oxygen, and sulfur. Exemplary heterocycloalkyl groups include, but are not limited to, piperidinyl, piperazinyl, 3,4-dihydro-2H-pyranyl, and octahydroindolyl.

As used herein, “carbocyclyl” refers to a radical of a polycyclic, partially unsaturated ring system having from 6 to 20 carbon atoms and at least one fused aryl ring. The term “membered” refers to the non-hydrogen ring atoms within the moiety. Each instance of a carbocyclyl group may be independently optionally substituted, i.e., unsubstituted (an “unsubstituted carbocyclyl”) or substituted (a “substituted carbocyclyl”) with one or more substituents. In some embodiments, the carbocyclyl group is unsubstituted 6-20 membered carbocyclyl. In some embodiments, the carbocyclyl group is a substituted 6-20 membered carbocyclyl. Exemplary carbocyclyl groups include, but are not limited to, indenyl and tetrahydronaphthyl.

As used herein, “heterocyclyl” refers to a radical of a polycyclic, partially unsaturated ring system having from 5 to 20 atoms (“5-20 membered heterocyclyl”) including carbon and 1 to 6 heteroatoms, wherein each heteroatom is independently selected from nitrogen, oxygen, sulfur, boron, phosphorus, and silicon, and wherein the polycyclic, partially unsaturated ring system has at least one aromatic ring (e.g., aryl or heteroaryl). In some embodiments, a heterocyclyl group has 5 to 14 ring atoms (“5-14 membered heterocyclyl”). The term “membered” refers to the non-hydrogen ring atoms within the moiety. Each instance of a heterocyclyl group may be independently optionally substituted, i.e., unsubstituted (an “unsubstituted heterocyclyl”) or substituted (a “substituted heterocyclyl”) with one or more substituents. In some embodiments, the heterocyclyl group is unsubstituted 5-20 membered heterocyclyl. In some embodiments, the heterocyclyl group is a substituted 5-20 membered heterocyclyl. Exemplary heterocyclyl groups include, but are not limited to, 1,2,3,4-tetrahydroquinolyl, 7,8-dihydro-5H-pyrano[4,3-b]pyridinyl, 1,4,6,7-tetrahydropyrano[4,3-b]pyrrole, 6,7-dihydro-5H-cyclopenta[b]pyridinyl, and 5,6,7,8-tetrahydroquinolinyl.

As used herein, “cyano” refers to the radical —CN.

As used herein, “halo” or “halogen,” independently or as part of another substituent, mean, unless otherwise stated, a fluorine (F), chlorine (Cl), bromine (Br), or iodine (I) atom.

As used herein, “haloalkyl” can include alkyl structures that are substituted with one or more halo groups or with combinations thereof. For example, the terms “fluoroalkyl” includes haloalkyl groups in which the halo is fluorine (e.g., —C₁-C₆ alkyl-CF₃, —C₁-C₆ alkyl-C₂F). Non-limiting examples of haloalkyl include trifluoroethyl, trifluoropropyl, trifluoromethyl, fluoromethyl, difluoromethyl, and fluoroisopropyl.

As used herein, “hydroxy” refers to the radical —OH.

As used herein, “nitro” refers to —NO₂.

As used herein, “oxo” (═O) refers to a carbonyl, in which both bonds from the oxygen are connected to the same atom. For example, a carbon atom substituted with oxo forms a carbonyl group C═O.

Two or more substituents may optionally be joined to form aryl, heteroaryl, cycloalkyl, heterocycloalkyl, carbocyclyl, or heterocyclyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Compounds described herein can comprise one or more asymmetric centers, and thus can exist in various isomeric forms, e.g., enantiomers or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC); or preferred isomers can be prepared by asymmetric syntheses. The disclosure additionally encompasses compounds described herein as individual isomers substantially free of other isomers, and alternatively, as mixtures of various isomers.

As used herein, a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 850% by weight, more than 90% by weight, more than 910% by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the enantiomer. In some embodiments, the weights are based upon total weight of all enantiomers or stereoisomers of the compound.

In the compositions provided herein, an enantiomerically pure compound can be present with other active or inactive ingredients. For example, a pharmaceutical composition comprising enantiomerically pure R-compound can comprise, for example, about 90% excipient and about 10% enantiomerically pure R-compound. In some embodiments, the enantiomerically pure R-compound in such compositions can, for example, comprise, at least about 95% by weight R-compound and at most about 5% by weight S-compound, by total weight of the compound. For example, a pharmaceutical composition comprising enantiomerically pure S-compound can comprise, for example, about 90% excipient and about 10% enantiomerically pure S-compound. In some embodiments, the enantiomerically pure S-compound in such compositions can, for example, comprise, at least about 95% by weight S-compound and at most about 5% by weight R-compound, by total weight of the compound. In some embodiments, the active ingredient can be formulated with little or no excipient or carrier.

Compound disclosed herein may also comprise one or more isotopic substitutions. Isotopes include those atoms having the same atomic number but different mass numbers. For example, H may be in any isotopic form, including ¹H, ²H (D or deuterium), and ³H (T or tritium); C may be in any isotopic form, including ¹²C, ¹³C, and ¹⁴C; O may be in any isotopic form, including ¹⁶O and ¹⁸O; and the like. Unless otherwise stated, when a position is designated specifically as “H” or “hydrogen,” the position is understood to have hydrogen at its natural abundance isotopic composition. Further, unless otherwise stated, when a position is designated specifically as “D” or “deuterium,” the position is understood to have deuterium at an abundance that is at least 3000 times greater than the natural abundance of deuterium, which is 0.015% (i.e., the term “D” or “deuterium” indicates at least about 45% incorporation of deuterium).

One or more constituent atoms of the compounds of the present disclosure can be replaced or substituted with isotopes of the atoms in non-natural abundance. In some embodiments, the compound comprises one or more deuterium atoms. For example, one or more hydrogen atoms in a compound disclosed herein can be replaced or substituted by deuterium. In some embodiments, the compound comprises two or more deuterium atoms. In some embodiments, the compound comprises 1, 2, 3, 4, 5, 6, 7, 8, or 9 deuterium atoms. Synthetic methods for including isotopes into organic compounds are known in the art.

Many of the terms given above may be used repeatedly in the definition of a formula or group and in each case have one of the meanings given above, independently of one another.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

Contemplated equivalents of the compounds described above include compounds which otherwise correspond thereto, and which have the same general properties thereof (e.g., the ability to inhibit the formation of TDP-43 inclusions), wherein one or more simple variations of substituents are made which do not adversely affect the efficacy of the compound. In general, the compounds of the present disclosure may be prepared by the methods illustrated in the general reaction schemes as, for example, described below, or by modifications thereof, using readily available starting materials, reagents and conventional synthesis procedures. In these reactions, it is also possible to make use of variants which are in themselves known, but are not mentioned here.

For purposes of this disclosure, the chemical elements are identified in accordance with the Periodic Table of the Elements, CAS version, Handbook of Chemistry and Physics, 67th Ed., 1986-87, inside cover. As used herein, the term “hydrocarbon” is contemplated to include all permissible compounds having at least one hydrogen and one carbon atom. In a broad aspect, the permissible hydrocarbons include acyclic and cyclic, branched and unbranched, carbocyclic and heterocyclic, aromatic and nonaromatic organic compounds which can be substituted or unsubstituted.

### Pharmaceutical Compositions and Routes of Administration

Pharmaceutical compositions containing compounds disclosed herein (e.g., a compound of Formula (I), (II), (III), or (IV), or subformulas thereof, or a compound of Table 1, or a pharmaceutically acceptable salt thereof) can be used to treat or ameliorate a disorder described herein, for example, a neurodegenerative disease, a cancer, an ophthalmological disease (e.g., a retinal disease), or a viral infection.

The amount and concentration of compounds disclosed herein (e.g., a compound of Formula (I), (II), (III), or (IV), or subformulas thereof, or a compound of Table 1, or a pharmaceutically acceptable salt thereof) in the pharmaceutical compositions, as well as the quantity of the pharmaceutical composition administered to a subject, can be selected based on clinically relevant factors, such as medically relevant characteristics of the subject (e.g., age, weight, gender, other medical conditions, and the like), the solubility of compounds in the pharmaceutical compositions, the potency and activity of the compounds, and the manner of administration of the pharmaceutical compositions.

While it is possible for a compound disclosed herein (e.g., a compound of Formula (I), (II), (III), or (IV), or subformulas thereof, or a compound of Table 1, or a pharmaceutically acceptable salt thereof) to be administered alone, it is preferable to administer the compound as a pharmaceutical composition, where the compound is combined with one or more pharmaceutically acceptable diluents, excipients or carriers.

Compounds disclosed herein (e.g., a compound of Formula (I), (II), (III), or (IV), or subformulas thereof, or a compound of Table 1, or a pharmaceutically acceptable salt thereof) may be formulated for administration in any convenient way for use in human or veterinary medicine. In some embodiments, the compound included in the pharmaceutical preparation may be active itself, or may be a prodrug, e.g., capable of being converted to an active compound in a physiological setting. Regardless of the route of administration selected, the compounds of the present disclosure or the pharmaceutical compositions of the present disclosure are formulated into pharmaceutically acceptable dosage forms such as described below or by other conventional methods known to those of skill in the art.

Thus, another aspect of the present disclosure provides pharmaceutically acceptable compositions comprising an effective amount of one or more of the compounds disclosed herein (e.g., a compound of Formula (I), (II), (III), or (IV), or subformulas thereof, or a compound of Table 1, or a pharmaceutically acceptable salt thereof), formulated together with one or more pharmaceutically acceptable carriers, additives, or diluents. The pharmaceutical compositions disclosed herein may be specially formulated for administration in solid or liquid form. The pharmaceutical compositions disclosed herein may be administered to a subject by various routes of administration including, but not limited to: oral administration; parenteral administration; topical application; intravaginally or intrarectally, sublingually; ocularly; transdermally; transmucosally; nasally; or intrathecally. Additionally, compounds disclosed herein can be implanted into a patient or injected using a drug delivery system.

The phrase “therapeutically effective amount” as used herein means that amount of a compound, material, or composition comprising a compound disclosed herein (e.g., a compound of Formula (I), (II), (III), or (IV), or subformulas thereof, or a compound of Table 1, or a pharmaceutically acceptable salt thereof) which is effective for producing some desired therapeutic effect (e.g., by inhibiting TDP-43 inclusions, in at least a sub-population of cells in an animal and thereby blocking the biological consequences of that function in the treated cells, at a reasonable benefit/risk ratio applicable to any medical treatment).

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

The phrase “pharmaceutically acceptable” is employed herein to refer to those compounds, materials, compositions, or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject antagonists from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include, but are not limited to: sugars; starches; cellulose; powdered tragacanth; malt; gelatin; talc; excipients; oils; glycols; polyols; esters; agar; buffering agents; alginic acid; pyrogen-free water; isotonic saline; Ringer's solution; ethyl alcohol; phosphate buffer solutions; cyclodextrins; and other nontoxic compatible substances employed in pharmaceutical formulations.

The term “pharmaceutically acceptable salt” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain compounds disclosed herein contain both basic and acidic functionalities that allow the compounds to be converted into either base or acid addition salts. These salts may be prepared by methods known to those skilled in the art. Other pharmaceutically acceptable carriers known to those of skill in the art are suitable for the present disclosure.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the present disclosure include those suitable for oral, nasal, topical (including buccal and sublingual), rectal, vaginal or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect.

Methods of preparing these formulations or compositions include the step of bringing into association a compound of the present disclosure with the carrier and, optionally, one or more accessory ingredients. In general, the formulations are prepared by uniformly and intimately bringing into association a compound of the present disclosure with liquid carriers, or finely divided solid carriers, or both, and then, if necessary, shaping the product.

Formulations of the invention suitable for oral administration may be in the form of capsules, cachets, pills, tablets, lozenges, powders, granules, or as a solution or a suspension. In solid dosage forms of the disclosure for oral administration, the active ingredient is mixed with one or more pharmaceutically acceptable carriers, including, but not limited to: fillers or extenders; binders; humectants; disintegrating agents; solution retarding agents; absorption accelerators; wetting agents; absorbents; lubricants; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebrospinal, and intrasternal injection and infusion. In some embodiments, the compositions are administered by intravenous infusion or injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Examples of suitable aqueous and nonaqueous carriers that may be employed in the pharmaceutical compositions disclosed herein include water, ethanol, polyols, and suitable mixtures thereof, vegetable oils, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

### Dosages

Actual dosage levels of the active ingredients in the pharmaceutical compositions disclosed herein may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound of the present disclosure employed, or the ester, salt or amide thereof, the route of administration, the time of administration, the rate of excretion of the particular compound being employed, the duration of the treatment, other drugs, compounds or materials used in combination with the particular compound employed, the age, sex, weight, condition, general health and prior medical history of the patient being treated, and like factors well known in the medical arts.

Toxicity and therapeutic efficacy can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferred.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized.

The therapeutically effective dose can be estimated initially from cell culture assays. A dose may be formulated in animal models to achieve a circulating plasma concentration range that includes the IC₅₀ (i.e., the concentration of the therapeutic which achieves a half-maximal inhibition of symptoms) as determined in cell culture. Levels in plasma may be measured, for example, by high performance liquid chromatography. The effects of any particular dosage can be monitored by a suitable bioassay.

The dosage may be determined by a physician and adjusted, as necessary, to suit observed effects of the treatment. Generally, the compositions are administered so that a compound disclosed herein (e.g., a compound of Formula (I), (II), (III), or (IV), or subformulas thereof, or a compound of Table 1, or a pharmaceutically acceptable salt thereof) is given at an effective dose.

With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alteration to treatment regimen. The dosing schedule can vary depending on a number of clinical factors, such as the subject's sensitivity to the drugs.

The present disclosure contemplates formulation of the subject compounds in any of the aforementioned pharmaceutical compositions and preparations. Furthermore, the present disclosure contemplates administration via any of the foregoing routes of administration. One of skill in the art can select the appropriate formulation and route of administration based on the condition being treated and the overall health, age, and size of the patient being treated.

### EXAMPLES

Examples are provided below to facilitate a more complete understanding of the compounds and methods disclosed herein. The following examples illustrate exemplary modes of making and practicing the compounds and methods disclosed herein. However, the scope of the disclosure is not limited to specific embodiments disclosed in these Examples, which are for purposes of illustration only, since alternative methods can be utilized to obtain similar results.

General. All oxygen or moisture sensitive reactions were carried out under N₂ atmosphere in glassware that was flame-dried under vacuum (0.5 mmHg) and purged with N₂ prior to use. All reagents and solvents were purchased from commercial vendors and used as received, or synthesized according to the footnoted references. NMR spectra were recorded on a Bruker 400 (400 MHz ¹H, 75 MHz ¹³C) or Varian (400 MHz ¹H, 75 MHz ¹³C) spectrometer. Proton and carbon chemical shifts are reported in ppm (δ) referenced to the NMR solvent. Data are reported as follows: chemical shifts, multiplicity (br=broad, s=singlet, t=triplet, q=quartet, m=multiplet; coupling constant (s) in Hz). Unless otherwise indicated NMR data were collected at 25° C. Flash chromatography was performed using 100-200 mesh Silica Gel. Liquid Chromatography/Mass Spectrometry (LCMS) was performed on Agilent 1200HPLC and 6110MS. Analytical thin layer chromatography (TLC) was performed on 0.2 mm silica gel plates. Visualization was accomplished with UV light and aqueous potassium permanganate (KMnO₄) stain followed by heating.

TABLE 2
Abbreviations

| | |
|---|---|
| 17AAG | 17-allylaminogeldanamycin |
| ACTH | adrenocorticotropin hormone |
| ALCL | anaplastic large cell lymphoma |
| ALS | amyotrophic lateral sclerosis |
| ALSD | amyotrophic lateral sclerosis with dementia |
| aq. | aqueous |
| B₂Pin₂ | bis(pinacolato)diboron |
| BPin | pinacolboronyl |
| Boc | t-butoxycarbonyl |
| BPO | benzoyl peroxide |
| CDI | carbonyldiimidazole |
| CNS | central nervous system |
| conc. | concentrated |
| DAPI | 4′,6-diamidino-2-phenylindole |
| DCC | N,N′-dicyclohexylcarbodiimide |
| DCE | 1,2-dichloroethane |
| DCM | dichloromethane |
| DIEA | N,N-diisopropylethylamine |
| DIPEA | N,N-diisopropylethylamine |
| DLBD | diffuse Lewy body disease |
| DMA | N,N-dimethylacetamide |
| DMAP | 4-dimethylaminopyridine |
| DME | 1,2-dimethoxyethane |
| DMF | N,N-dimethylformamide |
| DMOE | 1,2-dimethoxyethane |
| DMP | Dess-Martin periodinane |
| DMSO | dimethylsulfoxide |
| DPPA | diphenylphosphoryl azide |
| dppf | 1,1′-bis(diphenylphosphino)ferrocene |
| dtbppy | 4,4′-di-tert-butyl-2,2′-dipyridyl |
| EATL | enteropathy-associated intestinal T-cell lymphoma |
| EBV | Epstein-Barr virus |
| EDTA | ethylenediaminetetraacetic acid |
| ENKL | extranodal natural killer T-cell lymphoma nasal type |
| ESI | electrospray ionization |
| EtOAc, EA | ethyl acetate |
| FMRP, FMR1 | fragile X mental retardation protein |
| FSHD | facioscapulohumeral muscular dystrophy |
| FTD | frontotemporal dementia |
| FTDP-17 | Parkinsonism linked to chromosome 17 |
| FTLD | frontotemporal lobar dementia |
| FTLD-U | frontotemporal lobar dementia with ubiquitin inclusions |
| FUS | fused in sarcoma |
| G3BP | GTPase activating protein binding protein |
| HATU | O-(7-azabenzotriazol-1-yl)-N,N,N′,N′- |
| | tetramethyluronium hexafluorophosphate |
| HD | Huntington's disease |
| HIV | human immunodeficiency virus |
| ¹H-NMR | proton nuclear magnetic resonance |
| HOAc | acetic acid |
| HPLC | high performance liquid chromatography |
| IBMPFD | frontotemporal dementia with inclusion body myopathy |
| IPA | 2-propanol/isopropanol |
| LCMS | liquid chromatography-mass spectrum/spectrometry |
| MALT | mucosa-associated lymphoid tissue |
| MELAS | mitochondrial encephalomyopathy |
| MeOH | methanol |
| MS | mass spectrum/spectrometry |
| PE | petroleum ether |
| PFA | paraformaldehyde |
| PMA | progressive muscular atrophy |
| polyQ | polyglutamine |
| PPMA | post-polio muscular atrophy |
| PPS | post-polio syndrome |
| PSP | progressive supranuclear palsy |
| RSV | respiratory syncytial virus |
| rt | room temperature |
| SBMA | spinal and bulbar muscular atrophy |
| SG | stress granule |
| SMA | spinal muscular atrophy |
| STAB | sodium triacetoxyborohydride |
| T₃P | propanephosphonic acid anhydride |
| TBN | tert-Butyl nitrite |
| TEA | triethylamine |
| TFA | trifluoroacetic acid |
| THF | tetrahydrofuran |
| TIA-1 | T-cell intracellular antigen 1 |
| TIAR, TIAL1 | TIA1 cytotoxic granule-associated RNA binding protein- |
| | like 1 |
| TLC | thin layer chromatography |
| TTP, ZFP36 | tris tetraprolin |
| UV | ultraviolet |

### Example 1: Synthesis of (S)-(3-aminopyrrolidin-1-yl)(3-methyl-5-(4-(1-methylpiperidin-4-yl)-2-(trifluoromethoxy)phenyl)thiophen-2-yl)methanone (Compound 103)

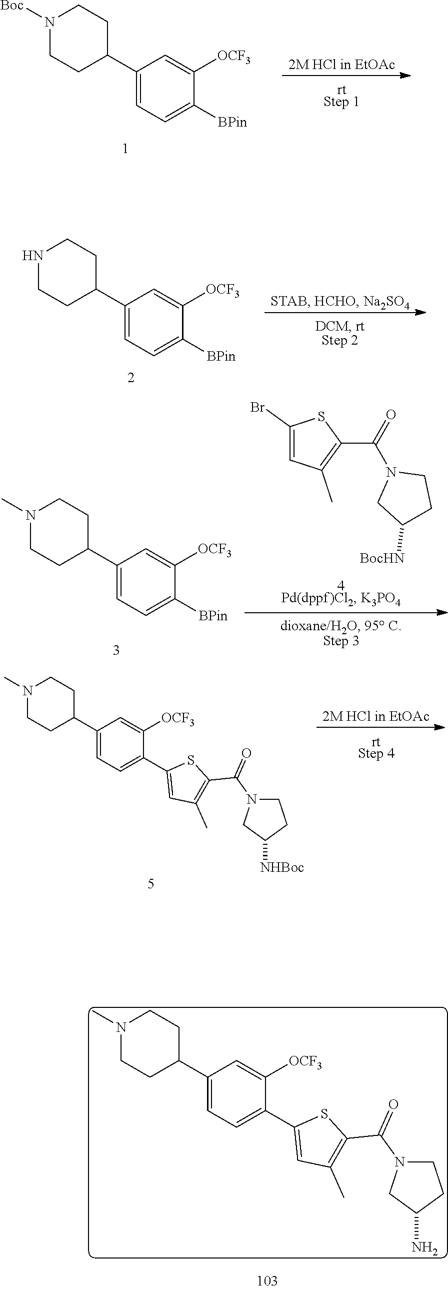

### Step 1 4-(4-(4,4,5,5-tetramethyl-1,3,2-dioxaborolan-2-yl)-3-(trifluoromethoxy)phenyl)piperidine

A mixture of tert-butyl 4-(4-(4,4,5,5-tetramethyl-1,3,2-dioxaborolan-2-yl)-3-(trifluoromethoxy)phenyl)piperidine-1-carboxylate (1.00 g, 2.10 mmol) in HCl (10 mL, 2M in EtOAc) was stirred at 25° C. for 3 hours. LCMS showed the reaction was completed. The reaction mixture was concentrated to dryness to afford the desired product (0.95 g, 95.2% yield) as a yellow solid.

### Step 2 1-methyl-4-(4-(4,4,5,5-tetramethyl-1,3,2-dioxaborolan-2-yl)-3-(trifluoromethoxy)phenyl)piperidine

To a mixture of compound 2 (950 mg, 2.56 mmol) in DCM (90 mL) were added NaBH(OAc)₃ (4.88 g, 23.03 mmol), HCHO (831 mg, 10.24 mmol, 37 wt % in H₂O) and Na₂SO₄ (182 mg, 1.28 mmol), then it was stirred at 25° C. for 12 hours. LCMS showed the reaction was completed. The reaction mixture was concentrated to dryness. The crude product was purified by column chromatography on silica gel (DCM/MeOH=10/1) to afford the desired product (850 mg, 77.6% yield) was obtained as a yellow solid.

### Step 3 tert-butyl (S)-(1-(3-methyl-5-(4-(1-methylpiperidin-4-yl)-2-(trifluoromethoxy)phenyl)thiophene-2-carbonyl)pyrrolidin-3-yl)carbamate

To a mixture of compound 3 (400 mg, 1.04 mmol) in 1,4-dioxane (8 mL) and H₂O (1 mL) were added compound 4 (364 mg, 0.93 mmol), K₃PO₄ (661 mg, 3.11 mmol) and Pd(dppf)Cl₂*DCM (127 mg, 0.16 mmol), then it was stirred at 95° C. under N₂ for 16 hours.

LCMS showed the reaction was completed. The reaction mixture was concentrated to dryness. The crude product was purified by column chromatography on silica gel (DCM/MeOH=10/1) to afford the desired product (160 mg, 25.8% yield) was obtained as yellow oil.

### Step 4 (S)-(3-aminopyrrolidin-1-yl)(3-methyl-5-(4-(1-methylpiperidin-4-yl)-2-(trifluoromethoxy)phenyl)thiophen-2-yl)methanone

A mixture of compound 5 (160 mg, 0.28 mmol) in HCl (3 mL, 2M in EtOAc) was stirred at 25° C. for 3 hours. LCMS showed the reaction was completed. The reaction mixture was concentrated to dryness. The crude product was purified by prep-HPLC to afford the desired product (Compound 103; 27.6 mg, 20.9% yield) was obtained as a white solid.

MS (ESI): mass calc'd. for C₂₃H₂₈F₃N₃O₂S 468.20, m/z found 468.2 [M+H]⁺.

¹H NMR (400 MHz, MeOD) 7.75 (d, J=8.0 Hz, 1H), 7.36 (dd, J=8.4, 1.6 Hz, 1H), 7.32 (s, 1H), 7.30 (s, 1H), 4.03-3.93 (m, 2H), 3.86-3.73 (m, 2H), 3.72-3.60 (m, 3H), 3.22-3.10 (m, 2H), 3.03-2.95 (m, 1H), 2.92 (s, 3H), 2.49-2.39 (m, 1H), 2.36 (s, 3H), 2.19-2.08 (m, 3H), 2.04-1.92 (m, 2H).

### Example 2: Synthesis of (S)-(3-aminopyrrolidin-1-yl)(3-methyl-5-(5-(1-methylpiperidin-4-yl)pyridin-2-yl)thiophen-2-yl)methanone (Compound 104)

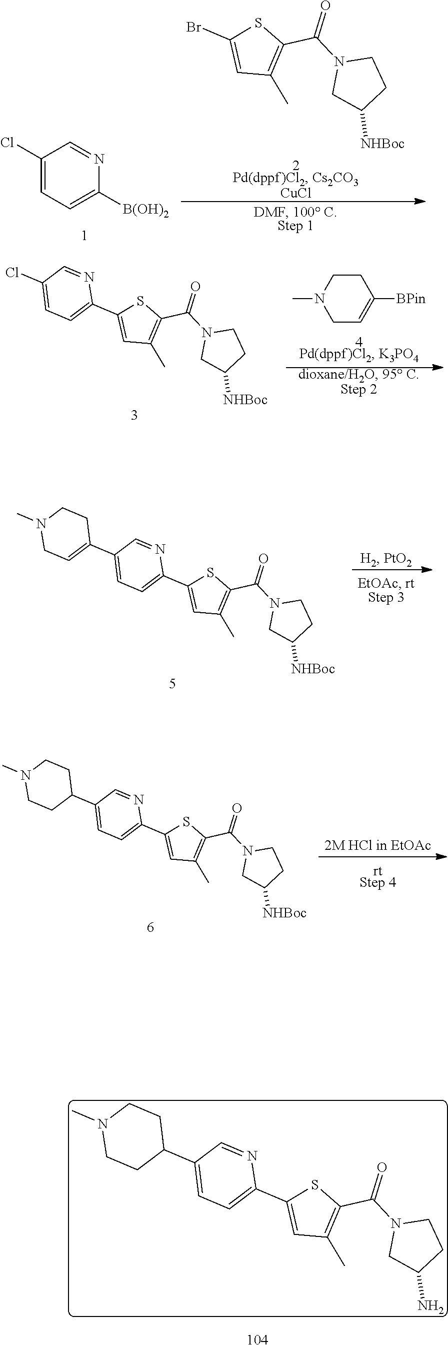

### Step 1 Synthesis of tert-butyl (S)-(1-(5-(5-chloropyridin-2-yl)-3-methylthiophene-2-carbonyl)pyrrolidin-3-yl)carbamate

To a solution of tert-butyl (S)-(1-(5-bromo-3-methylthiophene-2-carbonyl)pyrrolidin-3-yl)carbamate (390 mg, 1 mmol) in DMF (10 mL) were added (5-chloropyridin-2-yl)boronic acid (157.6 mg, 1 mmol), Cs₂CO₃ (979.2 mg, 3 mmol), Pd(dppf)Cl₂DCM (163.5 mg, 0.2 mmol) and CuCl (99 mg, 1 mmol). The mixture was stirred at 100° C. under N₂ for 16 h. After the reaction, the mixture was filtered and concentrated under reduced pressure. The residue was purified by flash chromatography PE:EA=1:1 to give tert-butyl (S)-(1-(5-(5-chloropyridin-2-yl)-3-methylthiophene-2-carbonyl)pyrrolidin-3-yl)carbamate (85 mg, 19.11% yield) as a yellow solid.

LCMS (ESI) calc'd for C₂₀H₂₅ClN₃O₃S+ [M+H]+ m/z 422.13, found 422.

### Step 2 Synthesis of tert-butyl (S)-(1-(3-methyl-5-(1′-methyl-1′,2′,3′,6′-tetrahydro-[3,4′-bipyridin]-6-yl)thiophene-2-carbonyl)pyrrolidin-3-yl)carbamate

To a solution of tert-butyl (S)-(1-(5-(5-chloropyridin-2-yl)-3-methylthiophene-2-carbonyl)pyrrolidin-3-yl)carbamate (430 mg, 1.02 mmol) in dioxane/H₂O=8:1 (18 mL) were added 1-methyl-4-(4,4,5,5-tetramethyl-1,3,2-dioxaborolan-2-yl)-3,6-dihydro-2H-pyridine (227.4 mg, 1.02 mmol), K₃PO₄ (649 mg, 3.06 mmol) and Pd(dppf)Cl₂DCM (166.3 mg, 0.2 mmol). The mixture was stirred at 95° C. under N₂ for 16 h. After the reaction, the mixture was filtered and concentrated under reduced pressure. The residue was purified by flash chromatography (MeOH:EA=2:1) to give tert-butyl (S)-(1-(3-methyl-5-(1′-methyl-1′,2′,3′,6′-tetrahydro-[3,4′-bipyridin]-6-yl)thiophene-2-carbonyl)pyrrolidin-3-yl)carbamate (200 mg, 32.5% yield) as a brown solid. LCMS (ESI) calc'd for C₂₆H₃₄N₄O₃S+ [M+H]+ m/z 482.24, found 483.

### Step 3 Synthesis of tert-butyl (S)-(1-(3-methyl-5-(5-(1-methylpiperidin-4-yl)pyridin-2-yl)thiophene-2-carbonyl)pyrrolidin-3-yl)carbamate

A solution of tert-butyl (S)-(1-(3-methyl-5-(1′-methyl-1′,2′,3′,6′-tetrahydro-[3,4′-bipyridin]-6-yl)thiophene-2-carbonyl)pyrrolidin-3-yl)carbamate (448 mg, 0.88 mmol) and PtO2 (19.9 mg, 0.088 mmol) in EA (20 mL) was stirred at rt under H₂ for 16 h. The LCMS showed the reaction was completed and the desired MS was found. The resulting mixture was filtered and concentrated under reduced pressure to give tert-butyl (S)-(1-(3-methyl-5-(5-(1-methylpiperidin-4-yl)pyridin-2-yl)thiophene-2-carbonyl)pyrrolidin-3-yl)carbamate (380 mg, 80.3% yield) as a yellow oil. LCMS (ESI) calc'd for C₂₈H₄₀N₃O₄S+ [M+H]+ m/z 514.28, found 514.

### Step 4 Synthesis of (S)-(3-aminopyrrolidin-1-yl)(3-methyl-5-(5-(1-methylpiperidin-4-yl)pyridin-2-yl)thiophen-2-yl)methanone

To a solution of tert-butyl (S)-(1-(3-methyl-5-(5-(1-methylpiperidin-4-yl)pyridin-2-yl)thiophene-2-carbonyl)pyrrolidin-3-yl)carbamate (220 mg, 0.45 mmol) in 4 mL EtOAc was added HCl (3 mL, 2M in EtOAc). The mixture was stirred at rt for 16 h. The LCMS showed the reaction was completed and the desired product was found. The mixture was concentrated under reduced pressure. The residue was purified by prep-HPLC to give (S)-(3-aminopyrrolidin-1-yl)(3-methyl-5-(5-(1-methylpiperidin-4-yl)pyridin-2-yl)thiophen-2-yl)methanone (Compound 104; 34.7 mg, 19.7% yield) as a yellow solid.

LCMS (ESI) calc'd for C₂₂H₃₀N₃O₂S+ [M+H]+ m/z 400.21, found 400.

¹H NMR (400 MHz, MeOD) δ 8.89 (d, J=2.1 Hz, 2H), 8.24 (dd, J=8.3, 2.2 Hz, 1H), 7.59 (d, J=8.3 Hz, 1H), 7.43 (s, 1H), 4.04-3.95 (m, 2H), 3.84 (dt, J=11.7, 7.5 Hz, 1H), 3.75 (ddd, J=17.7, 10.6, 5.8 Hz, 2H), 3.66 (s, 2H), 3.22 (dd, J=8.1, 3.1 Hz, 1H), 3.20-3.13 (m, 1H), 2.96 (d, J=15.5 Hz, 3H), 2.45 (td, J=14.0, 7.5 Hz, 1H), 2.37 (s, 3H), 2.17 (ddt, J=22.6, 14.6, 8.5 Hz, 5H).

### Example 3: Synthesis of (S)-(3-aminopyrrolidin-1-yl)(3-methyl-5-(6-(1-methylpiperidin-4-yl)pyridin-3-yl)thiophen-2-yl)methanone (Compound 108)

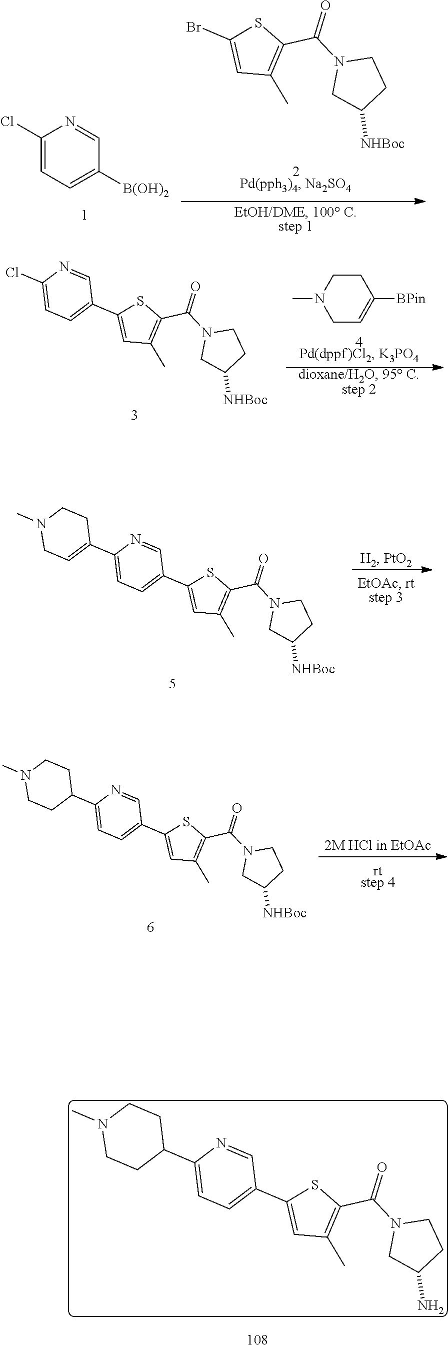

### Step 1 tert-butyl(S)-(1-(5-(6-chloropyridin-3-yl)-3-methylthiophene-2-carbonyl)pyrrolidin-3-yl)carbamate

To the solution of (6-chloropyridin-3-yl)boranediol (230 mg, 1.4616 mmol) in EtOH/DME=1:1 (8 mL) were added tert-butyl (S)-(1-(5-bromo-3-methylthiophene-2-carbonyl)pyrrolidin-3-yl)carbamate (569 mg, 1.4616 mmol), Na₂CO₃ (464.8 mg, 4.3848 mmol) and Pd(PPh₃)₄ (337.8 mg, 0.2923 mmol). The mixture was stirred under N₂ at 95° C. for 16 h. After the reaction, the mixture was cooled to room temperature, the mixture was filtered and concentrated under reduced pressure. The residue was purified by silica gel column chromatography to give the desired product (520 mg, 75.89% yield) as a yellow solid.

LCMS (ESI) calc'd for C11H14ClNO4S+ [M+H]+ m/z 422, found 422.

### Step 2 tert-butyl(S)-(1-(3-methyl-5-(1′-methyl-1′,2′,3′,6′-tetrahydro-(2,4′-bipyridin)-5-yl)thiophene-2-carbonyl)pyrrolidin-3-yl)carbamate

To the solution of tert-butyl(S)-(1-(5-(6-chloropyridin-3-yl)-3-methylthiophene-2-carbonyl)pyrrolidin-3-yl)carbamate (520 mg, 1.2324 mmol) in dioxane/H₂O=8:1 (18 mL) were added 1-methyl-4-(4,4,5,5-tetramethyl-1,3,2-dioxaborolan-2-yl)-3,6-dihydro-2H-pyridine (412.5 mg, 1.8486 mmol), K₃PO₄ (915.6 mg, 4.3134 mmol) and Pd(dppf)Cl₂DCM (201.1 mg, 0.2464 mmol). The mixture was stirred at 95° C. under N₂ for 16 h. After the reaction, the mixture was cooled to room temperature, the mixture was filtered and concentrated under reduced pressure. The residue was purified by silica gel column chromatography to give the desired product (210 mg, 31.78% yield) as a yellow solid. LCMS (ESI) calc'd for C₂₆H₃₅N₄O₃S+ [M+H]+ m/z 483, found 483.

### Step 3 tert-butyl(S)-(1-(3-methyl-5-(6-(1-methylpiperidin-4-yl)pyridin-3-yl)thiophene-2-carbonyl)pyrrolidin-3-yl)carbamate

To the solution of tert-butyl(S)-(1-(3-methyl-5-(1′-methyl-1′,2′,3′,6′-tetrahydro-(2,4′-bipyridin)-5-yl)thiophene-2-carbonyl)pyrrolidin-3-yl)carbamate (210 mg, 0.4351 mmol) in EtOAc (5 mL) was added PtO₂ (19.7 mg). The mixture was stirred at rt under H₂ for 16 h. After the reaction, the mixture was filtered and concentrated under reduced pressure to afford the desired product (200 mg, 85.36% yield) as a yellow solid. LCMS (ESI) calc'd for C26H37N4O3S+ [M+H]+ m/z 485, found 485.

### Step 4 (S)-(3-aminopyrrolidin-1-yl)(3-methyl-5-(6-(1-methylpiperidin-4-yl)pyridin-3-yl)thiophen-2-yl)methanone

To the solution of tert-butyl(S)-(1-(3-methyl-5-(6-(1-methylpiperidin-4-yl)pyridin-3-yl)thiophene-2-carbonyl)pyrrolidin-3-yl)carbamate (100 mg, 0.2063 mmol) in EtOAc (2 mL) was added HCl (5 ml, 2M in EtOAc). The mixture was stirred at rt for 16 h. After the reaction, the resulting mixture was concentrated under reduced pressure. The residue was purified by prep-HPLC to give the desired product (Compound 108; 15 mg, 17.01% yield) as a yellow solid. LCMS (ESI) calc'd for C21H29N4OS+ [M+H]+ m/z 385, found 385. ¹H NMR (400 MHz, MeOD) δ 8.82 (d, J=2.0 Hz, 1H), 8.06-8.05 (m, 1H), 7.43 (d, J=8.0 Hz, 1H), 7.39 (s, 1H), 4.05-3.98 (m, 2H), 3.88-3.77 (m, 2H), 3.74-3.63 (m, 3H), 3.25-3.05 (m, 3H), 2.96 (d, J=8.0 Hz, 3H), 2.47 (d, J=7.6 Hz, 1H), 2.39 (s, 3H), 2.16-2.10 (m, 5H).

### Example 4: Synthesis of (S)-(3-allyl-5-(4-(1-isopropylpiperidin-4-yl)phenyl)thiophen-2-yl)(3-aminopyrrolidin-1-yl)methanone (Compound 109)

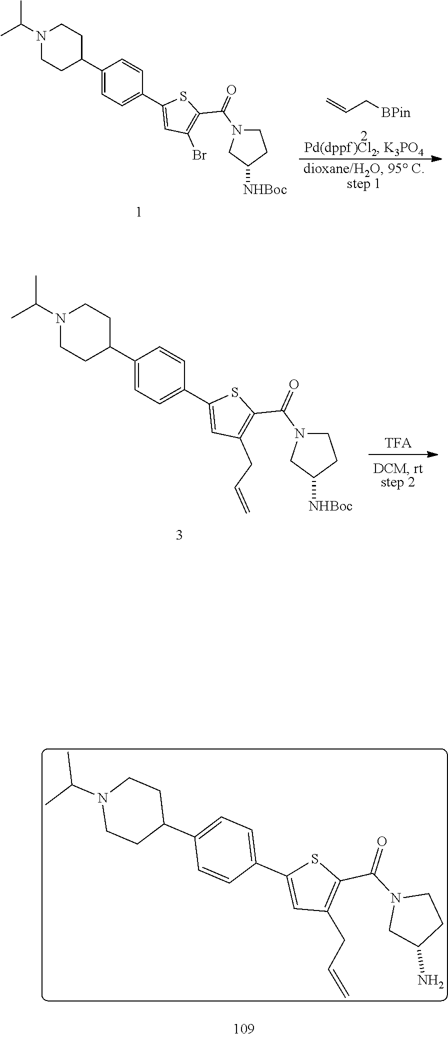

### Step 1 tert-butyl(S)-(1-(3-allyl-5-(4-(1-isopropylpiperidin-4-yl)phenyl)thiophene-2-carbonyl)pyrrolidin-3-yl)carbamate

To the solution of tert-butyl (S)-(1-(3-bromo-5-(4-(1-isopropylpiperidin-4-yl)phenyl)thiophene-2-carbonyl)pyrrolidin-3-yl)carbamate (650 mg, 1.1273 mmol) in dioxane/H₂O=8:1 (18 mL) were added 4,4,5,5-tetramethyl-2-(prop-2-en-1-yl)-1,3,2-dioxaborolane (189.4 mg, 1.1273 mmol), K₃PO₄ (717.8 mg, 3.3819 mmol) and Pd(dppf)Cl₂DCM (91.9 mg, 0.1127 mmol). The mixture was stirred at 95° C. under N₂ for 16 h. After the reaction, the mixture was cooled to room temperature, the mixture was filtered and concentrated under reduced pressure. The residue was purified by silica gel column chromatography to give the desired product (390 mg, 57.90% yield) as a yellow solid. LCMS (ESI) calc'd for C31H44N3O₃S+ [M+H]+ m/z 538, found 538.

### Step 2 (S)-(3-allyl-5-(4-(1-isopropylpiperidin-4-yl)phenyl)thiophen-2-yl)(3-aminopyrrolidin-1-yl)methanone

To the solution of tert-butyl(S)-(1-(3-allyl-5-(4-(1-isopropylpiperidin-4-yl)phenyl)thiophene-2-carbonyl)pyrrolidin-3-yl)carbamate (150 mg, 0.2789 mmol) in EtOAc (2 mL) was added 2M HCl in EtOAc (5 mL). The mixture was stirred at rt for 16 h. After the reaction, the resulting mixture was concentrated under reduced pressure. The residue was purified by prep-HPLC to give the desired product (Compound 109; 30 mg, 22.12% yield) as a yellow solid. LCMS (ESI) calc'd for C26H36N3OS+ [M+H]+ m/z 438, found 438. ¹H NMR (400 MHz, MeOD) δ 7.64 (d, J=8.4 Hz, 2H), 7.35 (d, J=8.4 Hz, 2H), 7.28 (s, 1H), 6.02 (m, 1H), 5.21-5.07 (m, 2H), 4.06-3.94 (m, 2H), 3.88-3.65 (m, 3H), 3.58 (m, 3H), 3.52 (d, J=6.4 Hz, 2H), 3.23 (m, 2H), 3.03-2.91 (m, 1H), 2.45 (m, 1H), 2.23-1.97 (m, 5H), 1.43 (d, J=6.8 Hz, 6H).

### Example 5: Synthesis of (S)-(3-aminopyrrolidin-1-yl)(5-(4-(1-isopropylpiperidin-4-yl)phenyl)-3-propylthiophen-2-yl)methanone (Compound 110)

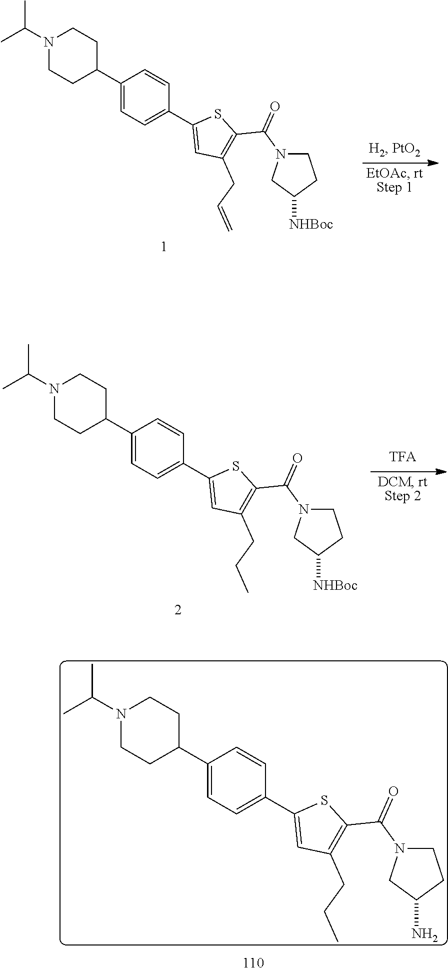

### Step 1 tert-butyl(S)-(1-(5-(4-(1-isopropylpiperidin-4-yl)phenyl)-3-propylthiophene-2-carbonyl)pyrrolidin-3-yl)carbamate

To the solution of tert-butyl (S)-(1-(3-allyl-5-(4-(1-isopropylpiperidin-4-yl)phenyl)thiophene-2-carbonyl)pyrrolidin-3-yl)carbamate (150 mg, 0.2789 mmol) in EtOAc (5 mL) was added PtO₂ (12.7 mg). The mixture was stirred at rt under H₂ for 16 h. The LCMS showed the reaction was completed and the desired MS was found. The solvent was removed and the residue was purified by flash chromatography to give the desired product as a yellow solid. MS (ESI): mass calc'd for C31H45N3O3S 539, m/z found 540 [M+H]+.

### Step 2 (S)-(3-aminopyrrolidin-1-yl)(5-(4-(1-isopropylpiperidin-4-yl)phenyl)-3-propylthiophen-2-yl)methanone

To the solution of tert-butyl(S)-(1-(5-(4-(1-isopropylpiperidin-4-yl)phenyl)-3-propylthiophene-2-carbonyl)pyrrolidin-3-yl)carbamate (150 mg, 0.2779 mmol) in EtOAc (2 mL) was added HCl (5 mL, 2M in EtOAc). The mixture was stirred at rt for 16 h. The LCMS showed the reaction was completed and the desired MS was found. The solvent was removed under reduced pressure and the residue was purified by prep-HPLC to give the desired product (Compound 110; 30 mg, 22.09% yield) as a yellow solid. MS (ESI): mass calc'd C26H38N3OS 440, m/z found 440 [M+H]+. ¹H NMR (400 MHz, MeOD) δ 7.68-7.60 (m, 2H), 7.35 (d, J=8.4 Hz, 2H), 7.32 (s, 1H), 4.05-3.95 (m, 2H), 3.89-3.65 (m, 3H), 3.58 (m, 3H), 3.23 (m, 2H), 3.02-2.92 (m, 1H), 2.75-2.67 (m, 2H), 2.46 (m, 1H), 2.24-1.97 (m, 5H), 1.72 (m, 2H), 1.43 (d, J=6.8 Hz, 6H), 0.99 (t, J=7.2 Hz, 3H).

### Example 6: Synthesis of (S)-(3-aminopyrrolidin-1-yl)(5-(3-methoxy-4-(1-methylpiperidin-4-yl)phenyl)-3-methylthiophen-2-yl)methanone (Compound 111)

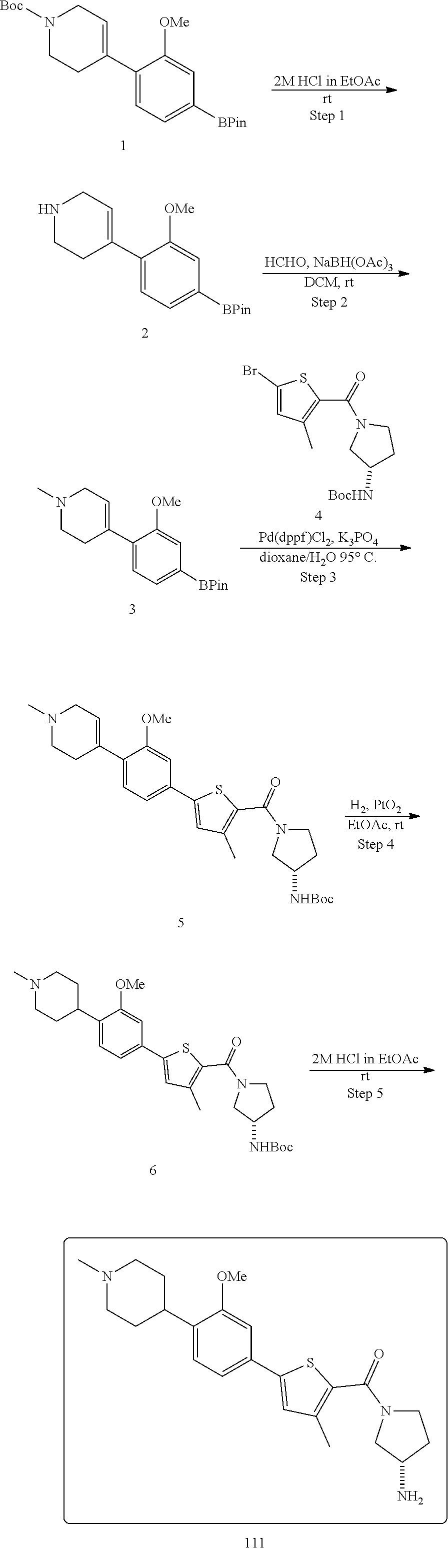

### Step 1 4-(2-methoxy-4-(4,4,5,5-tetramethyl-1,3,2-dioxaborolan-2-yl)phenyl)-1,2,3,6-tetrahydropyridine

A solution of tert-butyl 4-(2-methoxy-4-(4,4,5,5-tetramethyl-1,3,2-dioxaborolan-2-yl)phenyl)-3,6-dihydropyridine-1(2H)-carboxylate (580 mg, 1.4 mmol) in 4 mL EtOAc was added HCl (5 mL, 2M in EA). The mixture was stirred at rt for 16 h. The LCMS showed the reaction was completed and the desired MS was found. the resulting mixture was concentrated under reduced pressure to give 4-(2-methoxy-4-(4,4,5,5-tetramethyl-1,3,2-dioxaborolan-2-yl)phenyl)-1,2,3,6-tetrahydropyridine (509 mg, 110% yield) as a yellow oil. LCMS (ESI) calc'd for C18H27BNO3+ [M+H]+ m/z 316.21, found 316.

### Step 2 4-(2-methoxy-4-(4,4,5,5-tetramethyl-1,3,2-dioxaborolan-2-yl)phenyl)-1-methyl-1,2,3,6-tetrahydropyridine

To a solution of 4-(2-methoxy-4-(4,4,5,5-tetramethyl-1,3,2-dioxaborolan-2-yl)phenyl)-1,2,3,6-tetrahydropyridine (1100 mg, 3.5 mmol) in DCM (20 mL) was added HCHO (314.4 mg, 10.5 mmol, 37 wt % in H₂O) and NaBH(OAc)₃ (440 mg, 10.5 mmol). The mixture was stirred at rt for 16 h. The LCMS showed the reaction was completed and the desired MS was found. The residue was concentrated under reduced pressure to give 4-(2-methoxy-4-(4,4,5,5-tetramethyl-1,3,2-dioxaborolan-2-yl)phenyl)-1-methyl-1,2,3,6-tetrahydropyridine (900 mg, 74.4% yield) as a yellow solid. LCMS (ESI) calc'd for C19H29BNO3+ [M+H]+ m/z 330.23, found 330.

### Step 3 (S)-(1-(5-(3-methoxy-4-(1-methyl-1, 2, 3,6-tetrahydropyridin-4-yl)phenyl)-3-methylthiophene-2-carbonyl)pyrrolidin-3-yl)carbamate

To a solution of tert-butyl (S)-(1-(5-bromo-3-methylthiophene-2-carbonyl)pyrrolidin-3-yl)carbamate (800 mg, 2.05 mmol) in dioxane/H₂O=8:1 (18 mL) were added 4-(2-methoxy-4-(4,4,5,5-tetramethyl-1,3,2-dioxaborolan-2-yl)phenyl)-1-methyl-1,2,3,6-tetrahydropyridine (676.6 mg, 2.05 mmol), K₃PO₄ (1308.6 mg, 6.16 mmol) and Pd(dppf)Cl₂DCM (335.4 mg, 0.41 mmol). The mixture was stirred at 95° C. under N₂ for 16 h.

After cooling rt, the mixture was filtered and concentrated under reduced pressure. The residue was purified by flash chromatography using MeOH:EA=1:10 as eluent to afford tert-butyl (S)-(1-(5-(3-methoxy-4-(1-methyl-1,2,3,6-tetrahydropyridin-4-yl)phenyl)-3-methylthiophene-2-carbonyl)pyrrolidin-3-yl)carbamate (448 mg, 40.5% yield) as a yellow solid. LCMS (ESI) calc'd for C28H37N3O4S+ [M+H]+ m/z 511.25, found 512.

### Step 4 tert-butyl (S)-(1-(5-(3-methoxy-4-(1-methylpiperidin-4-yl)phenyl)-3-methylthiophene-2-carbonyl)pyrrolidin-3-yl)carbamate

A solution of tert-butyl (S)-(1-(5-(3-methoxy-4-(1-methyl-1,2,3,6-tetrahydropyridin-4-yl)phenyl)-3-methylthiophene-2-carbonyl)pyrrolidin-3-yl)carbamate (448 mg, 0.88 mmol) and PtO₂ (19.9 mg, 0.088 mmol) in EtOAc (20 mL) was stirred at rt under H₂ for 16 h. The LCMS showed the reaction was completed and the desired MS was found. The resulting mixture was filtered and concentrated under reduced pressure to give tert-butyl (S)-(1-(5-(3-methoxy-4-(1-methylpiperidin-4-yl)phenyl)-3-methylthiophene-2-carbonyl)pyrrolidin-3-yl)carbamate (380 mg, 80.3% Yield) as a yellow oil. LCMS (ESI) calc'd for C28H39N3O4S+ [M+H]+ m/z 513.27, found 514.

### Step 5 (S)-(3-aminopyrrolidin-1-yl)(5-(3-methoxy-4-(1-methylpiperidin-4-yl)phenyl)-3-methylthiophen-2-yl)methanone

A solution of tert-butyl (S)-(1-(5-(3-methoxy-4-(1-methylpiperidin-4-yl)phenyl)-3-methylthiophene-2-carbonyl)pyrrolidin-3-yl)carbamate (380 mg, 0.74 mmol) in 4 mL EtOAc was added HCl (5 mL, 2M in EtOAc). The mixture was stirred at rt for 16 h. The LCMS showed the reaction was completed and the desired MS was found. The resulting mixture was concentrated under reduced pressure to give (S)-(3-aminopyrrolidin-1-yl)(5-(3-methoxy-4-(1-methylpiperidin-4-yl)phenyl)-3-methylthiophen-2-yl)methanone (Compound 111; 350 mg) as a yellow solid. LCMS (ESI) calc'd for C23H32N3O2S+ [M+H]+ m/z 414.22, found 414. ¹H NMR (400 MHz, MeOD) δ 7.30 (s, 1H), 7.24 (s, 2H), 7.22 (s, 1H), 4.01 (t, J=7.2 Hz, 2H), 3.94 (d, J=7.1 Hz, 3H), 3.87 (dt, J=9.2, 6.2 Hz, 1H), 3.83-3.69 (m, 2H), 3.63 (d, J=12.3 Hz, 2H), 3.25-3.12 (m, 2H), 2.93 (s, 3H), 2.52-2.42 (m, 1H), 2.38 (s, 3H), 2.23-1.92 (m, 6H).

### Example 7: Synthesis of (S)-(3-aminopyrrolidin-1-yl)(3-methyl-5-(4-(1-methylpiperidin-4-yl)-3-(trifluoromethoxy)phenyl)thiophen-2-yl)methanone (Compound 112)

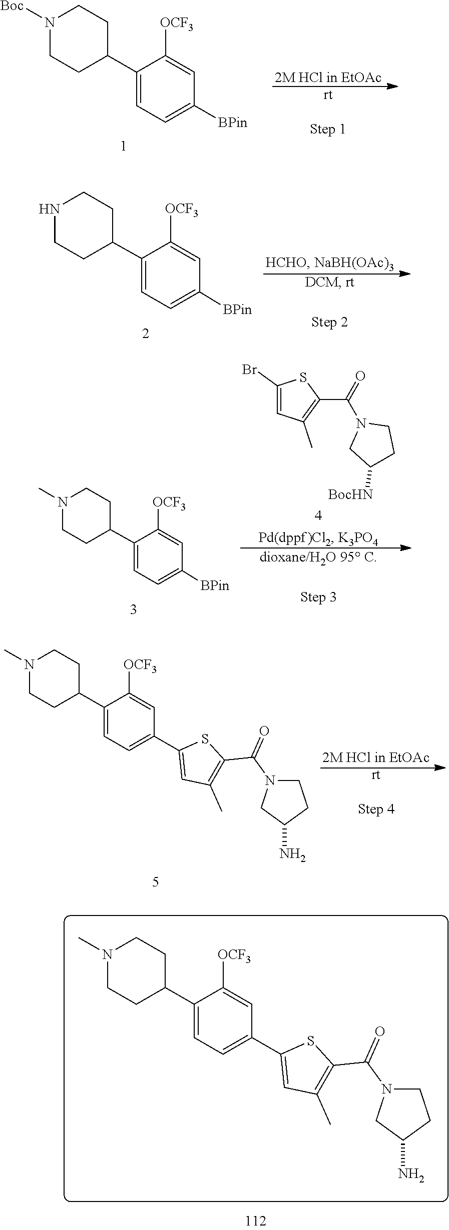

### Step 1 4-(4-(4,4,5,5-tetramethyl-1,3,2-dioxaborolan-2-yl)-2-(trifluoromethoxy)phenyl)piperidine

A solution of tert-butyl 4-(4-(4,4,5,5-tetramethyl-1,3,2-dioxaborolan-2-yl)-2-(trifluoromethoxy)phenyl)piperidine-1-carboxylate (470 mg, 1.00 mmol) in HCl (5 mL, 2M in EtOAc) was stirred at 25° C. for 3 hours. LCMS showed the reaction was completed. The reaction mixture was concentrated to dryness to afford the desired product (445 mg, 96.4% yield) as a yellow solid.

### Step 2 1-methyl-4-(4-(4,4,5,5-tetramethyl-1,3,2-dioxaborolan-2-yl)-2-(trifluoromethoxy)phenyl)piperidine

To a mixture of compound 2 (445 mg, 1.20 mmol) in DCM (45 mL) was added NaBH(OAc)₃ (2.29 g, 10.79 mmol), HCHO (389 mg, 4.80 mmol) and Na₂SO₄ (85 mg, 0.60 mmol), then it was stirred at 25° C. for 12 hours. LCMS showed the reaction was completed. The reaction mixture was concentrated to dryness. Then it was diluted with ethyl acetate and filtrated. The filtrate was concentrated to dryness to afford the desired product (450 mg, 87.7% yield) as yellow oil.

### Step 3 tert-butyl (S)-(1-(3-methyl-5-(4-(1-methylpiperidin-4-yl)-3-(trifluoromethoxy)phenyl)thiophene-2-carbonyl)pyrrolidin-3-yl)carbamate

To a mixture of compound 3 (450 mg, 1.17 mmol) in 1,4-dioxane (10 mL) and H₂O (1.2 mL) were added compound 4 (409 mg, 1.05 mmol), K₃PO₄ (620 mg, 2.92 mmol) and Pd(dppf)Cl₂*DCM (143 mg, 0.18 mmol), then it was stirred at 95° C. under N₂ for 16 hours.

LCMS showed the reaction was completed. The reaction mixture was concentrated to dryness. The crude product was purified by column chromatography on silica gel (MeOH/DCM=8%) to afford the desired product (280 mg, 38.0% yield) as a black solid.

### Step 4 (S)-(3-aminopyrrolidin-1-yl)(3-methyl-5-(4-(1-methylpiperidin-4-yl)-3-(trifluoromethoxy)phenyl)thiophen-2-yl)methanone

A solution of compound 5 (280 mg, 0.49 mmol) in HCl (3 mL, 2M in EtOAc) was stirred at 25° C. for 3 hours. LCMS showed the reaction was completed. The reaction mixture was concentrated to dryness. The crude product was purified by prep-HPLC to afford the desired product (Compound 112; 22.4 mg, 9.6% yield) as a white solid. MS (ESI): mass calc'd. for C₂₃H₂₉F₃N₃O₂S 468.20, m/z found 468.2 [M+H]+. ¹H NMR (400 MHz, MeOD) δ 7.65 (d, J=2.0 Hz, 1H), 7.56 (dd, J=8.0, 1.6 Hz, 1H), 7.39 (d, J=8.4 Hz, 1H), 7.26 (s, 1H), 3.82-3.72 (m, 2H), 3.68-3.54 (m, 2H), 3.32 (s, 1H), 3.10-3.00 (m, 3H), 2.35 (s, 3H), 2.33 (s, 3H), 2.26-2.15 (m, 3H), 1.92-1.82 (m, 3H), 1.81-1.72 (m, 2H).

### Example 8: Synthesis of (S)-(3-aminopyrrolidin-1-yl)(5-(4-(1-isopropylpiperidin-4-yl)phenyl)-3-vinylthiophen-2-yl)methanone (Compound 117)

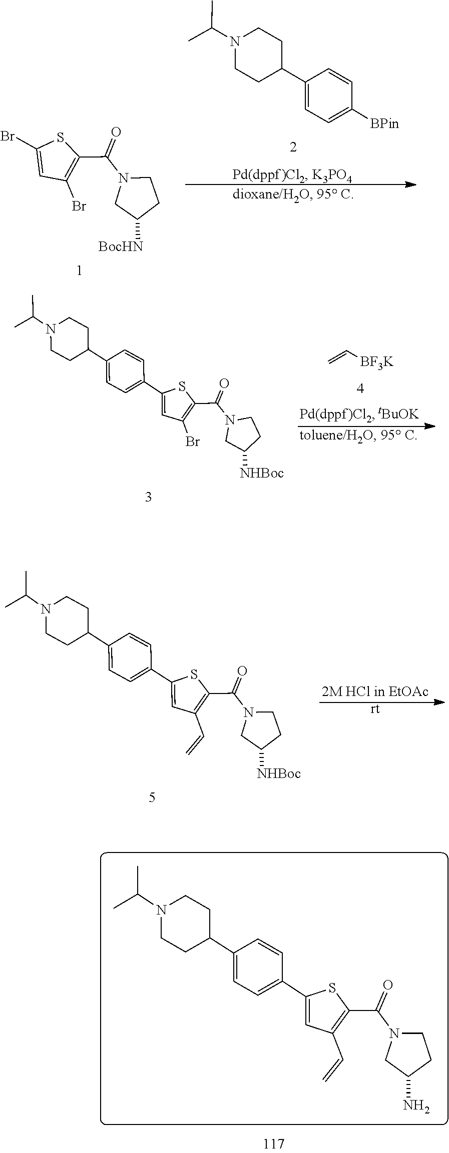

### Step 1 tert-butyl(S)-(1-(3-bromo-5-(4-(1-isopropylpiperidin-4-yl)phenyl)thiophene-2-carbonyl)pyrrolidin-3-yl)carbamate

To the solution of tert-butyl (S)-(1-(3,5-dibromothiophene-2-carbonyl)pyrrolidin-3-yl)carbamate (1 g, 0.0022 mol) in dioxane/H₂O=8:1 (18 mL) were added 1-isopropyl-4-(4-(4,4,5,5-tetramethyl-1,3,2-dioxaborolan-2-yl)phenyl)piperidine (0.72 g, 0.0022 mmol), K₃PO₄ (1.4 g, 0.0066 mol) and Pd(dppf)Cl₂DCM (0.18 g, 0.0002 mol). The mixture was stirred at 95° C. under N₂ for 16 h. The LCMS showed the reaction was completed and the desired MS was found. After cooled to rt, the mixture was filtered and concentrated under reduced pressure. The residue was purified by silica gel column chromatography to give the desired product (0.8 g, 54.55% yield) as a yellow solid. MS (ESI): mass calc'd for C28H39BrN3O3S 576, m/z found 576 [M+H]+.

### Step 2 tert-butyl(S)-(1-(5-(4-(1-isopropylpiperidin-4-yl)phenyl)-3-vinylthiophene-2-carbonyl)pyrrolidin-3-yl)carbamate

To a solution of tert-butyl (S)-(1-(3-bromo-5-(4-(1-isopropylpiperidin-4-yl)phenyl)thiophene-2-carbonyl)pyrrolidin-3-yl)carbamate (800 mg, 1.3875 mmol) in 10 mL of toluene and 1 mL of H₂O were added Potassium vinyltrifluoroborate (205.98 mg, 1.5262 mmol), t-BuOK (504.54 mg, 4.1625 mmol) and Pd(dppf)Cl₂DCM (113.22 mg, 0.1387 mmol). The mixture was stirred at 95° C. under N₂ for 16 hours. After the reaction, the reaction mixture was cooled to room temperature. The solvent was removed under reduced pressure and the residue was purified by silica gel column chromatography to give the desired product (500 mg, 61.69% yield) as a yellow oil. MS (ESI): mass calc'd for C30H42N3O3S 524, m/z found 524 [M+H]+.

### Step 3 (S)-(3-aminopyrrolidin-1-yl)(5-(4-(1-isopropylpiperidin-4-yl)phenyl)-3-vinylthiophen-2-yl)methanone

To the solution of tert-butyl(S)-(1-(5-(4-(1-isopropylpiperidin-4-yl)phenyl)-3-vinylthiophene-2-carbonyl)pyrrolidin-3-yl)carbamate (140 mg, 0.2673 mmol) in EtOAc (2 mL) was added HCl (4 mL, 2M in EtOAc). The mixture was stirred at rt for 16 h. The LCMS showed the reaction was completed and the desired MS was found. The solvent was removed under reduced pressure and the residue was purified by prep-HPLC to give the desired product (Compound 117; 20 mg, 15.90% yield) as a white solid. MS (ESI): mass calc'd for C25H34N3OS 424, m/z found 424 [M+H]+. 1H NMR (400 MHz, MeOD) δ 7.72-7.64 (m, 3H), 7.37 (d, J=8.4 Hz, 2H), 6.84 (m, 1H), 5.88 (m, 1H), 5.44 (m, 1H), 3.99 (d, J=10.8 Hz, 2H), 3.86-3.64 (m, 3H), 3.59 (m, 3H), 3.23 (m, 2H), 2.98 (m, 1H), 2.46 (m, 1H), 2.24-1.97 (m, 5H), 1.43 (d, J=6.8 Hz, 6H).

### Example 9: Synthesis of (S)-(3-aminopyrrolidin-1-yl)(3-ethyl-5-(4-(1-isopropylpiperidin-4-yl)phenyl)thiophen-2-yl)methanone (Compound 118)

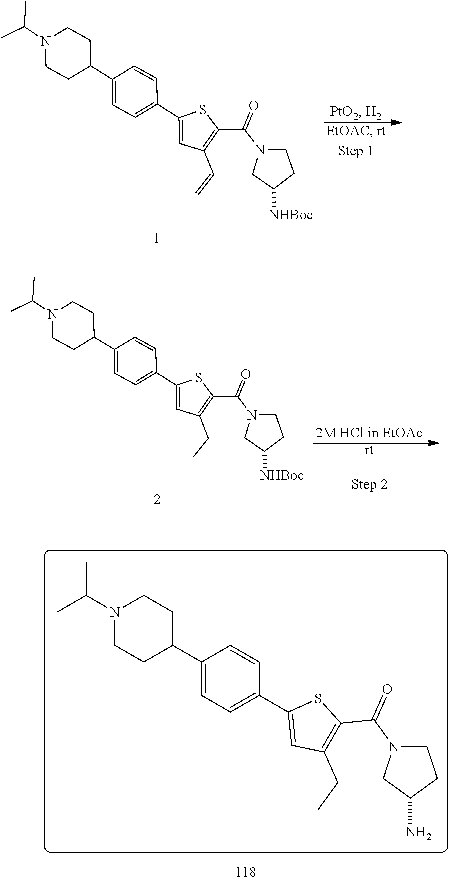 [SMILES: C=Cc1cc(-c2ccc(C3CCN(C(C)C)CC3)cc2)sc1C(=O)N1CC[C@H](NC(=O)OC(C)(C)C)C1 ; CCc1cc(-c2ccc(C3CCN(C(C)C)CC3)cc2)sc1C(=O)N1CC[C@H](NC(=O)OC(C)(C)C)C1 ; CCc1cc(-c2ccc(C3CCN(C(C)C)CC3)cc2)sc1C(=O)N1CC[C@H](N)C1]

### Step 1 tert-butyl(S)-(1-(3-ethyl-5-(4-(1-isopropylpiperidin-4-yl)phenyl)thiophene-2-carbonyl)pyrrolidin-3-yl)carbamate

To the solution of tert-butyl (S)-(1-(5-(4-(1-isopropylpiperidin-4-yl)phenyl)-3-vinylthiophene-2-carbonyl)pyrrolidin-3-yl)carbamate (350 mg, 0.6683 mmol) in EtOAc (5 mL) was added PtO₂ (30.35 mg). The mixture was stirred at rt under H₂ for 16 h. The LCMS showed the reaction was completed and the desired MS was found. The residue was purified by flash chromatography to give the desired product (300 mg, 76.85% yield) as a yellow solid.

MS (ESI): mass calc'd for C30H44N3O3S 526, m/z found 526 [M+H]+.

### Step 2 (S)-(3-aminopyrrolidin-1-yl)(3-ethyl-5-(4-(1-isopropylpiperidin-4-yl)phenyl)thiophen-2-yl)methanone

To the solution of tert-butyl (S)-(1-(3-ethyl-5-(4-(1-isopropylpiperidin-4-yl)phenyl)thiophene-2-carbonyl)pyrrolidin-3-yl)carbamate (300 mg, 0.5706 mmol) in EtOAc (2 mL) was added 2M HCl in EtOAc (5 ml). The mixture was stirred at rt for 16 h. The LCMS showed the reaction was completed and the desired MS was found. The residue was purified by prep-HPLC to give the desired product (Compound 118; 30 mg, 11.11% yield) as a yellow solid. MS (ESI): mass calc'd for C25H36N3OS 426, m/z found 426 [M+H]⁺. ¹H NMR (400 MHz, MeOD) δ 7.68-7.63 (m, 2H), 7.37 (s, 1H), 7.35 (d, J=3.6 Hz, 2H), 4.04-3.91 (m, 2H), 3.88-3.72 (m, 2H), 3.69-3.54 (m, 4H), 3.23 (m, 2H), 2.97 (m, 1H), 2.76 (q, J=7.6 Hz, 2H), 2.44 (m, 1H), 2.22-2.02 (m, 5H), 1.43 (d, J=6.8 Hz, 6H), 1.29 (t, J=7.6 Hz, 3H).

### Example 10: Synthesis of ((S)-3-aminopyrrolidin-1-yl)(3-methyl-5-(4-(1-((tetrahydro-2H-pyran-2-yl)methyl)piperidin-4-yl)phenyl)thiophen-2-yl)methanone (Compound 119)

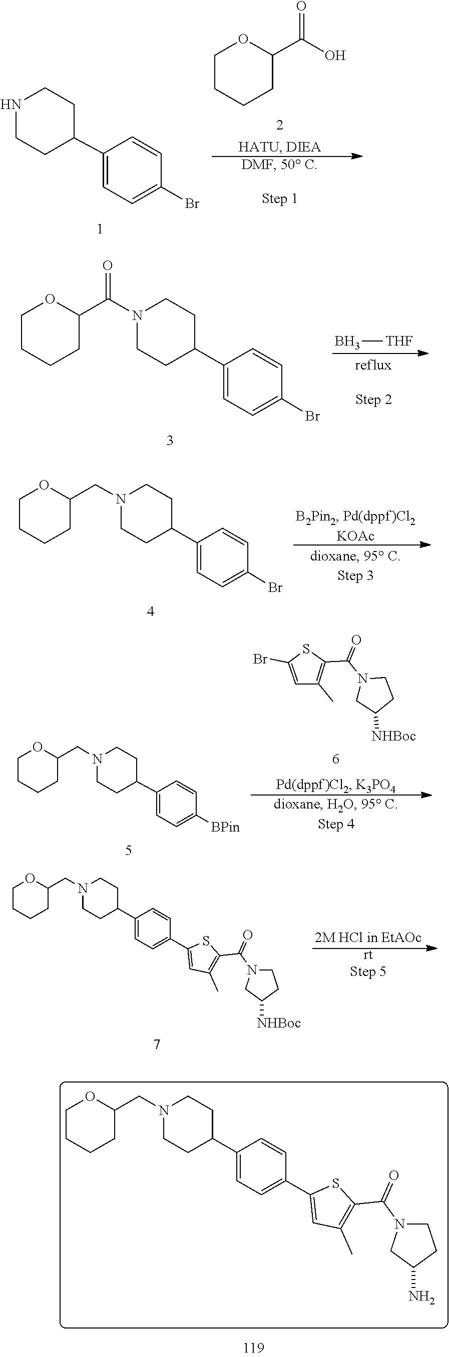

### Step 1 (4-(4-bromophenyl)piperidin-1-yl)(tetrahydro-2H-pyran-2-yl)methanone

To a mixture of 4-(4-bromophenyl)piperidine (600 mg, 2.50 mmol) in DMF (10 mL) were added oxane-2-carboxylic acid (390 mg, 3.00 mmol), DIEA (1.29 g, 9.99 mmol) and HATU (1.14 g, 3.00 mmol), then it was stirred at 50° C. for 12 hours. LCMS showed the reaction was completed. The reaction mixture was diluted with H₂O (30 mL) and extracted with EtOAc (15 mL*3). The combined organic phases were washed with brine (50 mL*2), dried over Na₂SO₄, filtered and concentrated to dryness. The crude product was purified by column chromatography on silica gel (EA/PE=20%) to give the desired product (920 mg, 94.1% yield) as a yellow oil.

### Step 2 4-(4-bromophenyl)-1-((tetrahydro-2H-pyran-2-yl)methyl)piperidine

A mixture of compound 3 (920 mg, 2.6117 mmol) in THF (9 mL) was added BH₃ (144.48 mg, 10.446 mmol), then the mixture was stirred at 75° C. for 4 hours under N₂ atmosphere. LCMS showed the reaction was completed. After the reaction was cooled in an ice bath, methanol (5 mL) was added cautiously. HCl (6 M, 8 mL) was added fast dropwise, and the mixture was heated to reflux for 30 minutes. The volatiles were then concentrated in vacuo, and the resulting mixture was cooled in an ice bath and 50% aqueous NaOH (10 mL) was added (pH>10). The mixture was then diluted with water and extracted with diethyl ether (50 mL*3). The combined organic phases were washed with brine, dried over Na₂SO₄, filtered and concentrated. The crude product was purified by column chromatography on silica gel (EA/PE=20%~30%) to afford compound 4 (830 mg, 86.4% yield) as colorless oil.

### Step 3 1-((tetrahydro-2H-pyran-2-yl)methyl)-4-(4-(4,4,5,5-tetramethyl-1,3,2-dioxaborolan-2-yl)phenyl)piperidine

To a mixture of compound 4 (830 mg, 2.45 mmol) in 1,4-dioxane (15 mL) were added B₂Pin₂ (748 mg, 2.94 mmol), KOAc (722 mg, 7.36 mmol) and Pd(dppf)Cl₂DCM (301 mg, 0.37 mmol), then it was stirred at 100° C. under N₂ for 16 hours. LCMS showed the reaction was completed. The reaction mixture was concentrated to dryness. The crude product was purified by column chromatography on silica gel (DCM/MeOH=10/1) to afford compound 5 (1.00 g, 84.6% yield) as black oil.

### Step 4 tert-butyl ((3S)-1-(3-methyl-5-(4-(1-((tetrahydro-2H-pyran-2-yl)methyl)piperidin-4-yl)phenyl)thiophene-2-carbonyl)pyrrolidin-3-yl)carbamate

To a mixture of compound 5 (500 mg, 1.30 mmol) in 1,4-dioxane (14 mL) and H₂O (2 mL) was added compound 6 (404 mg, 1.04 mmol), K₃PO₄ (826 mg, 3.89 mmol) and Pd(dppf)Cl₂*DCM (159 mg, 0.19 mmol), then it was stirred at 95° C. under N₂ for 16 hours. LCMS showed the reaction was completed. The reaction mixture was concentrated to dryness. The crude product was purified by column chromatography on silica gel (MeOH/DCM=5%) to afford compound 7 (280 mg, 34.2%) as a yellow solid.

### Step 5 ((S)-3-aminopyrrolidin-1-yl)(3-methyl-5-(4-(1-((tetrahydro-2H-pyran-2-yl)methyl)piperidin-4-yl)phenyl)thiophen-2-yl)methanone

A mixture of compound 7 (140 mg, 0.25 mmol) in HCl/EtOAc (2 mL) was stirred at 25° C. for 2 hours. LCMS showed the reaction was completed. The reaction mixture was concentrated to dryness. The crude product was purified by prep-HPLC to afford Compound 119 (109.3 mg, 94.8% yield) as a white solid. LCMS (ESI): mass calc'd. for C₂₇H₃₇N₃O₂S 467.26, m/z found 468.3 [M+H]⁺. ¹H NMR (400 MHz, MeOD) δ 7.61 (d, J=8.4 Hz, 2H), 7.41-7.29 (m, 2H), 7.24 (s, 1H), 4.06-3.95 (m, 3H), 3.83 (s, 3H), 3.75-3.63 (m, 3H), 3.57-3.48 (m, 1H), 3.26-3.03 (m, 4H), 2.98-2.85 (m, 1H), 2.49-2.39 (m, 1H), 2.35 (s, 3H), 2.18-2.05 (m, 4H), 2.03-1.88 (m, 2H), 1.68-1.55 (m, 4H), 1.36-1.26 (m, 1H).

### Example 11: Synthesis of (S)-(3-aminopyrrolidin-1-yl)(5-(2-chloro-4-(1-methylpiperidin-4-yl)phenyl)-3-methylthiophen-2-yl)methanone (Compound 120)

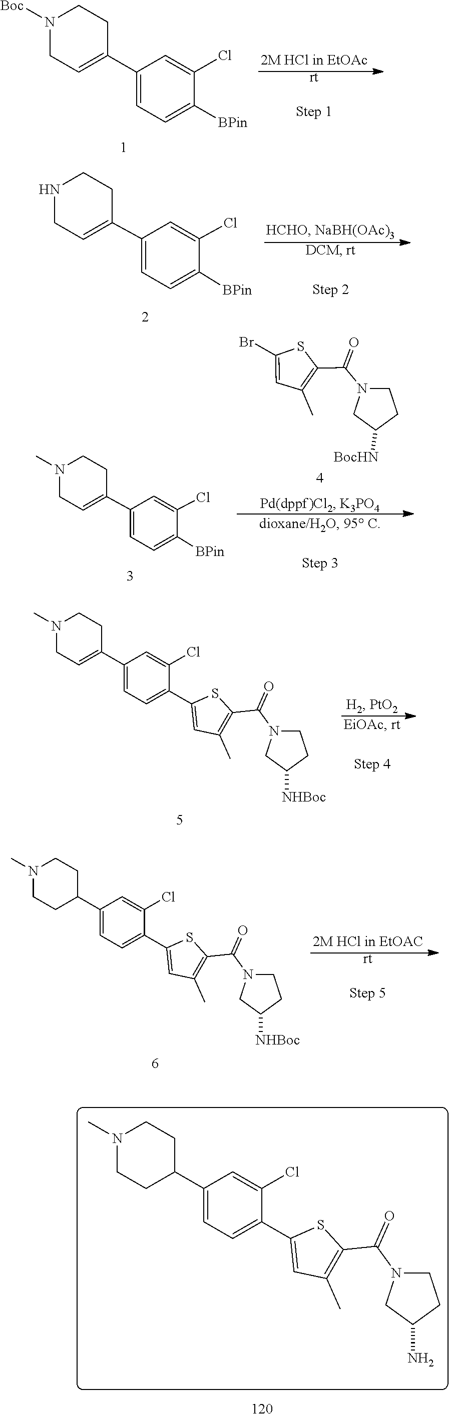

### Step 1 4-(3-chloro-4-(4,4,5,5-tetramethyl-1,3,2-dioxaborolan-2-yl)phenyl)-1, 2, 3,6-tetrahydropyridine

A mixture of tert-butyl 4-(3-chloro-4-(4,4,5,5-tetramethyl-1,3,2-dioxaborolan-2-yl)phenyl)-3,6-dihydropyridine-1(2H)-carboxylate (600 mg, 1.43 mmol) in HCl/EtOAc (6 mL) was stirred at 25° C. for 3 hours. LCMS showed the reaction was completed. The reaction mixture was concentrated to dryness to afford the desired product (560 mg, 86.0% yield) as a yellow solid.

### Step 2 4-(3-chloro-4-(4,4,5,5-tetramethyl-1,3,2-dioxaborolan-2-yl)phenyl)-1-methyl-1,2,3,6-tetrahydropyridine

To a mixture of compound 2 (560 mg, 1.75 mmol) in DCM (50 mL) was added NaBH(OAc)₃ (3.34 g, 15.77 mmol), HCHO (569 mg, 7.01 mmol, 37 wt % in H₂O) and Na₂SO₄ (124 mg, 0.88 mmol), then it was stirred at 25° C. for 12 hours. LCMS showed the reaction was completed. The reaction mixture was concentrated to dryness. The crude product was purified by column chromatography on silica gel (EA/MeOH=5/1) to afford the desired product (480 mg, 73.9% yield) as a yellow solid.

### Step 3 tert-butyl (S)-(1-(5-(2-chloro-4-(1-methyl-1,2,3,6-tetrahydropyridin-4-yl)phenyl)-3-methylthiophene-2-carbonyl)pyrrolidin-3-yl)carbamate

To a mixture of compound 3 (480 mg, 1.44 mmol) in 1,4-dioxane (16 mL) and H₂O (2 mL) were added compound 4 (504 mg, 1.29 mmol), K₃PO₄ (916 mg, 4.32 mmol) and Pd(dppf)Cl₂*DCM (176 mg, 0.22 mmol), then it was stirred at 95° C. under N₂ for 16 hours. LCMS showed the reaction was completed. The reaction mixture was concentrated to dryness. The crude product was purified by column chromatography on silica gel (MeOH/DCM=5%-6%) to afford the desired product (410 mg, 49.7% yield) as a yellow solid.

### Step 4 tert-butyl (S)-(1-(5-(2-chloro-4-(1-methylpiperidin-4-yl)phenyl)-3-methylthiophene-2-carbonyl)pyrrolidin-3-yl)carbamate

To a mixture of compound 5 (410 mg, 0.79 mmol) in EtOAc (4 mL) was added PtO₂ (41 mg, 0.18 mmol), then it was stirred at 25° C. for 32 hours under H₂ atmosphere. LCMS showed the reaction was completed. The reaction mixture was filtered and the filtrate was concentrated to dryness to afford the desired product (390 mg, 85.3% yield) as a yellow solid.

### Step 5 (S)-(3-aminopyrrolidin-1-yl)(5-(2-chloro-4-(1-methylpiperidin-4-yl)phenyl)-3-methylthiophen-2-yl)methanone

A mixture of compound 6 (240 mg, 0.46 mmol) in HCl/EtOAc (3 mL) was stirred at 25° C. for 3 hours. LCMS showed the reaction was completed. The reaction mixture was concentrated to dryness. The crude product was purified by prep-HPLC to afford the desired product (Compound 120; 12.6 mg, 6.2% yield) as a white solid. MS (ESI): mass calc'd. for C₂₂H₂₉ClN₃OS 418.17, m/z found 418.2 [M+H]⁺. ¹H NMR (400 MHz, MeOD) δ 7.58 (d, J=8.4 Hz, 1H), 7.47 (s, 1H), 7.31 (d, J=8.0 Hz, 1H), 7.22 (s, 1H), 4.03-3.96 (m, 2H), 3.89-3.75 (m, 2H), 3.74-3.59 (m, 3H), 3.16 (t, J=12 Hz, 2H), 2.94 (s, 4H), 2.50-2.40 (m, 1H), 2.37 (s, 3H), 2.19-2.10 (m, 3H), 2.07-1.92 (m, 2H).

The following compound was prepared analogously to Compound 120:

| Compound | Name | H NMR | MS |
|---|---|---|---|
| 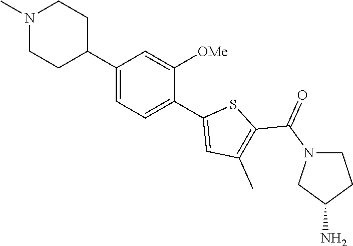 [SMILES: COc1cc(C2CCN(C)CC2)ccc1-c1cc(C)c(C(=O)N2CC[C@H](N)C2)s1] | (S)-(3- aminopyrrolidin-1- yl)(5-(2-methoxy- 4-(1- methylpiperidin-4- yl)phenyl)-3- methylthiophen-2- yl)methanone | ¹H NMR (400 MHz, MeOD) δ 7.56 (d, J = 8.0 Hz, 1H), 7.26 (s, 1H), 6.89 (d, J = 1.3 Hz, 1H), 6.82 (dd, J = 8.1, 1.5 Hz, 1H), 3.93-3.87 (m, 2H), 3.84 (s, 3H), 3.75 (dt, J = 11.8, 7.5 Hz, 1H), 3.69-3.57 (m, 2H), 3.52 (d, J = 12.4 Hz, 2H), 3.04 (tt, J = 9.7, 4.8 Hz, 2H), 2.85-2.75 (m, 4H), 2.33 (td, J = 13.8, 7.5 Hz, 1H), 2.24 (s, 3H), 2.10-1.84 (m, 5H). | 414.00 [M + H]⁺ |
| 113 | | | |

### Example 12: Synthesis of (S)-(3-aminopyrrolidin-1-yl)(3-methyl-5-(2-(1-methylpiperidin-4-yl)pyrimidin-5-yl)thiophen-2-yl)methanone (Compound 124)

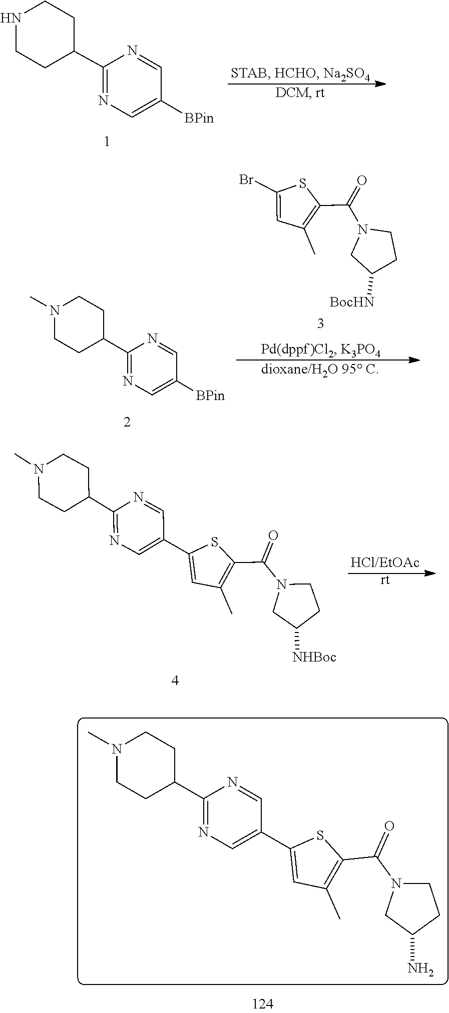

### Step 1 2-(1-methylpiperidin-4-yl)-5-(4,4,5,5-tetramethyl-1,3,2-dioxaborolan-2-yl)pyrimidine

To a mixture of 2-(piperidin-4-yl)-5-(4,4,5,5-tetramethyl-1,3,2-dioxaborolan-2-yl)pyrimidine (100 mg, 0.35 mmol) in DCM (10 mL) was added NaBH(OAc)₃ (660 mg, 3.11 mmol), HCHO (112 mg, 1.38 mmol) and Na₂SO₄ (25 mg, 0.17 mmol), then it was stirred at 25° C. for 12 hours. After the reaction, the mixture was concentrated to dryness. The crude product was purified by column chromatography on silica gel (MeOH/DCM=0%~30%) to give the desired product (200 mg, 85.8% yield) as a white solid.

### Step 2 tert-butyl (S)-(1-(3-methyl-5-(2-(1-methylpiperidin-4-yl)pyrimidin-5-yl)thiophene-2-carbonyl)pyrrolidin-3-yl)carbamate

To a mixture of compound 2 (200 mg, 0.66 mmol) in 1,4-dioxane (8 mL) and H₂O (1 mL) were added compound 3 (231 mg, 0.59 mmol), K₃PO₄ (420 mg, 1.98 mmol) and Pd(dppf)Cl₂*DCM (81 mg, 0.10 mmol), then it was stirred at 95° C. under N₂ for 16 hours. After the reaction, the mixture was concentrated to dryness. The crude product was purified by column chromatography on silica gel using MeOH/DCM=0%~30% as eluent to afford compound 4 (100 mg, 28.1% yield) as a black solid.

### Step 3 (S)-(3-aminopyrrolidin-1-yl)(3-methyl-5-(2-(1-methylpiperidin-4-yl)pyrimidin-5-yl)thiophen-2-yl)methanone

A mixture of compound 4 (100 mg, 0.2059 mmol) in HCl/EtOAc (2 mL) was stirred at 25° C. for 3 hours. The LCMS showed the reaction was completed. The mixture was concentrated to dryness and the crude product was purified by prep-HPLC to afford the desired product Compound 124 (7 mg, 8.7% yield) as a white solid. MS (ESI): mass calc'd. for C20H27N5OS 385.19, m/z found 386.1 [M+H]⁺. ¹H NMR (400 MHz, MeOD) δ 9.01 (s, 2H), 8.52 (s, 2H), 7.45 (s, 1H), 3.95-3.77 (m, 3H), 3.75-3.67 (m, 1H), 3.57-3.41 (m, 3H), 3.22-3.13 (m, 1H), 3.05-2.92 (m, 2H), 2.81-2.75 (m, 3H), 2.37 (s, 3H), 2.35-2.24 (m, 3H), 2.22-2.11 (m, 2H), 2.05-1.97 (m, 1H).

The following compounds are prepared analogously to the methods described in this example:

| Compound | Synthesis |
|---|---|
| 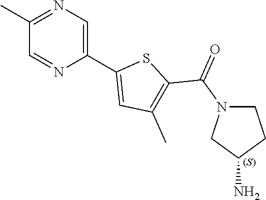 | For step 2, a corresponding boronic ester can be used to synthesize compound 223 |
| 223 | |

### Example 13: Synthesis of (S)-(3-aminopyrrolidin-1-yl)(5-(2-fluoro-4-(1-methylpiperidin-4-yl)phenyl)-3-methylthiophen-2-yl)methanone (Compound 128)

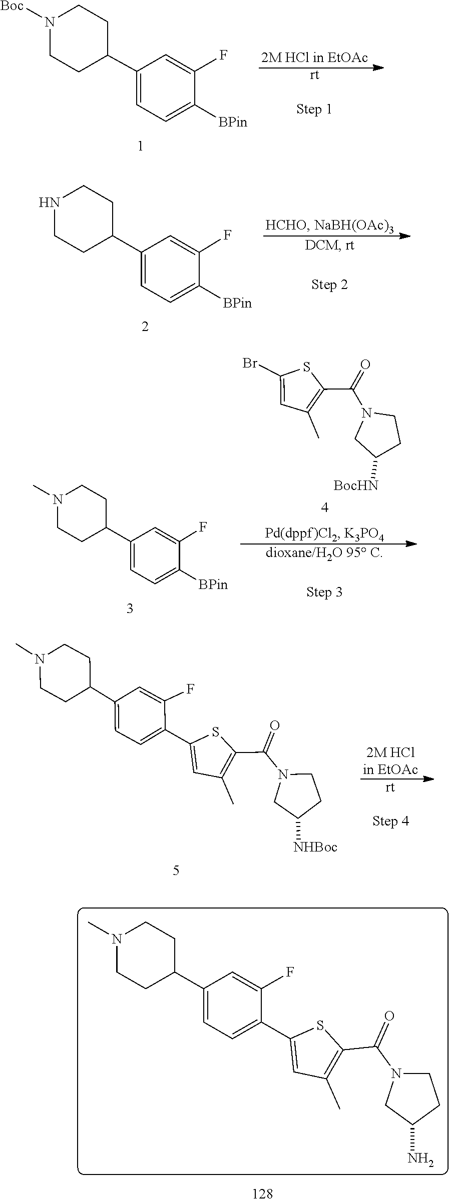

### Step 1 4-(3-fluoro-4-(4,4,5,5-tetramethyl-1,3,2-dioxaborolan-2-yl)phenyl)piperidine

To the solution of tert-butyl 4-(3-fluoro-4-(4,4,5,5-tetramethyl-1,3,2-dioxaborolan-2-yl)phenyl)piperidine-1-carboxylate (600 mg, 1.4767 mmol) in EtOAc (2 mL) was added HCl (5 mL, 2M in EtOAc). The mixture was stirred at rt for 16 h. The LCMS showed the reaction was completed and the desired MS was found. The solvent was removed under reduced pressure to afford the desired product (500 mg, 94.30% yield) as a colorless oil.

### Step 2 4-(3-fluoro-4-(4,4,5,5-tetramethyl-1,3,2-dioxaborolan-2-yl)phenyl)-1-methylpiperidine

To a mixture of 4-(3-fluoro-4-(4,4,5,5-tetramethyl-1,3,2-dioxaborolan-2-yl)phenyl)piperidine (500 mg, 1.6383 mmol) in DCM (50 mL) was added NaBH(OAc)₃ (3124.9 mg, 14.744 mmol), formaldehyde aqueous solution (531.3 mg, 6.5532 mmol) and Na₂SO₄ (69.8 mg, 0.4914 mmol), then it was stirred at 25° C. for 16 hours. The LCMS showed the reaction was completed and the desired MS was found. After the reaction, the solvent was removed under reduced pressure and water was added, the mixture was extracted with EtOAc for 3 times. Combined with EtOAc phases, washed with brine, dried over Na₂SO₄ and filtered. The solvent was removed under reduced pressure to afford the desired product (730 mg, 97.71% yield) as a colorless oil.

### Step 3 tert-butyl(S)-(1-(5-(2-fluoro-4-(1-methylpiperidin-4-yl)phenyl)-3-methylthiophene-2-carbonyl)pyrrolidin-3-yl)carbamate

To the solution of 4-(3-fluoro-4-(4,4,5,5-tetramethyl-1,3,2-dioxaborolan-2-yl)phenyl)-1-methylpiperidine (250 mg, 0.7832 mmol) in dioxane/H₂O=8:1 (9 mL) was added tert-butyl (S)-(1-(5-bromo-3-methylthiophene-2-carbonyl)pyrrolidin-3-yl)carbamate (304.9 mg, 0.7832 mmol), K₃PO₄ (498.7 mg, 2.3496 mmol) and Pd(dppf)Cl₂DCM (127.9 mg, 0.1566 mmol). The mixture was stirred at 95° C. under N₂ for 16 h. The LCMS showed the reaction was completed and the desired MS was found. After cooled to rt, the mixture was filtered and concentrated under reduced pressure. The residue was purified by silica gel column chromatography to give the desired product (150 mg, 34.36% yield) as a yellow solid.

### Step 4 (S)-(3-aminopyrrolidin-1-yl)(5-(2-fluoro-4-(1-methylpiperidin-4-yl)phenyl)-3-methylthiophen-2-yl)methanone

To the solution of tert-butyl(S)-(1-(5-(2-fluoro-4-(1-methylpiperidin-4-yl)phenyl)-3-methylthiophene-2-carbonyl)pyrrolidin-3-yl)carbamate (150 mg, 0.2990 mmol) in EtOAc (2 mL) was added HCl (3 mL, 2M in EtOAc). The mixture was stirred at rt for 16 h. The LCMS showed the reaction was completed and the desired MS was found. The solvent was removed under reduced pressure and the residue was purified by prep-HPLC to give the desired product (Compound 128; 30 mg, 22.47% yield) as a yellow solid. MS (ESI): mass calc'd for C22H29FN3OS 402, m/z found 402 [M+H]+. ¹H NMR (400 MHz, MeOD) δ 7.53 (t, J=8.4 Hz, 1H), 7.19 (s, 1H), 7.02 (m, 2H), 3.66 (m, 2H), 3.51 (m, 2H), 3.23 (s, 1H), 2.91 (d, J=11.6 Hz, 2H), 2.49 (m, 1H), 2.23 (d, J=2.4 Hz, 6H), 2.07 (m, 3H), 1.80-1.62 (m, 5H).

### Example 14: Synthesis of (S)-(3-aminopyrrolidin-1-yl)(5-(2-fluoro-4-(piperidin-4-yl)phenyl)-3-methylthiophen-2-yl)methanone (Compound 129)

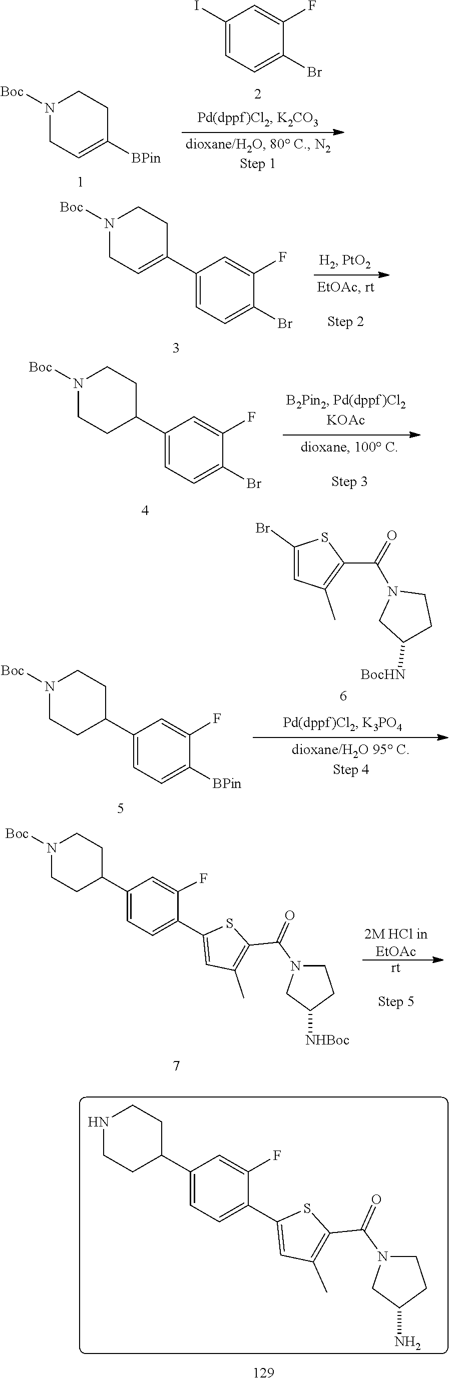

### Step 1 tert-butyl 4-(4-bromo-3-fluorophenyl)-3,6-dihydropyridine-1(2H)-carboxylate

To a mixture of tert-butyl 4-(4,4,5,5-tetramethyl-1,3,2-dioxaborolan-2-yl)-3,6-dihydropyridine-1(2H)-carboxylate (1.50 g, 4.80 mmol) in 1,4-dioxane (16 mL) and H₂O (4 mL) were added 1-bromo-2-fluoro-4-iodobenzene (2.17 g, 7.20 mmol), K₃PO₄ (1.66 g, 12.00 mmol) and Pd(dppf)Cl₂DCM (0.59 g, 0.70 mmol), the mixture was stirred at 80° C. for under N₂ 16 hours. TLC (PE:EA=10:1) showed the reaction was completed. The reaction mixture was concentrated to dryness. The crude product was purified by column chromatography on silica gel (EA/PE=8%) to give the desired product (1.30 g, 68.7% yield) as light-yellow oil.

### Step 2 tert-butyl 4-(4-bromo-3-fluorophenyl)piperidine-1-carboxylate

To a mixture of compound 3 (1.30 g, 3.60 mmol) in EtOAc (30 mL) was added PtO₂ (0.13 g, 0.50 mmol), then it was stirred at 25° C. under H₂ atmosphere for 48 hours. LCMS showed the reaction was completed. The reaction mixture was filtered and the filtrate was concentrated to dryness to afford the desired product (1.30 g, 69.4% yield) as yellow oil.

### Step 3 tert-butyl 4-(3-fluoro-4-(4,4,5,5-tetramethyl-1,3,2-dioxaborolan-2-yl)phenyl)piperidine-1-carboxylate

To a mixture of compound 4 (1.30 g, 3.60 mmol) in 1,4-dioxane (20 mL) were added B₂Pin₂ (1.10 g, 4.30 mmol), KOAc (1.06 g, 10.80 mmol) and Pd(dppf)Cl₂ (0.40 g, 0.50 mmol), then it was stirred at 95° C. under N₂ for 16 hours. LCMS showed the reaction was completed. The reaction mixture was concentrated to dryness. The crude product was purified by column chromatography on silica gel (EA/PE=8%) to afford the desired product (1.10 g, 66.6% yield) as colorless oil.

### Step 4 tert-butyl (S)-4-(4-(5-(3-((tert-butoxycarbonyl)amino)pyrrolidine-1-carbonyl)-4-methylthiophen-2-yl)-3-fluorophenyl)piperidine-1-carboxylate

To a mixture of compound 5 (200 mg, 0.49 mmol) and compound 6 (192 mg, 0.49 mmol) in 1,4-dioxane (8 mL) and H₂O (1 mL) were added K₃PO₄ (313 mg, 1.48 mmol) and Pd(dppf)Cl₂ (60 mg, 0.07 mmol), then it was stirred at 95° C. under N₂ atmosphere for 16 hours. LCMS showed the reaction was completed. The reaction mixture was concentrated to dryness. The crude product was purified by column chromatography on silica gel (EA/PE=30%) to afford the desired product (150 mg, 46.5% yield) was obtained as light-yellow oil. LCMS (ESI): mass calc'd. for C₃₁H₄₃FN₃O₅S 588.29, m/z found 588.5 [M+H]⁺.

### Step 5 (S)-(3-aminopyrrolidin-1-yl)(5-(2-fluoro-4-(piperidin-4-yl)phenyl)-3-methylthiophen-2-yl)methanone

To a mixture of compound 7 (150 mg, 0.25 mmol) in EtOAc (2 mL) was added HCl (5 mL, 2M in EtOAc). The mixture was stirred at 25° C. for 16 h. LCMS showed the reaction was completed. The reaction mixture was concentrated to dryness. The crude product was purified by prep-HPLC to afford the desired product (Compound 129; 10.8 mg, 25.5% yield) was obtained as a white solid. LCMS (ESI): mass calc'd. for C21H26FN3OS 387.18, m/z found 388.3 [M+H]⁺. ¹H NMR (400 MHz, MeOD) δ 8.49 (s, 1H), 7.70 (t, J=8.0 Hz, 1H), 7.35 (s, 1H), 7.24-7.12 (m, 2H), 4.01-3.92 (m, 2H), 3.86-3.72 (m, 2H), 3.68-3.61 (m, 1H), 3.55-3.48 (m, 2H), 3.20-3.09 (m, 2H), 3.02-2.93 (m, 1H), 2.46-2.34 (m, 4H), 2.15-2.03 (m, 3H), 1.98-1.85 (m, 2H).

### Example 15: Synthesis of (S)-(3-aminopyrrolidin-1-yl)(3-methyl-5-(2-methyl-4-(1-methylpiperidin-4-yl)phenyl)thiophen-2-yl)methanone (Compound 134)

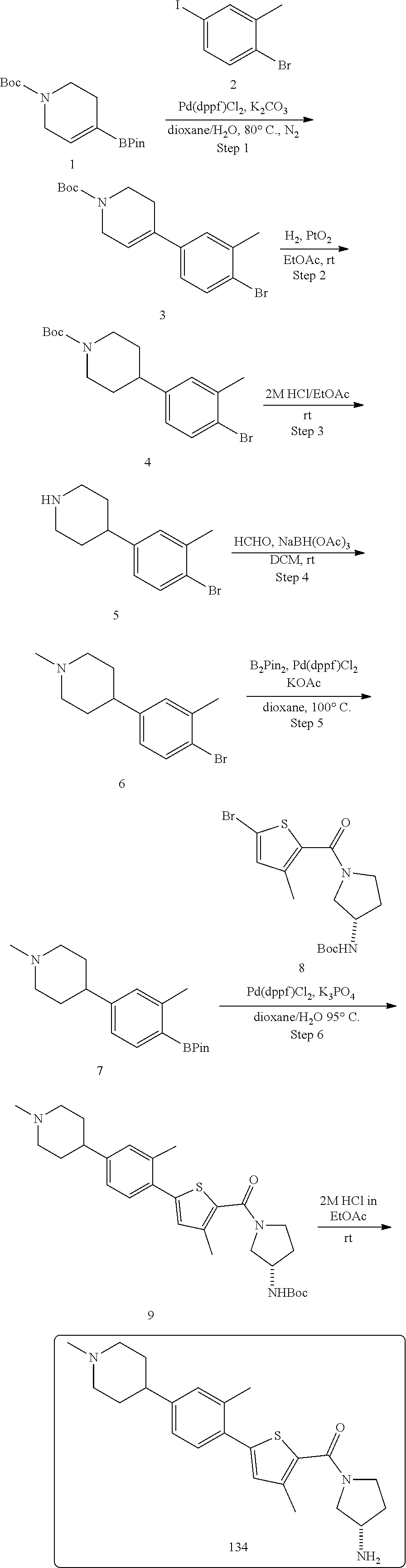

### Step 1 tert-butyl 4-(4-bromo-3-methylphenyl)-3,6-dihydropyridine-1(2H)-carboxylate

To a solution of 1-bromo-4-iodo-2-methylbenzene (1000 mg, 3.4 mmol) in dioxane/H₂O=8:1 (18 mL) were added tert-butyl 4-(4,4,5,5-tetramethyl-1,3,2-dioxaborolan-2-yl)-3,6-dihydropyridine-1(2H)-carboxylate (1044.8 mg, 3.4 mmol), K₂CO₃ (1396.4 mg, 10.1 mmol) and Pd(dppf)Cl₂DCM (549.6 mg, 0.67 mmol). The mixture was stirred at 95° C. under N₂ for 16 h. After cooled to rt, the mixture was filtered and concentrated under reduced pressure. The residue was purified by flash chromatography (petroleum ether/EtOAc=5:1) to give tert-butyl 4-(4-bromo-3-methylphenyl)-3,6-dihydropyridine-1(2H)-carboxylate (580 mg, 46.3% yield) as a yellow oil. LCMS (ESI) calc'd for C17H23BrNO2+ [M+H]+ m/z 352.09, found 352.

### Step 2 tert-butyl 4-(4-bromo-3-methylphenyl)piperidine-1-carboxylate

To a solution of 4-(4-bromo-3-methylphenyl)-1-(4,4,5,5-tetramethyl-1,3,2-dioxaborolan-2-yl)-3,6-dihydro-2H-pyridine (580 mg, 1.53 mmol) in EtOAc (10 mL) was added PtO₂ (34.8 mg, 0.15 mmol). The mixture was stirred at rt under H₂ for 16 h. The LCMS showed the reaction was completed and the desired MS was found. The resulting mixture was filtered and concentrated under reduced pressure to give tert-butyl 4-(4-bromo-3-methylphenyl)piperidine-1-carboxylate (513 mg, 83.6% yield) as a yellow oil. LCMS (ESI) calc'd for C13H19BrN+ [M+H]+ m/z 268.07, found 268.

### Step 3 4-(4-bromo-3-methylphenyl)piperidine

To a solution of tert-butyl 4-(4-bromo-3-methylphenyl)piperidine-1-carboxylate (513 mg, 1.44 mmol) in 5 mL EtOAc was added HCl (5 mL, 2M in EtOAc). The mixture was stirred at rt for 16 h. The LCMS showed the reaction was completed and the desired MS was found. The resulting mixture was concentrated under reduced pressure to give 4-(4-bromo-3-methylphenyl)piperidine (533 mg, 72.62% yield) as a yellow oil. LCMS (ESI) calc'd for C12H17BrN+ [M+H]+ m/z 254.06, found 254.

### Step 4 4-(4-bromo-3-methylphenyl)-1-methylpiperidine

To a solution of 4-(4-bromo-3-methylphenyl)piperidine (533 mg, 2.1 mmol) in DCM (5 mL) was added HCHO (125.8 mg, 4.2 mmol, 37 wt % in H₂O) and NaBH(OAc)₃ (1333.4 mg, 6.3 mmol). The mixture was stirred at rt for 16 h. The LCMS showed the reaction was completed and the desired MS was found. The solvent was removed under reduced pressure and the residue was purified by flash chromatography (petroleum ether/ethyl acetate=1/10) to give 4-(4-bromo-3-methylphenyl)-1-methylpiperidine (200 mg, 33.8% yield) as a yellow oil. LCMS (ESI) calc'd for C13H19BrN+ [M+H]+ m/z 268.07, found 268.

### Step 5 1-methyl-4-(3-methyl-4-(4,4,5,5-tetramethyl-1,3,2-dioxaborolan-2-yl)phenyl)piperidine

To a solution of 4-(4-bromo-3-methylphenyl)-1-methylpiperidine (200 mg, 0.75 mmol) in dioxane (10 mL) were added B₂Pin₂ (189.4 mg, 0.75 mmol), KOAc (220 mg, 2.2 mmol) and Pd(dppf)Cl₂DCM (121.7 mg, 0.15 mmol). The mixture was stirred at 100° C. for 16 h. After cooled to rt, the mixture was filtered and concentrated under reduced pressure. The residue was purified by flash chromatography (EtOAc/petroleum ether=5:1) to give 1-methyl-4-(3-methyl-4-(4,4,5,5-tetramethyl-1,3,2-dioxaborolan-2-yl)phenyl)piperidine (250 mg, 101% yield) as a yellow solid. LCMS (ESI) calc'd for C19H31BNO2+ [M+H]+ m/z 316.25, found 316.

### Step 6 tert-butyl (S)-(1-(3-methyl-5-(2-methyl-4-(1-methylpiperidin-4-yl)phenyl)thiophene-2-carbonyl)pyrrolidin-3-yl)carbamate

To a solution of tert-butyl (S)-(1-(5-bromo-3-methylthiophene-2-carbonyl)pyrrolidin-3-yl)carbamate (280 mg, 0.72 mmol) in dioxane/H₂O=8:1 (18 mL) were added 1-methyl-4-(3-methyl-4-(4,4,5,5-tetramethyl-1,3,2-dioxaborolan-2-yl)phenyl)piperidine (226.7 mg, 0.72 mmol), K₃PO₄ (458 mg, 2.16 mmol) and Pd(dppf)Cl₂DCM (117.4 mg, 0.14 mmol). The mixture was stirred at 95° C. for 16 h. After cooled to rt, the mixture was filtered and concentrated under reduced pressure. The residue was purified by flash chromatography (petroleum ether/EtOAc=1:5) to give tert-butyl (S)-(1-(3-methyl-5-(2-methyl-4-(1-methylpiperidin-4-yl)phenyl)thiophene-2-carbonyl)pyrrolidin-3-yl)carbamate (200 mg, 53% yield) as a yellow oil. LCMS (ESI) calc'd for C28H40N3O3S+ [M+H]+ m/z 498.28, found 498.

### Step 7 (S)-(3-aminopyrrolidin-1-yl)(3-methyl-5-(2-methyl-4-(1-methylpiperidin-4-yl)phenyl)thiophen-2-yl)methanone

A solution of tert-butyl (S)-(1-(3-methyl-5-(2-methyl-4-(1-methylpiperidin-4-yl)phenyl)thiophene-2-carbonyl)pyrrolidin-3-yl)carbamate (200 mg, 0.39 mmol) in 4 mL EtOAc was added HCl (3 mL, 2M in EtOAc). The mixture was stirred at rt for 16 h. The LCMS showed the reaction was completed and the desired MS was found. The mixture was concentrated under reduced pressure and the residue was purified by prep-HPLC to give the (S)-(3-aminopyrrolidin-1-yl)(3-methyl-5-(2-methyl-4-(1-methylpiperidin-4-yl)phenyl)thiophen-2-yl)methanone (Compound 134; 7.1 mg, 4.4% yield) as a yellow solid. LCMS (ESI) calc'd for C24H34N3OS+ [M+H]+ m/z 412.24, found 412. ¹H NMR (400 MHz, MeOD) δ 7.25 (d, J=7.9 Hz, 1H), 7.11 (s, 1H), 7.05 (d, J=7.8 Hz, 1H), 6.84 (s, 1H), 3.88 (t, J=7.2 Hz, 2H), 3.81-3.70 (m, 1H), 3.69-3.57 (m, 2H), 3.52 (d, J=12.1 Hz, 2H), 3.05 (t, J=11.8 Hz, 2H), 2.87-2.73 (m, 4H), 2.40-2.29 (m, 4H), 2.26 (s, 3H), 2.04 (dd, J=17.5, 13.1 Hz, 3H), 1.89 (dd, J=24.7, 12.0 Hz, 2H).

### Example 16: Synthesis of (S)-(3-aminopyrrolidin-1-yl)(5-(3-chloro-4-(1-methylpiperidin-4-yl)phenyl)-3-methylthiophen-2-yl)methanone (Compound 135)

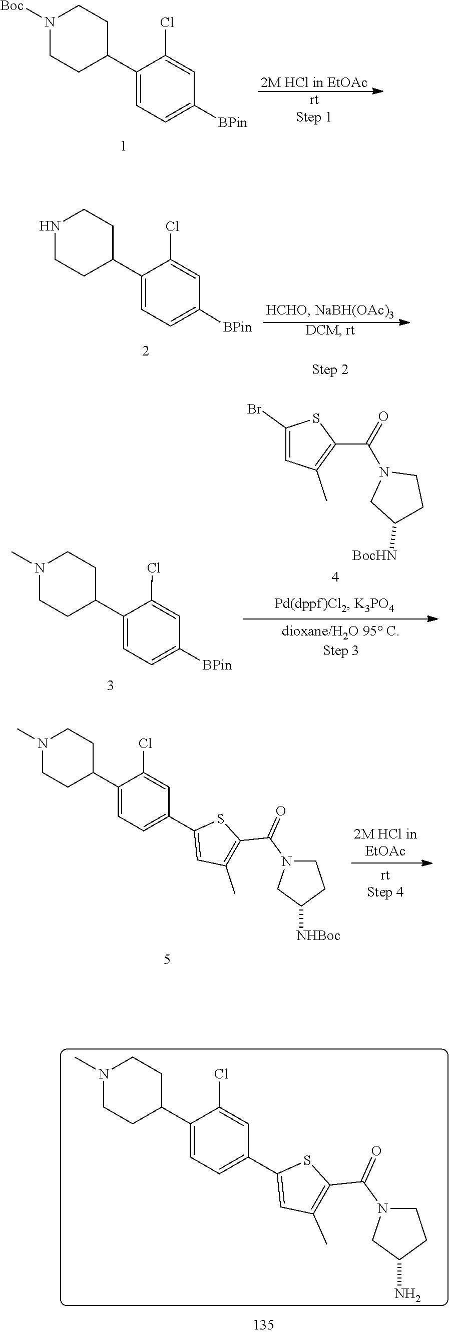

### Step 1 4-(2-chloro-4-(4,4,5,5-tetramethyl-1,3,2-dioxaborolan-2-yl)phenyl)piperidine

A solution of tert-butyl 4-(2-chloro-4-(4,4,5,5-tetramethyl-1,3,2-dioxaborolan-2-yl)phenyl)piperidine-1-carboxylate (292 mg, 0.69 mmol) in HCl (3 mL, 2M in EtOAc) was stirred at 25° C. for 16 hours. LCMS showed the reaction was completed. The reaction mixture was concentrated to dryness to afford the desired product (300 mg, 94.5% yield) as a colorless oil.

### Step 2 4-(2-chloro-4-(4,4,5,5-tetramethyl-1,3,2-dioxaborolan-2-yl)phenyl)-1-methylpiperidine

To a mixture of compound 2 (300 mg, 0.93 mmol) in DCM (30 mL) was added STAB (1.78 g, 8.39 mmol), HCHO (303 mg, 3.73 mmol) and Na₂SO₄ (40 mg, 0.28 mmol), then it was stirred at 25° C. for 16 hours. LCMS showed the reaction was completed. The reaction mixture was concentrated to dryness. Then it was diluted with ethyl acetate and filtrated. The filtrate was concentrated to dryness to afford the desired product (360 mg, 97.7% yield) was obtained as yellow oil.

### Step 3 tert-butyl (S)-(1-(5-(3-chloro-4-(1-methylpiperidin-4-yl)phenyl)-3-methylthiophene-2-carbonyl)pyrrolidin-3-yl)carbamate

To a mixture of compound 3 (285 mg, 0.85 mmol) and compound 4 (300 mg, 0.77 mmol) in 1,4-dioxane (8 mL) and H₂O (1 mL) was added K₃PO₄ (540 mg, 2.54 mmol) and Pd(dppf)Cl₂ (104 mg, 0.13 mmol), then it was stirred at 95° C. for 16 hours under N₂ atmosphere. LCMS showed the reaction was complete. The reaction mixture was concentrated to dryness. The crude product was purified by column chromatography on silica gel (MeOH/DCM=20%) to afford the desired product (227 mg, 43.9% yield) as a black solid.

### Step 4 (S)-(3-aminopyrrolidin-1-yl)(5-(3-chloro-4-(1-methylpiperidin-4-yl)phenyl)-3-methylthiophen-2-yl)methanone

A solution of compound 5 (227 mg, 0.44 mmol) in HCl (3 mL, 2M in EtOAc) was stirred at 25° C. for 16 hours. LCMS showed the reaction was completed. The reaction mixture was concentrated to dryness. The crude product was purified by prep-HPLC to afford the desired product (Compound 135; 58.5 mg, 30.7% yield) as an off-white solid. MS (ESI): mass calc'd. for C22H29ClN₃OS 418.17, m/z found 418.1 [M+H]⁺. ¹H NMR (400 MHz, MeOD) δ 7.65 (d, J=2.0 Hz, 1H), 7.56 (dd, J=8.0, 1.6 Hz, 1H), 7.39 (d, J=8.4 Hz, 1H), 7.26 (s, 1H), 3.82-3.72 (m, 2H), 3.68-3.54 (m, 2H), 3.32 (s, 1H), 3.10-3.00 (m, 3H), 2.35 (s, 3H), 2.33 (s, 3H), 2.26-2.15 (m, 3H), 1.92-1.82 (m, 3H), 1.81-1.72 (m, 2H).

### Example 17: Synthesis of (S)-(3-aminopyrrolidin-1-yl)(5-(3-fluoro-4-(1-methylpiperidin-4-yl)phenyl)-3-methylthiophen-2-yl)methanone (Compound 136)

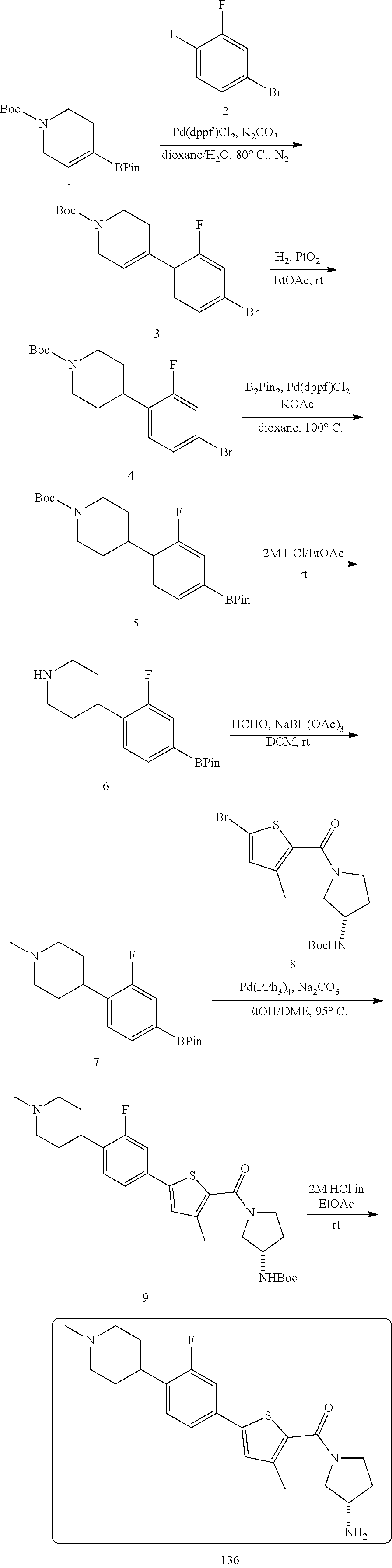

### Step 1 Synthesis of tert-butyl 4-(4-bromo-2-fluorophenyl)-3,6-dihydropyridine-1(2H)-carboxylate

To a solution of tert-butyl 4-(4,4,5,5-tetramethyl-1,3,2-dioxaborolan-2-yl)-3,6-dihydropyridine-1(2H)-carboxylate (1 g, 3.2 mmol) and 4-bromo-2-fluoro-1-iodobenzene (1.44 g, 4.8 mmol) in dioxane/H₂O=4:1 (10 mL) were added K₂CO₃ (1.33 g, 9.6 mmol) and Pd(dppf)Cl₂DCM (0.26 g, 0.36 mmol), the mixture was stirred at 80° C. under N₂ for 16 h. After the reaction, the solvent was removed under reduced pressure, the residue was purified by column chromatography to afford tert-butyl 4-(4-bromo-2-fluorophenyl)-3,6-dihydropyridine-1(2H)-carboxylate (0.84 g, 71.88% yield) as a colorless oil. ¹H NMR (400 MHz, CDCl3) δ 7.26-7.21 (m, 2H), 7.13-7.09 (m, 1H), 5.94-5.92 (m, 1H), 4.06 (9, J=5.8, 2H), 3.61 (t, J=5.8 Hz, 2H), 2.49-2.44 (m, 2H), 1.49 (s, 9H).

### Step 2 Synthesis of tert-butyl 4-(4-bromo-2-fluorophenyl)piperidine-1-carboxylate

To a solution of tert-butyl 4-(4-bromo-2-fluorophenyl)-3,6-dihydropyridine-1(2H)-carboxylate (840 mg, 2.3514 mmol) in 5 mL EtOAc was added PtO₂ (84.4 mg, 0.3715 mmol), the mixture was stirred under H₂ atmosphere at 25° C. for 48 h. After the reaction, the solvent was removed under reduced pressure, the residue was purified by column chromatography to afford tert-butyl 4-(4-bromo-2-fluorophenyl)piperidine-1-carboxylate (500 mg, 58.6% yield) as a colorless oil. LCMS (ESI): calc'd. for C15H21BrFNO2+ [M-Me+H] m/z 345.0, found 344.9.

### Step 3 Synthesis of tert-butyl 4-(2-fluoro-4-(4,4,5,5-tetramethyl-1,3,2-dioxaborolan-2-yl)phenyl)piperidine-1-carboxylate

To a solution of tert-butyl 4-(4-bromo-2-fluorophenyl)piperidine-1-carboxylate (500 mg, 1.3918 mmol) and B₂Pin₂ (424.1 mg, 1.6701 mmol) in dioxane (10 mL) were added Pd(dppf)Cl₂DCM (227.3 mg, 0.2783 mmol) and KOAc (409.8 mg, 4.1754 mmol), the mixture was stirred at 95° C. under N₂ for 16 h. After the reaction, the solvent was removed under reduced pressure, the residue was purified by column chromatography to afford tert-butyl 4-(2-fluoro-4-(4,4,5,5-tetramethyl-1,3,2-dioxaborolan-2-yl)phenyl)piperidine-1-carboxylate (438 mg, 75.9% yield) as a white solid. LCMS (ESI): calc'd. for C21H31BFNO4 [M-Me] m/z 391.23, found 391.3.

### Step 4 Synthesis of 4-(2-fluoro-4-(4,4,5,5-tetramethyl-1,3,2-dioxaborolan-2-yl)phenyl)piperidine

To a solution of tert-butyl 4-(2-fluoro-4-(4,4,5,5-tetramethyl-1,3,2-dioxaborolan-2-yl)phenyl)piperidine-1-carboxylate (438 mg, 1.078 mmol) in 2 mL EtOAc was added HCl (4 mL, 2M in EtOAc), the mixture was stirred at 25° C. for 12 hours. After the reaction, the solvent was removed under reduced pressure to afford 4-(2-fluoro-4-(4,4,5,5-tetramethyl-1,3,2-dioxaborolan-2-yl)phenyl)piperidine (400 mg, 97.26% yield) as a yellowish solid. LCMS (ESI) calc'd for C17H26BFNO2+ [M+H] m/z 306.20, found 306.3.

### Step 5 Synthesis of 4-(2-fluoro-4-(4,4,5,5-tetramethyl-1,3,2-dioxaborolan-2-yl)phenyl)-1-methylpiperidine

To a solution of 4-(2-fluoro-4-(4,4,5,5-tetramethyl-1,3,2-dioxaborolan-2-yl)phenyl)piperidine (400 mg, 1.3106 mmol) in DCM were added HCHO (157.43 mg, 5.2424 mmol) and NaBH(OAc)₃ (833.3 mg, 3.9318 mmol), the mixture was stirred at 25° C. for 2 hours. After the reaction, the mixture was filtered and the solvent was removed under reduced pressure to afford 4-(2-fluoro-4-(4,4,5,5-tetramethyl-1,3,2-dioxaborolan-2-yl)phenyl)-1-methylpiperidine (400 mg, 90.83% yield) as a yellowish oil. LCMS (ESI) calc'd for C18H28BFNO2+ [M+H] m/z 320.21, found 320.1.

### Step 6 Synthesis of tert-butyl (S)-(1-(5-(3-fluoro-4-(1-methylpiperidin-4-yl)phenyl)-3-methylthiophene-2-carbonyl)pyrrolidin-3-yl)carbamate

To a solution of tert-butyl (S)-(1-(5-bromo-3-methylthiophene-2-carbonyl)pyrrolidin-3-yl)carbamate (150 mg, 0.3853 mmol) and 4-(2-fluoro-4-(4,4,5,5-tetramethyl-1,3,2-dioxaborolan-2-yl)phenyl)-1-methylpiperidine (160 mg, 0.5008 mmol) in EtOH/DME (1:1) were added Na₂CO₃ (122.5 mg, 1.1559 mmol) and Pd(PPh₃)₄ (44.5 mg, 0.0385 mmol), the mixture was stirred at 95° C. under N₂ for 16 hours. After the reaction, the solvent was removed under reduced pressure and the residue was purified by column chromatography to afford tert-butyl (S)-(1-(5-(3-fluoro-4-(1-methylpiperidin-4-yl)phenyl)-3-methylthiophene-2-carbonyl)pyrrolidin-3-yl)carbamate (60 mg, 30.42% yield) as a white solid. LCMS (ESI) calc'd for C27H37FN3O3S+ [M+H] m/z 502.25, found 502.3.

### Step 7 Synthesis of (S)-(3-aminopyrrolidin-1-yl)(5-(3-fluoro-4-(1-methylpiperidin-4-yl)phenyl)-3-methylthiophen-2-yl)methanone

To a solution of tert-butyl (S)-(1-(5-(3-fluoro-4-(1-methylpiperidin-4-yl)phenyl)-3-methylthiophene-2-carbonyl)pyrrolidin-3-yl)carbamate (60 mg, 0.1196 mmol) in 2 mL EtOAc was added HCl (4 mL, 2M in EtOAc), the mixture was stirred at 25° C. for 4 hours. After the reaction, the mixture was filtered and the filter cake was washed with EtOAc for several times to afford (S)-(3-aminopyrrolidin-1-yl)(5-(3-fluoro-4-(1-methylpiperidin-4-yl)phenyl)-3-methylthiophen-2-yl)methanone (Compound 136; 13.7 mg, 27.34% yield) as a white solid.

LCMS (ESI) calc'd for C22H29FN3OS+ [M+H] m/z 402.20, found 402.2. ¹H NMR (400 MHz, MeOD) δ 7.47 (d, J=8.0 Hz, 1H), 7.42-7.36 (m, 2H), 7.30 (s, 1H), 4.01-3.97 (m, 2H), 3.88-3.70 (m, 3H), 3.64 (d, J=12.1 Hz, 2H), 3.25-3.20 (m, 3H), 2.93 (s, 3H), 2.48-2.43 (m, 1H), 2.36 (s, 3H), 2.18-2.10 (m, 5H).

### Example 18: Synthesis of ((S)-3-aminopyrrolidin-1-yl)(3-methyl-5-(4-(1-((tetrahydro-2H-pyran-3-yl)methyl)piperidin-4-yl)phenyl)thiophen-2-yl)methanone (Compound 139)

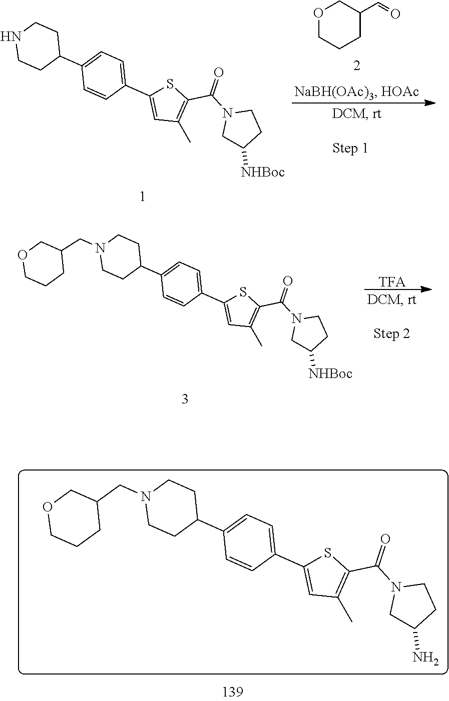 [SMILES: Cc1cc(-c2ccc(C3CCNCC3)cc2)sc1C(=O)N1CC[C@H](NC(=O)OC(C)(C)C)C1 ; O=CC1CCCOC1 ; Cc1cc(-c2ccc(C3CCN(CC4CCCOC4)CC3)cc2)sc1C(=O)N1CC[C@H](NC(=O)OC(C)(C)C)C1 ; Cc1cc(-c2ccc(C3CCN(CC4CCCOC4)CC3)cc2)sc1C(=O)N1CC[C@H](N)C1]

### Step 1 tert-butyl((3S)-1-(3-methyl-5-(4-(1-((tetrahydro-2H-pyran-3-yl)methyl)piperidin-4-yl)phenyl)thiophene-2-carbonyl)pyrrolidin-3-yl)carbamate

To the solution of tert-butyl (S)-(1-(3-methyl-5-(4-(piperidin-4-yl)phenyl)thiophene-2-carbonyl)pyrrolidin-3-yl)carbamate (340 mg, 0.724 mmol) in DCM (6 mL) were added oxane-3-carbaldehyde (247.9 mg, 2.172 mmol), NaBH(OAc)₃ (230.17 mg, 1.086 mmol) and HOAc (87 mg, 1.448 mmol). The mixture was stirred at rt for 16 h. The LCMS showed the reaction was completed and the desired MS was found. The resulting mixture was diluted with H₂O (20 mL) and extracted with EtOAc for 3 times. The combined organic layer was washed with brine, dried over Na₂SO₄, filtered and concentrated under reduced pressure. The residue was purified by silica gel column chromatography to give the desired product (300 mg, 65.68% yield) as a yellow oil.

### Step 2 ((S)-3-aminopyrrolidin-1-yl)(3-methyl-5-(4-(1-((tetrahydro-2H-pyran-3-yl)methyl)piperidin-4-yl)phenyl)thiophen-2-yl)methanone

To the solution of tert-butyl((3S)-1-(3-methyl-5-(4-(1-((tetrahydro-2H-pyran-3-yl)methyl)piperidin-4-yl)phenyl)thiophene-2-carbonyl)pyrrolidin-3-yl)carbamate (300 mg, 0.5284 mmol) in DCM (5 mL) was added TFA (903.72 mg, 7.926 mmol). The mixture was stirred at rt for 16 h. The LCMS showed the reaction was completed and the desired MS was found. The residue was purified by prep-HPLC to give the desired product (Compound 139; 30 mg, 10.92% yield) as a yellow solid. MS (ESI): mass calc'd for C27H38N3O2S 468, m/z found 468 [M+H]⁺. ¹H NMR (400 MHz, MeOD) δ 7.62 (d, J=8.4 Hz, 2H), 7.33 (d, J=8.4 Hz, 2H), 7.25 (s, 1H), 4.01-3.91 (m, 3H), 3.86-3.66 (m, 6H), 3.54-3.47 (m, 1H), 3.19-3.03 (m, 4H), 2.94 (t, J=11.6 Hz, 1H), 2.44 (m, 1H), 2.35 (s, 3H), 2.24-1.92 (m, 8H), 1.68 (m, 2H), 1.51-1.41 (m, 1H).

### Example 19: Synthesis of (S)-(3-aminopyrrolidin-1-yl)(5-(3-fluoro-4-(piperidin-4-yl)phenyl)-3-methylthiophen-2-yl)methanone (Compound 140)

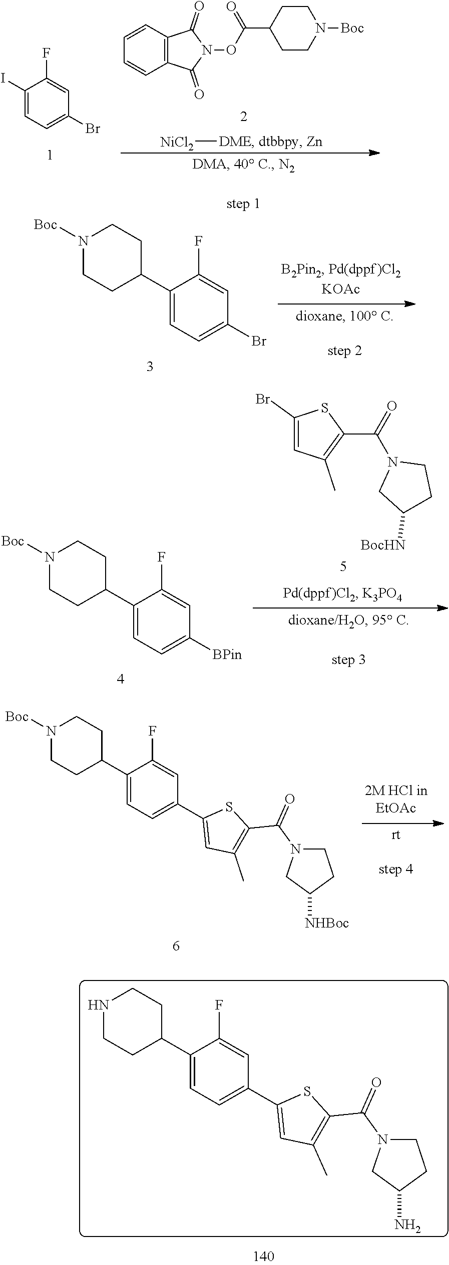

### Step 1 Synthesis of tert-butyl 4-(4-bromo-2-fluorophenyl)piperidine-1-carboxylate

To a solution of NiCl₂·DME (0.07 g, 0.3 mmol) and dtbppy (0.1 g, 0.3 mmol) in 5 mL DMA were added 4-bromo-2-fluoro-1-iodobenzene (0.5 g, 1.7 mmol), 1-(tert-butyl) 4-(1,3-dioxoisoindolin-2-yl) piperidine-1,4-dicarboxylate (0.96 g, 2.5 mmol) and Zn powder (0.22 g, 3.4 mmol), the mixture was stirred at 40° C. under N₂ for 16 hours. After the reaction, H₂O was added and the mixture was extracted with EtOAc for 3 times. Combined with EtOAc phases, washed with brine, dried over Na₂SO₄ and filtered, the solvent was removed under reduced pressure, the residue was purified by column chromatography to afford tert-butyl 4-(4-bromo-2-fluorophenyl)piperidine-1-carboxylate (183 mg, 29.41% yield) as a colorless oil.

### Step 2 Synthesis of tert-butyl 4-(2-fluoro-4-(4,4,5,5-tetramethyl-1,3,2-dioxaborolan-2-yl)phenyl)piperidine-1-carboxylate

To a solution of tert-butyl 4-(4-bromo-2-fluorophenyl)piperidine-1-carboxylate (183 mg, 0.5094 mmol) and B₂Pin₂ (155.3 mg, 0.6112 mmol) in dioxane (6 mL) were added Pd(dppf)Cl₂DCM (41.6 mg, 0.0509 mmol) and KOAc (150 mg, 1.5282 mmol), the mixture was stirred at 100° C. under N₂ for 16 h. After the reaction, the solvent was removed under reduced pressure, the residue was purified by column chromatography to afford tert-butyl 4-(2-fluoro-4-(4,4,5,5-tetramethyl-1,3,2-dioxaborolan-2-yl)phenyl)piperidine-1-carboxylate (150 mg, 71.02% yield) as a white solid.

### Step 3 Synthesis of tert-butyl (S)-4-(4-(5-(3-((tert-butoxycarbonyl)amino)pyrrolidine-1-carbonyl)-4-methylthiophen-2-yl)-2-fluorophenyl)piperidine-1-carboxylate

To a solution of tert-butyl (S)-(1-(5-bromo-3-methylthiophene-2-carbonyl)pyrrolidin-3-yl)carbamate (140 mg, 0.3596 mmol) and tert-butyl 4-(2-fluoro-4-(4,4,5,5-tetramethyl-1,3,2-dioxaborolan-2-yl)phenyl)piperidine-1-carboxylate (146.1 mg, 0.3596 mmol) in dioxane/H₂O (9 mL, v:v=8:1) were added K₃PO₄ (229 mg, 1.0788 mmol) and Pd(dppf)Cl₂DCM (58.7 mg, 0.0719 mmol), the mixture was stirred at 95° C. under N₂ for 16 hours. After the reaction, the solvent was removed under reduced pressure, the residue was purified by column chromatography to afford tert-butyl (S)-4-(4-(5-(3-((tert-butoxycarbonyl)amino)pyrrolidine-1-carbonyl)-4-methylthiophen-2-yl)-2-fluorophenyl)piperidine-1-carboxylate (90 mg, 41.46% yield) as a white solid. LCMS (ESI): calc'd. for C31H43FN3O5S+ [M+H] m/z 588.29, found 588.3.

### Step 4 Synthesis of (S)-(3-aminopyrrolidin-1-yl)(5-(3-fluoro-4-(piperidin-4-yl)phenyl)-3-methylthiophen-2-yl)methanone

To a solution of tert-butyl (S)-4-(4-(5-(3-((tert-butoxycarbonyl)amino)pyrrolidine-1-carbonyl)-4-methylthiophen-2-yl)-2-fluorophenyl)piperidine-1-carboxylate (85 mg, 0.1444 mmol) in 2 mL EtOAc was added HCl (4 mL, 2M in EtOAc), the mixture was stirred at 25° C. for 12 hours. After the reaction, the solvent was removed under reduced pressure and the residue was purified by prep-HPLC to afford the (S)-(3-aminopyrrolidin-1-yl)(5-(3-fluoro-4-(piperidin-4-yl)phenyl)-3-methylthiophen-2-yl)methanone (Compound 140; 13.7 mg, 24.24% yield) as a yellow solid. LCMS (ESI) calc'd for C21H27FN3OS+ [M+H]m/z 388.19, found 388.1. ¹H NMR (400 MHz, MeOD) δ 7.47-7.45 (m, 1H), 7.42-7.35 (m, 2H), 7.29 (s, 1H), 3.99-3.94 (m, 2H), 3.87-3.80 (m, 1H), 3.78-3.71 (m, 1H), 3.69-3.63 (m, 1H), 3.52 (d, J=12.8 Hz, 2H), 3.27-3.14 (m, 3H), 2.46-2.37 (m, 1H), 2.35 (s, 3H), 2.15-1.97 (m, 5H).

### Example 20: Synthesis of (S)-(3-aminopyrrolidin-1-yl)(5-(4-(1-(3-methoxypropyl)piperidin-4-yl)phenyl)-3-methylthiophen-2-yl)methanone (Compound 141)

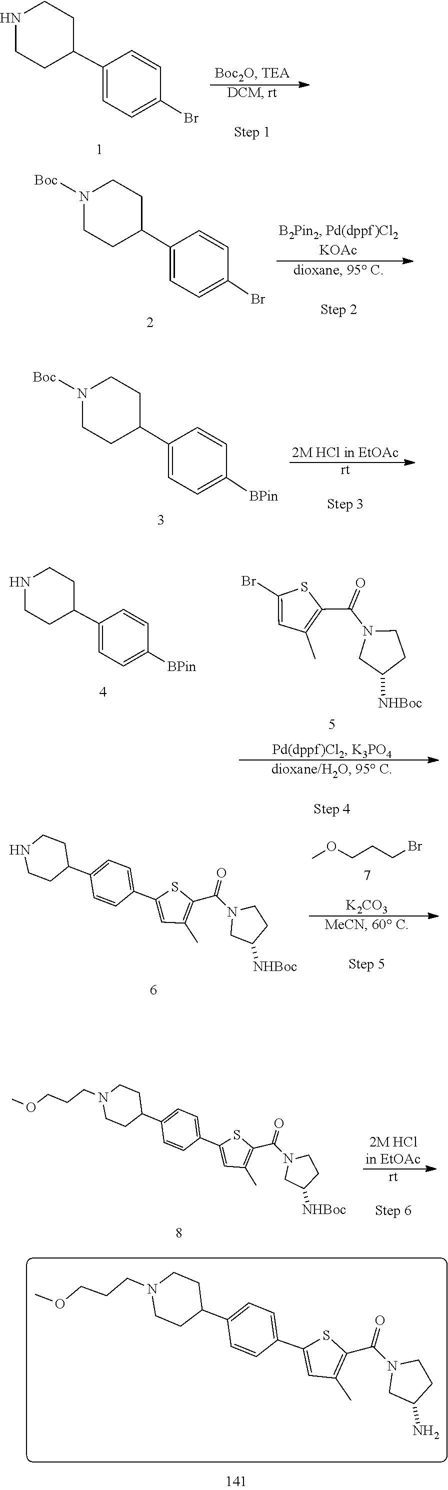

### Step 1 Synthesis of tert-butyl 4-(4-bromophenyl)piperidine-1-carboxylate

To a solution of 4-(4-bromophenyl)piperidine (8 g, 33.3 mmol) and Boc₂O (8.72 g, 39.9 mmol) in 100 mL DCM was added TEA, the mixture was stirred at 25° C. for 12 hours. After the reaction, the solvent was removed under reduced pressure, the residue was purified by column chromatography to afford tert-butyl 4-(4-bromophenyl)piperidine-1-carboxylate (10.09 g, 87.99% yield) as a yellowish oil. ¹H NMR (400 MHz, CDCl3) δ 7.42 (d, J=8.2 Hz, 2H), 7.07 (d, J=8.2 Hz, 2H), 4.24 (d, J=13.2 Hz, 1H), 2.79 (t, J=12.2 Hz, 2H), 2.64-2.57 (m, 1H), 1.81-1.78 (m, 2H), 1.63-1.53 (m, 3H), 1.48 (s, 9H).

### Step 2 Synthesis of tert-butyl 4-(4-(4,4,5,5-tetramethyl-1,3,2-dioxaborolan-2-yl)phenyl)piperidine-1-carboxylate

To a solution of tert-butyl 4-(4-bromophenyl)piperidine-1-carboxylate (10 g, 29.3 mmol), B₂Pin₂ (8.93 g, 35.1 mmol) and KOAc (8.63 g, 87.9 mmol) in 100 mL dioxane was added Pd(dppf)Cl₂DCM (2.39 g, 2.9 mmol), the mixture was stirred at 95° C. under N₂ for 16 hours. After the reaction, the solvent was removed under reduced pressure, the residue was purified by column chromatography to afford tert-butyl 4-(4-(4,4,5,5-tetramethyl-1,3,2-dioxaborolan-2-yl)phenyl)piperidine-1-carboxylate (10 g, 87.03% yield) as a yellow solid. ¹H NMR (400 MHz, CDCl3) δ 7.76 (d, J=7.8 Hz, 2H), 7.22 (d, J=7.8 Hz, 2H), 4.24 (d, J=13.4 Hz, 1H), 2.79 (t, J=12.6, 2H), 2.68-2.62 (m, 1H), 1.81 (d, J=12.6 Hz, 1H), 1.68-1.58 (m, 1H), 1.48 (s, 9H), 1.33 (s, 12H).

### Step 3 Synthesis of 4-(4-(4,4,5,5-tetramethyl-1,3,2-dioxaborolan-2-yl)phenyl)piperidine

To a solution of tert-butyl 4-(4-(4,4,5,5-tetramethyl-1,3,2-dioxaborolan-2-yl)phenyl)piperidine-1-carboxylate (10 g, 25.8 mmol) in 20 mL EtOAc was added HCl (60 mL, 2M in EtOAc), the mixture was stirred at 25° C. for 16 hours. After the reaction, the solvent was removed under reduced pressure to afford 4-(4-(4,4,5,5-tetramethyl-1,3,2-dioxaborolan-2-yl)phenyl)piperidine hydrochloride (8 g, 94.96% yield) as a yellow solid. LCMS (ESI): calc'd. for C17H27BNO2+ [M+H] m/z 288.21, found 288.2.

### Step 4 Synthesis of tert-butyl (S)-(1-(3-methyl-5-(4-(piperidin-4-yl)phenyl)thiophene-2-carbonyl)pyrrolidin-3-yl)carbamate

To a solution of tert-butyl (S)-(1-(5-bromo-3-methylthiophene-2-carbonyl)pyrrolidin-3-yl)carbamate (9.62 g, 24.7 mmol), 4-(4-(4,4,5,5-tetramethyl-1,3,2-dioxaborolan-2-yl)phenyl)piperidine hydrochloride (8 g, 24.7 mmol) and K₃PO₄ (15.73 g, 74.1 mmol) in dioxane/H₂O (90 mL, v:v=8:1) was added Pd(dppf)Cl₂DCM (2.02 g, 2.47 mmol), the mixture was stirred at 95° C. under N₂ for 16 hours. After the reaction, the solvent was removed under reduced pressure, the residue was purified by column chromatography to afford tert-butyl (S)-(1-(3-methyl-5-(4-(piperidin-4-yl)phenyl)thiophene-2-carbonyl)pyrrolidin-3-yl)carbamate (14 g crude, 92.48% yield) as a brown solid. LCMS (ESI) calc'd for C26H36N3O3S+ [M+H] m/z 470.25, found 470.3.

### Step 5 Synthesis of (S)-(3-aminopyrrolidin-1-yl)(5-(4-(1-(3-methoxypropyl)piperidin-4-yl)phenyl)-3-methylthiophen-2-yl)methanone

To a solution of tert-butyl (S)-(1-(3-methyl-5-(4-(piperidin-4-yl)phenyl)thiophene-2-carbonyl)pyrrolidin-3-yl)carbamate (300 mg, 0.6388 mmol) and 1-bromo-3-methoxypropane (195.5 mg, 1.2776 mmol) in 5 mL MeCN was added K₂CO₃, the mixture was stirred at 70° C. for 16 hours. After the reaction, the solvent was removed under reduced pressure, the residue was purified by column chromatography to afford tert-butyl (S)-(1-(5-(4-(1-(3-methoxypropyl)piperidin-4-yl)phenyl)-3-methylthiophene-2-carbonyl)pyrrolidin-3-yl)carbamate (160 mg, 43.93% yield) as a yellow solid. LCMS (ESI) calc'd for C30H44N3O4S+ [M+H] m/z 542.31, found 542.3.

### Step 6 Synthesis of tert-butyl (S)-(1-(5-(4-(1-(3-methoxypropyl)piperidin-4-yl)phenyl)-3-methylthiophene-2-carbonyl)pyrrolidin-3-yl)carbamate

To a solution of tert-butyl (S)-(1-(5-(4-(1-(3-methoxypropyl)piperidin-4-yl)phenyl)-3-methylthiophene-2-carbonyl)pyrrolidin-3-yl)carbamate (160 mg, 0.2953 mmol) in 2 mL EtOAc was added HCl (4 mL, 2M in EtOAc), the mixture was stirred at 25° C. for 12 hours. After the reaction, the mixture was filtered, the filter cake was washed with EtOAc for several times to afford (S)-(3-aminopyrrolidin-1-yl)(5-(4-(1-(3-methoxypropyl)piperidin-4-yl)phenyl)-3-methylthiophen-2-yl)methanone (Compound 141; 15.2 mg, 10.8% yield) as a yellow solid.

LCMS (ESI) calc'd for C25H36N3O2S+ [M+H] m/z 442.25, found 442.2. ¹H NMR (400 MHz, MeOD) δ 7.62 (d, J=8.2 Hz, 2H), 7.34 (d, J=8.2 Hz, 2H), 7.25 (s, 1H), 4.01-3.98 (m, 2H), 3.87-3.68 (m, 5H), 3.54-3.52 (m, 2H), 3.37 (s, 3H), 3.28-3.24 (m, 2H), 3.17-3.11 (m, 2H), 2.98-2.92 (m, 1H), 2.50-2.41 (m, 1H), 2.36 (s, 3H), 2.16-2.00 (m, 7H).

The following compounds are prepared analogously to the methods described in this example:

| Compound | Synthetic procedure |
|---|---|
| 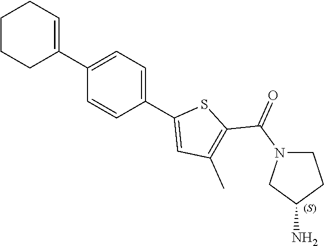 | For step 2, a corresponding aryl bromide can be used to synthesize compound 224 |
| 224 | |
| 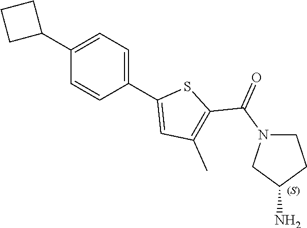 | For step 2, a corresponding aryl bromide can be used to synthesize compound 232 |
| 232 | |
| 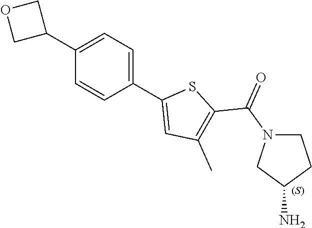 | For step 2, a corresponding aryl bromide can be used to synthesize compound 235 |
| 235 | |
| 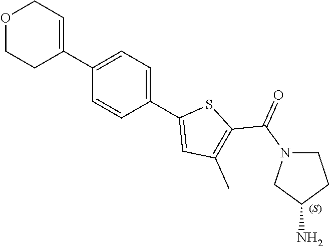 | For step 2, a corresponding aryl bromide can be used to synthesize compound 236 |
| 236 | |
| 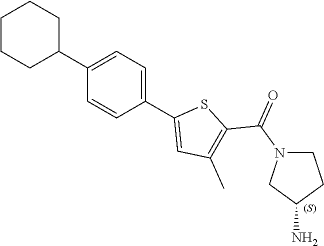 | For step 2, a corresponding aryl bromide can be used to synthesize compound 238 |
| 238 | |
| 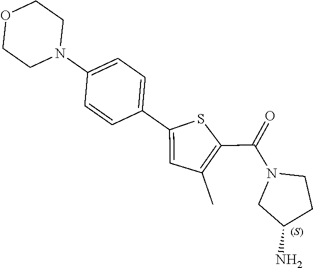 | For step 2, a corresponding aryl bromide can be used to synthesize compound 239 |
| 239 | |
| 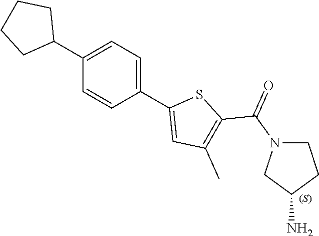 | For step 2, a corresponding aryl bromide can be used to synthesize compound 241 |
| 241 | |
| 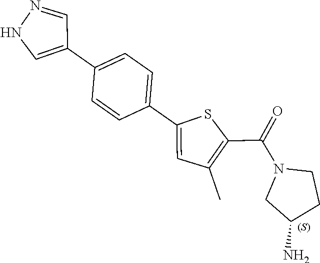 | For step 2, a corresponding aryl bromide can be used to synthesize compound 242 |
| 242 | |
| 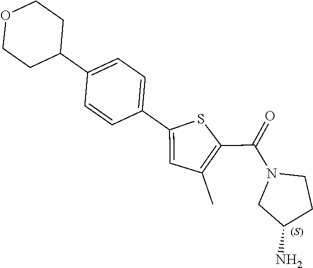 | For step 2, a corresponding aryl bromide can be used to synthesize compound 243 |
| 243 | |
| 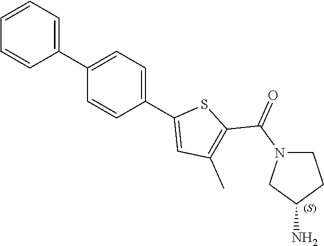 | For step 2, a corresponding aryl bromide can be used to synthesize compound 234 |
| 234 | |

### Example 21: Synthesis of (S)-(3-aminopyrrolidin-1-yl)(5-(4-(1-(2-methoxyethyl)piperidin-4-yl)phenyl)-3-methylthiophen-2-yl)methanone (Compound 142)

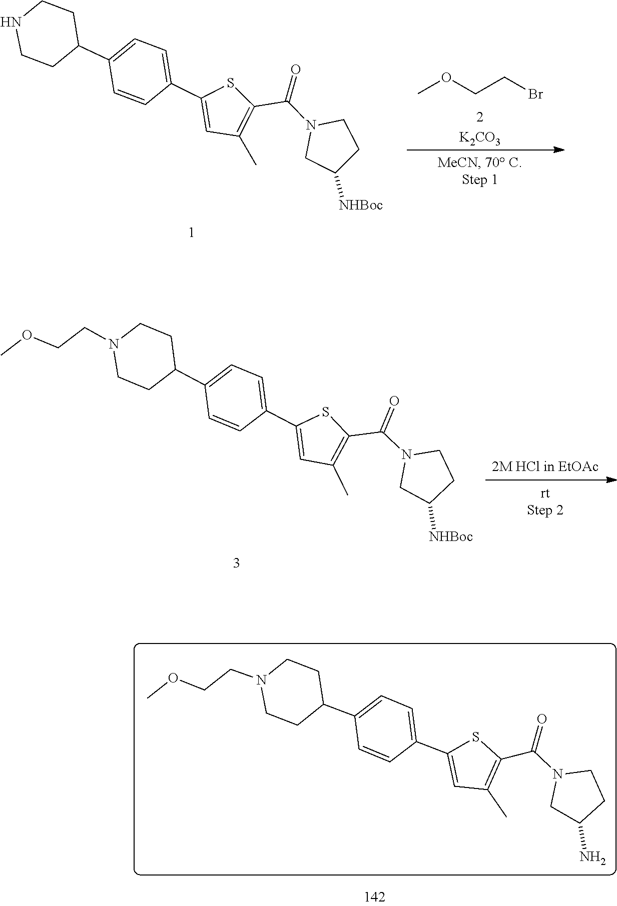

### Step 1 tert-butyl(S)-(1-(5-(4-(1-(2-methoxyethyl)piperidin-4-yl)phenyl)-3-methylthiophene-2-carbonyl)pyrrolidin-3-yl)carbamate

To the solution of tert-butyl (S)-(1-(3-methyl-5-(4-(piperidin-4-yl)phenyl)thiophene-2-carbonyl)pyrrolidin-3-yl)carbamate (300 mg, 0.6388 mmol) in MeCN (6 mL) were added 1-bromo-2-methoxyethane (177.57 mg, 1.2776 mmol) and K₂CO₃ (264.87 mg, 1.9164 mmol). The mixture was stirred at 70° C. for 16 h. The LCMS showed the reaction was completed and the desired product was found. The solvent was removed under reduced pressure and the residue was purified by flash chromatography (DCM/MeOH=95/5) to give tert-butyl (S)-(1-(5-(4-(1-(2-methoxyethyl)piperidin-4-yl)phenyl)-3-methylthiophene-2-carbonyl)pyrrolidin-3-yl)carbamate (110 mg, 29.37% yield) as a yellow solid.

### Step 2 (S)-(3-aminopyrrolidin-1-yl)(5-(4-(1-(2-methoxyethyl)piperidin-4-yl)phenyl)-3-methylthiophen-2-yl)methanone

To the solution of tert-butyl(S)-(1-(5-(4-(1-(2-methoxyethyl)piperidin-4-yl)phenyl)-3-methylthiophene-2-carbonyl)pyrrolidin-3-yl)carbamate (110 mg, 0.2084 mmol) in EtOAc (4 mL) was added HCl (3 ml, 2M in EtOAc). The mixture was stirred at rt for 16 h. The LCMS showed the reaction was completed and the desired product was found. The solvent was removed under reduced pressure and the residue was purified by prep-HPLC to give the desired product (Compound 142; 20 mg, 20.20% yield) as a yellow solid. MS (ESI): mass calc'd for C24H34N3O2S 428, m/z found 428 [M+H]⁺. 1H NMR (400 MHz, MeOD) δ 7.46 (d, J=8.0 Hz, 2H), 7.18 (d, J=8.0 Hz, 2H), 7.09 (s, 1H), 3.67 (m, 2H), 3.57-3.44 (m, 4H), 3.25 (t, J=3.6 Hz, 4H), 3.03 (d, J=11.2 Hz, 2H), 2.55 (t, J=5.6 Hz, 2H), 2.47 (m, 1H), 2.22 (s, 3H), 2.13 (m, 3H), 1.78-1.67 (m, 5H).

### Example 22: Synthesis of (S)-(3-aminopyrrolidin-1-yl)(3-methyl-5-(3-methyl-4-(1-methylpiperidin-4-yl)phenyl)thiophen-2-yl)methanone (Compound 144)

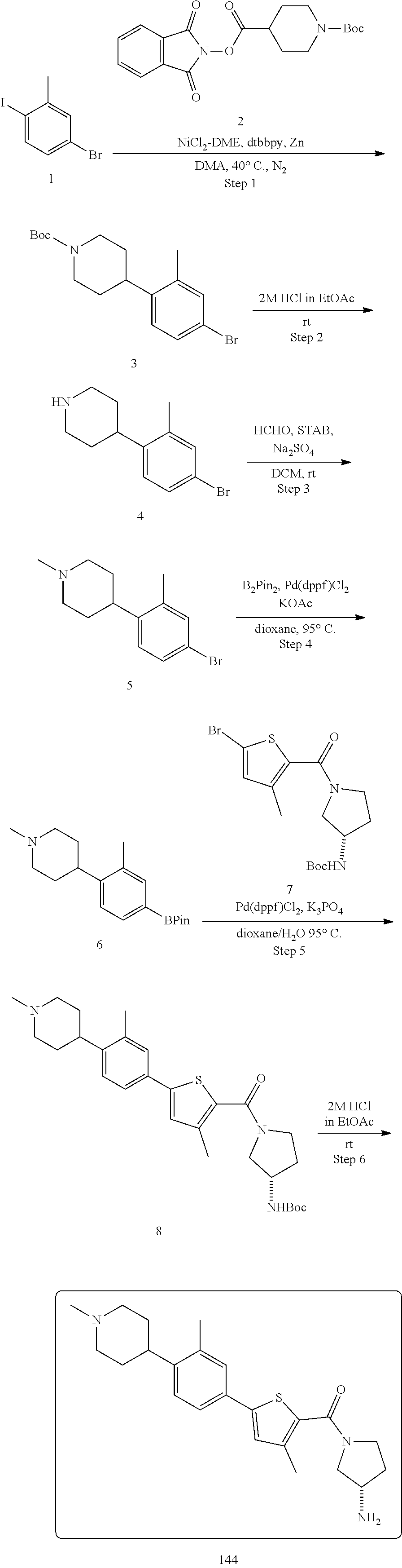

### Step 1 tert-butyl 4-(4-bromo-2-methylphenyl)piperidine-1-carboxylate

To a solution of NiCl₂-DME (0.15 g, 0.00068 mol) and dtbbpy (0.18 g, 0.00068 mol) in 15 mL DMA were added 4-bromo-1-iodo-2-methylbenzene (1 g, 0.0034 mol), 1-(tert-butyl) 4-(1,3-dioxoisoindolin-2-yl) piperidine-1,4-dicarboxylate (1.91 g, 0.0051 mol) and Zn powder (0.44 g, 0.0068 mol), the mixture was stirred at 40° C. under N₂ for 16 h. After the reaction, the H₂O was added and the mixture was extracted with EtOAc for 3 times. Combined with EA phases, washed with brine, dried over Na₂SO₄ and filtered, the solvent was removed under reduced pressure, the residue was purified by column chromatography to give tert-butyl 4-(4-bromo-2-methylphenyl)piperidine-1-carboxylate (0.4 g, 29.41% yield) as a yellow oil.

### Step 2 4-(4-bromo-2-methylphenyl)piperidine

To the solution of tert-butyl 4-(4-bromo-2-methylphenyl)piperidine-1-carboxylate (400 mg, 1.1258 mmol) in EtOAc (2 mL) was added HCl (5 mL, 2M in EtOAc). The mixture was stirred at rt for 16 h. The LCMS showed the reaction was completed and the desired MS was found. The mixture was filtered and concentrated under reduced pressure to give the desired product (280 mg, 88.07% yield) as a yellow solid.

### Step 3 4-(4-bromo-2-methylphenyl)-1-methylpiperidine

To the solution of 4-(4-bromo-2-methylphenyl)piperidine (280 mg, 1.1016 mmol) in DCM (15 mL) was added 37% HCHO (268.2 mg), Na₂SO₄ (93.9 mg, 0.6609 mmol) and NaBH(OAc)₃ (1400.8 mg, 6.6095 mmol). The mixture was stirred at rt for 16 h. The LCMS showed the reaction was completed and the desired MS was found. 15 mL NaHCO₃ was added and the mixture was extracted with EtOAc (3*20 mL). Combined the EtOAc phases, washed with brine, dried over anhydrous Na₂SO₄ and evaporated in vacuum. The residue was purified by silica gel column chromatography to give the desired product (210 mg, 63.97% yield) as a yellow oil.

### Step 4 1-methyl-4-(2-methyl-4-(4,4,5,5-tetramethyl-1,3,2-dioxaborolan-2-yl)phenyl)piperidine

To the solution of 4-(4-bromo-2-methylphenyl)-1-methylpiperidine (210 mg, 0.7830 mmol) in 1,4-dioxane (8 mL) were added B₂Pin₂ (218.7 mg, 0.8613 mmol), Pd(dppf)Cl₂DCM (127.8 mg, 0.1566 mmol) and KOAc (230.5 mg, 2.349 mmol), the mixture was stirred at 95° C. under N₂ for 16 h. The LCMS showed the reaction was completed and the desired MS was found. After cooled to rt, the mixture was filtered and concentrated under reduced pressure. The residue was purified by silica gel column chromatography to give the desired product (170 mg, 61.98% yield) as a yellow solid.

### Step 5 tert-butyl(S)-(1-(3-methyl-5-(3-methyl-4-(1-methylpiperidin-4-yl)phenyl)thiophene-2-carbonyl)pyrrolidin-3-yl)carbamate

To the solution of 1-methyl-4-(2-methyl-4-(4,4,5,5-tetramethyl-1,3,2-dioxaborolan-2-yl)phenyl)piperidine (170 mg, 0.5392 mmol) in dioxane/H₂O=8:1 (9 mL) was added tert-butyl (S)-(1-(5-bromo-3-methylthiophene-2-carbonyl)pyrrolidin-3-yl)carbamate (209.9 mg, 0.5392 mmol), K₃PO₄ (343.4 mg, 1.6176 mmol) and Pd(dppf)Cl₂DCM (88.0 mg, 0.1078 mmol). The mixture was stirred at 95° C. under N₂ for 16 h. The LCMS showed the reaction was completed and the desired MS was found. After cooled to rt, the mixture was filtered and concentrated under reduced pressure. The residue was purified by silica gel column chromatography to give the desired product (190 mg, 63.72% yield) as a yellow solid.

### Step 6 (S)-(3-aminopyrrolidin-1-yl)(3-methyl-5-(3-methyl-4-(1-methylpiperidin-4-yl)phenyl)thiophen-2-yl)methanone

To the solution of tert-butyl(S)-(1-(3-methyl-5-(3-methyl-4-(1-methylpiperidin-4-yl)phenyl)thiophene-2-carbonyl)pyrrolidin-3-yl)carbamate (190 mg, 0.3818 mmol) in EtOAc (3 mL) was added HCl (5 mL, 2M in EtOAc). The mixture was stirred at rt for 16 h. The LCMS showed the reaction was completed and the desired MS was found. The solvent was removed under reduced pressure and the residue was purified by prep-HPLC to give the desired product (Compound 144; 20 mg, 11.86% yield) as a yellow solid. MS (ESI): mass calc'd for C23H32N3OS 398, m/z found 398 [M+H]⁺. ¹H NMR (400 MHz, MeOD) δ 7.45 (d, J=8.0 Hz, 2H), 7.28 (d, J=7.6 Hz, 1H), 7.20 (s, 1H), 3.83-3.75 (m, 2H), 3.65 (m, 2H), 3.37 (d, J=5.2 Hz, 1H), 3.06 (d, J=11.6 Hz, 2H), 2.88-2.77 (m, 1H), 2.39 (d, J=6.0 Hz, 6H), 2.34 (s, 3H), 2.29-2.18 (m, 3H), 1.83 (m, 5H).

### Example 23: Synthesis of (S)-(3-aminopyrrolidin-1-yl)(3-methyl-5-(4-(1-((tetrahydro-2H-pyran-4-yl)methyl)piperidin-4-yl)phenyl)thiophen-2-yl)methanone (Compound 147)

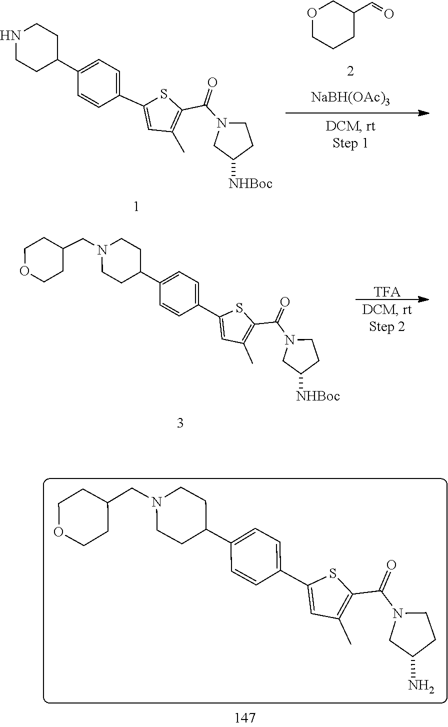 [SMILES: Cc1cc(-c2ccc(C3CCNCC3)cc2)sc1C(=O)N1CC[C@H](NC(=O)OC(C)(C)C)C1 ; O=CC1CCCOC1 ; Cc1cc(-c2ccc(C3CCN(CC4CCOCC4)CC3)cc2)sc1C(=O)N1CC[C@H](NC(=O)OC(C)(C)C)C1 ; Cc1cc(-c2ccc(C3CCN(CC4CCOCC4)CC3)cc2)sc1C(=O)N1CC[C@H](N)C1 ; "NaBH(OAc)3"]

### Step 1 tert-butyl(S)-(1-(3-methyl-5-(4-(1-((tetrahydro-2H-pyran-4-yl)methyl)piperidin-4-yl)phenyl)thiophene-2-carbonyl)pyrrolidin-3-yl)carbamate

To the solution of tert-butyl (S)-(1-(3-methyl-5-(4-(piperidin-4-yl)phenyl)thiophene-2-carbonyl)pyrrolidin-3-yl)carbamate (260 mg, 0.5536 mmol) in DCM (6 mL) was added oxane-4-carbaldehyde (442.32 mg, 3.8752 mmol), Sodium triacetoxyborohydride (175.99 mg, 0.8304 mmol) and HOAc (66.49 mg, 1.1072 mmol). The mixture was stirred at rt for 16 h. The LCMS showed the reaction was completed and the desired product was found. The solvent was removed under reduced pressure and the residue was purified by silica gel column chromatography to give the desired product (180 mg, 51.54% yield) as a yellow solid.

### Step 2 (S)-(3-aminopyrrolidin-1-yl)(3-methyl-5-(4-(1-((tetrahydro-2H-pyran-4-yl)methyl)piperidin-4-yl)phenyl)thiophen-2-yl)methanone

To the solution of tert-butyl(S)-(1-(3-methyl-5-(4-(1-((tetrahydro-2H-pyran-4-yl)methyl)piperidin-4-yl)phenyl)thiophene-2-carbonyl)pyrrolidin-3-yl)carbamate (180 mg, 0.3170 mmol) in DCM (5 mL) was added TFA (542.17 mg, 4.755 mmol). The mixture was stirred at rt for 16 h. The LCMS showed the reaction was completed and the desired MS was found. The solvent was removed under reduced pressure and the residue was purified by prep-HPLC to give the desired product (Compound 147; 30 mg, 18.20% yield) as a yellow solid. MS (ESI): mass calc'd for C27H37N3O2S 467, m/z found 468 [M+H]⁺. ¹H NMR (400 MHz, MeOD) δ 7.64 (d, J=8.0 Hz, 2H), 7.35 (d, J=8.0 Hz, 2H), 7.27 (s, 1H), 4.06-3.92 (m, 4H), 3.89-3.75 (m, 2H), 3.73-3.58 (m, 3H), 3.49 (t, J=11.6 Hz, 2H), 3.02 (d, J=5.6 Hz, 4H), 2.93 (t, J=11.6 Hz, 1H), 2.49-2.34 (m, 4H), 2.22-1.99 (m, 6H), 1.76 (d, J=12.8 Hz, 2H), 1.47-1.36 (m, 2H).

### Example 24: Synthesis of (S)-(3-aminopyrrolidin-1-yl)(5-(4-(4-cyclohexylpiperazin-1-yl)phenyl)-3-methylthiophen-2-yl)methanone (Compound 148)

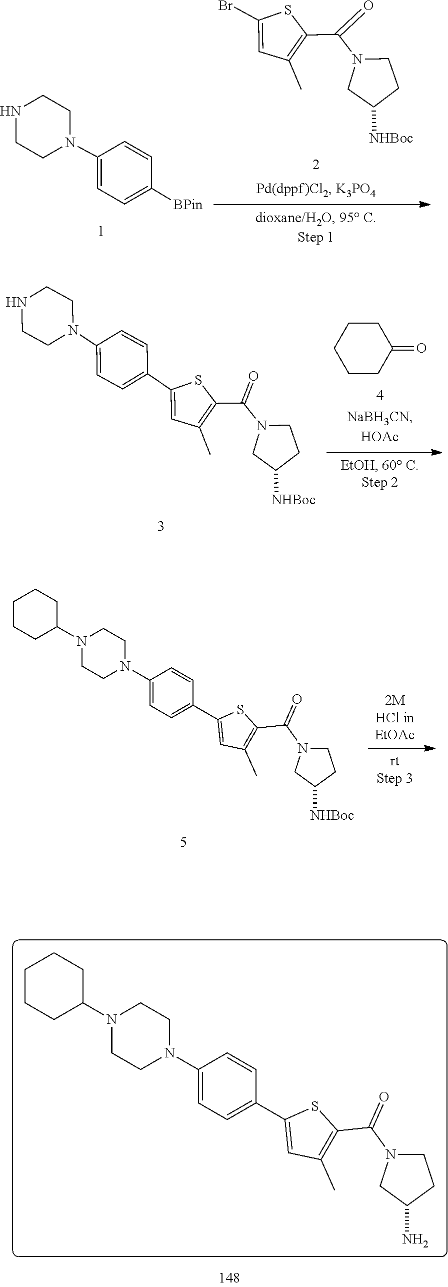

### Step 1 tert-butyl(S)-(1-(3-methyl-5-(4-(piperazin-1-yl)phenyl)thiophene-2-carbonyl)pyrrolidin-3-yl)carbamate

To the solution of 1-(4-(4,4,5,5-tetramethyl-1,3,2-dioxaborolan-2-yl)phenyl)piperazine (200 mg, 0.6940 mmol) in dioxane/H₂O=8:1 (9 mL) was added tert-butyl N-((3S)-1-((5-bromo-3-methylthiophen-2-yl)carbonyl)pyrrolidin-3-yl)carbamate (270.18 mg, 0.6940 mmol), K₃PO₄ (441.95 mg, 2.082 mmol) and Pd(dppf)Cl₂DCM (113.26 mg, 0.1388 mmol). The mixture was stirred at 95° C. under N₂ for 16 h. The LCMS showed the reaction was completed and the desired MS was found. After cooled to rt, the mixture was filtered and concentrated under reduced pressure. The residue was purified by silica gel column chromatography to give the desired product (130 mg, 35.82% yield) as a yellow solid.

### Step 2 tert-butyl(S)-(1-(5-(4-(4-cyclohexylpiperazin-1-yl)phenyl)-3-methylthiophene-2-carbonyl)pyrrolidin-3-yl)carbamate

To the solution of tert-butyl(S)-(1-(3-methyl-5-(4-(piperazin-1-yl)phenyl)thiophene-2-carbonyl)pyrrolidin-3-yl)carbamate (130 mg, 0.2762 mmol) in EtOH (5 mL) was added cyclohexanone (189.75 mg, 1.9334 mmol), NaBH₃CN (26.03 mg, 0.4143 mmol) and HOAc (33.17 mg, 0.5524 mmol). The mixture was stirred at 65° C. for 16 h. The LCMS showed the reaction was completed and the desired MS was found. The solvent was removed under reduced pressure and the residue was purified by silica gel column chromatography to give the desired product (90 mg, 53.04% yield) as a yellow solid.

### Step 3 (S)-(3-aminopyrrolidin-1-yl)(5-(4-(4-cyclohexylpiperazin-1-yl)phenyl)-3-methylthiophen-2-yl)methanone

To the solution of tert-butyl(S)-(1-(5-(4-(4-cyclohexylpiperazin-1-yl)phenyl)-3-methylthiophene-2-carbonyl)pyrrolidin-3-yl)carbamate (90 mg, 0.1628 mmol) in EtOAc (3 mL) was added HCl (4 mL, 2M in EtOAc). The mixture was stirred at rt for 16 h. The LCMS showed the reaction was completed and the desired MS was found. The mixture was filtered and concentrated under reduced pressure to give the desired product (Compound 148; 40 mg, 48.83% yield) as a yellow solid. MS (ESI): mass calc'd for C26H37N4OS 453, m/z found 453 [M+H]⁺. ¹H NMR (400 MHz, MeOD) δ 7.48 (d, J=8.8 Hz, 2H), 7.06 (s, 1H), 6.99 (d, J=8.8 Hz, 2H), 3.88 (dd, J=15.6, 10.8 Hz, 4H), 3.82-3.64 (m, 2H), 3.59 (dd, J=15.6, 10.4 Hz, 3H), 3.26 (s, 1H), 3.16 (s, 1H), 3.09 (t, J=12.4 Hz, 2H), 2.35 (td, J=13.6, 7.2 Hz, 1H), 2.25 (s, 3H), 2.12 (d, J=11.2 Hz, 2H), 2.04 (dd, J=11.6, 6.4 Hz, 1H), 1.89 (t, J=12.8 Hz, 2H), 1.64 (d, J=12.8 Hz, 1H), 1.57-1.06 (m, 6H).

### Example 25: Synthesis of (S)-(3-aminopyrrolidin-1-yl)(3-methyl-5-(3-methyl-4-(piperidin-4-yl)phenyl)thiophen-2-yl)methanone (Compound 149)

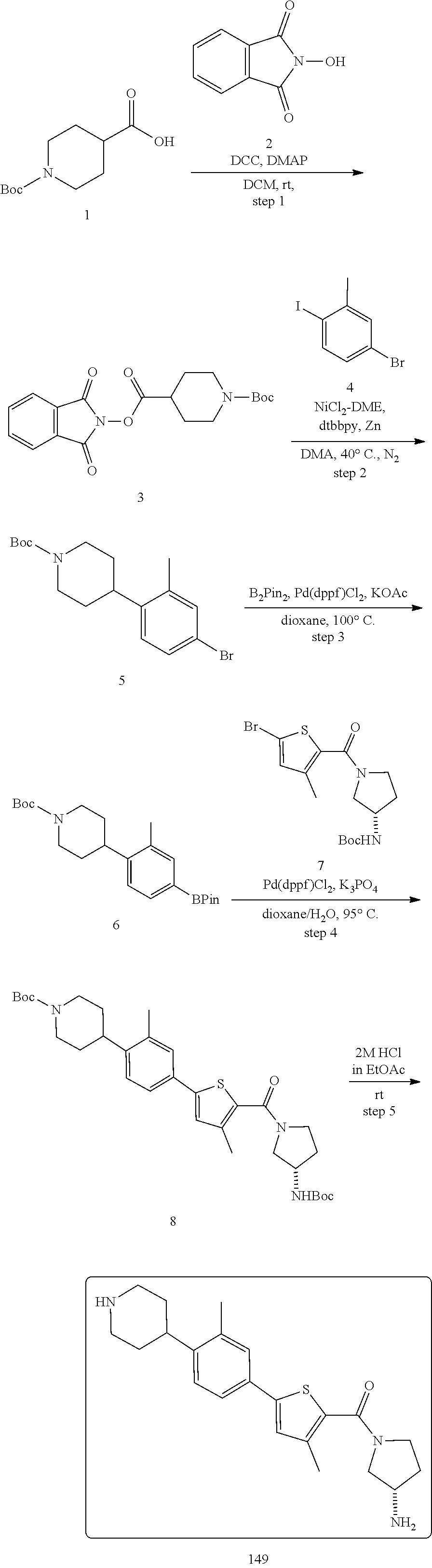

### Step 1 Synthesis of 1-(tert-butyl) 4-(1,3-dioxoisoindolin-2-yl) piperidine-1,4-dicarboxylate

To a solution of 1-(tert-butoxycarbonyl)piperidine-4-carboxylic acid (10 g, 43.4 mmol), 2-hydroxyisoindole-1,3-dione (7.08 g, 43.4 mmol) and DMAP (0.27 g, 2.1 mmol) in 60 mL DCM was added DCC in 40 mL DCM, the mixture was stirred at 25° C. for 16 hours. After the reaction, the solvent was removed under reduced pressure, the residue was purified by column chromatography to afford 1-(tert-butyl) 4-(1,3-dioxoisoindolin-2-yl) piperidine-1,4-dicarboxylate (10.7 g, yield=64.98%) as a white solid. ¹H NMR (400 MHz, CDCl3) δ 7.90-7.87 (m, 2H), 7.82-7.79 (m, 2H), 4.04 (d, J=12.0 Hz, 2H), 3.05-2.98 (m, 2H), 2.95-2.88 (m, 1H), 2.19-2.05 (m, 2H), 1.89-1.81 (m, 2H), 1.47 (s, 9H).

### Step 2 Synthesis of tert-butyl 4-(4-bromo-2-methylphenyl)piperidine-1-carboxylate

To a solution of NiCl₂·DME (0.15 g, 0.6 mmol) and dtbbpy (0.2 g, 0.7 mmol) in 10 mL DMA were added 4-bromo-1-iodo-2-methylbenzene (1 g, 3.4 mmol), 1-(tert-butyl) 4-(1,3-dioxoisoindolin-2-yl) piperidine-1,4-dicarboxylate (1.91 g, 5.1 mmol) and Zn powder (0.44 g, 6.8 mmol), the mixture was stirred at 40° C. under N₂ for 16 hours. After the reaction, the H₂O was added and the mixture was extracted with EtOAc for 3 times. Combined with EtOAc phases, washed with brine, dried over Na₂SO₄ and filtered, the solvent was removed under reduced pressure, the residue was purified by column chromatography to afford tert-butyl 4-(4-bromo-2-methylphenyl)piperidine-1-carboxylate (0.73 g, yield=58.82%) as a colorless oil. LCMS (ESI) calc'd for C16H22BrNO2+ [M-Me+H] m/z 339.08, found 339.0.

### Step 3 Synthesis of tert-butyl 4-(2-methyl-4-(4,4,5,5-tetramethyl-1,3,2-dioxaborolan-2-yl)phenyl)piperidine-1-carboxylate

To a solution of tert-butyl 4-(4-bromo-2-methylphenyl)piperidine-1-carboxylate (730 mg, 2.0547 mmol), B₂Pin₂ (626.2 mg, 2.4656 mmol) and KOAc (605 mg, 6.1641 mmol) in 10 mL dioxane was added Pd(dppf)Cl₂DCM (335.6 mg, 0.4109 mmol), the mixture was stirred at 100° C. under N₂ for 16 hours. After the reaction, the solvent was removed under reduced pressure, the residue was purified by column chromatography to afford tert-butyl 4-(2-methyl-4-(4,4,5,5-tetramethyl-1,3,2-dioxaborolan-2-yl)phenyl)piperidine-1-carboxylate (446 mg, 53% yield) as a white solid. LCMS (ESI): calc'd. for C19H29BNO4+ [M-^{t}Bu+2H] m/z 346.22, found 346.1.

### Step 4 Synthesis of tert-butyl (S)-4-(4-(5-(3-((tert-butoxycarbonyl)amino)pyrrolidine-1-carbonyl)-4-methylthiophen-2-yl)-2-methylphenyl)piperidine-1-carboxylate

To a solution of tert-butyl (S)-(1-(5-bromo-3-methylthiophene-2-carbonyl)pyrrolidin-3-yl)carbamate (120 mg, 0.3082 mmol), tert-butyl4-(2-methyl-4-(4,4,5,5-tetramethyl-1,3,2-dioxaborolan-2-yl)phenyl)piperidine-1-carboxylate (148.8 mg, 0.3698 mmol) and K₃PO₄ (196.3 mg, 0.9245 mmol) in dioxane/H₂O (9 mL, v:v=8:1) was added Pd(dppf)Cl₂DCM (50.3 mg, 0.0616 mmol), the mixture was stirred at 95° C. under N₂ for 16 hours. After the reaction, the solvent was removed under reduced pressure, the residue was purified by column chromatography to afford tert-butyl (S)-4-(4-(5-(3-((tert-butoxycarbonyl)amino)pyrrolidine-1-carbonyl)-4-methylthiophen-2-yl)-2-methylphenyl)piperidine-1-carboxylate (82 mg, 45.04% yield) as a yellow solid. LCMS (ESI) calc'd for C32H46N3O5S+ [M+H] m/z 584.32, found 584.3.

### Step 5 Synthesis of (S)-(3-aminopyrrolidin-1-yl)(3-methyl-5-(3-methyl-4-(piperidin-4-yl)phenyl)thiophen-2-yl)methanone

To a solution of tert-butyl (S)-4-(4-(5-(3-((tert-butoxycarbonyl)amino)pyrrolidine-1-carbonyl)-4-methylthiophen-2-yl)-2-methylphenyl)piperidine-1-carboxylate (82 mg, 0.1402 mmol) in 2 mL EtOAc was added HCl (4 mL, 2M in EtOAc), the mixture was stirred at 25° C. for 12 hours. After the reaction, the mixture was filtered and the filter cake was washed with EtOAc for several times to afford (S)-(3-aminopyrrolidin-1-yl)(3-methyl-5-(3-methyl-4-(piperidin-4-yl)phenyl)thiophen-2-yl)methanone (Compound 149; 20.2 mg, 34.95% yield) as a yellow solid. LCMS (ESI) calc'd for C22H30N3OS+ [M+H] m/z 384.21, found 384.1. ¹H NMR (400 MHz, MeOD) δ 7.49-7.46 (m, 2H), 7.28 (d, J=7.5 Hz, 1H), 7.23 (s, 2H), 4.02 (s, 2H), 3.85-3.71 (m, 3H), 3.52 (d, J=10.5 Hz, 2H), 3.20 (s, 3H), 2.50-2.42 (m, 4H), 2.35 (s, 3H), 2.20-2.12 (m, 1H), 2.01-1.94 (m, 4H).

### Example 26: Synthesis of (S)-(3-aminopyrrolidin-1-yl)(3-methyl-5-(4-(4-(tetrahydro-2H-pyran-4-yl)piperazin-1-yl)phenyl)thiophen-2-yl)methanone (Compound 151)

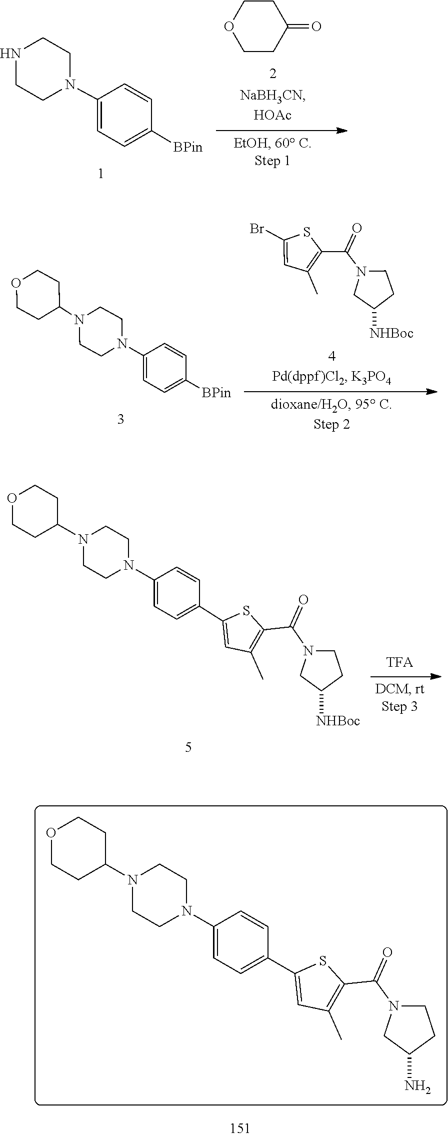

### Step 1 1-(tetrahydro-2H-pyran-4-yl)-4-(4-(4,4,5,5-tetramethyl-1,3,2-dioxaborolan-2-yl)phenyl)piperazine

To the solution of 1-(4-(4,4,5,5-tetramethyl-1,3,2-dioxaborolan-2-yl)phenyl)piperazine (700 mg, 2.4289 mmol) in EtOH (10 mL) was added oxan-4-one (1702.2 mg, 17.002 mmol), NaBH₃CN (228.9 mg, 3.6433 mmol) and HOAc (291.7 mg, 4.8578 mmol). The mixture was stirred at 65° C. for 16 h. The LCMS showed the reaction was completed and the desired MS was found. The solvent was removed under reduced pressure and the residue was purified by flash chromatography (DCM/MeOH=95/5) to give 1-(tetrahydro-2H-pyran-4-yl)-4-(4-(4,4,5,5-tetramethyl-1,3,2-dioxaborolan-2-yl)phenyl)piperazine (500 mg, 49.76% yield) as a yellow solid.

### Step 2 tert-butyl(S)-(1-(3-methyl-5-(4-(4-(tetrahydro-2H-pyran-4-yl)piperazin-1-yl)phenyl)thiophene-2-carbonyl)pyrrolidin-3-yl)carbamate

To the solution of 1-(tetrahydro-2H-pyran-4-yl)-4-(4-(4,4,5,5-tetramethyl-1,3,2-dioxaborolan-2-yl)phenyl)piperazine (300 mg, 0.8058 mmol) in dioxane/H₂O=8:1 (9 mL) was added tert-butyl N-((3S)-1-((5-bromo-3-methylthiophen-2-yl)carbonyl)pyrrolidin-3-yl)carbamate (313.7 mg, 0.8058 mmol), K₃PO₄ (513.1 mg, 2.4173 mmol) and Pd(dppf)Cl₂DCM (131.5 mg, 0.1611 mmol). The mixture was stirred at 95° C. under N₂ for 16 h. The LCMS showed the reaction was completed and the desired MS was found. After cooled to rt, the mixture was filtered and concentrated under reduced pressure. The residue was purified by silica gel column chromatography to give the desired product (180 mg, 36.24% yield) as a yellow solid.

### Step 3 (S)-(3-aminopyrrolidin-1-yl)(3-methyl-5-(4-(4-(tetrahydro-2H-pyran-4-yl)piperazin-1-yl)phenyl)thiophen-2-yl)methanone

To the solution of tert-butyl(S)-(1-(3-methyl-5-(4-(4-(tetrahydro-2H-pyran-4-yl)piperazin-1-yl)phenyl)thiophene-2-carbonyl)pyrrolidin-3-yl)carbamate (180 mg, 0.3245 mmol) in DCM (5 mL) was added TFA (555 mg, 4.8675 mmol). The mixture was stirred at rt for 16 h. The LCMS showed the reaction was completed and the desired MS was found. The solvent was removed under reduced pressure and the residue was purified by prep-HPLC to give the desired product (Compound 151; 50 mg, 32.20% yield) as a yellow solid. MS (ESI): mass calc'd for C25H35N4O2S 455, m/z found 455 [M+H]⁺. ¹H NMR (400 MHz, MeOD) δ 7.53 (d, J=8.8 Hz, 2H), 7.10 (s, 1H), 7.00 (d, J=8.8 Hz, 2H), 4.03 (dd, J=11.2, 3.6 Hz, 2H), 3.79 (dt, J=13.6, 6.8 Hz, 2H), 3.72-3.55 (m, 2H), 3.45 (t, J=11.6 Hz, 2H), 3.37 (d, J=5.6 Hz, 1H), 3.31-3.24 (m, 4H), 2.88-2.71 (m, 4H), 2.51 (t, J=11.6 Hz, 1H), 2.33 (s, 3H), 2.18 (s, 1H), 1.92 (d, J=11.6 Hz, 2H), 1.83 (td, J=13.2, 6.8 Hz, 1H), 1.68-1.47 (m, 2H).

### Example 27: Synthesis of ((S)-3-aminopyrrolidin-1-yl)(3-methyl-5-(4-(4-(tetrahydrofuran-3-yl)piperazin-1-yl)phenyl)thiophen-2-yl)methanone (Compound 152)

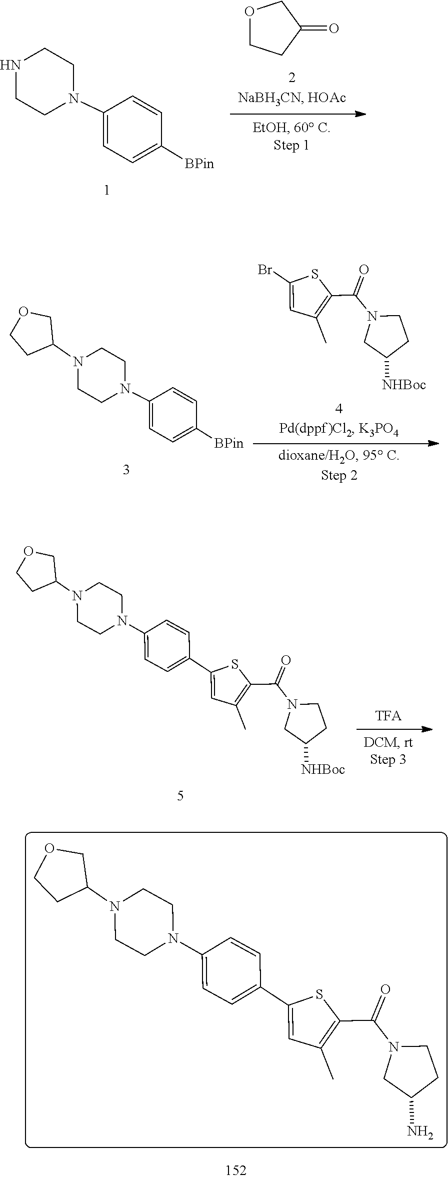

### Step 1 1-(tetrahydrofuran-3-yl)-4-(4-(4,4,5,5-tetramethyl-1,3,2-dioxaborolan-2-yl)phenyl)piperazine

To the solution of 1-(4-(4,4,5,5-tetramethyl-1,3,2-dioxaborolan-2-yl)phenyl)piperazine (700 mg, 2.4289 mmol) in EtOH (10 mL) was added oxolan-3-one (1463.7 mg, 17.002 mmol), NaBH₃CN (228.9 mg, 3.6433 mmol) and HOAc (291.7 mg, 4.8578 mmol). The mixture was stirred at 65° C. for 16 h. The LCMS showed the reaction was completed and the desired MS was found. The solvent was removed under reduced pressure and the residue was purified by flash chromatography (DCM/MeOH=95/5) to give 1-(tetrahydrofuran-3-yl)-4-(4-(4,4,5,5-tetramethyl-1,3,2-dioxaborolan-2-yl)phenyl)piperazine (500 mg, 51.71% yield) as a yellow solid.

### Step 2 tert-butyl((3S)-1-(3-methyl-5-(4-(4-(tetrahydrofuran-3-yl)piperazin-1-yl)phenyl)thiophene-2-carbonyl)pyrrolidin-3-yl)carbamate

To the solution of 1-(tetrahydrofuran-3-yl)-4-(4-(4,4,5,5-tetramethyl-1,3,2-dioxaborolan-2-yl)phenyl)piperazine (250 mg, 0.6978 mmol) in dioxane/H₂O=8:1 (9 mL) was added tert-butyl N-((3S)-1-((5-bromo-3-methylthiophen-2-yl)carbonyl)pyrrolidin-3-yl)carbamate (271.7 mg, 0.6978 mmol), K₃PO₄ (444.4 mg, 2.0934 mmol) and Pd(dppf)Cl₂DCM (113.9 mg, 0.1395 mmol). The mixture was stirred at 95° C. under N₂ for 16 h. The LCMS showed the reaction was completed and the desired MS was found. After cooled to rt, the mixture was filtered and concentrated under reduced pressure. The residue was purified by silica gel column chromatography to give the desired product (180 mg, 42.93% yield) as a yellow solid.

### Step 3 ((S)-3-aminopyrrolidin-1-yl)(3-methyl-5-(4-(4-(tetrahydrofuran-3-yl)piperazin-1-yl)phenyl)thiophen-2-yl)methanone

To the solution of tert-butyl((3S)-1-(3-methyl-5-(4-(4-(tetrahydrofuran-3-yl)piperazin-1-yl)phenyl)thiophene-2-carbonyl)pyrrolidin-3-yl)carbamate (180 mg, 0.3329 mmol) in DCM (5 mL) was added TFA (569.4 mg, 4.9935 mmol). The mixture was stirred at rt for 16 h. The LCMS showed the reaction was completed and the desired MS was found. The solvent was removed under reduced pressure and the residue was purified by prep-HPLC to give the desired product (Compound 152; 40 mg, 24.54% yield) as a yellow solid. MS (ESI): mass calc'd for C24H33N4O2S 441, m/z found 441 [M+H]+. ¹H NMR (400 MHz, MeOD) δ 7.53 (d, J=8.8 Hz, 2H), 7.10 (s, 1H), 7.00 (d, J=8.8 Hz, 2H), 4.03-3.89 (m, 2H), 3.86-3.75 (m, 3H), 3.73-3.55 (m, 3H), 3.37 (d, J=5.2 Hz, 1H), 3.28 (t, J=5.2 Hz, 4H), 3.13-3.00 (m, 1H), 2.75 (dt, J=10.4, 4.8 Hz, 2H), 2.71-2.59 (m, 2H), 2.33 (s, 3H), 2.16 (dd, J=12.0, 4.4 Hz, 2H), 1.92 (dd, J=12.4, 7.6 Hz, 1H), 1.88-1.77 (m, 1H).

### Example 28: Synthesis of (S)-(3-aminopyrrolidin-1-yl)(5-(4-(4-cyclopentylpiperazin-1-yl)phenyl)-3-methylthiophen-2-yl)methanone (Compound 153)

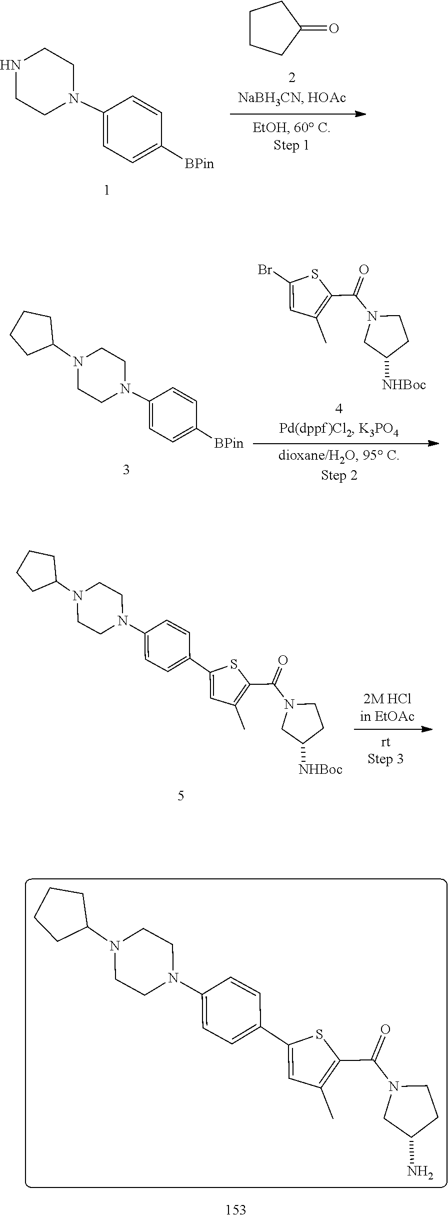

### Step 1 Synthesis of 1-cyclopentyl-4-(4-(4,4,5,5-tetramethyl-1,3,2-dioxaborolan-2-yl)phenyl)piperazine

To a solution of 1-(4-(4,4,5,5-tetramethyl-1,3,2-dioxaborolan-2-yl)phenyl)piperazine (288 mg, 1 mmol) in EtOH (10 mL) were added cyclopentanone (84 mg, 1 mmol), NaBH₃CN (94.2 mg, 1.5 mmol) and AcOH (60 mg, 1 mmol). The mixture was stirred at 65° C. for 16 h. The LCMS showed the reaction was completed and the desired MS was found. After cooled to rt, the resulted mixture was concentrated under reduced pressure. The residue was purified by flash chromatography (petroleum ether/EtOAc=3/7) to give 1-cyclopentyl-4-(4-(4,4,5,5-tetramethyl-1,3,2-dioxaborolan-2-yl)phenyl)piperazine (208 mg, 55.5% yield) as a white solid.

LCMS (ESI) calc'd for C21H34BN2O2+ [M+H]+ m/z 357.27, found 357.

### Step 2 Synthesis of tert-butyl (S)-(1-(5-(4-(4-cyclopentylpiperazin-1-yl)phenyl)-3-methylthiophene-2-carbonyl)pyrrolidin-3-yl)carbamate

To a solution of tert-butyl (S)-(1-(5-bromo-3-methylthiophene-2-carbonyl)pyrrolidin-3-yl)carbamate (227 mg, 0.58 mmol) in dioxane/H₂O=8:1 (18 mL) were added 1-cyclopentyl-4-(4-(4,4,5,5-tetramethyl-1,3,2-dioxaborolan-2-yl)phenyl)piperazine (207.8 mg, 0.58 mmol), K₃PO₄ (371.3 mg, 1.75 mmol) and Pd(dppf)Cl₂DCM (95.2 mg, 0.12 mmol). The mixture was stirred at 95° C. under N₂ for 16 h. After the reaction, the mixture was filtered and concentrated under reduced pressure. The residue was purified by flash chromatography (ethyl acetate 100%) to give tert-butyl (S)-(1-(5-(4-(4-cyclopentylpiperazin-1-yl)phenyl)-3-methylthiophene-2-carbonyl)pyrrolidin-3-yl)carbamate (150 mg, 45.4% yield) as a white solid. LCMS (ESI) calc'd for C30H43N4O3S+ [M+H]+ m/z 539.31, found 539.

### Step 3 Synthesis of (S)-(3-aminopyrrolidin-1-yl)(5-(4-(4-cyclopentylpiperazin-1-yl)phenyl)-3-methylthiophen-2-yl)methanone

To a solution of tert-butyl (S)-(1-(5-(4-(4-cyclopentylpiperazin-1-yl)phenyl)-3-methylthiophene-2-carbonyl)pyrrolidin-3-yl)carbamate (150 mg, 0.28 mmol) in 4 mL EtOAc was added HCl (3 mL, 2M in EtOAc). The mixture was stirred at rt for 16 h. The LCMS showed the reaction was completed and the desired product was found. The mixture was concentrated under reduced pressure. The residue was purified by prep-HPLC to give (S)-(3-aminopyrrolidin-1-yl)(5-(4-(4-cyclopentylpiperazin-1-yl)phenyl)-3-methylthiophen-2-yl)methanone (Compound 153; 53.3 mg, 39.3% yield) as a yellow solid. LCMS (ESI) calc'd for C26H37N3OS+ [M+H]+ m/z 439.27, found 438. ¹H NMR (400 MHz, MeOD) δ 7.60 (d, J=5.1 Hz, 2H), 7.17 (s, 1H), 7.11 (d, J=5.4 Hz, 2H), 4.03 (s, 2H), 3.95 (d, J=9.0 Hz, 2H), 3.86 (s, 1H), 3.74 (s, 4H), 3.65 (s, 1H), 3.28 (s, 4H), 2.46 (s, 1H), 2.36 (s, 3H), 2.24 (s, 4H), 1.89 (s, 4H), 1.73 (s, 2H).

### Example 29: Synthesis of (S)-(3-aminopyrrolidin-1-yl)(5-(4-(4-ethylpiperazin-1-yl)phenyl)-3-methylthiophen-2-yl)methanone (Compound 155)

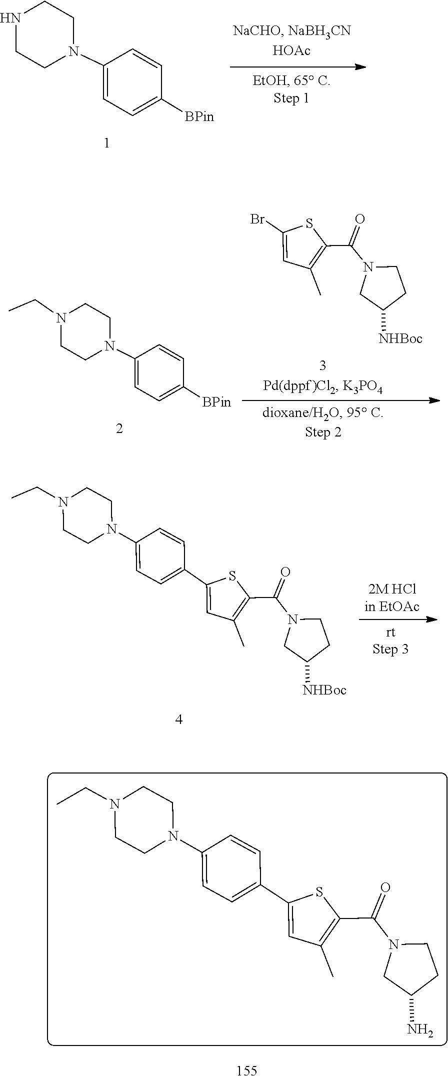

### Step 1 1-ethyl-4-(4-(4,4,5,5-tetramethyl-1,3,2-dioxaborolan-2-yl)phenyl)piperazine

To a mixture of 1-(4-(4,4,5,5-tetramethyl-1,3,2-dioxaborolan-2-yl)phenyl)piperazine (400 mg, 1.39 mmol) and acetaldehyde (306 mg, 6.94 mmol) in EtOH (15 mL) was added AcOH (167 mg, 2.78 mmol) and sodium cyanoborohydride (262 mg, 4.16 mmol), then it was stirred at 65° C. for 6 hours. LCMS showed the reaction was completed. The reaction mixture was concentrated to dryness. The crude product was purified by column chromatography on silica gel (MeOH/DCM=4%~5%) to give the desired product (380 mg, 77.9% yield) as light-yellow oil.

### Step 2 tert-butyl (S)-(1-(5-(4-(4-ethylpiperazin-1-yl)phenyl)-3-methylthiophene-2-carbonyl)pyrrolidin-3-yl)carbamate

To a mixture of compound 2 (270 mg, 0.85 mmol) and compound 3 (332 mg, 0.85 mmol) in 1,4-dioxane (8 mL) and H₂O (1 mL) was added K₃PO₄ (544 mg, 2.56 mmol) and Pd(dppf)Cl₂ (139 mg, 0.17 mmol), then it was stirred at 95° C. under N₂ for 16 hours. LCMS showed the reaction was completed. The reaction mixture was concentrated to dryness. The crude product was purified by column chromatography on silica gel (EtOAc/PE=0%~100%) to afford compound 4 (300 mg, 63.4% yield) as a brown solid.

### Step 3 (S)-(3-aminopyrrolidin-1-yl)(5-(4-(4-ethylpiperazin-1-yl)phenyl)-3-methylthiophen-2-yl)methanone

A mixture of compound 4 (300 mg, 0.60 mmol) in HCl/EtOAc (3 mL) was stirred at 25° C. for 3 hours. LCMS showed the reaction was complete. The reaction mixture was concentrated to dryness. The crude product was purified by prep-HPLC to afford desired product (Compound 155; 117.6 mg, 48.1% yield) as a white solid. LCMS (ESI): mass calc'd. for C22H31N4OS 399.22, m/z found 399.2 [M+H]⁺. ¹H NMR (400 MHz, MeOD) δ 7.50 (d, J=8.4 Hz, 2H), 7.07 (s, 1H), 6.97 (d, J=8.4 Hz, 2H), 3.82-3.70 (m, 2H), 3.68-3.51 (m, 2H), 3.36-3.31 (m, 1H), 3.28-3.24 (m, 4H), 2.70-2.60 (m, 4H), 2.54-2.45 (m, 2H), 2.31 (s, 3H), 2.15 (s, 1H), 1.87-1.72 (m, 1H), 1.15 (t, J=7.2 Hz, 3H).

### Example 30: Synthesis of (S)-(3-aminopyrrolidin-1-yl)(5-(4-(1-(1,1-dioxidotetrahydro-2H-thiopyran-4-yl)piperidin-4-yl)phenyl)-3-methylthiophen-2-yl)methanone (Compound 156)

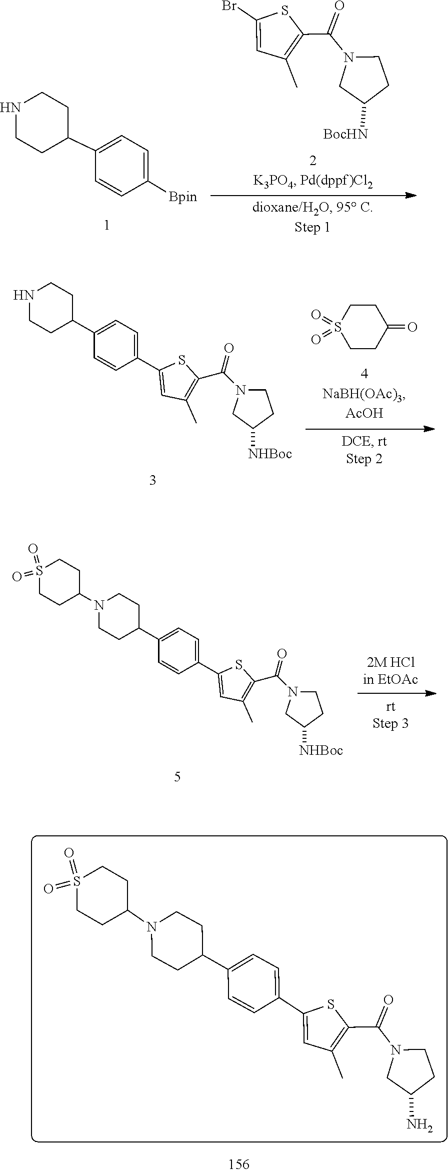

### Step 1 tert-butyl(S)-(1-(3-methyl-5-(4-(piperidin-4-yl)phenyl)thiophene-2-carbonyl)pyrrolidin-3-yl)carbamate

To the solution of 4-(4-(4,4,5,5-tetramethyl-1,3,2-dioxaborolan-2-yl)phenyl)piperidine (600 mg, 2.0891 mmol) in dioxane/H₂O=8:1 (18 mL) were added tert-butyl N-((3S)-1-((5-bromo-3-methylthiophen-2-yl)carbonyl)pyrrolidin-3-yl)carbamate (813.30 mg, 2.0891 mmol), K₃PO₄ (1330.36 mg, 6.2673 mmol) and Pd(dppf)Cl₂DCM (340.94 mg, 0.4178 mmol). The mixture was stirred at 95° C. under N₂ for 16 h. The LCMS showed the reaction was completed and the desired product was found. After cooled to rt, the mixture was filtered and concentrated under reduced pressure. The residue was purified by silica gel column chromatography (EA:MeOH=2:1) to give the desired product (700 mg, 67.78% yield) as a yellow solid.

### Step 2 tert-butyl(S)-(1-(5-(4-(1-(1,1-dioxidotetrahydro-2H-thiopyran-4-yl)piperidin-4-yl)phenyl)-3-methylthiophene-2-carbonyl)pyrrolidin-3-yl)carbamate

To the solution of tetrahydro-4H-thiopyran-4-one 1,1-dioxide (63.11 mg, 0.4259 mmol) in DCE (8 mL) were added tert-butyl(S)-(1-(3-methyl-5-(4-(piperidin-4-yl)phenyl)thiophene-2-carbonyl)pyrrolidin-3-yl)carbamate (200 mg, 0.4259 mmol), NaBH(OAc)₃ (126.37 mg, 0.5962 mmol) and HOAc (25.6 mg, 0.4259 mmol). The mixture was stirred at 40° C. for 16 h. The LCMS showed the reaction was completed and the desired product was found. The solvent was removed under reduced pressure and the residue was purified by prep-HPLC to give the desired product (50 mg, 17.55% yield) as a yellow solid.

### Step 3 (S)-(3-aminopyrrolidin-1-yl)(5-(4-(1-(1,1-dioxidotetrahydro-2H-thiopyran-4-yl)piperidin-4-yl)phenyl)-3-methylthiophen-2-yl)methanone

To the solution of tert-butyl(S)-(1-(5-(4-(1-(1,1-dioxidotetrahydro-2H-thiopyran-4-yl)piperidin-4-yl)phenyl)-3-methylthiophene-2-carbonyl)pyrrolidin-3-yl)carbamate (50 mg, 0.0831 mmol) in EtOAc (4 mL) was added HCl (2 mL, 2M in EtOAc). The mixture was stirred at rt for 16 h. The LCMS showed the reaction was completed and the desired product was found. The mixture was filtered and concentrated under reduced pressure to give the desired product (Compound 156; 23 mg, 49.70% yield) as a solid. MS (ESI): mass calc'd for C26H36N3O3S2 502, m/z found 502 [M+H]⁺. ¹H NMR (400 MHz, MeOD) δ 7.53 (d, J=8.0 Hz, 2H), 7.25 (d, J=8.0 Hz, 2H), 7.15 (s, 1H), 3.89 (t, J=7.6 Hz, 2H), 3.80-3.64 (m, 2H), 3.65-3.49 (m, 4H), 3.31-3.21 (m, 4H), 3.15 (d, J=12.8 Hz, 2H), 2.86 (d, J=7.6 Hz, 1H), 2.49 (d, J=12.4 Hz, 2H), 2.41-2.21 (m, 6H), 2.08 (d, J=6.4 Hz, 5H).

### Example 31: Synthesis of (S)-(3-aminopyrrolidin-1-yl)(5-(4-(4-cyclobutylpiperazin-1-yl)phenyl)-3-methylthiophen-2-yl)methanone (Compound 158)

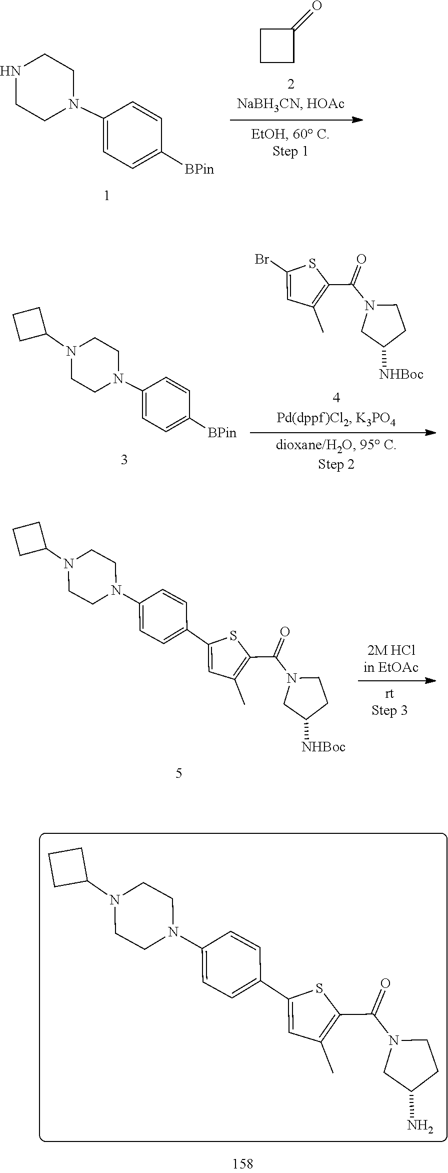

### Step 1 1-cyclobutyl-4-(4-(4,4,5,5-tetramethyl-1,3,2-dioxaborolan-2-yl)phenyl)piperazine

To the solution of 1-(4-(4,4,5,5-tetramethyl-1,3,2-dioxaborolan-2-yl)phenyl)piperazine (700 mg, 2.4289 mmol) in EtOH (10 mL) were added cyclobutanone (1191.69 mg, 17.002 mmol), NaBH₃CN (228.95 mg, 3.6433 mmol) and HOAc (291.71 mg, 4.8578 mmol). The mixture was stirred at 65° C. for 16 h. The LCMS showed the reaction was completed and the desired MS was found. The solvent was removed under reduced pressure and the residue was purified by flash chromatography (DCM/MeOH=95/5) to give 1-cyclobutyl-4-(4-(4,4,5,5-tetramethyl-1,3,2-dioxaborolan-2-yl)phenyl)piperazine (650 mg, 70.37% yield) as a yellow solid.

### Step 2 tert-butyl(S)-(1-(5-(4-(4-cyclobutylpiperazin-1-yl)phenyl)-3-methylthiophene-2-carbonyl)pyrrolidin-3-yl)carbamate

To the solution of 1-cyclobutyl-4-(4-(4,4,5,5-tetramethyl-1,3,2-dioxaborolan-2-yl)phenyl)piperazine (300 mg, 0.8765 mmol) in dioxane/H₂O=8:1 (9 mL) was added tert-butyl N-((3S)-1-((5-bromo-3-methylthiophen-2-yl)carbonyl)pyrrolidin-3-yl)carbamate (341.23 mg, 0.8765 mmol), K₃PO₄ (558.16 mg, 2.6295 mmol) and Pd(dppf)Cl₂DCM (143.04 mg, 0.1753 mmol). The mixture was stirred under N₂ at 95° C. for 16 h. The LCMS showed the reaction was completed and the desired MS was found. After cooled to rt, the mixture was filtered and concentrated under reduced pressure. The residue was purified by silica gel column chromatography to give the desired product (150 mg, 29.36% yield) as a yellow solid.

### Step 3 (S)-(3-aminopyrrolidin-1-yl)(5-(4-(4-cyclobutylpiperazin-1-yl)phenyl)-3-methylthiophen-2-yl)methanone

To the solution of tert-butyl(S)-(1-(5-(4-(4-cyclobutylpiperazin-1-yl)phenyl)-3-methylthiophene-2-carbonyl)pyrrolidin-3-yl)carbamate (100 mg, 0.1906 mmol) in EtOAc (2 mL) was added HCl (3 mL, 2M in EtOAc). The mixture was stirred at rt for 16 h. The LCMS showed the reaction was completed and the desired MS was found. The mixture was filtered and concentrated under reduced pressure to give the desired product (Compound 158; 20 mg, 22.25% yield) as a yellow solid. MS (ESI): mass calc'd for C24H33N4OS 425, m/z found 425 [M+H]. ¹H NMR (400 MHz, MeOD) δ 7.48 (d, J=8.4 Hz, 2H), 7.05 (s, 1H), 6.97 (d, J=8.4 Hz, 2H), 3.87 (dd, J=23.6, 10.4 Hz, 4H), 3.73 (dt, J=29.4, 7.6 Hz, 3H), 3.60 (dd, J=15.2, 6.8 Hz, 1H), 3.48 (d, J=10.8 Hz, 2H), 3.11-2.92 (m, 4H), 2.45-2.18 (m, 8H), 2.03 (d, J=5.2 Hz, 1H), 1.81 (dt, J=19.6, 9.2 Hz, 2H).

### Example 32: Synthesis of (S)-(3-aminopyrrolidin-1-yl)(5-(4-(4-isopropylpiperazin-1-yl)phenyl)-3-methylthiophen-2-yl)methanone Compound 159)

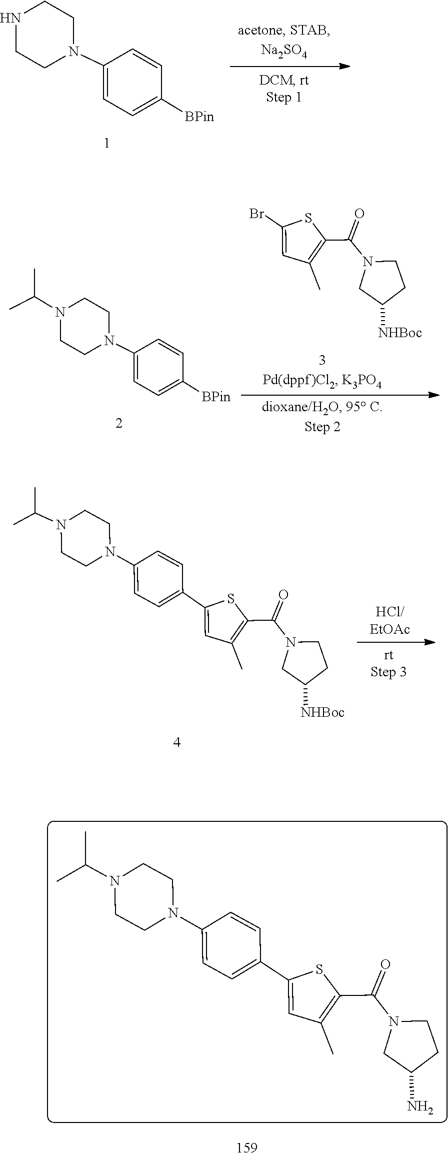

### Step 1 1-isopropyl-4-(4-(4,4,5,5-tetramethyl-1,3,2-dioxaborolan-2-yl)phenyl)piperazine

To a mixture of 1-(4-(4,4,5,5-tetramethyl-1,3,2-dioxaborolan-2-yl)phenyl)piperazine (400 mg, 1.39 mmol) in DCM (40 mL) were added STAB (2.06 g, 9.72 mmol), acetone (322 mg, 5.56 mmol) and Na₂SO₄ (118 mg, 0.83 mmol), then it was stirred at 25° C. for 12 hours. LCMS showed the reaction was completed. The reaction mixture was concentrated to dryness. EtOAc (20 mL*3) was added and the mixture was concentrated to dryness. The mixture was diluted with sat. NaHCO₃ solution (50 mL) and then extracted with EtOAc (30 mL*3). The combined organic layers were washed with brine (100 mL), dried over Na₂SO₄ and concentrated to dryness. The crude product was purified by column chromatography on silica gel (MeOH/DCM=7%) to give the desired product (200 mg, 39.3% yield) as a yellow solid.

### Step 2 tert-butyl (S)-(1-(5-(4-(4-isopropylpiperazin-1-yl)phenyl)-3-methylthiophene-2-carbonyl)pyrrolidin-3-yl)carbamate

To a mixture of compound 2 (200 mg, 0.61 mmol) and compound 3 (189 mg, 0.48 mmol) in 1,4-dioxane (6 mL) and H₂O (1 mL) were added K₃PO₄ (386 mg, 1.82 mmol) and Pd(dppf)Cl₂ (99 mg, 0.12 mmol), then it was stirred at 95° C. under N₂ for 16 hours. LCMS showed the reaction was completed. The reaction mixture was concentrated to dryness. The crude product was purified by column chromatography on silica gel (EtOAc/PE=92%) to afford desired product (124 mg, 36.0% yield) as a yellow solid.

### Step 3 (S)-(3-aminopyrrolidin-1-yl)(5-(4-(4-isopropylpiperazin-1-yl)phenyl)-3-methylthiophen-2-yl)methanone

A mixture of compound 4 (124 mg, 0.24 mmol) in HCl/EtOAc (2 mL) was stirred at 25° C. for 3 hours. LCMS showed the reaction was complete. The reaction mixture was concentrated to dryness. The crude product was purified by prep-HPLC to afford desired product (Compound 159; 14.5 mg, 14.2% yield) as a white solid. MS (ESI): mass calc'd. for C₂₃H₃₂N₄OS 412.23, m/z found 413.2 [M+H]⁺. ¹H NMR (400 MHz, MeOD) δ 7.52 (d, J=8.8 Hz, 2H), 7.08 (s, 1H), 6.99 (d, J=8.8 Hz, 2H), 3.83-3.73 (m, 2H), 3.69-3.51 (m, 2H), 3.38-3.33 (m, 1H), 3.30-3.24 (m, 4H), 2.79-2.69 (m, 5H), 2.33 (s, 3H), 2.24-2.12 (m, 1H), 1.91-1.73 (m, 1H), 1.14 (d, J=6.4 Hz, 6H).

### Example 33: Synthesis of (S)-(3-aminopyrrolidin-1-yl)(3-methyl-5-(4-(piperazin-1-yl)phenyl)thiophen-2-yl)methanone (Compound 162)

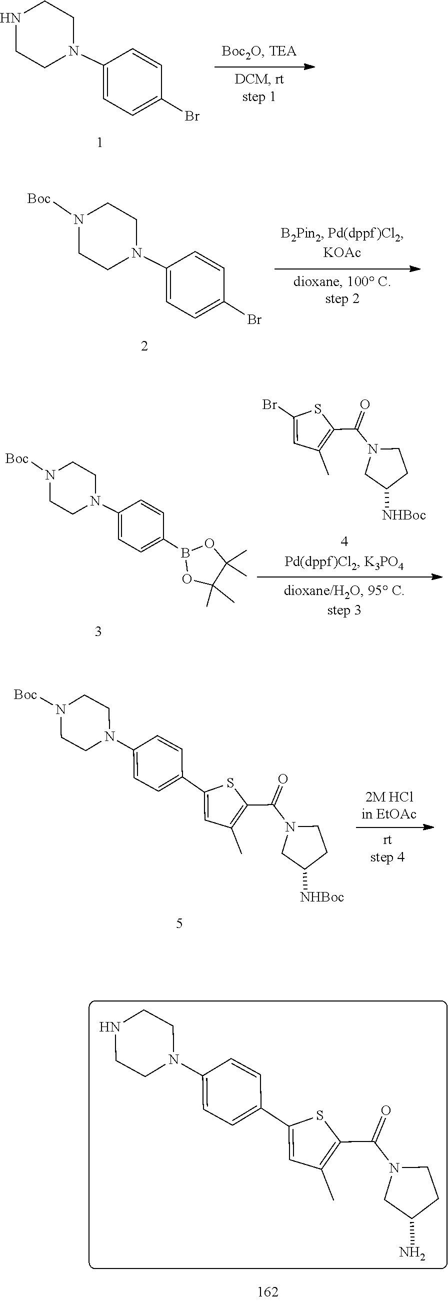 [SMILES: Brc1ccc(N2CCNCC2)cc1 ; CC(C)(C)OC(=O)N1CCN(c2ccc(Br)cc2)CC1 ; CC(C)(C)OC(=O)N1CCN(c2ccc(B3OC(C)(C)C(C)(C)O3)cc2)CC1 ; Cc1cc(Br)sc1C(=O)N1CC[C@H](NC(=O)OC(C)(C)C)C1 ; Cc1cc(-c2ccc(N3CCN(C(=O)OC(C)(C)C)CC3)cc2)sc1C(=O)N1CC[C@H](NC(=O)OC(C)(C)C)C1 ; Cc1cc(-c2ccc(N3CCNCC3)cc2)sc1C(=O)N1CC[C@H](N)C1]

### Step 1 Synthesis of tert-butyl 4-(4-bromophenyl)piperazine-1-carboxylate

To a solution of 1-(4-bromophenyl)piperazine (5 g, 20.7 mmol) in 60 mL DCM were added Boc₂O (5.42 g, 24.8 mmol) and TEA (3.52 g, 34.7 mmol), the mixture was stirred at 25° C. for 2 hours. After the reaction, the solvent was removed under reduced pressure, the residue was purified by column chromatography to afford the tert-butyl 4-(4-bromophenyl)piperazine-1-carboxylate (7.043 g, yield=98.55%) as an off-white solid. MS (ESI): mass calc'd. for C15H22BrN2O2 341.09, found 341.0 [M+H]⁺

### Step 2 Synthesis of tert-butyl 4-(4-(4,4,5,5-tetramethyl-1,3,2-dioxaborolan-2-yl)phenyl)piperazine-1-carboxylate

To a solution of tert-butyl 4-(4-bromophenyl)piperazine-1-carboxylate (7 g, 20.5 mmol) and B₂Pin₂ (6.25 g, 24.6 mmol) in 50 mL 1,4-dioxane were added Pd(dppf)Cl₂DCM (3.35 g, 4.1 mmol) and KOAc (6.04 g, 61.5 mmol), the mixture was stirred at 100° C. under N₂ for 16 hours. After the reaction, the solvent was removed under reduced pressure, the residue was purified by column chromatography to afford 4-(4-(4,4,5,5-tetramethyl-1,3,2-dioxaborolan-2-yl)phenyl)piperazin-1-yl pivalate (8 g, 98.05% yield) as a yellow solid. MS (ESI): mass calc'd. for C21H34BN2O4 389.26, found 389.2 [M+H]⁺

### Step 3 Synthesis of tert-butyl (S)-4-(4-(5-(3-((tert-butoxycarbonyl)amino)pyrrolidine-1-carbonyl)-4-methylthiophen-2-yl)phenyl)piperazine-1-carboxylate

To a solution of tert-butyl (S)-(1-(5-bromo-3-methylthiophene-2-carbonyl)pyrrolidin-3-yl)carbamate (200 mg, 0.5137 mmol) and tert-butyl 4-(4-(4,4,5,5-tetramethyl-1,3,2-dioxaborolan-2-yl)phenyl)piperazine-1-carboxylate (171.0 mg, 0.6164 mmol) in 9 mL dioxane/H₂O (v:v=8:1) were added Pd(dppf)Cl₂DCM (83.9 mg, 0.1027 mmol) and K₃PO₄ (327.1 mg, 1.5411 mmol), the mixture was stirred at 95° C. under N₂ for 16 hours. After the reaction, the solvent was removed under reduced pressure, the residue was purified by column chromatography to afford tert-butyl (S)-4-(4-(5-(3-((tert-butoxycarbonyl)amino)pyrrolidine-1-carbonyl)-4-methylthiophen-2-yl)phenyl)piperazine-1-carboxylate (150 mg, 50.55% yield) as a yellow solid. MS (ESI): mass calc'd. for C30H43N4O5S 571.30, found 571.3 [M+H]⁺

### Step 4 Synthesis of (S)-(3-aminopyrrolidin-1-yl)(3-methyl-5-(4-(piperazin-1-yl)phenyl)thiophen-2-yl)methanone

To a solution of tert-butyl (S)-4-(4-(5-(3-((tert-butoxycarbonyl)amino)pyrrolidine-1-carbonyl)-4-methylthiophen-2-yl)phenyl)piperazine-1-carboxylate (150 mg, 0.2624 mmol) in 2 mL EtOAc was added HCl (4 mL, 2M in EtOAc), the mixture was stirred at 25° C. for 12 hours. After the reaction, the mixture was filtered, the filter cake was washed with EtOAc for several times to afford (S)-(3-aminopyrrolidin-1-yl)(3-methyl-5-(4-(piperazin-1-yl)phenyl)thiophen-2-yl)methanone (Compound 162; 64.5 mg, 63.03% yield) as a yellow solid. MS (ESI): mass calc'd. for C20H27N4OS 371.19, found 371.1 [M+H]⁺. ¹H NMR (400 MHz, MeOD) δ 7.62 (d, J=8.2 Hz, 2H), 7.22-7.19 (m, 3H), 4.02-4.00 (m, 2H), 3.89-3.83 (m, 1H), 3.80-3.72 (m, 2H), 3.62-3.59 (m, 4H), 3.49-3.46 (m, 4H), 2.51-2.45 (m, 1H), 2.35 (s, 3H), 2.19-2.16 (m, 1H).

### Example 35: Synthesis of (S)-(3-aminopyrrolidin-1-yl)(5-(isochroman-6-yl)-3-methylthiophen-2-yl)methanone (Compound 165)

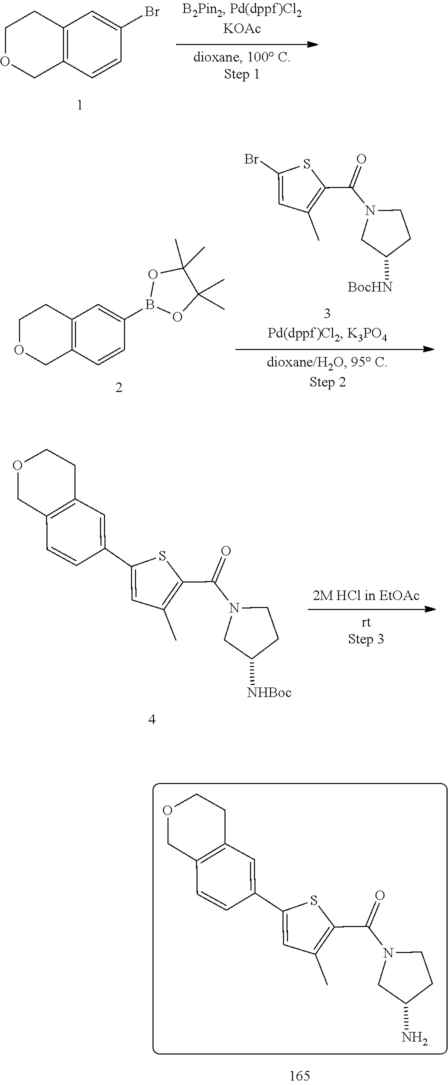 [SMILES: Brc1ccc2c(c1)CCOC2 ; CC1(C)OB(c2ccc3c(c2)CCOC3)OC1(C)C ; Cc1cc(Br)sc1C(=O)N1CC[C@H](NC(=O)OC(C)(C)C)C1 ; Cc1cc(-c2ccc3c(c2)CCOC3)sc1C(=O)N1CC[C@H](NC(=O)OC(C)(C)C)C1 ; Cc1cc(-c2ccc3c(c2)CCOC3)sc1C(=O)N1CC[C@H](N)C1]

### Step 1 2-(isochroman-6-yl)-4,4,5,5-tetramethyl-1,3,2-dioxaborolane

To a solution of 6-bromo-3,4-dihydro-1H-2-benzopyran (250 mg, 1.17 mmol) in dioxane (10 mL) were added B₂Pin₂ (298 mg, 1.17 mmol), KOAc (345.4 mg, 3.52 mmol) and Pd(dppf)Cl₂DCM (191.5 mg, 0.23 mmol). The mixture was stirred at 100° C. for 16 h. After the reaction, the mixture was filtered and concentrated under reduced pressure. The residue was purified by flash chromatography using EtOAc:PE=1:8 as eluent to give 2-(isochroman-6-yl)-4,4,5,5-tetramethyl-1,3,2-dioxaborolane (200 mg, 62.25% yield) as a yellow oil. LCMS (ESI) calc'd for C15H22BO3+ [M+H]+ m/z 261.17, found 261.

### Step 2 tert-butyl (S)-(1-(5-(isochroman-6-yl)-3-methylthiophene-2-carbonyl)pyrrolidin-3-yl)carbamate

To a solution of tert-butyl (S)-(1-(5-bromo-3-methylthiophene-2-carbonyl)pyrrolidin-3-yl)carbamate (300 mg, 0.77 mmol) in dioxane/H₂O=8:1 (18 mL) were added 2-(isochroman-6-yl)-4,4,5,5-tetramethyl-1,3,2-dioxaborolane (200 mg, 0.77 mmol), K₃PO₄ (490.7 mg, 2.31 mmol) and Pd(dppf)Cl₂DCM (125.8 mg, 0.15 mmol). The mixture was stirred at 95° C. for 16 h. After the reaction, the mixture was filtered and concentrated under reduced pressure. The residue was purified by flash chromatography (petroleum ether/ethyl acetate=1/1) to give tert-butyl (S)-(1-(5-(isochroman-6-yl)-3-methylthiophene-2-carbonyl)pyrrolidin-3-yl)carbamate (200 mg, 55.7% yield) as a yellow oil. LCMS (ESI) calc'd for C24H31N2O4S+ [M+H]+ m/z 443.20, found 443.

### Step 4 (S)-(3-aminopyrrolidin-1-yl)(5-(isochroman-6-yl)-3-methylthiophen-2-yl)methanone

To the solution of tert-butyl (S)-(1-(5-(isochroman-6-yl)-3-methylthiophene-2-carbonyl)pyrrolidin-3-yl)carbamate (200 mg, 0.45 mmol) in 4 mL EtOAc was added HCl (3 mL, 2M in EtOAc). The mixture was stirred at rt for 16 h. The LCMS showed the reaction was completed and the desired product was found. The mixture was concentrated under reduced pressure. The residue was purified by prep-HPLC to give (S)-(3-aminopyrrolidin-1-yl)(5-(isochroman-6-yl)-3-methylthiophen-2-yl)methanone (Compound 165; 88.6 mg, 54.4% Yield) as a yellow solid. LCMS (ESI) calc'd for C19H23N2O2S+ [M+H]+ m/z 343.15, found 343. ¹H NMR (400 MHz, MeOD) δ 7.46-7.39 (m, 2H), 7.23 (s, 1H), 7.04 (t, J=8.4 Hz, 1H), 4.75 (s, 2H), 4.00 (d, J=8.3 Hz, 2H), 3.99 (s, 2H), 3.88-3.76 (m, 2H), 3.75-3.62 (m, 2H), 2.87 (t, J=5.7 Hz, 2H), 2.52-2.40 (m, 1H), 2.36 (s, 3H), 2.21-2.09 (m, 1H).

### Example 36: Synthesis of (S)-(3-aminopyrrolidin-1-yl)(5-(4-(4-(dimethylamino)tetrahydro-2H-pyran-4-yl)phenyl)-3-methylthiophen-2-yl)methanone (Compound 166)

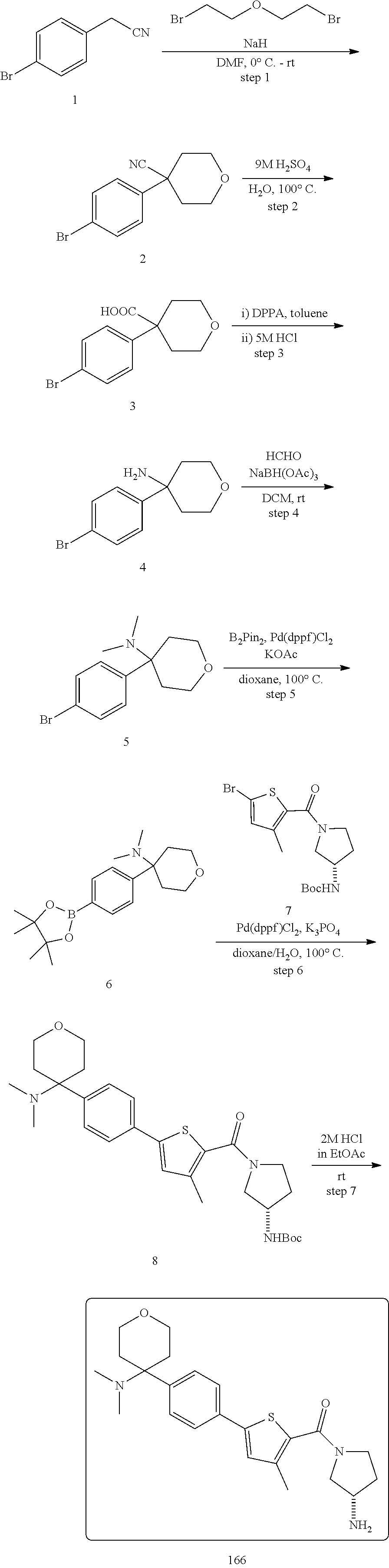

### Step 1 4-(4-bromophenyl) tetrahydro-2H-pyran-4-carbonitrile

The NaH (2.04 g, 60% in oil) was added in portions to a solution of 2-(4-bromophenyl) acetonitrile (4 g, 0.0204 mol) in dry DMF (40 mL) and stirred at 0° C. for 1 h. Then 1-bromo-2-(2-bromoethoxy) ethane (4.73 g, 0.0204 mol) was added to the above solution at 0° C. and stirred at room temperature for overnight. The LCMS showed the reaction was completed and the desired MS was found. After completion of the reaction, the mixture was quenched by addition of water, then extracted with ethyl acetate. The combined organic layer was washed with water and saturated NaCl solution, dried over anhydrous Na₂SO₄ and evaporated in vacuum. The residue was purified by flash chromatography (PE/EA=90/10) to give 4-(4-bromophenyl) oxane-4-carbonitrile (4.4 g, 73.04% yield) as a yellow solid.

### Step 2 4-(4-bromophenyl)tetrahydro-2H-pyran-4-carboxylic acid

A solution of 9 M H₂SO₄ was added into 4-(4-bromophenyl) oxane-4-carbonitrile (4 g, 1 eq) and was refluxed at 100° C. for overnight. After completion of the reaction, the mixture was diluted with water and then extracted with ethyl acetate. The combined organic layer was washed with water and saturated NaCl solution, dried over anhydrous Na₂SO₄ and evaporated in vacuum. Purification of the crude product by silica gel column chromatography to give the desired product (4 g, 84.00% yield) as a yellow solid.

### Step 3 4-(4-bromophenyl) tetrahydro-2H-pyran-4-amine

4-(4-bromophenyl) oxane-4-carboxylic acid (4 g, 1 eq) was added into a three-neck round-bottom flask under N₂, Toluene (56 mL) and TEA (3.43 g, 2.2 eq) were then added via a syringe. DPPA (4.66 g, 1.1 eq) was added via a syringe and the mixture was stirred at 90° C. under N₂ for 2 h. After completion of the reaction, the mixture was cooled to the room temperature and diluted with EtOAc. The combined organic layer was washed with NaHCO₃ solution and brine, dried over anhydrous Na₂SO₄ and evaporated in vacuum. Then 5 M HCl solution (11 mL) was added and the mixture was refluxed at 100° C. for 5 h. After the mixture was cooled to the room temperature, toluene was added and evaporated in vacuum. The obtained solid was dissolved into the saturated NaHCO₃ solution (300 mL) and EtOAc (300 mL). The organic layer was separated, which was washed with brine, dried over anhydrous Na₂SO₄ and evaporated in vacuum. Purification of the crude product by silica gel column chromatography to give the desired product (2.5 g, 57.14% yield) as a yellow solid.

### Step 4 4-(4-bromophenyl)-N,N-dimethyltetrahydro-2H-pyran-4-amine

To the solution of 4-(4-bromophenyl)oxan-4-amine (1 g, 0.0039 mol) in DCM (50 mL) was added 37% HCHO (0.95 g, 0.0117 mol), Na₂SO₄ (0.33 g, 0.0023 mol) and Sodium triacetoxyborohydride (4.96 g, 0.0234 mol). The mixture was stirred at rt for 16 h. The LCMS showed the reaction was completed and the desired MS was found. 50 mL NaHCO₃ solution was added to the reaction mixture, which was extracted with EtOAc (3*50 mL). Combined with organic layers, washed with brine, dried over anhydrous Na₂SO₄ and evaporated in vacuum. Purification of the crude product by silica gel column chromatography to give the desired product (1.1 g, 89.74% yield) as a yellow solid.

### Step 5N,N-dimethyl-4-(4-(4,4,5,5-tetramethyl-1,3,2-dioxaborolan-2-yl)phenyl)tetrahydro-2H-pyran-4-amine

To the solution of 4-(4-bromophenyl)-N,N-dimethyloxan-4-amine (400 mg, 1.4075 mmol) in 1,4-dioxane (8 mL) were added B₂Pin₂ (393.16 mg, 1.5482 mmol), Pd(dppf)Cl₂DCM (229.7 mg, 0.2815 mmol) and KOAc (414.4 mg, 4.2225 mmol). The mixture was stirred at 95° C. under N₂ for 16 h. The LCMS showed the reaction was completed and the desired product was found. After cooled to rt, the mixture was filtered and concentrated under reduced pressure. The residue was purified by silica gel column chromatography to give the desired product (300 mg, 57.91% yield) as a yellow solid.

### Step 6 tert-butyl(S)-(1-(5-(4-(4-(dimethylamino)tetrahydro-2H-pyran-4-yl)phenyl)-3-methylthiophene-2-carbonyl)pyrrolidin-3-yl)carbamate

To the solution of N,N-dimethyl-4-(4-(4,4,5,5-tetramethyl-1,3,2-dioxaborolan-2-yl)phenyl)tetrahydro-2H-pyran-4-amine (300 mg, 0.9056 mmol) in dioxane/H₂O=8:1 (9 mL) were added tert-butyl N-((3S)-1-((5-bromo-3-methylthiophen-2-yl)carbonyl)pyrrolidin-3-yl)carbamate (352.56 mg, 0.9056 mmol), K₃PO₄ (576.70 mg, 2.7168 mmol) and Pd(dppf)Cl₂DCM (147.79 mg, 0.1811 mmol). The mixture was stirred at 95° C. under N₂ for 16 h. The LCMS showed the reaction was completed and the desired product was found. The solvent was removed under reduced pressure and the residue was purified by silica gel column chromatography (EtOAc:MeOH=10:1) to give the desired product (120 mg, 23.21% yield) as a yellow solid.

### Step 7 (S)-(3-aminopyrrolidin-1-yl)(5-(4-(4-(dimethylamino)tetrahydro-2H-pyran-4-yl)phenyl)-3-methylthiophen-2-yl)methanone

To the solution of tert-butyl(S)-(1-(5-(4-(4-(dimethylamino)tetrahydro-2H-pyran-4-yl)phenyl)-3-methylthiophene-2-carbonyl)pyrrolidin-3-yl)carbamate (120 mg, 0.2336 mmol) in EtOAc (4 mL) was added 2M HCl in EA (5 mL). The mixture was stirred at rt for 16 h. The LCMS showed the reaction was completed and the desired MS was found. The mixture was filtered and concentrated under reduced pressure to give the desired product (Compound 166; 60 mg, 58.99% yield) as a yellow solid. MS (ESI): mass calc'd for C23H32N3O2S 414, m/z found 414 [M+H]⁺. ¹H NMR (400 MHz, MeOD) δ 7.89 (d, J=7.6 Hz, 2H), 7.75 (d, J=7.6 Hz, 2H), 7.43 (s, 1H), 4.01 (d, J=8.8 Hz, 4H), 3.91-3.68 (m, 3H), 3.25 (t, J=11.6 Hz, 2H), 2.98 (d, J=12.8 Hz, 2H), 2.67 (s, 6H), 2.52-2.42 (m, 1H), 2.38 (s, 3H), 2.19 (t, J=10.8 Hz, 3H).

### Example 34: Synthesis of (S)-(3-aminopyrrolidin-1-yl)(3-methyl-5-(4-(1-(oxetan-3-yl)piperidin-4-yl)phenyl)thiophen-2-yl)methanone (Compound 164)

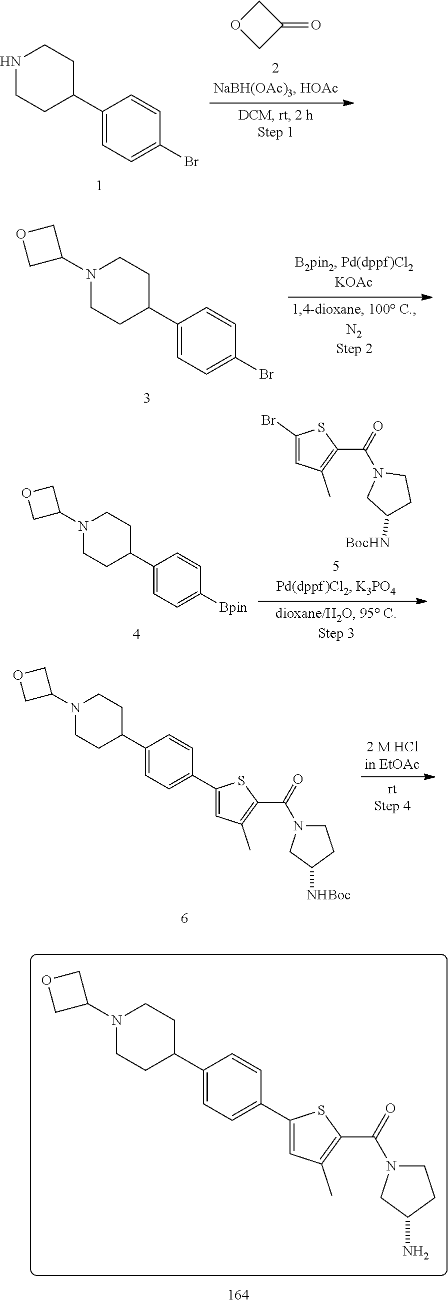

### Step 1 4-(4-bromophenyl)-1-(oxetan-3-yl)piperidine

To a solution of 4-(4-bromophenyl)piperidine (240 mg, 1 mmol) in DCM (10 mL) was added oxetan-3-one (72 mg, 1 mmol), NaBH(OAc)₃ (317.7 mg, 1.5 mmol) and HOAc (60 mg, 1 mmol). The mixture was stirred at rt for 3 h. The LCMS showed the reaction was completed and the desired product was found. The resulting mixture was washed with H₂O (20 mL). The combined organic layer was dried over Na₂SO₄, filtered and concentrated under reduced pressure to give 4-(4-bromophenyl)-1-cyclobutylpiperidine (290 mg, 93.07% yield) as a yellow solid.

### Step 2 1-(oxetan-3-yl)-4-(4-(4,4,5,5-tetramethyl-1,3,2-dioxaborolan-2-yl)phenyl)piperidine

To a solution of 4-(4-bromophenyl)-1-(oxetan-3-yl)piperidine (290 mg, 0.98 mmol) in dioxane (10 mL) was added 4,4,5,5-tetramethyl-2-(4,4,5,5-tetramethyl-1,3,2-dioxaborolan-2-yl)-1,3,2-dioxaborolane (248 mg, 0.98 mmol), Potassium acetate (288 mg, 3 mmol) and PdCl2(dppf)(160 mg, 0.19 mmol). The mixture was stirred at 95° C. for 16 h. After cooling rt, the mixture was filtered and concentrated under reduced pressure. The residue was purified by flash chromatography using EtOAc as eluent to give 1-(oxetan-3-yl)-4-(4-(4,4,5,5-tetramethyl-1,3,2-dioxaborolan-2-yl)phenyl)piperidine (258 mg, 73% yield) as a yellow oil.

### Step 3 tert-butyl (S)-(1-(3-methyl-5-(4-(1-(oxetan-3-yl)piperidin-4-yl)phenyl)thiophene-2-carbonyl)pyrrolidin-3-yl)carbamate

To a solution of tert-butyl (S)-(1-(5-bromo-3-methylthiophene-2-carbonyl)pyrrolidin-3-yl)carbamate (280 mg, 0.72 mmol) in dioxane/H₂O=8:1 (18 mL) were added 1-(oxetan-3-yl)-4-(4-(4,4,5,5-tetramethyl-1,3,2-dioxaborolan-2-yl)phenyl)piperidine (247 mg, 0.72 mmol), K₃PO₄ (458 mg, 2.15 mmol) and Pd(dppf)Cl₂DCM (117.4 mg, 0.14 mmol). The mixture was stirred at 95° C. for 16 h. After the reaction, the mixture was filtered and concentrated under reduced pressure. The residue was purified by flash chromatography (petroleum ether/ethyl acetate=1/8) to give tert-butyl (S)-(1-(3-methyl-5-(4-(1-(oxetan-3-yl)piperidin-4-yl)phenyl)thiophene-2-carbonyl)pyrrolidin-3-yl)carbamate (190 mg, 47.7% yield) as a yellow oil. LCMS (ESI) calc'd for C29H40N3O4S+ [M+H]+ m/z 526.28, found 526.

### Step 4 (S)-(3-aminopyrrolidin-1-yl)(3-methyl-5-(4-(1-(oxetan-3-yl)piperidin-4-yl)phenyl)thiophen-2-yl)methanone

To the solution of tert-butyl (S)-(1-(3-methyl-5-(4-(1-(oxetan-3-yl)piperidin-4-yl)phenyl)thiophene-2-carbonyl)pyrrolidin-3-yl)carbamate (190 mg, 0.36 mmol) in 4 mL EtOAc was added HCl (3 mL, 2M in EtOAc). The mixture was stirred at rt for 16 h. The LCMS showed the reaction was completed and the desired product was found. The mixture was concentrated under reduced pressure. The residue was purified by prep-HPLC to give (S)-(3-aminopyrrolidin-1-yl)(3-methyl-5-(4-(1-(oxetan-3-yl)piperidin-4-yl)phenyl)thiophen-2-yl)methanone (Compound 164; 20 mg, 11% yield) as a white solid. LCMS (ESI) calc'd for C24H31N3O2S+ [M+H]+ m/z 426.22, found 426. ¹H NMR (400 MHz, MeOD) δ 7.63 (d, J=8.3 Hz, 2H), 7.34 (d, J=8.2 Hz, 2H), 7.25 (s, 1H), 4.87 (dd, J=11.9, 6.0 Hz, 3H), 4.43 (s, 1H), 4.02-3.94 (m, 2H), 3.81 (tdd, J=11.8, 8.9, 5.1 Hz, 2H), 3.69 (dd, J=15.0, 6.7 Hz, 1H), 3.61 (d, J=9.6 Hz, 2H), 3.11-2.89 (m, 3H), 2.44 (td, J=13.9, 7.6 Hz, 1H), 2.37 (d, J=8.5 Hz, 3H), 2.14 (td, J=13.2, 6.1 Hz, 3H), 2.07-1.97 (m, 2H).

### Example 37: Synthesis of (S)-(3-aminopyrrolidin-1-yl)(3-methyl-5-(4-(1-(tetrahydro-2H-thiopyran-4-yl)piperidin-4-yl)phenyl)thiophen-2-yl)methanone (Compound 167)

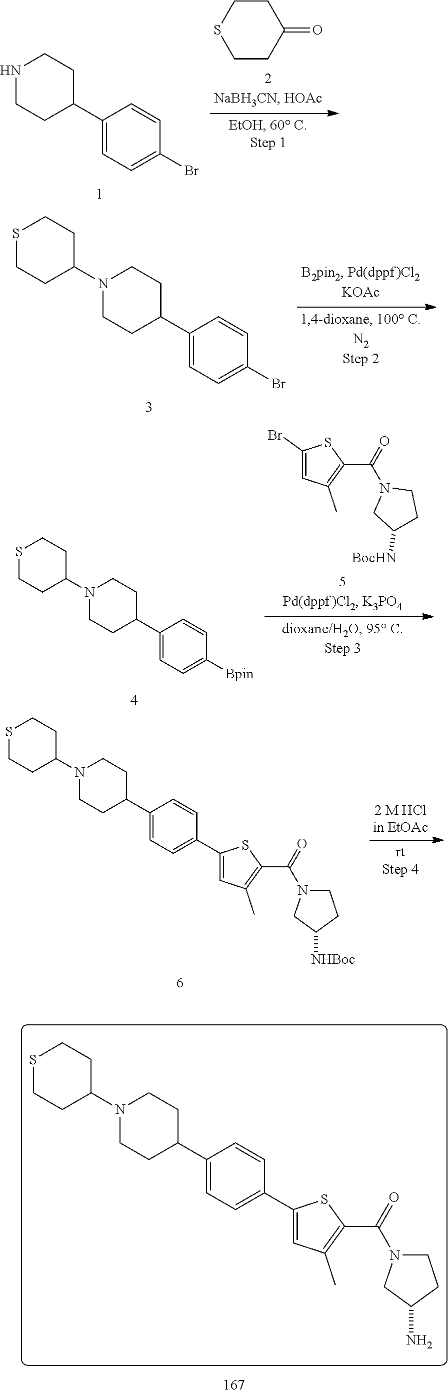

### Step 1 4-(4-bromophenyl)-1-(tetrahydro-2H-thiopyran-4-yl)piperidine

To a solution of 4-(4-bromophenyl)piperidine (1.0 g, 4.2 mmol) and thian-4-one (0.73 g, 6.3 mmol) in EtOH (6 mL) was added NaBH₃CN (0.32 g, 5 mmol), followed by HOAc (0.5 g, 8.4 mmol), the mixture was stirred at 25° C. for 12 h. After the reaction, NaOH (30 mL, 1N in H₂O) was added and stirred for 30 min, the mixture was extracted with EtOAc for 3 times. Combined with EtOAc phases, washed with brine, dried over Na₂SO₄ and filtered, the solvent was removed under reduced pressure, the residue was purified by column chromatography to afford 4-(4-bromophenyl)-1-(thian-4-yl)piperidine (1.5 g crude, 95% yield) as yellowish solid.

### Step 2 1-(tetrahydro-2H-thiopyran-4-yl)-4-(4-(4,4,5,5-tetramethyl-1,3,2-dioxaborolan-2-yl)phenyl)piperidine

To a mixture of compound 3 (300 mg, 0.88 mmol) in 1,4-dioxane (7 mL) were added B₂Pin₂ (246 mg, 0.97 mmol), KOAc (260 mg, 2.64 mmol) and Pd(dppf)Cl₂DCM (719 mg, 0.88 mmol), then it was stirred at 100° C. under N₂ for 12 hours. The reaction mixture was concentrated to dryness. The crude product was purified by column chromatography on silica gel (petroleum ether/EtOAc=2/1) to afford desired product (180 mg, 47.4% yield) as a black solid.

### Step 3 tert-butyl (S)-(1-(3-methyl-5-(4-(1-(tetrahydro-2H-thiopyran-4-yl)piperidin-4-yl)phenyl)thiophene-2-carbonyl)pyrrolidin-3-yl)carbamate

To a mixture of compound 4 (179 mg, 0.46 mmol) in 1,4-dioxane (4 mL) and H₂O (0.5 mL) were added compound 5 (150 mg, 0.39 mmol), K₃PO₄ (245 mg, 1.16 mmol) and Pd(dppf)Cl₂DCM (314 mg, 0.39 mmol), then the mixture was stirred at 95° C. under N₂ for 12 hours. LCMS showed the reaction was completed. The reaction mixture was concentrated to dryness. The crude product was purified by column chromatography on silica gel (EtOAc/PE=1/1) to afford desired product (150 mg, 61.5% yield) as a black solid.

### Step 4 (S)-(3-aminopyrrolidin-1-yl)(3-methyl-5-(4-(1-(tetrahydro-2H-thiopyran-4-yl)piperidin-4-yl)phenyl)thiophen-2-yl)methanone

A mixture of compound 6 (150 mg, 0.26 mmol) in HCl/EtOAc (3 mL) was stirred at 25° C. for 2 hours. LCMS showed the reaction was completed. The reaction mixture was concentrated to dryness. The crude product was purified by prep-HPLC to give the desired product (Compound 167; 21.7 mg, 16.9% yield) as a white solid. LCMS (ESI): mass calc'd. for C₂₆H₃₆N₃OS₂ 470.23, m/z found 470.3 [M+H]⁺. ¹H NMR (400 MHz, MeOD) δ 8.52 (s, 1.5H), 7.61 (d, J=8.0 Hz, 2H), 7.32 (d, J=8.0 Hz, 2H), 7.24 (s, 1H), 4.02-3.89 (m, 2H), 3.88-3.71 (m, 2H), 3.69-3.61 (m, 1H), 3.51 (d, J=12.0 Hz, 2H), 3.27-3.15 (m, 3H), 2.92-2.74 (m, 5H), 2.48-2.38 (m, 3H), 2.35 (s, 3H), 2.16-2.02 (m, 5H), 1.97-1.85 (m, 2H).

### Example 38: Synthesis of (S)-(3-aminopyrrolidin-1-yl)(5-(4-(1-ethylpiperidin-4-yl)phenyl)-3-methylthiophen-2-yl)methanone (Compound 176)

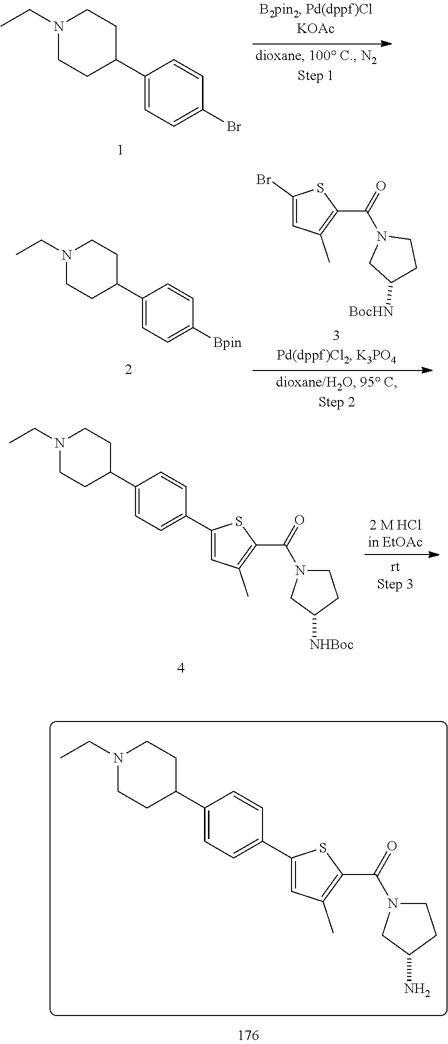

### Step 1 Synthesis of 1-ethyl-4-(4-(4,4,5,5-tetramethyl-1,3,2-dioxaborolan-2-yl)phenyl)piperidine

To a solution of 4-(4-bromophenyl)-1-ethylpiperidine (220 mg, 0.82 mmol) in dioxane (10 mL) were added B₂Pin₂ (208.3 mg, 0.82 mmol), KOAc (241.5 mg, 2.46 mmol) and Pd(dppf)Cl₂DCM (133.9 mg, 0.16 mmol). The mixture was stirred at 100° C. for 16 h. After the reaction, the mixture was filtered and concentrated under reduced pressure. The residue was purified by flash chromatography EA:MeOH=9:1 to give 1-ethyl-4-(4-(4,4,5,5-tetramethyl-1,3,2-dioxaborolan-2-yl)phenyl)piperidine (180 mg, 66.1% yield) as a yellow solid.

### Step 2 Synthesis of tert-butyl (S)-(1-(5-(4-(1-ethylpiperidin-4-yl)phenyl)-3-methylthiophene-2-carbonyl)pyrrolidin-3-yl)carbamate

To a solution of tert-butyl (S)-(1-(5-bromo-3-methylthiophene-2-carbonyl)pyrrolidin-3-yl)carbamate (220 mg, 0.56 mmol) in dioxane/H₂O=8:1 (18 mL) were added 1-ethyl-4-(4-(4,4,5,5-tetramethyl-1,3,2-dioxaborolan-2-yl)phenyl)piperidine (178 mg, 0.56 mmol), K₃PO₄ (360 mg, 1.7 mmol) and Pd(dppf)Cl₂DCM (92.2 mg, 0.11 mmol). The mixture was stirred at 95° C. under N₂ for 16 h. After the reaction, the mixture was filtered and concentrated under reduced pressure. The residue was purified by flash chromatography (MeOH/ethyl acetate=1/9) to give tert-butyl (S)-(1-(5-(4-(1-ethylpiperidin-4-yl)phenyl)-3-methylthiophene-2-carbonyl)pyrrolidin-3-yl)carbamate (185 mg, 59.2% yield) as a yellow solid. LCMS (ESI) calc'd for C28H40N3O3S+ [M+H]+ m/z 498.28, found 498.

### Step 3 Synthesis of (S)-(3-aminopyrrolidin-1-yl)(5-(4-(1-ethylpiperidin-4-yl)phenyl)-3-methylthiophen-2-yl)methanone

To the solution of tert-butyl (S)-(1-(5-(4-(1-ethylpiperidin-4-yl)phenyl)-3-methylthiophene-2-carbonyl)pyrrolidin-3-yl)carbamate (185 mg, 0.37 mmol) in 4 mL EtOAc was added HCl (3 mL, 2M in EtOAc). The mixture was stirred at rt for 16 h. The LCMS showed the reaction was completed and the desired MS was found. The mixture was concentrated under reduced pressure. The residue was purified by prep-HPLC to give the (S)-(3-aminopyrrolidin-1-yl)(5-(4-(1-ethylpiperidin-4-yl)phenyl)-3-methylthiophen-2-yl)methanone (Compound 176; 7.6 mg, 5% Yield) as a white solid. LCMS (ESI) calc'd for C23H32N3OS+ [M+H]+ m/z 398.2, found 398. ¹H NMR (400 MHz, MeOD) δ 7.58 (d, J=8.3 Hz, 2H), 7.31 (d, J=8.3 Hz, 2H), 7.21 (s, 1H), 3.79 (dt, J=11.7, 7.0 Hz, 2H), 3.69-3.55 (m, 2H), 3.38-3.34 (m, 1H), 3.14 (d, J=11.7 Hz, 2H), 2.62 (tt, J=11.9, 4.0 Hz, 1H), 2.53 (q, J=7.3 Hz, 2H), 2.34 (s, 3H), 2.22-2.18 (m, 1H), 2.15 (dd, J=11.8, 2.6 Hz, 2H), 1.89 (t, J=9.0 Hz, 2H), 1.86-1.80 (m, 2H), 1.80 (s, 1H), 1.17 (t, J=7.2 Hz, 3H).

### Example 39: Synthesis of (S)-(3-aminopyrrolidin-1-yl)(3-methyl-5-(4-(1-(tetrahydro-2H-pyran-4-yl)piperidin-4-yl)phenyl)thiophen-2-yl)methanone (Compound 178)

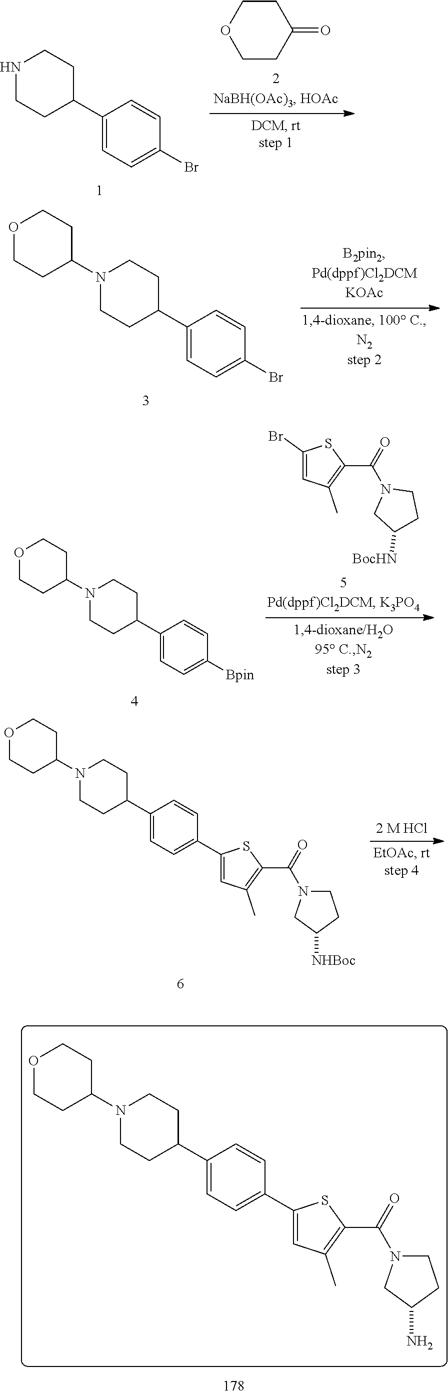

### Step 1 Synthesis of 4-(4-bromophenyl)-1-(tetrahydro-2H-pyran-4-yl)piperidine

To a solution of 4-(4-bromophenyl)piperidine (500 mg, 2.0821 mmol) and oxan-4-one (208.5 mg, 2.0821 mmol) in DCM (5 mL) were added NaBH₃CN (661.9 mg, 3.1231 mmol) and HOAc (125.0 mg, 2.0821 mmol), the mixture was stirred at 25° C. for 12 h. After the reaction, NaOH (15 mL, 1N in H₂O) was added and stirred for 30 min, the mixture was extracted with EtOAc for 3 times. Combined with EtOAc phases, washed with brine, dried over Na₂SO₄ and filtered. The solvent was removed under reduced pressure, the residue was purified by column chromatography to afford 382 mg 4-(4-bromophenyl)-1-(oxan-4-yl)piperidine as yellowish solid.

MS (ESI): mass calc'd. for C16H23BrNO 324.10, found 324 [M+1]⁺

### Step 2 Synthesis of 1-(oxan-4-yl)-4-(4-(4,4,5,5-tetramethyl-1,3,2-dioxaborolan-2-yl)phenyl)piperidine

To a solution of 4-(4-bromophenyl)-1-(oxan-4-yl)piperidine (382 mg, 1.1781 mmol) and B₂Pin₂ (359 mg, 1.4137 mmol) in 5 mL 1,4-dioxane were added KOAc (346.9 mg, 3.5343 mmol) and Pd(dppf)Cl₂DCM (144.31 mg, 0.1767 mmol), the mixture was stirred at 100° C. under N₂ for 16 h. After the reaction, the solvent was removed under reduced pressure, the residue was purified by column chromatography to afford 443 mg 1-(oxan-4-yl)-4-(4-(4,4,5,5-tetramethyl-1,3,2-dioxaborolan-2-yl)phenyl)piperidine as white solid. MS (ESI): mass calc'd. for C22H35BNO3 372.27, found 372 [M+1]⁺

### Step 3 Synthesis of tert-butyl (S)-(1-(3-methyl-5-(4-(1-(tetrahydro-2H-pyran-4-yl)piperidin-4-yl)phenyl)thiophene-2-carbonyl)pyrrolidin-3-yl)carbamate

To a solution of tert-butyl (S)-(1-(5-bromo-3-methylthiophene-2-carbonyl)pyrrolidin-3-yl)carbamate (200 mg, 0.5137 mmol) and 1-(oxan-4-yl)-4-(4-(4,4,5,5-tetramethyl-1,3,2-dioxaborolan-2-yl)phenyl)piperidine (228.9 mg, 0.6164 mmol) in dioxane/H₂O (9 mL, v/v=8:1) were added K₃PO₄ (327.1 mg, 1.5411 mmol) and Pd(dppf)Cl₂DCM (83.9 mg, 0.1027 mmol), the mixture was stirred at 95° C. under N₂ for 16 h. After the reaction, the solvent was removed under reduced pressure, the residue was purified by column chromatography to afford 150 mg tert-butyl (S)-(1-(3-methyl-5-(4-(1-(tetrahydro-2H-pyran-4-yl)piperidin-4-yl)phenyl)thiophene-2-carbonyl)pyrrolidin-3-yl)carbamate as white solid. MS (ESI): mass calc'd. for C31H44N3O4S 554.30, found 554 [M+1]⁺

### Step 4 Synthesis of (S)-(3-aminopyrrolidin-1-yl)(3-methyl-5-(4-(1-(tetrahydro-2H-pyran-4-yl)piperidin-4-yl)phenyl)thiophen-2-yl)methanone

To a solution of tert-butyl (S)-(1-(3-methyl-5-(4-(1-(tetrahydro-2H-pyran-4-yl)piperidin-4-yl)phenyl)thiophene-2-carbonyl)pyrrolidin-3-yl)carbamate (150 mg, 0.2709 mmol) in 2 mL EtOAc was added HCl (4 mL, 2M in EtOAc), the mixture was stirred at 25° C. for 12 h. After the reaction, the mixture was filtered and the filter cake was washed with EtOAc for 3 times to afford 51.6 mg of (S)-(3-aminopyrrolidin-1-yl)(3-methyl-5-(4-(1-(tetrahydro-2H-pyran-4-yl)piperidin-4-yl)phenyl)thiophen-2-yl)methanone (Compound 178) as yellow solid. MS (ESI): mass calc'd. for C26H36N3O2S 454.25, found 454 [M+1]⁺. ¹H NMR (400 MHz, MeOD) δ 7.62 (d, J=8.0 Hz, 2H), 7.35 (d, J=8.0 Hz, 2H), 7.25 (s, 1H), 4.10 (dd, J=11.6, 3.9 Hz, 2H), 4.01-3.98 (m, 2H), 3.87-3.70 (m, 5H), 3.48 (t, J=11.6 Hz, 3H), 3.20 (t, J=10.8 Hz, 2H), 2.99-2.93 (m, 1H), 2.50-2.43 (m, 1H), 2.36 (s, 3H), 2.14-2.10 (m, 7H), 1.90-1.82 (m, 2H).

### Example 40: Synthesis of (S)-(3-aminopyrrolidin-1-yl)(5-(4-(1-cyclohexylpiperidin-4-yl)phenyl)-3-methylthiophen-2-yl)methanone (Compound 179)

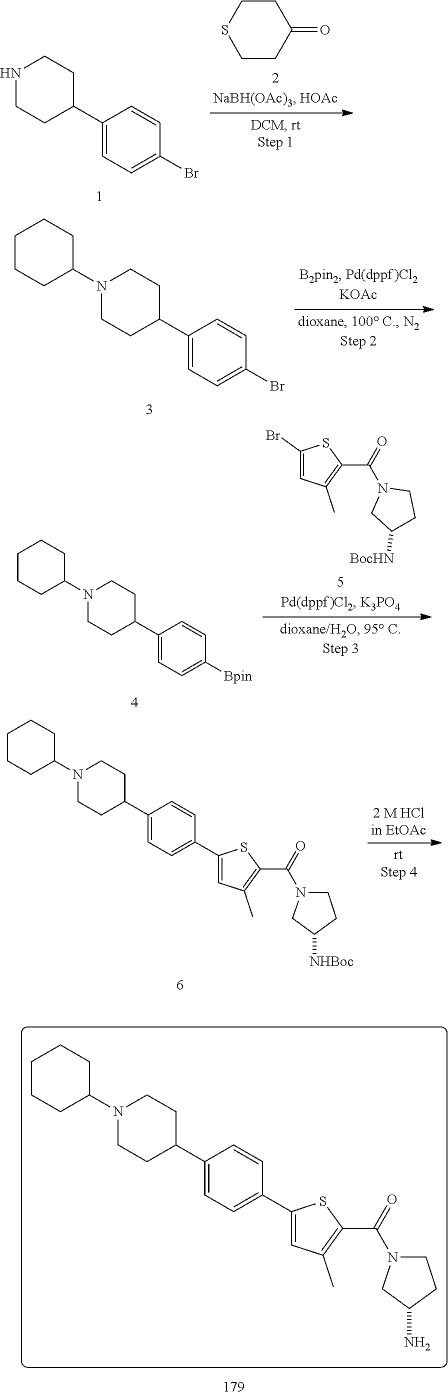

### Step 1 Synthesis of 4-(4-bromophenyl)-1-cyclopentylpiperidine

To a solution of 4-(4-bromophenyl)piperidine (240 mg, 1 mmol) in DCM (10 mL) was added cyclohexanone (98.1 mg, 1 mmol), NaBH(OAc)₃ (317.7 mg, 1.5 mmol) and HOAc (60 mg, 1 mmol). The mixture was stirred at rt for 3 h. The LCMS showed the reaction was completed and the desired product was found. The resulting mixture was washed with H₂O (20 mL) and extracted with DCM. The combined organic layer was washed with brine, dried over Na₂SO₄, filtered and concentrated under reduced pressure to give 4-(4-bromophenyl)-1-cyclohexylpiperidine (244 mg, 72% yield) as a yellow oil. LCMS (ESI) calc'd for C17H25BrN+ [M+H]+ m/z 322.12, found 322.

### Step 2 Synthesis of 1-cyclohexyl-4-(4-(4,4,5,5-tetramethyl-1,3,2-dioxaborolan-2-yl)phenyl)piperidine

To a solution of 4-(4-bromophenyl)-1-cyclohexylpiperidine (244 mg, 0.76 mmol) in dioxane (10 mL) were added B₂Pin₂ (192.3 mg, 0.76 mmol), KOAc (222.9 mg, 2.27 mmol) and Pd(dppf)Cl₂DCM (123.56 mg, 0.15 mmol). The mixture was stirred at 100° C. under N₂ for 16 h. After the reaction, the mixture was filtered and concentrated under reduced pressure. The residue was purified by flash chromatography EtOAc/MeOH=9:1 as eluent to give 1-cyclohexyl-4-(4-(4,4,5,5-tetramethyl-1,3,2-dioxaborolan-2-yl)phenyl)piperidine (158 mg, 53.7% yield) as a yellow oil. LCMS (ESI) calc'd for C23H37BNO2+ [M+H]+ m/z 370.29, found 370.

### Step 3 Synthesis of tert-butyl (S)-(1-(5-(4-(1-cyclohexylpiperidin-4-yl)phenyl)-3-methylthiophene-2-carbonyl)pyrrolidin-3-yl)carbamate

To a solution of tert-butyl (S)-(1-(5-bromo-3-methylthiophene-2-carbonyl)pyrrolidin-3-yl)carbamate (170 mg, 0.44 mmol) in dioxane/H₂O=8:1 (18 mL) were added 1-cyclohexyl-4-(4-(4,4,5,5-tetramethyl-1,3,2-dioxaborolan-2-yl)phenyl)piperidine (161.3 mg, 0.44 mmol), K₃PO₄ (278.1 mg, 1.31 mmol) and Pd(dppf)Cl₂DCM (71.3 mg, 0.09 mmol). The mixture was stirred at 95° C. under N₂ for 16 h. After the reaction, the mixture was filtered and concentrated under reduced pressure. The residue was purified by flash chromatography (MeOH/ethyl acetate=1/9) to give tert-butyl (S)-(1-(5-(4-(1-cyclohexylpiperidin-4-yl)phenyl)-3-methylthiophene-2-carbonyl)pyrrolidin-3-yl)carbamate (90 mg, 35.5% yield) as a yellow oil. LCMS (ESI) calc'd for C32H46N3O3S+ [M+H]+ m/z 552.33, found 552.

### Step 4 Synthesis of (S)-(3-aminopyrrolidin-1-yl)(5-(4-(1-cyclohexylpiperidin-4-yl)phenyl)-3-methylthiophen-2-yl)methanone

To a solution of tert-butyl (S)-(1-(5-(4-(1-cyclohexylpiperidin-4-yl)phenyl)-3-methylthiophene-2-carbonyl)pyrrolidin-3-yl)carbamate (90 mg, 0.16 mmol) in 4 mL EtOAc was added HCl (3 mL, 2M in EtOAc). The mixture was stirred at rt for 16 h. The LCMS showed the reaction was completed and the desired product was found. The mixture was concentrated under reduced pressure. The residue was purified by prep-HPLC to give (S)-(3-aminopyrrolidin-1-yl)(5-(4-(1-cyclohexylpiperidin-4-yl)phenyl)-3-methylthiophen-2-yl)methanone (Compound 179; 70 mg, 81.7% yield) as a yellow solid. LCMS (ESI) calc'd for C27H38N3OS+ [M+H]+ m/z 452.28, found 452. ¹H NMR (400 MHz, MeOD) δ 7.52 (d, J=7.9 Hz, 2H), 7.25 (t, J=7.4 Hz, 2H), 7.15 (s, 1H), 3.95-3.85 (m, 2H), 3.69 (ddt, J=34.8, 14.9, 7.0 Hz, 4H), 3.51 (d, J=11.3 Hz, 2H), 3.14 (s, 13H), 2.85 (s, 1H), 2.35 (dd, J=12.9, 6.1 Hz, 1H), 2.26 (s, 3H), 2.06 (dd, J=18.0, 8.9 Hz, 7H), 1.90-1.82 (m, 2H), 1.64 (d, J=12.7 Hz, 1H), 1.47 (dd, J=22.3, 11.4 Hz, 2H), 1.33 (dd, J=25.5, 12.7 Hz, 2H), 1.15 (dd, J=25.1, 12.3 Hz, 1H).

### Example 41: Synthesis of (S)-(3-aminopyrrolidin-1-yl)(5-(4-(1-cyclopentylpiperidin-4-yl)phenyl)-3-methylthiophen-2-yl)methanone (Compound 180)

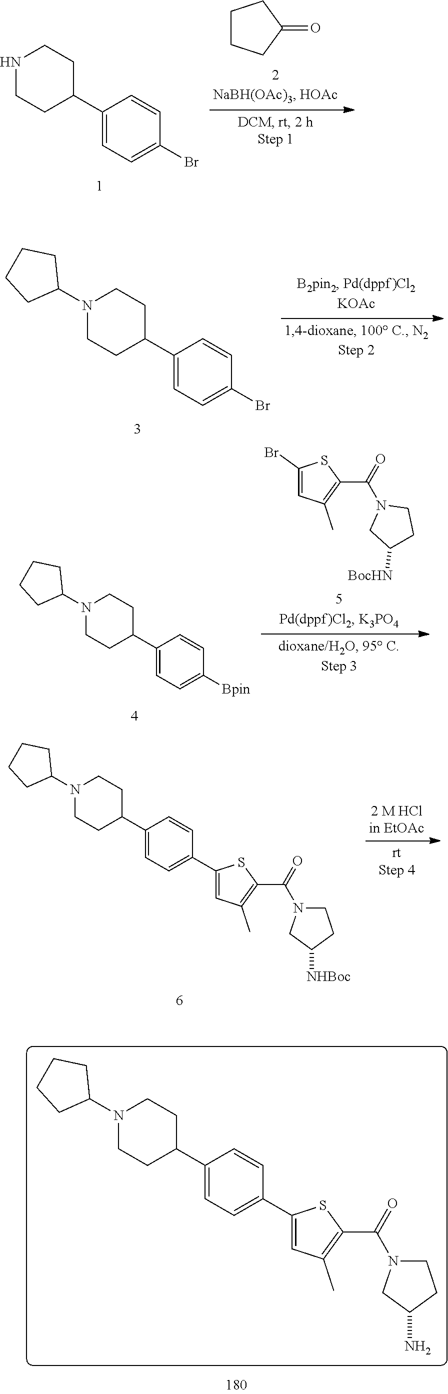

### Step 1 Synthesis of 4-(4-bromophenyl)-1-cyclopentylpiperidine

To a solution of 4-(4-bromophenyl)piperidine (240 mg, 1 mmol) in DCM (10 mL) was added cyclopentanone (84.7 mg, 1 mmol), NaBH(OAc)₃ (317.7 mg, 1.5 mmol) and HOAc (60 mg, 1 mmol). The mixture was stirred at rt for 3 h. The LCMS showed the reaction was completed and the desired product was found. The resulting mixture was washed with H₂O (20 mL) and extracted with DCM. The combined organic layer was washed with brine, dried over Na₂SO₄, filtered and concentrated under reduced pressure to give 4-(4-bromophenyl)-1-cyclopentylpiperidine (320 mg, 93.5% yield) as a yellow oil.

### Step 2 Synthesis of 1-cyclopentyl-4-(4-(4,4,5,5-tetramethyl-1,3,2-dioxaborolan-2-yl)phenyl)piperidine

To a solution of 4-(4-bromophenyl)-1-cyclopentylpiperidine (320 mg, 1.04 mmol) in dioxane (10 mL) were added B₂Pin₂ (263.6 mg, 1.04 mmol), KOAc (305.6 mg, 3.11 mmol) and Pd(dppf)Cl₂DCM (169.4 mg, 0.2 mmol). The mixture was stirred at 95° C. under N₂ for 16 h. After the reaction, the mixture was filtered and concentrated under reduced pressure. The residue was purified by flash chromatography EtOAc/MeOH=9:1 as eluent to give 1-cyclopentyl-4-(4-(4,4,5,5-tetramethyl-1,3,2-dioxaborolan-2-yl)phenyl)piperidine (232 mg, 59.8% yield) as a yellow oil.

### Step 3 Synthesis of tert-butyl (S)-(1-(5-(4-(1-cyclopentylpiperidin-4-yl)phenyl)-3-methylthiophene-2-carbonyl)pyrrolidin-3-yl)carbamate

To a solution of tert-butyl (S)-(1-(5-bromo-3-methylthiophene-2-carbonyl)pyrrolidin-3-yl)carbamate (250 mg, 0.64 mmol) in dioxane/H₂O=8:1 (18 mL) were added 1-cyclopentyl-4-(4-(4,4,5,5-tetramethyl-1,3,2-dioxaborolan-2-yl)phenyl)piperidine (228 mg, 0.64 mmol), K₃PO₄ (409 mg, 1.93 mmol) and Pd(dppf)Cl₂DCM (104.8 mg, 0.13 mmol). The mixture was stirred at 95° C. under N₂ for 16 h. After the reaction, the mixture was filtered and concentrated under reduced pressure. The residue was purified by flash chromatography (MeOH/ethyl acetate=1/9) to give tert-butyl (S)-(1-(5-(4-(1-cyclopentylpiperidin-4-yl)phenyl)-3-methylthiophene-2-carbonyl)pyrrolidin-3-yl)carbamate (100 mg, 27.5% yield) as a yellow oil.

### Step 4 Synthesis of (S)-(3-aminopyrrolidin-1-yl)(5-(4-(1-cyclopentylpiperidin-4-yl)phenyl)-3-methylthiophen-2-yl)methanone

To a solution of tert-butyl (S)-(1-(5-(4-(1-cyclopentylpiperidin-4-yl)phenyl)-3-methylthiophene-2-carbonyl)pyrrolidin-3-yl)carbamate (100 mg, 0.19 mmol) in 4 mL EtOAc was added HCl (3 mL, 2M in EtOAc). The mixture was stirred at rt for 16 h. The LCMS showed the reaction was completed and the desired product was found. The mixture was concentrated under reduced pressure. The residue was purified by prep-HPLC to give (S)-(3-aminopyrrolidin-1-yl)(5-(4-(1-cyclopentylpiperidin-4-yl)phenyl)-3-methylthiophen-2-yl)methanone (Compound 180; 70 mg, 81.7% yield) as a yellow solid. LCMS (ESI) calc'd for C26H36N3OS+ [M+H]+ m/z 438.26, found 438. ¹H NMR (400 MHz, MeOD) δ 7.64 (d, J=8.0 Hz, 2H), 7.36 (d, J=8.1 Hz, 2H), 7.27 (s, 1H), 4.06-3.98 (m, 2H), 3.91-3.79 (m, 2H), 3.74 (t, J=11.8 Hz, 3H), 3.64-3.53 (m, 1H), 3.17 (t, J=12.0 Hz, 2H), 2.97 (t, J=12.1 Hz, 1H), 2.54-2.42 (m, 1H), 2.38 (s, 1H), 2.28-2.10 (m, 3H), 2.10-2.00 (m, 6H), 1.85 (dd, J=24.6, 6.2 Hz, 4H), 1.73 (d, J=4.3 Hz, 2H).

### Example 42: Synthesis of (S)-(3-aminopyrrolidin-1-yl)(5-(4-(1-cyclobutylpiperidin-4-yl)phenyl)-3-methylthiophen-2-yl)methanone (Compound 181)

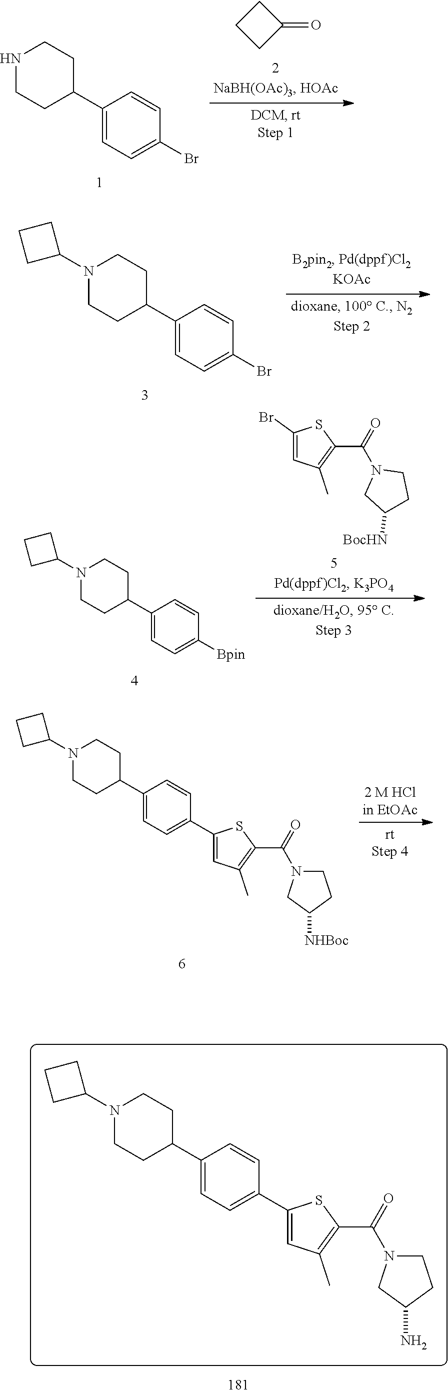

### Step 1 Synthesis of 4-(4-bromophenyl)-1-cyclobutylpiperidine

To a solution of 4-(4-bromophenyl)piperidine (240 mg, 1 mmol) in DCM (10 mL) was added cyclobutanone (70 mg, 1 mmol), NaBH(OAc)₃ (317.7 mg, 1.5 mmol) and HOAc (60 mg, 1 mmol). The mixture was stirred at rt for 3 h. The LCMS showed the reaction was completed and the desired MS was found. The resulting mixture was diluted with H₂O (20 mL) and extracted with EtOAc for 3 times. The combined organic layer was washed with brine, dried over Na₂SO₄, filtered and concentrated under reduced pressure to give 4-(4-bromophenyl)-1-cyclobutylpiperidine (290 mg, 93.7% yield) as a yellow solid. LCMS (ESI) calc'd for C15H21BrN+ [M+H]+ m/z 294.09, found 294.

### Step 2 Synthesis of 1-cyclobutyl-4-(4-(4,4,5,5-tetramethyl-1,3,2-dioxaborolan-2-yl)phenyl)piperidine

To a solution of 4-(4-bromophenyl)-1-cyclobutylpiperidine (290 mg, 0.98 mmol) in dioxane (10 mL) were added B₂Pin₂ (250 mg, 0.98 mmol), KOAc (290 mg, 3 mmol) and Pd(dppf)Cl₂DCM (160 mg, 0.19 mmol). The mixture was stirred at 100° C. under N₂ for 16 h. After the reaction, the mixture was filtered and concentrated under reduced pressure. The residue was purified by flash chromatography EtOAc/MeOH=9:1 as eluent to give 1-cyclobutyl-4-(4-(4,4,5,5-tetramethyl-1,3,2-dioxaborolan-2-yl)phenyl)piperidine (277 mg, 78.23% yield) as a yellow solid.

### Step 3 Synthesis of tert-butyl (S)-(1-(5-(4-(1-cyclobutylpiperidin-4-yl)phenyl)-3-methylthiophene-2-carbonyl)pyrrolidin-3-yl)carbamate

To a solution of tert-butyl (S)-(1-(5-bromo-3-methylthiophene-2-carbonyl)pyrrolidin-3-yl)carbamate (300 mg, 0.77 mmol) in dioxane/H₂O=8:1 (18 mL) were added 1-cyclobutyl-4-(4-(4,4,5,5-tetramethyl-1,3,2-dioxaborolan-2-yl)phenyl)piperidine (263 mg, 0.77 mmol), K₃PO₄ (490 mg, 2.3 mmol) and Pd(dppf)Cl₂DCM (125.8 mg, 0.15 mmol). The mixture was stirred at 95° C. under N₂ for 16 h. After the reaction, the mixture was filtered and concentrated under reduced pressure. The residue was purified by flash chromatography (petroleum ether/ethyl acetate=1/8) to give tert-butyl (S)-(1-(5-(4-(1-cyclobutylpiperidin-4-yl)phenyl)-3-methylthiophene-2-carbonyl)pyrrolidin-3-yl)carbamate (200 mg, 47% yield) as a yellow oil. LCMS (ESI) calc'd for C30H42N3O3S+ [M+H]+ m/z 524.30, found 524.

### Step 4 Synthesis of (S)-(3-aminopyrrolidin-1-yl)(5-(4-(1-cyclobutylpiperidin-4-yl)phenyl)-3-methylthiophen-2-yl)methanone

To a solution of tert-butyl (S)-(1-(5-(4-(1-cyclobutylpiperidin-4-yl)phenyl)-3-methylthiophene-2-carbonyl)pyrrolidin-3-yl)carbamate (200 mg, 0.38 mmol) in 4 mL EtOAc was added HCl (3 mL, 2M in EtOAc). The mixture was stirred at rt for 16 h. The LCMS showed the reaction was completed and the desired product was found. The mixture was concentrated under reduced pressure. The residue was purified by prep-HPLC to give (S)-(3-aminopyrrolidin-1-yl)(5-(4-(1-cyclobutylpiperidin-4-yl)phenyl)-3-methylthiophen-2-yl)methanone (Compound 181; 13.8 mg, 8% yield) as a white solid. LCMS (ESI) calc'd for C25H34N3OS+ [M+H]+ m/z 424.24, found 424. ¹H NMR (400 MHz, MeOD) δ 7.64 (d, J=8.3 Hz, 2H), 7.36 (t, J=8.8 Hz, 2H), 7.27 (s, 1H), 4.00 (t, J=7.3 Hz, 2H), 3.83 (ddt, J=11.7, 5.9, 5.0 Hz, 2H), 3.76-3.67 (m, 2H), 3.61 (d, J=12.5 Hz, 2H), 3.01-2.87 (m, 3H), 2.53-2.42 (m, 2H), 2.38 (d, J=4.7 Hz, 3H), 2.31 (dd, J=15.7, 6.0 Hz, 2H), 2.15 (t, J=13.4 Hz, 3H), 2.06-1.83 (m, 4H).

### Example 43: Synthesis of (S)-(3-aminopyrrolidin-1-yl)(3-methyl-5-(4-(1-propylpiperidin-4-yl)phenyl)thiophen-2-yl)methanone (Compound 182)

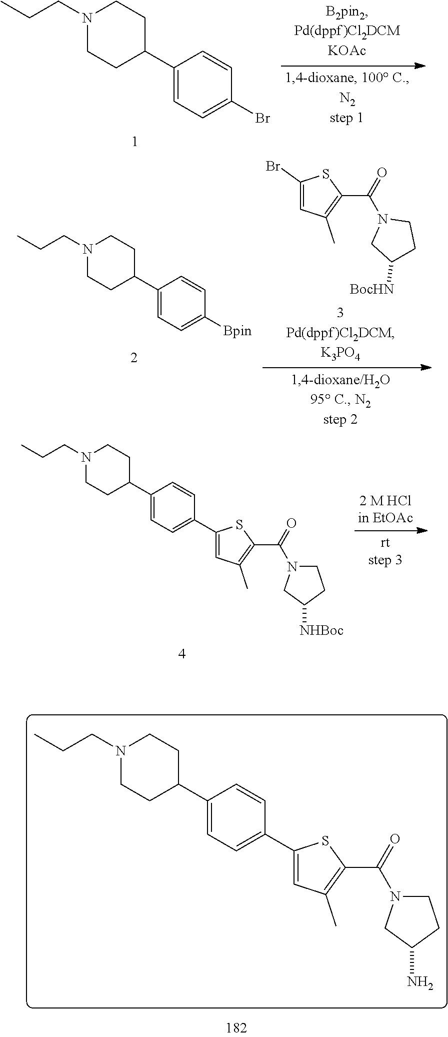

### Step 1 Synthesis of 1-propyl-4-(4-(4,4,5,5-tetramethyl-1,3,2-dioxaborolan-2-yl)phenyl)piperidine

To a solution of 4-(4-bromophenyl)-1-propylpiperidine (160 mg, 0.5669 mmol), B₂Pin₂ (172.8 mg, 0.6802 mmol) and KOAc (166.9 mg, 1.7 mmol) in 5 mL 1,4-dioxane was added Pd(dppf)Cl₂DCM, the mixture was stirred at 100° C. under N₂ for 16 h. After the reaction, the solvent was removed under reduced pressure, the residue was purified by column chromatography to afford 221 mg 1-propyl-4-(4-(4,4,5,5-tetramethyl-1,3,2-dioxaborolan-2-yl)phenyl)piperidine as white solid.

### Step 2 Synthesis of tert-butyl (S)-(1-(3-methyl-5-(4-(1-propylpiperidin-4-yl)phenyl)thiophene-2-carbonyl)pyrrolidin-3-yl)carbamate

To a solution of 1-propyl-4-(4-(4,4,5,5-tetramethyl-1,3,2-dioxaborolan-2-yl)phenyl)piperidine (172.6 mg, 0.524 mmol), tert-butyl N-((3S)-1-((5-bromo-3-methylthiophen-2-yl)carbonyl)pyrrolidin-3-yl)carbamate (170 mg, 0.4367 mmol) and K₃PO₄ (278.1 mg, 1.3101 mmol) in dioxane/H₂O (9 mL, v/v=8:1) was added Pd(dppf)Cl₂DCM (71.3 mg, 0.0873 mmol), the mixture was stirred at 95° C. under N₂ for 16 h. After the reaction, the solvent was removed under reduced pressure, the residue was purified by column chromatography to afford 132 mg tert-butyl N-((3S)-1-((3-methyl-5-(4-(1-propylpiperidin-4-yl)phenyl)thiophen-2-yl)carbonyl)pyrrolidin-3-yl)carbamate as yellowish solid.

### Step 3 Synthesis of (S)-(3-aminopyrrolidin-1-yl)(3-methyl-5-(4-(1-propylpiperidin-4-yl)phenyl)thiophen-2-yl)methanone

To a solution of tert-butyl (S)-(1-(3-methyl-5-(4-(1-propylpiperidin-4-yl)phenyl)thiophene-2-carbonyl)pyrrolidin-3-yl)carbamate (132 mg, 0.258 mmol) in 2 mL EtOAc was added HCl (4 mL, 2M in EtOAc), the mixture was stirred at 25° C. for 12 h. After the reaction, the reaction was filtered, the filter cake was washed with EtOAc for 3 times to afford 34.5 mg (S)-(3-aminopyrrolidin-1-yl)(3-methyl-5-(4-(1-propylpiperidin-4-yl)phenyl)thiophen-2-yl)methanone (Compound 182) as yellow solid. MS (ESI): mass calc'd. for C24H34N3OS 412.24, found 412 [M+H]⁺. ¹H NMR (400 MHz, MeOD) δ 7.62 (d, J=7.1 Hz, 2H), 7.34 (d, J=7.1 Hz, 2H), 7.25 (s, 1H), 4.00 (d, J=5.7 Hz, 2H), 3.81 (d, J=17.4 Hz, 2H), 3.70 (d, J=7.8 Hz, 3H), 3.13 (s, 4H), 3.00-2.92 (m, 1H), 2.47-2.43 (m, 1H), 2.36 (s, 3H), 2.16-2.01 (m, 5H), 1.84 (s, 1H), 1.05 (t, J=6.5 Hz, 3H).

### Example 44: Synthesis of (S)-(3-aminopyrrolidin-1-yl)(5-(4-(1-isopropylpiperidin-4-yl)phenyl)-3-methylthiophen-2-yl)methanone (Compound 183)

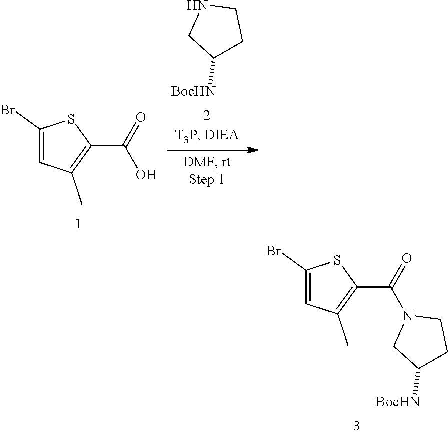

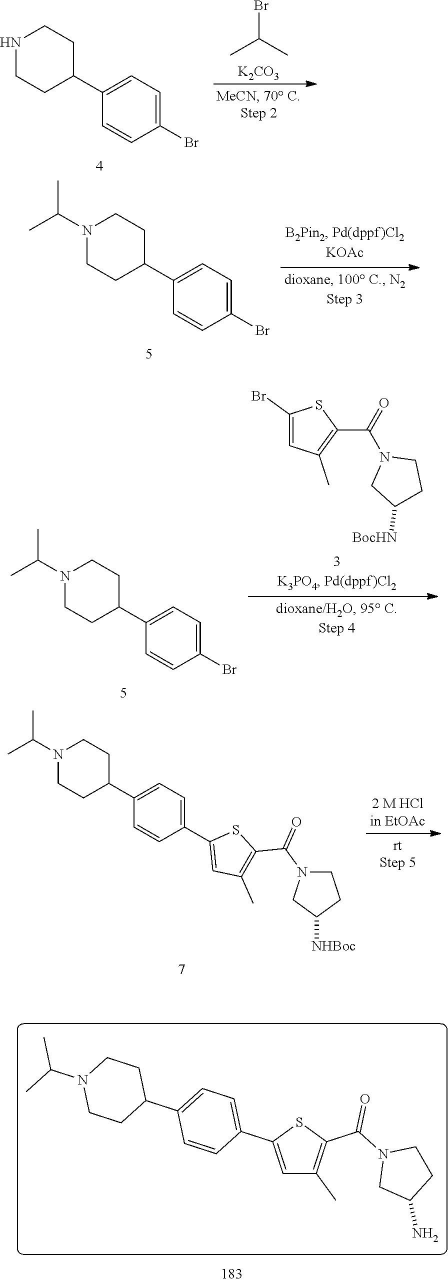

### Step 1 Synthesis of tert-butyl (S)-(1-(5-bromo-3-methylthiophene-2-carbonyl)pyrrolidin-3-yl)carbamate

To a solution of 5-bromo-3-methylthiophene-2-carboxylic acid (10.7 g, 48.4 mmol), tert-butyl (S)-pyrrolidin-3-ylcarbamate (9.02 g, 48.4 mmol) and DIEA (25.02 g, 193.6 mmol) in 150 mL DMF was added T3P (23.1 g, 72.6 mmol, 50 wt % in EtOAc), the mixture was stirred at 25° C. for 16 hours. After the reaction, the H₂O was added and the mixture was extracted with EtOAc for 3 times. Combined with EtOAc phases, washed with brine, dried over Na₂SO₄ and filtered, the solvent was removed under reduced pressure, the residue was purified by column chromatography to afford tert-butyl (S)-(1-(5-bromo-3-methylthiophene-2-carbonyl)pyrrolidin-3-yl)carbamate (11.4 g, 60.5% yield) as a pale yellow solid. LCMS (ESI) calc'd for C₁₅H₂₂BrN₂O₃S [M+H]+ m/z 391.05, found 391.0.

### Step 2 Synthesis of 4-(4-bromophenyl)-1-isopropylpiperidine

To a solution of 4-(4-bromophenyl)piperidine (10 g, 41.6 mmol) and 2-bromopropane (10.23 g, 83.2 mmol) in 100 mL MeCN was added K₂CO₃ (17.25 g, 124.8 mmol), the mixture was stirred at 70° C. for 16 hours. After the reaction, the solvent was removed under reduced pressure, the residue was purified by column chromatography using petroleum ether/ethyl acetate=7:3~1:1 as eluent to afford 4-(4-bromophenyl)-1-isopropylpiperidine (11.1 g, 93.51% yield) as a yellowish solid. ¹H NMR (400 MHz, CDCl3) δ 7.42-7.38 (m, 2H), 7.12-7.09 (m, 2H), 3.00 (d, J=11.6 Hz, 1H), 2.77-2.72 (m, 1H), 2.48-2.40 (m, 1H), 2.23 (td, J=11.6, 2.6 Hz, 1H), 1.84-1.68 (m, 4H), 1.08 (d, J=6.6 Hz, 6H).

### Step 3 Synthesis of 1-isopropyl-4-(4-(4,4,5,5-tetramethyl-1,3,2-dioxaborolan-2-yl)phenyl)piperidine

To a solution of 4-(4-bromophenyl)-1-isopropylpiperidine (10 g, 35.4 mmol), B₂Pin₂ (10.79 g, 42.48 mmol) and KOAc (10.42 g, 106.2 mmol) in 100 mL dioxane was added Pd(dppf)Cl₂DCM (2.89 g, 3.54 mmol), the mixture was stirred at 100° C. under N₂ for 16 hours. After the reaction, the solvent was removed under reduced pressure, the residue was purified by column chromatography using petroleum ether/ethyl acetate=1/1 as eluent to afford 1-isopropyl-4-(4-(4,4,5,5-tetramethyl-1,3,2-dioxaborolan-2-yl)phenyl)piperidine (12 g, 97.74% yield) as a yellow solid. LCMS (ESI) calc'd for C₂₀H₃₃BNO₂+ [M+H]+ m/z 330.25, found 330.2.

### Step 4 Synthesis of tert-butyl (S)-(1-(5-(4-(1-isopropylpiperidin-4-yl)phenyl)-3-methylthiophene-2-carbonyl)pyrrolidin-3-yl)carbamate

To a solution of tert-butyl (S)-(1-(5-bromo-3-methylthiophene-2-carbonyl)pyrrolidin-3-yl)carbamate (10 g, 25.7 mmol), 1-isopropyl-4-(4-(4,4,5,5-tetramethyl-1,3,2-dioxaborolan-2-yl)phenyl)piperidine (10.16 g, 30.84 mmol) and K₃PO₄ (16.37 g, 77.1 mmol) in 360 mL dioxane/H₂O=8:1 was added Pd(dppf)Cl₂DCM (2.1 g, 2.57 mmol), the mixture was stirred at 95° C. under N₂ for 16 hours. After the reaction, the solvent was removed under reduced pressure, the residue was purified by column chromatography using ethyl acetate/MeCN=3/7~1/0 as eluent to afford tert-butyl (S)-(1-(5-(4-(1-isopropylpiperidin-4-yl)phenyl)-3-methylthiophene-2-carbonyl)pyrrolidin-3-yl)carbamate (7.35 g, 54.47% yield) as a white solid. LCMS (ESI) calc'd for C₂₉H₄₂N₃O₃S+ [M+H]+ m/z 512.29, found 512.3.

### Step 5 Synthesis of (S)-(3-aminopyrrolidin-1-yl)(5-(4-(1-isopropylpiperidin-4-yl)phenyl)-3-methylthiophen-2-yl)methanone

To a solution of tert-butyl (S)-(1-(5-(4-(1-isopropylpiperidin-4-yl)phenyl)-3-methylthiophene-2-carbonyl)pyrrolidin-3-yl)carbamate (7.35 g, 14.4 mmol) in 100 mL EtOAc was added HCl (50 mL, 2M in EtOAc), the mixture was stirred at 25° C. for 16 hours. After the reaction, the mixture was filtered and the filtered cake was dissolved in pure water, which was dried through lyophilization to afford (S)-(3-aminopyrrolidin-1-yl)(5-(4-(1-isopropylpiperidin-4-yl)phenyl)-3-methylthiophen-2-yl)methanone (Compound 183; 5.69 g, 94% yield) as a yellow solid. LCMS (ESI) calc'd for C₂₄H₃₄N₃OS+ [M+H]+ m/z 412.24, found 412.3. ¹H NMR (400 MHz, MeOD) δ 7.61 (d, J=8.2 Hz, 2H), 7.34 (d, J=8.2 Hz, 2H), 7.24 (s, 1H), 4.02-3.98 (m, 2H), 3.89-3.82 (m, 1H), 3.80-3.69 (m, 2H), 3.58-3.55 (m, 3H), 3.25-3.18 (m, 2H), 2.99-2.91 (m, 1H), 2.50-2.41 (m, 1H), 2.35 (s, 3H), 2.19-2.12 (m, 5H), 1.42 (d, J=6.7 Hz, 6H).

### Example 45: Synthesis of (S)-(3-aminopyrrolidin-1-yl)(3-methyl-5-(1,2,3,4-tetrahydroisoquinolin-7-yl)thiophen-2-yl)methanone (Compound 198)

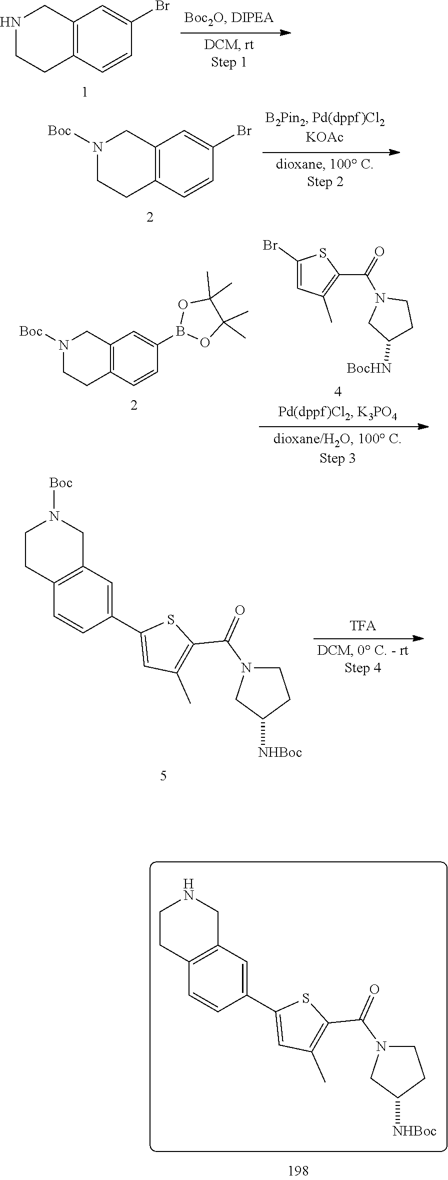 [SMILES: Brc1ccc2c(c1)CNCC2 ; CC(C)(C)OC(=O)N1CCc2ccc(Br)cc2C1 ; CC(C)(C)OC(=O)N1CCc2ccc(B3OC(C)(C)C(C)(C)O3)cc2C1 ; Cc1cc(Br)sc1C(=O)N1CC[C@H](NC(=O)OC(C)(C)C)C1 ; Cc1cc(-c2ccc3c(c2)CN(C(=O)OC(C)(C)C)CC3)sc1C(=O)N1CC[C@H](NC(=O)OC(C)(C)C)C1 ; Cc1cc(-c2ccc3c(c2)CNCC3)sc1C(=O)N1CC[C@H](NC(=O)OC(C)(C)C)C1]

### Step 1 Synthesis of tert-butyl 7-bromo-3,4-dihydroisoquinoline-2(1H)-carboxylate

To a solution of 7-bromo-1,2,3,4-tetrahydroisoquinoline (500 mg, 2.3 mmol) and Et₃N (1 mL, 7 mmol) in DCM (5 mL) was added Boc₂O (617.4 mg, 2.83 mmol), the mixture was stirred at room temperature for 16 h. After the reaction, the solvent was removed under reduced pressure, the residue was purified by column chromatography to afford tert-butyl 7-bromo-3,4-dihydroisoquinoline-2(1H)-carboxylate (700 mg, 95.1% yield) as a colorless oil.

### Step 2 Synthesis of tert-butyl 7-(4,4,5,5-tetramethyl-1,3,2-dioxaborolan-2-yl)-3,4-dihydroisoquinoline-2(1H)-carboxylate

To a solution of tert-butyl 7-bromo-3,4-dihydro-1H-isoquinoline-2-carboxylate (500 mg, 1.6 mmol), B₂Pin₂ (447 mg, 1.76 mmol) and KOAc (471 mg, 4.8 mmol) in 5 mL dioxane was added Pd(dppf)Cl₂ (234 mg, 0.32 mmol), the mixture was stirred at 100° C. for 16 h. After the reaction, the solvent was removed under reduced pressure, the residue was purified by flash column chromatography to afford tert-butyl 7-(4,4,5,5-tetramethyl-1,3,2-dioxaborolan-2-yl)-1,2,3,4-tetrahydroquinoline (560 mg, 97% yield) as a yellowish solid. LCMS (ESI) calc'd for C16H23BNO4+ [M-^{t}Bu+2H]+ m/z 304.17, found 304.

### Step 3 Synthesis of tert-butyl (S)-7-(5-(3-((tert-butoxycarbonyl)amino)pyrrolidine-1-carbonyl)-4-methylthiophen-2-yl)-3,4-dihydroisoquinoline-2(1H)-carboxylate

To a solution of tert-butyl 7-(4,4,5,5-tetramethyl-1,3,2-dioxaborolan-2-yl)-1,2,3,4-tetrahydroquinoline (203 mg, 0.56 mmol), tert-butyl (S)-(1-(5-bromo-3-methylthiophene-2-carbonyl)pyrrolidin-3-yl)carbamate (200 mg, 0.5137 mmol) and K₃PO₄ (327.1 mg, 1.5411 mmol) in 9 mL dioxane/H₂O (v:v=8:1) was added Pd(dppf)Cl₂ (150 mg, 0.20548 mmol), the mixture was stirred at 95° C. under N₂ for 16 hours. After the reaction, the solvent was removed under reduced pressure, the residue was purified by column chromatography to afford tert-butyl (S)-7-(5-(3-((tert-butoxycarbonyl)amino)pyrrolidine-1-carbonyl)-4-methylthiophen-2-yl)-3,4-dihydroisoquinoline-2(1H)-carboxylate (230 mg, 82.66% yield) as a brown solid. LCMS (ESI) calc'd for C29H40N3O5S+ [M+H]+ m/z 542.27, found 542.

### Step 4 Synthesis of (S)-(3-aminopyrrolidin-1-yl)(3-methyl-5-(1,2,3,4-tetrahydroisoquinolin-7-yl)thiophen-2-yl)methanone

To a solution of tert-butyl (S)-7-(5-(3-((tert-butoxycarbonyl)amino)pyrrolidine-1-carbonyl)-4-methylthiophen-2-yl)-3,4-dihydroisoquinoline-2(1H)-carboxylate (120 mg, 0.2215 mmol) in 5 mL DCM was added TFA (252.6 mg, 2.215 mmol) at 0° C., the mixture was slowly warmed up to room temperature and stirred at this temperature for 16 hours. After the reaction, the solvent and excess of TFA were removed under reduced pressure, the residue was purified by prep-HPLC to afford (S)-(3-aminopyrrolidin-1-yl)(3-methyl-5-(1,2,3,4-tetrahydroisoquinolin-7-yl)thiophen-2-yl)methanone (Compound 198; 36 mg, 47.58% yield) as a white solid. LCMS (ESI) calc'd for C19H24N3OS+ [M+H]+ m/z 342.16, found 342. ¹H NMR (400 MHz, MeOD) δ 7.48 (d, J=8.0 Hz, 1H), 7.42 (s, 1H), 7.21-7.18 (m, 2H), 4.31 (s, 2H), 3.90-3.86 (m, 2H), 3.77-3.58 (m, 3H), 3.43 (t, J=6.2 Hz, 2H), 3.04 (t, J=6.2 Hz, 2H), 2.39-2.30 (m, 1H), 2.25 (s, 3H), 2.09-2.02 (m, 1H).

### Example 46: Synthesis of (S)-(3-aminopyrrolidin-1-yl)(3-methyl-5-(1,2,3,4-tetrahydroisoquinolin-6-yl)thiophen-2-yl)methanone (Compound 199)

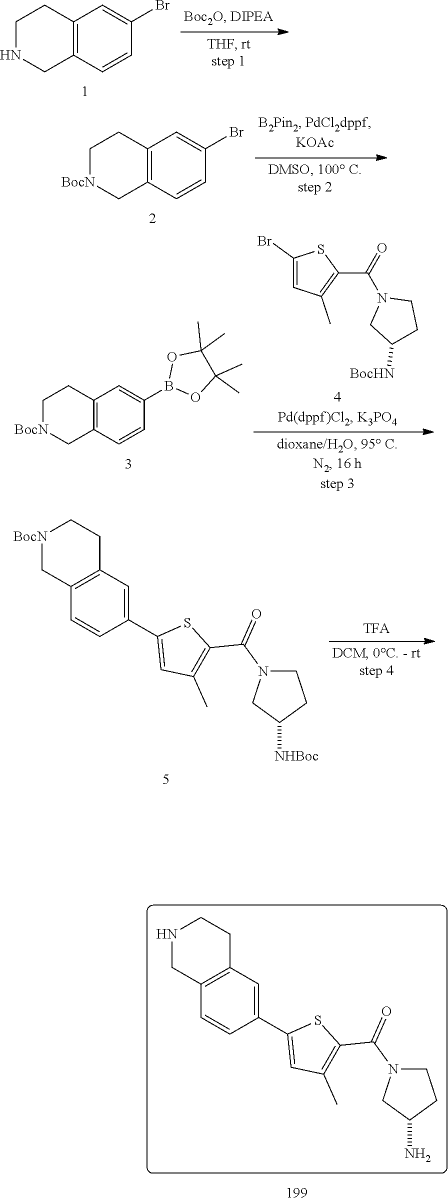 [SMILES: Brc1ccc2c(c1)CCNC2 ; CC(C)(C)OC(=O)N1CCc2cc(Br)ccc2C1 ; CC(C)(C)OC(=O)N1CCc2cc(B3OC(C)(C)C(C)(C)O3)ccc2C1 ; Cc1cc(Br)sc1C(=O)N1CC[C@H](NC(=O)OC(C)(C)C)C1 ; Cc1cc(-c2ccc3c(c2)CCN(C(=O)OC(C)(C)C)C3)sc1C(=O)N1CC[C@H](NC(=O)OC(C)(C)C)C1 ; Cc1cc(-c2ccc3c(c2)CCNC3)sc1C(=O)N1CC[C@H](N)C1]

### Step 1 Synthesis of tert-butyl 6-bromo-3,4-dihydroisoquinoline-2(1H)-carboxylate

To a solution of 6-bromo-1,2,3,4-tetrahydroisoquinoline (1 g, 4.715 mmol) and Boc₂O (2.05 g, 9.4 mmol) in dry THF (5 mL) was added DIPEA (1.82 g, 14.1 mmol) at 0° C.

The reaction mixture was allowed to warm to ambient temperature and stirred for 3 h. After the reaction, the solvent was removed under reduced pressure, the residue was purified by column chromatography to afford tert-butyl 6-bromo-3,4-dihydroisoquinoline-2(1H)-carboxylate (1.99 g) as an oil. MS (ESI): mass calc'd. for C10H9BrNO2 253.98, m/z found 254 [M-^{t}Bu+2H]⁺.

### Step 2 Synthesis of tert-butyl 6-(4,4,5,5-tetramethyl-1,3,2-dioxaborolan-2-yl)-3,4-dihydroisoquinoline-2(1H)-carboxylate

To a solution of tert-butyl 6-bromo-3,4-dihydroisoquinoline-2(1H)-carboxylate (1 g, 3.2 mmol), B₂Pin₂ (0.89 g, 3.5 mmol) and KOAc (0.94 g, 9.6 mmol) in 20 mL DMSO was added Pd(dppf)Cl₂ (0.07 g, 0.096 mmol), the mixture was stirred at 100° C. under N₂ for 16 hours. After the reaction, the mixture was diluted with H₂O and extracted with EtOAc for 3 times. Combined with EtOAc phase, washed with brine, dried over Na₂SO₄ and filtered, the solvent was removed under reduced pressure, the residue was purified by column chromatography to afford 0.54 g tert-butyl 6-(4,4,5,5-tetramethyl-1,3,2-dioxaborolan-2-yl)-3,4-dihydroisoquinoline-2(1H)-carboxylate as light green solid.

### Step 3 Synthesis of tert-butyl (S)-6-(5-(3-((tert-butoxycarbonyl)amino)pyrrolidine-1-carbonyl)-4-methylthiophen-2-yl)-3,4-dihydroisoquinoline-2(1H)-carboxylate

To a solution of tert-butyl (S)-(1-(5-bromo-3-methylthiophene-2-carbonyl)pyrrolidin-3-yl)carbamate (200 mg, 0.5137 mmol), tert-butyl 6-(4,4,5,5-tetramethyl-1,3,2-dioxaborolan-2-yl)-3,4-dihydroisoquinoline-2(1H)-carboxylate (222.1 mg, 0.6164 mmol) and K₃PO₄ (327.1 mg, 1.5411 mmol) in dioxane/H₂O (9 mL, v:v=8:1) was added Pd(dppf)Cl₂DCM (83.9 mg, 0.1027 mmol), the mixture was stirred at 95° C. under N₂ for 16 hours. After the reaction, the solvent was removed under reduced pressure, the residue was purified by column chromatography to afford tert-butyl (S)-6-(5-(3-((tert-butoxycarbonyl)amino)pyrrolidine-1-carbonyl)-4-methylthiophen-2-yl)-3,4-dihydroisoquinoline-2(1H)-carboxylate (267 mg) as a yellow oil.

### Step 4 Synthesis of (S)-(3-aminopyrrolidin-1-yl)(3-methyl-5-(1,2,3,4-tetrahydroisoquinolin-6-yl)thiophen-2-yl)methanone

To a solution of tert-butyl (S)-6-(5-(3-((tert-butoxycarbonyl)amino)pyrrolidine-1-carbonyl)-4-methylthiophen-2-yl)-3,4-dihydroisoquinoline-2(1H)-carboxylate (267 mg, 0.4929 mmol) in DCM (6 mL) was slowly added TFA (0.5 mL) at 0° C., the mixture was stirred at 25° C. for 16 hours. After the reaction, the DCM and TFA were removed under reduced pressure, the residue was purified by prep-HPLC to afford (S)-(3-aminopyrrolidin-1-yl)(3-methyl-5-(1,2,3,4-tetrahydroisoquinolin-6-yl)thiophen-2-yl)methanone (Compound 199; 60 mg) as a yellow solid. MS (ESI): mass calc'd. for C19H24N3OS 342.16, found 342 [M+H]⁺. ¹H NMR (400 MHz, MeOD) δ 7.55-7.53 (m, 2H), 7.28-7.25 (m, 2H), 4.37 (s, 2H), 3.98-3.95 (m, 2H), 3.87-3.65 (m, 3H), 3.52 (t, J=6.3 Hz, 2H), 3.15 (t, J=6.1 Hz, 2H), 2.47-2.38 (m, 1H), 2.35 (s, 3H), 2.17-2.09 (m, 1H).

### Example 47: Synthesis of ((S)-3-aminopyrrolidin-1-yl)(5-(4-(1-(dimethylamino)ethyl)phenyl)-3-methylthiophen-2-yl)methanone (Compound 200)

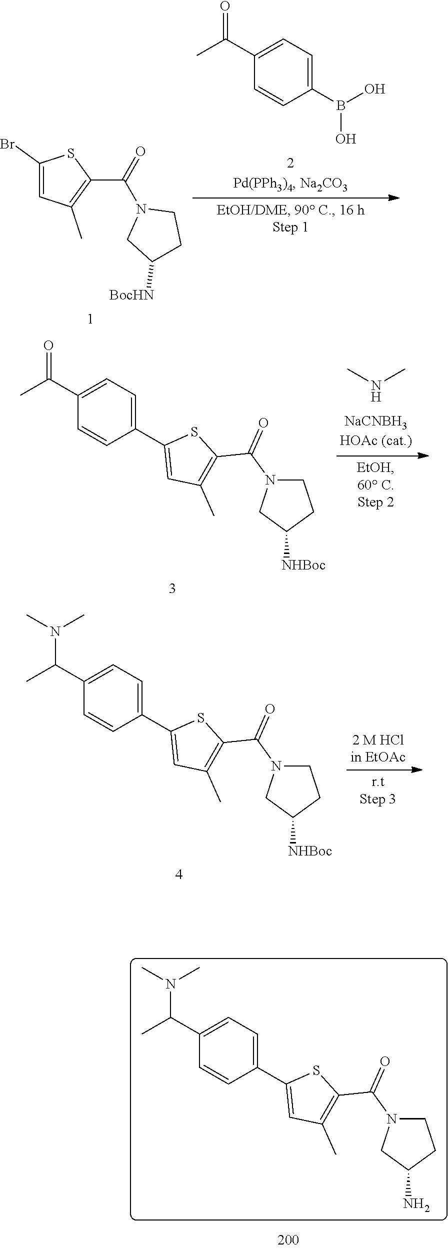

### Step 1 tert-butyl (S)-(1-(5-(4-acetylphenyl)-3-methylthiophene-2-carbonyl)pyrrolidin-3-yl)carbamate

To a solution of tert-butyl (S)-(1-(5-bromo-3-methylthiophene-2-carbonyl)pyrrolidin-3-yl)carbamate (389 mg, 1 mmol) in EtOH:DME=1:1 (16 mL) were added (4-acetylphenyl)boronic acid (180 mg, 1.1 mmol), Na₂CO₃ (317.7 mg, 3 mmol) and Pd(PPh₃)₄ (231 mg, 0.2 mmol). The mixture was stirred at 90° C. under N₂ for 16 h. After cooled to rt, the resulting mixture was purified by flash chromatography (petroleum ether/ethyl acetate=1/1) to give tert-butyl (S)-(1-(5-(4-acetylphenyl)-3-methylthiophene-2-carbonyl)pyrrolidin-3-yl)carbamate (450 mg, 99.8% yield) as a yellow solid. LCMS (ESI) calc'd for C23H28N2O4S+ [M+H]+ m/z 428.18, found 428.2.

### Step 2 Synthesis of tert-butyl ((3S)-1-(5-(4-(1-(dimethylamino)ethyl)phenyl)-3-methylthiophene-2-carbonyl)pyrrolidin-3-yl)carbamate

To a solution of tert-butyl (S)-(1-(5-(4-acetylphenyl)-3-methylthiophene-2-carbonyl)pyrrolidin-3-yl)carbamate (300 mg, 0.7 mmol) in EtOH (10 mL) was add dimethylamine (315.6 mg, 7 mmol). The mixture was stirred at rt for 2 h, then NaBH₃CN (132 mg, 2.1 mmol) was added. The reaction was stirred at 60° C. for 16 h. LCMS showed product was formed. After cooled to rt, the resulted mixture was diluted with H₂O (50 ml) and extracted with ethyl acetate (2×50 mL). The combined organic layers were washed with brine (50 mL), dried over Na₂SO₄, filtered and concentrated under reduced pressure. The residue was purified by flash chromatography (petroleum ether/ethyl acetate=8/1) to give tert-butyl ((3S)-1-(5-(4-(1-(dimethylamino)ethyl)phenyl)-3-methylthiophene-2-carbonyl)pyrrolidin-3-yl)carbamate (250 mg, 70% yield) as a yellow oil. LCMS (ESI) calc'd for C25H36N3O3S+ [M+H]+ m/z 458.25, found 458.2.

### Step 3 Synthesis of ((S)-3-aminopyrrolidin-1-yl)(5-(4-(1-(dimethylamino)ethyl)phenyl)-3-methylthiophen-2-yl)methanone

To a solution of tert-butyl ((3S)-1-(5-(4-(1-(dimethylamino)ethyl)phenyl)-3-methylthiophene-2-carbonyl)pyrrolidin-3-yl)carbamate (200 mg, 0.44 mmol) in DCM (5 mL) was added TFA (1 mL). The mixture was stirred at rt for 16 h. The LCMS showed the reaction was completed and the desired MS was found. The reaction was concentrated under reduced pressure and the residue was purified by prep-HPLC to give the ((S)-3-aminopyrrolidin-1-yl)(5-(4-(1-(dimethylamino)ethyl)phenyl)-3-methylthiophen-2-yl)methanone (Compound 200; 14.1 mg, 8.5% Yield) as a white solid. LCMS (ESI) calc'd for C20H28N3OS+ [M+H]+ m/z 358.20, found 358.2. ¹H NMR (400 MHz, MeOD) δ 7.67 (d, J=8.0 Hz, 2H), 7.47 (d, J=8.0 Hz, 2H), 7.25 (s, 1H), 4.40 (q, J=6.7 Hz, 1H), 3.96-3.85 (m, 2H), 3.76 (dt, J=14.7, 7.5 Hz, 1H), 3.70-3.57 (m, 2H), 2.70 (s, 6H), 2.35 (dd, J=13.1, 6.3 Hz, 1H), 2.26 (s, 3H), 2.07 (d, J=5.5 Hz, 1H), 1.65 (d, J=6.9 Hz, 3H).

The following compounds are prepared analogously to the methods described in this example:

| Compound | Synthesis |
|---|---|
| 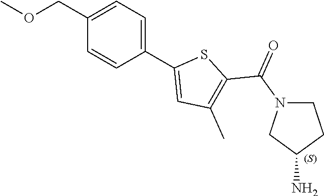 | For step 2, a corresponding aryl boronic acid can be used to synthesize compound 211 |
| 211 | |
| 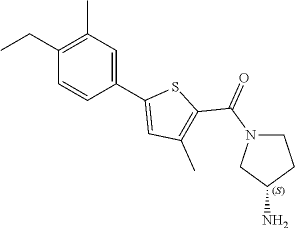 | For step 2, a corresponding aryl boronic acid can be used to synthesize compound 220 |
| 220 | |
| 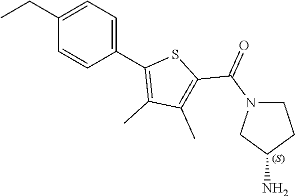 | For step 2, a corresponding aryl boronic acid and bromo thiophene can be used to synthesize compound 233 |
| 233 | |
| 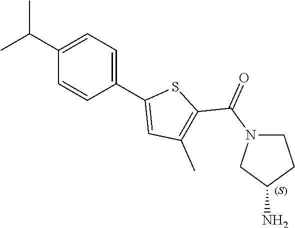 | For step 2, a corresponding aryl boronic acid can be used to synthesize compound 230 |
| 230 | |
| 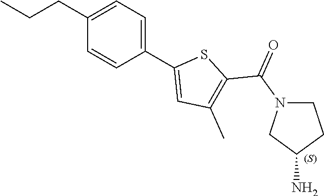 | For step 2, a corresponding aryl boronic acid can be used to synthesize compound 227 |
| 227 | |
| 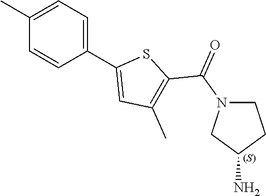 | For step 2, a corresponding aryl boronic acid can be used to synthesize compound 225 |
| 225 | |
| 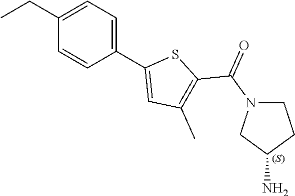 | For step 2, a corresponding aryl boronic acid can be used to synthesize compound 228 |
| 228 | |

### Example 48: Synthesis of (S)-(3-aminopyrrolidin-1-yl)(3-methyl-5-(4-(1-methylpiperidin-4-yl)phenyl)thiophen-2-yl)methanone (Compound 201)

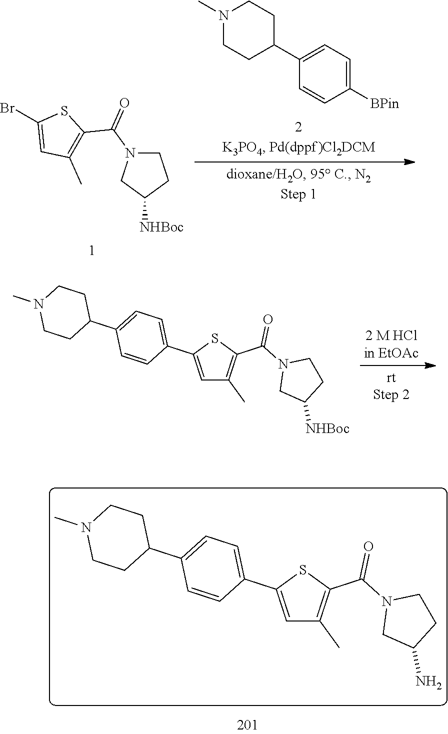

### Step 1 Synthesis of tert-butyl (S)-(1-(3-methyl-5-(4-(1-methylpiperidin-4-yl)phenyl)thiophene-2-carbonyl)pyrrolidin-3-yl)carbamate

To a solution of tert-butyl (S)-(1-(5-bromo-3-methylthiophene-2-carbonyl)pyrrolidin-3-yl)carbamate (420 mg, 1.08 mmol) in dioxane/H₂O=8:1 (18 mL) were added 1-methyl-4-(4-(4,4,5,5-tetramethyl-1,3,2-dioxaborolan-2-yl)phenyl)piperidine (325 mg, 1.08 mmol), K₃PO₄ (687 mg, 3.24 mmol) and Pd(dppf)Cl₂DCM (176.1 mg, 0.22 mmol). The mixture was stirred at 95° C. for 16 h. After the reaction, the mixture was filtered and concentrated under reduced pressure. The residue was purified by flash chromatography (MeOH/ethyl acetate=1/9) to give tert-butyl (S)-(1-(3-methyl-5-(4-(1-methylpiperidin-4-yl)phenyl)thiophene-2-carbonyl)pyrrolidin-3-yl)carbamate (200 mg, 36.4% yield) as a yellow solid.

### Step 2 Synthesis of (S)-(3-aminopyrrolidin-1-yl)(3-methyl-5-(4-(1-methylpiperidin-4-yl)phenyl)thiophen-2-yl)methanone

To a solution of tert-butyl (S)-(1-(3-methyl-5-(4-(1-methylpiperidin-4-yl)phenyl)thiophene-2-carbonyl)pyrrolidin-3-yl)carbamate (200 mg, 0.41 mmol) in 5 mL EtOAc was added HCl (3 mL, 2M in EtOAc). The mixture was stirred at rt for 16 h. The LCMS showed the reaction was completed and the desired product was found. The mixture was concentrated under reduced pressure. The residue was purified by prep-HPLC to give the (S)-(3-aminopyrrolidin-1-yl)(3-methyl-5-(4-(1-methylpiperidin-4-yl)phenyl)thiophen-2-yl)methanone (Compound 201; 31.8 mg, 19.8% Yield) as a white solid. LCMS (ESI) calc'd for C22H30N3OS+ [M+H]+ m/z 384, found 384. ¹H NMR (400 MHz, MeOD) δ 7.58 (d, J=8.3 Hz, 2H), 7.30 (d, J=8.3 Hz, 2H), 7.21 (s, 1H), 3.79 (dt, J=11.9, 7.1 Hz, 2H), 3.69-3.56 (m, 2H), 3.36 (d, J=5.1 Hz, 1H), 3.04 (d, J=11.8 Hz, 2H), 2.66-2.52 (m, 1H), 2.36 (s, 3H), 2.34 (s, 3H), 2.23 (dd, J=11.8, 2.9 Hz, 2H), 2.18 (d, J=2.7 Hz, 1H), 1.88 (d, J=13.6 Hz, 2H), 1.83 (d, J=3.7 Hz, 2H), 1.81-1.76 (m, 1H).

### Example 49: Synthesis of (S)-(3-aminopyrrolidin-1-yl)(5-(4-((dimethylamino)methyl)phenyl)-3-methylthiophen-2-yl)methanone (Compound 217)

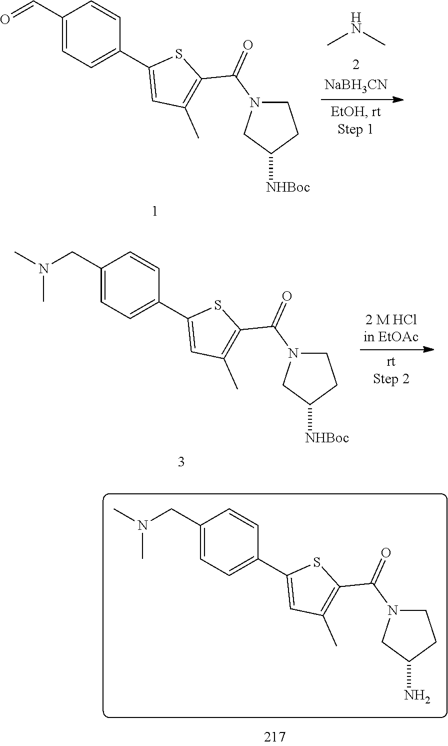

### Step 1 tert-butyl (S)-(1-(5-(4-((dimethylamino)methyl)phenyl)-3-methylthiophene-2-carbonyl)pyrrolidin-3-yl)carbamate

To a solution of tert-butyl (S)-(1-(5-(4-formylphenyl)-3-methylthiophene-2-carbonyl)pyrrolidin-3-yl)carbamate (200 mg, 0.48 mmol) in EtOH (10 mL) were added dimethylamine (1.5 mL, 2M in MeOH), NaBH₃CN (45.6 mg, 0.73 mmol), then 8 drops of HOAc was added. The mixture was stirred at rt for 16 h. The LCMS showed the reaction was completed and the desired MS was found. The resulting mixture was concentrated under reduced pressure and the residue was purified by flash chromatography (MeOH/ethyl acetate=1/10) to give tert-butyl (S)-(1-(5-(4-((dimethylamino)methyl)phenyl)-3-methylthiophene-2-carbonyl)pyrrolidin-3-yl)carbamate (140 mg, 62% yield) as a yellow oil. LCMS (ESI) calc'd for C24H34N3O3S+ [M+H]+ m/z 444.22, found 444.

### Step 2 Synthesis of (S)-(3-aminopyrrolidin-1-yl)(5-(4-((dimethylamino)methyl)phenyl)-3-methylthiophen-2-yl)methanone

To a solution of tert-butyl (S)-(1-(5-(4-((dimethylamino)methyl)phenyl)-3-methylthiophene-2-carbonyl)pyrrolidin-3-yl)carbamate (200 mg, 0.45 mmol) in 4 mL EtOAc was added HCl (3 mL, 2M in EtOAc). The mixture was stirred at rt for 16 h. The LCMS showed the reaction was completed and the desired product was found. The mixture was concentrated under reduced pressure. The residue was purified by prep-HPLC to give (S)-(3-aminopyrrolidin-1-yl)(5-(4-((dimethylamino)methyl)phenyl)-3-methylthiophen-2-yl)methanone (Compound 217, 13 mg, 8% yield) as a yellow solid. LCMS (ESI) calc'd for C19H26N3OS+ [M+H]+ m/z 344.17, found 344. ¹H NMR (400 MHz, MeOD) δ 7.80 (d, J=8.1 Hz, 2H), 7.62 (d, J=8.1 Hz, 2H), 7.37 (d, J=7.5 Hz, 1H), 4.38 (s, 2H), 4.09-3.96 (m, 2H), 3.93-3.83 (m, 1H), 3.83-3.68 (m, 2H), 2.90 (s, 6H), 2.55-2.43 (m, 1H), 2.39 (s, 3H), 2.19 (dd, J=14.5, 9.2 Hz, 1H).

The following compounds are prepared analogously to the methods described in this example:

| Compound | Synthesis |
|---|---|
| 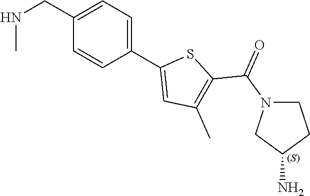 | For step 1, a corresponding amine can be used to synthesize compound 218 |
| 218 | |
| 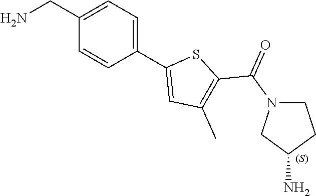 | For step 1, a corresponding amine can be used to synthesize compound 219; a protecting step may be used |
| 219 | |

### Example 50: Synthesis of (S)-(3-aminopyrrolidin-1-yl)(3-methyl-5-(4-(pyrrolidin-1-ylmethyl)phenyl)thiophen-2-yl)methanone (Compound 209)

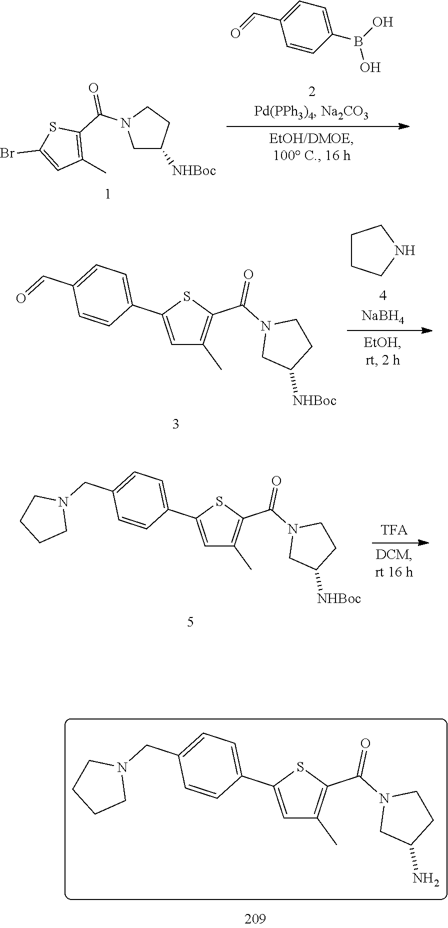 [SMILES: O=Cc1ccc(B(O)O)cc1 ; Cc1cc(Br)sc1C(=O)N1CC[C@H](NC(=O)OC(C)(C)C)C1 ; Cc1cc(-c2ccc(C=O)cc2)sc1C(=O)N1CC[C@H](NC(=O)OC(C)(C)C)C1 ; C1CCNC1 ; Cc1cc(-c2ccc(CN3CCCC3)cc2)sc1C(=O)N1CC[C@H](NC(=O)OC(C)(C)C)C1 ; Cc1cc(-c2ccc(CN3CCCC3)cc2)sc1C(=O)N1CC[C@H](N)C1]

Step 1 Synthesis of tert-butyl (S)-{1-[5-(4-formylphenyl)-3-methylthiophene-2-carbonyl]pyrrolidin-3-yl}carbamate

To a microwave vial was added tert-Butyl (S)-(1-(5-bromo-3-methylthiophene-2-carbonyl)pyrrolidin-3-yl)carbamate (0.520 mmol), Pd(PPh₃)₄ (60.0 mg, 0.052 mmol), Na₂CO₃ (1.56 mmol) and [4-(pyridin-4-yl)phenyl]boranediol (0.620 mmol), and the reaction vessel was purged with N₂ for three times. Then degassed EtOH and 1, 2-dimethoxyethane (5.0 mL, v:v=1:1) were added to the mixture, and the reaction vessel was sealed, heated to 100° C. and stirred for 16 hours. LCMS showed that the starting material was consumed and the desired mass was observed. The solution was cooled to room temperature, dilute with EtOAc, and filtered through a short pad of Celite. The filtrate was concentrated to afford the crude product, which was used for the next step without further purification. MS (ESI): mass calc'd. for C22H26N2O4S+ [M+H]+ m/z 415.16, found 415.2.

### Step 2 Synthesis of tert-butyl (S)-{1-[3-methyl-5-(4-pyrrolidin-1-ylmethylphenyl)thiophene-2-carbonyl]pyrrolidin-3-yl}carbamate

A mixture of tert-butyl N-[(3S)-1-{[5-(4-formylphenyl)-3-methylthiophen-2-yl]carbonyl}pyrrolidin-3-yl]carbamate (200 mg, 0.48 mmol) and pyrrolidine (137 mg, 1.93 mmol) in 2 mL of EtOH was degassed and purged with N₂ for 3 times, and then the mixture was stirred at 25° C. for 2 h under N₂ atmosphere. LCMS showed that the starting material was consumed and the desired mass was observed. Then NaBH₃CN (60.6 mg, 0.96 mmol) was added in portion wise to the solution, and the reaction was stirred at 25° C. for 2 h. The reaction mixture was partitioned between 10 mL of NH₄C₁ (aq.) and 10 mL of ethyl acetate. The organic phase was separated, washed with 4 mL of brine, dried over Na₂SO₄, filtered and concentrated under reduced pressure to give the crude product (20 mg, 9% yield) as a yellow solid. MS (ESI): mass calc'd. for C26H35N3O3S+ [M+H]+ m/z 470.24, found 470.2.

### Step 3 Synthesis of (S)-(3-aminopyrrolidin-1-yl)(3-methyl-5-(4-(pyrrolidin-1-ylmethyl)phenyl)thiophen-2-yl)methanone

A mixture of tert-butyl N-{(3S)-1-[3-methyl-5-(4-pyrrolidin-1-ylmethylphenyl)thiophen-2-yl]carbonylpyrrolidin-3-yl}carbamate (200 mg, 0.43 mmol) and TFA (854.7 mg, 7.5 mmol) in dichloromethane (10 mL) was degassed and purged with N₂ for 3 times, then the mixture was stirred at 25° C. for 16 hours under N₂ atmosphere. LCMS showed that the starting material was consumed and the desired mass was observed. The solution was concentrated under reduced pressure to give the crude product. The residue was purified by prep-HPLC to give the desired product (Compound 209, 52.7 mg, 33.5% yield) as white solid. MS (ESI): mass calc'd. for C21H27N3OS+ [M+H]+ m/z 371.1, found 371.1.

¹H NMR (400 MHz, DMSO) δ 7.68 (d, J=8.0 Hz, 2H), 7.49 (d, J=8.0 Hz, 2H), 7.41 (s, 1H), 4.05 (s, 2H), 3.88 (s, 1H), 3.74 (m, 2H), 3.65-3.48 (m, 2H), 2.91 (s, 4H), 2.29 (s, 3H), 2.24 (m, 1H), 2.07-1.94 (m, 1H), 1.85 (s, 6H).

The following compounds are prepared analogously to the methods described in this example:

| Compound | Synthesis |
|---|---|
| 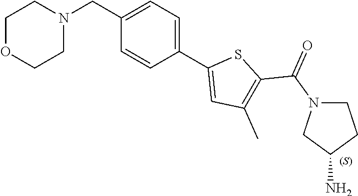 | For step 2, morpholine is used instead of pyrrolidine |
| 131 | |
| 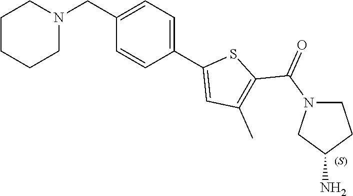 | For step 2, piperidine is used instead of pyrrolidine |
| 163 | |

### Example 51: Synthesis of (S)-1-{4-[5-(3-aminopyrrolidine-1-carbonyl)-4-methylthiophen-2-yl]benzyl}piperidin-2-one (Compound 208)

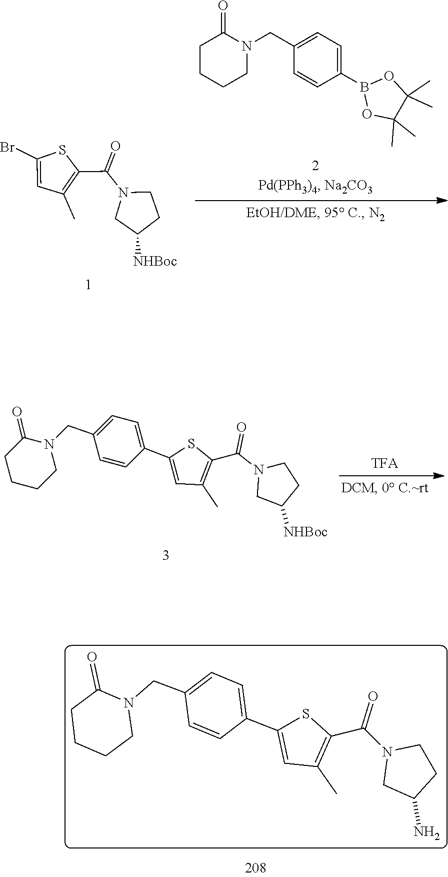 [SMILES: Cc1cc(Br)sc1C(=O)N1CC[C@H](NC(=O)OC(C)(C)C)C1 ; CC1(C)OB(c2ccc(CN3CCCCC3=O)cc2)OC1(C)C ; Cc1cc(-c2ccc(CN3CCCCC3=O)cc2)sc1C(=O)N1CC[C@H](NC(=O)OC(C)(C)C)C1 ; Cc1cc(-c2ccc(CN3CCCCC3=O)cc2)sc1C(=O)N1CC[C@H](N)C1]

### Step 1 Synthesis of tert-butyl (S)-(1-(3-methyl-5-(4-((2-oxopiperidin-1-yl)methyl)phenyl)thiophene-2-carbonyl)pyrrolidin-3-yl)carbamate

To a solution of tert-butyl (S)-[1-(5-bromo-3-methylthiophene-2-carbonyl)pyrrolidin-3-yl]carbamate (200 mg, 0.52 mmol), 1-[4-(4,4,5,5-tetramethyl-1,3,2-dioxaborolan-2-yl)benzyl]piperidin-2-one (194.3 mg, 0.616 mmol) and sodium carbonate (163.4 mg, 1.54 mmol) in EtOH/DME (v:v=1:1, 10 mL) was added Pd(PPh₃)₄ (59.4 mg, 0.051 mmol), then the reaction mixture was stirred at 95° C. for 16 h. After the reaction was completed, the mixture was cooled to room temperature, diluted with water, and extracted with EtOAc for 3 times. The combined organic phase was washed with brine, dried over Na₂SO₄, filtered and concentrated under reduced pressure. The residue was purified by column chromatography to afford tert-butyl (S)-1-{3-methyl-5-[4-(2-oxopiperidin-1-ylmethyl)phenyl]thiophene-2-carbonyl}pyrrolidin-3-ylcarbamate (180 mg, 69.71% yield) as yellowish solid. LCMS (ESI): calc'd. for C27H36N3O4S+ [M+H]+ m/z 498.24, found 498.3.

### Step 2 Synthesis of (S)-1-{4-[5-(3-aminopyrrolidine-1-carbonyl)-4-methylthiophen-2-yl]benzyl}piperidin-2-one

To a solution of tert-butyl (S)-(1-(3-methyl-5-(4-((2-oxopiperidin-1-yl)methyl)phenyl)thiophene-2-carbonyl)pyrrolidin-3-yl)carbamate (180 mg, 0.362 mmol) in DCM (10 mL) was added TFA (725.9 mg, 6.37 mmol) dropwise at 0° C., then the reaction mixture was stirred at room temperature for 16 h. After the reaction was completed, the solution was concentrated under reduced pressure, and the residue was purified by prep-HPLC to afford (S)-1-{4-[5-(3-aminopyrrolidine-1-carbonyl)-4-methylthiophen-2-yl]benzyl}piperidin-2-one (Compound 208, 28.6 mg, 19.49% yield) as white solid. LCMS (ESI) calc'd for C22H28N3O2S+ [M+H]+ m/z 398.19, found 398.1. ¹H NMR (400 MHz, DMSO) δ 7.61 (d, J=8.2 Hz, 2H), 7.34 (s, 1H), 7.28 (d, J=8.2 Hz, 2H), 4.51 (s, 2H), 3.75-3.65 (m, 3H), 3.57-3.41 (m, 3H), 3.19 (s, 2H), 2.31-2.28 (m, 6H), 2.20-2.13 (m, 1H), 1.92-1.85 (m, 1H), 1.74-1.70 (m, 4H).

The following compounds were prepared analogously to Compound 208:

| Compound | Name |
|---|---|
| 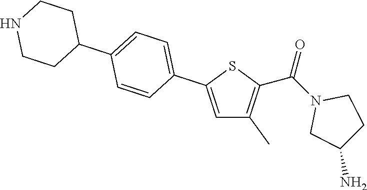 [SMILES: Cc1cc(-c2ccc(C3CCNCC3)cc2)sc1C(=O)N1CC[C@H](N)C1] | (S)-(3-aminopyrrolidin-1-yl){3-methyl-5-[4- (piperidin-4-yl)phenyl]thiophen-2- yl}methanone |
| 205 | |
| 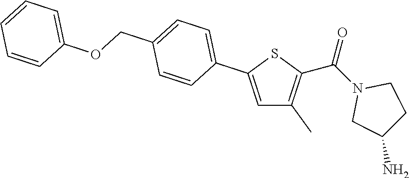 [SMILES: Cc1cc(-c2ccc(COc3ccccc3)cc2)sc1C(=O)N1CC[C@H](N)C1] | (S)-(3-aminopyrrolidin-1-yl){3-methyl-5-[4- (phenoxymethyl)phenyl]thiophen-2- yl}methanone |
| 204 | |
| 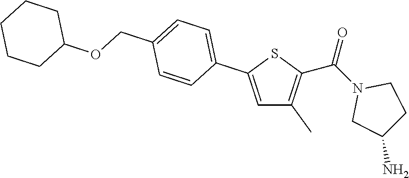 [SMILES: Cc1cc(-c2ccc(COC3CCCCC3)cc2)sc1C(=O)N1CC[C@H](N)C1] | (S)-(3-aminopyrrolidin-1-yl){5-[4- (cyclohexyloxymethyl)phenyl]-3- methylthiophen-2-yl}methanone |
| 203 | |
| 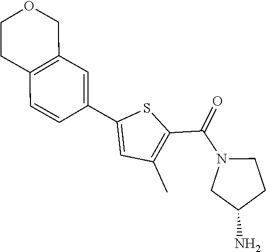 [SMILES: Cc1cc(-c2ccc3c(c2)COCC3)sc1C(=O)N1CC[C@H](N)C1] | (S)-(3-aminopyrrolidin-1-yl)[5-(isochroman- 7-yl)-3-methylthiophen-2-yl]methanone |
| 195 | |
| 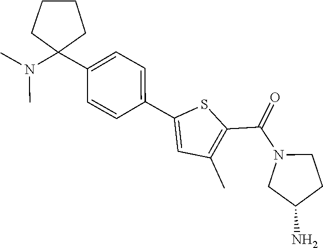 [SMILES: Cc1cc(-c2ccc(C3(N(C)C)CCCC3)cc2)sc1C(=O)N1CC[C@H](N)C1] | (S)-(3-aminopyrrolidin-1-yl){5[4-(1- dimethylaminocyclopentyl)phenyl]-3- methylthiophen-2-yl}methanone |
| 193 | |
| 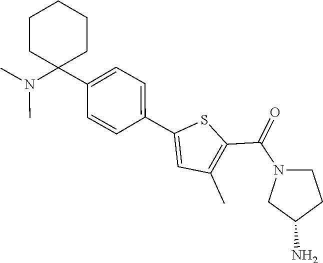 [SMILES: Cc1cc(-c2ccc(C3(N(C)C)CCCCC3)cc2)sc1C(=O)N1CC[C@H](N)C1] | (S)-(3-aminopyrrolidin-1-yl){5-[4-(1- dimethylaminocyclohexyl)phenyl]-3- methylthiophen-2-yl}methanone |
| 192 | |

| Compound | H NMR | |
|---|---|---|
| 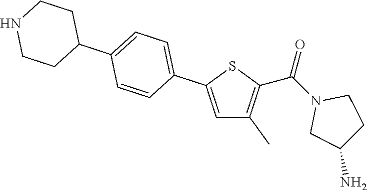 [SMILES: Cc1cc(-c2ccc(C3CCNCC3)cc2)sc1C(=O)N1CC[C@H](N)C1] | ¹H NMR (400 MHz, MeOD) δ 8.27 (s, J = 8.0 Hz, 2H), 7.62 (d, J = 8.0 Hz, 2H), 7.34 (s, 1H), 7.30 (d, J = 8.4 Hz, 2H), 3.84 (s, 1H), 3.72 (m, 2H), 3.56 (m, 2H), 3.39 (d, J = 12.4 Hz, 2H), 3.01 (dd, J = 12.8, 10.0 Hz, 2H), 2.87 (m, 1H), 2.29 (s, 3H), 2.21 (m, 1H), 2.08 (s, 1H), 2.01-1.74 (m, 5H). | 370.2 [M + H]⁺ |
| 205 | | |
| 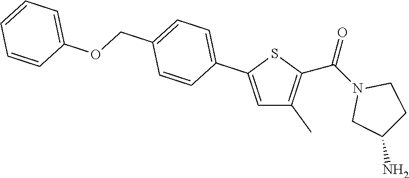 [SMILES: Cc1cc(-c2ccc(COc3ccccc3)cc2)sc1C(=O)N1CC[C@H](N)C1] | ¹H NMR (400 MHz, MeOD) δ 7.67 (d, J = 8.2 Hz, 2H), 7.51 (d, J = 8.2 Hz, 2H), 7.33- 7.26 (m, 3H), 7.03-6.92 (m, 3H), 5.12 (s, 2H), 4.06-3.97 (m, 2H), 3.92-3.68 (m, 3H), 2.47 (td, J = 14.0, 7.5 Hz, 1H), 2.38 (s, 3H), 2.22-2.10 (m, 1H). | 393.2 [M + H]⁺ |
| 204 | | |
| 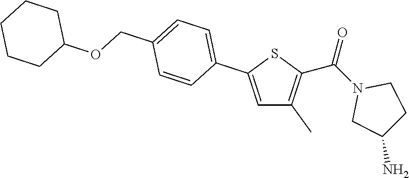 [SMILES: Cc1cc(-c2ccc(COC3CCCCC3)cc2)sc1C(=O)N1CC[C@H](N)C1] | ¹H NMR (400 MHz, MeOD) δ 7.53 (t, J = 6.9 Hz, 2H), 7.29 (d, J = 8.1 Hz, 2H), 7.16 (s, 1H), 4.46 (s, 2H), 3.94-3.85 (m, 2H), 3.79-3.65 (m, 2H), 3.60 (dd, J = 15.0, 6.5 Hz, 1H), 3.31 (td, J = 8.9, 3.8 Hz, 1H), 2.42- 2.30 (m, 1H), 2.29-2.23 (m, 3H), 2.04 (d, J = 5.1 Hz, 1H), 1.87 (d, J = 10.1 Hz, 2H), 1.67 (d, J = 5.2 Hz, 2H), 1.46 (d, J = 6.4 Hz, 1H), 1.32-1.11 (m, 5H). | 399.2 [M + H]⁺ |
| 203 | | |
| 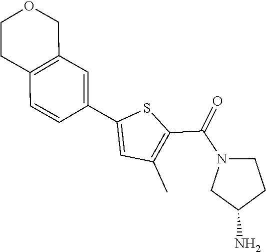 [SMILES: Cc1cc(-c2ccc3c(c2)COCC3)sc1C(=O)N1CC[C@H](N)C1] | ¹H NMR (400 MHz, MeOD) δ 7.43 (d, J = 8.0 Hz, 1H), 7.30 (s, 1H), 7.22 (s, 1H), 7.17 (d, J = 8.0 Hz, 1H), 4.77 (s, 2H), 3.98 (dd, J = 13.0, 7.1 Hz, 4H), 3.90-3.74 (m, 2H), 3.70 (dd, J = 15.1, 6.7 Hz, 1H), 2.85 (t, J = 5.6 Hz, 2H), 2.52-2.39 (m, 1H), 2.38- 2.31 (m, 3H), 2.20-2.07 (m, 1H). | 343.2 [M + H]⁺ |
| 195 | | |
| 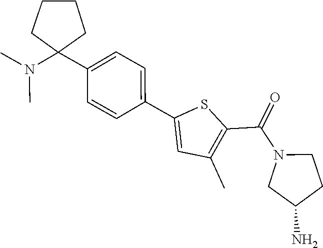 [SMILES: Cc1cc(-c2ccc(C3(N(C)C)CCCC3)cc2)sc1C(=O)N1CC[C@H](N)C1] | ¹H NMR (400 MHz, MeOD) δ 7.84 (t, J = 9.3 Hz, 1H), 7.71 (dd, J = 18.4, 7.2 Hz, 1H), 7.39 (d, J = 9.9 Hz, 1H), 4.02 (s, 1H), 3.82 (d, J = 21.0 Hz, 1H), 3.72 (d, J = 8.3 Hz, 1H), 2.96 (d, J = 11.6 Hz, 1H), 2.70 (d, J = 29.0 Hz, 3H), 2.46 (s, 1H), 2.38 (s, 1H), 2.17 (s, 1H), 1.90 (dd, J = 29.7, 14.3 Hz, 2H), 1.62 (t, J = 9.9 Hz, 1H), 1.47-1.12 (m, 2H). | 398.2 [M + H]⁺ |
| 193 | | |
| 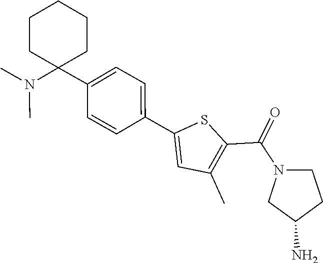 [SMILES: Cc1cc(-c2ccc(C3(N(C)C)CCCCC3)cc2)sc1C(=O)N1CC[C@H](N)C1] | ¹H NMR (400 MHz, MeOD) δ 7.84 (t, J = 9.3 Hz, 1H), 7.71 (dd, J = 18.4, 7.2 Hz, 1H), 7.39 (d, J = 9.9 Hz, 1H), 4.02 (s, 1H), 3.82 (d, J = 21.0 Hz, 1H), 3.72 (d, J = 8.3 Hz, 1H), 2.96 (d, J = 11.6 Hz, 1H), 2.70 (d, J = 29.0 Hz, 3H), 2.46 (s, 1H), 2.38 (s, 1H), 2.17 (s, 1H), 1.90 (dd, J = 29.7, 14.3 Hz, 2H), 1.62 (t, J = 9.9 Hz, 1H), 1.47-1.12 (m, 2H). | 412.2 [M + H]⁺ |
| 192 | | |

The following compounds are prepared analogously to the methods described in this example:

| Compound | Synthetic procedure |
|---|---|
| 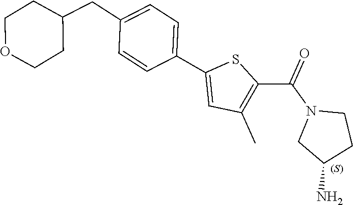 | For step 1, a corresponding aryl boronic ester can be used to synthesize compound 237 |
| 237 | |
| 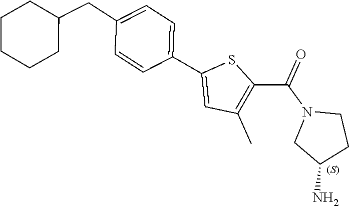 | For step 1, a corresponding aryl boronic ester can be used to synthesize compound 231 |
| 231 | |

### Example 52: Synthesis of (S)-(3-aminopyrrolidin-1-yl){3-methyl-5-[4-(methylphenylaminomethyl)phenyl]thiophen-2-yl}methanone (Compound 207)

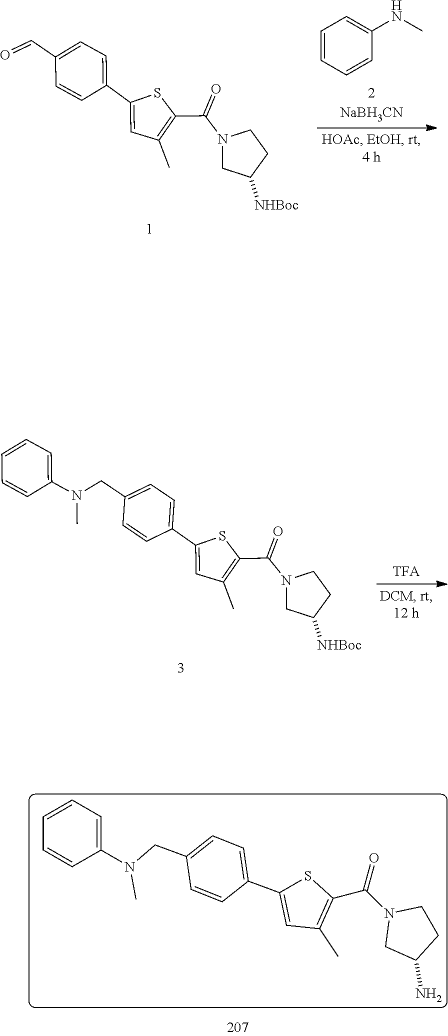

### Step 1 Synthesis of tert-butyl (S)-(1-(3-methyl-5-(4-((methyl(phenyl)amino)methyl)phenyl)thiophene-2-carbonyl)pyrrolidin-3-yl)carbamate

A mixture of tert-butyl N-[(3S)-1-{[5-(4-formylphenyl)-3-methylthiophen-2-yl]carbonyl}pyrrolidin-3-yl]carbamate (200 mg, 0.35 mmol) and aniline (207 mg, 1.4 mmol) in EtOH (2 mL) was degassed and purged with N₂ for 3 times, then the mixture was stirred at 25° C. for 2 h under N₂ atmosphere. After that, NaBH₃CN (60.6 mg, 0.96 mmol) and 2 drops of acetic acid were added to the solution, and the reaction was stirred at 25° C. for another 2 h. LCMS showed that the starting material was consumed and the desired mass was observed. The reaction mixture was partitioned between 10 mL of NH₄C₁ (aq.) and 10 mL of ethyl acetate. The combined organic phase was washed with 4 mL of brine, dried over Na₂SO₄, filtered and concentrated under reduced pressure to give the crude compound (180 mg, 99% yield) as yellow solid. MS (ESI): mass calc'd. for C29H35N3O3S+ [M+H]+ m/z 506.24, found 506.1.

### Step 2 Synthesis of (S)-(3-aminopyrrolidin-1-yl){3-methyl-5-[4-(methylphenylaminomethyl)phenyl]thiophen-2-yl}methanone

A mixture of tert-butyl N-[(3S)-1-({4-methyl-2-[4-(pyrrolidin-1-ylmethyl)phenyl]-1,3-thiazol-5-yl}carbonyl)pyrrolidin-3-yl]carbamate (170 mg, 0.34 mmol) and TFA (471 mg, 3.70 mmol) in dichloromethane (3 mL) was degassed and purged with N₂ for 3 times, then the mixture was stirred at 25° C. for 16 hours under N₂ atmosphere. LCMS showed that the starting material was consumed and the desired mass was observed. The solution was concentrated under reduced pressure to give the residue, which was purified by prep-HPLC to give the desired product (Compound 207, 16.2 mg, 10.6% yield) as white solid. MS (ESI): mass calc'd. for C24H27N3OS+ [M+H]+ m/z 406.19, found 406.1. ¹H NMR (400 MHz, DMSO) δ 7.59 (d, J=8.2 Hz, 2H), 7.31 (s, 1H), 7.26 (d, J=8.4 Hz, 2H), 7.15 (dd, J=8.8, 7.2 Hz, 2H), 6.72 (d, J=8.0 Hz, 2H), 6.61 (t, J=7.2 Hz, 1H), 4.58 (s, 2H), 3.69 (d, J=11.2 Hz, 2H), 3.66-3.48 (m, 2H), 3.40 (d, J=10.0 Hz, 1H), 3.02 (s, 3H), 2.27 (s, 3H), 2.14 (m, 2H), 1.87 (d, J=5.6 Hz, 2H).

The following compounds were prepared analogously to Compound 207:

| Compound | Name |
|---|---|
| 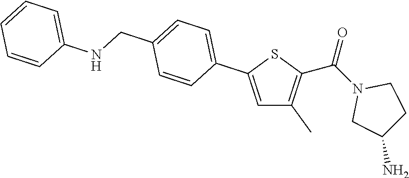 [SMILES: Cc1cc(-c2ccc(CNc3ccccc3)cc2)sc1C(=O)N1CC[C@H](N)C1] | (S)-(3-aminopyrrolidin-1-yl){3-methyl-5-[4- (phenylaminomethyl)phenyl]thiophen-2- yl}methanone |
| 206 | |
| 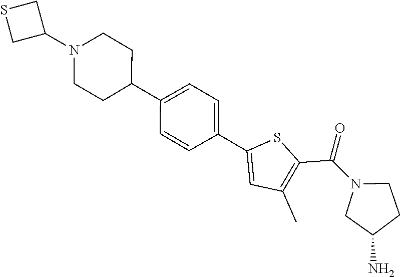 [SMILES: Cc1cc(-c2ccc(C3CCN(C4CSC4)CC3)cc2)sc1C(=O)N1CC[C@H](N)C1] | (S)-(3-aminopyrrolidin-1-yl){3-methyl-5-[4-(1- thietan-3-ylpiperidin-4-yl)phenyl]thiophen-2- yl} methanone |
| 172 | |
| 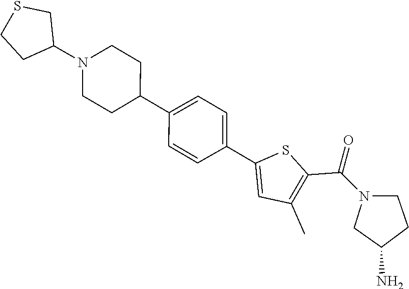 [SMILES: Cc1cc(-c2ccc(C3CCN(C4CCSC4)CC3)cc2)sc1C(=O)N1CC[C@H](N)C1] | (S)-3-aminopyrrolidin-1-yl{3-methyl-5-[4-(1- tetrahydrothiophen-3-ylpiperidin-4- yl)phenyl]thiophen-2-yl}methanone |
| 169 | |

| Compound | H NMR | MS |
|---|---|---|
| 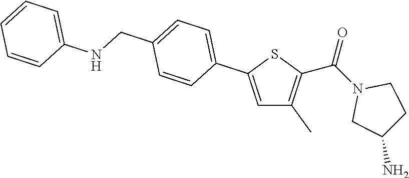 [SMILES: Cc1cc(-c2ccc(CNc3ccccc3)cc2)sc1C(=O)N1CC[C@H](N)C1] | ¹H NMR (400 MHz, DMSO) δ 7.59 (d, J = 8.0 Hz, 2H), 7.41 (d, J = 8.0 Hz, 2H), 7.32 (s, 1H), 7.03 (dd, J = 8.4, 7.2 Hz, 2H), 6.56 (d, J = 7.6 Hz, 2H), 6.51 (t, J = 7.2 Hz, 1H), 6.28 (t, J = 5.6 Hz, 1H), 4.28 (d, J = 5.2 Hz, 2H), 3.77 (s, 1H), 3.75-3.66 (m, 2H), 3.55 (m, 2H), 3.45 (d, J = 9.6 Hz, 2H), 2.28 (s, 3H), 2.18 (m,1H), 1.91 (d, J = 5.2 Hz, 1H). | 392.1 [M + H]⁺ |
| 206 | | |
| 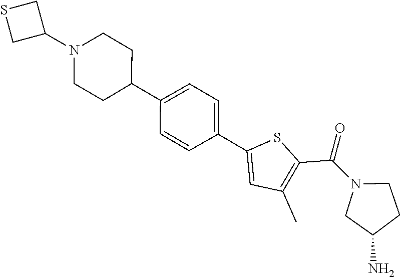 [SMILES: Cc1cc(-c2ccc(C3CCN(C4CSC4)CC3)cc2)sc1C(=O)N1CC[C@H](N)C1] | ¹H NMR (400 MHz, MeOD) δ 7.61 (d, J = 8.3 Hz, 2H), 7.34 (d, J = 8.3 Hz, 2H), 7.24 (s, 1H), 4.60-4.52 (m, 1H), 4.02-3.98 (m, 2H), 3.89-3.69 (m, 5H), 3.59 (d, J = 12.3 Hz, 2H), 3.31- 3.27 (m, 2H), 3.00-2.90 (m, 3H), 2.51- 2.41 (m, 1H), 2.36 (s, 3H), 2.19-2.00 (m, 5H). | 442.1 [M + H]⁺ |
| 172 | | |
| 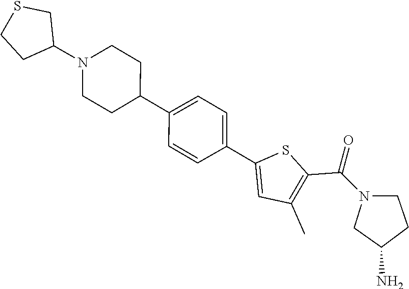 [SMILES: Cc1cc(-c2ccc(C3CCN(C4CCSC4)CC3)cc2)sc1C(=O)N1CC[C@H](N)C1] | ¹H NMR (400 MHz, MeOD) δ 7.62 (d, J = 8.4 Hz, 2H), 7.35 (d, J = 8.4 Hz, 2H), 7.25 (s, 1H), 3.99 (t, J = 7.2 Hz, 2H), 3.91-3.75 (m, 5H), 3.74- 3.67 (m, 1H), 3.28 (t, J = 5.6 Hz, 2H), 3.10 (dd, J = 10.8, 9.2 Hz, IH), 3.06- 2.90 (m, 3H), 2.76-2.64 (m, 1H), 2.45 (dd, J = 13.6, 6.4 Hz, 1H), 2.36 (s, 3H), 2.28-2.06 (m, 7H). | 456.1 [M + H]⁺ |
| 169 | | |

### Example 53: Synthesis of (S)-(3-aminopyrrolidin-1-yl)(3-methyl-5-(4-(((tetrahydro-2H-pyran-4-yl oxy)methyl phenyl thiophen-2-yl methanone (Compound 202)

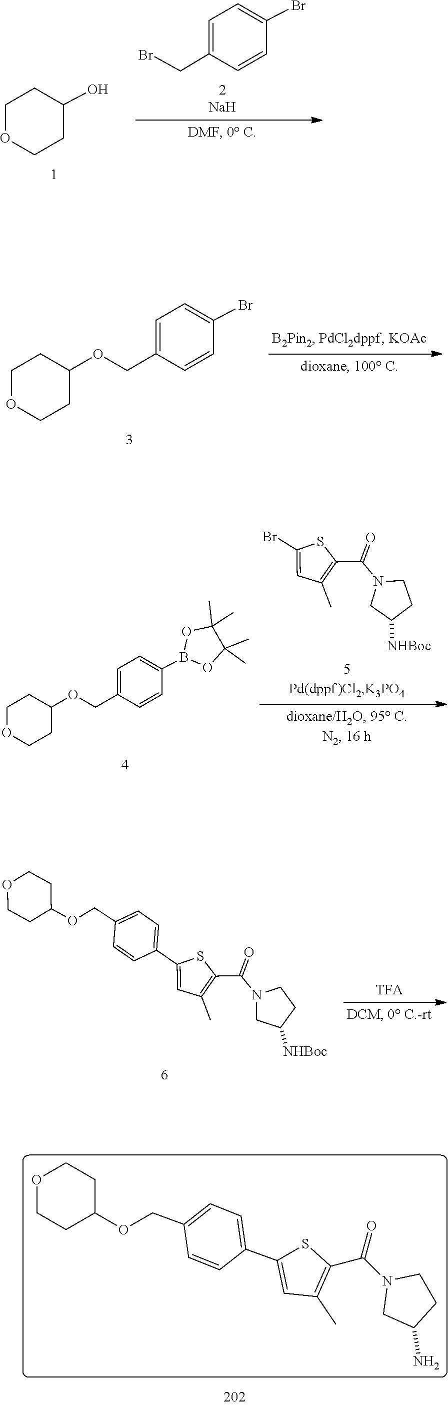

### Step 1 Synthesis of 4-[(4-bromobenzyl)oxy]tetrahydro-2H-pyran

To a solution of NaH (960 mg, w/w=60%, 24.0 mmol) in anhydrous DMF (10 mL) was added oxan-4-ol (2.04 g, 20 mmol) dropwise at 0° C. under nitrogen. Then the reaction was slowly warmed to 25° C. and stirred for 0.5 h. After that, 1-bromo-4-(bromomethyl)benzene (7.5 g, 30 mmol) in DMF (10 mL) was added dropwise to the mixture and the reaction was stirred for another 2 h. After the reaction was completed, the solution was quenched with water and extracted with EtOAc for 3 times. The combined organic phase was washed with brine, dried over Na₂SO₄, filtered and concentrated under reduced pressure to give the residue, which was purified by column chromatography (petroleum ether:ethyl acetate=3:1) to afford 4-(4-bromophenylmethoxy)oxane (4.6 g, 85% yield) as yellowish oil. ¹H NMR (400 MHz, CDCl3) δ 7.48-7.45 (m, 2H), 7.24-7.21 (m, 2H), 4.50 (s, 2H), 3.98-3.93 (m, 2H), 3.60-3.54 (m, 1H), 3.46-3.40 (m, 2H), 1.95-1.89 (m, 2H), 1.71-1.60 (m, 2H).

### Step 2 Synthesis of 4,4,5,5-tetramethyl-2-{4-[(tetrahydro-2H-pyran-4-yloxy)methyl]-Phenyl}-1,3,2-dioxaborolane

To a mixture of 4-(4-bromophenylmethoxy)oxane (2.2 g, 8.1 mmol), B₂Pin₂ (2.26 g, 8.9 mmol), Pd(dppf)Cl₂ (0.18 g, 0.24 mmol) and KOAc (2.38 g, 24.3 mmol) was added DMSO (22 mL), then the suspension was purged with N₂ for 3 times and stirred at 95° C. for 16 h. After the reaction was completed, the mixture was diluted with H₂O and extracted with EtOAc for 3 times. The combined organic phase was dried over Na₂SO₄, filtered and concentrated under reduced pressure to give the crude product, which was purified by column chromatography (dichloromethane:methanol=10:1) to afford 4,4,5,5-tetramethyl-2-(4-((oxan-4-yloxy)methyl)phenyl)-1,3,2-dioxaborolane (1.738 g) as an off-white solid. ¹H NMR (400 MHz, CDCl3) δ 7.79 (d, J=7.9 Hz, 2H), 7.35 (d, J=7.9 Hz, 2H), 4.59 (s, 2H), 3.97-3.94 (m, 2H), 3.60-3.53 (m, 1H), 3.45-3.39 (m, 2H), 1.94-1.90 (m, 2H), 1.70-1.61 (m, 2H), 1.34 (s, 12H).

### Step 3 Synthesis of tert-butyl (S)-(1-(3-methyl-5-(4-(((tetrahydro-2H-pyran-4-yl)oxy)methyl)phenyl)thiophene-2-carbonyl)pyrrolidin-3-yl)carbamate

To a solution of tert-butyl N-{(3S)-1-[(5-bromo-3-methylthiophen-2-yl)carbonyl]pyrrolidin-3-yl}carbamate (200 mg, 0.514 mmol) and 4,4,5,5-tetramethyl-2-[4-(oxan-4-yloxymethyl)phenyl]-1,3,2-dioxaborolane (196.2 mg, 0.616 mmol) in dioxane/H₂O (v/v=8:1, 9 mL) was added K₃PO₄ (327.1 mg, 1.54 mmol) and Pd(dppf)Cl₂·DCM (83.9 mg, 0.1 mmol), then the reaction mixture was stirred at 100° C. under N₂ for 16 h. After the reaction was completed, the solvent was removed under reduced pressure, and the residue was purification by column chromatography (petroleum ether/ethyl acetate=1:1) to afford tert-butyl (S)-1-(3-methyl-5-(4-((tetrahydro-2H-pyran-4-yloxy)methyl)phenyl)thiophene-2-carbonyl)pyrrolidin-3-yl)carbamate (169 mg, 66% yield). MS (ESI): mass calc'd. for C27H36N205S+ [M+1]+ m/z 501.23, found 501.2.

### Step 4 Synthesis of (S)-(3-aminopyrrolidin-1-yl)(3-methyl-5-(4-(((tetrahydro-2H-pyran-4-yl)oxy)methyl)phenyl)thiophen-2-yl)methanone

To a solution of tert-butyl (S)-[1-(3-methyl-5-{4-[(tetrahydro-2H-pyran-4-yloxy)methyl]phenyl}thiophene-2-carbonyl)pyrrolidin-3-yl]carbamate (169 mg, 0.34 mmol) in DCM (5 mL) was added TFA (677.5 mg, 5.94 mmol) at 0° C., then the reaction mixture was slowly warmed to 25° C. and stirred for 12 h. After the reaction was completed, the solvent was removed under reduced pressure to give the residue, which was purified by prep-HPLC to afford (S)-(3-aminopyrrolidin-1-yl)(3-methyl-5-(4-((tetrahydro-2H-pyran-4-yloxy)methyl)phenyl)thiophen-2-yl)methanone (Compound 202, 27.7 mg, 20.3%) as a yellow solid. MS (ESI): mass calc'd. for C22H29N2O3S+ [M+1]+ m/z 401.19, found 401.

¹H NMR (400 MHz, MeOD) δ 7.62 (d, J=8.0 Hz, 2H), 7.40 (d, J=8.0 Hz, 2H), 7.26 (s, 1H), 4.59 (s, 2H), 3.99-3.90 (m, 4H), 3.87-3.72 (m, 2H), 3.69-3.62 (m, 2H), 3.50-3.41 (m, 1H), 2.45-2.35 (m, 4H), 2.14-2.06 (m, 1H), 1.98-1.94 (m, 2H), 1.64-1.55 (m, 1H).

### Example 54: Synthesis of (S)-(3-aminopyrrolidin-1-yl)(3-methyl-5-(1,2,3,4-tetrahydroquinolin-7-yl)thiophen-2-yl)methanone (Compound 197)

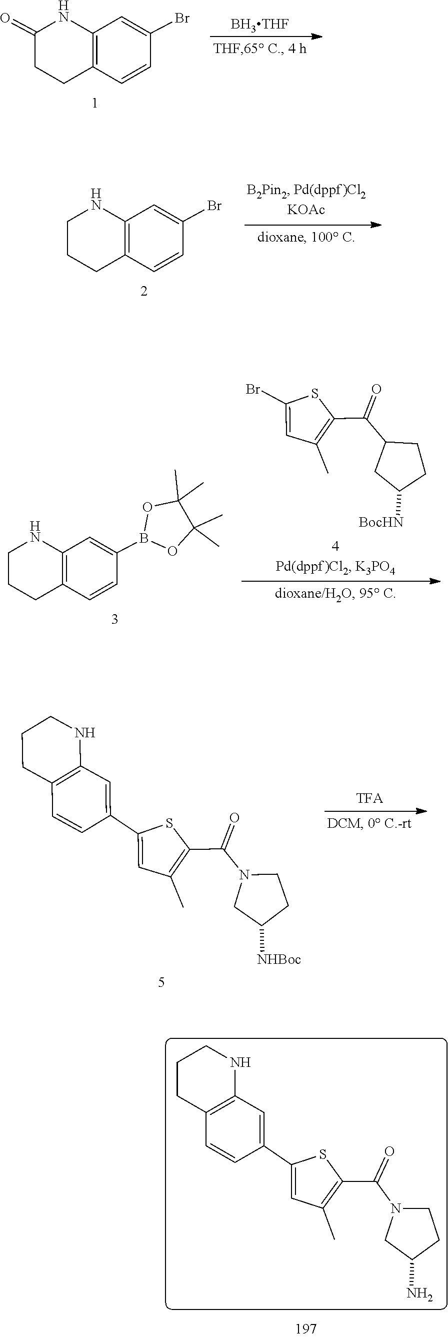

### Step 1 Synthesis of 7-bromo-1,2,3,4-tetrahydroquinoline

To a solution of 7-bromo-3,4-dihydro-1H-quinolin-2-one (2.0 g, 8.8 mmol) in THF (25 mL) was added BIH-THF (1M in THF, 100 mL), then the mixture was stirred 65° C. for 4 h. After the reaction was completed, the mixture was cooled to 0° C. and quenched with MeOH, the resulting mixture was stirred at 60° C. for 1 h. Then the solvent was removed under reduced pressure to give the residue, which was purified by column chromatography to afford 7-bromo-1,2,3,4-tetrahydroquinoline (1.8 g, 96.59% yield) as a colorless oil.

MS (ESI): mass calcd. for C9H10BrN+ [M+1]+ m/z 212.00, found 212.0.

### Step 2 Synthesis of 7-(4,4,5,5-tetramethyl-1,3,2-dioxaborolan-2-yl)-1,2,3,4-tetrahydroquinoline

To a mixture of 7-bromo-1,2,3,4-tetrahydroquinoline (1 g, 4.71 mmol), B₂Pin₂ (1.32 g, 5.18 mmol), Pd(dppf)Cl₂ (0.69 g, 0.94 mmol) and KOAc (1.39 g, 14.14 mmol) was added dioxane (10 mL), then the mixture was stirred at 100° C. for 16 h. After the reaction was completed, the solvent was removed under reduced pressure to give the residue, which was purified by flash column chromatography (dichloromethane:methanol=10:1) to afford 7-(4,4,5,5-tetramethyl-1,3,2-dioxaborolan-2-yl)-1,2,3,4-tetrahydroquinoline (0.8 g, 65% yield) as yellowish solid. MS (ESI): mass calcd. for C15H22BNO2+ [M+1]+ m/z 260.17, found 260.2.

### Step 3 Synthesis of tert-butyl (S)-(1-(3-methyl-5-(1,2,3,4-tetrahydroquinolin-7-yl)thiophene-2-carbonyl)pyrrolidin-3-yl)carbamate

To a mixture of 7-(4,4,5,5-tetramethyl-1,3,2-dioxaborolan-2-yl)-1,2,3,4-tetrahydroquinoline (330 mg, 1.27 mmol), tert-butyl (S)-(1-(5-bromo-3-methylthiophene-2-carbonyl)pyrrolidin-3-yl)carbamate (450 mg, 1.16 mmol), Pd(dppf)Cl₂ (169.2 mg, 0.23 mmol) and K₃PO₄ (736 mg, 3.47 mmol) was added dioxane/H₂O (v:v=8:1, 9 mL), then the suspension was stirred at 95° C. under N₂ for 16 hours. After the reaction was completed, the solvent was removed under reduced pressure to give the residue, which was purified by column chromatography (dichloromethane:methanol=10:1) to afford tert-butyl (S)-(1-(3-methyl-5-(1,2,3,4-tetrahydroquinolin-7-yl)thiophene-2-carbonyl)pyrrolidin-3-yl)carbamate (260 mg, 50% yield) as a brown solid. MS (ESI): mass calcd. for C24H31N3O3S+ [M+1]⁺ m/z 442.21, found 442.2.

### Step 4 Synthesis of (S)-(3-aminopyrrolidin-1-yl)(3-methyl-5-(1,2,3,4-tetrahydroquinolin-7-yl)thiophen-2-yl)methanone

To a solution of tert-butyl (S)-(1-(3-methyl-5-(1,2,3,4-tetrahydroquinolin-7-yl)thiophene-2-carbonyl)pyrrolidin-3-yl)carbamate (130 mg, 0.2944 mmol) in DCM (5 mL) was added TFA (252.6 mg, 2.215 mmol) at 0° C., then the solution was slowly warmed to room temperature and stirred for 16 hours. After the reaction was completed, the solvent and excess of TFA were removed under reduced pressure to give the residue, which was purified by prep-HPLC to afford (S)-(3-aminopyrrolidin-1-yl)(3-methyl-5-(1,2,3,4-tetrahydroquinolin-7-yl)thiophen-2-yl)methanone (Compound 197, 40 mg, 39.78% yield) as a white solid. LCMS (ESI) calcd for C19H24N3OS+ [M+H]+ m/z 342.16, found 342. ¹H NMR (400 MHz, MeOD) δ 7.01 (s, 11H), 6.80 (d, J=7.7 Hz, 1H), 6.70-6.65 (m, 2H), 3.89-3.85 (m, 2H), 3.77-3.62 (m, 2H), 3.59-3.54 (in, 1H), 3.18-3.15 (m, 2H), 2.64 (t, J=6.3 Hz, 2H), 2.35-2.26 (in, 1H), 2.24 (s, 3H), 2.04-1.96 (in, 1H), 1.84-1.78 (in, 2H).

The following compound was prepared analogously to Compound 197:

| Compound | Name | H NMR | MS |
|---|---|---|---|
| | (S)-(3- | ¹H NMR (400 MHz, MeOD) δ 7.60 (d, | 356.0 |
| | aminopyrrolidin- | J = 8.0 Hz, 2H), 7.52 (s, 1H), 7.33 (d, | [M + |
| | 1-yl)(3-methyl-5- | J = 8.0 Hz, 2H), 7.29 (s, 1H), 4.50 (s, | H]⁺ |
| | (2-methyl-1,2,3,4- | 2H), 4.08-3.95 (m, 2H), 3.88-3.56 | |
| | tetrahydroisoquinolin- | (m, 5H), 3.24 (s, 4H), 3.08 (s, 3H), | |
| | 7-yl)thiophen-2- | 2.53-2.41 (m, 1H), 2.36 (s, 3H), 2.22- | |
| | yl)methanone | 2.09 (m, 1H). | |

The following compounds are prepared analogously to the methods described in this example:

| Compound | Synthesis |
|---|---|
| 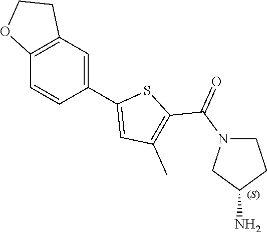 | For step 2, a corresponding bromo reagent can be used to synthesize compound 210 |
| 210 | |
| 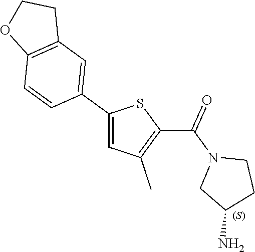 | For step 2, a corresponding bromo reagent can be used to synthesize compound 213 |
| 213 | |
| 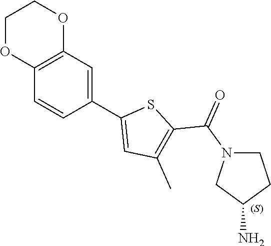 | For step 2, a corresponding bromo reagent can be used to synthesize compound 216 |
| 216 | |
| 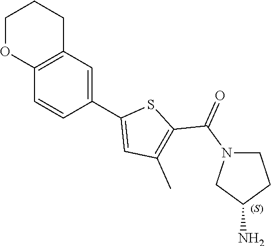 | For step 2, a corresponding bromo reagent can be used to synthesize compound 229 |
| 229 | |
| 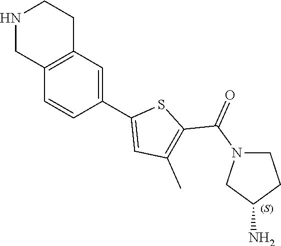 | For step 2, a corresponding bromo reagent can be used to synthesize compound 240 |
| 240 | |
| 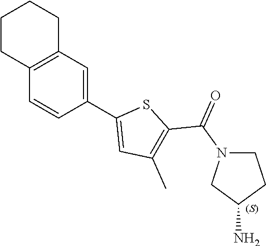 | For step 2, a corresponding bromo reagent can be used to synthesize compound 247 |
| 247 | |
| 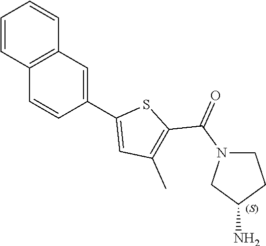 | For step 2, a corresponding bromo reagent can be used to synthesize compound 246 |
| 246 | |

### Example 55: Synthesis of (S)-(3-aminopyrrolidin-1-yl)(3-methyl-5-(1-methyl-1,2,3,4-tetrahydroquinolin-7-yl)thiophen-2-yl)methanone (Compound 196)

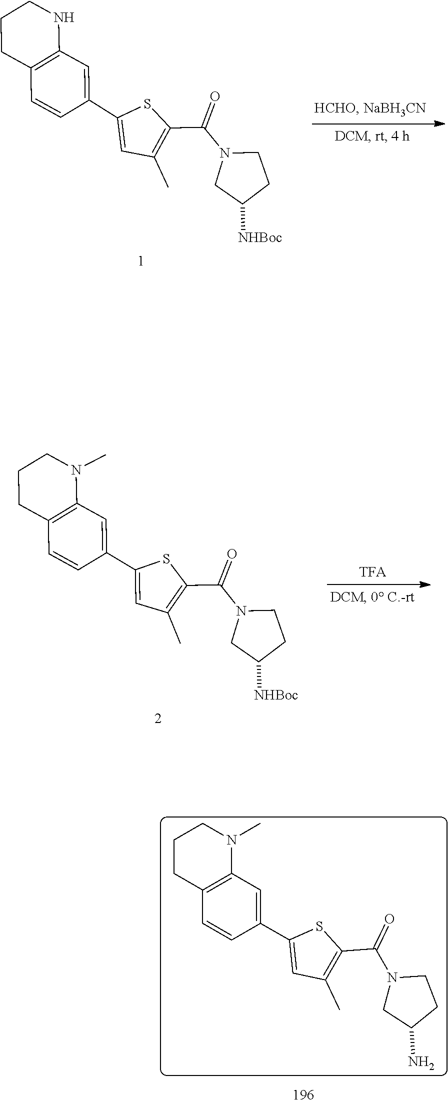 [SMILES: Cc1cc(-c2ccc3c(c2)NCCC3)sc1C(=O)N1CC[C@H](NC(=O)OC(C)(C)C)C1 ; Cc1cc(-c2ccc3c(c2)N(C)CCC3)sc1C(=O)N1CC[C@H](NC(=O)OC(C)(C)C)C1 ; Cc1cc(-c2ccc3c(c2)N(C)CCC3)sc1C(=O)N1CC[C@H](N)C1]

### Step 1 Synthesis of tert-butyl (S)-(1-(3-methyl-5-(1-methyl-1,2,3,4-tetrahydroquinolin-7-yl)thiophene-2-carbonyl)pyrrolidin-3-yl)carbamate

To a solution of (S)-(1-(3-methyl-5-(1,2,3,4-tetrahydroquinolin-7-yl)thiophene-2-carbonyl)pyrrolidin-3-yl)carbamate (130 mg, 0.29 mmol) in DCM (25 mL) was added HCHO (0.5 mL, 37% in H₂O) and NaBH₃CN (56 mg, 0.88 mmol), and the mixture was stirred room temperature for 4 h. After the reaction was completed, the solvent was removed under reduced pressure to give the residue, which was purified by column chromatography (dichloromethane:methanol=10:1) to afford tert-butyl (S)-(1-(3-methyl-5-(1-methyl-1,2,3,4-tetrahydroquinolin-7-yl)thiophene-2-carbonyl)pyrrolidin-3-yl)carbamate (70 mg, 52.1% yield) as a yellow solid. LCMS (ESI) calc'd for C25H33N3O3S+ [M+H]+ m/z 456.22, found 456.2.

### Step 2 Synthesis of (S)-(3-aminopyrrolidin-1-yl)(3-methyl-5-(1-methyl-1,2,3,4-tetrahydroquinolin-7-yl)thiophen-2-yl)methanone

To a solution of tert-butyl (S)-(1-(3-methyl-5-(1-methyl-1,2,3,4-tetrahydroquinolin-7-yl)thiophene-2-carbonyl)pyrrolidin-3-yl)carbamate (70 mg, 0.1536 mmol) in DCM (5 mL) was added TFA (252.6 mg, 2.215 mmol) at 0° C., then the mixture was slowly warmed to room temperature and stirred for 16 hours. After the reaction was completed, the solvent and excess of TFA were removed under reduced pressure to give the residue, which was purified by prep-HPLC to afford (S)-(3-aminopyrrolidin-1-yl)(3-methyl-5-(1-methyl-1,2,3,4-tetrahydroquinolin-7-yl)thiophen-2-yl)methanone (Compound 196, 30 mg, 32.04% yield) as a white solid. LCMS (ESI) calc'd for C20H26N3OS+ [M+H]+ m/z 356.18, found 356. ¹H NMR (400 MHz, MeOD) δ 7.06 (s, 1H), 6.83 (d, J=7.6 Hz, 1H), 6.73-6.69 (m, 2H), 3.89-3.85 (m, 2H), 3.78-3.66 (m, 2H), 3.56-3.54 (m, 1H), 3.17-3.14 (m, 2H), 2.83 (s, 3H), 2.65 (t, J=6.3 Hz, 2H), 2.33-2.27 (m, 1H), 2.25 (s, 3H), 2.02-1.95 (m, 1H), 1.91-1.85 (m, 2H).

### Example 56: Synthesis of (S)-(3-aminopyrrolidin-1-yl)(5-(chroman-7-yl)-3-methylthiophen-2-yl)methanone (Compound 194)

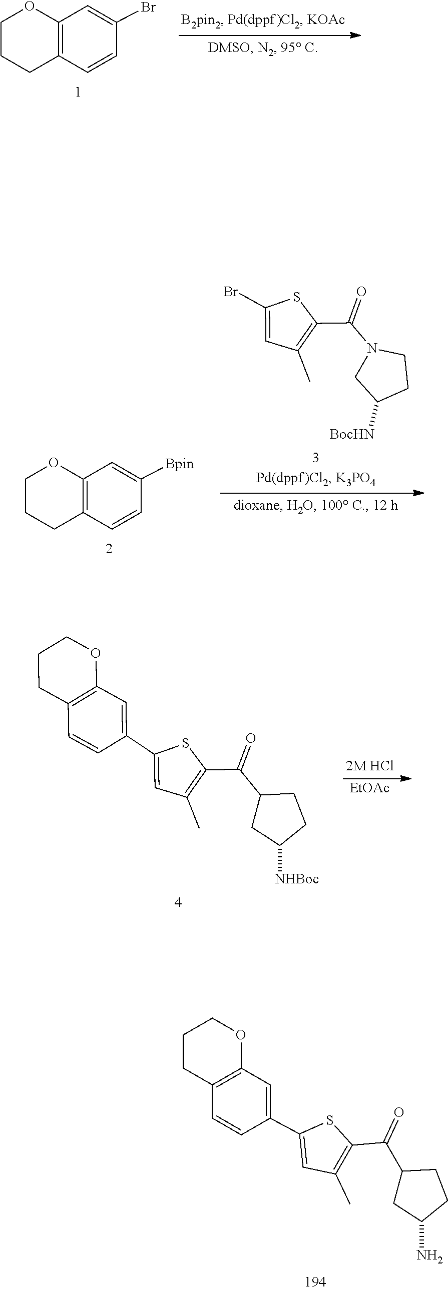

### Step 1 Synthesis of 2-(3,4-dihydro-2H-1-benzopyran-7-yl)-4,4,5,5-tetramethyl-1,3,2-dioxaborolane

To a mixture of 7-bromo-3,4-dihydro-2H-1-benzopyran (500 mg, 2.35 mmol) was added 4,4,5,5-tetramethyl-2-(4,4,5,5-tetramethyl-1,3,2-dioxaborolan-2-yl)-1,3,2-dioxaborolane (596 mg, 2.35 mmol), Pd(dppf)Cl₂ (343 mg, 0.47 mmol) and KOAc (691 mg, 7 mmol) was added dioxane (10 mL) under nitrogen, then the mixture was stirred at 100° C. for 8 h. After that, the suspension was filtered and concentrated under reduced pressure to give the residue, which was purified by flash chromatography (petroleum ether:ethyl acetate=3:1) to afford 2-(3,4-dihydro-2H-1-benzopyran-7-yl)-4,4,5,5-tetramethyl-1,3,2-dioxaborolane (400 mg, 62.25% yield) as yellow oil. LCMS (ESI) calc'd for C15H21BO3+[M+H]+ m/z 261.14, found 261.2.

### Step 2 Synthesis of tert-butyl N-[(3S)-1-{[5-(3,4-dihydro-2H-1-benzopyran-7-yl)-3-methylthiophen-2-yl]carbonyl}pyrrolidin-3-yl]carbamate

To a solution of tert-butyl N-[(3S)-1-[(5-bromo-3-methylthiophen-2-yl)carbonyl]pyrrolidin-3-yl]carbamate (200 mg, 0.5 mmol) in dioxane/H₂O (v:v=8:1, 9 mL) was added 2-(3,4-dihydro-2H-1-benzopyran-7-yl)-4,4,5,5-tetramethyl-1,3,2-dioxaborolane (133.6 mg, 0.5 mmol), K₃PO₄ (327 mg, 1.5 mmol) and Pd(dppf)Cl₂ (75 mg, 0.1 mmol) under nitrogen, then the mixture was stirred at 95° C. for 16 h. After that, the suspension was filtered and concentrated under reduced pressure to give the residue, which was purified by flash chromatography (petroleum ether:ethyl acetate=5:4) to afford tert-butyl N-[(3S)-1-{[5-(3,4-dihydro-2H-1-benzopyran-7-yl)-3-methylthiophen-2-yl]carbonyl}pyrrolidin-3-yl]carbamate (90 mg, 37.6% yield) as yellow oil. LCMS (ESI) calc'd for C24H30N2O4S+ [M+H]+ m/z 443.19, found 443.2.

### Step 2 Synthesis of (S)-(3-aminopyrrolidin-1-yl)(5-(chroman-7-yl)-3-methylthiophen-2-yl)methanone

A solution of tert-butyl N-[(3S)-1-{[5-(3,4-dihydro-2H-1-benzopyran-7-yl)-3-methylthiophen-2-yl]carbonyl}pyrrolidin-3-yl]carbamate (90 mg, 0.20 mmol) in 2M HCl in EA (3 mL) was prepared under nitrogen, then the mixture was stirred at rt for 6 h. LCMS showed that the starting material was consumed and the desired mass was detected. Then the solution was concentrated under reduced pressure to give (3S)-1-{[5-(3,4-dihydro-2H-1-benzopyran-7-yl)-3-methylthiophen-2-yl]carbonyl}pyrrolidin-3-amine (Compound 194, 89.1 mg, 111% yield) as a yellow solid. LCMS (ESI) calc'd for C19H22N2O2S+ [M+H]+ m/z 343.14, found 343.2.

¹H NMR (400 MHz, MeOD) δ 7.07 (s, 1H), 7.01-6.94 (m, 2H), 6.87 (t, J=4.4 Hz, 1H), 4.12-4.05 (m, 2H), 3.94-3.85 (m, 2H), 3.79-3.65 (m, 2H), 3.60 (dd, J=14.9, 6.3 Hz, 1H), 2.69 (t, J=6.4 Hz, 2H), 2.35 (dd, J=13.1, 5.9 Hz, 1H), 2.26 (d, J=8.3 Hz, 4H), 2.04 (d, J=4.9 Hz, 1H), 1.94-1.85 (m, 2H).

The following compounds were prepared analogously to Compound 194:

| Compound | Name | H NMR | MS |
|---|---|---|---|
| 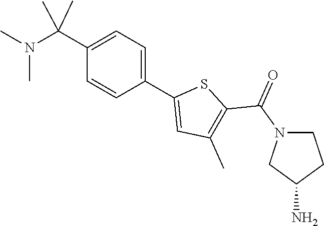 [SMILES: Cc1cc(-c2ccc(C(C)(C)N(C)C)cc2)sc1C(=O)N1CC[C@H](N)C1] | (S)-(3- aminopyrrolidin-1- yl)(5-(4-(2- (dimethylamino) propan-2-yl)phenyl)-3- methylthiophen-2- yl)methanone | ¹H NMR (400 MHz, MeOD) δ 7.74 (dd, J = 24.2, 7.6 Hz, 4H), 7.35 (s, 1H), 3.98-3.68 (m, 5H), 2.69 (s, 6H), 2.42- 2.36 (m, 4H), 2.14 (s, 1H), 1.81 (s, 6H). | 372.2 [M + H] ⁺ |
| 190 | | | |
| 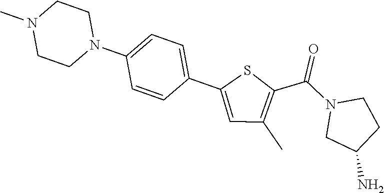 [SMILES: Cc1cc(-c2ccc(N3CCN(C)CC3)cc2)sc1C(=O)N1CC[C@H](N)C1] | (S)-(3- aminopyrrolidin-1- yl)(3-methyl-5-(4- (4-methylpiperazin- 1- yl)phenyl)thiophen- 2-yl)methanone | ¹H NMR (400 MHz, MeOD) δ 7.54 (d, J = 8.4 Hz, 2H), 7.12 (s, 1H), 7.02 (d, J = 8.4 Hz, 2H), 3.97 (d, J = 7.6 Hz, 2H), 3.90-3.64 (m, 3H), 3.43 (s, 4H), 3.14 (s, 4H), 2.74 (s, 3H), 2.49-2.27 (m, 4H), 2.12 (d, J = 4.8 Hz, 1H). | 385.1 [M + H] ⁺ |
| 185 | | | |

### Example 57: Synthesis of (S)-(3-aminopyrrolidin-1-yl)(3-methyl-5-(2-methyl-1,2,3,4-tetrahydroisoquinolin-6-yl)thiophen-2-yl)methanone (Compound 191)

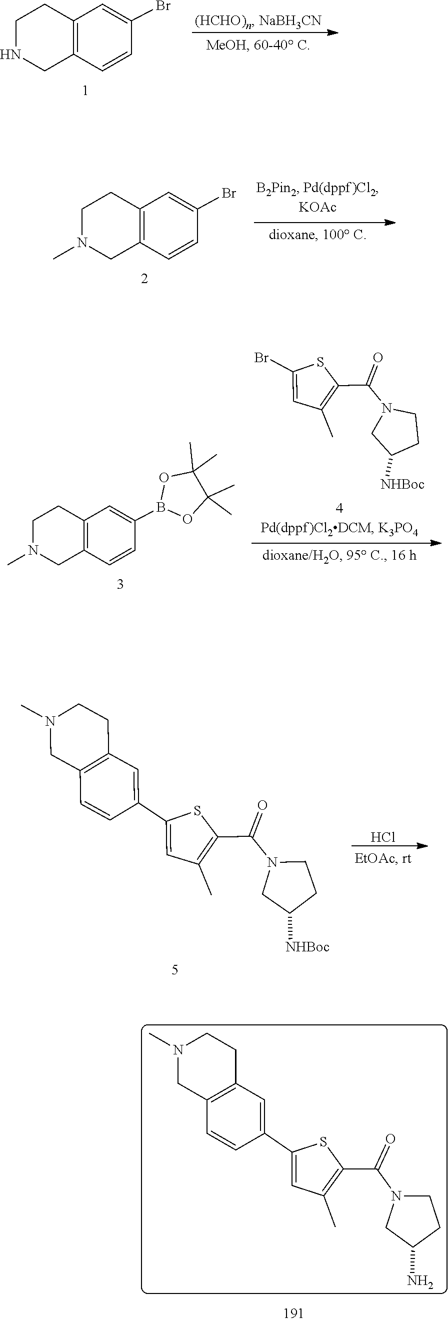

### Step 1 Synthesis of 6-bromo-2-methyl-1,2,3,4-tetrahydroisoquinoline

A solution of paraformaldehyde (350 mg, 3.63 mmol) in MeOH (1 mL) was stirred at 60° C. under N₂ for 1 hour and cooled to 40° C., then 1 drop of AcOH and 6-bromo-1,2,3,4-tetrahydroisoquinoline (350 mg, 1.65 mmol) were added into the system, and the mixture was stirred at 40° C. for another 1 hour. After the reaction was completed, the solvent was removed under reduced pressure to give the residue, which was purified by column chromatography to afford 356 mg 6-bromo-2-methyl-1,2,3,4-tetrahydroisoquinoline as brown oil.

LCMS (ESI) calc'd for C10H12BrN+ [M+H]+ m/z 226.02. found 226.0.

### Step 2 Synthesis of 2-methyl-6-(4,4,5,5-tetramethyl-1,3,2-dioxaborolan-2-yl)-1,2,3,4-tetrahydroisoquinoline

To a solution of 6-bromo-2-methyl-1,2,3,4-tetrahydroisoquinoline (356 mg, 1.5744 mmol), B₂Pin₂ (444.7 mg, 1.7513 mmol) and KOAc (468.7 mg, 4.7763 mmol) in 4 mL dioxane was added Pd(dppf)Cl₂ (35.0 mg, 0.0478 mmol), the mixture was stirred at 100° C. under N₂ for 16 hours. After the reaction, the solvent was removed under reduced pressure, the residue was purified by column chromatography (hexanes/ethyl acetate=1:1) to afford 2-methyl-6-(4,4,5,5-tetramethyl-1,3,2-dioxaborolan-2-yl)-1,2,3,4-tetrahydroisoquinoline (212 mg, 46% yield) as brown oil. MS (ESI): mass calc'd. for C16H25BNO2+ [M+H]+ m/z 274.20, found 274.

### Step 3 Synthesis of tert-butyl (S)-(1-(3-methyl-5-(2-methyl-1, 2, 3,4-tetrahydroisoquinolin-6-yl)thiophene-2-carbonyl)pyrrolidin-3-yl)carbamate

To a solution of tert-butyl (S)-(1-(5-bromo-3-methylthiophene-2-carbonyl)pyrrolidin-3-yl)carbamate (246.2 mg, 0.6325 mmol), 2-methyl-6-(4,4,5,5-tetramethyl-1,3,2-dioxaborolan-2-yl)-1,2,3,4-tetrahydroisoquinoline (144 mg, 0.5271 mmol) and K₃PO₄ (335.7 mg, 1.5813 mmol) in dioxane/H₂O (9 mL, v:v=8:1) was added Pd(dppf)Cl₂ (77.1 mg, 0.1054 mmol), then the mixture was stirred at 95° C. under N₂ for 16 hours. After the reaction was completed, the solvent was removed under reduced pressure to give the residue, which was purified by column chromatography to afford tert-butyl (S)-(1-(3-methyl-5-(2-methyl-1,2,3,4-tetrahydroisoquinolin-6-yl)thiophene-2-carbonyl)pyrrolidin-3-yl)carbamate (68 mg, 27%) as yellow oil.

MS (ESI): mass calc'd. for C25H33N3O3S+ [M+H]+ m/z 456.22, found 456.2.

### Step 4 Synthesis of (S)-(3-aminopyrrolidin-1-yl)(3-methyl-5-(2-methyl-1,2,3,4-tetrahydroisoquinolin-6-yl)thiophen-2-yl)methanone

To a solution of tert-butyl (S)-(1-(3-methyl-5-(2-methyl-1,2,3,4-tetrahydroisoquinolin-6-yl)thiophene-2-carbonyl)pyrrolidin-3-yl)carbamate (68 mg, 0.1492 mmol) in EtOAc (2 mL) was added HCl (4 mL, 2M in EtOAc) slowly at 25° C., then the mixture was stirred at 25° C. for 16 hours. After the reaction was completed, the solvent was removed under reduced pressure to give the residue, which was purified by prep-HPLC to afford (S)-(3-aminopyrrolidin-1-yl)(3-methyl-5-(2-methyl-1,2,3,4-tetrahydroisoquinolin-6-yl)thiophen-2-yl)methanone (Compound 191, 30 mg, 55%) as a yellow solid. MS (ESI): mass calc'd. for C20H26N3OS+ [M+H]+ m/z 356.18, found 356. ¹H NMR (400 MHz, MeOD) δ 7.57 (d, J=5.4 Hz, 2H), 7.30 (s, 1H), 7.25 (d, J=8.6 Hz, 1H), 4.46 (d, J=38.9 Hz, 2H), 3.98-3.96 (m, 2H), 3.87-3.67 (m, 4H), 3.48 (s, 1H), 3.25 (s, 2H), 3.07 (s, 3H), 2.49-2.40 (m, 1H), 2.35 (s, 3H), 2.19-2.10 (m, 1H).

The following compounds were prepared analogously to Compound 191:

| Compound | Name |
|---|---|
| 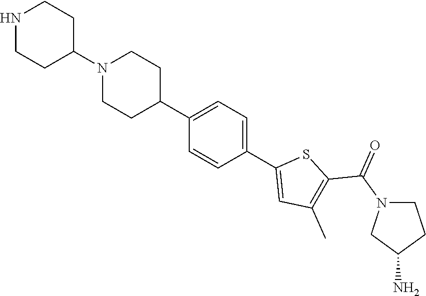 [SMILES: Cc1cc(-c2ccc(C3CCN(C4CCNCC4)CC3)cc2)sc1C(=O)N1CC[C@H](N)C1] | (S)-(5-(4-([1,4′-bipiperidin]-4- yl)phenyl)-3-methylthiophen- 2-yl)(3-aminopyrrolidin-1- yl)methanone |
| 177 | |
| 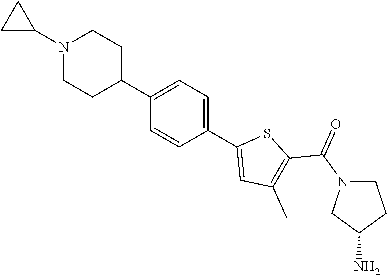 [SMILES: Cc1cc(-c2ccc(C3CCN(C4CC4)CC3)cc2)sc1C(=O)N1CC[C@H](N)C1] | (S)-(3-aminopyrrolidin-1- yl)(5-(4-(1- cyclopropylpiperidin-4- yl)phenyl)-3-methylthiophen- 2-yl)methanone |
| 175 | |
| 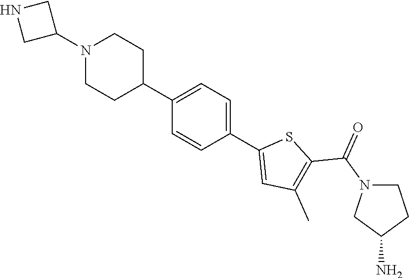 [SMILES: Cc1cc(-c2ccc(C3CCN(C4CNC4)CC3)cc2)sc1C(=O)N1CC[C@H](N)C1] | (S)-(3-aminopyrrolidin-1- yl)(5-(4-(1-(azetidin-3- yl)piperidin-4-yl)phenyl)-3- methylthiophen-2- yl)methanone |
| 174 | |
| 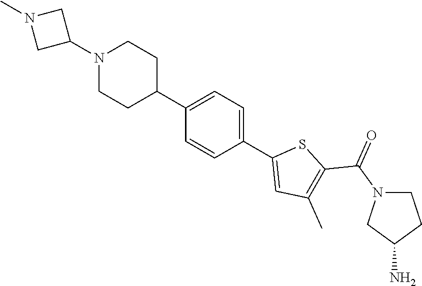 [SMILES: Cc1cc(-c2ccc(C3CCN(C4CN(C)C4)CC3)cc2)sc1C(=O)N1CC[C@H](N)C1] | (S)-(3-aminopyrrolidin-1- yl)(3-methyl-5-(4-(1-(1- methylazetidin-3-yl)piperidin- 4-yl)phenyl)thiophen-2- yl)methanone |
| 173 | |
| 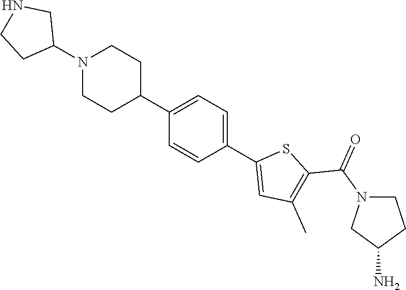 [SMILES: Cc1cc(-c2ccc(C3CCN(C4CCNC4)CC3)cc2)sc1C(=O)N1CC[C@H](N)C1] | ((S)-3-aminopyrrolidin-1- yl)(3-methyl-5-(4-(1- (pyrrolidin-3-yl)piperidin-4- yl)phenyl)thiophen-2- yl)methanone |
| 171 | |
| 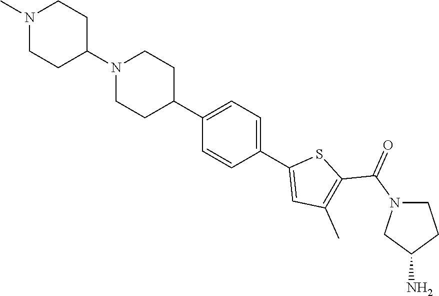 [SMILES: Cc1cc(-c2ccc(C3CCN(C4CCN(C)CC4)CC3)cc2)sc1C(=O)N1CC[C@H](N)C1] | (S)-(3-aminopyrrolidin-1- yl)(3-methyl-5-(4-(1′-methyl- [1,4′-bipiperidin]-4- yl)phenyl)thiophen-2- yl)methanone |
| 168 | |

| Compound | H NMR | MS |
|---|---|---|
| 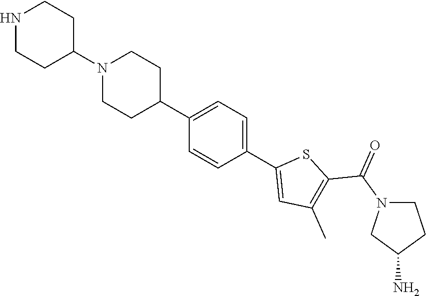 [SMILES: Cc1cc(-c2ccc(C3CCN(C4CCNCC4)CC3)cc2)sc1C(=O)N1CC[C@H](N)C1] | ¹H NMR (400 MHz, MeOD) δ 7.51 (d, J = 8.4 Hz, 2H), 7.24 (d, J = 8.4 Hz, 2H), 7.14 (s, 1H), 3.95-3.85 (m, 2H), 3.80-3.56 (m, 3H), 3.53- 3.38 (m, 4H), 3.29 (t, J = 11.6 Hz, 1H), 3.08- 2.88 (m, 4H), 2.83-2.69 (m, 1H), 2.51-2.31 (m, 1H), 2.27 (s, 5H), 2.13-1.82 (m, 7H). | 453.1 [M + H]⁺ |
| 177 | | |
| 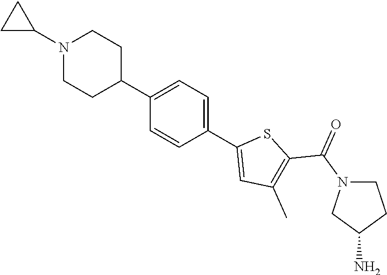 [SMILES: Cc1cc(-c2ccc(C3CCN(C4CC4)CC3)cc2)sc1C(=O)N1CC[C@H](N)C1] | ¹H NMR (400 MHz, ) δ 7.61 (d, J = 8.2 Hz, 2H), 7.34 (d, J = 8.2 Hz, 2H), 7.25 (s, 1H), 4.01- 3.98 (m, 2H), 3.86-3.68 (m, 5H), 3.39-3.33 (m, 2H), 3.01-2.93 (m, 1H), 2.86-2.83 (m, 1H), 2.51-2.41 (m, 1H), 2.36 (s, 3H), 2.19- 2.01 (m, 5H), 1.14 (q, J = 6.9 Hz, 2H), 1.07- 0.96 (m, 2H). | 410.1 [M + H]⁺ |
| 175 | | |
| 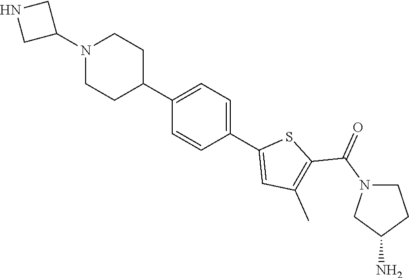 [SMILES: Cc1cc(-c2ccc(C3CCN(C4CNC4)CC3)cc2)sc1C(=O)N1CC[C@H](N)C1] | ¹H NMR (400 MHz, MeOD) δ 7.62 (d, J = 7.2 Hz, 2H), 7.36 (d, J = 7.6 Hz, 2H), 7.25 (s, 1H), 4.75 (s, 2H), 4.42 (s, 3H), 4.00 (d, J = 7.2 Hz, 2H), 3.89-3.53 (m, 5H), 3.04 (d, J = 49.6 Hz, 3H), 2.45 (s, 1H), 2.36 (s, 3H), 2.19 (m, 5H). | 425.1 [M + H]⁺ |
| 174 | | |
| 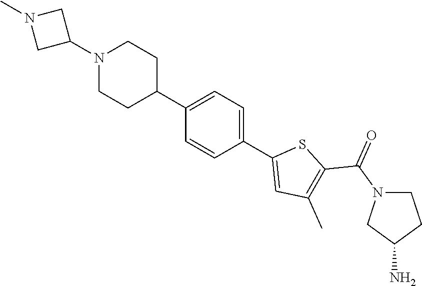 [SMILES: Cc1cc(-c2ccc(C3CCN(C4CN(C)C4)CC3)cc2)sc1C(=O)N1CC[C@H](N)C1] | ¹H NMR (400 MHz, MeOD) δ 7.56 (d, J = 8.4 Hz, 2H), 7.28 (d, J = 8.4 Hz, 2H), 7.19 (s, 1H), 3.77 (m, 2H), 3.67-3.57 (m, 4H), 3.35 (d, J = 5.2 Hz, 1H), 3.07-2.98 (m, 3H), 2.91 (d, J = 11.2 Hz, 2H), 2.58 (m, 1H), 2.40 (s, 3H), 2.32 (s, 3H), 2.20 (s, 1H), 2.07-1.92 (m, 2H), 1.90- 1.69 (m, 5H). | 439.1 [M + H]⁺ |
| 173 | | |
| 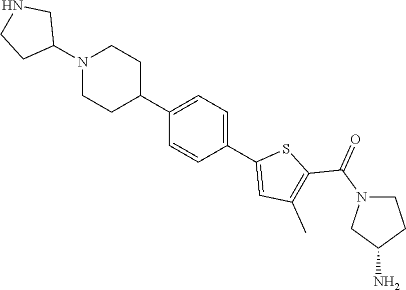 [SMILES: Cc1cc(-c2ccc(C3CCN(C4CCNC4)CC3)cc2)sc1C(=O)N1CC[C@H](N)C1] | ¹H NMR (400 MHz, MeOD) δ 7.57 (d, J = 8.0 Hz, 2H), 7.29 (d, J = 8.0 Hz, 2H), 7.19 (s, 1H), 3.82-3.73 (m, 2H), 3.67-3.52 (m, 2H), 3.38- 3.32 (m, 1H), 3.26-3.15 (m, 2H), 3.14-2.95 (m, 3H), 2.94-2.76 (m, 2H), 2.67-2.56 (m, 1H), 2.33 (s, 3H), 2.27-2.06 (m, 4H), 1.92- 1.70 (m, 6H). | 439.7 [M + H]⁺ |
| 171 | | |
| 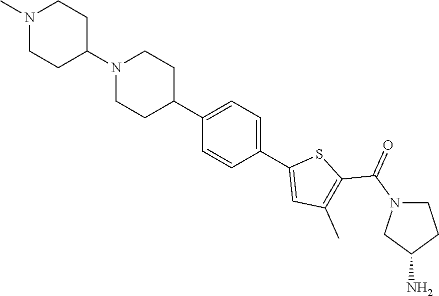 [SMILES: Cc1cc(-c2ccc(C3CCN(C4CCN(C)CC4)CC3)cc2)sc1C(=O)N1CC[C@H](N)C1] | ¹H NMR (400 MHz, MeOD) δ 7.56 (d, J = 8.4 Hz, 2H), 7.28 (d, J = 8.4 Hz, 2H), 7.19 (s, 1H), 3.77 (m, 2H), 3.68-3.58 (m, 2H), 3.34 (d, J = 5.2 Hz, 1H), 3.11 (d, J = 11.6 Hz, 2H), 2.98 (d, J = 11.6 Hz, 2H), 2.58 (m, 1H), 2.45-2.25 (m, 9H), 2.16 (s, 1H), 2.08 (t, J = 11.6 Hz, 2H), 1.97-1.58 (m, 9H). | 467.2 [M + H]⁺ |
| 168 | | |

### Example 58: Synthesis of ((S)-3-aminopyrrolidin-1-yl)(5-(1-(dimethylamino)-2,3-dihydro-1H-inden-5-yl)-3-methylthiophen-2-yl)methanone (Compound 188)

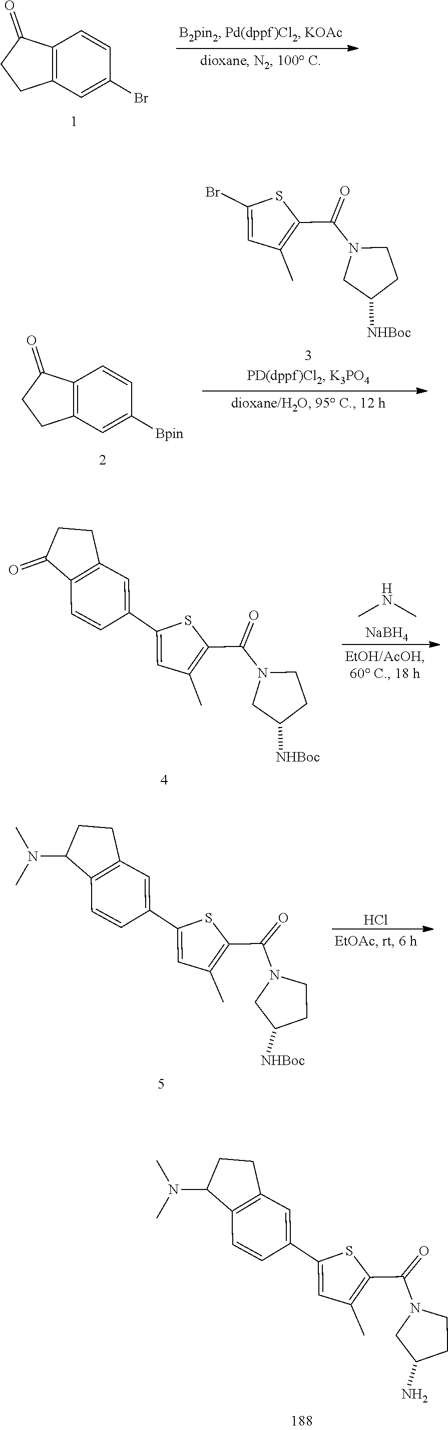

### Step 1 Synthesis of 5-(4,4,5,5-tetramethyl-1,3,2-dioxaborolan-2-yl)-2,3-dihydroinden-1-one

To a solution of 5-5-bromo-2,3-dihydroinden-1-one (500 mg, 2.37 mmol) in dioxane (10 mL) was added 4,4,5,5-tetramethyl-2-(4,4,5,5-tetramethyl-1,3,2-dioxaborolan-2-yl)-1,3,2-dioxaborolane (601 mg, 2.37 mmol), potassium acetate (697 mg, 7.1 mmol) and Pd(dppf)Cl₂ (347 mg, 0.47 mmol). Then the mixture was stirred at 95° C. for 16 h. After that, the mixture was filtered and concentrated under reduced pressure to give the residue, which was purified by flash chromatography (petroleum ether:ethyl acetate=9:1) to afford 5-(4,4,5,5-tetramethyl-1,3,2-dioxaborolan-2-yl)-2,3-dihydroinden-1-one (510 mg, 79% yield) as a yellow solid.

LCMS (ESI) calc'd for C15H19BO3+ [M+H]+ m/z 259.14, found 259.2.

### Step 2 Synthesis of (S)-(1-(3-methyl-5-(1-oxo-2,3-dihydro-1H-inden-5-yl)thiophene-2-carbonyl)pyrrolidin-3-yl)carbamate

To a solution of tert-butyl N-[(3S)-1-[(5-bromo-3-methylthiophen-2-yl)carbonyl]pyrrolidin-3-yl]carbamate (390 mg, 1 mmol) in dioxane/H₂O (v:v=8:1, 18 mL) was added 5-(4,4,5,5-tetramethyl-1,3,2-dioxaborolan-2-yl)-2,3-dihydroinden-1-one (258.6 mg, 1 mmol), K₃PO₄ (638 mg, 3 mmol) and Pd(dppf)Cl₂ (146 mg, 0.2 mmol), then the mixture was stirred at 95° C. for 16 h. After that, the mixture was filtered and concentrated under reduced pressure to give the residue, which was purified by flash chromatography (petroleum ether:ethyl acetate=1:1) to give tert-butyl N-[(3S)-1-{[3-methyl-5-(1-oxo-2,3-dihydroinden-5-yl)thiophen-2-yl]carbonyl}pyrrolidin-3-yl]carbamate (240 mg, 51.66% yield) as a yellow solid. LCMS (ESI) calc'd for C24H28N2O4S+ [M+H]+ m/z 441.18, found 441.2.

### Step 3 Synthesis of tert-butyl ((3S)-1-(5-(1-(dimethylamino)-2,3-dihydro-1H-inden-5-yl)-3-methylthiophene-2-carbonyl)pyrrolidin-3-yl)carbamate

To a solution of tert-butyl N-[(3S)-1-{[3-methyl-5-(1-oxo-2,3-dihydroinden-5-yl)thiophen-2-yl]carbonyl}pyrrolidin-3-yl]carbamate (200 mg, 0.454 mmol) in EtOH (10 mL) was added dimethylamine (2 N in MeOH, 2.6 mL) and 10 drops CH₃COOH, then the mixture was stirred at 60° C. for 2 h. NaBH₃CN (85.6 mg, 1.36 mmol) was added to the mixture, which was stirred at 60° C. for another 16 h. After that, the LCMS showed that the reaction was completed and the desired mass was detected. The resulting mixture was concentrated under reduced pressure to give the residue, which was purified by flash chromatography (petroleum ether:ethyl acetate=1:2) to afford tert-butyl ((3S)-1-(5-(1-(dimethylamino)-2,3-dihydro-1H-inden-5-yl)-3-methylthiophene-2-carbonyl)pyrrolidin-3-yl)carbamate (200 mg, 89% yield) as a yellow solid. LCMS (ESI) calc'd for C26H35N3O3S+ [M+H]+ m/z 470.24, found 470.2.

### Step 4 Synthesis of ((S)-3-aminopyrrolidin-1-yl)(5-(1-(dimethylamino)-2,3-dihydro-1H-inden-5-yl)-3-methylthiophen-2-yl)methanone

To tert-Butyl N-[(3S)-1-({5-[1-(dimethylamino)-2,3-dihydro-1H-inden-5-yl]-3-methylthiophen-2-yl}carbonyl)pyrrolidin-3-yl]carbamate (160 mg, 0.34 mmol) was added a solution of HCl (2M in EtOAc, 3 mL), then the mixture was stirred at rt for 6 h. The LCMS showed that the reaction was completed and the desired mass was detected. The mixture was concentrated under reduced pressure to give ((S)-3-aminopyrrolidin-1-yl)(5-(1-(dimethylamino)-2,3-dihydro-1H-inden-5-yl)-3-methylthiophen-2-yl)methanone (Compound 188, 95.5 mg, 74.35% yield) as a yellow solid. LCMS (ESI) calc'd for C21H27N3OS+ [M+H]+ m/z 370.13, found 370. ¹H NMR (400 MHz, MeOD) δ 7.60 (s, 1H), 7.58-7.53 (m, 2H), 7.25 (s, 1H), 4.93 (d, J=5.9 Hz, 1H), 3.96-3.85 (m, 2H), 3.80-3.70 (m, 1H), 3.70-3.55 (m, 2H), 3.17-3.07 (m, 1H), 3.04-2.92 (m, 1H), 2.78 (s, 3H), 2.61 (s, 3H), 2.52-2.32 (m, 3H), 2.28 (d, J=6.9 Hz, 3H), 2.06 (d, J=5.2 Hz, 1H).

The following compound was prepared analogously to Compound 188:

| Compound | Name | H NMR | MS |
|---|---|---|---|
| 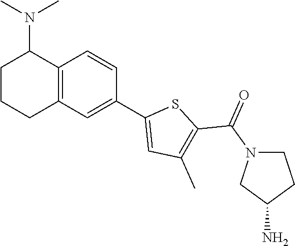 [SMILES: Cc1cc(-c2ccc3c(c2)CCCC3N(C)C)sc1C(=O)N1CC[C@H](N)C1] | ((S)-3- aminopyrrolidin- 1-yl)(5-(5- (dimethylamino)- 5,6,7,8- tetrahydronaphthalen- 2-yl)-3- methylthiophen- 2-yl)methanone | ¹H NMR (400 MHz, MeOD) δ 7.57- 7.48 (m, 2H), 7.45 (s, 1H), 7.23 (d, J = 6.8 Hz, 1H), 4.70 (t, J = 6.8 Hz, 1H), 3.96-3.86 (m, 2H), 3.80-3.56 (m, 3H), 2.89 (s, 3H), 2.85-2.70 (m, 2H), 2.60 (s, 3H), 2.36 (td, J = 14.1, 7.4 Hz, 1H), 2.27 (s, 3H), 2.23-2.12 (m, 1H), 2.11-1.94 (m, 3H), 1.78- 1.66 (m, 1H). | 384.1 [M + H]⁺ |
| 186 | | | |

### Example 59: Synthesis of (S)-(3-aminopyrrolidin-1-yl)(5-(4-(1-(dimethylamino)cyclobutyl)phenyl)-3-methylthiophen-2-yl)methanone (Compound 187)

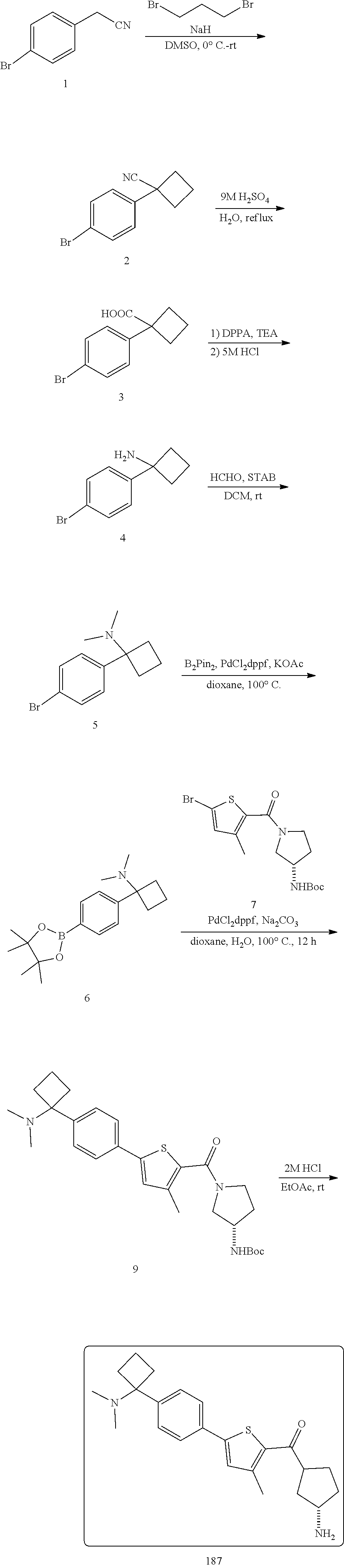

### Step 1 Synthesis of 1-(4-bromophenyl) cyclobutane-1-carbonitrile

To a solution of 2-(4-bromophenyl) acetonitrile (4 g, 20.4 mmol) in dry DMF (31 mL) was added NaH (2.08 g, 60% in oil, 51.0 mmol) in portions, and the solution was stirred for 1 h at 0° C. Then 1,3-dibromopropane (4.12 g, 20.4 mmol) was added to the above solution at 0° C. and the mixture was stirred at room temperature overnight. After completion of the reaction, the mixture was quenched by addition of water and extracted with ethyl acetate. The combined organic layer was washed with water twice and saturated NaCl solution once, dried over anhydrous Na₂SO₄, filtered and concentrated under reduced pressure to give the crude product, which was purified by silica gel column chromatography (petroleum ether:ethyl acetate=1:1) to afford 1-(4-bromophenyl) cyclobutane-1-carbonitrile (2.6 g, 51.47% yield) as a yellow oil.

LCMS (ESI) calc'd for C11H10BrN+ [M+H]+ m/z 236.00, found 236.0.

### Step 2 Synthesis of 1-(4-bromophenyl) cyclobutane-1-carboxylic acid

A solution of 1-(4-bromophenyl) cyclobutane-1-carbonitrile (2.6 g, 11.0 mmol) in 9 M H₂SO₄ (28 ml) was stirred at 100° C. overnight. After completion of the reaction, the mixture was diluted with water and then extracted with ethyl acetate. The combined organic layer was washed with water twice and saturated NaCl solution once and then dried over anhydrous Na₂SO₄ and evaporated in vacuum. The crude product was purified by silica gel column chromatography (petroleum ether:ethyl acetate=5:1) to give 1-(4-bromophenyl) cyclobutane-1-carboxylic acid (2.1 g, 70.91% yield) as a white solid. LCMS (ESI) calc'd for C11H10BrN+ [M+H]+ m/z 236.00, found 236.0.

### Step 3 Synthesis of 1-(4-bromophenyl) cyclobutan-1-amine

To a solution of 1-(4-bromophenyl) cyclobutane-1-carboxylic acid (2.1 g, 8.23 mmol) in toluene (28 mL) was added and TEA (1.83 g, 18.1 mmol) via a syringe under nitrogen, then DPPA (2.49 g, 9.05 mmol) was added and the mixture was stirred at 90° C. for 2 h. After completion of the reaction, the mixture was cooled to room temperature and diluted with EA. The combined organic layer was washed with NaHCO₃ twice and brine once, then dried over anhydrous Na₂SO₄ and evaporated in vacuum. To the mixture was added 5 M HCl solution (6 mL), then the resulting solution was stirred at 100° C. for 5 h. After cooling to room temperature, toluene was added to the mixture which was evaporated in vacuum. The obtained solid was dissolved into the saturated aq. NaHCO₃ (165 mL) and EtOAc (165 mL). The combined organic layer was washed with saturated aq. NaHCO₃ four times, dried over anhydrous Na₂SO₄ and evaporated in vacuum. The crude product was purified by silica gel column chromatography (petroleum ether:ethyl acetate=3:2, 2% triethylamine) to give the desired product (0.94 g, 47.56% yield) as a yellow solid. LCMS (ESI) calc'd for C10H12BrN+ [M+H]+ m/z 226.02, found 226.0.

### Step 4 Synthesis of 1-(4-bromophenyl)-N, N-dimethylcyclobutan-1-amine

To an eggplant type flask was added 1-(4-bromophenyl)cyclobutan-1-amine (940 mg, 4.16 mmol), DCM (54 mL), 37% HCHO (1.013 g, 12.47 mmol), Na₂SO₄ (175 mg, 2.50 mmol), and STAB (5.329 g, 25.0 mmol) successively and the mixture was stirred at room temperature for 2 h. Then the solution was concentrated under reduced pressure and ethyl acetate (35 mL) was added. The solution was washed with aq. NaHCO₃ (35 mL) and the aqueous layer was extracted with EtOAc (2*54 mL), dried over anhydrous Na₂SO₄ and evaporated in vacuum to give the crude product, which was purified by silica gel column chromatography (petroleum ether:ethyl acetate=3:2, 2% triethylamine) to give 1-(4-bromophenyl)-N, N-dimethylcyclobutan-1-amine (780 mg, 70.13% yield) as a yellow solid. LCMS (ESI) calc'd for C12H16BrN+ [M+H]+ m/z 254.05, found 254.0.

### Step 5 Synthesis of N,N-dimethyl-1-(4-(4,4,5,5-tetramethyl-1,3,2-dioxaborolan-2-yl)phenyl)cyclobutan-1-amine

To a solution of 1-(4-bromophenyl)-NN-dimethylcyclobutan-1-amine (300 mg, 1.18 mmol) in 1,4-dioxane (5 mL) was added B₂Pin₂ (329.70 mg, 1.30 mmol), Pd(dppf)Cl₂ (172.73 mg, 0.24 mmol) and potassium acetate (347.51 mg, 3.54 mmol), then the mixture was stirred under N₂ at 95° C. for 16 h. The LCMS showed that the reaction was completed and the desired mass was detected. After cooling to rt, the mixture was filtered and concentrated under reduced pressure to give N,N-dimethyl-1-(4-(4,4,5,5-tetramethyl-1,3,2-dioxaborolan-2-yl)phenyl)cyclobutan-1-amine (250 mg, 63.28% yield) as a yellow solid. LCMS (ESI) calc'd for C18H28BNO2+ [M+H]+ m/z 303.22, found 302.2.

### Step 6 Synthesis of tert-butyl (S)-(1-(5-(4-(1-(dimethylamino)cyclobutyl)phenyl)-3-methylthiophene-2-carbonyl)pyrrolidin-3-yl)carbamate

To a solution of N,N-dimethyl-1-[4-(4,4,5,5-tetramethyl-1,3,2-dioxaborolan-2-yl)phenyl]cyclobutan-1-amine (250 mg, 0.83 mmol) in dioxane/H₂O (v:v=8:1, 9 mL) was added tert-butyl N-((3S)-1-((5-bromo-3-methylthiophen-2-yl)carbonyl)pyrrolidin-3-yl)carbamate (323.09 mg, 0.83 mmol), Pd(dppf)Cl₂ (121.45 mg, 0.17 mmol) and K₃PO₄ (528.49 mg, 2.49 mmol), then the mixture was stirred under N₂ at 95° C. for 16 h. The LCMS showed that the reaction was completed and the desired mass was detected. After cooling to rt, the mixture was filtered and concentrated under reduced pressure to give the residue, which was purified by silica gel column chromatography (petroleum ether:ethyl acetate=1:1) to give the desired product (120 mg, 28.4% yield) as a yellow solid. LCMS (ESI) calc'd for C27H37N3O3S+ [M+H]+ m/z 484.26, found 484.2.

### Step 7 Synthesis of (S)-(3-aminopyrrolidin-1-yl)(5-(4-(1-(dimethylamino)cyclobutyl)phenyl)-3-methylthiophen-2-yl)methanone

To a solution of tert-butyl (S)-(1-(5-(4-(1-(dimethylamino)cyclobutyl)phenyl)-3-methylthiophene-2-carbonyl)pyrrolidin-3-yl)carbamate (120 mg, 0.25 mmol) in ethyl acetate (3 mL) was added HCl (2M in ethyl acetate, 5 mL), then the mixture was stirred at rt for 16 h. The LCMS showed that the reaction was completed and the desired mass was detected. The mixture was filtered and concentrated under reduced pressure to give (S)-(3-aminopyrrolidin-1-yl)(5-(4-(1-(dimethylamino)cyclobutyl)phenyl)-3-methylthiophen-2-yl)methanone (Compound 187, 60 mg, 59.90% yield) as a yellow solid. MS (ESI): mass calc'd for C22H29N3OS+ [M+H]+ m/z 384, found 384. ¹H NMR (400 MHz, MeOD) δ 7.84 (d, J=7.6 Hz, 2H), 7.75 (d, J=7.6 Hz, 2H), 7.40 (s, 1H), 4.02 (d, J=8.8 Hz, 2H), 3.90-3.68 (m, 3H), 2.97-2.74 (m, 4H), 2.59 (s, 6H), 2.47 (d, J=6.4 Hz, 1H), 2.38 (s, 3H), 2.18 (s, 1H), 2.01 (d, J=9.2 Hz, 1H), 1.65 (m, 1H).

### Example 60: Synthesis of (S)-(3-aminopyrrolidin-1-yl)(3-methyl-5-(4-(4-methylpiperidin-1-yl)phenyl)thiophen-2-yl)methanone (Compound 184)

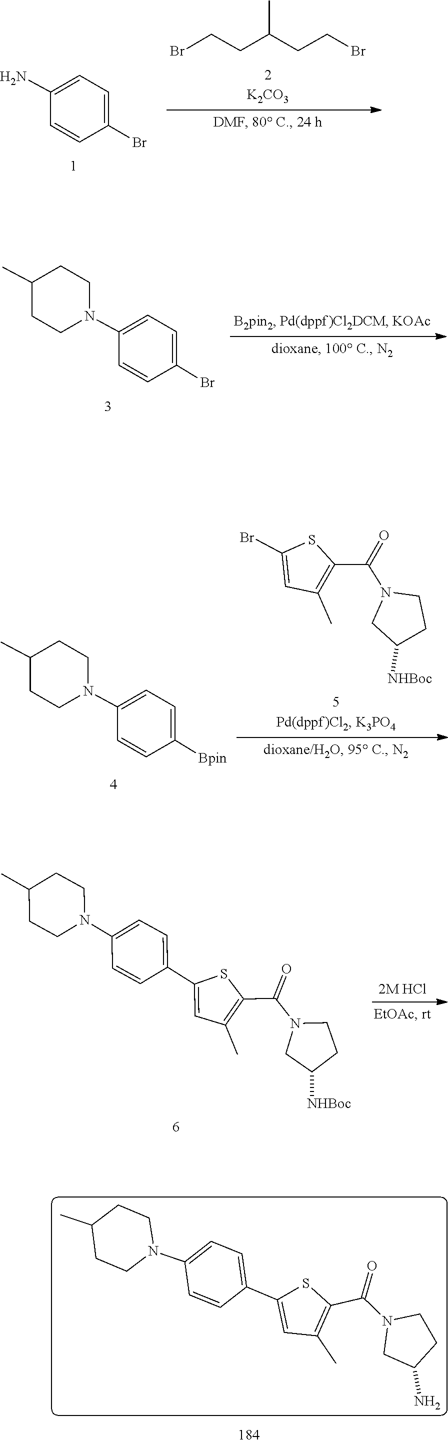

### Step 1 Synthesis of 1-(4-bromophenyl)-4-methylpiperidine

To a solution of 4-bromoaniline (1 g, 5.8 mmol) and K₂CO₃ (0.88 g, 6.3 mmol) in DMF (6 mL) was added 1,5-dibromo-3-methylpentane (1.56 g, 6.3 mmol) dropwise under N₂, then the reaction mixture was stirred at 80° C. for 24 h. The reaction was quenched with water and the mixture was extracted with ethyl acetate for three times. The combined organic phase was washed with brine, dried over Na₂SO₄, filtered and concentrated under reduced pressure to give the residue, which was purified by column chromatography (petroleum ether:ethyl acetate=5:1) to afford 1-(4-bromophenyl)-4-methylpiperidine (0.912 g, 62% yield) as an off-white solid.

¹H NMR (400 MHz, CDCl3) δ 7.30 (d, J=9.0 Hz, 2H), 6.79 (d, J=9.0 Hz, 2H), 3.59 (d, J=12.3 Hz, 2H), 2.67 (td, J=12.2, 2.3 Hz, 3H), 1.72 (d, J=13.3 Hz, 3H), 1.55-1.45 (m, 1H), 1.37-1.26 (m, 2H), 0.97 (d, J=6.5 Hz, 3H).

### Step 2 Synthesis of 4-methyl-1-(4-(4,4,5,5-tetramethyl-1,3,2-dioxaborolan-2-yl)phenyl)piperidine

To a solution of 1-(4-bromophenyl)-4-methylpiperidine (500 mg, 1.97 mmol) and 4,4,5,5-tetramethyl-2-(4,4,5,5-tetramethyl-1,3,2-dioxaborolan-2-yl)-1,3,2-dioxaborolane (600 mg, 2.36 mmol) in 1,4-dioxane (5 mL) was added KOAc (579 mg, 5.9 mmol) and Pd(dppf)Cl₂·DCM (341 mg, 0.295 mmol), then the reaction mixture was stirred at 100° C. under N₂ for 16 h. After the reaction was completed, the solvent was removed under reduced pressure to give the residue which was purified by column chromatography (petroleum ether:ethyl acetate=1:1) to afford 4-methyl-1-(4-(4,4,5,5-tetramethyl-1,3,2-dioxaborolan-2-yl)phenyl)piperidine (567 mg, 90.9% yield) as a yellow solid. MS (ESI): mass calc'd. for C18H29BNO2+ [M+1]+ m/z 302.23, found 302.

### Step 3 Synthesis of tert-butyl (S)-(1-(3-methyl-5-(4-(1-methylpiperidin-4-yl)phenyl)thiophene-2-carbonyl)pyrrolidin-3-yl)carbamate

To a solution of 4-methyl-1-(4-(4,4,5,5-tetramethyl-1,3,2-dioxaborolan-2-yl)phenyl)piperidine (139 mg, 0.4623 mmol) and tert-butyl (S)-(1-(5-bromo-3-methylthiophene-2-carbonyl)pyrrolidin-3-yl)carbamate (150 mg, 0.3853 mmol) in 1,4-dioxane/H₂O (v/v=8:1, 9 mL) was added K₃PO₄ (245.36 mg, 1.156 mmol) and Pd(dppf)Cl₂·DCM (63 mg, 0.077 mmol), then the reaction mixture was stirred at 95° C. under N₂ for 16 h. After the reaction was completed, the solvent was removed under reduced pressure to give the residue, which was purified by column chromatography (petroleum ether:ethyl acetate=1:1) to afford tert-butyl (S)-(1-(3-methyl-5-(4-(1-methylpiperidin-4-yl)phenyl)thiophene-2-carbonyl)pyrrolidin-3-yl)carbamate (172 mg, 83.1%) as a yellowish solid. MS (ESI): mass calc'd. for C27H37N3O3S+ [M+1]+ m/z 484.26, found 484.2.

### Step 4 Synthesis of (S)-(3-aminopyrrolidin-1-yl)(3-methyl-5-(4-(4-methylpiperidin-1-yl)phenyl)thiophen-2-yl)methanone

To a solution of tert-butyl (S)-(1-(3-methyl-5-(4-(1-methylpiperidin-4-yl)phenyl)thiophene-2-carbonyl)pyrrolidin-3-yl)carbamate (110 mg, 0.2274 mmol) in EtOAc (2 mL) was added HCl (2M in EtOAc, 4 mL), then the reaction mixture was stirred at 25° C. for 12 h. After the reaction was completed, the solvent was removed under reduced pressure to give the residue, which was purified by prep-HPLC to afford (S)-(3-aminopyrrolidin-1-yl)(3-methyl-5-(4-(1-methylpiperidin-4-yl)phenyl)thiophen-2-yl)methanone (Compound 184, 53.5 mg, 58.9%) as a yellow solid. MS (ESI): mass calc'd. for C22H30N3OS+ [M+1]+384.21, found 384. ¹H NMR (400 MHz, MeOD) δ 7.85 (q, J=8.9 Hz, 4H), 7.39 (s, 1H), 4.03-3.98 (m, 2H), 3.89-3.82 (m, 1H), 3.80-3.66 (m, 6H), 2.51-2.42 (m, 1H), 2.37 (s, 3H), 2.20-2.14 (m, 1H), 2.06 (d, J=13.3 Hz, 2H), 1.99-1.79 (m, 3H), 1.10 (d, J=5.9 Hz, 3H).

The following compounds are prepared analogously to the methods described in this example:

| Compound | Synthesis |
|---|---|
| 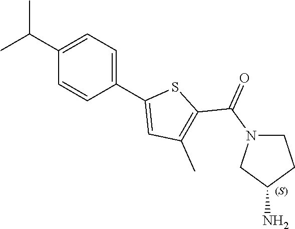 | For step 1, a corresponding methylating reagent can be used to synthesize compound 214 |
| 214 | |
| 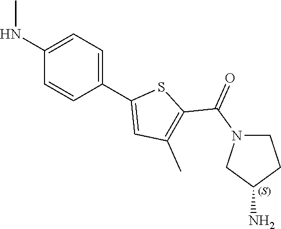 | For step 1, a corresponding methylating reagent can be used to synthesize compound 215; an additional protecting step may be used |
| 215 | |

### Example 61: Synthesis of (S)-(3-aminopyrrolidin-1-yl)(5-(4-(3-(dimethylamino)oxetan-3-yl)phenyl)-3-methylthiophen-2-yl)methanone (Compound 221)

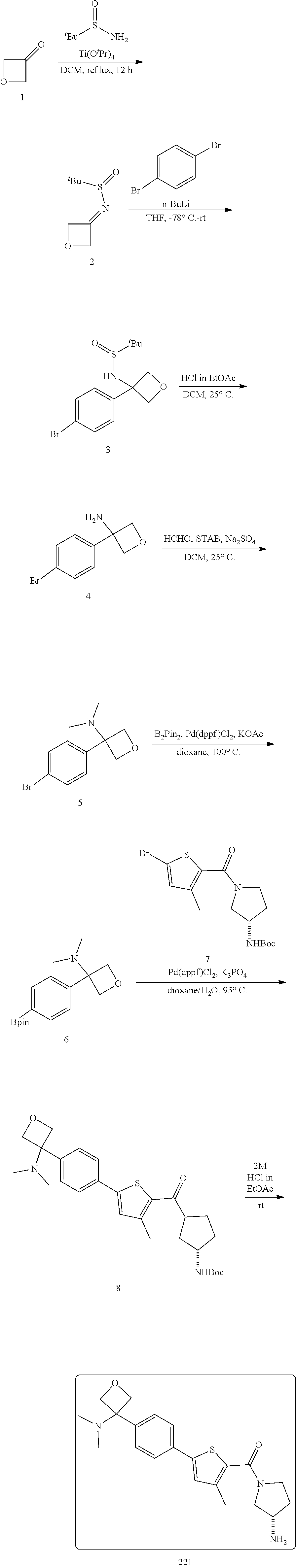

### Step 1 Synthesis of 2-methyl-N-(oxetan-3-ylidene)propane-2-sulfinamide

To a solution of oxetan-3-one (1.00 g, 13.90 mmol) and 2-methylpropane-2-sulfinamide (2.02 g, 16.60 mmol) in DCM (15 mL) was added titanium tetraisopropanolate (7.90 g, 27.80 mmol) at 25° C., then the mixture was stirred at 45° C. for 16 hours. LCMS showed that the reaction was completed and the desired mass was detected. The reaction mixture was poured into saturated aq. NaHCO₃ (50 mL) and stirred vigorously for 20 minutes, then the suspension was filtered through a pad of celite. The filter cake was washed with dichloromethane, and the filtrate was extracted with dichloromethane, washed with brine, dried over Na₂SO₄, filtered and concentrated under reduced pressure to give the crude product, which was purified by column chromatography on silica gel (petroleum ether:ethyl acetate=3:1) to afford 2-methyl-N-(oxetan-3-ylidene)propane-2-sulfinamide (1.50 g, 90.0% yield) as yellow oil.

### Step 2 Synthesis of N-(3-(4-bromophenyl)oxetan-3-yl)-2-methylpropane-2-sulfinamide

To a mixture of 2-methyl-N-(oxetan-3-ylidene)propane-2-sulfinamide (1.62 g, 6.85 mmol) in THF (20 mL) was added n-BuLi (2.4 M, 371 mg) dropwise at −78° C. for 30 mins, then the mixture was stirred at −78° C. for 1 hour. After that, a solution of 2-methyl-N-(oxetan-3-ylidene)propane-2-sulfinamide (800 mg, 4.56 mmol) in THF (4 mL) was added dropwise for 30 mins, and the mixture was stirred at −78° C. for 30 mins before it was allowed to warm to rt and stirred for additional 1 hour. LCMS showed the desired mass was detected. The reaction mixture was quenched by saturated aq. NH₄C₁ (50 mL) and extracted with EtOAc (25 mL×3), and the combined organic layer was washed with saturated aq. NaHCO₃ (100 mL) and brine (100 mL), dried over Na₂SO₄, filtered and concentrated under reduced pressure to give the crude product, which was purified by column chromatography on silica gel (ethyl acetate:petroleum ether=28%) to afford N-(3-(4-bromophenyl)oxetan-3-yl)-2-methylpropane-2-sulfinamide (200 mg, 85% yield) as light yellow oil.

### Step 3 Synthesis of 3-(4-bromophenyl)oxetan-3-amine

To a mixture of N-(3-(4-bromophenyl)oxetan-3-yl)-2-methylpropane-2-sulfinamide (320 mg, 0.96 mmol) in DCM (19 mL) was added HCl (2 M in ethyl acetate, 0.96 mL) dropwise at 25° C., then it was stirred at 25° C. for 0.5 hour. LCMS showed the reaction was completed and the desired mass was detected. The reaction mixture was diluted with petroleum ether and filtered, then the filtrate was concentrated under reduced pressure to give the crude product (228 mg, 90.0% yield) as a white solid, which was used to the next step without further purification.

MS (ESI): mass calc'd. for C9H10BrNO+ [M+H]+ m/z 227.99, found 228.0.

### Step 4 Synthesis of 3-(4-bromophenyl)-NN-dimethyloxetan-3-amine

To a solution of 3-(4-bromophenyl)oxetan-3-amine (228 mg, 1.00 mmol) in DCM (20 mL) was added STAB (1.27 g, 6.00 mmol), aqueous formaldehyde solution (90 mg, 3.00 mmol) and Na₂SO₄ (43 mg, 0.30 mmol) successively, then it was stirred at 25° C. for 6 hours. LCMS showed the reaction was completed and the desired mass was detected. The reaction mixture was concentrated under reduced pressure to give the residue, which was diluted with H₂O (50 mL) and extracted with EtOAc (30 mL×3). The combined organic layers were washed with brine (100 mL×2), dried over Na₂SO₄, filtered and concentrated under reduced pressure to give the crude product, which was purified by column chromatography on silica gel (ethyl acetate:petroleum ether=0%~100%) to afford 3-(4-bromophenyl)-N,N-dimethyloxetan-3-amine (178 mg, 59.09% yield) as yellow oil. MS (ESI): mass calc'd. for C11H14BrNO+ [M+H]+ m/z 256.03, found 256.0.

### Step 5 Synthesis of N,N-dimethyl-3-(4-(4,4,5,5-tetramethyl-1,3,2-dioxaborolan-2-yl)phenyl)oxetan-3-amine

To a solution of 3-(4-bromophenyl)-NN-dimethyloxetan-3-amine (178 mg, 0.69 mmol) in 1,4-dioxane (6 mL) was added B₂Pin₂ (194 mg, 0.76 mmol), potassium acetate (205 mg, 2.08 mmol) and Pd(dppf)Cl₂ (36 mg, 0.05 mmol) under nitrogen, then the mixture was stirred at 100° C. for 12 hours under nitrogen atmosphere. LCMS showed the reaction was completed and the desired mass was detected. The reaction mixture was concentrated under reduced pressure to give the crude product, which was purified by column chromatography on silica gel (ethyl acetate:petroleum ether=0%~100%) to give N,N-dimethyl-3-(4-(4,4,5,5-tetramethyl-1,3,2-dioxaborolan-2-yl)phenyl)oxetan-3-amine (126 mg, 47.8% yield) as a yellow solid.

MS (ESI): mass calc'd. for C17H26BNO3+ [M+H]+ m/z 304.20, found 304.2.

### Step 6 Synthesis of tert-butyl (S)-(1-(5-(4-(3-(dimethylamino)oxetan-3-yl)phenyl)-3-methylthiophene-2-carbonyl)pyrrolidin-3-yl)carbamate

To a mixture of N,N-dimethyl-3-(4-(4,4,5,5-tetramethyl-1,3,2-dioxaborolan-2-yl)phenyl)oxetan-3-amine (126 mg, 0.66 mmol) in 1,4-dioxane (8 mL) and H₂O (1 mL) was added tert-butyl N-[(3S)-1-[(5-bromo-3-methylthiophen-2-yl)carbonyl]pyrrolidin-3-yl]carbamate (308 mg, 0.79 mmol), K₃PO₄ (420 mg, 1.98 mmol) and Pd(dppf)Cl₂·DCM (108 mg, 0.13 mmol). Then the mixture was stirred at 95° C. for 12 hours under N₂ atmosphere. LCMS showed the reaction was completed and the desired mass was detected. The reaction mixture was concentrated under reduced pressure to give the crude product, which was purified by column chromatography on silica gel (ethyl acetate:petroleum ether=1:1) to afford tert-butyl (S)-(1-(5-(4-(3-(dimethylamino)oxetan-3-yl)phenyl)-3-methylthiophene-2-carbonyl)pyrrolidin-3-yl)carbamate (170 mg, 42.5% yield) as yellow oil. MS (ESI): mass calc'd. for C26H35N3O4S+ [M+H]+ m/z 486.23, found 486.2.

### Step 7 Synthesis of (S)-(3-aminopyrrolidin-1-yl)(5-(4-(3-(dimethylamino)oxetan-3-yl)phenyl)-3-methylthiophen-2-yl)methanone

To a solution of tert-butyl (S)-(1-(5-(4-(3-(dimethylamino)oxetan-3-yl)phenyl)-3-methylthiophene-2-carbonyl)pyrrolidin-3-yl)carbamate (300 mg, 0.62 mmol) in EtOAc (3 mL) was added HCl (2 M in EtOAc, 3 mL), then the mixture was stirred at 25° C. for 12 hours. LCMS showed that the desired mass was detected. The reaction mixture was concentrated under reduced pressure to give the crude product, which was purified by prep-HPLC to afford (S)-(3-aminopyrrolidin-1-yl)(5-(4-(3-(dimethylamino)oxetan-3-yl)phenyl)-3-methylthiophen-2-yl)methanone (Compound 221, 11.6 mg, 4.7% yield) as a white solid. MS (ESI): mass calc'd. for C21H27N3O2S+ [M+H]+ m/z 386.18, found 386. ¹H NMR (400 MHz, MeOD) δ 7.69 (d, J=8.4 Hz, 2H), 7.28 (s, 1H), 7.23 (d, J=8.4 Hz, 2H), 4.71-4.48 (m, 5H), 3.81-3.73 (m, 2H), 3.67-3.55 (m, 2H), 2.34 (s, 3H), 2.22-2.13 (m, 1H), 2.06 (s, 6H), 1.86-1.77 (m, 1H).

### Example 62: Synthesis of ((S)-3-aminopyrrolidin-1-yl)(3-methyl-5-(4-(1-(1-methylpyrrolidin-3-yl)piperidin-4-yl)phenyl)thiophen-2-yl)methanone (Compound 170)

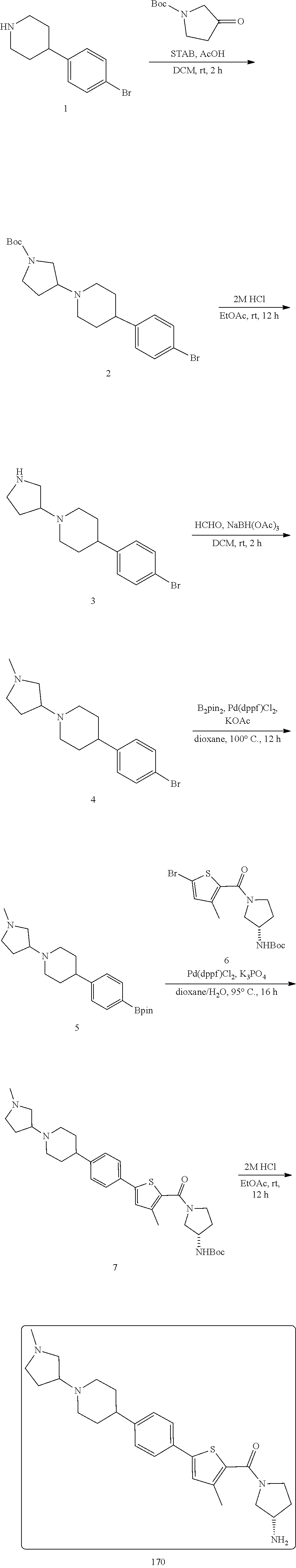

### Step 1 Synthesis of tert-butyl 3-(4-(4-bromophenyl)piperidin-1-yl)pyrrolidine-1-carboxylate

To a mixture of tert-butyl (3-oxopyrrolidin-1-yl) formate (1.00 g, 5.40 mmol) in DCM (40 mL) was added 4-(4-bromophenyl)piperidine (1.30 g, 5.40 mmol), STAB (1.60 g, 7.50 mmol) and AcOH (0.32 g, 5.40 mmol), then the mixture was stirred at 25° C. for 2 hours. LCMS showed the reaction was completed and the desired mass was detected. The reaction was quenched by addition of NaOH (1 M, 15 mL), then the mixture was concentrated to dryness, diluted with EtOAc (30 mL), washed with H₂O (50 mL) and brine (50 mL). The combined organic layer was concentrated under reduced pressure to give the crude product, which was purified by column chromatography on silica gel (ethyl acetate:petroleum ether=20:80) to afford tert-butyl 3-(4-(4-bromophenyl)piperidin-1-yl)pyrrolidine-1-carboxylate (1.60 g, 68.5% yield) as a white solid. MS (ESI): mass calc'd. for C20H29BrN2O2+ [M+H]+ m/z 409.14, found 409.1.

### Step 2 Synthesis of 4-(4-bromophenyl)-1-(pyrrolidin-3-yl)piperidine

To a solution of tert-butyl 3-(4-(4-bromophenyl)piperidin-1-yl)pyrrolidine-1-carboxylate (600 mg, 1.46 mmol) in EtOAc (4 mL) was added HCl (2 M in EtOAc, 6 mL) dropwise, then the mixture was stirred at 25° C. for 12 hours. LCMS showed the desired mass was observed. After that, NaOH (1 M, 12 mL) was added to adjust pH to 8-9, then the solution was concentrated under reduced pressure to give the crude product (470 mg, 94% yield) as light-yellow oil, which was used for the next step without further purification. MS (ESI): mass calc'd. for C15H21BrN2+ [M+H]+ m/z 309.09, found 309.1.

### Step 3 Synthesis of 4-(4-bromophenyl)-1-(1-methylpyrrolidin-3-yl)piperidine

To a solution of 4-(4-bromophenyl)-1-(pyrrolidin-3-yl)piperidine (470 mg, 1.52 mmol) in DCM (50 mL) was added STAB (1.93 g, 9.12 mmol), HCHO (137 mg, 4.56 mmol) and Na₂SO₄ (65 mg, 0.46 mmol), then it was stirred at 25° C. for 12 hours. LCMS showed the reaction was completed and the desired mass was detected. The reaction mixture was concentrated to dryness, which was diluted with saturated aq. NaHCO₃ (50 mL) and extracted with EtOAc (30 mL×3). The combined organic layer was washed with brine (100 mL), dried over Na₂SO₄, filtered and concentrated under reduced pressure to give the crude product, which was purified by column chromatography on silica gel (ethyl acetate:petroleum ether=0%~100%) to afford 4-(4-bromophenyl)-1-(1-methylpyrrolidin-3-yl)piperidine (424 mg, 77.6% yield) as yellow oil.

MS (ESI): mass calc'd. for C16H23BrN2+ [M+H]+ m/z 323.10, found 323.1.

### Step 4 Synthesis of 1-(1-methylpyrrolidin-3-yl)-4-(4-(4,4,5,5-tetramethyl-1,3,2-dioxaborolan-2-yl)phenyl)piperazine

To a mixture of 4-(4-bromophenyl)-1-(1-methylpyrrolidin-3-yl)piperidine (424 mg, 1.31 mmol) in 1,4-dioxane (11 mL) was added B₂Pin₂ (366 mg, 1.44 mmol), Pd(dppf)Cl₂·DCM (214 mg, 0.26 mmol) and potassium acetate (386 mg, 3.93 mmol), then the mixture was stirred at 100° C. for 12 hours under nitrogen atmosphere. LCMS showed the reaction was completed and the desired mass was detected. The reaction mixture was concentrated under reduced pressure to give the crude product, which was purified by column chromatography on silica gel (petroleum ether:ethyl acetate=1:1) to afford 1-(1-methylpyrrolidin-3-yl)-4-(4-(4,4,5,5-tetramethyl-1,3,2-dioxaborolan-2-yl)phenyl)piperazine (320 mg, 52.7% yield) as a yellow solid.

MS (ESI): mass calc'd. for C21H34BN3O2+ [M+H]+ m/z 372.27, found 372.2.

### Step 5 Synthesis of tert-butyl ((3S)-1-(3-methyl-5-(4-(1-(1-methylpyrrolidin-3-yl)piperidin-4-yl)phenyl)thiophene-2-carbonyl)pyrrolidin-3-yl)carbamate

To a solution of 1-(1-methylpyrrolidin-3-yl)-4-(4-(4,4,5,5-tetramethyl-1,3,2-dioxaborolan-2-yl)phenyl)piperazine (320 mg, 0.86 mmol) and tert-butyl (S)-(1-(5-bromo-3-methylthiophene-2-carbonyl)pyrrolidin-3-yl)carbamate (269 mg, 0.69 mmol) in 1,4-dioxane (8 mL) and H₂O (1.5 mL) was added K₃PO₄ (550 mg, 2.59 mmol) and Pd(dppf)Cl₂ (141 mg, 0.17 mmol), then the mixture was stirred at 95° C. for 16 hours under nitrogen atmosphere. LCMS showed the reaction was completed and the desired mass was detected. The reaction mixture was concentrated under reduced pressure to give the crude product, which was purified by column chromatography on silica gel (petroleum ether:ethyl acetate=1:1) to afford tert-butyl ((3S)-1-(3-methyl-5-(4-(1-(1-methylpyrrolidin-3-yl)piperidin-4-yl)phenyl)thiophene-2-carbonyl)pyrrolidin-3-yl)carbamate (600 mg, crude) as yellow oil. MS (ESI): mass calc'd. for C31H44N4O3S+ [M+H]+ m/z 553.31, found 553.2.

### Step 6 Synthesis of ((S)-3-aminopyrrolidin-1-yl)(3-methyl-5-(4-(1-(1-methylpyrrolidin-3-yl)piperidin-4-yl)phenyl)thiophen-2-yl)methanone

To a solution of tert-butyl ((3S)-1-(3-methyl-5-(4-(1-(1-methylpyrrolidin-3-yl)piperidin-4-yl)phenyl)thiophene-2-carbonyl)pyrrolidin-3-yl)carbamate (50 mg, 0.09 mmol) in EtOAc (1 mL) was added HCl (2 M in EtOAc, 2 mL) dropwise, then the mixture was stirred at 25° C. for 12 hours. LCMS showed the reaction was completed and the desired mass was detected. The reaction mixture was concentrated under reduced pressure under reduced pressure to give the crude product, which was purified by prep-HPLC to afford ((S)-3-aminopyrrolidin-1-yl)(3-methyl-5-(4-(1-(1-methylpyrrolidin-3-yl)piperidin-4-yl)phenyl)thiophen-2-yl)methanone (Compound 170, 11.8 mg, 27.6% yield) as a white solid. MS (ESI): mass calc'd. for C26H36N4OS+ [M+H]+ m/z 452.26, found 453.3. ¹H NMR (400 MHz, MeOD) δ 7.62 (d, J=8.0 Hz, 2H), 7.33 (d, J=8.0 Hz, 2H), 7.25 (s, 1H), 4.15 (s, 1H), 4.02-3.96 (m, 2H), 3.94-3.55 (m, 8H), 3.45 (s, 1H), 3.23-3.10 (m, 2H), 3.02 (s, 3H), 2.99-2.89 (m, 1H), 2.64 (s, 1H), 2.51-2.39 (m, 2H), 2.36 (s, 3H), 2.18-2.05 (m, 5H).

### Example 63: Synthesis of 2-methoxyethyl (S)-(4-(5-(3-aminopyrrolidine-1-carbonyl)-4-methylthiophen-2-yl)benzyl)(methyl)carbamate (Compound 226)

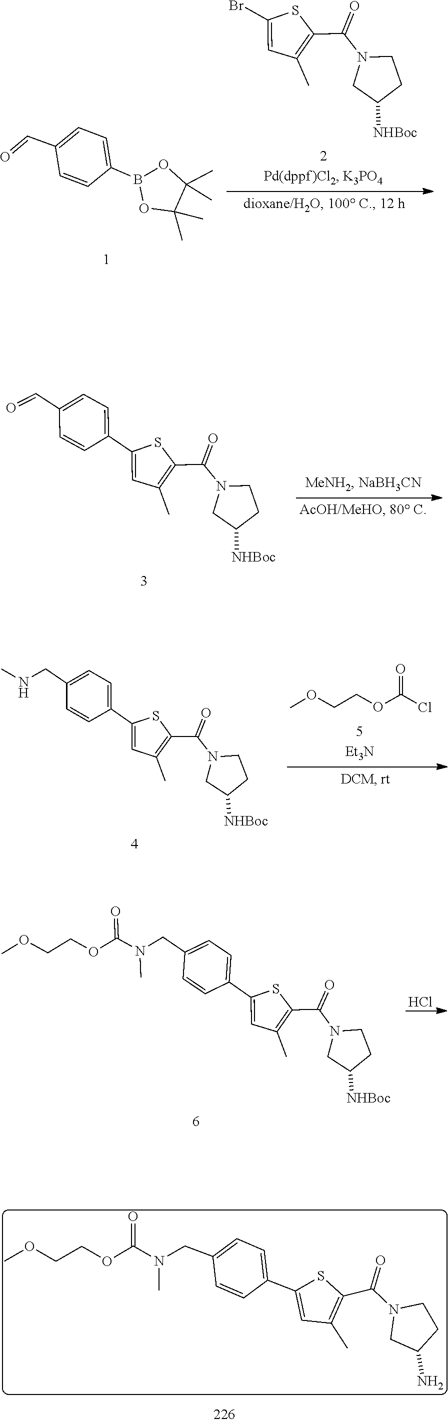

### Step 1 Synthesis of tert-butyl (S)-(1-(5-(4-formylphenyl)-3-methylthiophene-2-carbonyl)pyrrolidin-3-yl)carbamate

A mixture of tert-butyl (S)-(1-(5-bromo-3-methylthiophene-2-carbonyl)pyrrolidin-3-yl)carbamate (2 g, 0.0051 mol), 4-(4,4,5,5-tetramethyl-1,3,2-dioxaborolan-2-yl)benzaldehyde (1.3 g, 5.6 mmol), Pd(dppf)Cl₂·DCM (0.42 g, 5.0 mmol), K₃PO₄ (2.17 g, 10.2 mmol) in 1,4-dioxane/H₂O (v:v=10:1, 22 mL) was stirred at 95° C. for 16 h under nitrogen. After the completion, the mixture was diluted with H₂O and the resulting mixture was extracted with EtOAc for 3 times. The combined organic phase was washed with brine, dried over Na₂SO₄, filtered and concentrated under reduced pressure to give the residue, which was purified by flash column chromatography using petroleum ether/EtOAc=1:1 as eluent to afford tert-butyl (S)-(1-(5-(4-formylphenyl)-3-methylthiophene-2-carbonyl)pyrrolidin-3-yl)carbamate (800 mg, 37.25% yield) as yellow oil. LCMS (ESI) calculated for C22H26N2O4S+ [M+H]+ m/z 415.16, found 415.05.

### Step 2 Synthesis of tert-butyl (S)-(1-(3-methyl-5-(4-((methylamino)methyl)phenyl)thiophene-2-carbonyl)pyrrolidin-3-yl)carbamate

To a solution of tert-butyl (S)-(1-(5-(4-formylphenyl)-3-methylthiophene-2-carbonyl)pyrrolidin-3-yl)carbamate (800 mg, 1.93 mmol) in MeOH (10 mL) was added MeNH₂·HCl (1.30 g, 19.3 mmol), NaBH₃CN (182 mg, 2.895 mmol), and triethylamine (2.93 g, 28.95 mmol) then the mixture was stirred at rt for 16 h. The resulting mixture was concentrated under reduced pressure to give the residue, which was purified by flash chromatography to afford tert-butyl (S)-(1-(3-methyl-5-(4-((methylamino)methyl)phenyl)thiophene-2-carbonyl)pyrrolidin-3-yl)carbamate (800 mg, 96.49% yield) as a yellow solid. LCMS (ESI) calc'd for C23H31N3O3S+ [M+H]+ m/z 430.21, found 430.25.

### Step 3 Synthesis of 2-methoxyethyl (S)-(4-(5-(3-((tert-butoxycarbonyl)amino)pyrrolidine-1-carbonyl)-4-methylthiophen-2-yl)benzyl)(methyl)carbamate

To a solution of tert-butyl (S)-(1-(3-methyl-5-(4-((methylamino)methyl)phenyl)thiophene-2-carbonyl)pyrrolidin-3-yl)carbamate (300 mg, 0.6984 mmol) and TEA (212 mg, 2.0952 mmol) in DCM (5 mL) was added chloro(2-methoxyethoxy)methanone (145 mg, 1.0476 mmol), then the mixture was stirred at 25° C. for 16 h. The resulting mixture was concentrated under reduced pressure to give the residue, which was purified by flash chromatography to afford 2-methoxyethyl (S)-(4-(5-(3-((tert-butoxycarbonyl)amino)pyrrolidine-1-carbonyl)-4-methylthiophen-2-yl)benzyl)(methyl)carbamate (316 mg, 85.11% yield) as a white solid. LCMS (ESI) calculated for C27H37N3O6S+ [M+H]+ m/z 532.24, found 532.20.

### Step 4 Synthesis of 2-methoxyethyl (S)-(4-(5-(3-aminopyrrolidine-1-carbonyl)-4-methylthiophen-2-yl)benzyl)(methyl)carbamate

To a solution of 2-methoxyethyl (S)-(4-(5-(3-((tert-butoxycarbonyl)amino)pyrrolidine-1-carbonyl)-4-methylthiophen-2-yl)benzyl)(methyl)carbamate (300 mg, 0.5643 mmol) in EtOAc (2 mL) was added HCl (4 M in 1,4-dioxane, 5 mL), then the mixture was stirred at 25° C. for 16 hrs. After the completion, the solvent was removed under reduced pressure to give the residue, which was purified by prep-HPLC to afford 2-methoxyethyl (S)-(4-(5-(3-aminopyrrolidine-1-carbonyl)-4-methylthiophen-2-yl)benzyl)(methyl)carbamate (Compound 226, 86 mg, 35.32% yield) as a yellowish solid. LCMS (ESI) calculated for C22H29N3O4S+ [M+H]+ m/z 432.19, found 432.10. ¹H NMR (400 MHz, MeOD) δ 7.62 (d, J=8.2 Hz, 2H), 7.31 (d, J=8.0 Hz, 2H), 7.26 (s, 1H), 4.51 (s, 2H), 4.26 (dd, J=5.4, 3.8 Hz, 2H), 3.99 (t, J=7.3 Hz, 2H), 3.88-3.75 (m, 1H), 3.74-3.61 (m, 2H), 3.37 (d, J=12.2 Hz, 3H), 2.89 (s, 3H), 2.49-2.36 (m, 1H), 2.36 (s, 3H), 2.16-2.10 (m, 1H).

### Example 64: Synthesis of (S)-(3-aminopyrrolidin-1-yl)(3-methyl-5-(3-methyl-4-(tetrahydro-2H-pyran-4-yl)phenyl)thiophen-2-yl)methanone (Compound 161)

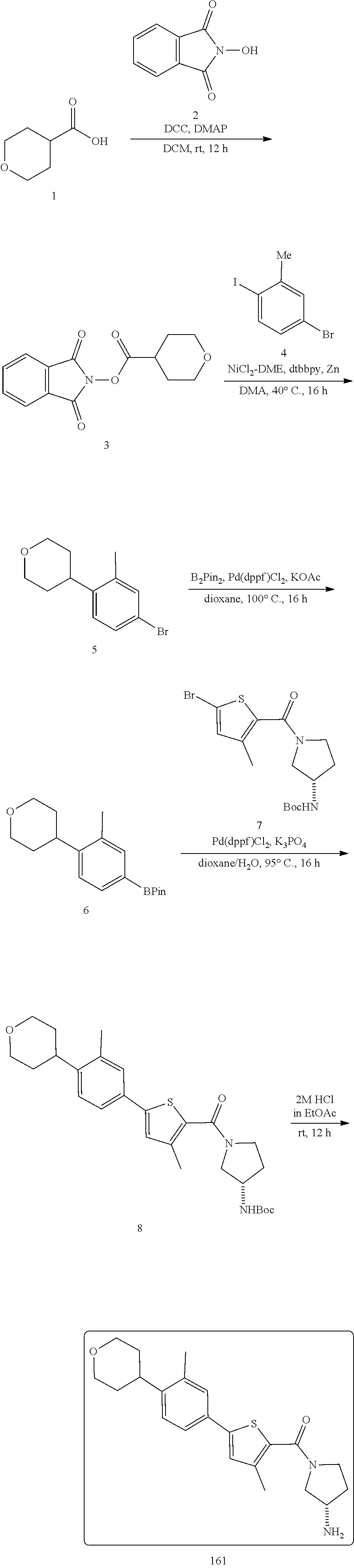

### Step 1 Synthesis of 1,3-dioxoisoindolin-2-yl tetrahydro-2H-pyran-4-carboxylate

To a solution of oxane-4-carboxylic acid (10 g, 76.8 mmol), 2-hydroxyisoindole-1,3-dione (12.53 g, 76.8 mmol) and DMAP (0.47 g, 3.8 mmol) in DCM (80 mL) was added a solution of DCC (17.43 g, 84.4 mmol) in DCM (20 mL), then the mixture was stirred at 25° C. for 12 h. After the reaction was completed, the solvent was removed under reduced pressure to give the residue, which was purified by column chromatography (dichloromethane:methanol=10:1) to afford 1,3-dioxoisoindol-2-yl oxane-4-carboxylate (18.9 g, 87.6% yield) as a yellowish solid. ¹H NMR (400 MHz, CDCl3) δ 7.91-7.87 (m, 2H), 7.82-7.78 (m, 2H), 4.02 (dt, J=11.8, 3.8 Hz, 2H), 3.54-3.51 (m, 2H), 3.04-2.97 (m, 1H), 2.06-1.94 (m, 4H).

### Step 2 Synthesis of 4-(4-bromo-2-methylphenyl)tetrahydro-2H-pyran

To a solution of 4-bromo-1-iodo-2-methylbenzene (600 mg, 2.0207 mmol) and 1,3-dioxoisoindol-2-yl oxane-4-carboxylate (834.3 mg, 3.03 mmol) in DMA (9 mL) was added NiCl₂·DME (88.9 mg, 0.40 mmol), dtbbpy (119.3 mg, 0.44 mmol) and Zn powder (264.3 mg, 4.04 mmol), then the mixture was stirred at 45° C. under nitrogen for 16 hours. After the reaction was completed, the mixture was diluted with H₂O and extracted with EtOAc for 3 times. The combined organic phase was washed with brine, dried over Na₂SO₄, filtered and concentrated under reduced pressure to give the residue, which was purified by column chromatography (petroleum ether:ethyl acetate=1:1) to afford 4-(4-bromo-2-methylphenyl)oxane (270 mg, 51.8% yield) as a white solid. ¹H NMR (400 MHz, CDCl3) δ 7.32-7.30 (m, 2H), 7.08 (d, J=8.0 Hz, 1H), 4.11-4.07 (m, 2H), 3.54 (td, J=11.8, 2.0 Hz, 2H), 2.95-2.87 (m, 1H), 2.32 (s, 3H), 1.83-1.73 (m, 2H), 1.67-1.63 (m, 2H).

### Step 3 Synthesis of 4,4,5,5-tetramethyl-2-(3-methyl-4-(tetrahydro-2H-pyran-4-yl)phenyl)-1,3,2-dioxaborolane

To a solution of 4-(4-bromo-2-methylphenyl)oxane (270 mg, 1.0582 mmol), B₂Pin₂ (323 mg, 1.2698 mmol) and KOAc (312 mg, 3.1746 mmol) in dioxane (6 mL) was added Pd(dppf)Cl₂·DCM (173 mg, 0.2116 mmol), then the mixture was stirred at 95° C. under nitrogen for 16 hours. After the reaction was completed, the solvent was removed under reduced pressure to give the residue, which was purified by column chromatography (petroleum ether:ethyl acetate=1:1) to afford 4,4,5,5-tetramethyl-2-(3-methyl-4-(tetrahydro-2H-pyran-4-yl)phenyl)-1,3,2-dioxaborolane (160 mg, yield=49.53%) as a white solid. ¹H NMR (400 MHz, CDCl3) δ 7.65 (d, J=7.8 Hz, 1H), 7.62 (s, 1H), 7.25 (d, J=7.8 Hz, 1H), 4.09 (dd, J=11.4, 4.2 Hz, 2H), 3.55 (td, J=11.8, 2.0 Hz, 2H), 3.03-2.95 (m, 1H), 2.36 (s, 3H), 1.89-1.78 (m, 2H), 1.69-1.65 (m, 2H), 1.33 (s, 12H).

### Step 4 Synthesis of tert-butyl (S)-(1-(3-methyl-5-(3-methyl-4-(tetrahydro-2H-pyran-4-yl)phenyl)thiophene-2-carbonyl)pyrrolidin-3-yl)carbamate

To a solution of tert-butyl (S)-(1-(5-bromo-3-methylthiophene-2-carbonyl)pyrrolidin-3-yl)carbamate (170 mg, 0.4367 mmol), 4,4,5,5-tetramethyl-2-(3-methyl-4-(tetrahydro-2H-pyran-4-yl)phenyl)-1,3,2-dioxaborolane (145 mg, 0.4803 mmol) and K₃PO₄ (278 mg, 1.3101 mmol) in dioxane/H₂O (9 ml, v:v=8:1) was added Pd(dppf)Cl₂·DCM (71.3 mg, 0.0873 mmol), then the mixture was stirred at 95° C. under nitrogen for 16 hours. After the reaction was completed, the solvent was removed under reduced pressure to give the residue, which was purified by column chromatography (dichloromethane:methanol=10:1) to afford tert-butyl (S)-(1-(3-methyl-5-(3-methyl-4-(tetrahydro-2H-pyran-4-yl)phenyl)thiophene-2-carbonyl)pyrrolidin-3-yl)carbamate (150 mg, 69.45% yield) as a white solid. MS (ESI): mass calc'd. for C27H37N2O4S+ [M+1]+ m/z 485.25, found 485.2.

### Step 5 Synthesis of (S)-(3-aminopyrrolidin-1-yl)(3-methyl-5-(3-methyl-4-(tetrahydro-2H-pyran-4-yl)phenyl)thiophen-2-yl)methanone

To a solution of tert-butyl (S)-(1-(3-methyl-5-(3-methyl-4-(tetrahydro-2H-pyran-4-yl)phenyl)thiophene-2-carbonyl)pyrrolidin-3-yl)carbamate (150 mg, 0.3095 mmol) in EtOAc (2 mL) was added HCl (4 mL, 2M in EtOAc), then the mixture was stirred at 25° C. for 12 hours. After the reaction was completed, the mixture was filtered, and the filter cake was washed with EtOAc for several times and lyophilized to afford (S)-(3-aminopyrrolidin-1-yl)(3-methyl-5-(3-methyl-4-(tetrahydro-2H-pyran-4-yl)phenyl)thiophen-2-yl)methanone (Compound 161, 36.7 mg, 30.34% yield) as a white solid. MS (ESI): mass calc'd. for C22H29N2O2S+ [M+1]+ m/z 385.19, found 385.1. ¹H NMR (400 MHz, MeOD) δ 7.44-7.42 (m, 2H), 7.25 (d, J=7.6 Hz, 1H), 7.20 (s, 1H), 4.06-4.03 (m, 4H), 3.84-3.72 (m, 3H), 3.59 (t, J=11.4 Hz, 2H), 3.08-3.02 (m, 1H), 2.44-2.35 (m, 7H), 2.19-2.11 (m, 1H), 1.83-1.75 (m, 2H), 1.69-1.66 (m, 2H).

The following compounds were prepared analogously to Compound 161:

| Compound | Name | H NMR | MS |
|---|---|---|---|
| 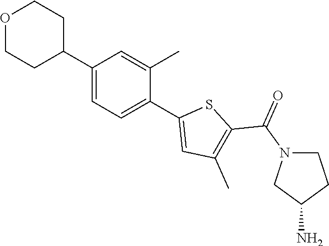 [SMILES: Cc1cc(C2CCOCC2)ccc1-c1cc(C)c(C(=O)N2CC[C@H](N)C2)s1] | (S)-(3-aminopyrrolidin-1-yl)(3- methyl-5-(2-methyl-4- (tetrahydro-2H-pyran-4- yl)phenyl)thiophen-2- yl)methanone | ¹H NMR (400 MHz, MeOD) δ 7.32 (d, J = 7.9 Hz, 1H), 7.19 (s, 1H), 7.13 (d, J = 7.9 Hz, 1H), 6.94 (s, 1H), 4.06-3.99 (m, 4H), 3.89-3.75 (m, 2H), 3.71-3.67 (m, 1H), 3.60-3.54 (m, 2H), 2.84-2.77 (m, 1H), 2.50-2.41 (m, 4H), 2.37 (s, 3H), 2.1- 2.09 (m, 1H), 1.82-1.76 (m, 4H). | 385.1 [M + H]⁺ |
| 160 | | | |
| 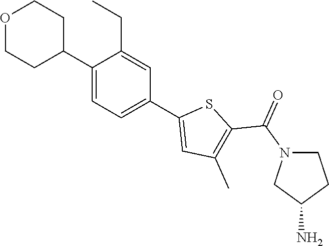 [SMILES: CCc1cc(-c2cc(C)c(C(=O)N3CC[C@H](N)C3)s2)ccc1C1CCOCC1] | (S)-(3-aminopyrrolidin-1-yl)(5- (3-ethyl-4-(tetrahydro-2H-pyran- 4-yl)phenyl)-3-methylthiophen- 2-yl)methanone | ¹H NMR (400 MHz, MeOD) δ 7.46-7.424 (m, 2H), 7.30 (d, J = 8.0 Hz, 1H), 7.22 (s, 1H), 4.08-3.98 (m, 4H), 3.89-3.75 (m, 2H), 3.73-3.67 (m, 1H), 3.60 (t, J = 11.2 Hz, 2H), 3.12-3.06 (m, 1H), 2.75 (t, J = 7.6 Hz, 1H), 2.50-2.41 (m, 1H), 2.36 (s, 3H), 2.17-2.09 (m, 1H), 1.87-1.80 (m, 2H), 1.68-1.65 (m, 2H), 1.25 (t, J = 7.6 Hz, 3H). | 399.1 [M + H]⁺ |
| 157 | | | |
| 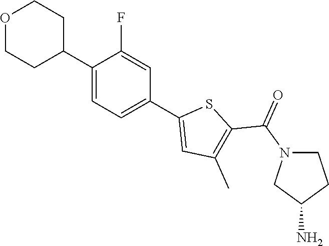 [SMILES: Cc1cc(-c2ccc(C3CCOCC3)c(F)c2)sc1C(=O)N1CC[C@H](N)C1] | (S)-(3-aminopyrrolidin-1-yl)(5- (3-fluoro-4-(tetrahydro-2H- pyran-4-yl)phenyl)-3- methylthiophen-2-yl)methanone | ¹H NMR (400 MHz, MeOD) δ 7.44-7.42 (m, 1H), 7.37-7.33 (m, 2H), 7.28 (s, 1H), 4.07-3.97 (m, 4H), 3.88-3.67 (m, 3H), 3.58 (t, J= 11.4 Hz, 2H), 3.17-3.11 (m, 1H), 2.51- 2.41 (m, 1H), 2.36 (s, 3H), 2.18-2.11 (m, 1H), 1.91-1.80 (m, 2H), 1.76-1.73 (m, 2H). | 389.1 [M + H]⁺ |
| 150 | | | |
| 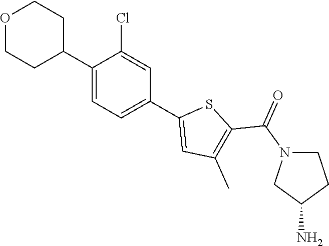 [SMILES: Cc1cc(-c2ccc(C3CCOCC3)c(Cl)c2)sc1C(=O)N1CC[C@H](N)C1] | (S)-(3-aminopyrrolidin-1-yl)(5- (3-chloro-4-(tetrahydro-2H- pyran-4-yl)phenyl)-3- methylthiophen-2-yl)methanone | ¹H NMR (400 MHz, MeOD) δ 7.66 (d, J = 1.6 Hz, 1H), 7.57 (dd, J = 8.2, 1.6 Hz, 1H), 7.40 (d, J = 8.2 Hz, 1H), 7.29 (s, 1H), 4.08- 3.97 (m, 4H), 3.88-3.68 (m, 3H), 3.63-3.57 (m, 2H), 3.36-3.33 (m, 1H), 2.50-2.41 (m, 1H), 2.36 (s, 3H), 2.20-2.13 (m, 1H), 1.81- 1.76 (m, 4H). | 405.1 [M + H]⁺ |
| 146 | | | |
| 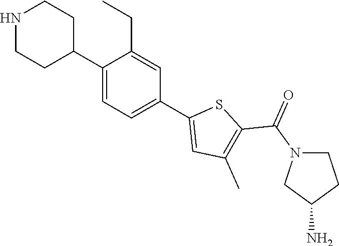 [SMILES: CCc1cc(-c2cc(C)c(C(=O)N3CC[C@H](N)C3)s2)ccc1C1CCNCC1] | (S)-(3-aminopyrrolidin-1-yl)(5- (3-ethyl-4-(piperidin-4- yl)phenyl)-3-methylthiophen-2- yl)methanone | ¹H NMR (400 MHz, MeOD) δ 7.41-7.36 (m, 2H), 7.22 (d, J = 8.0 Hz, 1H), 7.14 (s, 1H), 3.97-3.87 (m, 2H), 3.81-3.58 (m, 3H), 3.43 (d, J = 12.6 Hz, 2H), 3.17-3.06 (m, 3H), 2.68 (q, J = 7.4 Hz, 2H), 2.37 (dt, J = 13.8, 7.0 Hz, 1H), 2.26 (s,3H), 2.12-2.01 (m, 1H), 1.98-1.82 (m, 4H), 1.15 (q, J = 7.5 Hz, 3H). | 398.1 [M + H]⁺ |
| 145 | | | |
| 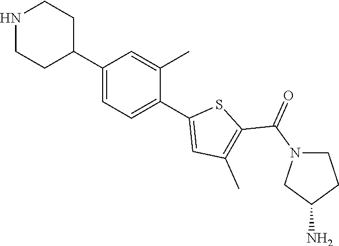 [SMILES: Cc1cc(C2CCNCC2)ccc1-c1cc(C)c(C(=O)N2CC[C@H](N)C2)s1] | (S)-(3-aminopyrrolidin-1-yl)(3- methyl-5-(2-methyl-4-(piperidin- 4-yl)phenyl)thiophen-2- yl)methanone | ¹H NMR (400 MHz, MeOD) δ 8.54 (s, 1H), 7.37 (d, J = 7.9 Hz, 1H), 7.24 (s, 1H), 7.17 (d, J = 7.9 Hz, 1H), 6.96 (s, 1H), 4.04-3.96 (m, 2H), 3.86 (dt, J = 11.6, 7.5 Hz, 1H), 3.81-3.74 (m, 1H), 3.70 (q, J = 6.6 Hz, 1H), 3.53 (d, J = 12.7 Hz, 2H), 3.16 (td, J = 12.8, 2.7 Hz, 2H), 2.93 (ddd, J = 12.1, 8.7, 3.5 Hz, 1H), 2.48-2.41 (m, 4H), 2.38 (s, 3H), 2.19-2.05 (m, 3H), 2.04-1.90 (m, 2H). | 384.1 [M + H]⁺ |
| 143 | | | |

The following compounds are prepared analogously to the methods described in this example:

| | Synthetic |
|---|---|
| Compound | procedure |
| 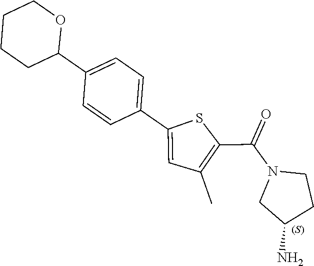 | For step 1, a corresponding tetrahydropyran carboxylic acid is used to synthesize compound 210 |
| 210 | |

### Example 65: Synthesis of (S)-(3-aminopyrrolidin-1-yl)(3-methyl-5-(4-(4-(oxetan-3-yl)piperazin-1-yl)phenyl)thiophen-2-yl)methanone (Compound 154)

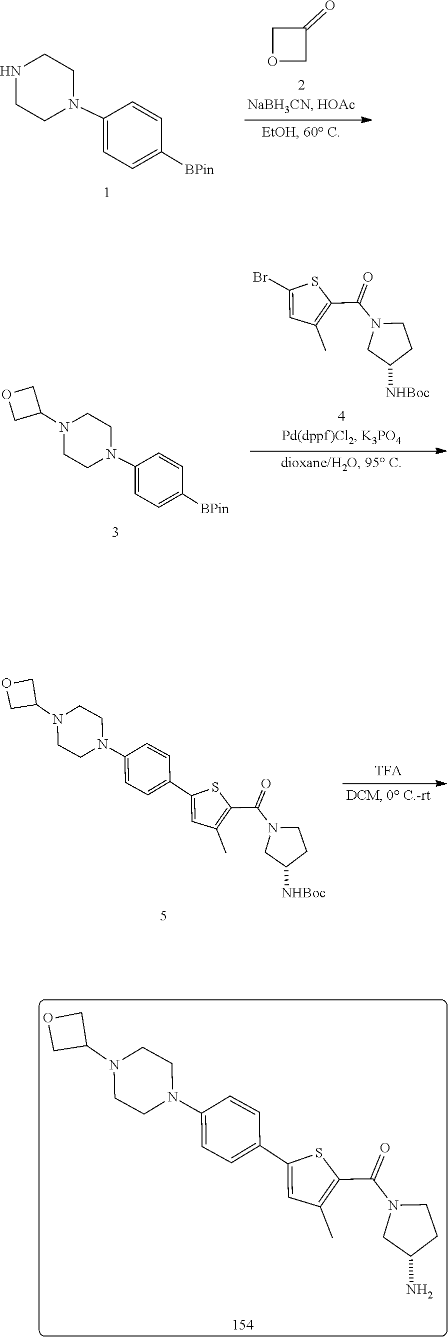

### Step 1 Synthesis of 1-(oxetan-3-yl)-4-(4-(4,4,5,5-tetramethyl-1,3,2-dioxaborolan-2-yl)phenyl)piperazine

To a solution of 1-(4-(4,4,5,5-tetramethyl-1,3,2-dioxaborolan-2-yl)phenyl)piperazine (864 mg, 3 mmol) in EtOH (20 mL) was added oxetan-3-one (84 mg, 1 mmol), NaBH₃CN (282.6 mg, 4.5 mmol) and AcOH (180 mg, 3 mmol), then the mixture was stirred at 65° C. for 16 h. The LCMS showed the reaction was completed and the desired mass was detected. After cooling to rt, the resulting mixture was concentrated under reduced pressure to give the residue, which was purified by flash chromatography (petroleum ether:ethyl acetate=9:1) to afford 1-(oxetan-3-yl)-4-(4-(4,4,5,5-tetramethyl-1,3,2-dioxaborolan-2-yl)phenyl)piperazine (600 mg, 55.2% yield) as a white solid. LCMS (ESI) calc'd for C19H29BN2O3+ [M+H]+ m/z 344.23, found 345.

### Step 2 Synthesis of tert-butyl (S)-(1-(3-methyl-5-(4-(4-(oxetan-3-yl)piperazin-1-yl)phenyl)thiophene-2-carbonyl)pyrrolidin-3-yl)carbamate

To a solution of tert-butyl (S)-(1-(5-bromo-3-methylthiophene-2-carbonyl)pyrrolidin-3-yl)carbamate (389 mg, 1 mmol) in dioxane/H₂O (v:v=8:1, 18 mL) was added 1-(oxetan-3-yl)-4-(4-(4,4,5,5-tetramethyl-1,3,2-dioxaborolan-2-yl)phenyl)piperazine (344 mg, 1 mmol), K₃PO₄ (636.3 mg, 3 mmol) and Pd(dppf)Cl₂ (163 mg, 0.2 mmol), then the mixture was stirred at 95° C. for 16 h. After cooling to rt, the mixture was filtered and concentrated under reduced pressure. The residue was purified by flash chromatography (ethyl acetate) to afford tert-butyl (S)-(1-(3-methyl-5-(4-(4-(oxetan-3-yl)piperazin-1-yl)phenyl)thiophene-2-carbonyl)pyrrolidin-3-yl)carbamate (300 mg, 54.15% yield) as a yellow solid. LCMS (ESI) calc'd for C28H38N4O4S+ [M+H]+ m/z 526.26, found 527.

### Step 3 Synthesis of (S)-(3-aminopyrrolidin-1-yl)(3-methyl-5-(4-(4-(oxetan-3-yl)piperazin-1-yl)phenyl)thiophen-2-yl)methanone

To a solution of tert-butyl N-[(3S)-1-[(3-methyl-5-{4-[4-(oxetan-3-yl)piperazin-1-yl]phenyl}thiophen-2-yl)carbonyl]pyrrolidin-3-yl]carbamate (100 mg, 0.19 mmol) in DCM (5 mL) was added TFA (324.8 mg, 2.85 mmol), then the mixture was stirred at rt for 16 h. The LCMS showed the reaction was completed and the desired mass was detected. The mixture was concentrated under reduced pressure to give the residue, which was purified by prep-HPLC to afford (S)-(3-aminopyrrolidin-1-yl)(3-methyl-5-(4-(4-(oxetan-3-yl)piperazin-1-yl)phenyl)thiophen-2-yl)methanone (Compound 154, 69.4 mg, 84.8% yield) as a white solid. LCMS (ESI) calc'd for C23H30N4O2S+ [M+H]+ m/z 426.21, found 427.

¹H NMR (400 MHz, MeOD) δ 7.60 (d, J=8.6 Hz, 2H), 7.17 (s, 1H), 7.09 (d, J=8.7 Hz, 2H), 4.97-4.92 (m, 3H), 4.87 (d, J=8.1 Hz, 2H), 4.52-4.42 (m, 1H), 4.01 (dd, J=10.4, 5.0 Hz, 2H), 3.91-3.77 (m, 2H), 3.71 (dd, J=15.3, 6.8 Hz, 1H), 3.57 (s, 3H), 3.39 (s, 4H), 2.46 (td, J=14.0, 7.4 Hz, 1H), 2.37 (s, 3H), 2.14 (td, J=12.8, 6.6 Hz, 1H).

### Example 66: Synthesis of (S)-(3-aminopyrrolidin-1-yl)(5-(3-chloro-4-(piperidin-4-yl)phenyl)-3-methylthiophen-2-yl)methanone (Compound 138)

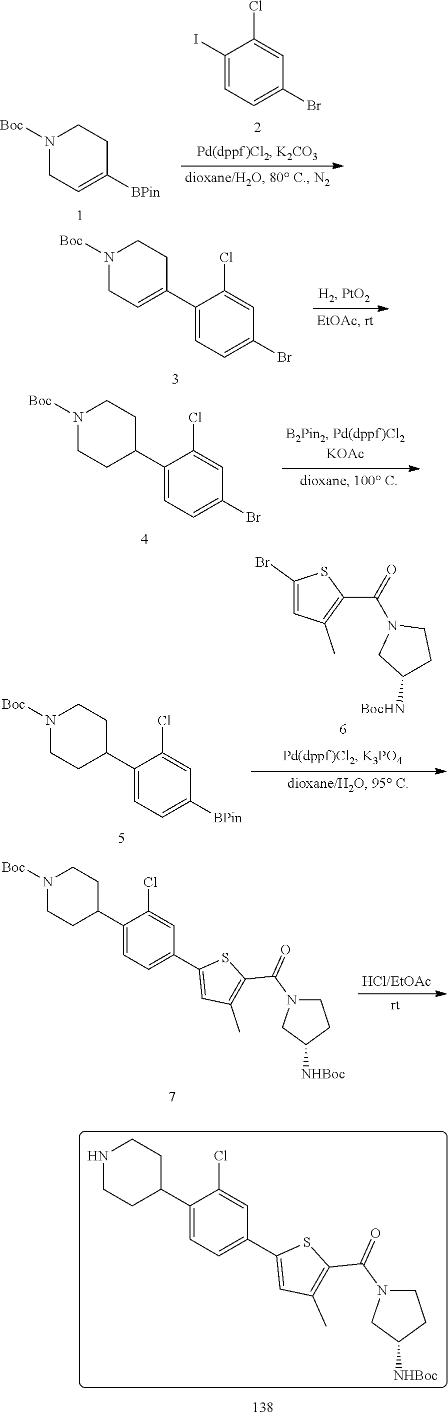

### Step 1 Synthesis of tert-butyl 4-(4-bromo-2-chlorophenyl)-3,6-dihydropyridine-1(2H)-carboxylate

To a mixture of tert-butyl [4-(4,4,5,5-tetramethyl-1,3,2-dioxaborolan-2-yl)-3,6-dihydro-2H-pyridin-1-yl]formate (1.50 g, 4.80 mmol) in 1,4-dioxane (12 mL) and H₂O (3 mL) was added 4-bromo-2-chloro-1-iodobenzene (2.28 g, 7.20 mmol), K₃PO₄ (1.99 g, 14.40 mmol) and Pd(dppf)Cl₂·DCM (0.39 g, 0.40 mmol), then the mixture was stirred at 80° C. for 16 hours under N₂ atmosphere. TLC showed the reaction was completed. The reaction mixture was concentrated under reduced pressure to give the crude product, which was purified by column chromatography on silica gel (ethyl acetate:petroleum ether=4%) to afford tert-butyl 4-(4-bromo-2-chlorophenyl)-3,6-dihydropyridine-1(2H)-carboxylate (1.00 g, 50.0% yield) as light-yellow oil. LCMS (ESI) calc'd for C16H19BrClNO2+ [M+H]+ m/z 372.03, found 372.0.

### Step 2 Synthesis of tert-butyl 4-(4-bromo-2-chlorophenyl)piperidine-1-carboxylate

A mixture of tert-butyl 4-(4-bromo-2-chlorophenyl)-3,6-dihydropyridine-1(2H)-carboxylate (1.00 g, 2.70 mmol) and PtO₂ (0.10 g, 0.40 mmol) in EtOAc (10 mL) was stirred at 25° C. for 16 hours under H₂ atmosphere. LCMS showed the reaction was completed and the desired mass was detected. The reaction mixture was filtered and the filtrate was concentrated to dryness to give the crude product, which was purified by column chromatography on silica gel (ethyl acetate. petroleum ether=4%) to afford tert-butyl 4-(4-bromo-2-chlorophenyl)piperidine-1-carboxylate (0.57 g, 51.9% yield) as colorless oil. LCMS (ESI) calc'd for C16H21BrClNO2+ [M+H]+ m/z 374.04, found 374.0.

### Step 3 Synthesis of tert-butyl 4-(2-chloro-4-(4,4,5,5-tetramethyl-1,3,2-dioxaborolan-2-yl)phenyl)piperidine-1-carboxylate

To a mixture of tert-butyl 4-(4-bromo-2-chlorophenyl)piperidine-1-carboxylate (570 mg, 1.52 mmol) in 1,4-dioxane (10 mL) was added B₂Pin₂ (462 mg, 1.82 mmol), potassium acetate (447 mg, 4.55 mmol) and Pd(dppf)Cl₂·DCM (186 mg, 0.23 mmol), then it was stirred at 100° C. for 16 hours under N₂ atmosphere. LCMS showed the reaction was completed and the desired mass was detected. The reaction mixture was concentrated under reduced pressure to give the crude product, which was purified by column chromatography on silica gel (ethyl acetate:petroleum ether=4%) to afford tert-butyl 4-(2-chloro-4-(4,4,5,5-tetramethyl-1,3,2-dioxaborolan-2-yl)phenyl)piperidine-1-carboxylate (455 mg, 63.9% yield) as colorless oil. LCMS (ESI) calc'd for C22H33BClNO4+ [M+H]+ m/z 422.22, found 422.1.

### Step 4 Synthesis of tert-butyl (S)-4-(4-(5-(3-((tert-butoxycarbonyl)amino)pyrrolidine-1-carbonyl)-4-methylthiophen-2-yl)-2-chlorophenyl)piperidine-1-carboxylate

To a solution of tert-butyl 4-(2-chloro-4-(4,4,5,5-tetramethyl-1,3,2-dioxaborolan-2-yl)phenyl)piperidine-1-carboxylate (163 mg, 0.39 mmol) and compound 6 (150 mg, 0.39 mmol) in 1,4-dioxane (5 mL) and H₂O (1 mL) was added K₃PO₄ (245 mg, 1.16 mmol) and Pd(dppf)Cl₂ (47 mg, 0.06 mmol), then the mixture was stirred at 95° C. for 16 hours under N₂ atmosphere. LCMS showed the reaction was completed and the desired mass was detected. The reaction mixture was concentrated under reduced pressure to give the crude product, which was purified by column chromatography on silica gel (ethyl acetate:petroleum ether=32%) to afford tert-butyl (S)-4-(4-(5-(3-((tert-butoxycarbonyl)amino)pyrrolidine-1-carbonyl)-4-methylthiophen-2-yl)-2-chlorophenyl)piperidine-1-carboxylate (160 mg, 65.2%) as colorless oil. LCMS (ESI) calc'd for C31H42ClN₃O5S+ [M+H]+ m/z 604.25, found 604.1.

### Step 5 Synthesis of (S)-(3-aminopyrrolidin-1-yl)(5-(3-chloro-4-(piperidin-4-yl)phenyl)-3-methylthiophen-2-yl)methanone

A mixture of tert-butyl (S)-4-(4-(5-(3-((tert-butoxycarbonyl)amino)pyrrolidine-1-carbonyl)-4-methylthiophen-2-yl)-2-chlorophenyl)piperidine-1-carboxylate (160 mg, 0.26 mmol) in HCl (2 M in EtOAc, 3 mL) was stirred at 25° C. for 16 hours. LCMS showed the reaction was completed and the desired mass was detected. The reaction mixture was concentrated under reduced pressure to give the residue, which was purified by prep-HPLC to afford (S)-(3-aminopyrrolidin-1-yl)(5-(3-chloro-4-(piperidin-4-yl)phenyl)-3-methylthiophen-2-yl)methanone (Compound 138, 52.5 mg, 48.7%) as a white solid. MS (ESI): mass calc'd. for C21H26ClN3OS+ [M+H]+ m/z 404.15, found 404.1. ¹H NMR (400 MHz, MeOD) δ 7.71 (d, J=2.0 Hz, 1H), 7.61 (dd, J=8.4, 2.0 Hz, 1H), 7.40 (d, J=8.0 Hz, 1H), 7.31 (s, 1H), 4.02-3.95 (m, 2H), 3.88-3.80 (m, 1H), 3.80-3.73 (m, 1H), 3.72-3.65 (m, 1H), 3.54 (d, J=12.4 Hz, 2H), 3.46-3.37 (m, 1H), 3.25-3.16 (m, 2H), 2.50-2.40 (m, 1H), 2.36 (s, 3H), 2.17-2.07 (m, 3H), 2.00-1.87 (m, 2H).

The following compound was prepared analogously to Compound 138:

| Compound | Name | H NMR | MS |
|---|---|---|---|
| 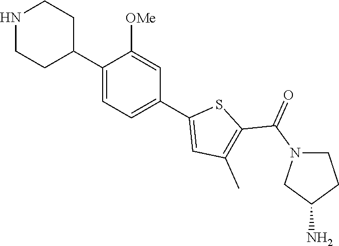 [SMILES: COc1cc(-c2cc(C)c(C(=O)N3CC[C@H](N)C3)s2)ccc1C1CCNCC1] | (S)-(3- aminopyrrolidin-1- yl)(5-(3-methoxy- 4-(piperidin-4- yl)phenyl)-3- methylthiophen-2- yl)methanone | ¹H NMR (400 MHz, MeOD) δ 7.28 (s, 1H), 7.24 (d, J = 8.0 Hz, 2H), 7.20 (s, 1H), 4.04- 3.97 (m, 2H), 3.94-3.90 (m, 3H), 3.89-3.76 (m, 2H), 3.75- 3.67 (m, 1H), 3.54-3.47 (m, 2H), 3.30-3.24 (m, 1H), 3.20- 3.11 (m, 2H), 2.50-2.41 (m, 1H), 2.37 (s, 3H), 2.19-2.10 (m, 1H), 2.08-1.90 (m, 4H). | 400.4 [M + H]⁺ |
| 130 | | | |

### Example 67: Synthesis of (S)-(3-aminopyrrolidin-1-yl)(5-(3-ethyl-4-(1-methylpiperidin-4-yl)phenyl)-3-methylthiophen-2-yl)methanone (Compound 137)

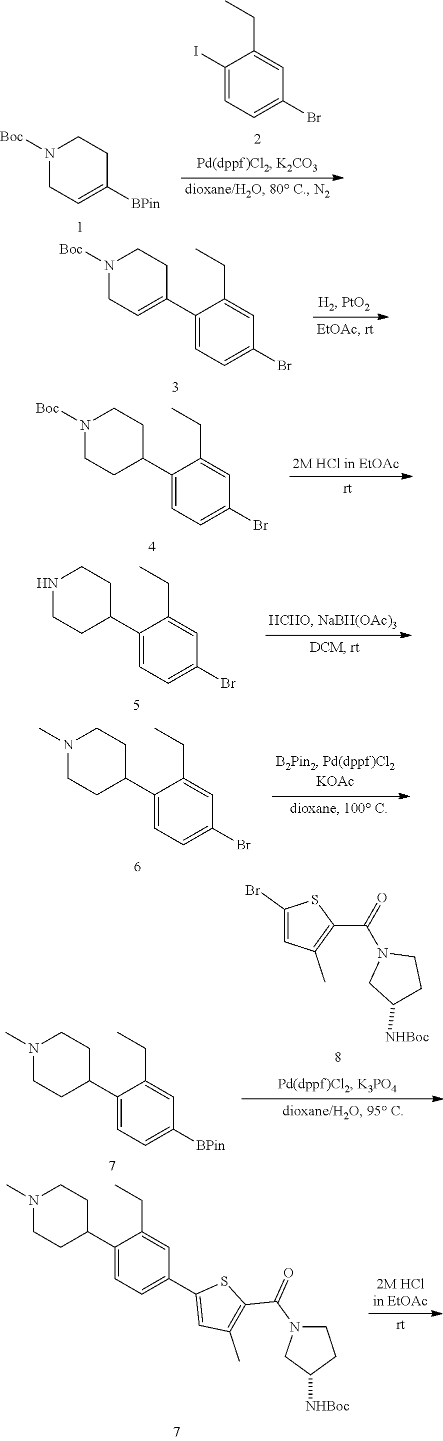

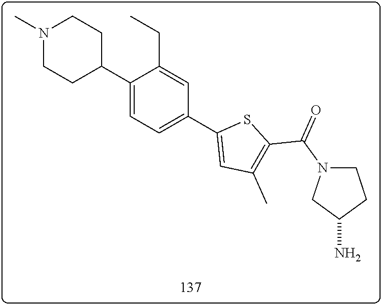 [SMILES: CCc1cc(-c2cc(C)c(C(=O)N3CC[C@H](N)C3)s2)ccc1C1CCN(C)CC1]

### Step 1 Synthesis of tert-butyl 4-(4-bromo-2-ethylphenyl)-3,6-dihydropyridine-1(2H)-carboxylate (3)

To a solution of 4-bromo-2-ethyl-1-iodobenzene (1.0 g, 3.2 mmol) in dioxane/H₂O (v v=8:1, 18 mL) was added tert-butyl [4-(4,4,5,5-tetramethyl-1,3,2-dioxaborolan-2-yl)-3,6-dihydro-2H-pyridin-1-yl]formate (997.6 mg, 3.2 mmol), K₂CO₃ (1.33 g, 9.65 mmol) and Pd(dppf)Cl₂ (524.8 mg, 0.64 mmol), then the mixture was stirred at 95° C. under nitrogen for 16 h. After cooling to rt, the mixture was filtered and concentrated under reduced pressure to give the residue, which was purified by flash chromatography (petroleum ether:ethyl acetate=5:1) to afford tert-butyl 4-(4-bromo-2-ethylphenyl)-3,6-dihydropyridine-1(2H)-carboxylate (870 mg, 70% yield) as yellow oil. LCMS (ESI) calc'd for C18H24BrNO2+ [M+H]+ m/z 366.10, found 366.

### Step 2 Synthesis of tert-butyl 4-(4-bromo-2-ethylphenyl)piperidine-1-carboxylate

To a solution of tert-butyl 4-(4-bromo-2-ethylphenyl)-3,6-dihydropyridine-1(2H)-carboxylate (870 mg, 2.22 mmol) in EtOAc (10 mL) was added PtO₂ (50.38 mg, 0.22 mmol), then the mixture was stirred at rt under hydrogen for 16 h. The LCMS showed the reaction was completed and the desired mass was detected. The resulting mixture was filtered and concentrated under reduced pressure to afford tert-butyl 4-(4-bromo-2-ethylphenyl)piperidine-1-carboxylate (855 mg, 93% yield) as yellow oil. LCMS (ESI) calc'd for C18H26BrNO2+ [M+H]+ m/z 368.11, found 368.

### Step 3 Synthesis of 4-(4-bromo-2-ethylphenyl)piperidine

A solution of [4-(4-bromo-2-ethylphenyl)piperidin-1-yl]tert-butyl formate (5.86 g, 2.3 mmol) in HCl (2 M in EA, 5 mL) was stirred at rt for 16 h. The LCMS showed the reaction was completed and the desired mass was detected. The resulting mixture was concentrated under reduced pressure to afford 4-(4-bromo-2-ethylphenyl)piperidine (750 mg, 96.6% yield) as yellow oil. LCMS (ESI) calc'd for C13H18BrN+ [M+H]+ m/z 268.06, found 268.

### Step 4 Synthesis of 4-(4-bromo-2-ethylphenyl)-1-methylpiperidine

To a solution of 4-(4-bromo-2-ethylphenyl)piperidine (750 mg, 2.9 mmol) in DCM (10 mL) was added formaldehyde (177 mg, 5.9 mmol) and sodium triacetoxyborohydride (3.13 g, 14.7 mmol), then the mixture was stirred at rt for 16 h. The LCMS showed the reaction was completed and the desired mass was detected. The mixture was concentrated under reduced pressure to give the residue, which was purified by flash chromatography (petroleum ether:ethyl acetate=10:1) to afford 4-(4-bromo-2-ethylphenyl)-1-methylpiperidine (230 mg, 27.6% yield) as yellow oil. LCMS (ESI) calc'd for C13H18BrN+[M+H]+ m/z 268.06, found 268.

### Step 5 Synthesis of 4-(2-ethyl-4-(4,4,5,5-tetramethyl-1,3,2-dioxaborolan-2-yl)phenyl)-1-methylpiperidine

To a solution of 4-(4-bromo-2-ethylphenyl)-1-methylpiperidine (230 mg, 0.82 mmol) in dioxane (10 mL) was added 4,4,5,5-tetramethyl-2-(4,4,5,5-tetramethyl-1,3,2-dioxaborolan-2-yl)-1,3,2-dioxaborolane (207 mg, 0.82 mmol), potassium acetate (240 mg, 2.4 mmol) and Pd(dppf)Cl₂ (133 mg, 0.16 mmol), then the mixture was stirred at 95° C. for 16 h. After cooling to rt, the mixture was filtered and concentrated under reduced pressure to give the residue, which was purified by flash chromatography (petroleum ether:ethyl acetate=10:1) to afford 4-(2-ethyl-4-(4,4,5,5-tetramethyl-1,3,2-dioxaborolan-2-yl)phenyl)-1-methylpiperidine (180 mg, 63.7% yield) as a yellow solid. LCMS (ESI) calc'd for C20H32BNO2+ [M+H]+ m/z 330.25, found 330.

### Step 6 Synthesis of tert-butyl (S)-(1-(5-(3-ethyl-4-(1-methylpiperidin-4-yl)phenyl)-3-methylthiophene-2-carbonyl)pyrrolidin-3-yl)carbamate

To a solution of tert-butyl (S)-(1-(5-bromo-3-methylthiophene-2-carbonyl)pyrrolidin-3-yl)carbamate (180 mg, 0.46 mmol) in dioxane/H₂O (v:v=8:1, 18 mL) was added 4-(2-ethyl-4-(4,4,5,5-tetramethyl-1,3,2-dioxaborolan-2-yl)phenyl)-1-methylpiperidine (152.3 mg, 0.46 mmol), K₃PO₄ (294.5 mg, 1.39 mmol) and Pd(dppf)Cl₂ (75.5 mg, 0.09 mmol), then the mixture was stirred at 95° C. for 16 h. After cooling to rt, the mixture was filtered and concentrated under reduced pressure to give the residue, which was purified by flash chromatography (petroleum ether:ethyl acetate=5:1) to afford tert-butyl (S)-(1-(5-(3-ethyl-4-(1-methylpiperidin-4-yl)phenyl)-3-methylthiophene-2-carbonyl)pyrrolidin-3-yl)carbamate (137 mg, 55% yield) as yellow oil. LCMS (ESI) calc'd for C29H41N3O3S+[M+H]+ m/z 512.29, found 512.

### Step 7 Synthesis of (S)-(3-aminopyrrolidin-1-yl)(5-(3-ethyl-4-(1-methylpiperidin-4-yl)phenyl)-3-methylthiophen-2-yl)methanone

A solution of tert-butyl (S)-(1-(5-(3-ethyl-4-(1-methylpiperidin-4-yl)phenyl)-3-methylthiophene-2-carbonyl)pyrrolidin-3-yl)carbamate (137 mg, 0.27 mmol) in HCl (2M in EA, 3 mL) was stirred at rt for 16 h. The LCMS showed the reaction was completed and the desired mass was detected. The mixture was concentrated under reduced pressure to give the residue, which was purified by prep-HPLC to afford (S)-(3-aminopyrrolidin-1-yl)(5-(3-ethyl-4-(1-methylpiperidin-4-yl)phenyl)-3-methylthiophen-2-yl)methanone (Compound 137, 6.1 mg, 5.3% yield) as a yellow solid. LCMS (ESI) calc'd for C23H31N3OS+ [M+H]+ m/z 398.22, found 398. ¹H NMR (400 MHz, MeOD) δ 7.37 (d, J=6.0 Hz, 2H), 7.17 (d, J=8.6 Hz, 1H), 7.12 (s, 1H), 3.94-3.84 (m, 2H), 3.75 (dt, J=11.8, 7.5 Hz, 1H), 3.68-3.58 (m, 2H), 3.52 (d, J=12.2 Hz, 2H), 3.17-3.00 (m, 2H), 2.82 (s, 3H), 2.66 (q, J=7.5 Hz, 2H), 2.34 (dt, J=13.9, 7.1 Hz, 1H), 2.25 (s, 3H), 2.09-1.96 (m, 2H), 1.91 (t, J=11.5 Hz, 3H), 1.20-1.08 (m, 3H).

### Example 68: Synthesis of (S)-(3-aminopyrrolidin-1-yl)(5-(4-(4-(dimethylamino)piperidin-4-yl)phenyl)-3-methylthiophen-2-yl)methanone (Compound 133)

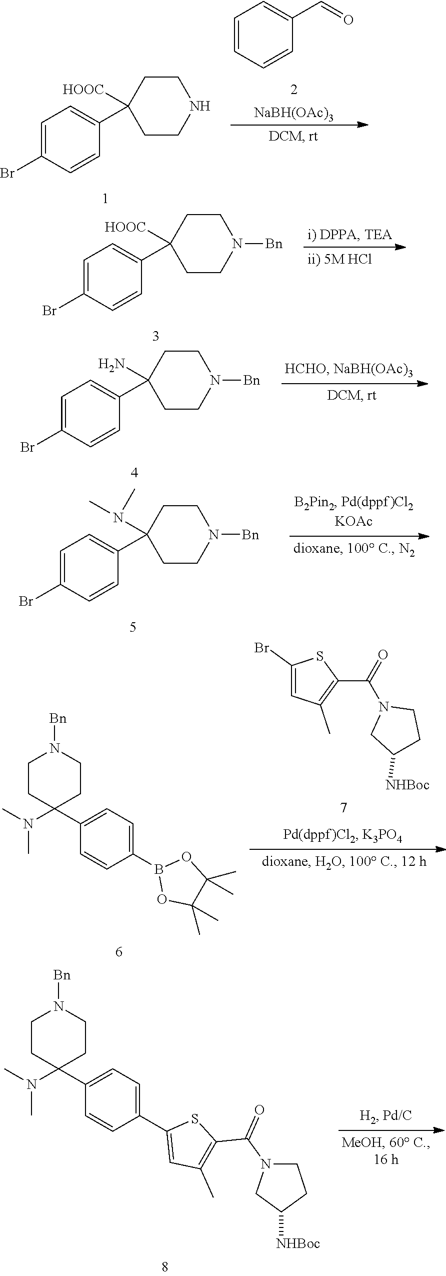

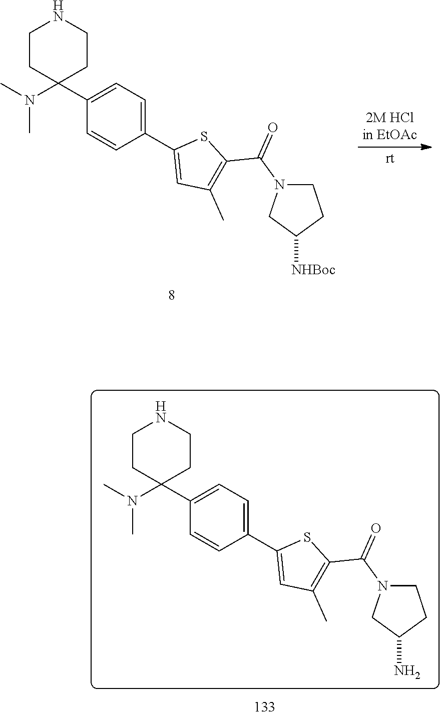 [SMILES: Cc1cc(-c2ccc(C3(N(C)C)CCNCC3)cc2)sc1C(=O)N1CC[C@H](NC(=O)OC(C)(C)C)C1 ; Cc1cc(-c2ccc(C3(N(C)C)CCNCC3)cc2)sc1C(=O)N1CC[C@H](N)C1]

### Step 1 Synthesis of 1-benzyl-4-(4-bromophenyl)piperidine-4-carboxylic acid

To the solution of 4-(4-bromophenyl) piperidine-4-carboxylic acid (1.5 g, 5.3 mmol) in DCM (30 mL) was added benzaldehyde (1.69 g, 15.9 mmol), sodium triacetoxyborohydride (1.68 g, 7.9 mmol) and HOAc (0.64 g, 10.6 mmol), then the mixture was stirred at rt for 16 h. The LCMS showed the reaction was completed and the desired mass was detected. The mixture was concentrated to give the residue, which was purified by silica gel column chromatography to give the desired product (0.85 g, 37.74% yield) as a white solid. LCMS (ESI) calc'd for C19H20BrNO2+ [M+H]+ m/z 374.07, found 374.1.

### Step 2 Synthesis of 1-benzyl-4-(4-bromophenyl)piperidin-4-amine

To a solution of 4-(4-bromophenyl)-1-(1-methylphenyl) piperidine-4-carboxylic acid (850 mg, 2.27 mmol) in toluene (15 mL) was added TEA (504.23 mg, 5.0 mmol) and DPPA (685.66 mg, 2.50 mmol) via a syringe under nitrogen and the mixture was stirred at 90° C. for 2 h. After completion of the reaction, the mixture was cooled to room temperature and diluted with EtOAc. The mixture was washed with saturated aq. NaHCO₃ twice and aq. NaCl once, then dried over anhydrous Na₂SO₄, filtered and concentrated under reduced pressure to give a residue, which was dissolved with HCl (5 M in water, 6 mL) and stirred at 100° C. for 5 h. After the mixture was cooled to room temperature, toluene was added and the solution was concentrated under reduced pressure to give a solid, which was dissolved in saturated aq. NaHCO₃ (60 mL) and EtOAc (60 mL). The combined organic layer was washed with saturated aq. NaHCO₃ four times, dried over anhydrous Na₂SO₄ and evaporated in vacuum to give the crude product, which was purified by silica gel column chromatography to give 4-(4-bromophenyl)-1-(1-methylphenyl) piperidin-4-amine (390 mg, 44.75% yield) as a yellow oil. LCMS (ESI) calc'd for C18H21BrN2+ [M+H]+ m/z 345.09, found 345.0.

### Step 3 Synthesis of 1-benzyl-4-(4-bromophenyl)-N,N-dimethylpiperidin-4-amine

To a solution of 4-(4-bromophenyl)-1-(1-methylphenyl) piperidin-4-amine (390 mg, 1.13 mmol) in DCM (30 mL) was added 37% HCHO (280 mg, 3.38 mmol), Na₂SO₄ (96.0 mg, 0.68 mmol) and sodium triacetoxyborohydride (1.43 g, 6.76 mmol), then the mixture was stirred at rt for 16 h. The LCMS showed the reaction was completed and the desired mass was detected. The resulting mixture was added EtOAc (20 mL) and evaporated in vacuum, then NaHCO₃ (30 mL) and EtOAc (30 mL) were added. The aqueous layer was extracted with EtOAc (2*50 mL), and the combined organic phase was dried over anhydrous Na₂SO₄, filtered and evaporated in vacuum to give the crude product, which was purified by silica gel column chromatography to afford 4-(4-bromophenyl)-N,N-dimethyl-1-(1-methylphenyl)piperidin-4-amine (340 mg, 72.58% yield) as yellow oil. LCMS (ESI) calc'd for C20H25BrN2+ [M+H]+ m/z 373.12, found 373.1.

### Step 4 Synthesis of 1-benzyl-N,N-dimethyl-4-(4-(4,4,5,5-tetramethyl-1,3,2-dioxaborolan-2-yl)phenyl)piperidin-4-amine

To a solution of 4-(4-bromophenyl)-NN-dimethyl-1-(1-methylphenyl) piperidin-4-amine (340 mg, 0.9083 mmol) in 1,4-dioxane (8 mL) was added B₂Pin₂ (253.72 mg, 0.9991 mmol), Pd(dppf)Cl₂·DCM (148.23 mg, 0.1816 mmol) and potassium acetate (267.43 mg, 2.7249 mmol), then the mixture was stirred under N₂ at 95° C. for 16 h. The LCMS showed the reaction was completed and the desired mass was detected. After cooling to rt, the mixture was filtered and concentrated under reduced pressure to give the residue, which was purified by silica gel column chromatography to afford the desired product (300 mg, 70.54% yield) as a yellow solid. LCMS (ESI) calc'd for C26H37BN2O2+ [M+H]+ m/z 421.29, found 421.2.

### Step 5 Synthesis of tert-butyl (S)-(1-(5-(4-(1-benzyl-4-(dimethylamino)piperidin-4-yl)phenyl)-3-methylthiophene-2-carbonyl)pyrrolidin-3-yl)carbamate

To a solution of 1-benzyl-N,N-dimethyl-4-(4-(4,4,5,5-tetramethyl-1,3,2-dioxaborolan-2-yl)phenyl)piperidin-4-amine (300 mg, 0.7119 mmol) in dioxane/H₂O (v:v=8:1, 9 mL) was added tert-butyl N-((3S)-1-((5-bromo-3-methylthiophen-2-yl)carbonyl)pyrrolidin-3-yl)carbamate (277.2 mg, 0.71 mmol), K₃PO₄ (453.4 mg, 2.14 mmol) and Pd(dppf)Cl₂·DCM (116.2 mg, 0.14 mmol), then the mixture was stirred under N₂ at 95° C. for 16 h. The LCMS showed the reaction was completed and the desired mass was detected. After cooling to rt, the mixture was filtered and concentrated under reduced pressure to give the residue, which was purified by silica gel column chromatography to afford tert-butyl (S)-(1-(5-(4-(1-benzyl-4-(dimethylamino)piperidin-4-yl)phenyl)-3-methylthiophene-2-carbonyl)pyrrolidin-3-yl)carbamate (300 mg, 62.80% yield) as a yellow solid. LCMS (ESI) calc'd for C35H46N4O3S+ [M+H]+ m/z 603.33, found 603.3.

### Step 6 Synthesis of tert-butyl (S)-(1-(5-(4-(4-(dimethylamino)piperidin-4-yl)phenyl)-3-methylthiophene-2-carbonyl)pyrrolidin-3-yl)carbamate

To the solution of tert-butyl (S)-(1-(5-(4-(1-benzyl-4-(dimethylamino)piperidin-4-yl)phenyl)-3-methylthiophene-2-carbonyl)pyrrolidin-3-yl)carbamate (300 mg, 0.50 mmol) in MeOH (3 mL) was added 10% Pd/C (30 mg), then the mixture was stirred under hydrogen at 60° C. for 16 h. The LCMS showed the reaction was completed and the desired mass was detected. The mixture was concentrated under reduced pressure to give the residue, which was purified by prep-HPLC to afford tert-butyl (S)-(1-(5-(4-(4-(dimethylamino)piperidin-4-yl)phenyl)-3-methylthiophene-2-carbonyl)pyrrolidin-3-yl)carbamate (70 mg, 24.74% yield) as a yellow solid.

LCMS (ESI) calc'd for C28H40N4O3S+ [M+H]+ m/z 513.28, found 513.2.

### Step 7 Synthesis of (S)-(3-aminopyrrolidin-1-yl)(5-(4-(4-(dimethylamino)piperidin-4-yl)phenyl)-3-methylthiophen-2-yl)methanone

To a solution of tert-butyl (S)-(1-(5-(4-(4-(dimethylamino)piperidin-4-yl)phenyl)-3-methylthiophene-2-carbonyl)pyrrolidin-3-yl)carbamate (70 mg, 0.1365 mmol) in EtOAc (2 mL) was added HCl (2M in EA, 5 mL), then the mixture was stirred at rt for 16 h. The LCMS showed the reaction was completed and the desired mass was detected. The mixture was concentrated under reduced pressure to give the residue, which was purified by prep-HPLC to afford (S)-(3-aminopyrrolidin-1-yl)(5-(4-(4-(dimethylamino)piperidin-4-yl)phenyl)-3-methylthiophen-2-yl)methanone (Compound 133, 15 mg, 23.96% yield) as a yellow solid. MS (ESI): calc'd for C23H32N4OS+ [M+H]+ m/z 413, found 413. ¹H NMR (400 MHz, MeOD) δ 7.93 (d, J=8.0 Hz, 2H), 7.80 (d, J=8.4 Hz, 2H), 7.45 (s, 1H), 4.05-3.98 (m, 2H), 3.87-3.75 (m, 2H), 3.70 (d, J=8.4 Hz, 1H), 3.59 (d, J=13.2 Hz, 2H), 3.30-3.27 (m, 2H), 2.83 (t, J=13.2 Hz, 2H), 2.72 (s, 6H), 2.57-2.43 (m, 3H), 2.38 (s, 3H), 2.19-2.11 (m, 1H).

The following compound was prepared analogously to Compound 133:

| Compound | Name | H NMR | MS |
|---|---|---|---|
| 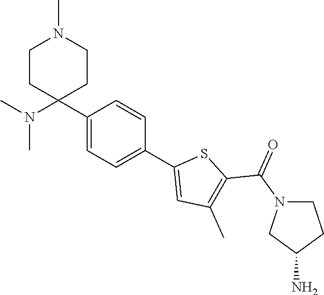 [SMILES: Cc1cc(-c2ccc(C3(N(C)C)CCN(C)CC3)cc2)sc1C(=O)N1CC[C@H](N)C1] | (S)-(3- aminopyrrolidin-1- yl)(5-(6-(1- isopropylpiperidin- 4-yl)pyridin-3-yl)- 3-methylthiophen- 2-yl)methanone | ¹H NMR (400 MHz, MeOD) δ 7.84 (d, J = 8.0 Hz, 2H), 7.70 (d, J = 8.4 Hz, 2H), 7.35 (s, 1H), 3.91 (d, J = 8.0 Hz, 2H), 3.78-3.66 (m, 2H), 3.62-3.55 (m, 3H), 3.27 (d, J = 14.0 Hz, 2H), 2.77 (t, J = 13.2Hz, 2H), 2.71 (d, J = 8.8 Hz, 3H), 2.62 (s, 6H), 2.52 (d, J = 12.4 Hz, 2H), 2.36 (s, 1H), 2.28 (s, 3H), 2.15-1.98 (m, 2H), 1.96-1.90 (m, 1H). | 427.1 [M + H]⁺ |
| 132 | | | |

### Example 69: Synthesis of 2-(2-methoxyethoxy)ethyl (S)-(4-(5-(3-aminopyrrolidine-1-carbonyl)-4-methylthiophen-2-yl)benzyl)(methyl)carbamate (Compound 222)

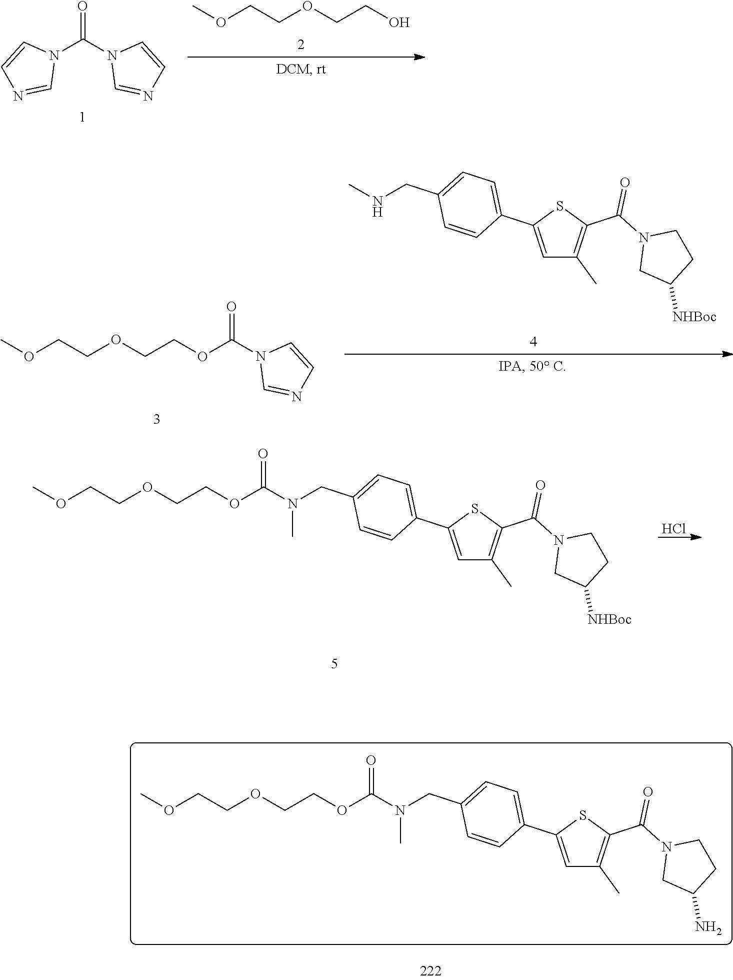

### Step 1 Synthesis of 2-(2-methoxyethoxy)ethyl 1H-imidazole-1-carboxylate

To a solution of 2-(2-methoxyethoxy)ethanol (2 g, 16.6 mmol), di(1H-imidazol-1-yl)methanone (2.96 g, 18.2 mmol) in DCM (20 mL), the mixture was stirred at 25° C. for 16 hrs. After the reaction, the solvent was removed under reduced pressure to afford 2-(2-methoxyethoxy)ethyl 1H-imidazole-1-carboxylate (2.1 g, 59.04% yield) as a yellowish oil. LCMS (ESI) calculated for C9H14N2O4+ [M+H]+ m/z 215.10, found 214.95.

### Step 2 Synthesis of 2-(2-methoxyethoxy)ethyl (S)-(4-(5-(3-((tert-butoxycarbonyl)amino)pyrrolidine-1-carbonyl)-4-methylthiophen-2-yl)benzyl)(methyl)carbamate

To a solution of 2-(2-methoxyethoxy)ethyl 1H-imidazole-1-carboxylate (200 mg, 0.9336 mmol) and tert-butyl (S)-(1-(3-methyl-5-(4-((methylamino)methyl)phenyl)thiophene-2-carbonyl)pyrrolidin-3-yl)carbamate (521 mg, 1.2136 mmol), the mixture was stirred at 50° C. for 16 hrs. After the reaction, the mixture was diluted with H₂O and the resulted mixture was extracted with EtOAc for 3 times. Combined with EtOAc phases, washed with brine, dried over Na₂SO₄ and filtered, the solvent was removed under reduced pressure and the residue was purified by flash column chromatography using petroleum ether/EtOAc as eluent to afford 2-(2-methoxyethoxy)ethyl (S)-(4-(5-(3-((tert-butoxycarbonyl)amino)pyrrolidine-1-carbonyl)-4-methylthiophen-2-yl)benzyl)(methyl)carbamate (180 mg, 33.49% yield) as yellow oil.

LCMS (ESI) calculated for C29H41N3O7S+ [M+H]+ m/z 576.27, found 576.00.

### Step 3 Synthesis of 2-(2-methoxyethoxy)ethyl (S)-(4-(5-(3-aminopyrrolidine-1-carbonyl)-4-methylthiophen-2-yl)benzyl)(methyl)carbamate

To a solution of 2-(2-methoxyethoxy)ethyl (S)-(4-(5-(3-((tert-butoxycarbonyl)amino)pyrrolidine-1-carbonyl)-4-methylthiophen-2-yl)benzyl)(methyl)carbamate (100 mg, 0.1737 mmol) in EtOAc (2 mL) was added HCl (4 M in 1,4-dioxane, 5 mL), then the mixture was stirred at 25° C. for 16 hrs. After the completion, the solvent was removed under reduced pressure to give the residue, which was purified by prep-HPLC to afford 2-(2-methoxyethoxy)ethyl (S)-(4-(5-(3-aminopyrrolidine-1-carbonyl)-4-methylthiophen-2-yl)benzyl)(methyl)carbamate (Compound 222, 28 mg, 33.91% yield) as a white solid. LCMS (ESI) calculated for C24H33N3O5S+ [M+H]+ m/z 476.21, found 475.90.

¹H NMR (400 MHz, MeOD) δ 7.62 (d, J=8.2 Hz, 2H), 7.32 (d, J=7.3 Hz, 2H), 7.26 (s, 1H), 4.51 (d, J=5.7 Hz, 2H), 4.32-4.21 (m, 2H), 3.98 (t, J=7.2 Hz, 2H), 3.88-3.75 (m, 2H), 3.71 (dd, J=15.2, 8.7 Hz, 3H), 3.57 (dd, J=38.9, 16.7 Hz, 4H), 3.33 (d, J=12.6 Hz, 3H), 2.90 (s, 3H), 2.48-2.40 (m, 1H), 2.35 (s, 3H), 2.16-2.08 (m, 1H).

### Example 70: Synthesis of (S)-(3-aminopyrrolidin-1-yl)(5-(5-(1-isopropylpiperidin-4-yl)pyridin-2-yl)-3-methylthiophen-2-yl)methanone (Compound 127)

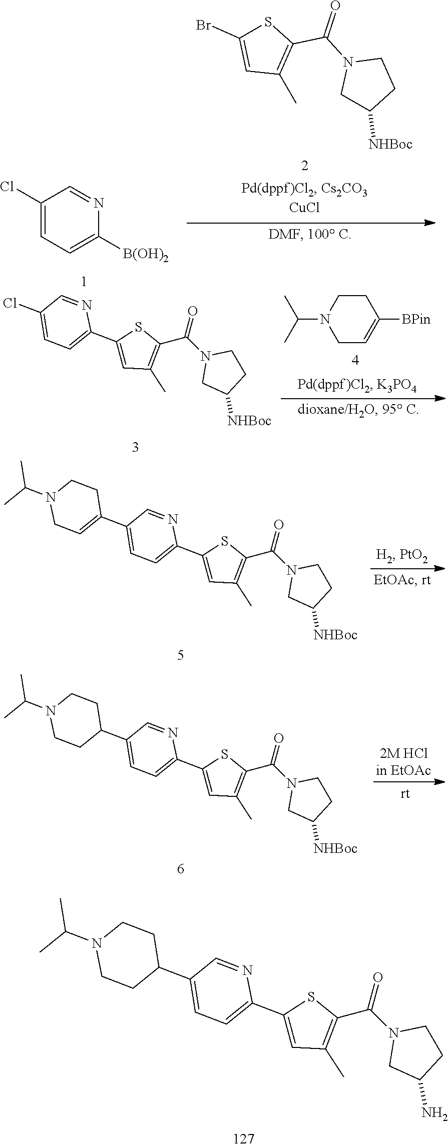

### Step 1 Synthesis of tert-butyl (S)-(1-(5-(5-chloropyridin-2-yl)-3-methylthiophene-2-carbonyl)pyrrolidin-3-yl)carbamate

To a solution of tert-butyl (S)-(1-(5-bromo-3-methylthiophene-2-carbonyl)pyrrolidin-3-yl)carbamate (390 mg, 1.0 mmol) in DMF (10 mL) was added (5-chloropyridin-2-yl)boranediol (157.6 mg, 1.0 mmol), Cs₂CO₃ (979.2 mg, 3.0 mmol), Pd(dppf)Cl₂·DCM (163.5 mg, 0.2 mmol) and CuCl (99 mg, 1.0 mmol), then the mixture was stirred at 100° C. for 16 h. After cooling to rt, the mixture was filtered and concentrated under reduced pressure to give the residue, which was purified by flash chromatography (petroleum ether:ethyl acetate=1:1) to afford tert-butyl (S)-(1-(5-(5-chloropyridin-2-yl)-3-methylthiophene-2-carbonyl)pyrrolidin-3-yl)carbamate (85 mg, 19.11% yield) as a yellow solid. LCMS (ESI) calc'd for C20H24ClN3O3S+ [M+H]+ m/z 422.12, found 422.

### Step 2 Synthesis of tert-butyl (S)-(1-(5-(1′-isopropyl-1′,2′,3′,6′-tetrahydro-[3,4′-bipyridin]-6-yl)-3-methylthiophene-2-carbonyl)pyrrolidin-3-yl)carbamate

To a solution of tert-butyl (S)-(1-(5-(5-chloropyridin-2-yl)-3-methylthiophene-2-carbonyl)pyrrolidin-3-yl)carbamate (480 mg, 1.14 mmol) in dioxane/H₂O (v:v=8:1, 18 mL) was added 1-isopropyl-4-(4,4,5,5-tetramethyl-1,3,2-dioxaborolan-2-yl)-3,6-dihydro-2H-pyridine (285.7 mg, 1.14 mmol), K₃PO₄ (724.4 mg, 3.41 mmol) and Pd(dppf)Cl₂ (185.7 mg, 0.23 mmol), then the mixture was stirred at 95° C. under N₂ for 16 h. After cooling to rt, the mixture was filtered and concentrated under reduced pressure to give the residue, which was purified by flash chromatography (ethyl acetate:methanol=10:1) to afford tert-butyl (S)-(1-(5-(1′-isopropyl-1′,2′,3′,6′-tetrahydro-[3,4′-bipyridin]-6-yl)-3-methylthiophene-2-carbonyl)pyrrolidin-3-yl)carbamate (480 mg, 66.1% yield) as a yellow oil. LCMS (ESI) calc'd for C28H38N4O3S+ [M+H]+ m/z 511.27, found 511.

### Step 3 Synthesis of tert-butyl (S)-(1-(5-(5-(1-isopropylpiperidin-4-yl)pyridin-2-yl)-3-methylthiophene-2-carbonyl)pyrrolidin-3-yl)carbamate

To a solution of tert-butyl (S)-(1-(5-(1′-isopropyl-1′,2′,3′,6′-tetrahydro-[3,4′-bipyridin]-6-yl)-3-methylthiophene-2-carbonyl)pyrrolidin-3-yl)carbamate (180 mg, 0.35 mmol) in ethyl acetate (10 mL) was added PtO₂ (8 mg, 0.035 mmol), then the mixture was stirred at rt under H₂ for 16 h. The LCMS showed the reaction was completed and the desired mass was detected. The resulting mixture was filtered and concentrated under reduced pressure to afford tert-butyl (S)-(1-(5-(5-(1-isopropylpiperidin-4-yl)pyridin-2-yl)-3-methylthiophene-2-carbonyl)pyrrolidin-3-yl)carbamate (75 mg, 39.4% yield) as a yellow solid. LCMS (ESI) calc'd for C28H40N4O3S+ [M+H]+ m/z 513.28, found 513.

### Step 4 Synthesis of (S)-(3-aminopyrrolidin-1-yl)(5-(5-(1-isopropylpiperidin-4-yl)pyridin-2-yl)-3-methylthiophen-2-yl)methanone

A solution of tert-butyl (S)-(1-(5-(5-(1-isopropylpiperidin-4-yl)pyridin-2-yl)-3-methylthiophene-2-carbonyl)pyrrolidin-3-yl)carbamate (75 mg, 0.15 mmol) in HCl (2M in EtOAc, 5 mL) was stirred at rt for 16 h. The LCMS showed the reaction was completed and the desired mass was detected. The mixture was concentrated under reduced pressure to give the residue, which was purified by prep-HPLC to afford (S)-(3-aminopyrrolidin-1-yl)(5-(5-(1-isopropylpiperidin-4-yl)pyridin-2-yl)-3-methylthiophen-2-yl)methanone (Compound 127, 30.6 mg, 50.2% yield) as a yellow solid. LCMS (ESI) calc'd for C23H32N4OS+ [M+H]+ m/z 413.23, found 413. ¹H NMR (400 MHz, MeOD) δ 8.58 (s, 1H), 8.34 (s, 1H), 8.13 (s, 1H), 7.74 (s, 1H), 3.93 (s, 2H), 3.75 (s, 1H), 3.64 (s, 2H), 3.52 (s, 3H), 3.18 (s, 2H), 2.35 (d, J=23.5 Hz, 4H), 2.24-2.02 (m, 5H), 1.35 (d, J=5.8 Hz, 6H).

The following compound was prepared analogously to Compound 127:

| Compound | Name | H NMR | MS |
|---|---|---|---|
| 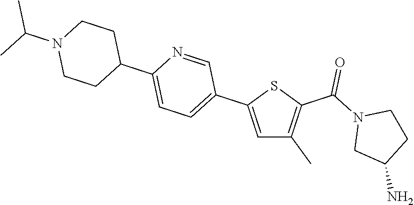 [SMILES: Cc1cc(-c2ccc(C3CCN(C(C)C)CC3)nc2)sc1C(=O)N1CC[C@H](N)C1] | (S)-(3- aminopyrrolidin- 1-yl)(5-(6-(1- isopropylpiperidin- 4-yl)pyridin-3- yl)-3- methylthiophen-2- yl)methanone | ¹H NMR (400 MHz, MeOD) δ 9.00 (s, 1H), 8.70 (d, J = 7.2 Hz, 1H), 7.99 (d, J = 7.6 Hz, 1H), 7.57 (s, 1H), 3.92 (d, J = 11.1 Hz, 2H), 3.74 (s, 1H), 3.70-3.50 (m, 5H), 3.44 (s, 1H), 2.37 (s, 1H), 2.30 (s, 7H), 2.13-2.02 (m, 1H), 1.36 (d, J = 6.5 Hz, 6H). | 413.1 [M + H]⁺ |
| 126 | | | |

### Example 71: Synthesis of (S)-(3-aminopyrrolidin-1-yl)(5-(2-(1-isopropylpiperidin-4-yl)pyrimidin-5-yl)-3-methylthiophen-2-yl)methanone (Compound 125)

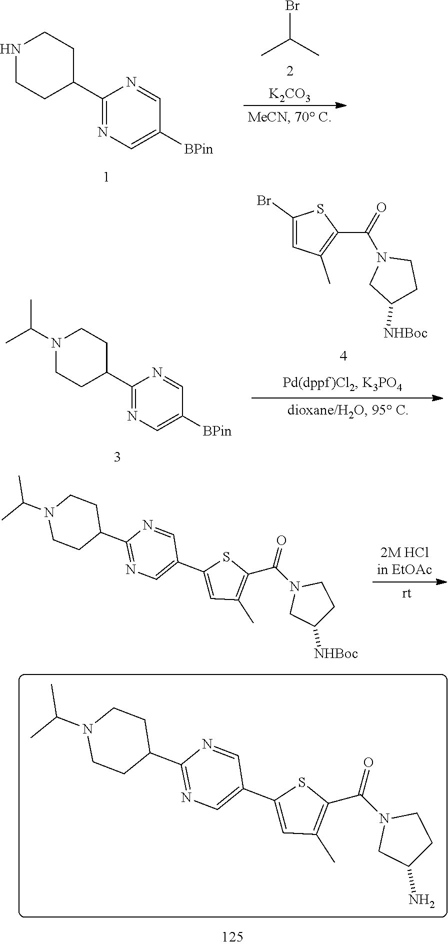

### Step 1 Synthesis of 2-(1-isopropylpiperidin-4-yl)-5-(4,4,5,5-tetramethyl-1,3,2-dioxaborolan-2-yl)pyrimidine

To a solution of 2-(piperidin-4-yl)-5-(4,4,5,5-tetramethyl-1,3,2-dioxaborolan-2-yl)pyrimidine (100 mg, 0.35 mmol) in MeCN (3 mL) was added 2-bromopropane (85 mg, 0.69 mmol) and potassium carbonate (96 mg, 0.69 mmol), then the mixture was stirred at 70° C. for 12 hours. LCMS showed the reaction was completed and the desired mass was detected. The reaction mixture was concentrated to dryness to afford 2-(1-isopropylpiperidin-4-yl)-5-(4,4,5,5-tetramethyl-1,3,2-dioxaborolan-2-yl)pyrimidine (92 mg, 64.3% yield) as a white solid. LCMS (ESI) calc'd for C18H30BN3O2+ [M+H]+ m/z 332.24, found 332.2.

### Step 2 Synthesis of tert-butyl (S)-(1-(5-(2-(1-isopropylpiperidin-4-yl)pyrimidin-5-yl)-3-methylthiophene-2-carbonyl)pyrrolidin-3-yl)carbamate

To a solution of 2-(1-isopropylpiperidin-4-yl)-5-(4,4,5,5-tetramethyl-1,3,2-dioxaborolan-2-yl)pyrimidine (92 mg, 0.28 mmol) in 1,4-dioxane (4 mL) and H₂O (0.5 mL) was added tert-butyl (S)-(1-(5-bromo-3-methylthiophene-2-carbonyl)pyrrolidin-3-yl)carbamate (97 mg, 0.25 mmol), K₃PO₄ (177 mg, 0.83 mmol) and Pd(dppf)Cl₂·DCM (34 mg, 0.04 mmol), then it was stirred at 95° C. for 16 hours. LCMS showed the reaction was completed and the desired mass was detected. The reaction mixture was concentrated to dryness to give the crude product, which was purified by column chromatography on silica gel (methanol:dichloromethane=0%~30%) to afford tert-butyl (S)-(1-(5-(2-(1-isopropylpiperidin-4-yl)pyrimidin-5-yl)-3-methylthiophene-2-carbonyl)pyrrolidin-3-yl)carbamate (77 mg, 48.6% yield) as a black solid.

MS (ESI): mass calc'd. for C27H39N503S+ [M+H]+ m/z 514.28, found 514.3.

### Step 3 Synthesis of (S)-(3-aminopyrrolidin-1-yl)(5-(2-(1-isopropylpiperidin-4-yl)pyrimidin-5-yl)-3-methylthiophen-2-yl)methanone

A solution of tert-butyl (S)-(1-(5-(2-(1-isopropylpiperidin-4-yl)pyrimidin-5-yl)-3-methylthiophene-2-carbonyl)pyrrolidin-3-yl)carbamate (77 mg, 0.15 mmol) in HCl (2 M in EtOAc, 3 mL) was stirred at 25° C. for 2 hours. LCMS showed the reaction was completed and the desired mass was detected. The mixture was concentrated to dryness to give the crude product, which was purified by prep-HPLC to afford (S)-(3-aminopyrrolidin-1-yl)(5-(2-(1-isopropylpiperidin-4-yl)pyrimidin-5-yl)-3-methylthiophen-2-yl)methanone (Compound 125, 22.7 mg, 36.2% yield) as a white solid. MS (ESI): mass calc'd. for C22H31N5OS+ [M+H]+ m/z 414.22, found 414.3. ¹H NMR (400 MHz, MeOD) δ 9.02 (s, 2H), 7.47 (s, 1H), 4.06-3.96 (m, 2H), 3.88-3.74 (m, 2H), 3.73-3.49 (m, 4H), 3.30-3.17 (m, 3H), 2.52-2.36 (m, 6H), 2.27-2.05 (m, 3H), 1.42 (d, J=6.8 Hz, 6H).

### Example 72: Synthesis of ((S)-3-aminopyrrolidin-1-yl)(3-methyl-5-(4-(1-((4-methylmorpholin-2-yl)methyl)piperidin-4-yl)phenyl)thiophen-2-yl)methanone (Compound 123)

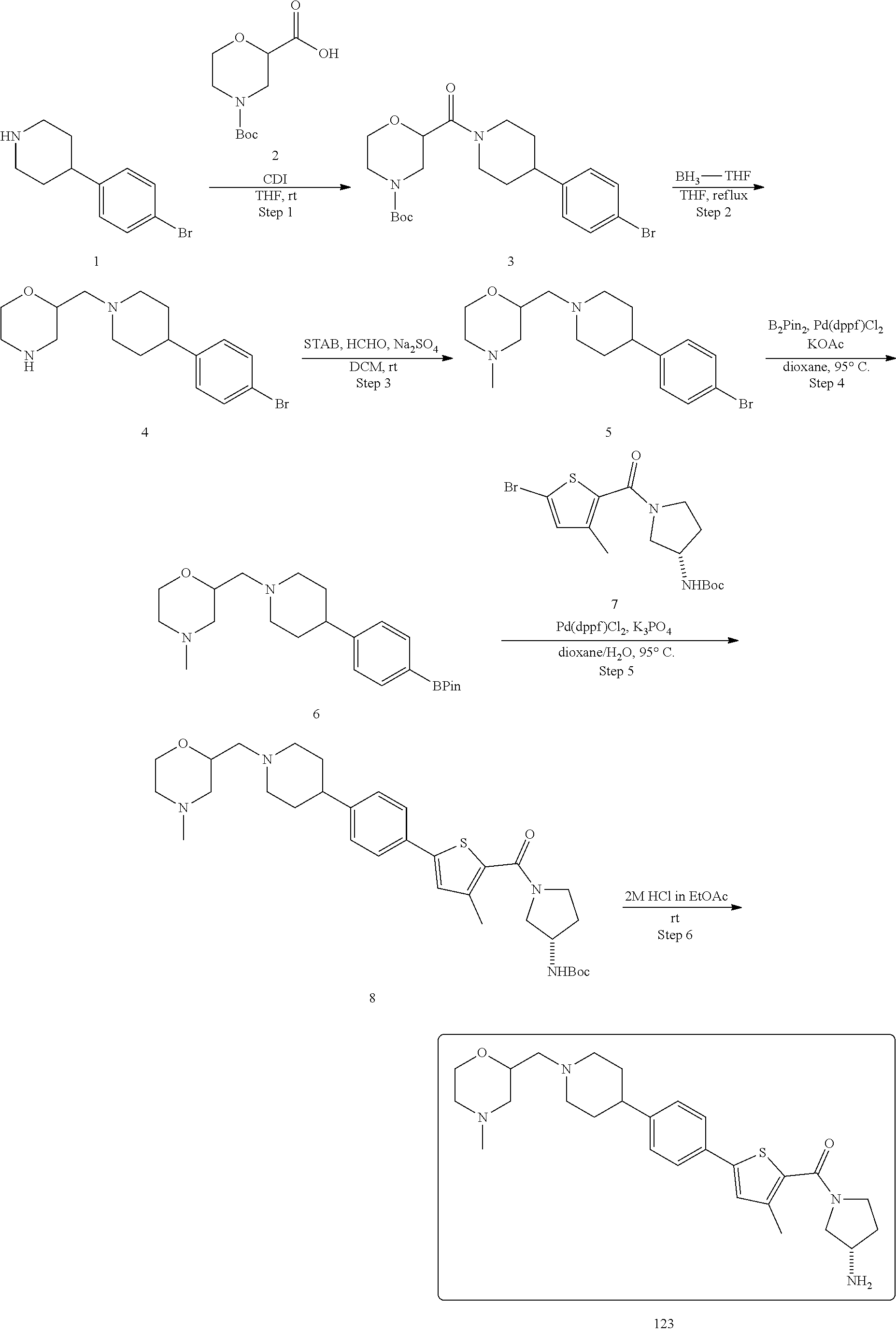

### Step 1 Synthesis of tert-butyl 2-(4-(4-bromophenyl)piperidine-1-carbonyl)morpholine-4-carboxylate

To a solution of 4-(4-bromophenyl)piperidine (500 mg, 2.08 mmol) in DMF (10 mL) was added 4-(tert-butoxycarbonyl)morpholine-2-carboxylic acid (580 mg, 2.50 mmol), DIEA (1.08 g, 8.33 mmol) and HATU (950 mg, 2.50 mmol), then the mixture was stirred at 50° C. for 12 hours. LCMS showed the reaction was completed and the desired mass was detected. The reaction mixture was diluted with H₂O (30 mL) and extracted with EtOAc (15 mL*3). The combined organic phases were washed with brine (50 mL*2), dried over Na₂SO₄, filtered and concentrated to dryness to give the crude product, which was purified by column chromatography on silica gel (dichloromethane:methanol=10:1) to afford tert-butyl 2-(4-(4-bromophenyl)piperidine-1-carbonyl)morpholine-4-carboxylate (860 mg, 81.8% yield) as yellow oil. MS (ESI): mass calc'd. for C21H29BrN2O4+ [M+H]+ m/z 453.13, found 453.1.

### Step 2 Synthesis of 2-((4-(4-bromophenyl)piperidin-1-yl)methyl)morpholine

To a mixture of tert-butyl 2-(4-(4-bromophenyl)piperidine-1-carbonyl)morpholine-4-carboxylate (860 mg, 1.89 mmol) in THF (3 ml) was added BH₃·THF (2 M in THF, 3.8 mL), then the solution was stirred at 75° C. for 4 hours under N₂ atmosphere. LCMS showed the reaction was completed and the desired mass was detected. After that, the reaction was cooled in an ice bath, and methanol (5 mL) was added cautiously. HCl (6 M in water, 8 mL) was added dropwise fast, and the mixture was heated to reflux for 30 minutes. The volatiles were then concentrated in vacuo, and the resulting mixture was cooled in an ice bath and 50% aqueous NaOH (10 mL) was added (pH>10). The mixture was then diluted with water and extracted with diethyl ether (50 mL*3). The combined organic phases were washed with brine, dried over MgSO₄, filtered and concentrated. The crude product was purified by column chromatography on silica gel (methanol:dichloromethane=0%~12%) to afford 2-((4-(4-bromophenyl)piperidin-1-yl)methyl)morpholine (600 mg, 79.4% yield) as a white solid. MS (ESI): mass calc'd. for C16H23BrN2O+ [M+H]+ m/z 339.10, found 339.1.

### Step 3 Synthesis of 2-((4-(4-bromophenyl)piperidin-1-yl)methyl)-4-methylmorpholine

To a mixture of 2-((4-(4-bromophenyl)piperidin-1-yl)methyl)morpholine (600 mg, 1.77 mmol) in DCM (60 mL) was added (CH₃COO)₃BHNa (3.37 g, 15.92 mmol), HCHO (574 mg, 7.07 mmol) and Na₂SO₄ (126 mg, 0.88 mmol), then the solution was stirred at 25° C. for 12 hours. LCMS showed the reaction was completed and the desired mass was detected. The reaction mixture was concentrated to dryness to give the crude product, which was purified by column chromatography on silica gel (dichloromethane:methanol=10:1) to afford 2-((4-(4-bromophenyl)piperidin-1-yl)methyl)-4-methylmorpholine (530 mg, 76.3% yield) as a white solid. MS (ESI): mass calc'd. for C17H25BrN2O+ [M+H]+ m/z 353.12, found 353.1.

### Step 4 Synthesis of 4-methyl-2-((4-(4-(4,4,5,5-tetramethyl-1,3,2-dioxaborolan-2-yl)phenyl)piperidin-1-yl)methyl)morpholine

To a mixture of 2-((4-(4-bromophenyl)piperidin-1-yl)methyl)-4-methylmorpholine (530 mg, 1.50 mmol) in 1,4-dioxane (13 mL) was added B₂pin₂ (457 mg, 1.80 mmol), potassium acetate (442 mg, 4.50 mmol) and Pd(pddf)Cl₂·DCM (184 mg, 0.23 mmol), then the suspension was stirred at 100° C. for 16 hours under N₂ atmosphere. LCMS showed the reaction was completed and the desired mass was detected. The reaction mixture was concentrated to dryness to give the crude product, which was purified by column chromatography on silica gel (methanol:dichloromethane=0%~30%) to afford 4-methyl-2-((4-(4-(4,4,5,5-tetramethyl-1,3,2-dioxaborolan-2-yl)phenyl)piperidin-1-yl)methyl)morpholine (530 mg, 79.4%) as a black solid.

MS (ESI): mass calc'd. for C23H37BN2O3+ [M+H]⁺ m/z 401.29, found 401.1.

### Step 5 Synthesis of tert-butyl ((3S)-1-(3-methyl-5-(4-(1-((4-methylmorpholin-2-yl)methyl)piperidin-4-yl)phenyl)thiophene-2-carbonyl)pyrrolidin-3-yl)carbamate

To a mixture of 4-methyl-2-((4-(4-(4,4,5,5-tetramethyl-1,3,2-dioxaborolan-2-yl)phenyl)piperidin-1-yl)methyl)morpholine (530 mg, 1.32 mmol) in 1,4-dioxane (15 mL) and H₂O (2 mL) was added compound 7 (464 mg, 1.19 mmol), K₃PO₄ (843 mg, 3.97 mmol) and Pd(dppf)Cl₂·DCM (162 mg, 0.20 mmol), then the mixture was stirred at 95° C. for 16 hours. LCMS showed the reaction was completed and the desired mass was detected. The reaction mixture was concentrated to dryness to give the crude product, which was purified by column chromatography on silica gel (methanol:ethyl acetate=10%-20%) to afford tert-butyl ((3S)-1-(3-methyl-5-(4-(1-((4-methylmorpholin-2-yl)methyl)piperidin-4-yl)phenyl)thiophene-2-carbonyl)pyrrolidin-3-yl)carbamate (400 mg, 46.7%) as a green solid. MS (ESI): mass calc'd. for C32H46N4O4S+ [M+H]+ m/z 583.32, found 583.3.

### Step 6 Synthesis of ((S)-3-aminopyrrolidin-1-yl)(3-methyl-5-(4-(1-((4-methylmorpholin-2-yl)methyl)piperidin-4-yl)phenyl)thiophen-2-yl)methanone

A mixture of tert-butyl ((3S)-1-(3-methyl-5-(4-(1-((4-methylmorpholin-2-yl)methyl)piperidin-4-yl)phenyl)thiophene-2-carbonyl)pyrrolidin-3-yl)carbamate (200 mg, 0.34 mmol) in HCl (2 M in EtOAc, 2 mL) was stirred at 25° C. for 3 hours. LCMS showed the reaction was completed and the desired mass was detected. The reaction mixture was concentrated to dryness to give the crude product, which was purified by prep-HPLC to afford ((S)-3-aminopyrrolidin-1-yl)(3-methyl-5-(4-(1-((4-methylmorpholin-2-yl)methyl)piperidin-4-yl)phenyl)thiophen-2-yl)methanone (Compound 123, 30.4 mg, 18.2%) as a white solid. MS (ESI): mass calc'd. for C₂₇H₃₈N₄O₂S+ [M+H]+ m/z 483.27, found 483.4. ¹H NMR (400 MHz, MeOD) δ 7.62 (d, J=8.4 Hz, 2H), 7.33 (d, J=8.0 Hz, 2H), 7.25 (s, 1H), 4.39 (s, 1H), 4.21 (dd, J=12.8, 3.2 Hz, 1H), 4.02-3.93 (m, 3H), 3.86-3.68 (m, 5H), 3.52 (t, J=12.4 Hz, 2H), 3.36 (d, J=7.6 Hz, 2H), 3.29-3.12 (m, 3H), 3.01-2.92 (m, 5H), 2.49-2.39 (m, 1H), 2.35 (s, 3H), 2.20-2.05 (m, 5H).

### Example 73: Synthesis of (S)-(3-aminopyrrolidin-1-yl)(5-(2-chloro-4-(piperidin-4-yl)phenyl)-3-methylthiophen-2-yl)methanone (Compound 122)

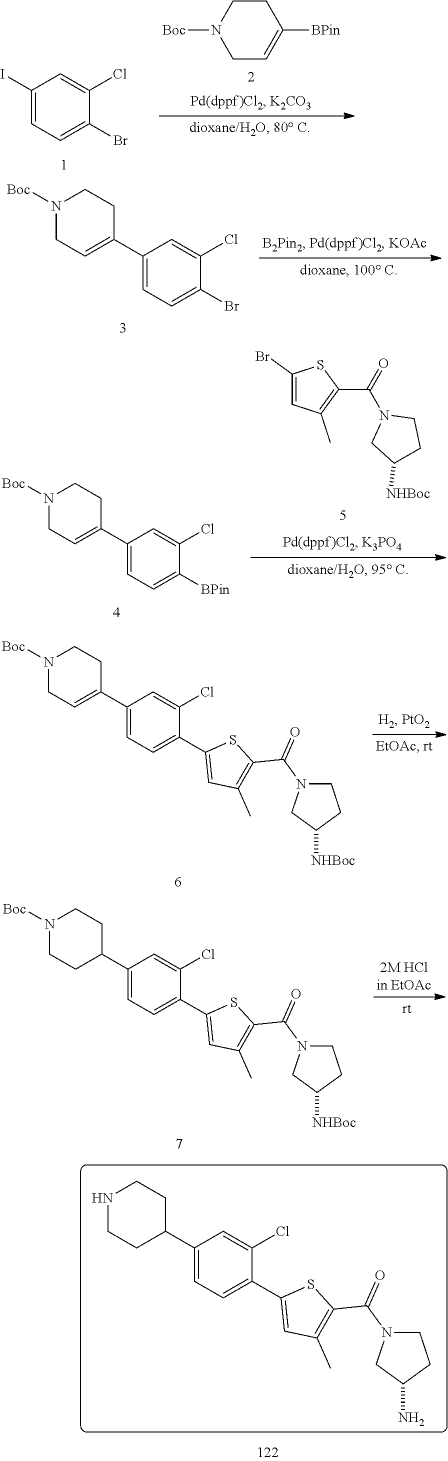

### Step 1 Synthesis of tert-butyl 4-(4-bromo-3-chlorophenyl)-3,6-dihydropyridine-1(2H)-carboxylate

To a mixture of tert-butyl [4-(4,4,5,5-tetramethyl-1,3,2-dioxaborolan-2-yl)-3,6-dihydro-2H-pyridin-1-yl]formate (1.20 g, 3.90 mmol) in 1,4-dioxane (24 mL) and H₂O (3 mL) was added 1-bromo-2-fluoro-4-iodobenzene (1.86 g, 5.80 mmol), potassium carbonate (1.35 g, 9.70 mmol) and Pd(dppf)Cl₂·DCM (0.48 g, 0.50 mmol), then the mixture was stirred at 80° C. for 16 hours under N₂ atmosphere. LCMS showed the reaction was completed and the desired mass was detected. The reaction mixture was concentrated to dryness to give the crude product, which was purified by column chromatography on silica gel (ethyl acetate:petroleum ether=4%) to afford tert-butyl 4-(4-bromo-3-chlorophenyl)-3,6-dihydropyridine-1(2H)-carboxylate (1.00 g, 64.1% yield) as light green oil. MS (ESI): mass calc'd. for C16H19BrClNO2+ [M+H]⁺ m/z 372.03, found 372.0.

### Step 2 Synthesis of tert-butyl 4-(3-chloro-4-(4,4,5,5-tetramethyl-1,3,2-dioxaborolan-2-yl)phenyl)-3,6-dihydropyridine-1(2H)-carboxylate

To a solution of tert-butyl 4-(4-bromo-3-chlorophenyl)-3,6-dihydropyridine-1(2H)-carboxylate (1.00 g, 2.70 mmol) in 1,4-dioxane (20 mL) was added B₂pin₂ (0.82 g, 3.20 mmol), potassium acetate (0.79 g, 8.10 mmol) and Pd(dppf)Cl₂ (0.33 g, 0.40 mmol), then the mixture was stirred at 100° C. for 16 hours under N₂ atmosphere. LCMS showed the reaction was completed and the desired mass was detected. The reaction mixture was concentrated to dryness to give the crude product, which was purified by column chromatography on silica gel (ethyl acetate:petroleum ether=4%) to afford tert-butyl 4-(3-chloro-4-(4,4,5,5-tetramethyl-1,3,2-dioxaborolan-2-yl)phenyl)-3,6-dihydropyridine-1(2H)-carboxylate (0.89 g, 70.4% yield) as a white solid. MS (ESI): mass calc'd. for C22H31BClNO4+ [M+H]⁺ m/z 420.20, found 420.0.

### Step 3 Synthesis of tert-butyl (S)-4-(4-(5-(3-((tert-butoxycarbonyl)amino)pyrrolidine-1-carbonyl)-4-methylthiophen-2-yl)-3-chlorophenyl)-3,6-dihydropyridine-1(2H)-carboxylate

To a solution of tert-butyl 4-(3-chloro-4-(4,4,5,5-tetramethyl-1,3,2-dioxaborolan-2-yl)phenyl)-3,6-dihydropyridine-1(2H)-carboxylate (290 mg, 0.69 mmol) in 1,4-dioxane (8 mL) and H₂O (1 mL) was added compound 5 (241 mg, 0.62 mmol), K₃PO₄ (439 mg, 2.07 mmol) and Pd(dppf)Cl₂·DCM (84 mg, 0.10 mmol), then the mixture was stirred at 95° C. for 16 hours. LCMS showed the reaction was completed and the desired mass was detected. The reaction mixture was concentrated to dryness to give the crude product, which was purified by column chromatography on silica gel (ethyl acetate:petroleum ether=35%) to afford tert-butyl (S)-4-(4-(5-(3-((tert-butoxycarbonyl)amino)pyrrolidine-1-carbonyl)-4-methylthiophen-2-yl)-3-chlorophenyl)-3,6-dihydropyridine-1(2H)-carboxylate (480 mg, 57.7% yield) as a black solid.

MS (ESI): mass calc'd. for C31H40ClN3O5S+ [M+H]⁺ m/z 602.24, found 602.0.

### Step 4 Synthesis of tert-butyl (S)-4-(4-(5-(3-((tert-butoxycarbonyl)amino)pyrrolidine-1-carbonyl)-4-methylthiophen-2-yl)-3-chlorophenyl)piperidine-1-carboxylate

To a mixture of tert-butyl (S)-4-(4-(5-(3-((tert-butoxycarbonyl)amino)pyrrolidine-1-carbonyl)-4-methylthiophen-2-yl)-3-chlorophenyl)-3,6-dihydropyridine-1(2H)-carboxylate (480 mg, 0.80 mmol) in EtOAc (4 mL) was added PtO₂ (49 mg, 0.21 mmol), then the suspension was stirred at 25° C. for 32 hours under H₂ atmosphere. LCMS showed the reaction was completed and the desired mass was detected. The reaction mixture was concentrated to dryness to give the crude product, which was purified by column chromatography on silica gel (ethyl acetate:petroleum ether=35%) to afford tert-butyl (S)-4-(4-(5-(3-((tert-butoxycarbonyl)amino)pyrrolidine-1-carbonyl)-4-methylthiophen-2-yl)-3-chlorophenyl)piperidine-1-carboxylate (200 mg, 37.4%) as colorless oil.

MS (ESI): mass calc'd. for C31H42ClN₃O5S+ [M+H]⁺ m/z 604.25, found 604.0.

### Step 5 Synthesis of (S)-(3-aminopyrrolidin-1-yl)(5-(2-chloro-4-(piperidin-4-yl)phenyl)-3-methylthiophen-2-yl)methanone

A mixture of tert-butyl (S)-4-(4-(5-(3-((tert-butoxycarbonyl)amino)pyrrolidine-1-carbonyl)-4-methylthiophen-2-yl)-3-chlorophenyl)piperidine-1-carboxylate (200 mg, 0.33 mmol) in HCl (2 M in EtOAc, 2 mL) was stirred at 25° C. for 3 hours. LCMS showed the reaction was completed and the desired mass was detected. The reaction mixture was concentrated to dryness to give the crude product, which was purified by prep-HPLC to afford (S)-(3-aminopyrrolidin-1-yl)(5-(2-chloro-4-(piperidin-4-yl)phenyl)-3-methylthiophen-2-yl)methanone (Compound 122, 22.7 mg, 16.5%) as a white solid. MS (ESI): mass calc'd. for C21H26ClN3OS+ [M+H]⁺ m/z 404.15, found 404.2. ¹H NMR (400 MHz, MeOD) δ 7.58 (d, J=8.0 Hz, 1H), 7.46 (d, J=1.6 Hz, 1H), 7.30 (dd, J=8.0, 1.6 Hz, 1H), 7.22 (s, 1H), 4.03-3.97 (m, 2H), 3.87-3.75 (m, 2H), 3.73-3.66 (m, 1H), 3.57-3.49 (m, 2H), 3.22-3.10 (m, 2H), 3.03-2.92 (m, 1H), 2.50-2.40 (m, 1H), 2.38-2.36 (m, 3H), 2.18-2.06 (m, 3H), 1.98-1.85 (m, 2H).

The following compounds were prepared analogously to Compound 122:

| Compound | Name | H NMR | MS |
|---|---|---|---|
| 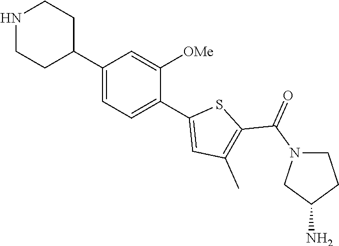 [SMILES: COc1cc(C2CCNCC2)ccc1-c1cc(C)c(C(=O)N2CC[C@H](N)C2)s1] | (S)-(3- aminopyrrolidin- 1-yl)(5-(2- methoxy-4- (piperidin-4- yl)phenyl)-3- methylthiophen- 2-yl)methanone | ¹H NMR (400 MHz, MeOD) δ 8.57 (s, 2H), 7.68 (d, J = 8.0 Hz, 1H), 7.37 (s, 1H), 7.01 (s, 1H), 6.94 (dd, J = 8.1, 1.3 Hz, 1H), 3.99 (s, 1H), 3.97 (s, 3H), 3.94 (s, 1H), 3.89-3.80 (m, 2H), 3.74 (ddd, J = 10.8, 7.6, 5.5 Hz, 1H), 3.65 (d, J = 8.4 Hz, 1H), 3.52 (d, J = 12.7 Hz, 2H), 3.14 (td, J = 12.8, 2.8 Hz, 2H), 2.93 (ddd, J = 11.9, 7.7, 3.4 Hz, 1H), 2.41 (dd, J = 13.5, 7.0 Hz, 1H), 2.36 (s, 3H), 2.14-2.06 (m, 3H), 2.05 (s, 1H), 2.02-1.93 (m, 2H). | 400.2 [M + H]⁺ |
| 121 | | | |
| 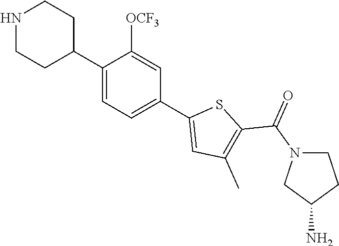 [SMILES: Cc1cc(-c2ccc(C3CCNCC3)c(OC(F)(F)F)c2)sc1C(=O)N1CC[C@H](N)C1] | (S)-(3- aminopyrrolidin- 1-yl)(3-methyl-5- (4-(piperidin-4- yl)-3- (trifluoromethoxy) phenyl)thiophen- 2-yl)methanone | ¹H NMR (400 MHz, MeOD) δ 7.67 (dd, J = 8.4, 2.0 Hz, 1H), 7.56-7.53 (m, 1H), 7.49 (d, J = 8.0 Hz, 1H), 7.33 (s, 1H), 4.03- 3.95 (m, 2H), 3.89-3.81 (m, 1H), 3.80-3.73 (m, 1H), 3.73- 3.66 (m, 1H), 3.57-3.50 (m, 2H), 3.36-3.32 (m, 0.5H), 3.30- 3.26 (m, 0.5H), 3.20 (td, J = 12.8, 4.0 Hz, 2H), 2.50-2.40 (m, 1H), 2.36 (s, 3H), 2.18-2.10 (m, 1H), 2.06-1.92 (m, 4H). | 454.2 [M + H]⁺ |
| 116 | | | |
| | (S)-(3- | ¹H NMR (400 MHz, MeOD) δ | 454.3 |
| | aminopyrrolidin- | 7.77 (d, J = 8.4 Hz, 1H), 7.40- | [M + H]⁺ |
| | 1-yl)(3-methyl-5- | 7.35 (m, 1H), 7.35-7.32 (m, | |
| | (4-(piperidin-4- | 1H), 7.31 (s, 1H), 4.04-3.95 (m, | |
| | yl)-2- | 2H), 3.88-3.74 (m, 2H), 3.73- | |
| | (trifluoromethoxy) | 3.65 (m, 1H), 3.56-3.49 (m, | |
| | phenyl)thiophen- | 2H), 3.21-3.12 (m, 2H), 3.07- | |
| | 2-yl)methanone | 2.98 (m, 1H), 2.50-2.40 (m, | |
| | | 1H), 2.37 (s, 3H), 2.18-2.09 (m, | |
| | | 3H), 1.99-1.87 (m, 2H). | |

The following compounds are prepared analogously to the methods described in this example:

| Compound | Synthesis |
|---|---|
| 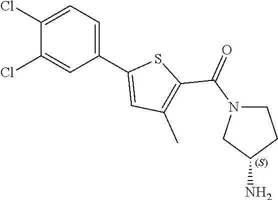 | For step 2, a corresponding aryl bromide can be used to synthesize compound 244 |
| 244 | |
| 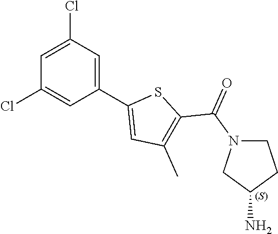 | For step 2, a corresponding aryl bromide can be used to synthesize compound 245 |
| 245 | |

### Example 74: Synthesis of (S)-(3-aminopyrrolidin-1-yl)(5-(2-hydroxy-4-(piperidin-4-yl)phenyl)-3-methylthiophen-2-yl)methanone (Compound 115)

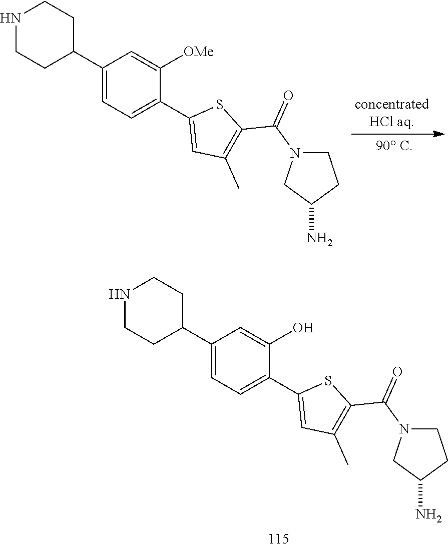 [SMILES: COc1cc(C2CCNCC2)ccc1-c1cc(C)c(C(=O)N2CC[C@H](N)C2)s1 ; Cc1cc(-c2ccc(C3CCNCC3)cc2O)sc1C(=O)N1CC[C@H](N)C1]

### Step 1 Synthesis of (S)-(3-aminopyrrolidin-1-yl)(5-(2-hydroxy-4-(piperidin-4-yl)phenyl)-3-methylthiophen-2-yl)methanone

A solution of (S)-(3-aminopyrrolidin-1-yl)(5-(2-methoxy-4-(piperidin-4-yl)phenyl)-3-methylthiophen-2-yl)methanone (100 mg, 0.24 mmol) in concentrated HCl (5 mL) was stirred at 90° C. for 16 h. The LCMS showed the reaction was completed and the desired mass was detected. After cooling to rt, the resulted mixture was concentrated under reduced pressure to give the residue, which was purified by prep-HPLC to afford (S)-(3-aminopyrrolidin-1-yl)(5-(2-hydroxy-4-(piperidin-4-yl)phenyl)-3-methylthiophen-2-yl)methanone (Compound 115, 16.28 mg, 16.7% yield) as a yellow solid. LCMS (ESI) calc'd for C22H29N3O2S+ [M+H]+ m/z 386.18, found 386. H NMR (400 MHz, MeOD) δ 7.49 (d, J=8.1 Hz, 1H), 7.28 (s, 1H), 6.74 (d, J=1.6 Hz, 1H), 6.70 (dd, J=8.1, 1.6 Hz, 1H), 3.92-3.85 (m, 2H), 3.80-3.56 (m, 5H), 3.38 (t, J=9.7 Hz, 2H), 3.02 (td, J=12.8, 2.7 Hz, 2H), 2.34 (dt, J=13.6, 6.6 Hz, 1H), 2.27-2.19 (m, 4H), 2.10-1.90 (m, 4H), 1.84 (dd, J=13.0, 3.8 Hz, 1H), 1.77 (dd, J=13.5, 3.2 Hz, 1H), 0.83 (ddd, J=13.0, 7.1, 4.8 Hz, 1H).

The following compounds were prepared analogously to Compound 115:

| Compound | Name | H NMR | MS |
|---|---|---|---|
| 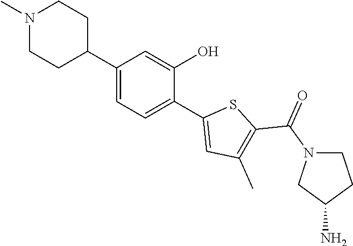 [SMILES: Cc1cc(-c2ccc(C3CCN(C)CC3)cc2O)sc1C(=O)N1CC[C@H](N)C1] | (S)-(3- aminopyrrolidin-1- yl)(5-(2-hydroxy- 4-(1- methylpiperidin-4- yl)phenyl)-3- methylthiophen-2- yl)methanone | ¹H NMR (400 MHz, MeOD) δ 7.49 (d, J = 8.1 Hz, 1H), 7.28 (s, 1H), 6.74 (s, 1H), 6.70 (d, J = 8.0 Hz, 1H), 3.89 (d, J = 8.2 Hz, 2H), 3.78-3.65 (m, 2H), 3.60 (dd, J = 15.2, 6.8 Hz, 1H), 3.50 (d, J = 11.0 Hz, 2H), 3.03 (t, J = 11.5 Hz, 2H), 2.81 (s, 3H), 2.72 (t, J = 11.6 Hz, 1H), 2.33 (td, J = 13.9, 7.5 Hz, 1H), 2.25 (s, 3H), 2.06-1.96 (m, 3H), 1.93-1.82 (m, 2H). | 400.00 [M + H]⁺ |
| 114 | | | |
| 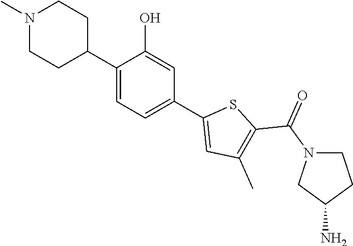 | (S)-(3- aminopyrrolidin-1- yl)(5-(3-hydroxy- 4-(piperidin-4- yl)phenyl)-3- methylthiophen-2- yl)methanone | ¹H NMR (400 MHz, MeOD) δ 7.16 (t, J = 4.0 Hz, 1H), 7.10 (dd, J = 7.9, 1.8 Hz, 1H), 7.06 (d, J = 1.7 Hz, 1H), 4.02-3.95 (m, 1H), 3.84 (dt, J = 11.7, 7.5 Hz, 1H), 3.78-3.64 (m, 1H), 3.50 (d, J = 12.5 Hz, 1H), 3.26- 3.09 (m, 2H), 2.45 (tt, J = 14.0, 6.9 Hz, 1H), 2.34 (s, 2H), 2.14- 1.95 (m, 3H). | 386.00 [M + H]⁺ |
| 107 | | | |

### Example 75: Synthesis of (S)-(3-aminopyrrolidin-1-yl)(5-(3-hydroxy-4-(1-methylpiperidin-4-yl)phenyl)-3-methylthiophen-2-yl)methanone (Compound 106)

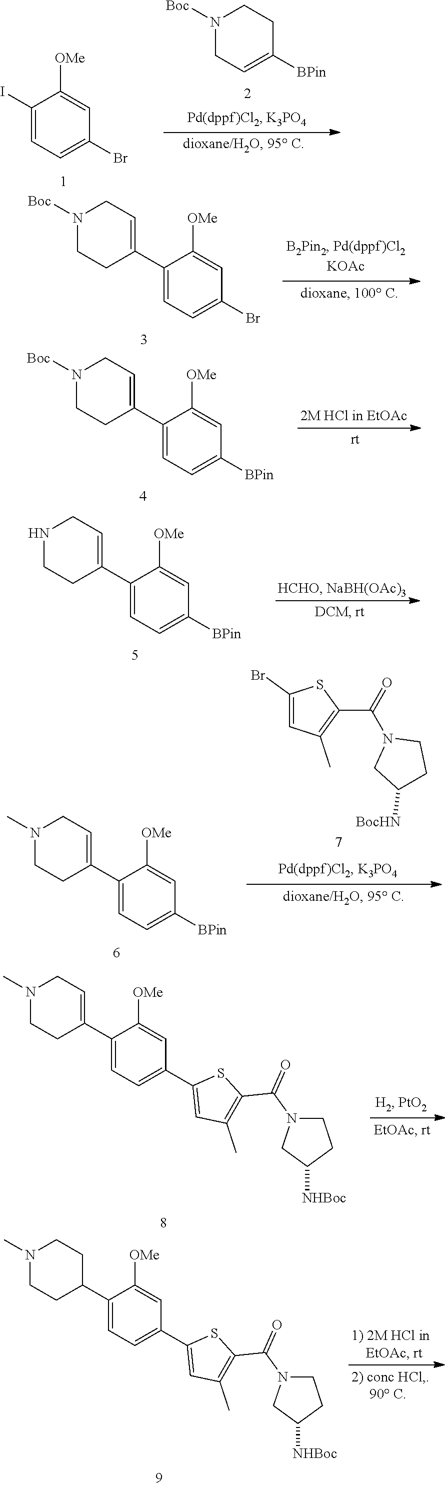

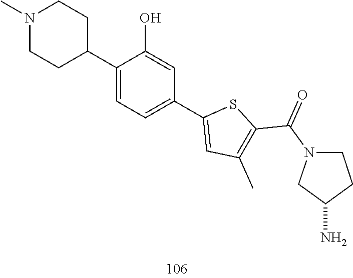 [SMILES: Cc1cc(-c2ccc(C3CCN(C)CC3)c(O)c2)sc1C(=O)N1CC[C@H](N)C1]

### Step 1 Synthesis of tert-butyl 4-(4-bromo-2-methoxyphenyl)-3,6-dihydropyridine-1(2H)-carboxylate

To a solution of 4-bromo-1-iodo-2-methoxybenzene (2.0 g, 6.4 mmol) in dioxane/H₂O (v:v=8:1, 45 mL) was added tert-butyl [4-(4,4,5,5-tetramethyl-1,3,2-dioxaborolan-2-yl)-3,6-dihydro-2H-pyridin-1-yl]formate (1.98 g, 6.4 mmol), K₃PO₄ (4.07 g, 19.2 mmol) and Pd(dppf)Cl₂ (1.04 g, 1.28 mmol), then the mixture was stirred at 95° C. under N₂ for 16 h. After cooling to rt, the mixture was filtered and concentrated under reduced pressure to give the residue, which was purified by flash chromatography (petroleum:ether ethyl acetate=10:1) to afford [4-(4-bromo-2-methoxyphenyl)-3,6-dihydro-2H-pyridin-1-yl]tert-butyl formate (1.20 g, 48.3% yield) as yellow oil. LCMS (ESI) calc'd for C17H22BrNO3+ [M+H]+ m/z 368.08, found 368.

### Step 2 Synthesis of tert-butyl 4-(2-methoxy-4-(4,4,5,5-tetramethyl-1,3,2-dioxaborolan-2-yl)phenyl)-3,6-dihydropyridine-1(2H)-carboxylate

To a solution of tert-butyl 4-(4-bromo-2-methoxyphenyl)-3,6-dihydropyridine-1(2H)-carboxylate (1.20 g, 3.25 mmol) in dioxane (20 mL) was added 4,4,5,5-tetramethyl-2-(4,4,5,5-tetramethyl-1,3,2-dioxaborolan-2-yl)-1,3,2-dioxaborolane (825.2 mg, 3.25 mmol), potassium acetate (956.8 mg, 9.75 mmol) and Pd(dppf)Cl₂ (530.3 mg, 0.65 mmol), then the mixture was stirred at 95° C. under N₂ for 16 h. After cooling to rt, the mixture was filtered and concentrated under reduced pressure to give the residue, which was purified by flash chromatography (petroleum:ether ethyl acetate=10:1) to afford tert-butyl 4-(2-methoxy-4-(4,4,5,5-tetramethyl-1,3,2-dioxaborolan-2-yl)phenyl)-3,6-dihydropyridine-1(2H)-carboxylate (1.00 g, 70.2% yield) as yellow oil. LCMS (ESI) calc'd for C23H34BNO5+ [M+H]+ m/z 416.25, found 416.

### Step 3 Synthesis of tert-butyl 4-(2-methoxy-4-(4,4,5,5-tetramethyl-1,3,2-dioxaborolan-2-yl)phenyl)-1,2,3,6-tetrahydropyridine

A solution of tert-butyl {4-[2-methoxy-4-(4,4,5,5-tetramethyl-1,3,2-dioxaborolan-2-yl)phenyl]-3,6-dihydro-2H-pyridin-1-yl}formate (580 mg, 1.4 mmol) in HCl (2M in EA, 5 mL) was stirred at rt for 16 h. The LCMS showed the reaction was completed and the desired mass was detected. The resulting mixture was concentrated under reduced pressure to afford 4-(2-methoxy-4-(4,4,5,5-tetramethyl-1,3,2-dioxaborolan-2-yl)phenyl)-1,2,3,6-tetrahydropyridine (509 mg, 110% yield) as yellow oil. LCMS (ESI) calc'd for C18H26BNO3+ [M+H]+ m/z 316.2, found 316.

### Step 4 Synthesis of 4-(2-methoxy-4-(4,4,5,5-tetramethyl-1,3,2-dioxaborolan-2-yl)phenyl)-1-methyl-1,2,3,6-tetrahydropyridine

To a solution of 4-(2-methoxy-4-(4,4,5,5-tetramethyl-1,3,2-dioxaborolan-2-yl)phenyl)-1,2,3,6-tetrahydropyridine (1.40 g, 4.44 mmol) in DCM (5 mL) was added HCHO (500 mg, 12 mmol) and sodium triacetoxyborohydride (5.60 g, 12 mmol), then the mixture was stirred at rt for 16 h. The LCMS showed the reaction was completed and the desired mass was detected. The mixture was concentrated under reduced pressure to give the residue, which was purified by flash chromatography (methanol:ethyl acetate=1:10) to afford 4-(2-methoxy-4-(4,4,5,5-tetramethyl-1,3,2-dioxaborolan-2-yl)phenyl)-1-methyl-1,2,3,6-tetrahydropyridine (613 mg, 39.8% yield) as yellow oil. LCMS (ESI) calc'd for C19H28BNO3+ [M+H]+ m/z 330.22, found 330.

### Step 5 Synthesis of tert-butyl (S)-(1-(5-(3-methoxy-4-(1-methyl-1,2,3,6-tetrahydropyridin-4-yl)phenyl)-3-methylthiophene-2-carbonyl)pyrrolidin-3-yl)carbamate

To a solution of 4-(2-methoxy-4-(4,4,5,5-tetramethyl-1,3,2-dioxaborolan-2-yl)phenyl)-1-methyl-1,2,3,6-tetrahydropyridine (380 mg, 0.98 mmol) in dioxane/H₂O (v:v=8:1, 18 mL) was added 4-[2-methoxy-4-(4,4,5,5-tetramethyl-1,3,2-dioxaborolan-2-yl)phenyl]-1-methyl-3,6-dihydro-2H-pyridine (321.4 mg, 0.98 mmol), K₃PO₄ (621.6 mg, 2.93 mmol) and Pd(dppf)Cl₂ (159.3 mg, 0.19 mmol), then the mixture was stirred at 95° C. under N₂ for 16 h. After cooling to rt, the mixture was filtered and concentrated under reduced pressure to give the residue was purified by flash chromatography (methanol:ethyl acetate=1:5) to afford tert-butyl (S)-(1-(5-(3-methoxy-4-(1-methyl-1,2,3,6-tetrahydropyridin-4-yl)phenyl)-3-methylthiophene-2-carbonyl)pyrrolidin-3-yl)carbamate (220 mg, 41.85% yield) as yellow oil. LCMS (ESI) calc'd for C28H37N3O4S+ [M+H]+ m/z 512.25, found 512.

### Step 6 Synthesis of tert-butyl (S)-(1-(5-(3-methoxy-4-(1-methylpiperidin-4-yl)phenyl)-3-methylthiophene-2-carbonyl)pyrrolidin-3-yl)carbamate

A solution of tert-butyl (S)-(1-(5-(3-methoxy-4-(1-methyl-1,2,3,6-tetrahydropyridin-4-yl)phenyl)-3-methylthiophene-2-carbonyl)pyrrolidin-3-yl)carbamate (448 mg, 0.88 mmol) and PtO₂ (19.9 mg, 0.088 mmol) in ethyl acetate (20 mL) was stirred at rt under H₂ for 16 h.

The LCMS showed the reaction was completed and the desired mass was detected. The resulting mixture was filtered and concentrated under reduced pressure to afford tert-butyl (S)-(1-(5-(3-methoxy-4-(1-methylpiperidin-4-yl)phenyl)-3-methylthiophene-2-carbonyl)pyrrolidin-3-yl)carbamate (380 mg, 80.3% yield) as yellow oil. LCMS (ESI) calc'd for C28H39N3O4S+ [M+H]+ m/z 514.27, found 514.

### Step 7 Synthesis of (S)-(3-aminopyrrolidin-1-yl)(5-(3-hydroxy-4-(1-methylpiperidin-4-yl)phenyl)-3-methylthiophen-2-yl)methanone

A solution of tert-butyl (S)-(1-(5-(3-methoxy-4-(1-methylpiperidin-4-yl)phenyl)-3-methylthiophene-2-carbonyl)pyrrolidin-3-yl)carbamate (380 mg, 0.74 mmol) in HCl (2M in EtOAc, 10 mL) was stirred at rt for 16 h. The LCMS showed the reaction was completed and the desired mass was detected. The resulting mixture was concentrated under reduced pressure to afford (S)-(3-aminopyrrolidin-1-yl)(5-(3-hydroxy-4-(1-methylpiperidin-4-yl)phenyl)-3-methylthiophen-2-yl)methanone (Compound 106, 350 mg, 105.6% yield) as a yellow solid. LCMS (ESI) calc'd for C23H31N3O2S+ [M+H]+ m/z 414.21, found 414. ¹H NMR (400 MHz, MeOD) δ 7.15 (d, J=8.3 Hz, 1H), 7.10 (dd, J=7.9, 1.7 Hz, 1H), 7.07-7.05 (m, 1H), 3.98 (d, J=8.1 Hz, 1H), 3.88-3.81 (m, OH), 3.78-3.67 (m, 1H), 3.60 (d, J=10.3 Hz, 1H), 3.23-3.12 (m, 2H), 2.90 (d, J=3.4 Hz, 2H), 2.44 (td, J=13.4, 7.1 Hz, 1H), 2.34 (s, 2H), 2.11 (dd, J=15.7, 7.9 Hz, 3H).

### Example 77: Synthesis of (S)-(3-aminopyrrolidin-1-yl)(5-(4-(1-isopropylpiperidin-4-yl)phenyl)-3-(methoxymethyl)thiophen-2-yl)methanone (Compound 102)

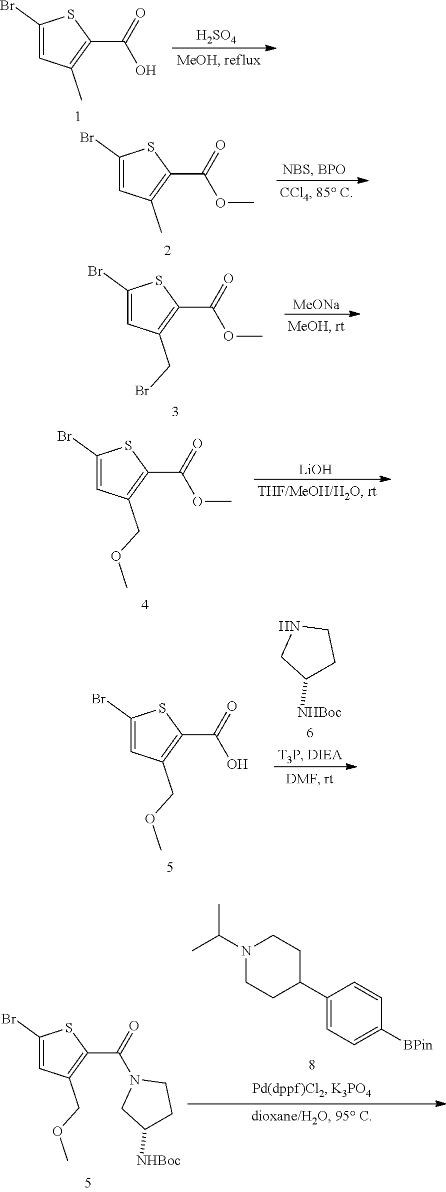

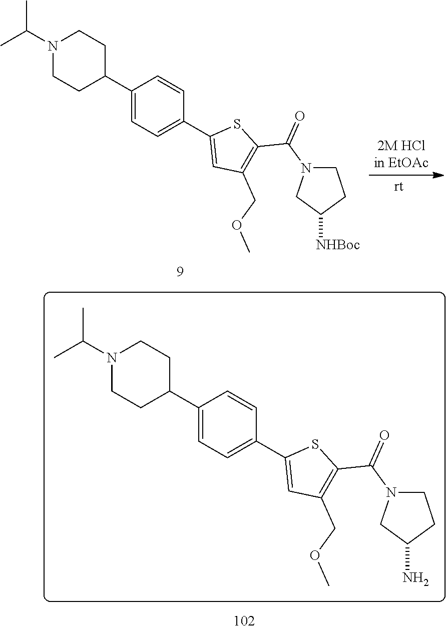 [SMILES: COCc1cc(-c2ccc(C3CCN(C(C)C)CC3)cc2)sc1C(=O)N1CC[C@H](NC(=O)OC(C)(C)C)C1 ; COCc1cc(-c2ccc(C3CCN(C(C)C)CC3)cc2)sc1C(=O)N1CC[C@H](N)C1]

### Step 1 Synthesis of methyl 5-bromo-3-methylthiophene-2-carboxylate

To a solution of 5-bromo-3-methylthiophene-2-carboxylic acid (2.2 g, 10 mmol) in MeOH (25 mL) was slowly added 5 mL con. H₂SO₄, then the mixture was stirred at 65° C. for 16 h. After the reaction was completed, H₂O (25 mL) was added and the mixture was extracted with EtOAc (25 mL*3) for 3 times. The combined organic phase was washed with brine, dried over Na₂SO₄, filtered and concentrated under reduced pressure to give the residue, which was purified by column chromatography (petroleum ether:ethyl acetate=98:2) to afford methyl 5-bromo-3-methylthiophene-2-carboxylate (2.12 g, 89% yield) as a white solid. ¹H NMR (400 MHz, CDCl3) δ 6.89 (s, 1H), 3.84 (s, 3H), 2.50 (s, 3H).

### Step 2 Synthesis of methyl 5-bromo-3-(bromomethyl)thiophene-2-carboxylate

To a solution of methyl 5-bromo-3-methylthiophene-2-carboxylate (1.2 g, 5.1 mmol) and NBS (1 g, 5.6 mmol) in CCl₄ (15 mL) was added BPO (0.12 g, 0.5 mmol), then the mixture was stirred at 90° C. for 5 h. After the reaction was completed, the solvent was removed under reduced pressure to give the residue, which was purified by column chromatography to afford methyl 5-bromo-3-(bromomethyl)thiophene-2-carboxylate (1.2 g, 74.51% yield) as a yellowish solid. ¹H NMR (400 MHz, CDCl3) δ 7.16 (s, 1H), 4.83 (s, 2H), 3.88 (s, 3H).

### Step 3 Synthesis of methyl 5-bromo-3-(methoxymethyl)thiophene-2-carboxylate

To a solution of methyl 5-bromo-3-(bromomethyl)thiophene-2-carboxylate (1.2 g, 3.8 mmol) in MeOH (20 mL) was added NaOMe (0.41 g, 7.6 mmol), then the mixture was stirred at 25° C. for 4 h. After the reaction was completed, the solvent was removed under reduced pressure to give the residue, which was purified by column chromatography to afford methyl 5-bromo-3-(methoxymethyl)thiophene-2-carboxylate (0.81 g, 78.95% yield) as a white solid. ¹H NMR (400 MHz, CDCl3) δ 7.22 (s, 1H), 4.77 (s, 2H), 3.85 (s, 3H), 3.44 (s, 3H).

### Step 4 Synthesis of 5-bromo-3-(methoxymethyl)thiophene-2-carboxylic acid

To a solution of methyl 5-bromo-3-(methoxymethyl)thiophene-2-carboxylate (810 mg, 3.06 mmol) in THF/MeOH/H₂O (v:v:v=2:2:1, 12.5 mL) was added LiOH (263.4 mg, 11.00 mmol), then the mixture was stirred at 25° C. for 2 hours. After the reaction was completed, the mixture was acidified with 1N HCl to pH=2 and extracted with EtOAc for 3 times. The combined organic phase was washed with brine, dried over Na₂SO₄ filtered, and concentrated under reduced pressure to afford 5-bromo-3-(methoxymethyl)thiophene-2-carboxylic acid (770 mg, 98.36%, yield) as an off-white solid. ¹H NMR (400 MHz, CDCl3) δ 7.23 (s, 1H), 4.77 (s, 2H), 3.47 (s, 3H).

### Step 5 Synthesis of tert-butyl (S)-(1-(5-bromo-3-(methoxymethyl)thiophene-2-carbonyl)pyrrolidin-3-yl)carbamate

To a solution of 5-bromo-3-(methoxymethyl)thiophene-2-carboxylic acid (400 mg, 1.593 mmol), tert-butyl (S)-pyrrolidin-3-ylcarbamate (296.7 mg, 1.593 mmol) and DIEA (1.03 g, 7.965 mmol) in 5 mL DMF was added T3P (1.52 g, 4.779 mmol), then the mixture was stirred at 25° C. for 16 hours. After the reaction was completed, the mixture was diluted with H₂O and extracted with EtOAc for 3 times. The combined organic phase was washed with brine, dried over Na₂SO₄, filtered and concentrated under reduced pressure to give the residue, which was purified by column chromatography using petroleum ether/ethyl acetate=3:2 as eluent to afford tert-butyl (S)-(1-(5-bromo-3-(methoxymethyl)thiophene-2-carbonyl)pyrrolidin-3-yl)carbamate (410 mg, 60.15% yield) as yellowish oil. LCMS (ESI) calc'd for C16H24BrN2O4S+ [M+H]+ m/z 419.06, 421.06, found 419.0, 421.0.

### Step 6 Synthesis of tert-butyl (S)-(1-(5-(4-(1-isopropylpiperidin-4-yl)phenyl)-3-(methoxymethyl)thiophene-2-carbonyl)pyrrolidin-3-yl)carbamate

To a solution of tert-butyl (S)-(1-(5-bromo-3-(methoxymethyl)thiophene-2-carbonyl)pyrrolidin-3-yl)carbamate (200 mg, 0.4769 mmol), 1-isopropyl-4-(4-(4,4,5,5-tetramethyl-1,3,2-dioxaborolan-2-yl)phenyl)piperidine (157.0 mg, 0.4769 mmol) and K₃PO₄ (303.7 mg, 1.4307 mmol) in 9 mL dioxane/H₂O (v:v=8:1) was added Pd(dppf)Cl₂·DCM (77.9 mg, 0.0953 mmol), then the mixture was stirred at 95° C. under N₂ for 16 h. After the reaction was completed, the solvent was removed under reduced pressure to give the residue, which was purified by column chromatography using petroleum ether/ethyl acetate=1:1 as eluent to afford tert-butyl (S)-(1-(5-(4-(1-isopropylpiperidin-4-yl)phenyl)-3-(methoxymethyl)thiophene-2-carbonyl)pyrrolidin-3-yl)carbamate (174 mg, 60.62% yield) as a yellowish solid. LCMS (ESI) calc'd for C30H44N3O4S+ [M+H]+ m/z 542.31, found 542.4.

### Step 7 Synthesis of (S)-(3-aminopyrrolidin-1-yl)(5-(4-(1-isopropylpiperidin-4-yl)phenyl)-3-(methoxymethyl)thiophen-2-yl)methanone

To a solution of tert-butyl (S)-(1-(5-(4-(1-isopropylpiperidin-4-yl)phenyl)-3-(methoxymethyl)thiophene-2-carbonyl)pyrrolidin-3-yl)carbamate (170 mg, 0.3138 mmol) in 4 ml EtOAc was added HCl (2M in EtOAc, 4 mL), then the mixture was stirred at 25° C. for 16 hours. After the completion, the solvent was removed under reduce pressure to give the residue, which was purified by prep-HPLC to afford (S)-(3-aminopyrrolidin-1-yl)(5-(4-(1-isopropylpiperidin-4-yl)phenyl)-3-(methoxymethyl)thiophen-2-yl)methanone (Compound 102, 10.6 mg, 7.58% yield) as a yellowish solid. LCMS (ESI) calc'd for C25H36N3O2S+ [M+H]+ m/z 442.25, found 442.3. ¹H NMR (400 MHz, MeOD) δ 7.63 (d, J=8.2 Hz, 2H), 7.41 (s, 1H), 7.34 (d, J=8.2 Hz, 2H), 4.56 (s, 2H), 3.97-3.93 (m, 2H), 3.87-3.71 (m, 2H), 3.65-3.63 (m, 1H), 3.58-3.53 (m, 3H), 3.41 (s, 3H), 3.21-3.14 (m, 2H), 2.96-2.90 (m, 1H), 2.43-2.35 (m, 1H), 2.16-2.00 (m, 5H), 1.40 (d, J=6.7 Hz, 6H).

### Example 78: Synthesis of (S)-(3-aminopyrrolidin-1-yl)(5-(2-ethyl-4-(piperidin-4-yl)phenyl)-3-methylthiophen-2-yl)methanone (Compound 101)

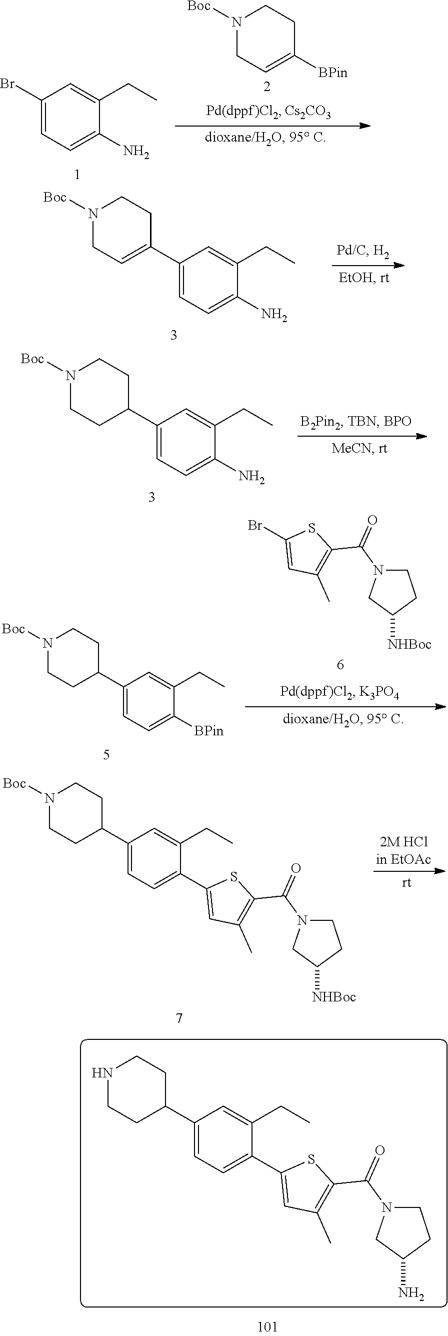

### Step 1 Synthesis of tert-butyl 4-(4-amino-3-ethylphenyl)-3,6-dihydropyridine-1(2H)-carboxylate

To a solution of 4-bromo-2-ethylaniline (3 g, 15 mmol), tert-butyl 4-(4,4,5,5-tetramethyl-1,3,2-dioxaborolan-2-yl)-3,6-dihydropyridine-1(2H)-carboxylate (5.58 g, 18 mmol) and Cs₂CO₃ (14.56 g, 45 mmol) in 100 mL dioxane/H₂O (v:v=9:1) was added Pd(dppf)Cl₂·DCM (0.61 g, 0.75 mmol), then the mixture was stirred at 95° C. under N₂ for 18 h. After the completion, the solvent was removed under reduced pressure to give the residue, which was purified by column chromatography using petroleum ether/ethyl acetate=8:1 as eluent to afford tert-butyl 4-(4-amino-3-ethylphenyl)-3,6-dihydropyridine-1(2H)-carboxylate (3.6 g, 77.33% yield) as yellow oil. LCMS (ESI) calc'd for C18H27N2O2+ [M+H]+ m/z 303.21, found 303.2.

### Step 2 Synthesis of tert-butyl 4-(4-amino-3-ethylphenyl)piperidine-1-carboxylate

To a solution of tert-butyl 4-(4-amino-3-ethylphenyl)-3,6-dihydropyridine-1(2H)-carboxylate (3.6 g, 11.6 mmol) in 25 mL EtOH was added Pd/C (0.36 g, 0.34 mmol), then the mixture was stirred at 25° C. for 16 h. After the completion, the mixture was filtered and the solvent was removed under reduced pressure to afford tert-butyl 4-(4-amino-3-ethylphenyl)piperidine-1-carboxylate (3.5 g, 99% yield) as a yellow solid. LCMS (ESI) calc'd for C18H28N2O2+ [M+H]+ m/z 305.22, found 305.2.

### Step 3 Synthesis of tert-butyl 4-(3-ethyl-4-(4,4,5,5-tetramethyl-1,3,2-dioxaborolan-2-yl)phenyl)piperidine-1-carboxylate

To a solution of tert-butyl 4-(4-amino-3-ethylphenyl)piperidine-1-carboxylate (2 g, 6.5 mmol), B₂Pin₂ (1.98 g, 7.8 mmol) and BPO (0.03 g, 0.1 mmol) in 25 mL MeCN was added TBN (1.01 g, 9.7 mmol), then the mixture was stirred at 25° C. for 2 h. After the completion, the mixture was quenched with saturated aq. Na₂SO₃ solution and extracted with EtOAc for 3 times. The combined organic phase was washed with brine, dried over Na₂SO₄, filtered and concentrated under reduced pressure to give the residue, which was purified by column chromatography using petroleum ether/ethyl acetate=10:1 as eluent to afford tert-butyl 4-(3-ethyl-4-(4,4,5,5-tetramethyl-1,3,2-dioxaborolan-2-yl)phenyl)piperidine-1-carboxylate (2 g, 52.31% yield) as a yellow solid. LCMS (ESI) calc'd for C20H31BNO4+ [M-Bu+H]+ m/z 360.23, found 360.2.

### Step 4 Synthesis of tert-butyl (S)-4-(4-(5-(3-((tert-butoxycarbonyl)amino)pyrrolidine-1-carbonyl)-4-methylthiophen-2-yl)-3-ethylphenyl)piperidine-1-carboxylate

To a solution of tert-butyl (S)-(1-(5-bromo-3-methylthiophene-2-carbonyl)pyrrolidin-3-yl)carbamate (200 mg, 0.5137 mmol), tert-butyl 4-(3-ethyl-4-(4,4,5,5-tetramethyl-1,3,2-dioxaborolan-2-yl)phenyl)piperidine-1-carboxylate (320.8 mg, 0.7705 mmol) and K₃PO₄ (327.1 mg, 1.5411 mmol) in 9 mL dioxane/H₂O (v:v=8:1) was added Pd(dppf)Cl₂·DCM (84 mg, 0.1027 mmol), then the mixture was stirred at 95° C. under N₂ for 16 h. After the completion, the solvent was removed under reduced pressure to give the residue, which was purified by column chromatography using petroleum ether/ethyl acetate=1:1 as eluent to afford tert-butyl (S)-4-(4-(5-(3-((tert-butoxycarbonyl)amino)pyrrolidine-1-carbonyl)-4-methylthiophen-2-yl)-3-ethylphenyl)piperidine-1-carboxylate (200 mg, 58.52% yield) as a yellowish solid.

LCMS (ESI) calc'd for C33H48N3O5S+ [M+H]+ m/z 598.33, found 598.3.

### Step 5 Synthesis of (S)-(3-aminopyrrolidin-1-yl)(5-(2-ethyl-4-(piperidin-4-yl)phenyl)-3-methylthiophen-2-yl)methanone

To a solution of tert-butyl (S)-4-(4-(5-(3-((tert-butoxycarbonyl)amino)pyrrolidine-1-carbonyl)-4-methylthiophen-2-yl)-3-ethylphenyl)piperidine-1-carboxylate (200 mg, 0.334 mmol) in 5 mL EtOAc was added HCl (2M in EtOAc, 4 mL), then the mixture was stirred at 25° C. for 2 h. After the completion, the solvent was removed under reduced pressure to give the residue, which was purified by prep-HPLC to afford (S)-(3-aminopyrrolidin-1-yl)(5-(2-ethyl-4-(piperidin-4-yl)phenyl)-3-methylthiophen-2-yl)methanone (Compound 101, 9.7 mg, 7.13% yield) as a white solid. LCMS (ESI) calc'd for C23H32N3OS+ [M+H]+ m/z 398.23, found 398.2.

¹H NMR (400 MHz, MeOD) δ 7.30 (d, J=7.9 Hz, 1H), 7.24 (d, J=1.6 Hz, 1H), 7.14 (dd, J=8.0, 1.8 Hz, 1H), 6.89 (s, 1H), 4.00-3.97 (m, 2H), 3.83-3.67 (m, 3H), 3.51 (d, J=12.6 Hz, 2H), 3.19-3.11 (m, 2H), 2.97-2.89 (m, 1H), 2.76 (q, J=7.5 Hz, 2H), 2.49-2.40 (tm, 1H), 2.36 (s, 3H), 2.17-2.07 (m, 3H), 1.99-1.88 (m, 2H), 1.16 (t, J=7.5 Hz, 3H).

### Example 79: Synthesis of (S)-(3-aminopyrrolidin-1-yl)(5-(3-fluoro-4-(1-isopropylpiperidin-4-yl)phenyl)-3-methylthiophen-2-yl)methanone (Compound 282)

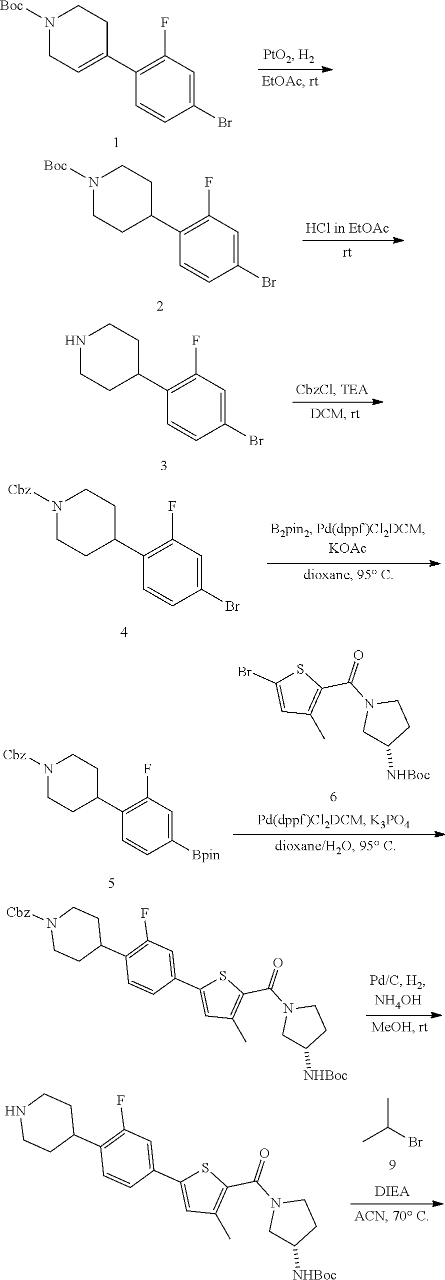

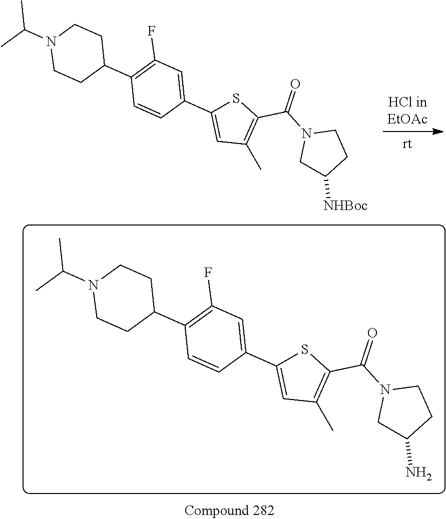 [SMILES: Cc1cc(-c2ccc(C3CCN(C(C)C)CC3)c(F)c2)sc1C(=O)N1CC[C@H](NC(=O)OC(C)(C)C)C1 ; Cc1cc(-c2ccc(C3CCN(C(C)C)CC3)c(F)c2)sc1C(=O)N1CC[C@H](N)C1]

### Step 1 Synthesis of tert-butyl 4-(4-bromo-2-fluorophenyl)piperidine-1-carboxylate

To a solution of tert-butyl 4-(4-bromo-2-fluorophenyl)-3,6-dihydropyridine-1(2H)-carboxylate (17 g, 47.6 mmol) in EtOAc (300 mL) was added PtO₂ (1.1 g, 4.7 mmol). The mixture was stirred at 25° C. under H₂ for 16 h. After completion, the mixture was filtered and concentrated.

The residue was purified by flash column chromatography using PE/EA=10/1 as eluent to afford tert-butyl 4-(4-bromo-2-fluorophenyl)piperidine-1-carboxylate (12 g, 70.17% yield) as a yellow oil.

LCMS (ESI) calculated for C₁₆H₂₁BrFNO₂⁺ [M-^{t}Bu+2H]⁺ m/z 302.01, found 302.00.

### Step 2 Synthesis of 4-(4-bromo-2-fluorophenyl)piperidine

To a solution of tert-butyl 4-(4-bromo-2-fluorophenyl)piperidine-1-carboxylate (12 g, 33.4 mmol) in EtOAc (100 mL) was added HCl (100 mL, 2N in EtOAc), the mixture was stirred at 25° C. for 16 hours. After completion, the solvent was removed under reduced pressure to afford 4-(4-bromo-2-fluorophenyl)piperidine (10 g, 92.81% yield) as a white solid.

LCMS (ESI) calculated for C₁₁H₁₃BrFN⁺ [M+H]⁺ m/z 258.02, found 258.00.

### Step 3 Synthesis of benzyl 4-(4-bromo-2-fluorophenyl)piperidine-1-carboxylate

To a solution of 4-(4-bromo-2-fluorophenyl)piperidine (12 g, 46.5 mmol) and TEA (14.1 g, 0.1395 mol) in DCM (150 mL) was added benzyl chloroformate (9.5 g, 55.8 mmol in DCM) dropwise at 0° C. Then, the reaction mixture was slowly warmed to room temperature and stirred for 16 h. After completion, the mixture was concentrated and diluted with water, the resulting mixture was extracted with EtOAc (250 mL*3). The combined organic phases were washed with brine, dried over Na₂SO₄ and filtered, the solvent was removed under reduced pressure and the residue was purified by flash column chromatography using petroleum ether/EtOAc=10/1 as eluent to afford benzyl 4-(4-bromo-2-fluorophenyl)piperidine-1-carboxylate (11 g, 60.22% yield) as a yellow oil.

LCMS (ESI) calculated for C₁₉H₁₉BrFNO₂⁺ [M+H]⁺ m/z 392.06, found 391.90.

### Step 4 Synthesis of benzyl 4-(2-fluoro-4-(4,4,5,5-tetramethyl-1,3,2-dioxaborolan-2-yl)phenyl)piperidine-1-carboxylate

To a solution of benzyl 4-(4-bromo-2-fluorophenyl)piperidine-1-carboxylate (10 g, 25.5 mmol), B₂Pin₂ (7.8 g, 30.6 mmol) and KOAc (7.5 g, 76.5 mmol) in 1,4-dioxane (150 mL) was added Pd(dppf)Cl₂DCM (2.1 g, 2.6 mmol), the mixture was stirred at 95° C. under N₂ for 16 h. After completion, the mixture was concentrated and the residue was purified by flash column chromatography (PE/EA=10/1) to afford benzyl 4-(2-fluoro-4-(4,4,5,5-tetramethyl-1,3,2-dioxaborolan-2-yl)phenyl)piperidine-1-carboxylate (10.8 g, 86.67% yield) as a yellow oil.

LCMS (ESI) calculated for C₂₅H₃₁BFNO₄⁺ [M+H]⁺ m/z 440.23, found 439.85.

### Step 5 Synthesis of benzyl (S)-4-(4-(5-(3-((tert-butoxycarbonyl)amino)pyrrolidine-1-carbonyl)-4-methylthiophen-2-yl)-2-fluorophenyl)piperidine-1-carboxylate

To a solution of benzyl 4-(2-fluoro-4-(4,4,5,5-tetramethyl-1,3,2-dioxaborolan-2-yl)phenyl)piperidine-1-carboxylate (4 g, 9.1 mol), tert-butyl (S)-(1-(5-bromo-3-methylthiophene-2-carbonyl)pyrrolidin-3-yl)carbamate (4.3 g, 10.9 mmol) and K₃PO₄ (5.8 g, 27.3 mmol) in 1,4-dioxane/H₂O (220 mL, v/v=8/1) was added Pd(dppf)Cl₂DCM (0.7 g, 0.91 mmol), the mixture was stirred at 95° C. under N₂ for 16 h. After completion, the mixture was concentrated and the residue was purified by flash column chromatography (PE/EA=1/1) to afford benzyl (S)-4-(4-(5-(3-((tert-butoxycarbonyl)amino)pyrrolidine-1-carbonyl)-4-methylthiophen-2-yl)-2-fluorophenyl)piperidine-1-carboxylate (3.3 g, 58.24% yield) as a yellow oil. LCMS (ESI) calculated for C₃₄H₄₀FN₃O₅S [M+H]⁺ m/z 622.17, found 622.05.

### Step 6 Synthesis of tert-butyl (S)-(1-(5-(3-fluoro-4-(piperidin-4-yl)phenyl)-3-methylthiophene-2-carbonyl)pyrrolidin-3-yl)carbamate

To a solution of benzyl (S)-4-(4-(5-(3-((tert-butoxycarbonyl)amino)pyrrolidine-1-carbonyl)-4-methylthiophen-2-yl)-2-fluorophenyl)piperidine-1-carboxylate (6 g, 9.6 mmol) and NH₄OH aq. (0.2 mL) in MeOH (300 mL) was added Pd/C (0.6 g), the mixture was stirred at 25° C. under H₂ for 16 h. After completion, the mixture was filtered and concentrated. The residue was purified by flash column chromatography to afford tert-butyl (S)-(1-(5-(3-fluoro-4-(piperidin-4-yl)phenyl)-3-methylthiophene-2-carbonyl)pyrrolidin-3-yl)carbamate (4 g, 85.42% yield) as a yellow oil.

LCMS (ESI) calculated for C₂₆H₃₄FN₃O₃S+ [M+H]⁺ m/z 488.23, found 488.40.

### Step 7 Synthesis of tert-butyl (S)-(1-(5-(3-fluoro-4-(1-isopropylpiperidin-4-yl)phenyl)-3-methylthiophene-2-carbonyl)pyrrolidin-3-yl)carbamate

To a solution of tert-butyl (S)-(1-(5-(3-fluoro-4-(piperidin-4-yl)phenyl)-3-methylthiophene-2-carbonyl)pyrrolidin-3-yl)carbamate (200 mg, 0.4101 mmol) and DIEA (159 mg, 1.2303 mmol) in MeCN (5 mL) was added 2-bromopropane (101 mg, 0.8202 mmol), the mixture was stirred at 70° C. for 16 h. After completion, the mixture was concentrated and the residue was purified by flash column chromatography using DCM/MeOH=10/1 as eluent to afford tert-butyl (S)-(1-(5-(3-fluoro-4-(1-isopropylpiperidin-4-yl)phenyl)-3-methylthiophene-2-carbonyl)pyrrolidin-3-yl)carbamate (160 mg, 73.66% yield) as a white solid.

LCMS (ESI) calculated for C₂₉H₄₀FN₃O₃S⁺ [M+H]⁺ m/z 530.28, found 530.45.

### Step 8 Synthesis of (S)-(3-aminopyrrolidin-1-yl)(5-(3-fluoro-4-(1-isopropylpiperidin-4-yl)phenyl)-3-methylthiophen-2-yl)methanone (Compound 282)

To a solution of tert-butyl (S)-(1-(5-(3-fluoro-4-(1-isopropylpiperidin-4-yl)phenyl)-3-methylthiophene-2-carbonyl)pyrrolidin-3-yl)carbamate (160 mg, 0.3021 mmol) in EtOAc (10 mL) was added HCl (5 mL, 2N in EtOAc), the mixture was stirred at 25° C. for 16 hours. After completion, the solvent was removed under reduced pressure to afford (S)-(3-aminopyrrolidin-1-yl)(5-(3-fluoro-4-(1-isopropylpiperidin-4-yl)phenyl)-3-methylthiophen-2-yl)methanone (98 mg, 75.5% yield) as a white solid.

LCMS (ESI) calculated for C₂₄H₃₂FN₃OS⁺ [M+H]⁺ m/z 430.23, found 430.20.

¹H NMR (400 MHz, MeOD) δ 7.46 (d, J=7.9 Hz, 1H), 7.42-7.37 (m, 2H), 7.30 (s, 1H), 4.04-3.97 (m, 2H), 3.88-3.69 (m, 4H), 3.59 (d, J=10.9 Hz, 3H), 3.25 (t, J=10.1 Hz, 3H), 2.53-2.41 (m, 1H), 2.35 (s, 3H), 2.17 (dd, J=26.9, 12.6 Hz, 5H), 1.43 (s, 3H), 1.38 (t, J=6.4 Hz, 2H).

### Example 80: Synthesis of (S)-(3-aminopyrrolidin-1-yl)(3-chloro-5-(3-fluoro-4-(1-isopropylpiperidin-4-yl)phenyl)thiophen-2-yl)methanone (Compound 284)

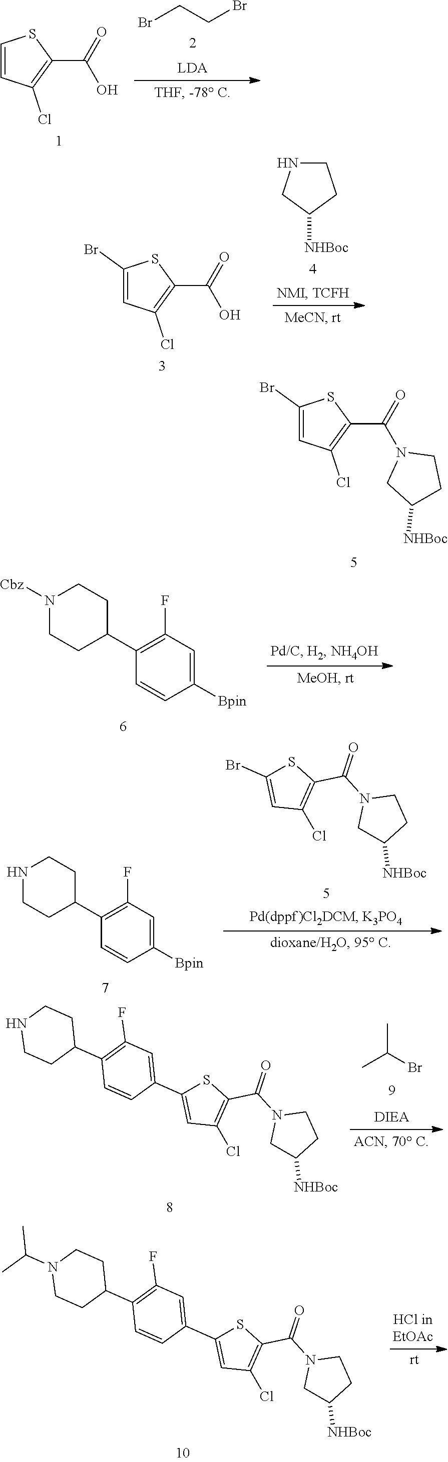

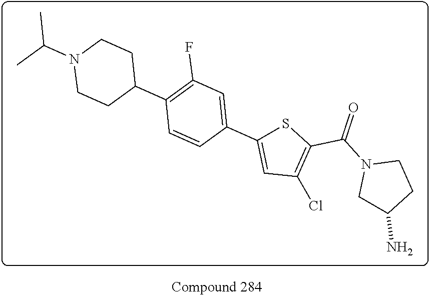 [SMILES: CC(C)N1CCC(c2ccc(-c3cc(Cl)c(C(=O)N4CC[C@H](N)C4)s3)cc2F)CC1]

### Step 1 Synthesis of 5-bromo-3-chlorothiophene-2-carboxylic acid

To a solution of 3-chlorothiophene-2-carboxylic acid (5 g, 30.8 mmol) in THF (50 mL) was added LDA (31 mL, 2N in THF) at −78° C. under N₂ atmosphere for 1 hour. Then 1,2-Dibromoethane (5.3 mL) was added, and the mixture was stirred at −78° C. for 1 hours and at room temperature for 16 h. After completion, the mixture was poured into 2M HCl aq. solution and extracted with EtOAc (20 mL*3). The organic layers were washed with brine, dried over Na₂SO₄, filtered, and concentrated. The residue was purified by column chromatography using petroleum ether/ethyl acetate=2/1 as eluent to afford 5-bromo-3-chlorothiophene-2-carboxylic acid (5 g, 34% yield) as a yellow solid.

LCMS (ESI) calcd for C₅H₃BrClO₂S⁺ [M−H]⁺ m/z 238.86, found 238.8.

### Step 2 Synthesis of tert-butyl (S)-(1-(5-bromo-3-chlorothiophene-2-carbonyl)pyrrolidin-3-yl)carbamate

To a solution of 5-bromo-3-chlorothiophene-2-carboxylic acid (5 g, 20.7 mmol) and tert-butyl (S)-pyrrolidin-3-ylcarbamate (4.24 g, 22.8 mmol) and NMI (5.10 g, 62.1 mmol) in MeCN (80 mL) was added TCFH (8.71 g, 31.1 mmol), the mixture was stirred at 25° C. for 16 h. After completion, the solvent was removed under reduced pressure and the residue was purified by column chromatography using petroleum ether/ethyl acetate=1/1 as eluent to afford tert-butyl (S)-(1-(5-bromo-3-chlorothiophene-2-carbonyl)pyrrolidin-3-yl)carbamate (3 g, 32% yield) as a yellow oil.

LCMS (ESI) calcd for C₁₄H₁₈BrClN₂O₃S⁺ [M+H]⁺ m/z 408.99, found 408.9.

### Step 3 Synthesis of 4-(2-fluoro-4-(4,4,5,5-tetramethyl-1,3,2-dioxaborolan-2-yl)phenyl)piperidine

To a solution of benzyl 4-(2-fluoro-4-(4,4,5,5-tetramethyl-1,3,2-dioxaborolan-2-yl)phenyl)piperidine-1-carboxylate (6 g, 13.7 mmol) in MeOH (150 mL) was added Pd/C (0.29 g), the mixture was stirred at 25° C. under H₂ for 16 hours. After completion, the mixture was filtered and the filtrate was concentrated under reduced pressure to afford 4-(2-fluoro-4-(4,4,5,5-tetramethyl-1,3,2-dioxaborolan-2-yl)phenyl)piperidine (4 g, 91% yield) as a yellow oil.

LCMS (ESI) calcd for C₁₇H₂₅BFNO₂⁺ [M+H]⁺ m/z 306.20 found 306.1

### Step 4 Synthesis of tert-butyl (S)-(1-(3-chloro-5-(3-fluoro-4-(piperidin-4-yl)phenyl)thiophene-2-carbonyl)pyrrolidin-3-yl)carbamate

To a solution of tert-butyl (S)-(1-(5-bromo-3-chlorothiophene-2-carbonyl)pyrrolidin-3-yl)carbamate (2 g, 4.9 mmol), 4-(2-fluoro-4-(4,4,5,5-tetramethyl-1,3,2-dioxaborolan-2-yl)phenyl)piperidine (2.54 g, 8.3 mmol) and K₃PO₄ (3.12 g, 14.7 mmol) in 1,4-dioxane/H₂O (44 mL, v/v=10/1) was added Pd(dppf)Cl₂DCM (0.4 g, 0.49 mmol), the mixture was stirred at 95° C. under N₂ for 16 hours. After completion, the solvent was removed under reduced pressure and the residue was purified by column chromatography using dichloromethane/methanol=9/1 as eluent to afford tert-butyl (S)-(1-(3-chloro-5-(3-fluoro-4-(piperidin-4-yl)phenyl)thiophene-2-carbonyl)pyrrolidin-3-yl)carbamate (1.8 g, 61% yield) as a brown solid.

LCMS (ESI) calcd for C₂₅H₃₁ClFN₃O₃S⁺ [M+H]⁺ m/z 508.18, found 508.2.

### Step 5 Synthesis of tert-butyl (S)-(1-(3-chloro-5-(3-fluoro-4-(1-isopropylpiperidin-4-yl)phenyl)thiophene-2-carbonyl)pyrrolidin-3-yl)carbamate

To a solution of tert-butyl (S)-(1-(3-chloro-5-(3-fluoro-4-(piperidin-4-yl)phenyl)thiophene-2-carbonyl)pyrrolidin-3-yl)carbamate (180 mg, 0.35 mmol) and 2-bromopropane (131 mg, 1.1 mmol) in MeCN (5 mL) was added K₂CO₃ (147 mg, 1.1 mmol), the mixture was stirred at 70° C. for 16 hours. After completion, the mixture was concentrated and the residue was purified by Prep-HPLC (0.1% TFA) to afford tert-butyl (S)-(1-(3-chloro-5-(3-fluoro-4-(1-isopropylpiperidin-4-yl)phenyl)thiophene-2-carbonyl)pyrrolidin-3-yl)carbamate (120 mg, 60% yield) as a yellow solid.

LCMS (ESI) calcd for C₂₈H₃₇ClFN₃O₃S⁺ [M+H]⁺ m/z 550.22, found 550.2.

### Step 6 Synthesis of (S)-(3-aminopyrrolidin-1-yl)(3-chloro-5-(3-fluoro-4-(1-isopropylpiperidin-4-yl)phenyl)thiophen-2-yl)methanone (Compound 284)

To a solution of tert-butyl (S)-(1-(3-chloro-5-(3-fluoro-4-(1-isopropylpiperidin-4-yl)phenyl)thiophene-2-carbonyl)pyrrolidin-3-yl)carbamate (120 mg, 0.22 mmol) in EtOAc (5 mL) was added HCl (5 mL, 2N in EtOAc), the mixture was stirred at 25° C. for 16 h. After completion, the solvent was removed under reduced pressure afford (S)-(3-aminopyrrolidin-1-yl)(3-chloro-5-(3-fluoro-4-(1-isopropylpiperidin-4-yl)phenyl)thiophen-2-yl)methanone (50 mg, 50% yield) as a yellow solid.

LCMS (ESI) calcd for C₂₃H₂₉ClFN₃OS⁺ [M+H]⁺ m/z 450.17, found 450.1.

¹H NMR (400 MHz, MeOD) δ 7.51-7.40 (m, 4H), 4.10-3.97 (m, 2H), 3.88-3.70 (m, 3H), 3.59 (d, J=8.4 Hz, 3H), 3.30-3.18 (m, 3H), 2.47 (s, 1H), 2.26-2.10 (m, 5H), 1.43 (d, J=6.0 Hz, 6H).

| Compound | Name | ¹H NMR | MS |
|---|---|---|---|
| 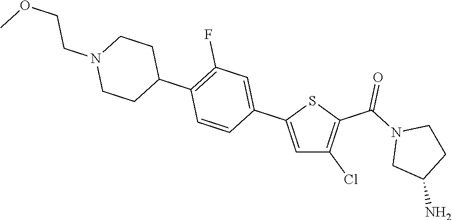 [SMILES: COCCN1CCC(c2ccc(-c3cc(Cl)c(C(=O)N4CC[C@H](N)C4)s3)cc2F)CC1] | (S)-(3-aminopyrrolidin-1- yl)(3-chloro-5-(3-fluoro- 4-(1-(2-methoxyethyl) piperidin-4-yl)phenyl) thiophen-2-yl)methanone | ¹H NMR (400 MHz, MeOD) δ 7.54-7.38 (m, 4H), 4.02 (d, J = 8.5 Hz, 2H), 3.87-3.68 (m, 7H), 3.44 (d, J = 4.3 Hz, 3H), 3.39 (s, 2H), 3.24 (d, J = 9.8 Hz, 3H), 2.47 (s, 1H), 2.13 (s, 5H). | 468.2 [M + H]⁺ |
| Compound 283 | | | |
| 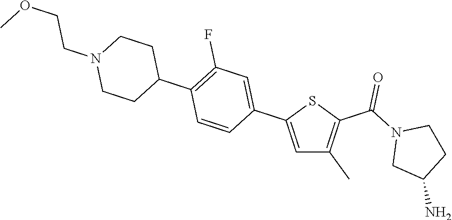 [SMILES: COCCN1CCC(c2ccc(-c3cc(C)c(C(=O)N4CC[C@H](N)C4)s3)cc2F)CC1] | (S)-(3-aminopyrrolidin-1- yl)(5-(3-fluoro-4-(1-(2- methoxyethyl)piperidin- 4-yl)phenyl)-3-methyl- thiophen-2-yl)methanone | ¹H NMR (400 MHz, MeOD) δ 7.47-7.44 (m, 1H), 7.42- 7.36 (m, 2H), 7.30 (s, 1H), 4.04-3.97 (m, 2H), 3.86 (dd, J = 12.4, 6.7 Hz, 1H), 3.76 (dt, J = 19.5, 6.3 Hz, 6H), 3.43 (s, 3H), 3.41-3.38 (m, 2H), 3.28-3.19 (m, 3H), 2.46 (dd, J = 13.4, 6.3 Hz, 1H), 2.35 (s, 3H), 2.13 (m, 5H). | 446.15 [M + H]⁺ |
| Compound 276 | | | |
| 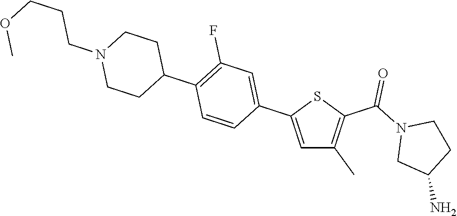 [SMILES: COCCCN1CCC(c2ccc(-c3cc(C)c(C(=O)N4CC[C@H](N)C4)s3)cc2F)CC1] | (S)-(3-aminopyrrolidin-1- yl)(5-(3-fluoro-4-(1-(3- methoxypropyl)piperidin- 4-yl)phenyl)-3-methyl- thiophen-2-yl) methanone | ¹H NMR (400 MHz, MeOD) δ 7.45 (dd, J = 8.0, 1.6 Hz, 1H), 7.40 (dd, J = 11.7, 1.5 Hz, 1H), 7.35 (t, J = 7.9 Hz, 1H), 7.29 (s, 1H), 4.02-3.95 (m, 2H), 3.88-3.81 (m, 1H), 3.81- 3.65 (m, 5H), 3.52 (t, J = 5.7 Hz, 2H), 3.32-3.30 (m, 1H), 3.28-3.20 (m, 3H), 3.18-3.11 (m, 2H), 2.48-2.35 (m, 1H), 2.35 (s, 3H), 2.19-2.00 (m, 8H). | 460.15 [M + H]⁺ |
| Compound 275 | | | |
| 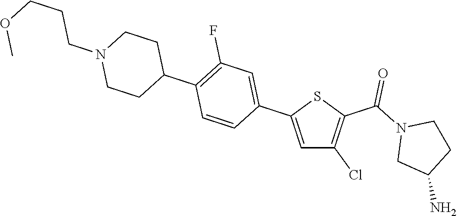 [SMILES: COCCCN1CCC(c2ccc(-c3cc(Cl)c(C(=O)N4CC[C@H](N)C4)s3)cc2F)CC1] | (S)-(3-aminopyrrolidin- 1-yl)(3-chloro-5-(3- fluoro-4-(1-(3-methoxy- propyl)piperidin-4-yl) phenyl)thiophen-2- yl)methanone | ¹H NMR (400 MHz, MeOD) δ 7.50-7.40 (m, 4H), 4.11-3.96 (m, 2H), 3.86-3.69 (m, 5H), 3.53 (t, J = 5.2 Hz, 2H), 3.36 (s, 3H), 3.30-3.23 (m, 3H), 3.18 (t, J = 11.2 Hz, 2H), 2.47 (s, 1H), 2.20-2.05 (m, 7H). | 480.2 [M + H]⁺ |
| Compound 268 | | | |
| 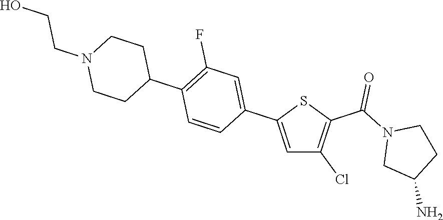 [SMILES: N[C@H]1CCN(C(=O)c2sc(-c3ccc(C4CCN(CCO)CC4)c(F)c3)cc2Cl)C1] | (S)-(3-aminopyrrolidin- 1-yl)(3-chloro-5-(3- fluoro-4-(1-(2-hydroxy- ethyl)piperidin-4-yl) phenyl)thiophen-2- yl)methanone | ¹H NMR (400 MHz, MeOD) δ 7.52-7.37 (m, 4H), 4.08-3.98 (m, 2H), 3.96-3.89 (m, 2H), 3.88-3.65 (m, 5H), 3.30-3.15 (m, 5H), 2.53-2.41 (m, 1H), 2.24-2.02 (m, 5H). | 452.1 [M + H]⁺ |
| Compound 263 | | | |
| 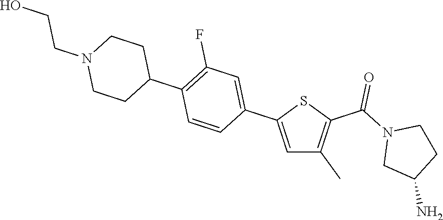 [SMILES: Cc1cc(-c2ccc(C3CCN(CCO)CC3)c(F)c2)sc1C(=O)N1CC[C@H](N)C1] | (S)-(3-aminopyrrolidin- 1-yl)(5-(3-fluoro-4-(1-(2- hydroxyethyl)piperidin- 4-yl)phenyl)-3-methyl- thiophen-2-yl) methanone | ¹H NMR (400 MHz, MeOD) δ 7.47 (d, J = 7.6 Hz, 1H), 7.43-7.35 (m, 2H), 7.30 (s, 1H), 4.05-3.96 (m, 2H), 3.96- 3.90 (m, 2H), 3.88-3.81 (m, 1H), 3.80-3.68 (m, 4H), 3.30- 3.18 (m, 5H), 2.51-2.40 (m, 1H), 2.36 (s, 3H), 2.23-2.07 (m, 5H). | 432.2 [M + H]⁺ |
| Compound 262 | | | |
| 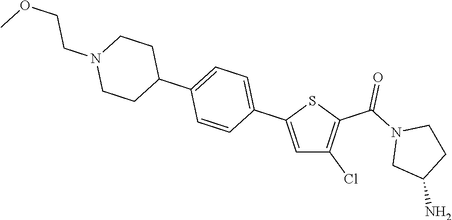 [SMILES: COCCN1CCC(c2ccc(-c3cc(Cl)c(C(=O)N4CC[C@H](N)C4)s3)cc2)CC1] | (S)-(3-aminopyrrolidin-1- yl)(3-chloro-5-(4-(1-(2- methoxyethyl)piperidin- 4-yl)phenyl)thiophen-2- yl)methanone | ¹H NMR (400 MHz, MeOD) δ 7.64 (d, J = 8.2 Hz, 2H), 7.37 (s, 1H), 7.35 (s, 2H), 4.01 (d, J = 8.6 Hz, 2H), 3.82-3.71 (m, 6H), 3.43 (s, 3H), 3.39- 3.36 (m, 2H), 3.17 (dd, J = 12.7, 10.0 Hz, 2H), 2.99-2.90 (m, 1H), 2.46 (dd, J = 13.5, 6.7 Hz, 1H), 2.20-1.97 (m, 6H). | 448.10 [M + H]⁺ |
| Compound 258 | | | |
| 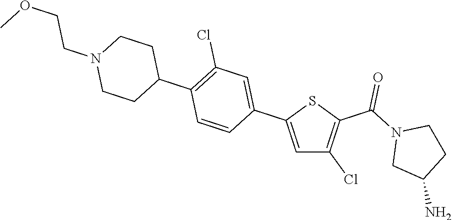 [SMILES: COCCN1CCC(c2ccc(-c3cc(Cl)c(C(=O)N4CC[C@H](N)C4)s3)cc2Cl)CC1] | (S)-(3-aminopyrrolidin-1- yl)(3-chloro-5-(3-chloro- 4-(1-(2-methoxyethyl) piperidin-4-yl)phenyl) thiophen-2-yl)methanone | ¹H NMR (400 MHz, MeOD) δ 7.75 (d, J = 2.0 Hz, 1H), 7.65-7.61 (m, 1H), 7.45-7.40 (m, 2H), 4.05-3.97 (m, 2H), 3.88-3.65 (m, 8H), 3.43 (s, 3H), 3.41-3.37 (m, 2H), 3.27- 3.19 (m, 2H), 2.52-2.40 (m, 1H), 2.15 (d, J = 13.2 Hz, 3H), 2.10-2.01 (m, 2H). | 482.2 [M + H]⁺ |
| Compound 253 | | | |
| 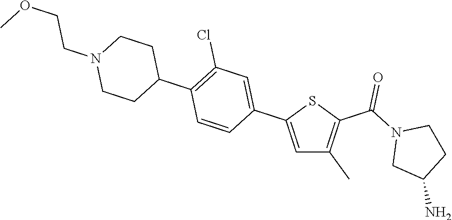 [SMILES: COCCN1CCC(c2ccc(-c3cc(C)c(C(=O)N4CC[C@H](N)C4)s3)cc2Cl)CC1] | (S)-(3-aminopyrrolidin-1- yl)(5-(3-chloro-4-(1-(2- methoxyethyl)piperidin- 4-yl)phenyl)-3-methyl- thiophen-2-yl)methanone | ¹H NMR (400 MHz, MeOD) δ 7.70 (d, J = 2.0 Hz, 1H), 7.62-7.58 (m, 1H), 7.39 (d, J = 8.0 Hz, 1H), 7.30 (s, 1H), 4.03-3.95 (m, 2H), 3.88-3.81 (m, 1H), 3.79-3.65 (m, 7H), 3.43 (s, 3H), 3.40-3.37 (m, 2H), 3.27-3.18 (m, 2H), 2.49- 2.39 (m, 1H), 2.35 (s, 3H), 2.17-2.03 (m, 5H). | 462.2 [M + H]⁺ |
| Compound 248 | | | |

### Example 81: Synthesis of (S)-(3-aminopyrrolidin-1-yl)(5-(4-(1-ethylpiperidin-4-yl)-3-fluorophenyl)-3-methylthiophen-2-yl)methanone (Compound 281)

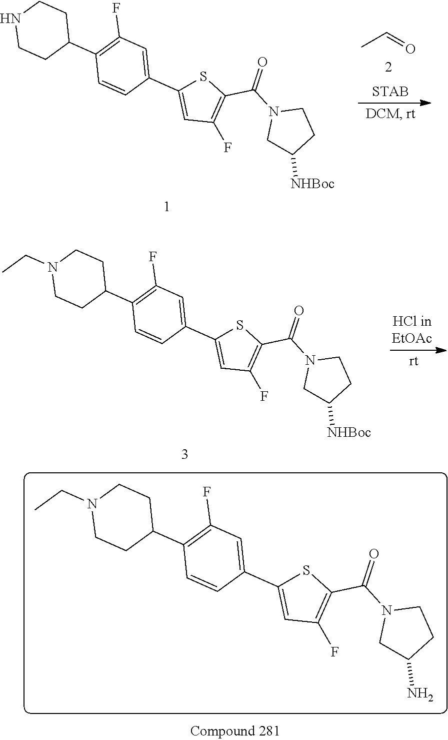

### Step 1 Synthesis of tert-butyl (S)-(1-(5-(4-(1-ethylpiperidin-4-yl)-3-fluorophenyl)-3-methylthiophene-2-carbonyl)pyrrolidin-3-yl)carbamate

To a mixture of tert-butyl (S)-(1-(5-(3-fluoro-4-(piperidin-4-yl)phenyl)-3-methylthiophene-2-carbonyl)pyrrolidin-3-yl)carbamate (200 mg, 0.4101 mmol) and acetaldehyde (54 mg, 1.2303 mmol) in MeOH (5 mL) was added NaBH₃CN (77 mg, 1.2303 mmol), the mixture was stirred at 25° C. for 16 hours. After completion, the mixture was concentrated to dryness. The crude product was purified by column chromatography to afford tert-butyl (S)-(1-(5-(4-(1-ethylpiperidin-4-yl)-3-fluorophenyl)-3-methylthiophene-2-carbonyl)pyrrolidin-3-yl)carbamate (80 mg, 37.82% yield) as a white solid.

LCMS (ESI) calculated for C₂₈H₃₈FN₃O₃S⁺ [M+H]⁺ m/z 516.26, found 516.30.

### Step 2 Synthesis of (S)-(3-aminopyrrolidin-1-yl)(5-(4-(1-ethylpiperidin-4-yl)-3-fluorophenyl)-3-methylthiophen-2-yl)methanone (Compound 281)

To a solution of tert-butyl (S)-(1-(5-(4-(1-ethylpiperidin-4-yl)-3-fluorophenyl)-3-methylthiophene-2-carbonyl)pyrrolidin-3-yl)carbamate (80 mg, 0.1551 mmol) in EtOAc (2 mL) was added HCl (5 mL, 2N in EtOAc), the mixture was stirred at 25° C. for 16 hours. After completion, the solvent was removed under reduced pressure to afford (S)-(3-aminopyrrolidin-1-yl)(5-(4-(1-ethylpiperidin-4-yl)-3-fluorophenyl)-3-methylthiophen-2-yl)methanone (58 mg, 90.01% yield) as a yellow solid.

LCMS (ESI) calculated for C₂₃H₃₀FN₃OS⁺ [M+H]⁺ m/z 416.21, found 416.15.

¹H NMR (400 MHz, MeOD) δ 7.47 (dd, J=8.0, 1.5 Hz, 1H), 7.43-7.36 (m, 2H), 7.30 (s, 1H), 4.00 (d, J=9.1 Hz, 2H), 3.84 (dd, J=7.4, 4.2 Hz, 1H), 3.81-3.75 (m, 1H), 3.70 (d, J=9.6 Hz, 3H), 3.24 (dd, J=14.6, 7.3 Hz, 3H), 3.14 (dd, J=16.3, 12.1 Hz, 2H), 2.45 (dd, J=13.7, 6.6 Hz, 1H), 2.35 (s, 3H), 2.14 (d, J=5.5 Hz, 5H), 1.40 (t, J=7.3 Hz, 3H).

| Compound | Name | ¹H NMR | MS |
|---|---|---|---|
| 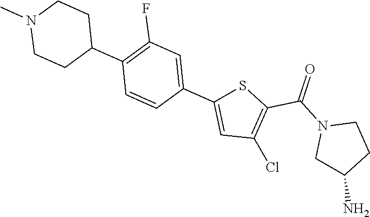 [SMILES: CN1CCC(c2ccc(-c3cc(Cl)c(C(=O)N4CC[C@H](N)C4)s3)cc2F)CC1] | (S)-(3- aminopyrrolidin- 1-yl)(3- chloro-5-(3- fluoro-4-(1- methyl- piperidin-4-yl) phenyl) thiophen-2-yl) methanone | ¹H NMR (400 MHz, MeOD) δ 7.50-7.40 (m, 4H), 4.02 (d, J = 8.8 Hz, 2H), 3.88-3.76 (m, 2H), 3.71 (d, J = 8.8 Hz, 1H), 3.64 (d, J = 11.6 Hz, 2H), 3.22 (s, 3H), 2.93 (s, 3H), 2.47 (s, 1H), 2.21-2.07 (m, 5H). | 422.2 [M + H]⁺ |
| Compound 280 | | | |
| 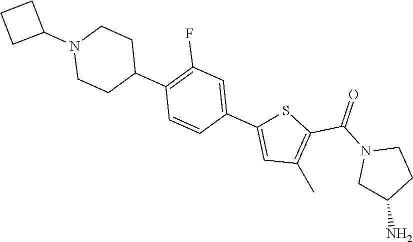 [SMILES: Cc1cc(-c2ccc(C3CCN(C4CCC4)CC3)c(F)c2)sc1C(=O)N1CC[C@H](N)C1] | (S)-(3- aminopyrrolidin- 1-yl)(5-(4-(1- cyclobutyl- piperidin- 4-yl)-3- fluorophenyl)- 3-methyl- thiophen-2-yl) methanone | ¹H NMR (400 MHz, MeOD) δ 7.45 (d, J = 7.1 Hz, 1H), 7.39 (d, J = 10.09 Hz, 2H), 7.30 (s, 1H), 4.01 (d, J = 8.6 Hz, 2H), 3.84 (s, 2H), 3.72 (d, J = 8.7 Hz, 2H), 3.59 (d, J = 9.5 Hz, 2H), 3.34-3.29 (m, 2H), 3.23 (s, 1H), 2.98 (s, 2H), 2.35 (s, 6H), 2.13 (d, J = 16.8 Hz, 6H), 1.98-1.80 (m, 3H). | 442.15 [M + H]⁺ |
| Compound 279 | | | |
| 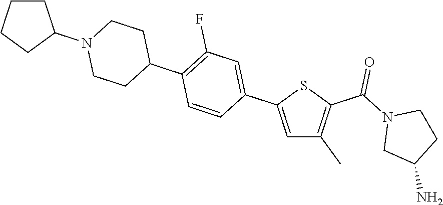 [SMILES: Cc1cc(-c2ccc(C3CCN(C4CCCC4)CC3)c(F)c2)sc1C(=O)N1CC[C@H](N)C1] | (S)-(3- aminopyrrolidin- 1-yl)(5-(4- (1-cyclo- pentylpiperidin- 4-yl)-3- fluorophenyl)- 3- methylthiophen- 2- yl)methanone | ¹H NMR (400 MHz, MeOD) δ 7.45 (d, J = 7.7 Hz, 1H), 7.39 (dd, J = 13.6, 5.9 Hz, 2H), 7.30 (s, 1H), 4.04-3.97 (m, 2H), 3.88-3.81 (m, 1H), 3.73 (t, J = 9.0 Hz, 4H), 3.57 (s, 1H), 3.20 (dd, J = 24.3, 12.5 Hz, 3H), 2.46 (d, J = 6.4 Hz, 1H), 2.35 (s, 3H), 2.15 (d, J = 23.9 Hz, 7H), 1.86 (s, 4H), 1.70 (s, 2H). | 456.2 [M + H]⁺ |
| Compound 278 | | | |
| 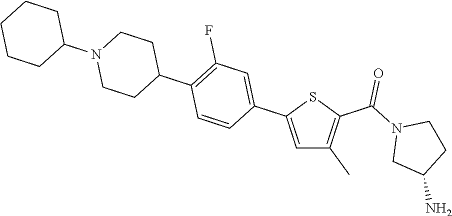 [SMILES: Cc1cc(-c2ccc(C3CCN(C4CCCCC4)CC3)c(F)c2)sc1C(=O)N1CC[C@H](N)C1] | (S)-(3- aminopyrrolidin- 1-yl)(5-(4-(1- cyclohexyl- piperidin-4-yl)- 3-fluoro- phenyl)-3- methylthiophen- 2-yl) methanone | ¹H NMR (400 MHz, MeOD) δ 7.47 (dd, J = 8.0, 1.5 Hz, 1H), 7.42-7.36 (m, 2H), 7.30 (s, 1H), 4.01-3.97 (m, 2H), 3.87- 3.81 (m, 1H), 3.80-3.75 (m, 1H), 3.73- 3.66 (m, 2H), 3.61-3.59 (m, 1H), 3.27- 3.18 (m, 3H), 2.51-2.43 (m, 1H), 2.35 (s, 3H), 2.25-2.09 (m, 7H), 1.96 (d, J = 13.0 Hz, 2H), 1.74 (d, J = 13.0 Hz, 1H), 1.56 (dd, J = 22.1, 9.9 Hz, 2H), 1.47-1.35 (m, 3H), 1.29-1.19 (m, 1H). | 470.25 [M + H]⁺ |
| Compound 277 | | | |
| 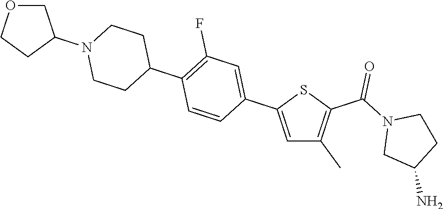 [SMILES: Cc1cc(-c2ccc(C3CCN(C4CCOC4)CC3)c(F)c2)sc1C(=O)N1CC[C@H](N)C1] | ((S)-3- aminopyrrolidin- 1-yl)(5-(3- fluoro-4-(1- (tetrahydrofuran- 3-yl)piperidin- 4-yl)phenyl)-3- methyl- thiophen-2-yl) methanone | ¹H NMR (400 MHz, MeOD) δ 7.47 (d, J = 8.0 Hz, 1H), 7.39 (dd, J = 15.3, 9.7 Hz, 2H), 7.30 (s, 1H), 4.21 (dd, J = 10.8, 3.2 Hz, 1H), 4.13-4.07 (m, 1H), 4.06-3.96 (m, 3H), 3.90 (dd, J = 10.9, 6.4 Hz, 1H), 3.85-3.75 (m, 3H), 3.70 (dd, J = 19.0, 10.4 Hz, 3H), 3.25 (d, J = 20.6 Hz, 3H), 2.48-2.39 (m, 2H), 2.35 (s, 3H), 2.32-2.25 (m, 1H), 2.20- 2.07 (m, 5H). | 458.20 [M + H]⁺ |
| Compound 274 | | | |
| 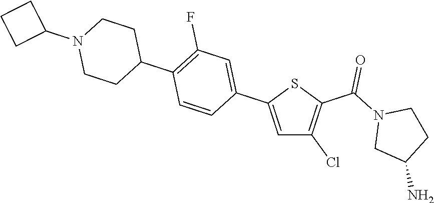 [SMILES: N[C@H]1CCN(C(=O)c2sc(-c3ccc(C4CCN(C5CCC5)CC4)c(F)c3)cc2Cl)C1] | (S)-(3-amino- pyrrolidin-1-yl) (3-chloro-5-(4- (1-cyclobutyl- piperidin-4-yl)- 3-fluorophenyl) thiophen-2- yl)methanone | ¹H NMR (400 MHz, MeOD) δ 7.51- 7.39 (m, 4H), 4.03 (d, J = 9.6 Hz, 2H), 3.88-3.68 (m, 4H), 3.60 (d, J = 11.2 Hz, 2H), 3.29-3.20 (m, 1H), 2.99 (t, J = 10.8 Hz, 2H), 2.47 (s, 1H), 2.44- 2.31 (m, 4H), 2.24-2.05 (m, 5H), 1.97- 1.82 (m, 1H). | 462.2 [M + H]⁺ |
| Compound 271 | | | |
| 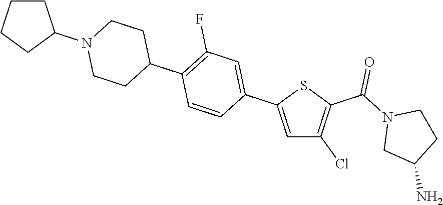 [SMILES: N[C@H]1CCN(C(=O)c2sc(-c3ccc(C4CCN(C5CCCC5)CC4)c(F)c3)cc2Cl)C1] | (S)-(3-amino- pyrrolidin-1-yl) (3-chloro-5-(4- (1-cyclopentyl- piperidin-4-yl)- 3-fluorophenyl) thiophen-2-yl) methanone | ¹H NMR (400 MHz, MeOD) δ 7.59- 7.32 (m, 4H), 4.02 (d, J = 8.2 Hz, 2H), 3.75 (dd, J = 31.1, 19.6 Hz, 5H), 3.57 (s, 1H), 3.22 (d, J = 35.1 Hz, 3H), 2.47 (s, 1H), 2.17 (d, J = 22.4 Hz, 7H), 1.94-1.66 (m, 6H). | 476.2 [M + H]⁺ |
| Compound 270 | | | |
| 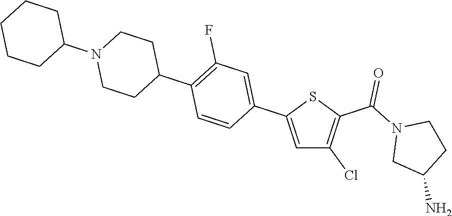 [SMILES: N[C@H]1CCN(C(=O)c2sc(-c3ccc(C4CCN(C5CCCCC5)CC4)c(F)c3)cc2Cl)C1] | (S)-(3-amino- pyrrolidin-1-yl) (3-chloro-5-(4- (1-cyclohexyl- piperidin-4-yl)- 3-fluorophenyl) thiophen-2-yl) methanone | ¹H NMR (400 MHz, MeOD) δ 7.55- 7.32 (m, 4H), 4.02 (d, J = 7.4 Hz, 2H), 3.81 (s, 2H), 3.71 (d, J = 9.3 Hz, 1H), 3.62 (s, 2H), 3.26 (s, 3H), 2.47 (s, 1H), 2.16 (s, 7H), 1.97 (d, J = 11.6 Hz, 2H), 1.74 (d, J = 12.8 Hz, 1H), 1.63-1.51 (m, 2H), 1.43 (d, J = 12.9 Hz, 2H), 1.28 (d, J = 6.9 Hz, 2H). | 490.2 [M + H]⁺ |
| Compound 269 | | | |
| 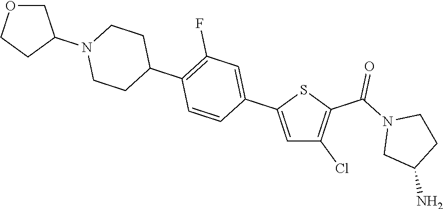 [SMILES: N[C@H]1CCN(C(=O)c2sc(-c3ccc(C4CCN(C5CCOC5)CC4)c(F)c3)cc2Cl)C1] | ((S)-3-amino- pyrrolidin-1-yl) (3-chloro-5-(3- fluoro-4-(1- (tetrahydrofuran- 3-yl)piperidin- 4-yl)phenyl) thiophen-2-yl) methanone | ¹H NMR (400 MHz, MeOD) δ 7.53- 7.38 (m, 4H), 4.20 (d, J = 11.0 Hz, 1H), 4.09 (dd, J = 8.7, 4.2 Hz, 1H), 4.01 (d, J = 8.3 Hz, 3H), 3.89 (dd, J = 10.8, 6.1 Hz, 1H), 3.74 (ddd, J = 27.0, 18.0, 10.1 Hz, 5H), 3.58 (d, J = 11.9 Hz, 1H), 3.27 (s, 3H), 2.45 (dd, J = 13.6, 7.7 Hz, 2H), 2.27 (d, J = 8.3 Hz, 1H), 2.15 (d, J = 5.0 Hz, 5H). | 478.2 [M + H]⁺ |
| Compound 267 | | | |
| 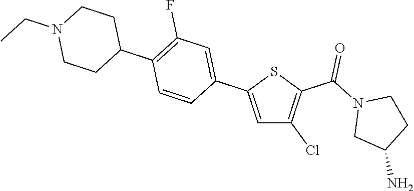 [SMILES: CCN1CCC(c2ccc(-c3cc(Cl)c(C(=O)N4CC[C@H](N)C4)s3)cc2F)CC1] | (S)-(3-amino- pyrrolidin-1-yl) (3-chloro-5-(4- (1-ethyl- piperidin-4-yl)- 3-fluorophenyl) thiophen-2-yl) methanone | ¹H NMR (400 MHz, MeOD) δ 7.51- 7.39 (m, 4H), 4.02 (d, J = 9.2 Hz, 2H), 3.89-3.66 (m, 5H), 3.29-3.20 (m, 3H), 3.20-3.09 (m, 2H), 2.47 (s, 1H), 2.24- 2.07 (m, 5H), 1.41 (t, J = 6.4 Hz, 3H). | 436.1 [M + H]⁺ |
| Compound 266 | | | |
| 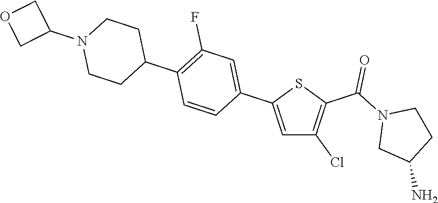 [SMILES: N[C@H]1CCN(C(=O)c2sc(-c3ccc(C4CCN(C5COC5)CC4)c(F)c3)cc2Cl)C1] | (S)-(3-amino- pyrrolidin-1-yl) (3-chloro-5-(3- fluoro-4-(1- (oxetan-3-yl) piperidin-4-yl) phenyl) thiophen-2-yl) methanone | ¹H NMR (400 MHz, MeOD) δ 7.47- 7.34 (m, 4H), 4.71 (t, J = 6.7 Hz, 2H), 4.63 (t, J = 6.3 Hz, 2H), 3.86-3.60 (m, 4H), 3.59-3.51 (m, 1H), 3.37 (s, 1H), 2.93 (d, J = 12.0 Hz, 3H), 2.22 (s, 1H), 2.02 (dd, J = 15.9, 10.2 Hz, 2H), 1.85 (dd, J = 8.5, 5.8 Hz, 5H). | 464.2 [M + H]⁺ |
| Compound 265 | | | |
| 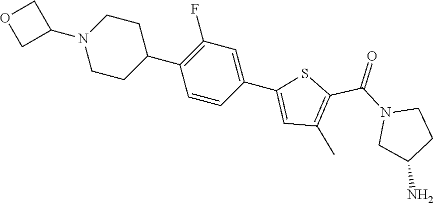 [SMILES: Cc1cc(-c2ccc(C3CCN(C4COC4)CC3)c(F)c2)sc1C(=O)N1CC[C@H](N)C1] | (S)-(3-amino- pyrrolidin-1-yl) (5-(3-fluoro-4- (1-(oxetan-3-yl) piperidin-4-yl) phenyl)-3- methylthiophen- 2-yl) methanone | ¹H NMR (400 MHz, MeOD) δ 7.41 (dd, J = 8.1, 1.6 Hz, 1H), 7.34 (dd, J = 14.0, 4.6 Hz, 2H), 7.24 (s, 1H), 4.71 (t, J = 6.7 Hz, 2H), 4.63 (t, J = 6.3 Hz, 2H), 3.75 (dd, J = 12.6, 5.9 Hz, 2H), 3.65- 3.58 (m, 2H), 3.54 (dd, J = 13.0, 6.6 Hz, 1H), 3.37-3.32 (m, 1H), 2.94-2.91 (m, 3H), 2.32 (s, 3H), 2.24-2.12 (m, 1H), 2.05-1.98 (m, 2H), 1.87-1.79 (m, 5H). | 444.1 [M + H]⁺ |
| Compound 264 | | | |
| 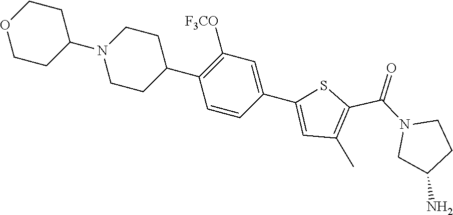 [SMILES: Cc1cc(-c2ccc(C3CCN(C4CCOCC4)CC3)c(OC(F)(F)F)c2)sc1C(=O)N1CC[C@H](N)C1] | (S)-(3-amino- pyrrolidin-1-yl) (3-methyl-5-(4- (1-(tetrahydro- 2H-pyran-4-yl) piperidin-4-yl)- 3-(trifluoro- methoxy) phenyl)thiophen- 2-yl)methanone | ¹H NMR (400 MHz, DMSO) δ 9.61 (s, 1H), 8.12 (s, 3H), 7.73-7.70 (m, 1H), 7.60 (s, 1H), 7.50 (s, 1H), 7.46 (d, J = 8.3 Hz, 1H), 4.01-3.99 (m, 2H), 3.76- 3.67 (m, 3H), 3.64-3.54 (m, 4H), 3.49- 3.43 (m, 1H), 3.34 (t, J = 11.4 Hz, 2H), 3.28-3.16 (m, 3H), 2.29 (s, 3H), 2.24 (dd, J = 14.1, 8.1 Hz, 1H), 2.04-1.97 (m, 7H), 1.75-1.67 (m, 2H). | 538.20 [M + H]⁺ |
| Compound 261 | | | |
| 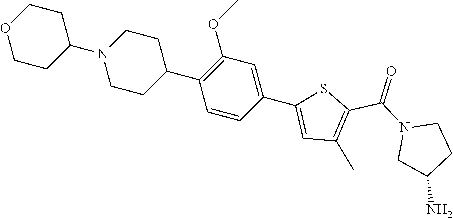 [SMILES: COc1cc(-c2cc(C)c(C(=O)N3CC[C@H](N)C3)s2)ccc1C1CCN(C2CCOCC2)CC1] | (S)-(3-amino- pyrrolidin-1-yl) (5-(3-methoxy-4- (1-(tetrahydro- 2H-pyran-4-yl) piperidin-4-yl) phenyl)-3- methylthiophen- 2-yl) methanone | ¹H NMR (400 MHz, MeOD) δ 7.28 (s, 1H), 7.22 (s, 2H), 7.20 (s, 1H), 4.11 (d, J = 4.3 Hz, 1H), 4.08 (d, J = 4.3 Hz, 1H), 4.00-3.97 (m, 2H), 3.91 (s, 3H), 3.86-3.75 (m, 2H), 3.72-3.66 (m, 3H), 3.49-3.44 (m, 3H), 3.19 (dd, J = 12.7, 10.2 Hz, 2H), 2.49-2.40 (m, 1H), 2.36 (s, 3H), 2.16-1.97 (m, 8H), 1.87-1.77 (m, 2H). | 484.35 [M + H]⁺ |
| Compound 260 | | | |
| 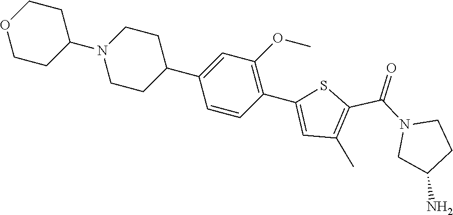 [SMILES: COc1cc(C2CCN(C3CCOCC3)CC2)ccc1-c1cc(C)c(C(=O)N2CC[C@H](N)C2)s1] | (S)-(3-amino- pyrrolidin-1-yl) (5-(2-methoxy- 4-(1-(tetrahydro- 2H-pyran-4-yl) piperidin-4-yl) phenyl)-3- methylthiophen- 2-yl) methanone | ¹H NMR (400 MHz, MeOD) δ 7.68 (d, J = 8.1 Hz, 1H), 7.37 (s, 1H), 6.99 (s, 1H), 6.93 (dd, J = 8.1, 1.4 Hz, 1H), 4.10 (dd, J = 11.5, 4.3 Hz, 2H), 4.02- 3.97 (m, 2H), 3.95 (s, 3H), 3.87-3.76 (m, 2H), 3.73-3.66 (m,, 3H), 3.52- 3.44 (m, 3H), 3.21-3.13 (m, 2H), 2.98- 2.92 (m, 1H), 2.50-2.39 (m, 1H), 2.35 (s, 3H), 2.20 (d, J = 14.2 Hz, 2H), 2.14-1.99 (m, 5H), 1.88-1.78 (m, 2H). | 484.40 [M + H]⁺ |
| Compound 259 | | | |

### Example 82: Synthesis of (S)-(3-aminopyrrolidin-1-yl)(5-(3-fluoro-4-(1-(tetrahydro-2H-pyran-4-yl)piperidin-4-yl)phenyl)-3-methylthiophen-2-yl)methanone (Compound 296)

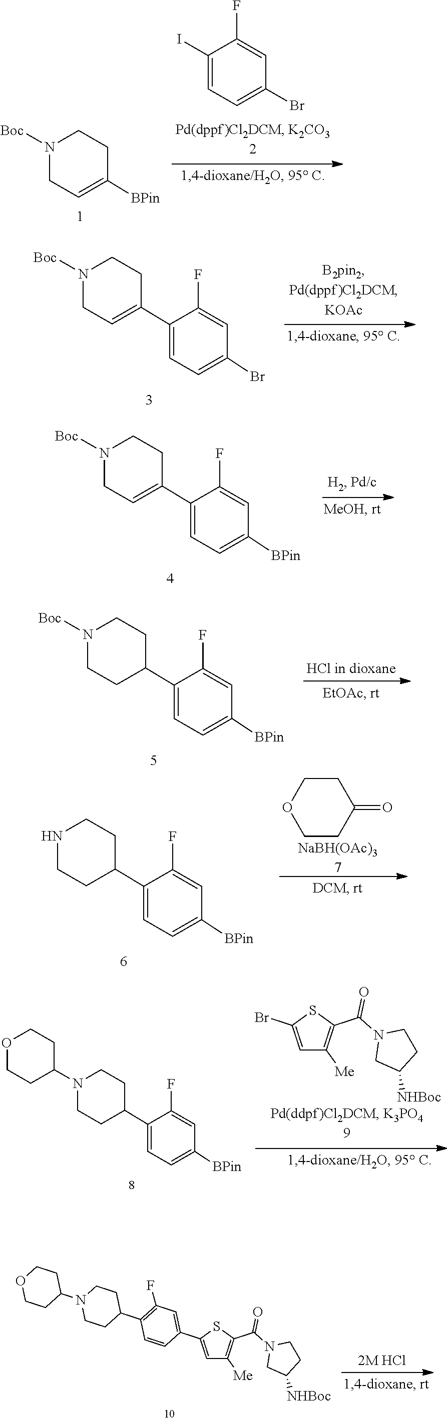

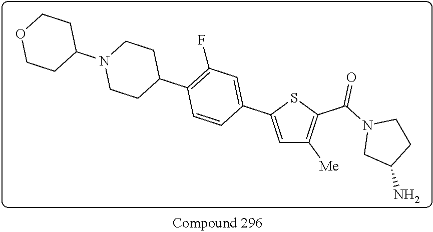 [SMILES: Cc1cc(-c2ccc(C3CCN(C4CCOCC4)CC3)c(F)c2)sc1C(=O)N1CC[C@H](N)C1]

### Step 1 Synthesis of tert-butyl 4-(4-bromo-2-fluorophenyl)-3,6-dihydropyridine-1(2H)-carboxylate

To a solution of 4-bromo-2-fluoro-1-iodobenzene (5 g, 16.6 mmol), tert-butyl 4-(4,4,5,5-tetramethyl-1,3,2-dioxaborolan-2-yl)-3,6-dihydropyridine-1(2H)-carboxylate (5.15 g, 16.6 mmol) and K₂CO₃ (6.87 g, 49.8 mmol) in dioxane/H₂O (45 mL, v/v=8/1) was added Pd(dppf)Cl₂DCM (1.35 g, 1.6 mmol), the mixture was stirred at 95° C. under N₂ for 16 h. After completion, the mixture was concentrated under reduced pressure and the residue was purified by flash column chromatography (PE/EA=10/1) to afford tert-butyl 4-(4-bromo-2-fluorophenyl)-3,6-dihydropyridine-1(2H)-carboxylate (3.7 g, 62.65% yield) as a yellow oil.

LCMS (ESI) calculated for C₁₆H₁₉BrFNO₂⁺ [M+2H-^{t}Bu]⁺ m/z 300.00, found 299.60.

### Step 2 Synthesis of tert-butyl 4-(2-fluoro-4-(4,4,5,5-tetramethyl-1,3,2-dioxaborolan-2-yl)phenyl)-3,6-dihydropyridine-1(2H)-carboxylate

To a solution of tert-butyl 4-(4-bromo-2-fluorophenyl)-3,6-dihydropyridine-1(2H)-carboxylate (3.7 g, 10.4 mmol), B₂Pin₂ (3.2 g, 12.4 mmol), and KOAc (2.0 g, 20.8 mmol) in dioxane (50 mL) was added Pd(dppf)Cl₂DCM (0.9 g, 1.0 mmol), the mixture was stirred at 95° C. under N₂ for 16 h. After completion, the mixture was concentrated under reduced pressure and the residue was purified by flash column chromatography (PE/EA=10/1) to afford tert-butyl 4-(2-fluoro-4-(4,4,5,5-tetramethyl-1,3,2-dioxaborolan-2-yl)phenyl)-3,6-dihydropyridine-1(2H)-carboxylate (4.3 g, 92.310% yield) as a yellow oil.

LCMS (ESI) calculated for C₂₂H₃₁BFNO₄⁺ [M+2H-^{t}Bu]⁺ m/z 348.17, found 348.05.

### Step 3 Synthesis of tert-butyl 4-(2-fluoro-4-(4,4,5,5-tetramethyl-1,3,2-dioxaborolan-2-yl)phenyl)piperidine-1-carboxylate

To a solution of tert-butyl 4-(2-fluoro-4-(4,4,5,5-tetramethyl-1,3,2-dioxaborolan-2-yl)phenyl)-3,6-dihydropyridine-1(2H)-carboxylate (4.4 g, 10.9 mmol) in MeOH (250 mL) was added Pd/C (0.46 g), the mixture was stirred at 25° C. under H₂ for 16 h. After completion, the mixture was filtered and concentrated under reduced pressure to afford tert-butyl 4-(2-fluoro-4-(4,4,5,5-tetramethyl-1,3,2-dioxaborolan-2-yl)phenyl)piperidine-1-carboxylate (3.73 g, 76.15% yield) as a yellow oil.

LCMS (ESI) calculated for C₁₆H₂₁FNO₂Na⁺ [M-^{t}Bu+H+Na]⁺ m/z 391.19, found 391.15.

### Step 4 Synthesis of 4-(2-fluoro-4-(4,4,5,5-tetramethyl-1,3,2-dioxaborolan-2-yl)phenyl)piperidine

To a solution of tert-butyl 4-(2-fluoro-4-(4,4,5,5-tetramethyl-1,3,2-dioxaborolan-2-yl)phenyl)piperidine-1-carboxylate (3.7 g, 9.1 mmol) in EtOAc (10 mL) was added HCl (20 mL, 2N in dioxane), the mixture was stirred at 25° C. for 16 hours. After completion, the solvent was removed under reduced pressure to afford 4-(2-fluoro-4-(4,4,5,5-tetramethyl-1,3,2-dioxaborolan-2-yl)phenyl)piperidine (3 g, 86.81% yield) as a yellow oil.

LCMS (ESI) calculated for C₁₇H₂₅BFNO₂⁺ [M+H]⁺ m/z 306.20, found 305.70.

### Step 5 Synthesis of 4-(2-fluoro-4-(4,4,5,5-tetramethyl-1,3,2-dioxaborolan-2-yl)phenyl)-1-(tetrahydro-2H-pyran-4-yl)piperidine

To a mixture of 4-(2-fluoro-4-(4,4,5,5-tetramethyl-1,3,2-dioxaborolan-2-yl)phenyl)piperidine (3 g, 9.8 mmol) and oxan-4-one (2.9 g, 29.4 mmol) in DCM (20 mL) was added STAB (6.2 g, 29.4 mmol), the mixture was stirred at 25° C. for 16 hours. After completion, the mixture was concentrated to dryness. The crude product was purified by column chromatography to afford 4-(2-fluoro-4-(4,4,5,5-tetramethyl-1,3,2-dioxaborolan-2-yl)phenyl)-1-(tetrahydro-2H-pyran-4-yl)piperidine (1.3 g, 47.96% yield) as a yellow solid.

LCMS (ESI) calculated for C₂₂H₃₃BFNO₃⁺ [M+H]⁺ m/z 390.25, found 389.85.

### Step 6 Synthesis of tert-butyl (S)-(1-(5-(3-fluoro-4-(1-(tetrahydro-2H-pyran-4-yl)piperidin-4-yl)phenyl)-3-methylthiophene-2-carbonyl)pyrrolidin-3-yl)carbamate

To a solution of 4-(2-fluoro-4-(4,4,5,5-tetramethyl-1,3,2-dioxaborolan-2-yl)phenyl)-1-(tetrahydro-2H-pyran-4-yl)piperidine (150 mg, 0.3853 mmol), tert-butyl (S)-(1-(5-bromo-3-methylthiophene-2-carbonyl)pyrrolidin-3-yl)carbamate (165 mg, 0.4238 mmol) and K₃PO₄ (164 mg, 0.7706 mmol) in 1,4-dioxane/H₂O (9 mL, v/v=8:1) was added Pd(dppf)Cl₂DCM (31 mg, 0.0385 mmol), the mixture was stirred at 95° C. under N₂ for 16 hours. After completion, the mixture was concentrated under reduced pressure and the residue was purified by column chromatography (DCM/MeOH=10/1) to afford tert-butyl (S)-(1-(5-(3-fluoro-4-(1-(tetrahydro-2H-pyran-4-yl)piperidin-4-yl)phenyl)-3-methylthiophene-2-carbonyl)pyrrolidin-3-yl)carbamate (120 mg, 51.75% yield) as a black oil.

LCMS (ESI) calcd for C₃₁H₄₃FN₃O₄S⁺ [M+H]⁺ m/z 572.30, found 572.40.

### Step 7 Synthesis of (S)-(3-aminopyrrolidin-1-yl)(5-(3-fluoro-4-(1-(tetrahydro-2H-pyran-4-yl)piperidin-4-yl)phenyl)-3-methylthiophen-2-yl)methanone (Compound 296)

To a solution of tert-butyl (S)-(1-(5-(3-fluoro-4-(1-(tetrahydro-2H-pyran-4-yl)piperidin-4-yl)phenyl)-3-methylthiophene-2-carbonyl)pyrrolidin-3-yl)carbamate (60 mg, 0.1049 mmol) in 1,4-dioxane (5 mL) was added HCl (5 mL, 2 N in 1,4-dioxane). The mixture was stirred at 25° C. for 2 hours. After completion, the solvent was removed under reduced pressure to afford (S)-(3-aminopyrrolidin-1-yl) (5-(3-fluoro-4-(1-(tetrahydro-2H-pyran-4-yl)piperidin-4-yl)phenyl)-3-methylthiophen-2-yl)methanone (40 mg, 78.93% yield) as a yellow solid.

LCMS (ESI) calcd for C₂₆H₃₅FN₃O₂S⁺ [M+H]⁺ m/z 472.24, found 472.15.

¹H NMR (400 MHz, MeOD) δ 7.48-7.46 (m, 1H), 7.39 (dd, J=17.2, 9.7 Hz, 2H), 7.30 (s, 1H), 4.10 (dd, J=11.6, 4.2 Hz, 2H), 4.00-3.97 (m, 2H), 3.88-3.76 (m, 2H), 3.74-3.68 (m, 3H), 3.28-3.19 (m, 3H), 3.22 (dd, J=15.6, 11.0 Hz, 3H), 2.49-2.43 (m, 1H), 2.35 (s, 3H), 2.18-2.09 (m, 7H), 1.89-1.81 (m, 2H).

| Compound | Name | ¹H NMR | MS |
|---|---|---|---|
| 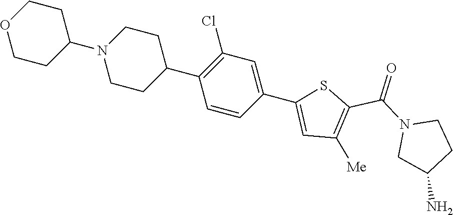 [SMILES: Cc1cc(-c2ccc(C3CCN(C4CCOCC4)CC3)c(Cl)c2)sc1C(=O)N1CC[C@H](N)C1] | (S)-(3-aminopyrrolidin- 1-yl)(5-(3-chloro-4-(1- (tetrahydro-2H-pyran- 4-yl)piperidin-4-yl) phenyl)-3-methyl- thiophen-2-yl) methanone | ¹H NMR (400 MHz, MeOD) δ 7.70 (s, 1H), 7.61 (d, J = 8.4 Hz, 1H), 7.43 (d, J = 8.4 Hz, 1H), 7.31 (s, 1H), 4.13-4.06 (m, 2H), 4.04-3.96 (m, 2H), 3.90- 3.80 (m, 1H), 3.79-3.69 (m, 4H), 3.54-3.41 (m, 4H), 3.25 (t, J = 11.6 Hz, 2H), 2.52-2.40 (m, 1H), 2.36 (s, 3H), 2.20-2.10 (m, 7H), 1.93-1.81 (m, 2H). | 488.3 [M + H]⁺ |
| Compound 287 | | | |
| 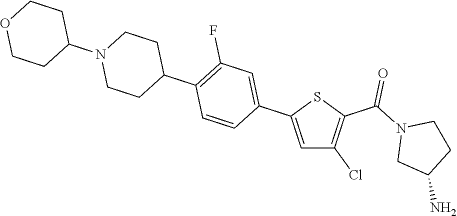 [SMILES: N[C@H]1CCN(C(=O)c2sc(-c3ccc(C4CCN(C5CCOCC5)CC4)c(F)c3)cc2Cl)C1] | (S)-(3-aminopyrrolidin- 1-yl)(3-chloro-5-(3- fluoro-4-(1-(tetrahydro- 2H-pyran-4-yl) piperidin-4-yl)phenyl) thiophen-2-yl) methanone | ¹H NMR (400 MHz, DMSO) δ 10.90 (s, 1H), 8.42 (s, 3H), 7.67 (s, 1H), 7.63 (dd, J = 11.5, 1.5 Hz, 1H), 7.55 (dd, J = 8.0, 1.4 Hz, 1H), 7.38 (t, J = 8.0 Hz, 1H), 4.02-3.95 (m, 2H), 3.92-3.79 (m, 2H), 3.78-3.63 (m, 4H), 3.45-3.27 (m, 4H), 3.21-3.05 (m, 3H), 2.33-2.18 (m, 3H), 2.10-2.03 (m, 3H), 2.01-1.92 (m, 2H), 1.83-1.70 (m, 2H). | 492.2 [M + H]⁺ |
| Compound 295 | | | |
| 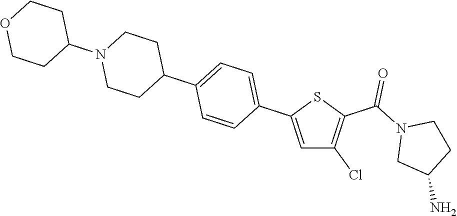 [SMILES: N[C@H]1CCN(C(=O)c2sc(-c3ccc(C4CCN(C5CCOCC5)CC4)cc3)cc2Cl)C1] | (S)-(3-aminopyrrolidin- 1-yl)(3- chloro-5-(4-(1-(tetra- hydro-2H-pyran-4-yl) piperidin-4-yl)phenyl) thiophen-2-yl) methanone | ¹H NMR (400 MHz, MeOD) δ 7.63 (d, J = 7.6 Hz, 2H), 7.36 (t, J = 8.0 Hz, 3H), 4.18-4.07 (m, 2H), 4.02 (d, J = 8.5 Hz, 2H), 3.90-3.77 (m, 2H), 3.74-3.65 (m, 3H), 3.62-3.53 (m, 1H), 3.48 (t, J = 11.6 Hz, 2H), 3.26- 3.10 (m, 2H), 2.98 (t, J = 11.7 Hz, 1H), 2.53-2.41 (m, 1H), 2.25-2.03 (m, 7H), 1.97-1.77 (m, 2H). | 474.2 [M + H]⁺ |
| Compound 294 | | | |
| 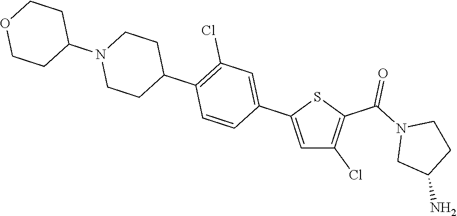 [SMILES: N[C@H]1CCN(C(=O)c2sc(-c3ccc(C4CCN(C5CCOCC5)CC4)c(Cl)c3)cc2Cl)C1] | (S)-(3-aminopyrrolidin- 1-yl)(3-chloro-5-(3- chloro-4-(1- (tetrahydro-2H-pyran- 4-yl)piperidin-4-yl) phenyl)thiophen-2- yl)methanone | ¹H NMR (400 MHz, MeOD) δ 7.74 (s, 1H), 7.63 (d, J = 7.6 Hz, 1H), 7.47 (d, J = 7.6 Hz, 1H), 7.43 (s, 1H), 4.09 (d, J = 8.8 Hz, 2H), 4.02 (d, J = 8.8 Hz, 2H), 3.90-3.78 (m, 2H), 3.77- 3.69 (m, 3H), 3.48 (t, J = 11.2 Hz, 4H), 3.26 (s, 2H), 2.47 (s, 1H), 2.15 (d, J = 15.6 Hz, 7H), 1.94-1.81 (m, 2H). | 508.2 [M + H]⁺ |
| Compound 273 | | | |

### Example 83: Synthesis of (S)-(3-aminopyrrolidin-1-yl)(5-(3-fluoro-4-(1-(tetrahydro-2H-pyran-4-yl)piperidin-4-yl)phenyl)-3-methoxythiophen-2-yl)methanone (Compound 292)

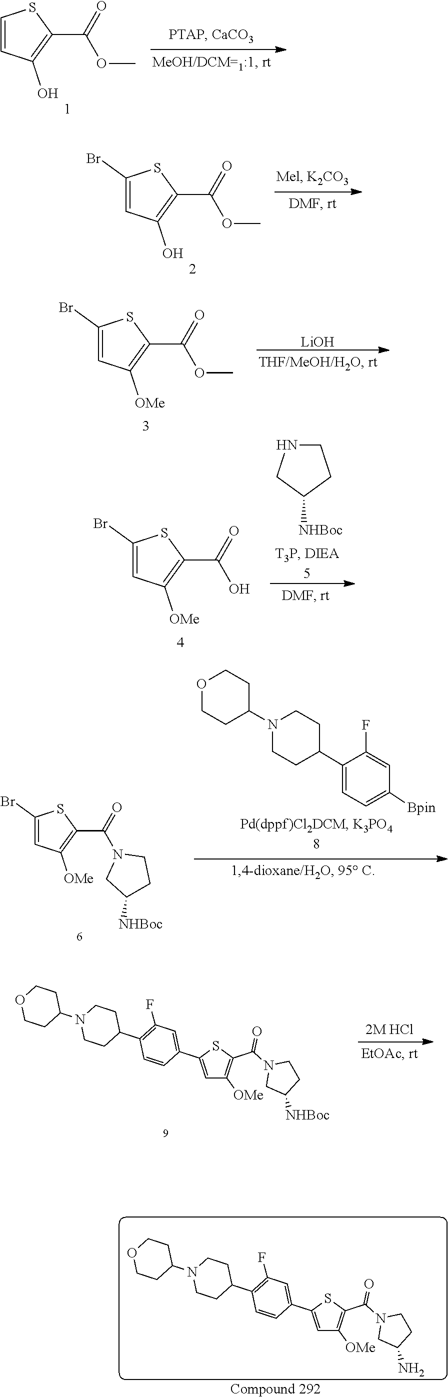

### Step 1 Synthesis of 5-bromo-3-hydroxythiophene-2-carboxylate

To a solution of methyl 3-hydroxythiophene-2-carboxylate (3 g, 19 mmol) in DCM/MeOH (60 mL, v/v=1/1) was added phenyltrimethylammonium tribromide (21.43 g, 57 mmol) and calcium carbonate (7.76 g, 76 mmol), the mixture was stirred at 25° C. for 16 hours. After completion, the mixture was filtered through celite, and the filtrate was concentrated. The residue was diluted with water and extracted with EtOAc (100 mL*3). The combined organic layers were washed with brine, dried over sodium sulfate, filtered, and concentrated under vacuum. The residue was purified by flash column chromatography using petroleum ether/EtOAc=1/1 as eluent to afford methyl 5-bromo-3-hydroxythiophene-2-carboxylate (1 g, 21.05% yield) as a colorless oil.

¹H NMR (400 MHz, DMSO) δ 10.37 (s, 1H), 7.98 (s, 1H), 3.81 (s, 3H).

### Step 2 Synthesis of methyl 5-bromo-3-methoxythiophene-2-carboxylate

To a solution of methyl 5-bromo-3-hydroxythiophene-2-carboxylate (1 g, 4.2 mmol) in DMF (10 mL) was added Mel (0.72 g, 5.04 mol) and K₂CO₃ (1.45 g, 10.5 mmol), the mixture was stirred at 25° C. for 6 hours. After completion, the mixture was diluted with H₂O and the resulted mixture was extracted with EtOAc for 3 times. The combined organic phases were washed with brine, dried over Na₂SO₄ and filtered. The solvent was removed under reduced pressure and the residue was purified by flash column chromatography using petroleum ether/EtOAc=20/1 as eluent to afford methyl 5-bromo-3-methoxythiophene-2-carboxylate (0.8 g, 71.43% yield) as a yellow oil.

¹H NMR (400 MHz, DMSO) δ 8.05 (s, 1H), 3.91 (s, 3H), 3.82 (s, 3H).

### Step 3 Synthesis of 5-bromo-3-methoxythiophene-2-carboxylic acid

To a solution of methyl 5-bromo-3-methoxythiophene-2-carboxylate (800 mg, 3.186 mmol) in MeOH/THF/H₂O (18 mL, v/v/v=1/1/1) was added LiOH (381.49 mg, 15.93 mmol), the mixture was stirred at 25° C. for 6 hours. After completion, the solvent was removed under reduced pressure. Then 1M HCl in water was added to adjust pH to 3~4. The mixture was extracted with EtOAc (30 mL) for 3 times. The organic layers were washed with brine, dried over Na₂SO₄ and filtered, the solvent was removed under reduced pressure to afford 5-bromo-3-methoxythiophene-2-carboxylic acid (700 mg, 83.41% yield) as a white solid.

LCMS (ESI) calcd for C₆H₆BrO₃S⁺ [M+H]⁺ m/z 236.92, found 236.9.

### Step 4 Synthesis of tert-butyl (S)-(1-(5-bromo-3-methoxythiophene-2-carbonyl)pyrrolidin-3-yl)carbamate

To a solution of 5-bromo-3-methoxythiophene-2-carboxylic acid (700 mg, 2.9527 mmol), tert-butyl (S)-pyrrolidin-3-ylcarbamate (550 mg, 2.9527 mmol) and DIEA (1526 mg, 11.8108 mmol) in DMF (10 mL) was added T3P (2818 mg, 8.8581 mmol). The mixture was stirred at 25° C. for 2 hours. After completion, the solvent was removed and the residue was purified by flash column chromatography using DCM/MeOH=10/1 as eluent to afford tert-butyl (S)-(1-(5-bromo-3-methoxythiophene-2-carbonyl)pyrrolidin-3-yl)carbamate (790 mg, 62.71% yield) as a yellow oil.

LCMS (ESI) calculated for C₁₅H₂₂BrN₂O₄S⁺ [M+H]⁺ m/z 405.05, found 405.0.

### Step 5 Synthesis of tert-butyl (S)-(1-(5-(3-fluoro-4-(1-(tetrahydro-2H-pyran-4-yl)piperidin-4-yl)phenyl)-3-methoxythiophene-2-carbonyl)pyrrolidin-3-yl)carbamate

To a solution of 4-(2-fluoro-4-(4,4,5,5-tetramethyl-1,3,2-dioxaborolan-2-yl)phenyl)-1-(tetrahydro-2H-pyran-4-yl)piperidine (200 mg, 0.5137 mmol), tert-butyl (S)-(1-(5-bromo-3-methoxythiophene-2-carbonyl)pyrrolidin-3-yl)carbamate (208 mg, 0.5137 mmol), and K₃PO₄ (327 mg, 1.5411 mmol) in 1,4-dioxane/H₂O (9 mL, v/v=8/1) was added Pd(dppf)Cl₂DCM (42 mg, 0.05137 mmol). The mixture was stirred at 95° C. for 16 hours. After completion, the solvent was removed under reduced pressure and the residue was purified by column chromatography using dichloromethane/methanol=10/1 as eluent to afford tert-butyl (S)-(1-(5-(3-fluoro-4-(1-(tetrahydro-2H-pyran-4-yl)piperidin-4-yl)phenyl)-3-methoxythiophene-2-carbonyl)pyrrolidin-3-yl)carbamate (230 mg, 68.56% yield) as a dark oil.

LCMS (ESI) calcd for C₃₁H₄₂FN₃O₅S⁺ [M+H]⁺ m/z 588.29, found 588.6.

### Step 6 Synthesis of (S)-(3-aminopyrrolidin-1-yl)(5-(3-fluoro-4-(1-(tetrahydro-2H-pyran-4-yl)piperidin-4-yl)phenyl)-3-methoxythiophen-2-yl)methanone (Compound 292)

To a solution of tert-butyl (S)-(1-(5-(3-fluoro-4-(1-(tetrahydro-2H-pyran-4-yl)piperidin-4-yl)phenyl)-3-methoxythiophene-2-carbonyl)pyrrolidin-3-yl)carbamate (230 mg, 0.3913 mmol) in EtOAc (10 mL) was added HCl (5 mL, 2N in EtOAc), the mixture was stirred at 25° C. for 2 hours. After completion, the mixture was concentrated under reduced pressure and the residue was purified by prep-HPLC to afford (S)-(3-aminopyrrolidin-1-yl)(5-(3-fluoro-4-(1-(tetrahydro-2H-pyran-4-yl)piperidin-4-yl)phenyl)-3-methoxythiophen-2-yl)methanone (47.5 mg, 23.64%) as a white solid.

LCMS (ESI) calculated for C₂₆H₃₄FN₃O₃S [M+H]⁺ m/z 488.23, found 488.1.

¹H NMR (400 MHz, MeOD) δ 7.61 (s, 1H), 7.40 (dd, J=8.1, 1.4 Hz, 1H), 7.33 (dd, J=9.3, 4.5 Hz, 2H), 4.01 (dd, J=11.2, 3.9 Hz, 2H), 3.81 (dd, J=11.8, 6.1 Hz, 2H), 3.75 (s, 3H), 3.71-3.55 (m, 2H), 3.42 (t, J=11.2 Hz, 2H), 3.16 (d, J=11.6 Hz, 2H), 2.90 (dt, J=10.0, 5.8 Hz, 1H), 2.60-2.50 (m, 1H), 2.35 (td, J=11.3, 3.4 Hz, 2H), 2.25-2.13 (m, 1H), 1.85 (dd, J=16.1, 9.7 Hz, 7H), 1.60 (ddd, J=24.4, 12.3, 4.4 Hz, 3H).

The following compounds were prepared analogously:

| Compound | Name | ¹H NMR | MS |
|---|---|---|---|
| 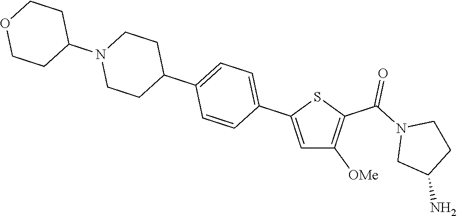 [SMILES: COc1cc(-c2ccc(C3CCN(C4CCOCC4)CC3)cc2)sc1C(=O)N1CC[C@H](N)C1] | (S)-3-aminopyrrolidin- 1-yl)(3-methoxy-5-(4- (1-(tetrahydro-2H- pyran-4-yl)piperidin-4- yl)phenyl)thiophen-2- yl)methanone | ¹H NMR (400 MHz, MeOD) δ 7.54 (d, J = 8.5 Hz, 3H), 7.29 (d, J = 8.2 Hz, 2H), 4.01 (dd, J = 11.1, 4.2 Hz, 2H), 3.80 (d, J = 5.8 Hz, 2H), 3.71 (s, 3H), 3.61 (dd, J = 32.2, 6.3 Hz, 2H), 3.42 (t, J = 11.2 Hz, 3H), 3.16 (d, J = 11.5 Hz, 2H), 2.64-2.52 (m, 2H), 2.35 (t, J = 10.8 Hz, 2H), 2.26-2.13 (m, 1H), 1.95- 1.75 (m, 7H), 1.62 (tt, J = 12.3, 6.3 Hz, 2H). | 470.5 [M + H]⁺ |
| Compound 293 | | | |
| 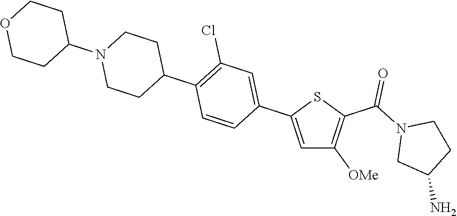 [SMILES: COc1cc(-c2ccc(C3CCN(C4CCOCC4)CC3)c(Cl)c2)sc1C(=O)N1CC[C@H](N)C1] | (S)-(3-aminopyrrolidin- 1-yl)(5-(3-chloro-4-(1- (tetrahydro-2H-pyran- 4-yl)piperidin-4-yl) phenyl)-3-methoxy- thiophen-2-yl) methanone | ¹H NMR (400 MHz, MeOD) δ 7.72 (d, J = 3.8 Hz, 2H), 7.61 (d, J = 7.9 Hz, 1H), 7.44 (d, J = 7.8 Hz, 1H), 4.10 (d, J = 8.0 Hz, 2H), 4.03 (s, 2H), 3.85 (s, 2H), 3.74 (s, 6H), 3.48 (t, J = 11.6 Hz, 4H), 3.26 (s, 2H), 2.47 (s, 1H), 2.16 (dd, J = 31.9, 12.4 Hz, 7H), 1.87 (d, J = 8.7 Hz, 2H). | 504.2 [M + H]⁺ |
| Compound 272 | | | |

### Example 84: Synthesis of (S)-(3-aminopyrrolidin-1-yl)(3-ethyl-5-(3-fluoro-4-(1-(tetrahydro-2H-pyran-4-yl)piperidin-4-yl)phenyl)thiophen-2-yl)methanone (Compound 291)

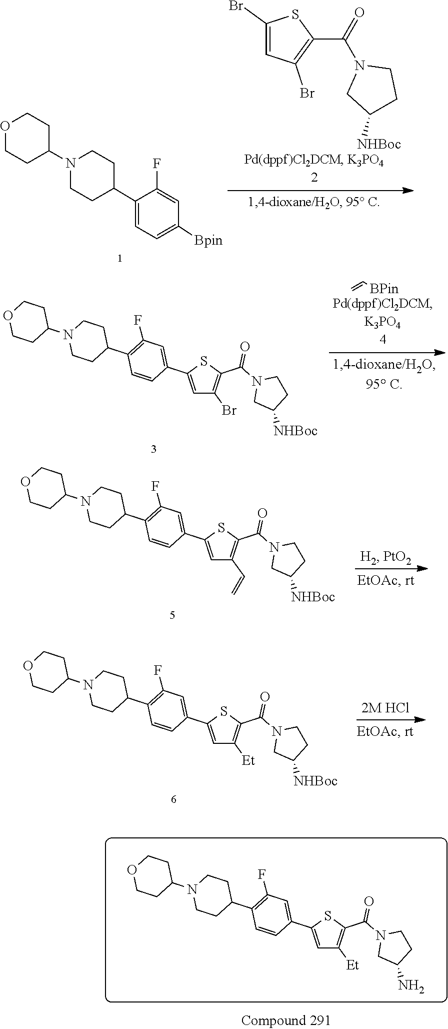

### Step 1 Synthesis of tert-butyl (S)-(1-(3-bromo-5-(3-fluoro-4-(1-(tetrahydro-2H-pyran-4-yl)piperidin-4-yl)phenyl)thiophene-2-carbonyl)pyrrolidin-3-yl)carbamate

To a solution of 4-(2-fluoro-4-(4,4,5,5-tetramethyl-1,3,2-dioxaborolan-2-yl)phenyl)-1-(tetrahydro-2H-pyran-4-yl)piperidine (500 mg, 1.8026 mmol), tert-butyl (S)-(1-(3,5-dibromothiophene-2-carbonyl)pyrrolidin-3-yl)carbamate (982 mg, 2.1631 mmol) and K₃PO₄ (765 mg, 3.6052 mmol) in 1,4-dioxane/H₂O (20 mL, v/v=8/1) was added Pd(dppf)Cl₂DCM (147 mg, 0.1802 mmol), the mixture was stirred at 95° C. under N₂ for 16 h. After completion, the mixture was concentrated and the residue was purified by flash column chromatography to afford tert-butyl (S)-(1-(3-bromo-5-(3-fluoro-4-(1-(tetrahydro-2H-pyran-4-yl)piperidin-4-yl)phenyl)thiophene-2-carbonyl)pyrrolidin-3-yl)carbamate (1 g, 87.14% yield) as a yellow oil.

LCMS (ESI) calculated for C₃₀H₃₉BrFN3O₄S⁺ [M+H]⁺ m/z 638.18, found 637.95.

### Step 2 Synthesis of tert-butyl (S)-(1-(5-(3-fluoro-4-(1-(tetrahydro-2H-pyran-4-yl)piperidin-4-yl)phenyl)-3-vinylthiophene-2-carbonyl)pyrrolidin-3-yl)carbamate

To a solution of tert-butyl (S)-(1-(3-bromo-5-(3-fluoro-4-(1-(tetrahydro-2H-pyran-4-yl)piperidin-4-yl)phenyl)thiophene-2-carbonyl)pyrrolidin-3-yl)carbamate (1 g, 1.6 mmol), 4,4,5,5-tetramethyl-2-vinyl-1,3,2-dioxaborolane (0.3 g, 1.9 mmol) and K₃PO₄ (0.68 g, 3.2 mmol) in 1,4-dioxane/H₂O (18 mL, v/v=8/1) was added Pd(dppf)Cl₂DCM (0.13 g, 0.16 mmol), the mixture was stirred at 95° C. under N₂ for 16 h. After completion, the mixture was concentrated and the residue was purified by flash column chromatography to afford tert-butyl (S)-(1-(5-(3-fluoro-4-(1-(tetrahydro-2H-pyran-4-yl)piperidin-4-yl)phenyl)-3-vinylthiophene-2-carbonyl)pyrrolidin-3-yl)carbamate (0.94 g, 81.25% yield) as a dark oil.

LCMS (ESI) calculated for C₃₂H₄₂FN₃O₄S⁺ [M+H]⁺ m/z 584.29, found 584.35.

### Step 3 Synthesis of tert-butyl (S)-(1-(3-ethyl-5-(3-fluoro-4-(1-(tetrahydro-2H-pyran-4-yl)piperidin-4-yl)phenyl)thiophene-2-carbonyl)pyrrolidin-3-yl)carbamate

To a solution of tert-butyl (S)-(1-(5-(3-fluoro-4-(1-(tetrahydro-2H-pyran-4-yl)piperidin-4-yl)phenyl)-3-vinylthiophene-2-carbonyl)pyrrolidin-3-yl)carbamate (600 mg, 1.0278 mmol) in EtOAc (100 mL) was added PtO₂ (70 mg, 0.3083 mmol). The mixture was stirred at 25° C. under H₂ for 16 h. After completion, the mixture was filtered. The filtrate was concentrated and the residue was purified by flash column chromatography using DCM/MeOH=10/1 as eluent to afford tert-butyl (S)-(1-(3-ethyl-5-(3-fluoro-4-(1-(tetrahydro-2H-pyran-4-yl)piperidin-4-yl)phenyl)thiophene-2-carbonyl)pyrrolidin-3-yl)carbamate (200 mg, 33.22% yield) as a yellow oil.

LCMS (ESI) calculated for C₃₂H₄₄FN₃O₄S⁺ [M+H]⁺ m/z 586.30, found 586.30.

### Step 4 Synthesis of (S)-(3-aminopyrrolidin-1-yl)(3-ethyl-5-(3-fluoro-4-(1-(tetrahydro-2H-pyran-4-yl)piperidin-4-yl)phenyl)thiophen-2-yl)methanone (Compound 291)

To a solution of tert-butyl (S)-(1-(3-ethyl-5-(3-fluoro-4-(1-(tetrahydro-2H-pyran-4-yl)piperidin-4-yl)phenyl)thiophene-2-carbonyl)pyrrolidin-3-yl)carbamate (200 mg, 0.3414 mmol) in EtOAc (10 mL) was added HCl (5 mL, 2N in 1,4-dioxane), the mixture was stirred at 25° C. for 16 hours. After completion, the solvent was removed under reduced pressure to afford (S)-(3-aminopyrrolidin-1-yl)(3-ethyl-5-(3-fluoro-4-(1-(tetrahydro-2H-pyran-4-yl)piperidin-4-yl)phenyl)thiophen-2-yl)methanone (130 mg, 78.41% yield) as a yellow solid.

LCMS (ESI) calculated for C₂₇H₃₆FN₃O₂S⁺ [M+H]⁺ m/z 486.25, found 486.25.

¹H NMR (400 MHz, MeOD) δ 7.47 (d, J=7.7 Hz, 1H), 7.44-7.36 (m, 3H), 4.12-4.06 (m, 2H), 4.04-3.95 (m, 2H), 3.82 (t, J=9.1 Hz, 1H), 3.78-3.66 (m, 4H), 3.48 (t, J=11.7 Hz, 3H), 3.24 (d, J=11.3 Hz, 3H), 2.73 (q, J=7.4 Hz, 2H), 2.45 (d, J=6.9 Hz, 1H), 2.16 (t, J=21.2 Hz, 7H), 1.87 (d, J=8.8 Hz, 2H), 1.27 (t, J=7.4 Hz, 3H).

| Compound | Name | ¹H NMR | MS |
|---|---|---|---|
| 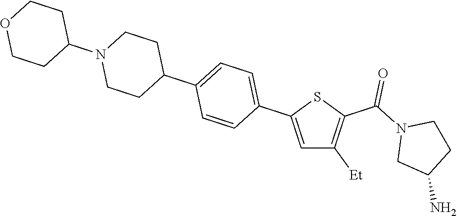 [SMILES: CCc1cc(-c2ccc(C3CCN(C4CCOCC4)CC3)cc2)sc1C(=O)N1CC[C@H](N)C1] | (S)-(3-aminopyrrolidin- 1-yl)(3-ethyl-5-(4-(1- (tetrahydro-2H-pyran- 4-yl)piperidin-4-yl) phenyl)thiophen-2- yl)methanone | ¹H NMR (400 MHz, MeOD) δ 7.63 (d, J = 6.8 Hz, 2H), 7.41- 7.25 (m, 3H), 4.09 (d, J = 9.5 Hz, 2H), 4.01 (s, 2H), 3.82 (s, 1H), 3.70 (s, 4H), 3.48 (t, J = 11.0 Hz, 3H), 3.20 (s, 2H), 2.97 (s, 1H), 2.74 (q, J = 7.2 Hz, 2H), 2.44 (s, 1H), 2.15 (s, 7H), 1.88 (s, 2H), 1.27 (t, J = 7.2 Hz, 3H). | 468.2 [M + H]⁺ |
| Compound 289 | | | |
| 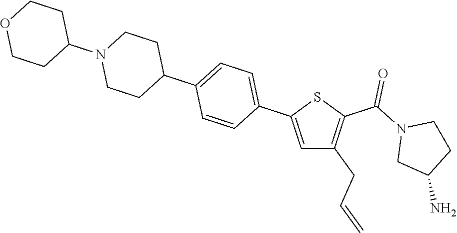 [SMILES: C=CCc1cc(-c2ccc(C3CCN(C4CCOCC4)CC3)cc2)sc1C(=O)N1CC[C@H](N)C1] | (S)-(3-allyl-5-(4-(1- (tetrahydro-2H-pyran- 4-yl)piperidin-4-yl) phenyl)thiophen-2-yl) (3-aminopyridin-1-yl) methanone | ¹H NMR (400 MHz, MeOD) δ 7.62 (d, J = 6.5 Hz, 2H), 7.35 (d, J = 6.5 Hz, 2H), 7.26 (s, 1H), 6.00 (td, J = 15.8, 5.9 Hz, 1H), 5.15 (d, J = 17.0 Hz, 1H), 5.09 (d, J = 9.9 Hz, 1H), 4.10 (d, J = 8.7 Hz, 2H), 4.00 (s, 2H), 3.82 (s, 1H), 3.71 (s, 4H), 3.50 (d, J = 6.5 Hz, 5H), 3.20 (s, 2H), 2.97 (s, 1H), 2.42 (d, J = 12.3 Hz, 1H), 2.14 (d, J = 6.0 Hz, 7H), 1.87 (s, 2H). | 480.6 [M + H]⁺ |
| Compound 286 | | | |
| 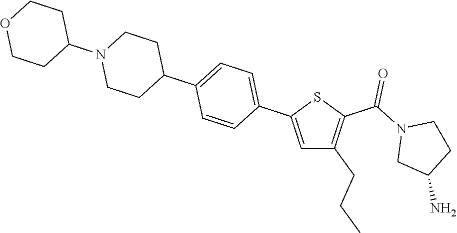 [SMILES: CCCc1cc(-c2ccc(C3CCN(C4CCOCC4)CC3)cc2)sc1C(=O)N1CC[C@H](N)C1] | (S)-(3-aminopyrrolidin- 1-yl)(3-propyl-5-(4-(1- (tetrahydro-2H-pyran- 4-yl)piperidin-4-yl) phenyl)thiophen-2- yl)methanone | ¹H NMR (400 MHz, MeOD) δ 7.63 (d, J = 8.0 Hz, 2H), 7.34 (d, J = 8.0 Hz, 2H), 7.30 (s, 1H), 4.10 (dd, J = 11.3, 3.9 Hz, 2H), 4.04-3.95 (m, 2H), 3.87- 3.80 (m, 1H), 3.72 (td, J = 18.1, 8.0 Hz, 4H), 3.47 (t, J = 11.4 Hz, 3H), 3.20 (t, J = 11.7 Hz, 2H), 2.96 (t, J = 12.5 Hz, 1H), 2.74-2.65 (m, 2H), 2.44 (d, J = 6.7 Hz, 1H), 2.14 (dd, J = 28.6, 12.1 Hz, 7H), 1.92-1.80 (m, 2H), 1.75-1.65 (m, 2H), 0.97 (t, J = 7.3 Hz, 3H). | 482.4 [M + H]⁺ |
| Compound 285 | | | |

### Example 85: Synthesis of (S)-(3-aminopyrrolidin-1-yl)(3-methyl-5-(4-(1-(2-(2-(prop-2-yn-1-yloxy)ethoxy)ethyl)piperidin-4-yl)phenyl)thiophen-2-yl)methanone (Compound 290)

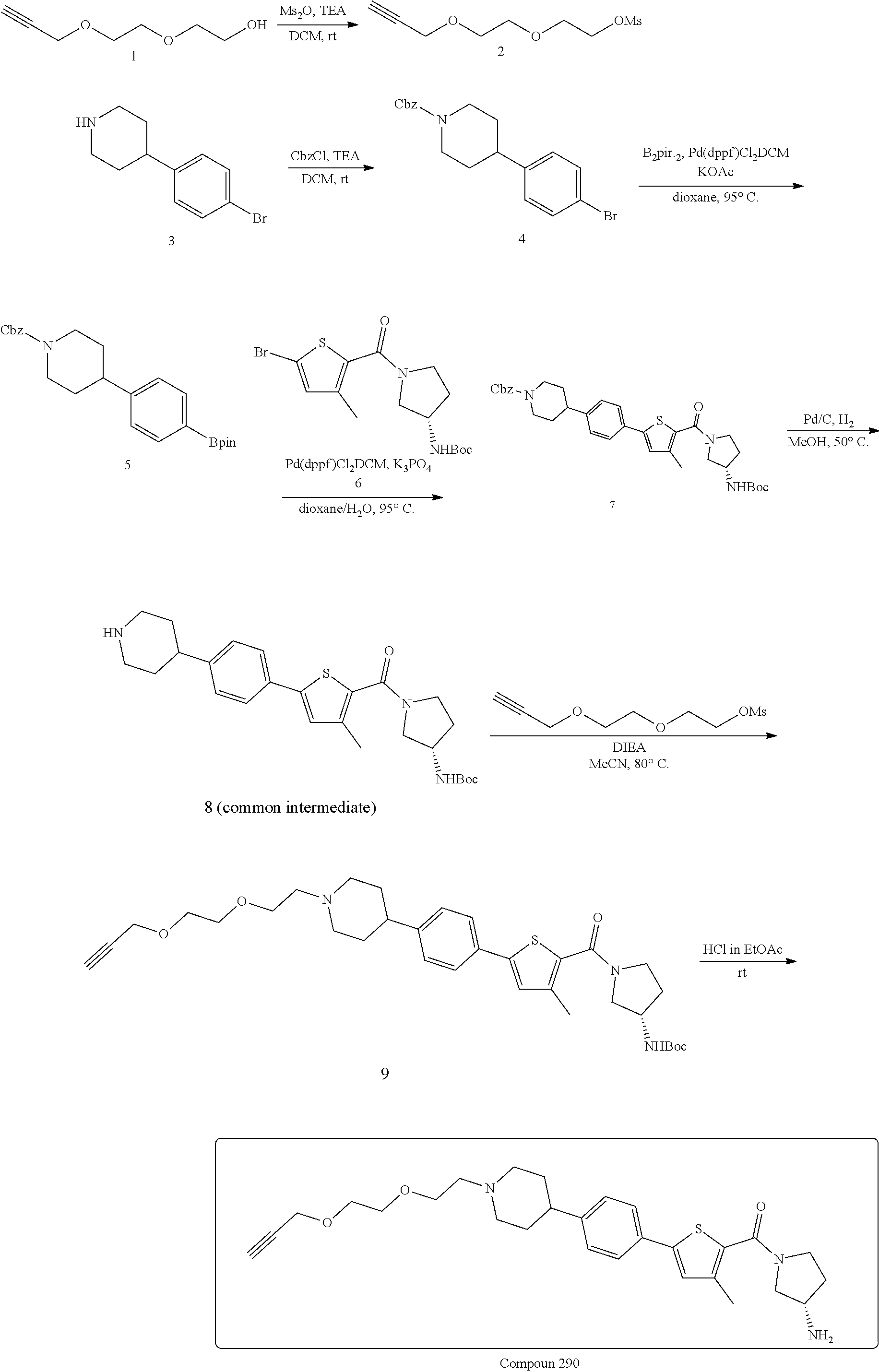

### Step 1 Synthesis of 2-(2-(prop-2-yn-1-yloxy)ethoxy)ethyl methanesulfonate

To a solution of 2-(2-(prop-2-yn-1-yloxy)ethoxy)ethan-1-o (250 mg, 1.7341 mmol) and triethylamine (263 mg, 2.6011 mmol) in DCM (10 mL) was added Ms₂O (332 mg, 1.9075 mmol) at 0° C. The mixture was stirred at 25° C. for 1 h. After completion, the mixture was concentrated and the residue was purified by column chromatography using petroleum ether/ethyl acetate=2/1 as eluent to afford 2-(2-(prop-2-yn-1-yloxy)ethoxy)ethyl methanesulfonate (200 mg, 49.30% yield) as a colorless oil.

¹H NMR (400 MHz, DMSO) δ 4.32-4.30 (m, 2H), 4.15 (d, J=2.4 Hz, 2H), 3.68-3.66 (m, 2H), 3.60-3.55 (m, 4H), 3.42 (t, J=2.4 Hz, 1H), 3.18 (s, 3H).

### Step 2 Synthesis of benzyl 4-(4-bromophenyl)piperidine-1-carboxylate

To a solution of 4-(4-bromophenyl)piperidine (2 g, 8.3 mmol) and triethylamine (1.26 g, 12.4 mmol) in DCM (30 mL) was added benzyl carbonochloridate (1.56 g, 9.1 mmol) at 0° C. The mixture was stirred at 25° C. for 16 hours. After completion, the mixture was concentrated and the residue was purified by column chromatography using petroleum ether/ethyl acetate=2/1 as eluent to afford benzyl 4-(4-bromophenyl)piperidine-1-carboxylate (2.2 g, 67.47% yield) as a yellowish oil.

LCMS (ESI) calcd for C₁₉H₂₁BrNO2 [M+H]⁺ m/z 376.07, found 376.05.

### Step 3 Synthesis of benzyl 4-(4-(4,4,5,5-tetramethyl-1,3,2-dioxaborolan-2-yl)phenyl)piperidine-1-carboxylate

To a solution of benzyl 4-(4-bromophenyl)piperidine-1-carboxylate (2.4 g, 6.4 mmol), 4,4,5,5-tetramethyl-2-(4,4,5,5-tetramethyl-1,3,2-dioxaborolan-2-yl)-1,3,2-dioxaborolane (2.44 g, 9.6 mmol) and KOAc (1.26 g, 12.8 mmol) in 1,4-dioxane (30 mL) was added Pd(dppf)Cl₂DCM (0.52 g, 0.64 mmol), the mixture was stirred at 95° C. under N₂ for 16 h. After completion, the mixture was concentrated and the residue was purified by column chromatography using petroleum ether/ethyl acetate=1/1 as eluent to afford benzyl 4-(4-(4,4,5,5-tetramethyl-1,3,2-dioxaborolan-2-yl)phenyl)piperidine-1-carboxylate (2.2 g, 78.13% yield) as a yellowish oil.

LCMS (ESI) calcd for C₂₅H₃₃BNO4⁺ [M+H] m/z 422.25, found 422.10.

### Step 4 Synthesis of benzyl (S)-4-(4-(5-(3-((tert-butoxycarbonyl)amino)pyrrolidine-1-carbonyl)-4-methylthiophen-2-yl)phenyl)piperidine-1-carboxylate

To a solution of benzyl 4-(4-(4,4,5,5-tetramethyl-1,3,2-dioxaborolan-2-yl)phenyl)piperidine-1-carboxylate (2.5 g, 5.9 mmol), tert-butyl (S)-(1-(5-bromo-3-methylthiophene-2-carbonyl)pyrrolidin-3-yl)carbamate (2.3 g, 5.9 mmol) and K₃PO₄ (3.13 g, 14.7 mmol) in 1,4-dioxane/1H₂O (27 mL, v/v=8/1) was added Pd(dppf)Cl₂DCM (0.48 g, 0.59 mmol), the mixture was stirred at 95° C. under N₂ for 16 h. After completion, the mixture was concentrated and the residue was purified by column chromatography using petroleum ether/ethyl acetate=1/1 as eluent to afford benzyl (S)-4-(4-(5-(3-((tert-butoxycarbonyl)amino)pyrrolidine-1-carbonyl)-4-methylthiophen-2-yl)phenyl)piperidine-1-carboxylate (1.6 g, 42.37% yield) as a yellowish oil.

LCMS (ESI) calcd for C₃₄H₄₂N3O₅S [M+H]⁺ m/z 604.28, found 604.15.

### Step 5 Synthesis of tert-butyl (S)-(1-(3-methyl-5-(4-(piperidin-4-yl)phenyl)thiophene-2-carbonyl)pyrrolidin-3-yl)carbamate

To a solution of benzyl (S)-4-(4-(5-(3-((tert-butoxycarbonyl)amino)pyrrolidine-1-carbonyl)-4-methylthiophen-2-yl)phenyl)piperidine-1-carboxylate (1.3 g, 2.2 mmol) in MeOH (50 mL) was added Pd/C (0.13 g). The mixture was stirred at 50° C. under H₂ atmosphere for 48 hours. After completion, the mixture was filtered. The filtrate was concentrated and the residue was purified by column chromatography using DCM/MeOH=5/1 as eluent to afford tert-butyl (S)-(1-(3-methyl-5-(4-(piperidin-4-yl)phenyl)thiophene-2-carbonyl)pyrrolidin-3-yl)carbamate (0.6 g, 54.55% yield) as a yellowish oil.

LCMS (ESI) calcd for C₂₆H₃₆N3O₃S [M+H]⁺ m/z 470.25, found 470.50.

### Step 6 Synthesis of tert-butyl (S)-(1-(3-methyl-5-(4-(1-(2-(2-(prop-2-yn-1-yloxy)ethoxy)ethyl)piperidin-4-yl)phenyl)thiophene-2-carbonyl)pyrrolidin-3-yl)carbamate

To a solution of tert-butyl (S)-(1-(3-methyl-5-(4-(piperidin-4-yl)phenyl)thiophene-2-carbonyl)pyrrolidin-3-yl)carbamate (200 mg, 0.4259 mmol) and 2-(prop-2-yn-1-yloxy)ethyl methanesulfonate (91 mg, 0.5110 mmol) in MeCN (10 mL) was added DIEA (165 mg, 1.2777 mmol), the mixture was stirred at 80° C. for 16 hours. After completion, the mixture was concentrated and the residue was purified by column chromatography using DCM/MeOH=5/1 as eluent to afford tert-butyl (S)-(1-(3-methyl-5-(4-(1-(2-(prop-2-yn-1-yloxy)ethyl)piperidin-4-yl)phenyl)thiophene-2-carbonyl)pyrrolidin-3-yl)carbamate (150 mg, 60.65% yield) as a white solid.

LCMS (ESI) calcd for C₃₁H₄₂N3O₄S⁺ [M+H]⁺ m/z 552.29, found 552.35.

### Step 7 Synthesis of (S)-(3-aminopyrrolidin-1-yl)(3-methyl-5-(4-(1-(2-(2-(prop-2-yn-1-yloxy)ethoxy)ethyl)piperidin-4-yl)phenyl)thiophen-2-yl)methanone (Compound 290)

To a solution of tert-butyl (S)-(1-(3-methyl-5-(4-(1-(2-(2-(prop-2-yn-1-yloxy)ethoxy)ethyl)piperidin-4-yl)phenyl)thiophene-2-carbonyl)pyrrolidin-3-yl)carbamate (180 mg, 0.3021 mmol) in EtOAc (8 mL) was added HCl (4 mL, 2N in EtOAc), the mixture was stirred at 25° C. for 16 hours. After completion, the solvent was removed under reduced pressure to afford (S)-(3-aminopyrrolidin-1-yl)(3-methyl-5-(4-(1-(2-(2-(prop-2-yn-1-yloxy)ethoxy)ethyl)piperidin-4-yl)phenyl)thiophen-2-yl)methanone (120 mg, 75.01% yield) as a yellow solid.

LCMS (ESI) calcd for C₂₈H₃₈N3O₃S⁺ [M+H]⁺ m/z 496.26, found 496.15.

¹H NMR (400 MHz, MeOD) δ 7.62 (d, J=8.1 Hz, 2H), 7.34 (d, J=8.2 Hz, 2H), 7.25 (s, 1H), 4.22 (dd, J=5.4, 2.4 Hz, 2H), 4.01-3.97 (m, 2H), 3.90-3.88 (m, 2H), 3.86-3.78 (m, 3H), 3.77-3.75 (m, 1H), 3.74-3.72 (m, 4H), 3.71-3.68 (m, 1H), 3.41-3.39 (m, 2H), 3.21 (t, J=11.4 Hz, 2H), 2.99-2.96 (m, 1H), 2.92 (t, J=2.3 Hz, 1H), 2.53-2.41 (m, 1H), 2.36 (s, 3H), 2.16-2.13 (m, 3H), 2.07-2.02 (m, 2H).

| Compound | Name | ¹H NMR | MS |
|---|---|---|---|
| 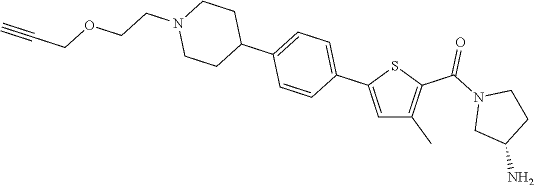 [SMILES: C#CCOCCN1CCC(c2ccc(-c3cc(C)c(C(=O)N4CC[C@H](N)C4)s3)cc2)CC1] Compound 288 | (S)-(3- amino- pyrrolidin-1- yl)(3-methyl- 5-(4-(1-(2- (prop-2-yn- 1-yloxy) ethyl) piperidin-4- yl)phenyl) thiophen-2- yl) methanone | ¹H NMR (400 MHz, MeOD) δ 7.62 (d, J = 8.1 Hz, 2H), 7.34 (d, J = 8.1 Hz, 2H), 7.25 (s, 1H), 4.30 (d, J = 2.3 Hz, 2H), 4.01-3.97 (m, 2H), 3.94-3.92 (m, 2H), 3.88-3.83 (m, 1H), 3.81-3.67 (m, 4H), 3.47-3.42 (m, 2H), 3.18 (dd, J = 25.2, 13.2 Hz, 2H), 3.01 (t, J = 2.2 Hz, 1H), 2.98-2.92 (m, 1H), 2.45 (dd, J = 13.6, 6.6 Hz, 1H), 2.36 (s, 3H), 2.16-2.13 (m, 3H), 2.07-2.04 (m, 2H). | 452.15 [M + H]⁺ |

### Example 86: Synthesis of (S)-(3-aminopyrrolidin-1-yl)(5-(3-fluoro-4-(1-(2-hydroxy-2-methylpropyl)piperidin-4-yl)phenyl)-3-methylthiophen-2-yl)methanone (Compound 257)

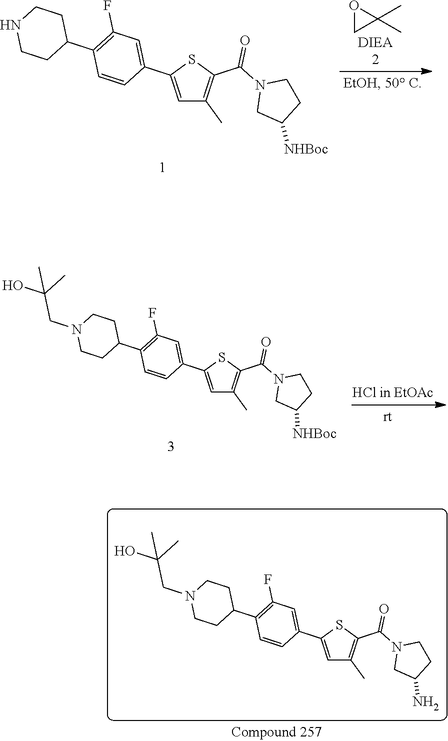

### Step 1 Synthesis of tert-butyl (S)-(1-(5-(3-fluoro-4-(1-(2-hydroxy-2-methylpropyl)piperidin-4-yl)phenyl)-3-methylthiophene-2-carbonyl)pyrrolidin-3-yl)carbamate

To a solution of tert-butyl (S)-(1-(5-(3-fluoro-4-(piperidin-4-yl)phenyl)-3-methylthiophene-2-carbonyl)pyrrolidin-3-yl)carbamate (200 mg, 0.4101 mmol) in EtOH (5 mL) were added DIEA (106 mg, 0.8202 mmol) and 2,2-dimethyl oxirane (30 mg, 0.4101 mmol), the mixture was stirred at 50° C. for 16 hours. After completion, the solvent was removed under reduced pressure and the residue was purified by flash column chromatography using petroleum ether/EtOAc=1/1 as eluent to afford tert-butyl (S)-(1-(5-(3-fluoro-4-(1-(2-hydroxy-2-methylpropyl)piperidin-4-yl)phenyl)-3-methylthiophene-2-carbonyl)pyrrolidin-3-yl)carbamate (90 mg, 35.28% yield) as a yellow oil.

LCMS (ESI) calculated for C₃₀H₄₃FN₃O₄S⁺ [M+H]⁺ m/z 560.29, found 560.3.

### Step 2 Synthesis of (S)-(3-aminopyrrolidin-1-yl)(5-(3-fluoro-4-(1-(2-hydroxy-2-methylpropyl)piperidin-4-yl)phenyl)-3-methylthiophen-2-yl)methanone (Compound 257)

To a solution of tert-butyl (S)-(1-(5-(3-fluoro-4-(1-(2-hydroxy-2-methylpropyl)piperidin-4-yl)phenyl)-3-methylthiophene-2-carbonyl)pyrrolidin-3-yl)carbamate (90 mg, 0.1608 mmol) in EtOAc (5 mL) was added HCl (5 mL, 2N in EtOAc), the mixture was stirred at 25° C. for 16 hours. After completion, the mixture was concentrated under reduced pressure to afford (S)-(3-aminopyrrolidin-1-yl)(5-(3-fluoro-4-(1-(2-hydroxy-2-methylpropyl)piperidin-4-yl)phenyl)-3-methylthiophen-2-yl)methanone (67 mg, 89.24% yield) as a yellow solid.

LCMS (ESI) calculated for C₂₅H₃₅FN₃O₂S⁺ [M+H]⁺ m/z 460.24, found 460.2.

1H NMR (400 MHz, MeOD) δ 7.50-7.37 (m, 3H), 7.30 (s, 1H), 4.00 (d, J=9.2 Hz, 2H), 3.92-3.66 (m, 5H), 3.49 (dd, J=33.4, 12.6 Hz, 1H), 3.29-3.18 (m, 4H), 2.45 (d, J=6.2 Hz, 1H), 2.36 (s, 3H), 2.13 (ddd, J=35.2, 23.1, 12.5 Hz, 6H), 1.39 (s, 6H).

| Compound | Name | ¹H NMR | MS |
|---|---|---|---|
| 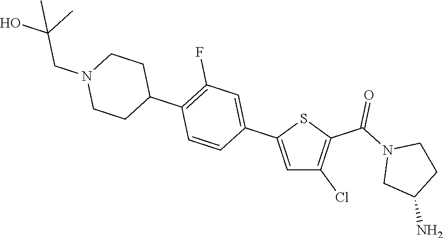 [SMILES: CC(C)(O)CN1CCC(c2ccc(-c3cc(Cl)c(C(=O)N4CC[C@H](N)C4)s3)cc2F)CC1] | (S)-(3- aminopyrrolidin-1- yl)(3-chloro-5-(3-fluoro- 4-(1-(2-hydroxy-2- methylpropyl) piperidin-4-yl)phenyl) thiophen-2-yl) methanone | ¹H NMR (400 MHz, MeOD) δ 7.54-7.39 (m, 4H), 4.07-3.96 (m, 2H), 3.93-3.65 (m, 5H), 3.50 (dd, J = 33.8, 12.8 Hz, 1H), 3.24 (d, J = 17.4 Hz, 4H), 2.47 (d, J = 6.6 Hz, 1H), 2.34-2.12 (m, 3H), 2.10-1.99 (m, 2H), 1.39 (d, J = 2.1 Hz, 6H). | 480.1 [M + H]⁺ |
| Compound 256 | | | |

### Example 87: Synthesis of (S)-(3-aminopyrrolidin-1-yl)(5-(3-fluoro-4-(1-(2-methoxy-2-methylpropyl)piperidin-4-yl)phenyl)-3-methylthiophen-2-yl)methanone (Compound 254)

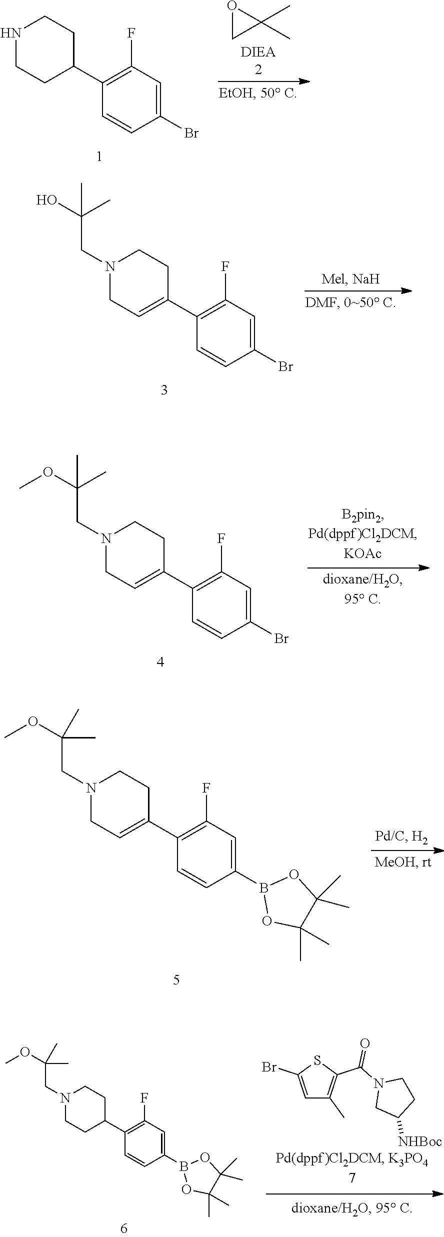

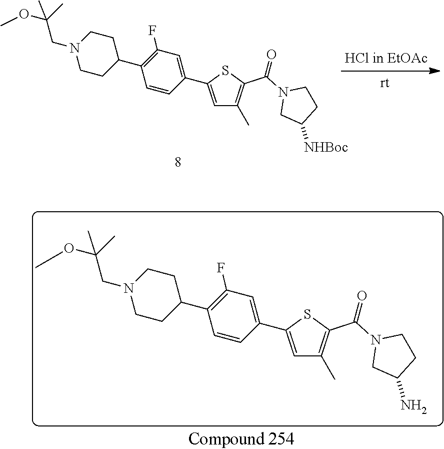 [SMILES: COC(C)(C)CN1CCC(c2ccc(-c3cc(C)c(C(=O)N4CC[C@H](NC(=O)OC(C)(C)C)C4)s3)cc2F)CC1 ; COC(C)(C)CN1CCC(c2ccc(-c3cc(C)c(C(=O)N4CC[C@H](N)C4)s3)cc2F)CC1]

### Step 1 Synthesis of 1-(4-(4-bromo-2-fluorophenyl)-3,6-dihydropyridin-1(2H)-yl)-2-methylpropan-2-ol

To a solution of 4-(4-bromo-2-fluorophenyl)-1,2,3,6-tetrahydropyridine (2 g, 7.8 mmol) in EtOH (10 mL) were added DIEA (2.02 g, 15.6 mmol) and 2,2-dimethyloxirane (0.67 g, 9.35 mmol), the mixture was stirred at 50° C. for 16 hours. After completion, the solvent was removed under reduced pressure and the residue was purified by flash column chromatography using petroleum ether/EtOAc=3/1 as eluent to afford 1-(4-(4-bromo-2-fluorophenyl)-3,6-dihydropyridin-1(2H)-yl)-2-methylpropan-2-ol (1.7 g, 60.26% yield) as a yellow oil.

LCMS (ESI) calculated for C₁₅H₂₀BrFNO⁺ [M+H]⁺ m/z 330.1, found 330.1.

### Step 2 Synthesis of 4-(4-bromo-2-fluorophenyl)-1-(2-methoxy-2-methylpropyl)-1,2,3,6-tetrahydropyridine

To a solution of 1-(4-(4-bromo-2-fluorophenyl)-3,6-dihydropyridin-1(2H)-yl)-2-methylpropan-2-ol (1.7 g, 5.2 mmol) in THF (10 mL) was added sodium hydride (0.19 g, 7.8 mmol, 60%), the mixture was stirred at 0° C. for 0.5 h. Then, Mel (0.81 g, 5.72 mmol) was added dropwise, the resulting mixture was stirred at 50° C. for 6 hours. After completion, the mixture was diluted with H₂O (100 mL) and extracted with EtOAc for 3 times. The combined organic layers were washed with brine, dried over Na₂SO₄, filtered, and concentrated. The residue was purified by flash column chromatography using petroleum ether/EtOAc=3/1 as eluent to afford 4-(4-bromo-2-fluorophenyl)-1-(2-methoxy-2-methylpropyl)-1,2,3,6-tetrahydropyridine (1.4 g, 71.15% yield) as a yellow oil.

LCMS (ESI) calculated for C₁₆H₂₂BrFNO⁺ [M+H]⁺ m/z 344.1, found 344.1.

### Step 3 Synthesis of 4-(2-fluoro-4-(4,4,5,5-tetramethyl-1,3,2-dioxaborolan-2-yl)phenyl)-1-(2-methoxy-2-methylpropyl)-1,2,3,6-tetrahydropyridine

To a solution of 4-(4-bromo-2-fluorophenyl)-1-(2-methoxy-2-methylpropyl)-1,2,3,6-tetrahydropyridine (1.4 g, 4.1 mmol), B₂Pin₂ (1.15 g, 4.51 mmol), and KOAc (1.21 g, 12.3 mmol) in 1,4-dioxane (10 mL) was added Pd(dppf)Cl₂DCM (0.33 g, 0.41 mmol), the mixture was stirred at 95° C. for 16 hours. After completion, the solvent was removed under reduced pressure and the residue was purified by column chromatography using petroleum ether/EtOAc=1/2 as eluent to afford 4-(2-fluoro-4-(4,4,5,5-tetramethyl-1,3,2-dioxaborolan-2-yl)phenyl)-1-(2-methoxy-2-methylpropyl)-1,2,3,6-tetrahydropyridine (1.1 g, 56.1% yield) as a yellow oil. LCMS (ESI) calcd for C₂₂H₃₄BFNO₃⁺ [M+H]⁺ m/z 390.26, found 390.2.

### Step 4 Synthesis of 4-(2-fluoro-4-(4,4,5,5-tetramethyl-1,3,2-dioxaborolan-2-yl)phenyl)-1-(2-methoxy-2-methylpropyl)piperidine

To a solution of 4-(2-fluoro-4-(4,4,5,5-tetramethyl-1,3,2-dioxaborolan-2-yl)phenyl)-1-(2-methoxy-2-methylpropyl)-1,2,3,6-tetrahydropyridine (1.1 g, 2.8 mmol) in MeOH (40 mL) was added 10% Pd/C (0.12 g), the mixture was stirred at room temperature under H₂ for 16 hours. After completion, the mixture was filtered through celite. The filtrate was concentrated under vacuum to afford 4-(2-fluoro-4-(4,4,5,5-tetramethyl-1,3,2-dioxaborolan-2-yl)phenyl)-1-(2-methoxy-2-methylpropyl)piperidine (0.9 g, 82.1% yield) as a yellow oil.

LCMS (ESI) calculated for C₂₂H₃₆BFNO₃⁺ [M+H]⁺ m/z 392.27, found 392.3.

### Step 5 Synthesis of tert-butyl (S)-(1-(5-(3-fluoro-4-(1-(2-methoxy-2-methylpropyl)piperidin-4-yl)phenyl)-3-methylthiophene-2-carbonyl)pyrrolidin-3-yl)carbamate

To a solution of 4-(2-fluoro-4-(4,4,5,5-tetramethyl-1,3,2-dioxaborolan-2-yl)phenyl)-1-(2-methoxy-2-methylpropyl)piperidine (200 mg, 0.5111 mmol), tert-butyl (S)-(1-(5-bromo-3-methylthiophene-2-carbonyl)pyrrolidin-3-yl)carbamate (199 mg, 0.5111 mmol), and K₃PO₄ (326 mg, 1.5333 mmol) in 1,4-dioxane/H₂O (10 mL, v/v=8/1) was added Pd(dppf)Cl₂DCM (42 mg, 0.05111 mmol), the mixture was stirred at 95° C. for 16 hours. After completion, the solvent was removed under reduced pressure and the residue was purified by column chromatography using dichloromethane/MeOH=10/1 as eluent to afford tert-butyl (S)-(1-(5-(3-fluoro-4-(1-(2-methoxy-2-methylpropyl)piperidin-4-yl)phenyl)-3-methylthiophene-2-carbonyl)pyrrolidin-3-yl)carbamate (50 mg, 16.2% yield) as a dark oil.

LCMS (ESI) calculated for C₃₁H₄₅FN₃O₄S⁺ [M+H]⁺ m/z 574.3, found 574.3.

### Step 6 Synthesis of (S)-(3-aminopyrrolidin-1-yl)(5-(3-fluoro-4-(1-(2-methoxy-2-methylpropyl)piperidin-4-yl)phenyl)-3-methylthiophen-2-yl)methanone (Compound 254)

To a solution of tert-butyl (S)-(1-(5-(3-fluoro-4-(1-(2-methoxy-2-methylpropyl)piperidin-4-yl)phenyl)-3-methylthiophene-2-carbonyl)pyrrolidin-3-yl)carbamate (50 mg, 0.0871 mmol) in EtOAc (5 mL) was added HCl (3 mL, 2N in EtOAc), the mixture was stirred at 25° C. for 16 hours. After completion, the mixture was concentrated under reduced pressure to afford (S)-(3-aminopyrrolidin-1-yl)(5-(3-fluoro-4-(1-(2-methoxy-2-methylpropyl)piperidin-4-yl)phenyl)-3-methylthiophen-2-yl)methanone (26.1 mg, 62.34% yield) as a yellow solid.

LCMS (ESI) calculated for C₂₆H₃₇FN₃O₂S⁺ [M+H]⁺ m/z 474.2, found 474.2.

¹H NMR (400 MHz, DMSO) δ 9.61 (s, 1H), 8.55 (s, 3H), 7.55-7.43 (m, 3H), 7.38 (t, J=8.0 Hz, 1H), 3.87-3.53 (m, 7H), 3.22 (d, J=4.7 Hz, 2H), 3.19 (s, 3H), 3.11 (d, J=12.7 Hz, 1H), 2.38 (dd, J=25.2, 12.7 Hz, 3H), 2.29 (s, 3H), 2.23 (d, J=6.6 Hz, 1H), 2.07 (d, J=5.4 Hz, 2H), 1.87 (d, J=11.9 Hz, 2H), 1.30 (s, 6H).

The following compounds were prepared analogously:

| Compound | Name | ¹H NMR | MS |
|---|---|---|---|
| 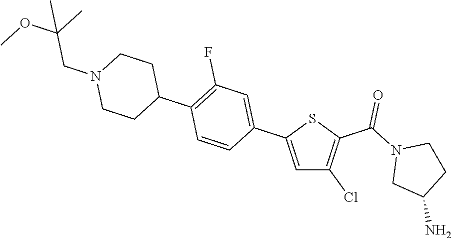 [SMILES: COC(C)(C)CN1CCC(c2ccc(-c3cc(Cl)c(C(=O)N4CC[C@H](N)C4)s3)cc2F)CC1] | (S)-(3-amino- pyrrolidin-1-yl)(3- chloro-5-(3-fluoro- 4-(1-(2- methoxy-2-methyl- propyl)piperidin- 4-yl)phenyl) thiophen-2- yl)methanone | ¹H NMR (400 MHz, DMSO) δ 9.67 (s, 1H), 8.58 (s, 3H), 7.71- 7.54 (m, 3H), 7.41 (t, J = 8.0 Hz, 1H), 3.81 (d, J = 11.5 Hz, 2H), 3.67 (d, J = 11.9 Hz, 3H), 3.58 (dd, J = 15.3, 9.8 Hz, 2H), 3.33 (s, 1H), 3.22 (d, J = 4.7 Hz, 3H), 3.19 (s, 3H), 3.12 (d, J = 12.3 Hz, 1H), 2.46- 2.32 (m, 2H), 2.31-2.22 (m, 1H), 2.09 (d, J = 5.2 Hz, 1H), 1.87 (d, J = 12.2 Hz, 2H), 1.30 (s, 6H). | 494.2 [M + H]⁺ |
| Compound 249 | | | |
| 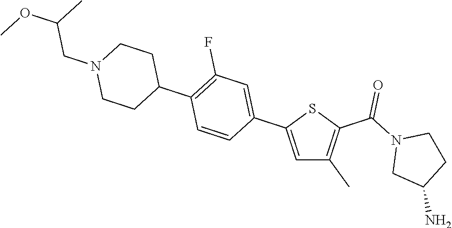 [SMILES: COC(C)CN1CCC(c2ccc(-c3cc(C)c(C(=O)N4CC[C@H](N)C4)s3)cc2F)CC1] | ((S)-3-amino- pyrrolidin-1-yl)(5- (3-fluoro-4-(1-(2- methoxypropyl) piperidin-4-yl) phenyl)-3- methylthiophen-2- yl)methanone | ¹H NMR (400 MHz, MeOD) δ 7.47 (d, J = 8.2 Hz, 1H), 7.39 (dd, J = 14.9, 9.7 Hz, 2H), 7.30 (s, 1H), 4.05- 3.97 (m, 2H), 3.92 (s, 1H), 3.86- 3.66 (m, 5H), 3.43 (s, 3H), 3.28-3.11 (m, 5H), 2.45 (dd, J = 13.1, 6.2 Hz, 1H), 2.35 (s, 3H), 2.14 (dt, J = 27.7, 14.5 Hz, 5H), 1.23 (d, J = 5.9 Hz, 3H). | 460.2 [M + H]⁺ |
| Compound 251 | | | |
| 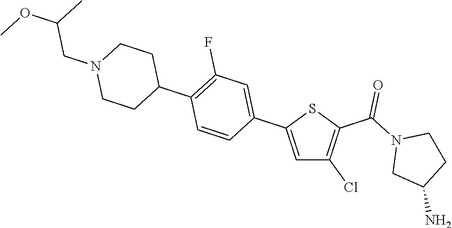 [SMILES: COC(C)CN1CCC(c2ccc(-c3cc(Cl)c(C(=O)N4CC[C@H](N)C4)s3)cc2F)CC1] | ((S)-3-amino- pyrrolidin-1-yl)(3- chloro-5-(3-fluoro- 4-(1-(2- methoxypropyl) piperidin-4-yl) phenyl)thiophen- 2-yl)methanone | ¹H NMR (400 MHz, MeOD) δ 7.57- 7.35 (m, 4H), 4.02 (d, J = 9.0 Hz, 2H), 3.91 (d, J = 5.7 Hz, 1H), 3.77 (ddd, J = 27.1, 14.6, 7.4 Hz, 5H), 3.43 (s, 3H), 3.29-3.09 (m, 5H), 2.47 (d, J = 6.7 Hz, 1H), 2.15 (dt, J = 27.0, 15.2 Hz, 5H), 1.23 (d, J = 5.9 Hz, 3H). | 480.2 [M + H]⁺ |
| Compound 250 | | | |

### Example 88: Synthesis of (S)-(3-aminopyrrolidin-1-yl)(5-(3-chloro-4-(1-(tetrahydro-2H-pyran-4-yl)piperidin-4-yl)phenyl)-3-(trifluoromethoxy)thiophen-2-yl)methanone (Compound 252)

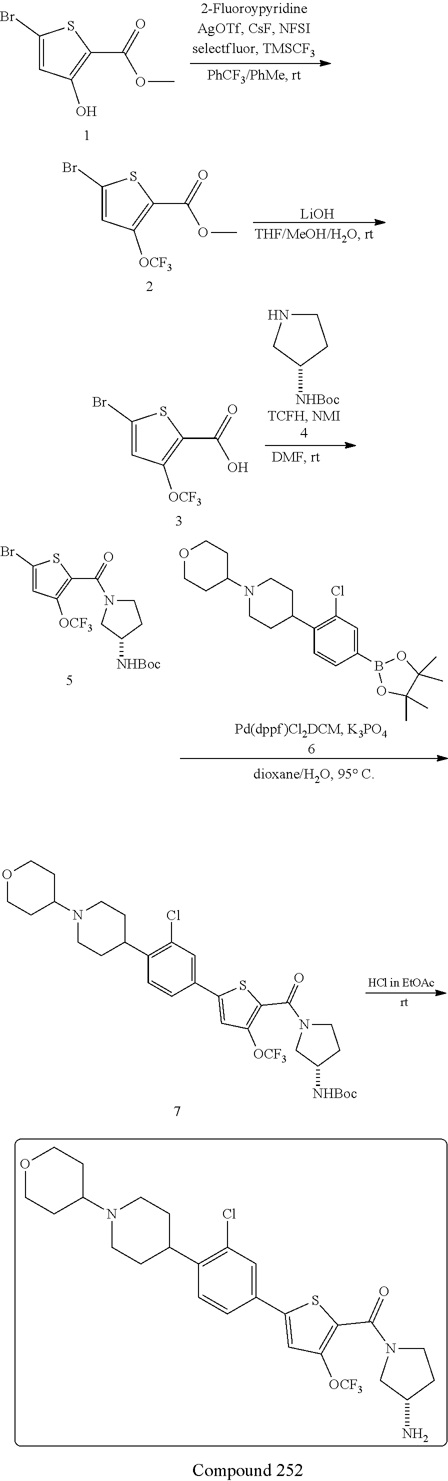

### Step 1 Synthesis of methyl 5-bromo-3-(trifluoromethoxy)thiophene-2-carboxylate

To a solution of methyl 5-bromo-3-hydroxythiophene-2-carboxylate (4.8 g, 0.0202 mol), AgOTf (25.95 g, 0.101 mol), Selectfluor (14.31 g, 0.0404 mol), NFSI (12.74 g, 0.0404 mmol), and CsF (18.42 g, 0.1212 mol) in toluene/benzotrifluoride (60 mL, v/v=1/2) were added 2-fluoropyridine (9.81 g, 0.101 mol, 5.0 equiv) and TMSCF3 (14.36 g, 0.101 mol, 5.0 equiv), the reaction mixture was stirred at room temperature under N2 for 16 h. After completion, the reaction mixture was filtered through a plug of silica (eluted with Ethyl acetate). The filtrate was concentrated, and the product was purified by column chromatography on silica gel to afford methyl 5-bromo-3-(trifluoromethoxy)thiophene-2-carboxylate (650 mg, 10.4% yield) as a colorless oil.

¹H NMR (400 MHz, DMSO) δ 8.27 (s, 1H), 3.86 (s, 3H).

### Step 2 Synthesis of 5-bromo-3-(trifluoromethoxy)thiophene-2-carboxylic acid

To a solution of methyl 5-bromo-3-(trifluoromethoxy)thiophene-2-carboxylate (150 mg, 0.4589 mmol) in MeOH/THF/H₂O (6 mL, v/v/v=1/1/1) was added LiOH (55 mg, 2.2945 mmol), the mixture was stirred at 25° C. for 6 hours. After completion, the solvent was removed under reduced pressure. Then 1 M HCl in water was added to adjust pH to 3~4. The mixture was extracted with EtOAc (30 mL*3). The combined organic layers were washed with brine, dried over Na₂SO₄, and filtered. The solvent was removed under reduced pressure to afford 5-bromo-3-(trifluoromethoxy)thiophene-2-carboxylic acid (120 mg, 80.87% yield) as a white solid.

### Step 3 Synthesis of tert-butyl (S)-(1-(5-bromo-3-(trifluoromethoxy)thiophene-2-carbonyl)pyrrolidin-3-yl)carbamate

To a solution of 5-bromo-3-(trifluoromethoxy)thiophene-2-carboxylic acid (120 mg, 0.4123 mmol), tert-butyl (S)-pyrrolidin-3-ylcarbamate (77 mg, 0.4123 mmol) and NMI (119 mg, 1.443 mmol) in MeCN (5 mL) was added TCFH (174 mg, 0.6184 mmol), the mixture was stirred at 25° C. for 2 hours. After completion, the solvent was removed under reduced pressure and the residue was purified by flash column chromatography using petroleum ether/EtOAc=1/1 as eluent to afford tert-butyl (S)-(1-(5-bromo-3-(trifluoromethoxy)thiophene-2-carbonyl)pyrrolidin-3-yl)carbamate (90 mg, 45.16% yield) as a white solid.

LCMS (ESI) calculated for C₁₅H₁₉BrF₃N2O₄S⁺ [M+H]⁺ m/z 459.02, found 458.9.

### Step 4 Synthesis of tert-butyl (S)-(1-(5-(3-chloro-4-(1-(tetrahydro-2H-pyran-4-yl)piperidin-4-yl)phenyl)-3-(trifluoromethoxy)thiophene-2-carbonyl)pyrrolidin-3-yl)carbamate

To a solution of 4-(2-chloro-4-(4,4,5,5-tetramethyl-1,3,2-dioxaborolan-2-yl)phenyl)-1-(tetrahydro-2H-pyran-4-yl)piperidine (100 mg, 0.2464 mmol), tert-butyl (S)-(1-(5-bromo-3-(trifluoromethoxy)thiophene-2-carbonyl)pyrrolidin-3-yl)carbamate (113 mg, 0.2464 mmol) and K₃PO₄ (157 mg, 0.7392 mmol) in 1,4-dioxane/H₂O (10 mL, v/v=8/1) was added Pd(dppf)Cl₂DCM (20 mg, 0.02464 mmol), the mixture was stirred at 95° C. for 16 hours. After completion, the solvent was removed under reduced pressure and the residue was purified by column chromatography using dichloromethane/MeOH=10/1 as eluent to afford tert-butyl (S)-(1-(5-(3-chloro-4-(1-(tetrahydro-2H-pyran-4-yl)piperidin-4-yl)phenyl)-3-(trifluoromethoxy)thiophene-2-carbonyl)pyrrolidin-3-yl)carbamate (130 mg, 64.12% yield) as a dark oil.

LCMS (ESI) calculated for C₃₁H₄₀ClF₃N₃O₅S [M+H]⁺ m/z 658.8, found 658.8.

### Step 5 Synthesis of (S)-(3-aminopyrrolidin-1-yl)(5-(3-chloro-4-(1-(tetrahydro-2H-pyran-4-yl)piperidin-4-yl)phenyl)-3-(trifluoromethoxy)thiophen-2-yl)methanone (Compound 252)

To a solution of tert-butyl (S)-(1-(5-(3-chloro-4-(1-(tetrahydro-2H-pyran-4-yl)piperidin-4-yl)phenyl)-3-(trifluoromethoxy)thiophene-2-carbonyl)pyrrolidin-3-yl)carbamate (30 mg, 0.0456 mmol) in EtOAc (5 mL) was added HCl (3 mL, 2N in EtOAc), the mixture was stirred at 25° C. for 16 hours. After completion, the mixture was concentrated under reduced pressure to afford (S)-(3-aminopyrrolidin-1-yl)(5-(3-chloro-4-(1-(tetrahydro-2H-pyran-4-yl)piperidin-4-yl)phenyl)-3-(trifluoromethoxy)thiophen-2-yl)methanone (14.8 mg, 56.36% yield) as a yellow solid.

LCMS (ESI) calculated for C₂₆H₃₂ClF₃N3O₃S⁺ [M+H]⁺ m/z 558.2, found 558.2.

¹H NMR (400 MHz, MeOD) δ 7.79 (s, 1H), 7.55 (s, 1H), 7.46 (d, J=7.8 Hz, 1H), 7.39 (d, J=7.8 Hz, 1H), 3.98 (dd, J=19.4, 15.3 Hz, 4H), 3.65 (d, J=12.8 Hz, 5H), 3.38 (t, J=11.6 Hz, 4H), 3.17 (s, 2H), 2.38 (s, 1H), 2.16-1.92 (m, 7H), 1.77 (dd, J=15.8, 7.9 Hz, 2H), 1.19 (s, 2H).

### Example 89: Synthesis of (S)-(3-aminopyrrolidin-1-yl)(3-bromo-5-(4-(1-(tetrahydro-2H-pyran-4-yl)piperidin-4-yl)phenyl)thiophen-2-yl)methanone (Compound 255)

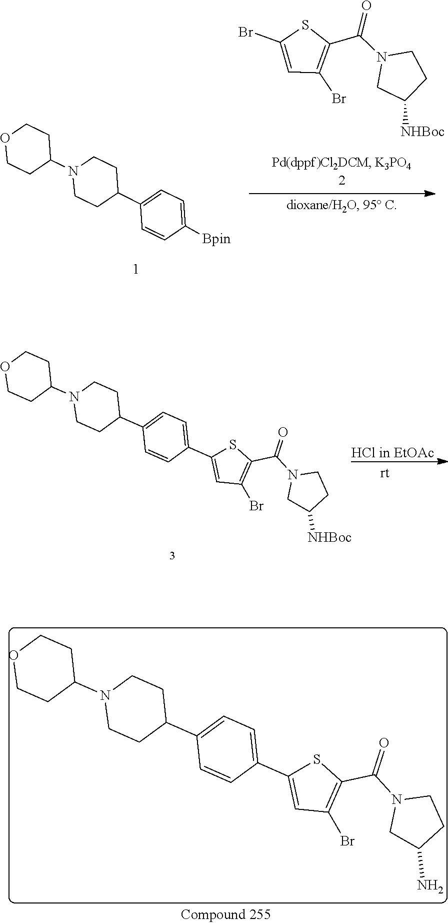

### Step 1 Synthesis of tert-butyl (S)-(1-(3-bromo-5-(4-(1-(tetrahydro-2H-pyran-4-yl)piperidin-4-yl)phenyl)thiophene-2-carbonyl)pyrrolidin-3-yl)carbamate

To a solution of 1-(tetrahydro-2H-pyran-4-yl)-4-(4-(4,4,5,5-tetramethyl-1,3,2-dioxaborolan-2-yl)phenyl)piperidine (1.2 g, 3.2 mmol), tert-butyl (S)-(1-(3,5-dibromothiophene-2-carbonyl)pyrrolidin-3-yl)carbamate (1.6 g, 3.5 mmol) and K₃PO₄ (2 g, 9.6 mmol) in 1,4-dioxane/H₂O (20 mL, v/v=8/1) was added Pd(dppf)Cl₂DCM (0.3 g, 0.32 mol), the mixture was stirred at 95° C. under N₂ for 16 h. After completion, the mixture was concentrated and the residue was purified by flash column chromatography (PE/EA=1/1) to afford tert-butyl (S)-(1-(3-bromo-5-(4-(1-(tetrahydro-2H-pyran-4-yl)piperidin-4-yl)phenyl)thiophene-2-carbonyl)pyrrolidin-3-yl)carbamate (690 mg, 34.38% yield) as red solid.

LCMS (ESI) calculated for C₃₀H₄₀BrN3O₄S⁺ [M+H]⁺ m/z 620.19, found 620.05.

### Step 2 Synthesis of (S)-(3-aminopyrrolidin-1-yl)(3-bromo-5-(4-(1-(tetrahydro-2H-pyran-4-yl)piperidin-4-yl)phenyl)thiophen-2-yl)methanone (Compound 255)

To a solution of tert-butyl (S)-(1-(3-bromo-5-(4-(1-(tetrahydro-2H-pyran-4-yl)piperidin-4-yl)phenyl)thiophene-2-carbonyl)pyrrolidin-3-yl)carbamate (150 mg, 0.2425 mmol) in EtOAc (5 mL) was added HCl (5 mL, 2N in EtOAc), the mixture was stirred at 25° C. for 16 hours. After completion, the solvent was removed under reduced pressure and purified by prep-HPLC to afford (S)-(3-aminopyrrolidin-1-yl)(3-bromo-5-(4-(1-(tetrahydro-2H-pyran-4-yl)piperidin-4-yl)phenyl)thiophen-2-yl)methanone (42 mg, 34.19% yield) as a yellow solid.

LCMS (ESI) calculated for C₂₅H₃₂BrN₃O₂S⁺ [M+H]⁺ m/z 520.14, found 520.00.

¹H NMR (400 MHz, MeOD) δ 7.64 (d, J=8.2 Hz, 2H), 7.39 (s, 1H), 7.36 (d, J=8.2 Hz, 2H), 4.09 (dd, J=11.5, 4.2 Hz, 2H), 4.04-3.96 (m, 2H), 3.79-3.67 (m, 5H), 3.47 (t, J=11.4 Hz, 3H), 3.18 (t, J=11.7 Hz, 2H), 2.96 (t, J=12.3 Hz, 1H), 2.46 (dd, J=13.6, 6.4 Hz, 1H), 2.18 (d, J=13.7 Hz, 3H), 2.05 (dd, J=24.9, 12.2 Hz, 4H), 1.88-1.78 (m, 2H).

### Example 90: In Vitro Efficacy Assay of Exemplary Compounds—TDP-43

Exemplary compounds disclosed herein were evaluated for efficacy in inhibiting TDP-43 inclusions cellular imaging-based assays.

The cellular model was in human neuroblastoma SH SYSY cells (ATCC, cat #: CRL-2266). The parent cell line was first engineered to stably express a tetracycline repressor protein (designated as TREx-SY5Y cells, customer cell line development by ThermoFisher). Wild-type human TDP-43 with a C-terminus eGFP tag was synthesized and cloned into pcDNA5/TO expression vector (ThermoFisher). The plasmid was transfected into TREx-SY5Y cells using Lipofectamine 2000 and hygromycin resistant colonies were selected. Expression of TDP-43WT::eGFP was examined under fluorescent microscopy and Western blot upon tetracycline induction. The clone that was used for TDP-43 aggregation assay is referred to as SY5Y TDP-43WT cells hereafter.

Compound inhibition of TDP-43 aggregation was tested in an 8-point dose curve in 96-well format. Briefly, TDP-43WT::eGFP expression was induced with 1 M tetracycline for 24 hrs (SY5Y TDP-43WT cells). The cells were then pre-treated for 1 hour with exemplary compounds before adding sodium arsenite to a final concentration of 15 M and incubated for another 23 hrs. At the end of the treatment the cell monolayers were washed in PBS and fixed in 4% paraformaldehyde (PFA), diluted from 16% stock (Electron Microscopy Sciences cat: #15710-S). The inhibitory effect on TDP-43 aggregation was measured using CellInsight CX7 high content imager (ThermoFisher). The percentage of cells with TDP-43 aggregates was calculated based on the total number of cells identified by DAPI staining. An 8-point dose response curve was generated, and the IC₅₀ for each compound tested was determined (Table 3).

In Table 3 below, A indicates an IC₅₀ (nM)<500 nM, B indicates an IC₅₀ (nM) 500 nM to <1000 nM, C indicates an IC₅₀ (nM) 1000 nM to <2000 nM, D indicates an IC₅₀ (nM) 2000 nM to <6000 nM, and E indicates an IC₅₀ (nM) 6000 nM to ≤10000 nM.

TABLE 3
Inhibition of TDP-43 Aggregation

| Compound No | IC₅₀ |
|---|---|
| 136 | A |
| 139 | A |
| 141 | A |
| 142 | A |
| 154 | B |
| 155 | A |
| 156 | A |
| 157 | E |
| 171 | B |
| 172 | A |
| 173 | A |
| 174 | C |
| 175 | A |
| 176 | A |
| 177 | B |
| 178 | A |
| 179 | A |
| 180 | A |
| 181 | A |
| 182 | A |
| 183 | A |
| 184 | D |
| 185 | A |
| 186 | D |
| 187 | B |
| 188 | A |
| 189 | A |
| 190 | A |
| 191 | A |
| 192 | D |
| 193 | B |
| 194 | B |
| 195 | C |
| 196 | E |
| 197 | C |
| 198 | A |
| 199 | A |
| 200 | A |
| 201 | A |
| 202 | C |
| 203 | D |
| 204 | E |
| 205 | B |
| 218 | A |
| 219 | A |
| 220 | E |
| 223 | C |
| 224 | E |
| 225 | A |
| 227 | D |
| 228 | B |
| 229 | B |
| 230 | D |
| 231 | D |
| 232 | B |
| 233 | E |
| 234 | D |
| 235 | A |
| 236 | A |
| 237 | B |
| 238 | E |
| 239 | B |
| 240 | C |
| 241 | E |
| 242 | A |
| 243 | A |
| 244 | D |
| 245 | E |
| 246 | E |
| 247 | E |
| 248 | B |
| 249 | A |
| 250 | A |
| 251 | A |
| 252 | E |
| 253 | A |
| 254 | A |
| 255 | A |
| 256 | A |
| 257 | A |
| 258 | A |
| 259 | D |
| 260 | C |
| 261 | C |
| 262 | A |
| 263 | A |
| 264 | A |
| 265 | A |
| 266 | A |
| 267 | A |
| 268 | A |
| 269 | A |
| 270 | A |
| 271 | A |
| 272 | C |
| 273 | A |
| 274 | A |
| 275 | A |
| 276 | A |
| 277 | A |
| 278 | A |
| 279 | A |
| 280 | A |
| 281 | A |
| 282 | A |
| 283 | A |
| 284 | A |
| 285 | C |
| 286 | A |
| 287 | A |
| 288 | A |
| 289 | A |
| 290 | A |
| 291 | A |
| 292 | B |
| 293 | B |
| 294 | A |
| 295 | A |
| 296 | A |

### Example 91: In Vitro Efficacy Assay of Exemplary Compounds—SY5Y-tT1 Tau Arsenite/Thapsigargin Assay

### Day 1—Harvesting and Plating Cells:

Before harvesting cells—checked to see if cells are ~80% confluency in their flask
   1. Aspirated growth media from flask.
   2. Added 4 mL of (0.25%) trypsin-EDTA (REF: 25200-056)
   3. Placed in 37° C. incubator for 5 minutes.
   4. Removed from incubator and added 11 mL growth media, washing any remaining cells adhered to the bottom of the flask.
   5. Transferred cell containing media to a 50 mL conical tube and spun for 5 minutes at 800 rpm.
   6. Aspirated supernatant and added 5 mL of growth media to cell pellet, triturating to make a homogenous cell solution.
   7. After counting cells, followed one of these plating paradigms:
      (A) If treating with arsenite, plated cells at 2,0000 cells/well with tetracycline solution (5 mg/mL) added to cell solution at 1:5000
      (B) If treating with thapsigargin, plated cells at 4,000 cells/well with tetracycline solution (5 mg/mL) added to cell solution at 1:5000
   8. Incubated at 37° C. for 48 hours.

### Day 3:

### Part 1—Compound Treatment:

Solubilized all compounds to 10 mM prior to making dilution plates
   1. To a 96-well U-bottom plate (Falcon Non-Tissue Culture Treated Plates REF: 351177) added 120 μL growth media to all 96 wells, with an additional 58.2 μL to row A.
   2. Added 1.8 μL of 10 mM compound stock to wells A1-11, adding 1.8 μL of 10 mM PERKi stock (Selleck Chemistry GSK2606414 Cat No. S7307) as the control.
   3. Serially diluted 60 μL from row A down through each row, finishing with 180 μL total volume in row H.
   4. Transferred 12.5 μL of each dilution plate well to the correlating wells in cell plate, being careful not to touch cells adhered to bottom of the plate.
   5. Incubated for 2 hours.

### Part 2—Stressor Treatment:

### Arsenite Treatment:

1. Added 720 μL of 0.05 M sodium arsenite solution (CAS #: 7784-46-5) to 10 mL of growth media for a final concentration of 3.6 mM arsenite solution, mix well.
2. Added 12.5 μL of arsenite solution to all wells of compounds treated plate intended for arsenite treatment.
3. Incubated at 37° C. for 90 minutes.

### Thapsigargin Treatment:

1. Added 100 μL of 5 mg/mL thapsigargin (Selleck Chem Cat No. S7895) stock concentration to 10 mL, mix well.
2. Added 12.5 μL of thapsigargin solution to all compound treated plates intended for thapsigargin treatment.
3. Incubated for 90 minutes.

### Part 3—Fixation and DAPI Staining

Before beginning this part of the protocol:
   Made 1×PBS by diluting 10× stock (10×, Phosphate buffered saline, Fisher #BP399—
      4) in sterile deionized water.
      Diluted paraformaldehyde (16% Paraformaldehyde solution, Electron Microscopy Sciences #15710-S) to 4% in 1×PBS.
      Diluted Hoechst stain (Life Technologies #H3570) 1:10,000 in 1×PBS (1 μL Hoechst per 10 mL 1×PBS).
Both solutions were protected from light.
1) After compound treatment, aspirated media from cell plates and gently added in 50 μL of 4% paraformaldehyde for 10 min protected from light at room temperature.
2) Aspirated paraformaldehyde solution and gently added 50 μL of 1:10,000 Hoechst solution to each well and incubated for 10 min protected from light at room temperature.
3) Aspirated Hoechst solution and gently added 100 μL 1×PBS. Cover the plate with aluminum film (VWR #60941-126).
4) Stored at 4° C. or used immediately for imaging on ThermoFisher CX7 imager.

Exemplary compounds of the disclosed herein were evaluated for efficacy for inhibitor activity of tau stress granules in the arsenite assay and quantified as drug concentration providing a 50% reduction (IC₅₀) of granules per cell is shown in Table 4.

In Table 4 below, A indicates an IC₅₀ (μM)<0.5 μM, B indicates an IC₅₀ (μM) 0.5 μM to <1 μM, C indicates an IC₅₀ (μM) 1 μM to <2 μM, and D indicates an IC₅₀ (μM) 2 μM to <6 μM.

TABLE 4
Inhibitor Activity of Tau Stress Granules

| Compound No | IC₅₀ |
|---|---|
| 103 | D |
| 104 | C |
| 108 | B |
| 109 | A |
| 110 | C |
| 111 | D |
| 112 | D |
| 117 | C |
| 118 | C |
| 119 | B |
| 120 | C |
| 124 | A |
| 128 | C |
| 129 | D |
| 134 | D |
| 135 | D |
| 136 | A |
| 139 | B |
| 140 | B |
| 141 | B |
| 142 | B |
| 144 | D |
| 147 | A |
| 148 | C |
| 149 | A |
| 151 | D |
| 152 | C |
| 153 | C |
| 155 | B |
| 156 | A |
| 158 | C |
| 159 | B |
| 162 | C |
| 164 | A |
| 165 | B |
| 166 | A |
| 167 | C |
| 176 | B |
| 178 | A |
| 179 | B |
| 180 | A |
| 181 | B |
| 182 | A |
| 183 | A |
| 198 | D |
| 199 | D |
| 200 | D |
| 201 | A |
| 217 | C |

### Example 92. Efficacy of Compound 183 in a Stem Cell-Derived Motor Neuron Model of TDP-43 Proteinopathy

Cell culture and differentiation of iPS Cells into motor neurons. Pluripotent stem cells were grown with mTeSR Plus medium on tissue culture dishes coated with Matrigel, and maintained in 5% CO₂ incubators at 37° C. Stem cells were passaged as small aggregates of cells after 1 mM EDTA treatment. 10 M ROCK inhibitor was added to the cultures for 16-24 h after dissociation to prevent cell death. The iP11NA iPS cell line was acquired from ALSTEM. This cell line was generated from pre-made footprint-free human iPS cells with DOX inducible Ngn2 that has been stably integrated into the human AAVS1 “Safe Harbor” site, and provides a convenient means to differentiate human iPS cells into neurons rapidly and efficiently. Motor neuron differentiation was achieved using a modified 5-day strategy.

This approach relies on neural induction through small molecule inhibition of SMAD signaling, accelerated neural differentiation through forced Ngn2 expression, and motor neuron patterning through the activation of retinoic acid and Sonic Hedgehog signaling pathways. In brief, pluripotent stem cells were dissociated to single cells using accutase and plated at a density of 80,000 cells per cm² on Matrigel-coated culture plates with mTeSR Plus medium supplemented with ROCK inhibitor (10 μM Y-27632, ATCC). When cells reached 50% confluency, medium was changed to differentiation medium BrainPhys medium supplemented with N2-A, SM1, 10 M Ascorbic Acid and 10 M db-cAMP. This time point was defined as day 0 (d₀) of motor neuron differentiation. Treatment with small molecules was carried out as follows: 2 μg/mL Doxycyline, 10 μM SB431542, 100 nM LDN-193189, 1 M retinoic acid and 1 μM Smoothend agonist and 10 uM Edu. The full list of materials and their descriptions is included below in Table 5.

TABLE 5
List of Materials Used in Assay

| Reagent | Description | Brand |
|---|---|---|
| Hoechst 33342 | immunofluorescence | Invitrogen |
| Anti-TDP-43 | antibody | Proteintech |
| Human Nuclear | antibody | NeoBiotechnologies |
| Antigen Monoclonal | | |
| Antibody (235-1) | | |
| Anti-Beta-Tubulin 3 | immunoblot | Aves Labs |
| Antibody | | |
| MG-132 | proteasome inhibitor | Sigma-Aldrich |
| Subcellular Protein | immunoblot | Life Technologies |
| Fractionation Kit | | Corp |
| TaqMan Fast | RT-PCR | Thermo Fisher |
| Advanced Cells-to- | | |
| CT Kit | | |
| 4X Bolt LDS | Immunoblot | Invitrogen |
| Sample Buffer | | |
| 10X Bolt Sample | Immunoblot | Invitrogen |
| Reducing Agent | | |
| Bolt Bis-Tris Plus | Immunoblot | Invitrogen |
| Mini Protein Gels | | |
| 20X Bolt MES SDS | Immunoblot | Invitrogen |
| Running Buffer | | |
| Precision Plus | Immunoblot | Biorad |
| Protein | | |
| Kaleidoscope | | |
| Ladder | | |
| iBlot 2 Transfer | Immunoblot | Invitrogen |
| Stacks, PVDF, mini | | |
| Intercept (TBS) | Immunoblot | LI-COR |
| Blocking Buffer | | Biotechnology |
| Tris Buffered Saline | Immunoblot | Cell Signaling |
| with Tween | | Technology |
| Intercept T20 | Immunoblot | LI-COR |
| (TBS) Protein-Free | | Biotechnology |
| Antibody Diluent | | |
| Sodium dodecyl | Immunoblot | Boston Bioproducts |
| sulfate 20% | | |
| IRDye 680RD | | LI-COR |
| Donkey anti-Mouse | | Biotechnology |
| IgG Secondary | | |
| Antibody | | |
| IRDye 800CW | Immunoblot | LI-COR |
| Donkey anti-Rabbit | | Biotechnology |
| IgG Secondary | | |
| Antibody | | |
| mTesR plus | IPSC maintenance | Stemcell Tech |
| ReleSR | IPSC maintenance | Stemcell Tech |
| Gentle cell | IPSC maintenance | Stemcell Tech |
| dissociation | | |
| reagent | | |
| BrainPhys Neuronal | MN differentiation | Stemcell Tech |
| Medium N2-A & | | |
| SM1 Kit | | |
| Accutase, 100 mL | MN differentiation | Stemcell Tech |
| L-Ascorbic acid | MN differentiation | SIGMA |
| Dibutyryl-cAMP | MN differentiation | Stemcell Tech |
| Doxycycline | MN differentiation | Stemcell Tech |
| (hyclate) | | |
| SB431542 | MN differentiation | Selleckchem |
| LDN193189 | MN differentiation | Advanced |
| | | Chemblocks Inc |
| Retinoic acid | MN differentiation | SIGMA |
| SAG | MN differentiation | Stemcell Tech |
| EdU (5-ethynyl-2′- | MN differentiation | Invitrogen |
| deoxyuridine) | | |
| Human Recom | MN differentiation | Stemcell Tech |
| BDNF | | |
| Human Recom | MN differentiation | Stemcell Tech |
| GDNF | | |
| Human Recom | MN differentiation | Stemcell Tech |
| CNTF | | |
| ROCK Inhibitor | IPSC maintenance + | ATCC |
| Y27632 | MN differentiation | |
| Poly-L-ornithine | Coating | SIGMA |
| solution | | |
| Laminin | Coating | Life tech |
| Geltrex LDEV FREE | Coating | Life tech |
| hESC Qual | | |

### TDP-43 Localization Analysis

For immunofluorescence, cells were fixed with 4% PFA for 15 min, and permeabilized with 0.1% Triton-X-100 in ×1 PBS for 15 min. Cells were then incubated overnight at 4° C. with primary antibody (diluted in blocking solution—0.5% BSA). At least 1 wash was carried out, before incubating the cells with secondary antibodies for 1 h at room temperature (diluted in blocking solution). Nuclei were stained with Hoechst. The following antibodies were used in this study: TUJI (1:1500, R&D Systems MAB NL493), Human Nuclear Antigen (1:200, Thermo MSM3-99-P0), TDP-43 (1:500 ProteinTech Group). Secondary antibodies used (488, 555, 594 and 647) were AlexaFluor. For analysis of TDP-43 nuclear localization analysis, human motor neurons images were acquired using a CellInsight™ CX7 High Content Analysis Platform. Microtubule staining was used to determine the cell body as the region of interest and then Hoechst staining to identify the nuclear region of interest. The mean TDP-43 intensity in these two regions were used to calculate the nuclear to cytoplasmic ratio for TDP-43 immunofluorescence.

### Cell Fractionation and Immunoblot Assays

For analysis of TDP-43 cellular distribution, d14 motor neurons were lysed with Thermo Subcellular Protein Fractionation Kit for cultured cells and processed according to the manufacturer's instructions. 3 million neurons per well of 6-well culture were routinely used for processing. After one wash with ice-cold PBS, neurons were lysed in 400 μL of cytoplasmic extraction buffer. For immunoblot assays 2:1:1 μL of cytoplasmic extraction buffer:nuclear extraction buffer:pellet extraction buffer were separated by Bolt™ Bis-Tris Plus Mini Protein Gels, 4-12%, 1.0 mm, WedgeWell format, transferred to polyvinylidenedifluoride membranes and probed with antibodies against TDP-43 (1/1,000, ProteinTech Group). Fluorescent secondary antibodies were used for band detection and the Kaleidoscope precision plus protein ladder for size estimation. iBright FL1500 imaging system and software were used to quantitate protein band signal.

### RNA Preparation and Quantitative RT-PCR

Total RNA was isolated from d14 motor neurons for qRT-PCR assays using TaqMan™ Fast Advanced Cells-to-CT™ Kit according to manufacturer's instructions. 7500 neurons per well of 96-well culture were routinely used for processing. qRT-PCR was then performed using multiplex assays from IDT using PrimeTime™ Gene Expression Master Mix (IDT) on a QuantStudio6Pro system. Quantitative levels for all genes assayed were normalized using GAPDH expression. Normalized expression was displayed relative to the relevant control sample using the comparative DDCt method. Primers and probes used in this study are in Table 5.

### Results

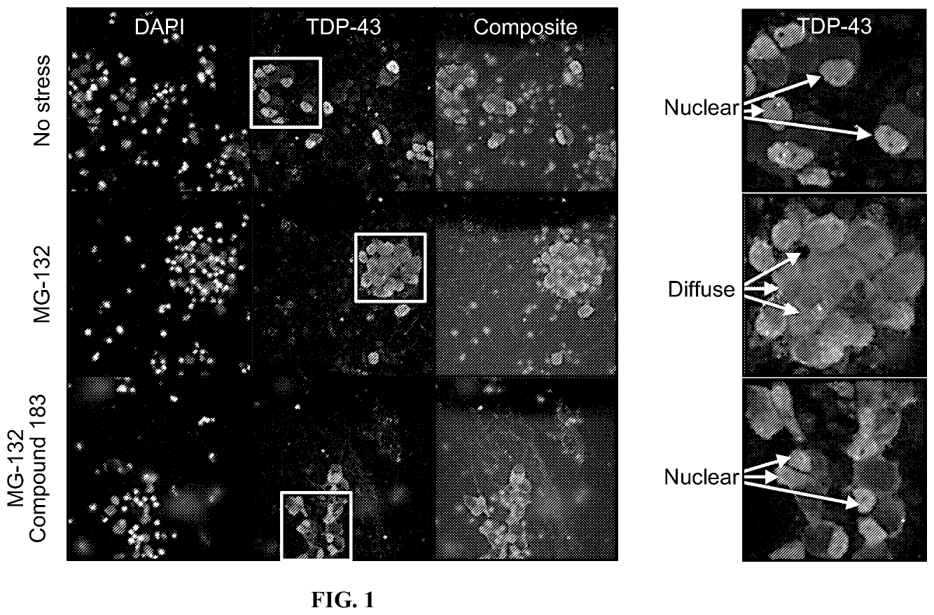

Compound 183 prevents nuclear loss of TDP-43. A paucity nuclear TDP-43 staining is a widespread pathological hallmarks of ALS8. Inhibition of the proteasome has been reported to induce nuclear loss of TDP-436,99. Thus, we explored whether MG-132-mediated proteasome inhibition could influence TDP-43 localization in human motor neurons and serve as a potential model of acute TDP-43 dysfunction. Indeed, inhibition of the proteasome using 1 μM MG-132 for 20 hours induced a loss of nuclear TDP-43 staining as measured by immunofluorescence (see FIG. 1). Strikingly, if the neurons were pretreated with 500 nM Compound 183 for 1 hour and then during the duration of proteasome inhibition, TDP-43 staining in the nucleus was observed that was much more similar to that of unchallenged neurons (see FIG. 1). These data indicate that Compound 183 can prevent nuclear loss of TDP-43 in human motor neurons.

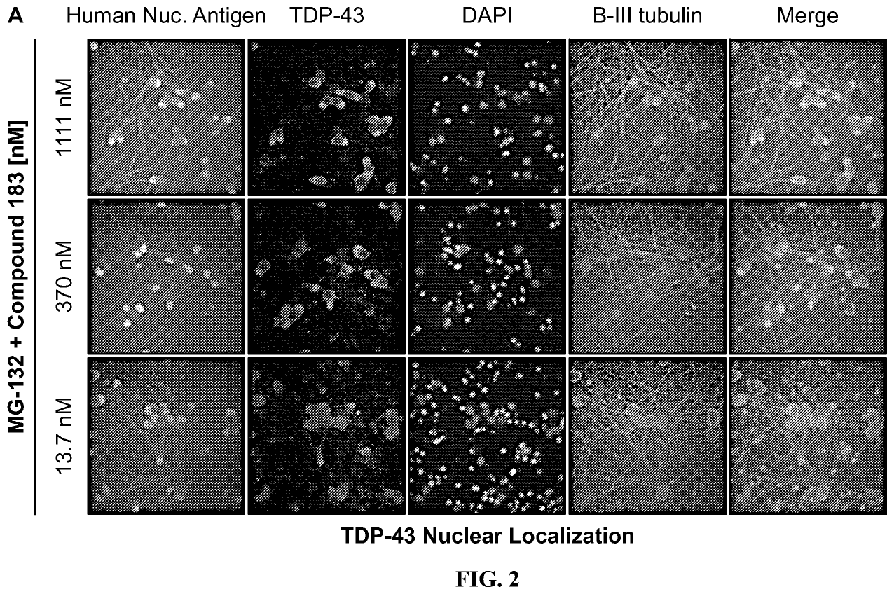

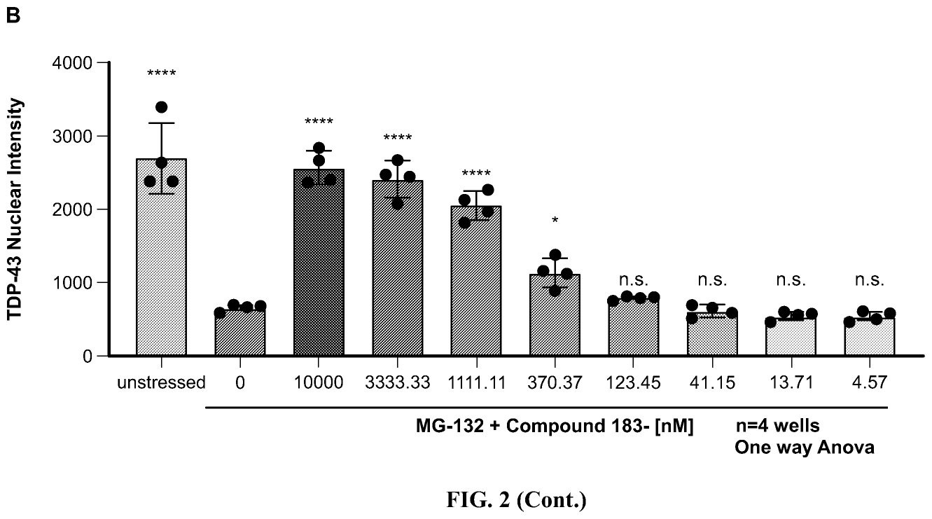

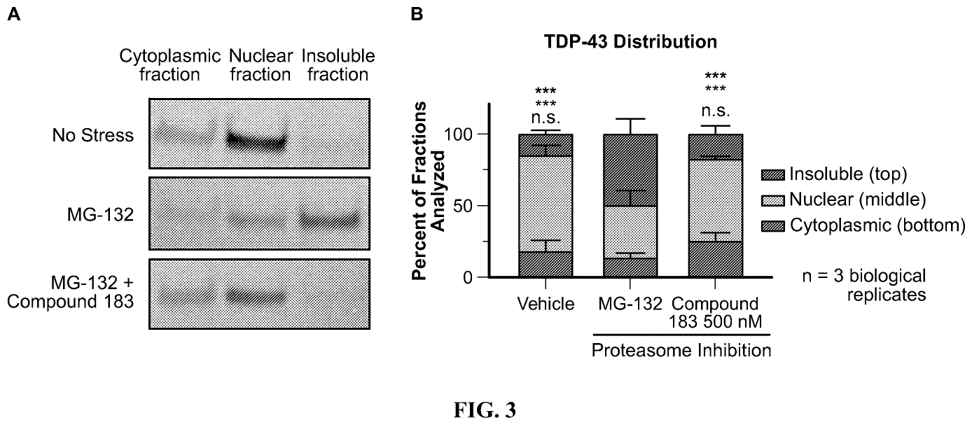

A dose-responsive effect was further tested for Compound 183 using quantitative imaging analysis on the CellInsight™ CX7 High Content Analysis Platform. By comparing the intensity of TDP-43 staining in the nucleus and cytoplasm of human motor neurons, we determined Compound 183 could maintain nuclear TDP-43 from 370 nM-10 μM in the proteasome challenged motor neurons (see FIG. 2). These data indicate that Compound 183 can reproducibly prevent nuclear loss of TDP-43 in human motor neurons in a dose-dependent manner.

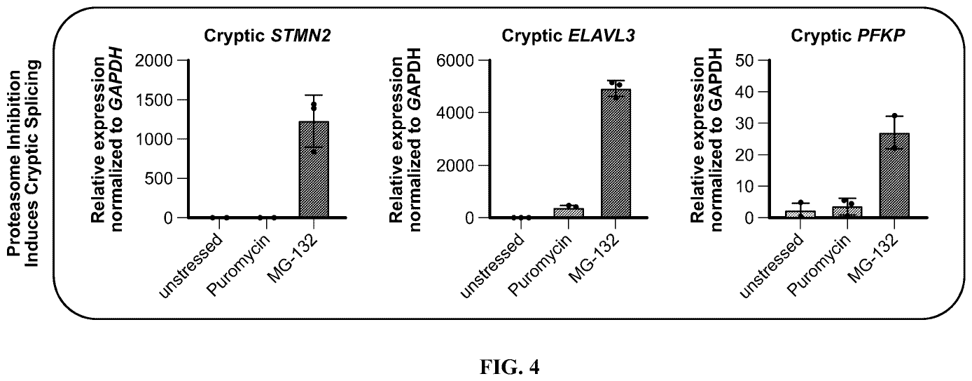

Compound 183 prevents accumulation of insoluble TDP-43. In ALS patients, TDP-43 mislocalization is associated with accumulation of insoluble TDP-43 protein. After proteasome inhibition in human motor neurons, we examined TDP-43 cellular distribution by fractionating cell homogenates to different cellular compartments including the cytoplasmic, nuclear, and insoluble compartments. In lysates from neurons treated with MG-132, we observed a significant change in TDP-43 distribution from the soluble, nuclear compartment to the insoluble fraction. These data are consistent with published findings that described a shift of TDP-43 from a soluble to an insoluble compartment after proteasome inhibition. After confirming that blocking proteasome activity induced TDP-43 mislocalization and insolubility, we asked if Compound 183 could prevent this conversion of TDP-43 into a pathological state. Impressively, treatment of cells with 500 nM Compound 183 almost completely blocked the accumulation of insoluble TDP-43 in human motor neurons. Additionally, the levels of TDP-43 in the nucleus were maintained (see FIG. 3). Collectively, the data demonstrate that Compound 183 can maintain TDP-43 in the nucleus and prevent its conversion into a potentially pathogenic insoluble aggregate.

Compound 183 broadly restores splicing function of TDP-43 that is lost in ALS. Nuclear TDP-43 loss-of-function has emerged over recent years as a strong potential contributor to ALS pathogenesis with clear, molecular, postmortem evidence published by many independent groups. When TDP-43 is depleted from the nucleus, pre-mRNAs undergo missplicing events including the erroneous inclusion of intronic sequences into mature mRNA, forming so-called ‘cryptic exons’. Cryptic splicing of TDP-43 regulated transcripts connects TDP-43 pathology with neuropathy, is supported by ALS genetics in genes like UNC13A as a bone fide risk factor, could serve as diagnostic as well as predictive biomarkers, and therapeutic targets for antisense oligonucleotide therapies. Restoring TDP-43 function to suppress cryptic splicing and support normal splicing is, therefore, of incredible therapeutic interest.

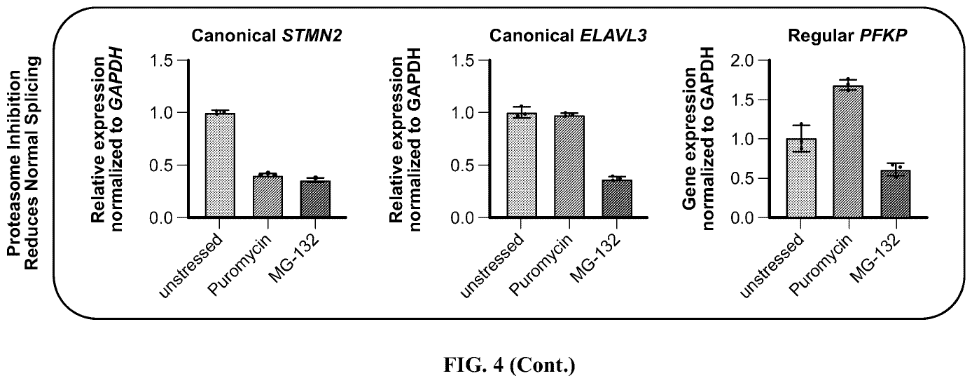

To this end, we first examined whether proteasome inhibition, which triggers TDP-43 nuclear depletion, also leads to splicing defects associated with TDP-43 loss-of function. For comparison, we also treated human motor neurons with puromycin, which recruits TDP-43 to stress granules in the cytoplasm. We chose to analyze several transcripts that have relevant connections to ALS biology including STMN26,12, PFKP19, and ELAVL320,21. We used quantitative reverse transcription-polymerase chain reaction (qRT-PCR) to validate that in MG-132-treated motor neurons these transcripts were altered like in ALS patients. Consistent with the TDP-43 nuclear depletion observed in the cell staining and cellular fractionation experiments (see FIG. 1 and FIG. 2), we observed strong induction of cryptic splicing by qPCR for all three sentinel transcripts with MG-132 (see FIG. 4). Concurrently, we observed decreased levels for normal STMN2 as well as the cryptic exon-disrupted splice junctions in PFKP and ELAVL3. Although puromycin can induce TDP-43 cytoplasmic foci, it failed to induce transcription changes consistent with TDP-43 loss of function and what is observed in ALS patients. These data support the use of MG-132 with stem cell-derived motor neurons as a model of acute TDP-43 dysfunction and for evaluating therapeutics agents for their ability to restore TDP-43 function.

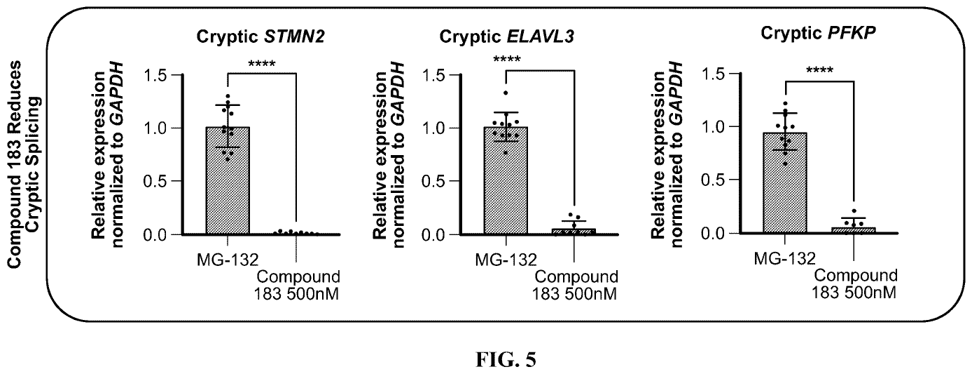

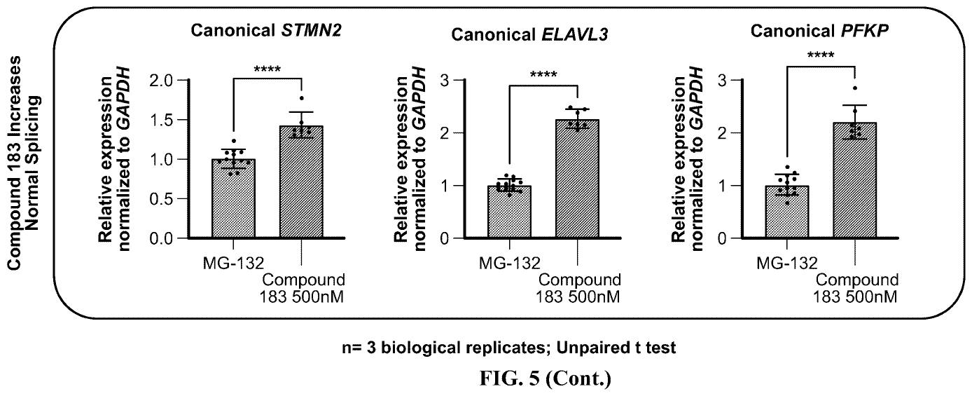

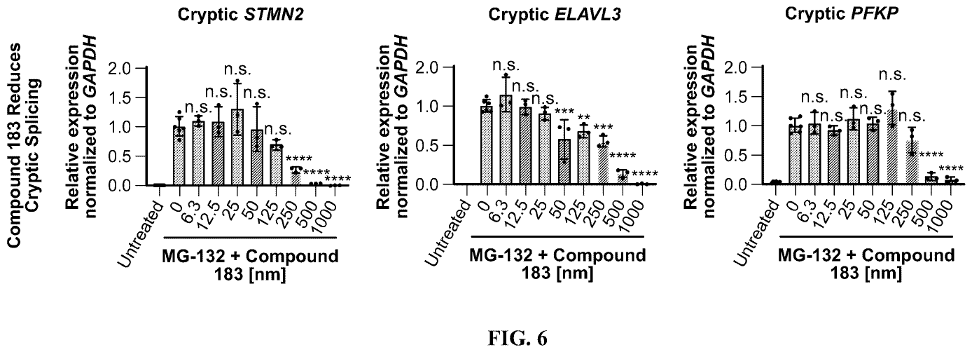

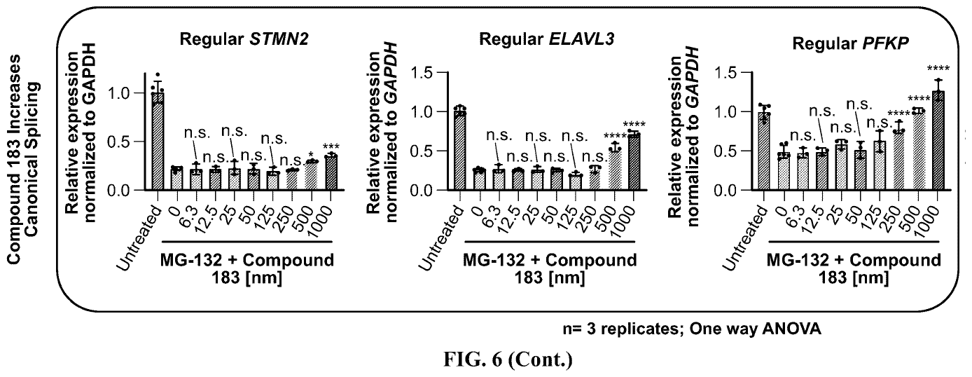

Using this method, we next obtained RNA from independent motor neuron biological replicates after proteasome inhibition and Compound 183 treatment to determined by qRT-PCR the relative abundance of these transcripts. Notably, we confirmed nearly complete inhibition for all three cryptic splicing events after treatment with Compound 183 (see FIG. 5). Importantly, Compound 183 increased splicing of the normal transcripts suggesting the molecule is not reducing global RNA transcription to reduce cryptic splicing but is restoring TDP-43 nuclear function (see FIG. 5). A dose-responsive effect was further tested for Compound 183 using qRT-PCR in this model. By comparing the levels of properly spliced and transcripts containing cryptic exons, we determined Compound 183 could dose dependently maintain TDP-43's splicing function for most transcripts from 250 nM-1000 nM in proteasome challenged motor neurons (see FIG. 6). Although Compound 183 was modest in its ability to restore normal STMN2 and ELAVL3 levels, it fully restored normal splicing of PFKP by 500 nM. Importantly, all transcripts tested displayed strong suppression of cryptic splicing by 500 nM. Collectively, the data support the notion that Compound 183 promotes TDP-43 nuclear function under proteotoxic stress.

### CONCLUSIONS

The acute human motor neurons model of ALS was specifically developed and optimized to assess 3 aspects of ALS pathology: 1) loss of nuclear TDP-43, 2) accumulation of insoluble TDP-43, and 3) splicing changes associated with TDP-43 loss-of-function. Using this model, we have demonstrated that Compound 183 robustly counteracts all three aspects captured by this human disease model. This model and technical report can further be leveraged to address unanswered question regarding Compound 183 target as well as mechanism of action. In summary, we conclude that Compound 183 can restore TDP-43 nuclear localization and normal splicing function, in a dose-dependent manner. These data, together with Compound 183's ability to reduce insoluble TDP-43, point to Compound 183 as a promising therapeutic candidate for ALS and other diseases associated with TDP-43 proteinopathy.

### EQUIVALENTS

It will be recognized that one or more features of any embodiments disclosed herein may be combined or rearranged within the scope of the disclosure to produce further embodiments that are also within the scope of the disclosure.

Those skilled in the art will recognize, or be able to ascertain using no more than routine experimentation, many equivalents to the specific embodiments of the disclosure described herein. Such equivalents are intended to be within the scope of the present disclosure.

Although the compounds and methods disclosed herein been described and illustrated in the foregoing illustrative embodiments, it is understood that the present disclosure has been made only by way of example, and that numerous changes in the details of implementation of the compounds and methods disclosed herein can be made without departing from the spirit and scope of the disclosure, which is limited only by the claims that follow. Features of the disclosed embodiments can be combined or rearranged in various ways within the scope and spirit of the disclosure to produce further embodiments that are also within the scope of the disclosure. Those skilled in the art will recognize, or be able to ascertain, using no more than routine experimentation, numerous equivalents to the specific embodiments described specifically in this disclosure. Such equivalents are intended to be encompassed in the scope of the following claims.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the compounds and methods disclosed and claimed herein.

We claim:

1. A compound of Formula (I): 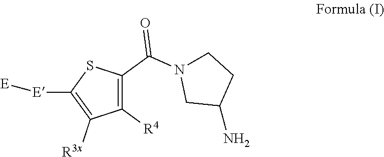
or stereoisomer and/or a pharmaceutically acceptable salt thereof, wherein:
E is C₃-C₇ cycloalkyl, heterocycloalkyl, aryl, heteroaryl, carbocyclyl, or heterocyclyl, and E may be optionally substituted;
E′ is absent, or E′ is C₃-C₇ cycloalkyl, heterocycloalkyl, aryl, heteroaryl, carbocyclyl, or heterocyclyl, and E′ may be optionally substituted; and
R^{3x} and R⁴ are each H or an independently selected optional substituent.

2. The compound of claim 1, wherein E′ is absent.

3. A compound of Formula (II): 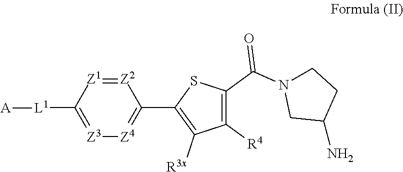
or stereoisomer and/or a pharmaceutically acceptable salt thereof, wherein:
Z¹ is N or CR¹¹;
Z² is N or CR¹²;
Z³ is N or CR¹³;
Z⁴ is N or CR¹⁴;
L¹ is absent, or L² is C₁-C₆ alkylene, C₁-C₆ heteroalkylene, —O—, —S—, or —NR′—, wherein the C₁-C₆ alkylene and C₁-C₆ heteroalkylene are optionally substituted;
A is H, halo, C₁-C₆ alkyl, C₁-C₆ heteroalkyl, C₁-C₆ haloalkyl, C₃-C₇ cycloalkyl, heterocycloalkyl, aryl, heteroaryl, carbocyclyl, or heterocyclyl, and A may be optionally substituted; or A and R¹¹ are taken together with the atoms to which they are attached to form an optionally substituted cycloalkyl, heterocycloalkyl, aryl, heteroaryl, carbocyclic, or heterocyclic ring;
R¹¹, R¹², R¹³, and R¹⁴ are each independently H or an optional substituent;
each R′ is H or C₁-C₆ alkyl; and
each of R^{3x} and R⁴ is independently H or an independently selected optional substituent,
wherein no more than two of Z¹, Z², Z³ and Z⁴ are N.

4. A compound of Formula (II): 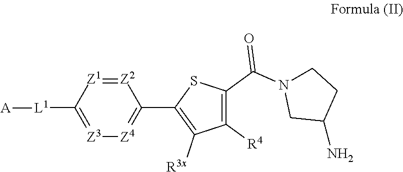
or stereoisomer and/or a pharmaceutically acceptable salt thereof, wherein:
Z¹ is N or CR¹¹;
Z² is N or CR¹²;
Z³ is N or CR¹³;
Z⁴ is N or CR¹⁴;
L¹ is absent, or L¹ is C₁-C₆ alkylene, C₁-C₆ heteroalkylene, —O—, —S—, or —NR′—, wherein the C₁-C₆ alkylene and C₁-C₆ heteroalkylene are optionally substituted by 1-4 independently substituents selected from ═O (oxo), OH, and halogen;
A is H, halo, C₁-C₁₀ alkyl, C₁-C₁₀ heteroalkyl, C₁-C₆ haloalkyl, C₃-C₇ cycloalkyl, 3-7 membered heterocycloalkyl, C₆-C₁₀ aryl, 5-6 membered heteroaryl, C₆-C₁₀ carbocyclyl, or -5-10 membered heterocyclic ring, and A is substituted by one R¹ group and optionally substituted by 1-4 independently selected R² groups; or A and R¹¹ are taken together with the atoms to which they are attached to form a C₃-C₇ cycloalkyl, aryl, 5-6 membered heteroaryl, or 3-7 membered heterocyclic ring, wherein the heteroaryl and heterocyclyl have 1-3 ring heteroatoms selected from N, O, and S, and wherein each of the rings is optionally substituted by one R¹ group and optionally substituted by 1-4 independently selected R² groups;
each of R¹¹, R¹², R¹³, and R¹⁴ is independently H or R³;
R¹ is H, C₁-C₆ alkyl, C₂-C₆ alkenyl, C₂-C₆ alkynyl, C₂-C₉ heteroalkynyl, C₁-C₆ heteroalkyl, C₁-C₆ haloalkyl, —NH₂, or -L²-G, wherein the C₁-C₆ alkyl may be optionally substituted with OH, and wherein the C₁-C₆ heteroalkyl may be optionally substituted with C₂-C₆ heteroalkynyl;
L² is absent, or L² is C₁-C₆ alkylene, C₁-C₆ heteroalkylene, —O—, —S—, or —NR′—, wherein the C₁-C₆ alkylene and C₁-C₆ heteroalkylene are optionally substituted by 1-4 substituents independently selected from ═O (oxo), OH, and halogen;
G is C₃-C₇ cycloalkyl, 3-7 membered heterocycloalkyl, aryl, 5-6 membered heteroaryl, 6-10 membered carbocyclyl, or 5-10 membered heterocyclyl, wherein the 3-7 membered heterocycloalkyl, 5-6 membered heteroaryl, and 5-10 membered heterocyclyl have 1-3 ring heteroatoms selected from N, O, and S, and wherein G may be optionally substituted by 1-4 R^{A} substituents;
each R^{A} is independently selected from the group consisting of C₁-C₆ alkyl, C₁-C₆ heteroalkyl, C₁-C₆ alkoxy, C₁-C₆ haloalkyl, C₁-C₆ haloalkoxy, ═O (oxo), OH, —NMe₂, —NHMe, —NH₂, CN, and halo;
each R² is independently selected from the group consisting of C₁-C₆ alkyl, C₂-C₆ alkenyl, C₂-C₆ alkynyl, C₁-C₆ heteroalkyl, C₁-C₆ alkoxy, C₁-C₆ haloalkyl, C₁-C₆ haloalkoxy, ═O (oxo), —OH, —NMe₂, —NHMe, —NH₂, and halo;
each R³ is independently selected from the group consisting of C₁-C₆ alkyl, C₂-C₆ alkenyl, C₂-C₆ alkynyl, C₁-C₆ alkoxy, C₁-C₆ haloalkyl, C₁-C₆ haloalkoxy, OH, cyano, and halo;
R⁴ is selected from the group consisting of H, C₁-C₆ alkyl, C₂-C₆ alkenyl, C₂-C₆ alkynyl, C₁-C₆ heteroalkyl, C₁-C₆ haloalkyl, C₁-C₆ haloalkoxy, cyano, and halo;
each R′ is H or C₁-C₆ alkyl; and
R^{3x} is selected from the group consisting of H, C₁-C₆ alkyl, C₂-C₆ alkenyl, C₂-C₆ alkynyl, C₁-C₆ alkoxy, C₁-C₆ haloalkyl, cyano, and halo,
wherein no more than two of Z¹, Z², Z³ and Z⁴ are N.

5. The compound of claim 3 or 4, wherein R^{3x} is H or C₁-C₆ alkyl.

6. The compound of claim 3 or 4, wherein R^{3x} is H or -Me.

7. The compound of any one of claims 3 to 6, wherein R⁴ is H, halo, C₁-C₆ haloalkoxy, C₁-C₆ alkyl, C₁-C₆ alkoxy, C₂-C₆ alkenyl, or C₁-C₆ heteroalkyl.

8. The compound of any one of claims 3 to 7, wherein R⁴ is H, —Cl, —Br, —OCF₃, -Me, —OMe, -Et, -nPr, -iPr, 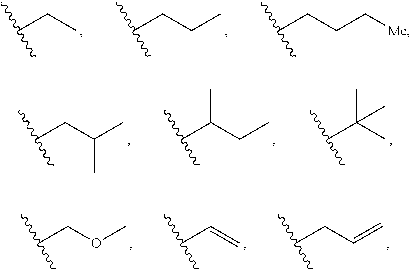
—CH₂OCH₃, —CH═CH₂, or —CH₂CH═CH₂.

9. The compound of any one of claims 3 to 8, wherein Z¹ is CR¹¹, Z² is CR¹², Z³ is CR¹³ and Z⁴ is CR¹⁴.

10. The compound of any one of claims 3 to 8, wherein Z¹ is N, Z² is CR¹², Z³ is CR¹³ and Z⁴ is CR¹⁴.

11. The compound of any one of claims 3 to 8, wherein Z¹ is CR¹¹, Z² is N, Z³ is CR¹³ and Z⁴ is CR¹⁴.

12. The compound of any one of claims 3 to 8, wherein Z¹ is N, Z² is CR¹², Z³ is N, and Z⁴ is CR¹⁴.

13. The compound of any one of claims 3 to 8, wherein Z¹ is N, Z² is N, Z³ is CR¹³, and Z⁴ is CR¹⁴.

14. The compound of any one of claims 3 to 8, wherein Z¹ is CR¹¹, Z² is N, Z³ is CR¹³ and Z⁴ is N.

15. The compound of any one of claims 3 to 14, wherein each of R¹¹, R¹², R¹³, and R¹⁴ is independently H or R³, wherein each R³ is independently selected from the group consisting of C₁-C₆ alkyl, C₁-C₆ alkoxy, C₁-C₆ haloalkyl, C₁-C₆ haloalkoxy, OH, and halo.

16. The compound of any one of claims 3 to 14, wherein each of R¹¹, R¹², R¹³, and R¹⁴ is independently H or R³, wherein each R³ is independently selected from the group consisting of -Me, -Et, -nPr, -iPr, —CF₃, —OMe, —OCF₃, —OH, —F, and —Cl.

17. The compound of any one of claims 3 to 16, wherein L¹ is absent.

18. The compound of any one of claims 3 to 16, wherein L¹ is C₁-C₆ alkylene, C₁-C₆ heteroalkylene, or —O—.

19. The compound of any one of claims 3 to 16, wherein L¹ is —CH₂—, —OCH₂—, —NHCH₂—, —N(CH₃)CH₂—, or —O—.

20. The compound of any one of claims 3 to 19, wherein A is selected from the group consisting of halo, C₁-C₁₀ alkyl, and C₁-C₁₀ heteroalkyl, wherein the C₁-C₁₀ alkyl and C₁-C₁₀ heteroalkyl are optionally substituted by ═O (oxo).

21. The compound of any one of claims 3 to 19, wherein A is selected from the group consisting of —F, —Cl, -Me, -Et, -nPr, -iPr, 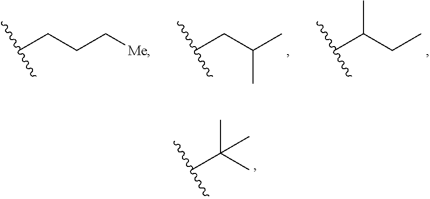
—NHCH₃, —N(CH₃)₂,

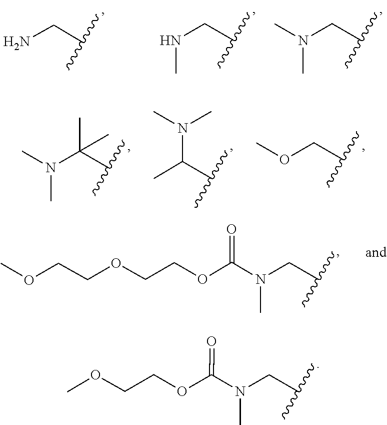

22. The compound of any one of claims 4 to 19, wherein A is selected from the group consisting of C₃-C₇ cycloalkyl, 3-7 membered heterocycloalkyl, C₆-C₁₀ aryl, 5-6 membered heteroaryl, C₆-C₁₀ carbocyclyl, and 5-10 membered heterocyclic ring, and A is substituted by one R¹ group and optionally substituted by 1-4 independently selected R² groups.

23. The compound of any one of claims 4 to 19, wherein A is selected from the group consisting of C₃-C₇ cycloalkyl, 3-7 membered heterocycloalkyl, C₆-C₁₀ aryl, and 5-6 membered heteroaryl, and A is substituted by one R¹ group and optionally substituted by 1-4 independently selected R² groups.

24. The compound of any one of claims 3 to 19, wherein A is selected from the group consisting of

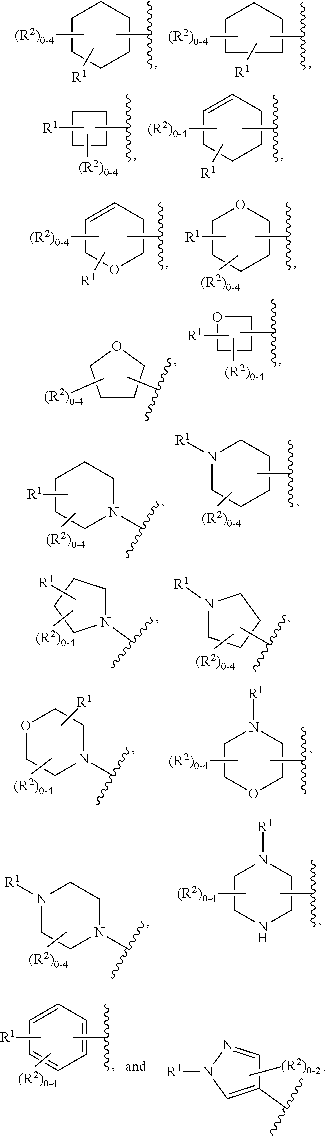

25. The compound of any one of claims 4 to 24, wherein each R² is independently selected from the group consisting of C₁-C₆ alkyl, C₁-C₆ alkoxy, C₁-C₆ haloalkyl, C₁-C₆ haloalkoxy, ═O (oxo), —OH, —NMe₂, —NHMe, —NH₂, and halo.

26. The compound of any one of claims 4 to 24, wherein each R² is independently selected from the group consisting of -Me, -Et, -nPr, -iPr, —CF₃, —OMe, —OCF₃, ═O (oxo), —OH, —NMe₂, —NHMe, —NH₂, F, Cl, and Br.

27. The compound of any one of claims 4 to 24, wherein R¹ is H or -L²-G.

28. The compound of any one of claims 4 to 24, wherein R¹ is -L²-G.

29. The compound of any one of claims 4 to 28, wherein L² is absent.

30. The compound of any one of claims 4 to 28, wherein L² is selected from the group consisting of C₁-C₆ alkylene, C₁-C₆ heteroalkylene, and —O—.

31. The compound of any one of claims 4 to 28, wherein L² is —CH₂—.

32. The compound of any one of claims 4 to 31, wherein G is C₃-C₇ cycloalkyl or 3-7 membered heterocycloalkyl, wherein the 3-7 membered heterocycloalkyl has 1-3 ring heteroatoms selected from N, O, and S, and wherein G may be optionally substituted by 1-4 R^{A} substituents.

33. The compound of any one of claims 4 to 31, wherein G is 3-7 membered heterocycloalkyl, wherein G may be optionally substituted by 1-4 R^{A} substituents.

34. The compound of any one of claims 4 to 31, wherein G is selected from the group consisting of

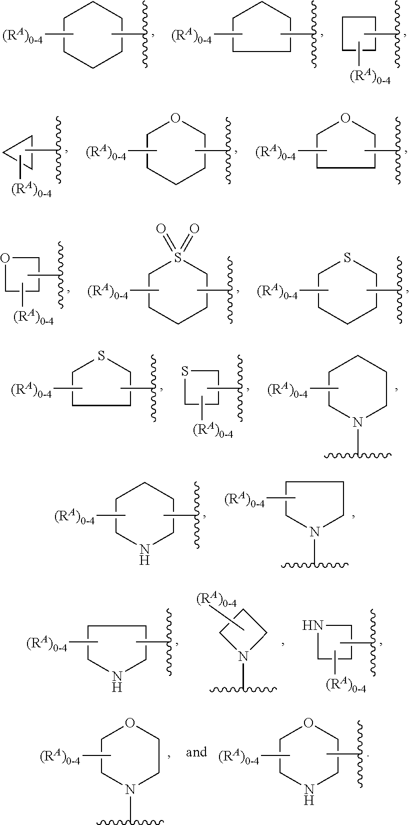

35. The compound of any one of claims 4 to 34, wherein each R^{A} is independently C₁-C₆ alkyl.

36. The compound of any one of claims 4 to 35, wherein G is selected from the group consisting of

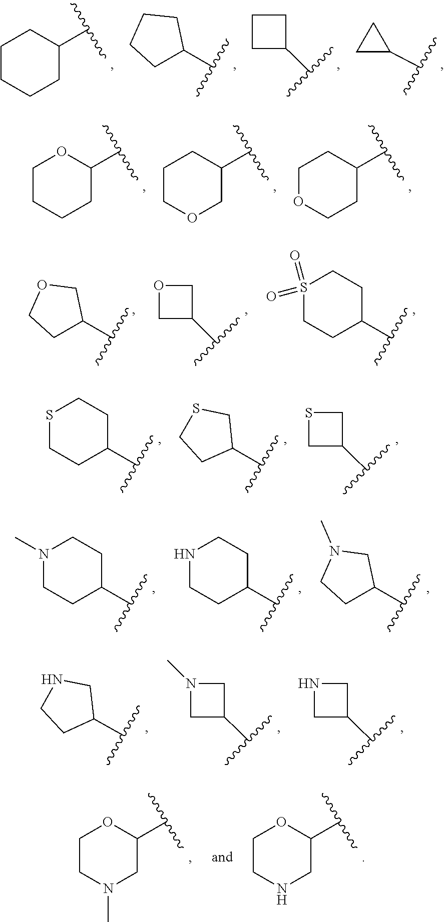

37. The compound of any one of claims 3 to 35, wherein A is selected from the group consisting of

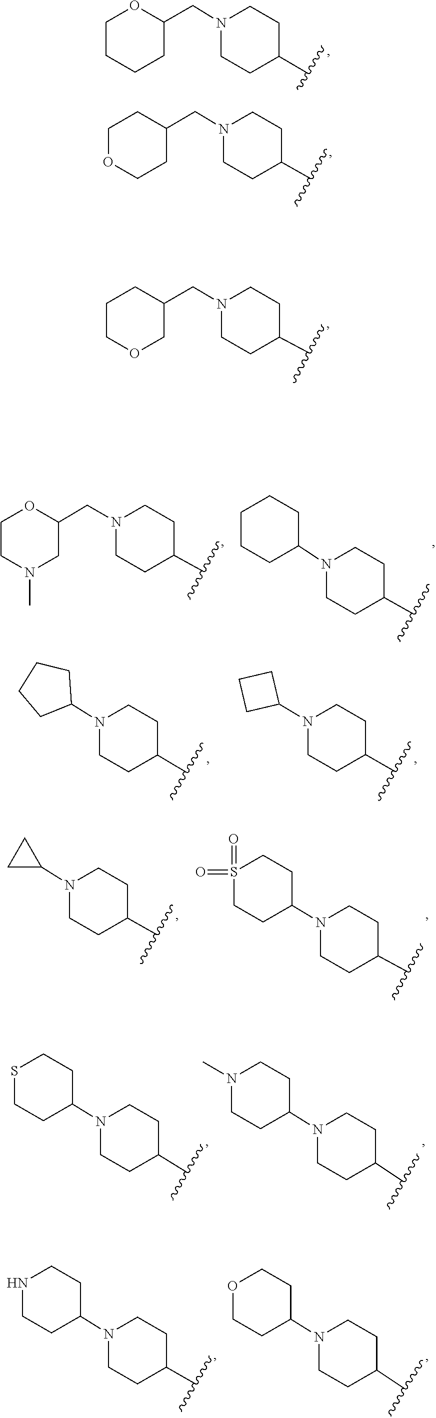

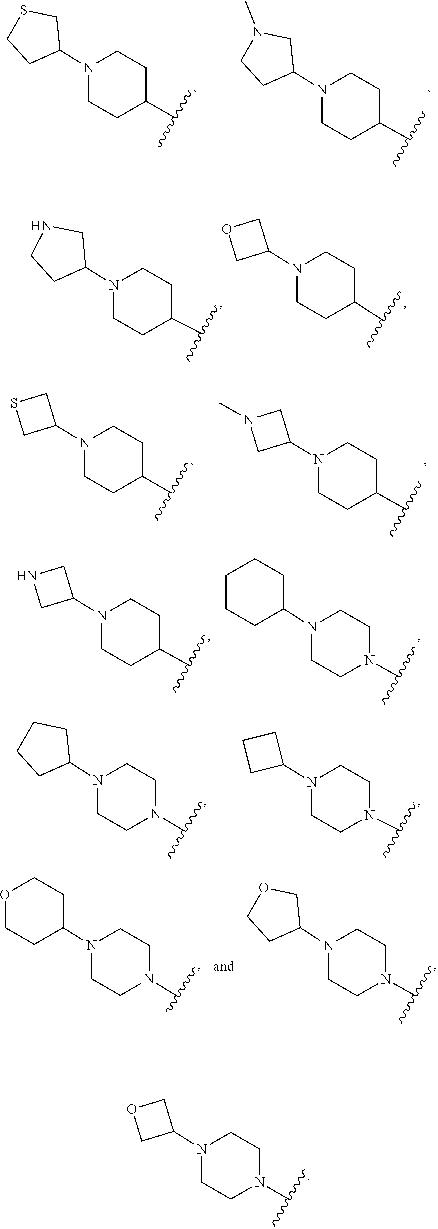

38. The compound of any one of claims 3 to 24, wherein A is selected from the group consisting

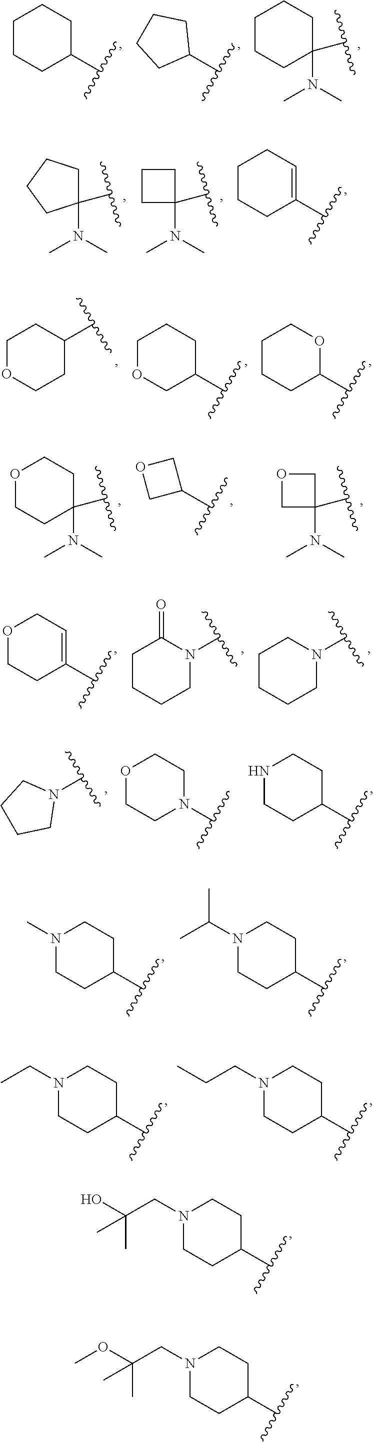

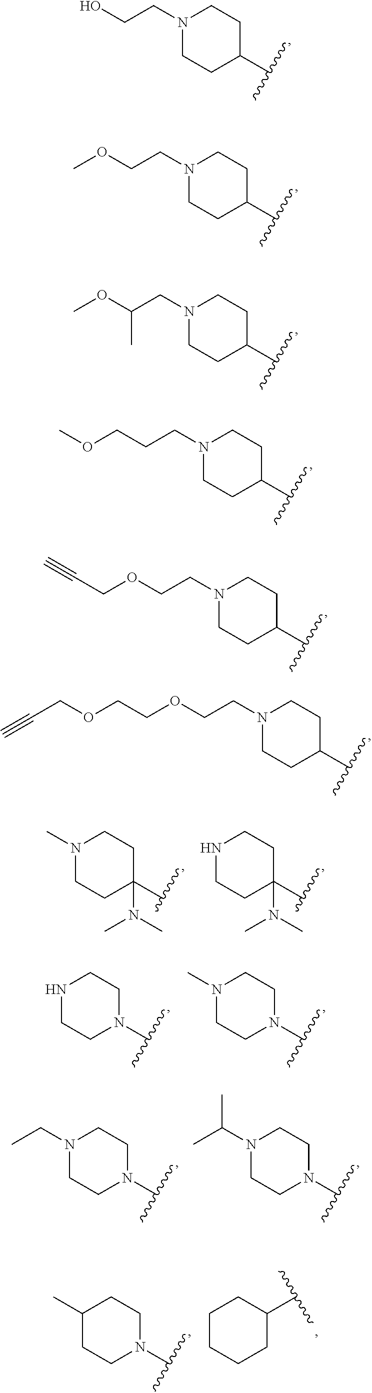

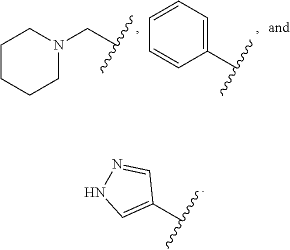

39. The compound of any one of claims 4 to 19, wherein A and R¹¹ are taken together with the atoms to which they are attached to form a C₃-C₇ cycloalkyl, aryl, 5-6 membered heteroaryl, or 3-7 membered heterocyclic ring, wherein the 5-6 membered heteroaryl and 3-7 membered heterocyclyl have 1-3 ring heteroatoms selected from N, O, and S, each of the rings optionally substituted by one R¹ group and optionally substituted by 1-4 independently selected R² groups.

40. The compound of any one of claims 3 to 19, wherein 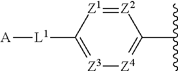
is selected from the group consisting of

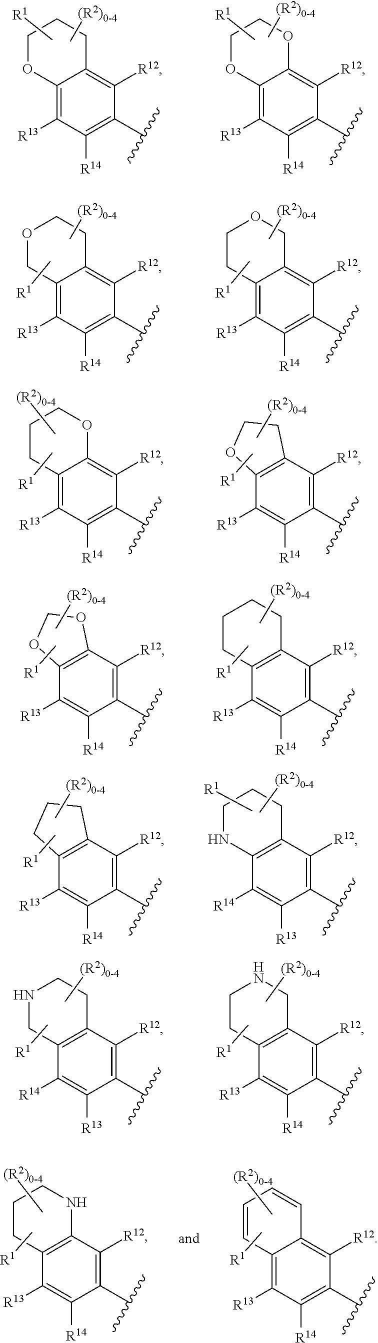

41. The compound of any one of claims 3 to 19, wherein 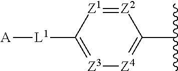
is selected from the group consisting of

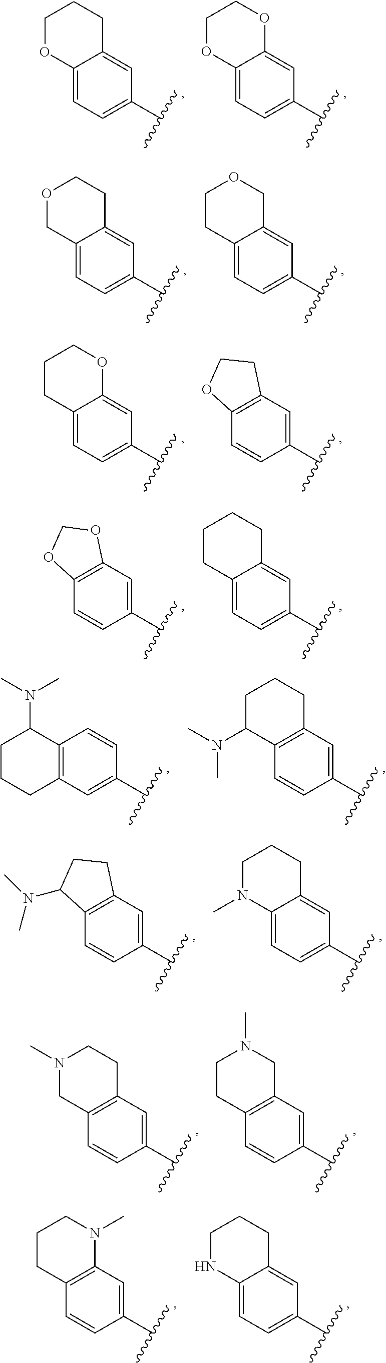

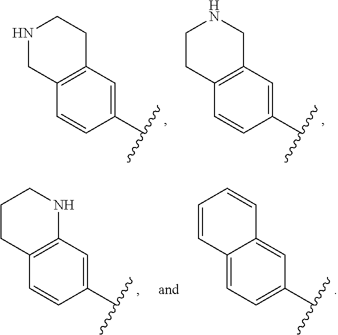 [SMILES: *c1ccc2c(c1)CCNC2 ; *c1ccc2c(c1)CNCC2 ; *c1ccc2c(c1)NCCC2 ; *c1ccc2ccccc2c1]

42. The compound of claim 3 or 4, wherein the compound is of Formula (IIa): 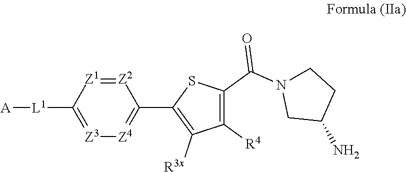
or stereoisomer and/or a pharmaceutically acceptable salt thereof.

43. The compound of claim 3 or 4, wherein the compound is of Formula (IIb): 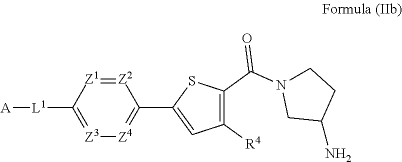
or stereoisomer and/or a pharmaceutically acceptable salt thereof.

44. The compound of claim 3 or 4, wherein the compound is of Formula (IIc): 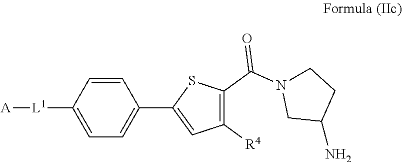
or stereoisomer and/or a pharmaceutically acceptable salt thereof.

45. The compound of claim 3 or 4, wherein the compound is of Formula (IId): 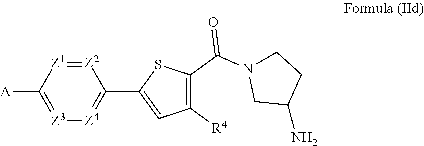
or stereoisomer and/or a pharmaceutically acceptable salt thereof.

46. A compound of Formula (III): 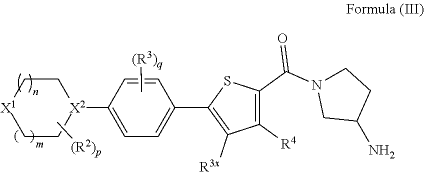
or stereoisomer and/or a pharmaceutically acceptable salt thereof, wherein:
X¹ is NR¹, O, S, SO₂, CH₂, or CHR¹;
X² is N or CH;
R¹ is selected from the group consisting of H, C₁-C₆ alkyl, C₂-C₆ alkenyl, C₂-C₆ alkynyl, C₁-C₆ heteroalkyl, C₁-C₆ haloalkyl, —NH₂, and -L²-G, wherein the C₁-C₆ alkyl may be optionally substituted with one or more OH, and wherein the C₁-C₆ heteroalkyl may be optionally substituted with C₂-C₆ heteroalkynyl;
L² is absent, or L² is selected from the group consisting of C₁-C₆ alkylene, C₁-C₆ heteroalkylene, —O—, —S—, and —NR′—, wherein the C₁-C₆ alkylene and C₁-C₆ heteroalkylene are optionally substituted by 1-4 substituents independently selected from the group consisting of ═O (oxo), OH, and halogen;
G is selected from the group consisting of C₃-C₇ cycloalkyl, 3-7 membered heterocycloalkyl, aryl, 5-6 membered heteroaryl, 6-10 membered carbocyclyl, and 5-10 membered heterocyclyl, wherein the 3-7 membered heterocycloalkyl, 5-6 membered heteroaryl, and 5-10 membered heterocyclyl have 1-3 ring heteroatoms selected from N, O, and S, and wherein G may be optionally substituted by 1-4 R^{A} substituents;
each R^{A} is independently selected from the group consisting of C₁-C₆ alkyl, C₁-C₆ heteroalkyl, C₁-C₆ haloalkyl, C₁-C₆ haloalkoxy, OH, CN, and halo;
each R² is independently selected from the group consisting of C₁-C₆ alkyl, C₂-C₆ alkenyl, C₂-C₆ alkynyl, C₁-C₆ heteroalkyl, C₁-C₆ alkoxy, C₁-C₆ haloalkyl, C₁-C₆ haloalkoxy, —NH₂, and halo;
each R³ is independently selected from the group consisting of C₁-C₆ alkyl, C₂-C₆ alkenyl, C₂-C₆ alkynyl, C₁-C₆ alkoxy, C₁-C₆ haloalkyl, C₁-C₆ haloalkoxy, OH, cyano, and halo;
R^{3x} is selected from the group consisting of H, C₁-C₆ alkyl, C₂-C₆ alkenyl, C₂-C₆ alkynyl, C₁-C₆ alkoxy, C₁-C₆ haloalkyl, cyano, and halo;
R⁴ is selected from the group consisting of H, C₁-C₆ alkyl, C₂-C₆ alkenyl, C₂-C₆ alkynyl, C₂-C₉ heteroalkynyl, C₁-C₆ heteroalkyl, C₁-C₆ haloalkyl, C₁-C₆ haloalkoxy, cyano, and halo;
each R′ is H or C₁-C₆ alkyl;
m is 0 or 1;
n is 0 or 1;
p is 0, 1, 2, 3, or 4; and
g is 0, 1, 2, 3, or 4.

47. A compound of Formula (III): 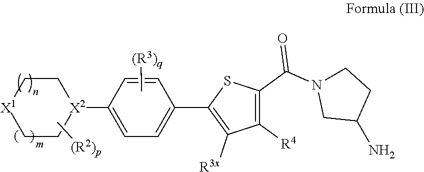
or stereoisomer and/or a pharmaceutically acceptable salt thereof, wherein:
X¹ is selected from the group consisting of NR¹, O, S, SO₂, CH₂, and CHR¹;
X² is N or CH;
R¹ is selected from the group consisting of H, C₁-C₆ alkyl, C₂-C₆ alkenyl, C₂-C₆ alkynyl, C₁-C₆ heteroalkyl, C₁-C₆ haloalkyl, —NH₂, and -G;
G is selected from the group consisting of C₃-C₇ cycloalkyl, 3-7 membered heterocycloalkyl, aryl, 5-6 membered heteroaryl, 6-10 membered carbocyclyl, and 5-10 membered heterocyclyl, wherein the 3-7 membered heterocycloalkyl, 5-6 membered heteroaryl, and 5-10 membered heterocyclyl have 1-3 ring heteroatoms selected from N, O, and S, and wherein G may be optionally substituted by 1-4 R^{A} substituents;
each R^{A} is independently selected from the group consisting of C₁-C₆ alkyl, C₁-C₆ heteroalkyl, C₁-C₆ haloalkyl, C₁-C₆ haloalkoxy, OH, CN, and halo;
each R² is independently selected from the group consisting of C₁-C₆ alkyl, C₁-C₆ haloalkyl, and halo;
each R³ is independently selected from the group consisting of C₁-C₆ alkyl, C₁-C₆ alkoxy, C₁-C₆ haloalkyl, cyano, and halo;
R^{3x} is selected from the group consisting of H, C₁-C₆ alkyl, C₂-C₆ alkenyl, C₂-C₆ alkynyl, C₁-C₆ alkoxy, C₁-C₆ haloalkyl, cyano, and halo;
R⁴ is selected from the group consisting of H, C₁-C₆ alkyl, C₁-C₆ alkoxy, C₁-C₆ haloalkyl, cyano, and halo;
m is 0 or 1;
n is 0 or 1;
p is 0, 1, 2, 3, or 4; and
q is 0, 1, 2, 3, or 4.

48. The compound of claim 47, wherein the compound is of Formula (IIIa): 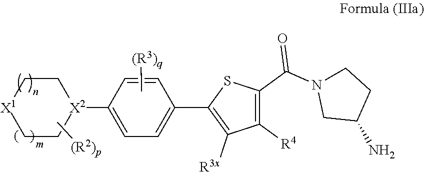
or stereoisomer and/or a pharmaceutically acceptable salt thereof.

49. The compound of any one of claims 46 to 48, wherein X¹ is NR¹ and X² is CH.

50. The compound of any one of claims 46 to 48, wherein X¹ is CH₂ or CHR¹ and X² is N.

51. The compound of any one of claims 46 to 48, wherein X¹ is NR¹ and X² is N.

52. The compound of any one of claims 46 to 51, wherein R¹ is selected from the group consisting of C₁-C₆ alkyl, C₁-C₆ alkoxy, C₁-C₆ haloalkyl, OH, and halo.

53. The compound of any one of claims 46 to 51, wherein R¹ is selected from the group consisting of -Me, -Et, -nPr, -iPr, —OMe, —OCF₃, —OH, —Cl, —F,

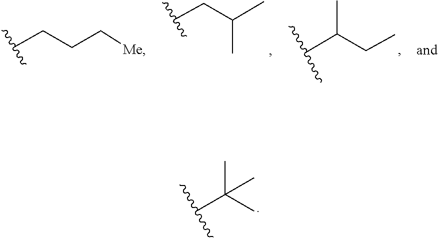

54. The compound of any one of claims 46 to 53, wherein R^{3x} is H or -Me.

55. The compound of any one of claims 46 to 53, wherein R^{3x} is H.

56. The compound of any one of claims 46 to 55, wherein R⁴ is -Me.

57. The compound of any one of claims 46 to 56, wherein m is 1 and n is 1.

58. The compound of any one of claims 46 to 56, wherein m is 0 and n is 1.

59. The compound of any one of claims 46 to 58, wherein p is 0.

60. The compound of any one of claims 46 to 59, wherein q is 0.

61. The compound of any one of claims 46 to 60, wherein the compound is of Formula (IIIb): 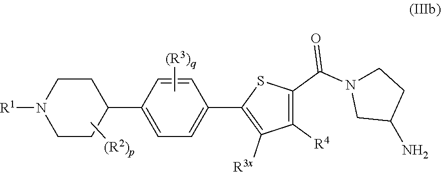
or stereoisomer and/or a pharmaceutically acceptable salt thereof.

62. The compound of any one of claims 46 to 60, wherein the compound is of Formula (IIIc): 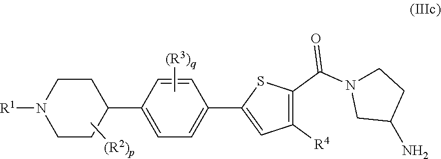
or stereoisomer and/or a pharmaceutically acceptable salt thereof.

63. The compound of any one of claims 46 to 60, wherein the compound is of Formula (IIId): 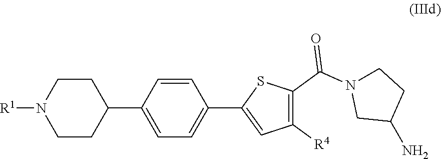
or stereoisomer and/or a pharmaceutically acceptable salt thereof

64. The compound of any one of claims 46 to 60, wherein the compound is of Formula (IIIe): 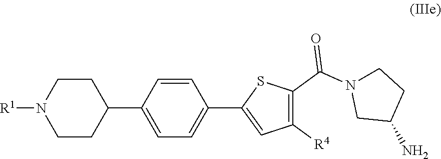
or a pharmaceutically acceptable salt thereof.

65. A compound of Formula (IV): 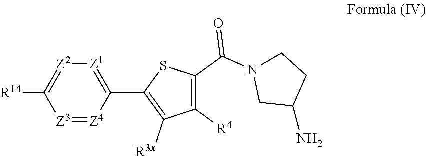
or stereoisomer and/or a pharmaceutically acceptable salt thereof, wherein:
Z¹ is N or CR¹¹;
Z² is N or CR¹²;
Z³ is N or CR¹³;
Z⁴ is N or CH;
R¹¹ is selected from the group consisting of H, —OH, halo, C₁-C₆ alkyl, C₁-C₆ heteroalkyl, and C₁-C₆ haloalkoxy;
R¹² is selected from the group consisting of H, —OH, halo, C₁-C₆ alkyl, C₁-C₆ heteroalkyl, and C₁-C₆ haloalkoxy;
R¹³ is H or halo;
R¹⁴ is selected from the group consisting of H, halo, C₁-C₆ alkylene, C₁-C₆ heteroalkylene, 5-7 membered aryl, 5-7 membered heteroaryl, and 3-7 membered heterocycloalkyl, wherein the R¹⁴ may be optionally substituted with one or more R^{14a};
R^{14a} is selected from the group consisting of C₁-C₆ alkyl, C₁-C₆ heteroalkyl, C₃-C₇ cycloalkyl, 5-7 membered aryl, 3-7 membered heterocycloalkyl, and —C(O)OR^{14b}, wherein the C₁-C₆ alkyl or the 3-7 membered heterocycloalkyl may be optionally substituted with one or more R^{14b};
R^{14b} is selected from the group consisting of —OH, oxo, C₁-C₆ alkyl, C₁-C₅ heteroalkyl, C₂-C₆ alkynyl, and 3-7 membered heterocycloalkyl, wherein the 3-7 membered heterocycloalkyl may be optionally substituted with one or more C₁-C₆ alkyl;
wherein when Z² is CR¹², R¹² and R¹⁴ may be taken together with the atoms to which they are attached to form a cycloalkyl, heterocycloalkyl, or aryl, wherein the cycloalkyl or the heterocycloalkyl may be optionally substituted with one or more C₁-C₆ alkyl or C₁-C₆ heteroalkyl;
R^{3x} is H or C₁-C₆ alkyl; and
R⁴ is selected from the group consisting of H, halo, C₁-C₆ alkyl, C₁-C₆ heteroalkyl, C₂-C₆ alkenyl, and C₁-C₆ haloalkoxy.

66. The compound of claim 65, wherein Z¹ is CR¹¹, Z² is CR¹², Z³ is CR¹³, and Z⁴ is CH.

67. The compound of claim 65, wherein when Z¹ is N, Z² is CR¹², Z³ is CR¹³, and Z⁴ is CH.

68. The compound of claim 65, wherein when Z² is N, Z¹ is CR¹¹, Z³ is CR¹³, and Z⁴ is CH.

69. The compound of claim 65, wherein when Z² and Z³ are N, Z¹ is CR¹¹ and Z⁴ is CH.

70. The compound of claim 65, wherein when Z² and Z⁴ are N, Z¹ is CR¹¹ and Z³ is CR¹³.

71. The compound of claim 65, wherein R¹¹ is selected from the group consisting of halo, C₁-C₆ alkyl, C₁-C₆ heteroalkyl, and C₁-C₆ haloalkoxy.

72. The compound of claim 65, wherein R¹² is selected from the group consisting of halo, C₁-C₆ alkyl, C₁-C₆ heteroalkyl, and C₁-C₆ haloalkoxy.

73. The compound of claim 71, wherein R¹¹ is selected from the group consisting of -Me, -Et, —Cl, —F, —OMe, and —OCF₃.

74. The compound of claim 71, wherein R¹² is selected from the group consisting of -Me, -Et, —Cl, —F, —OMe, and —OCF₃.

75. The compound of claim 71, wherein R¹¹ is —OH.

76. The compound of claim 71, wherein R¹² is —OH.

77. The compound of claim 65, wherein R¹³ is halo.

78. The compound of claim 77, wherein R¹³ is —Cl.

79. The compound of claim 65, wherein R¹⁴ is selected from the group consisting of H, halo, C₁-C₃ alkylene, C₁-C₆ heteroalkylene, phenyl, 5 membered heteroaryl, and 4-6 membered heterocycloalkyl, wherein the C₁-C₃ alkylene, C₁-C₆ heteroalkylene, phenyl, 5 membered heteroaryl, and 4-6 membered heterocycloalkyl may be optionally substituted with one or more R^{14a}.

80. The compound of claim 79, wherein R¹⁴ is selected from the group consisting of -Et, —O—, —CH₂—, -nPr, -iPr, —Cl, NHCH₃, —N(CH₃)₂, 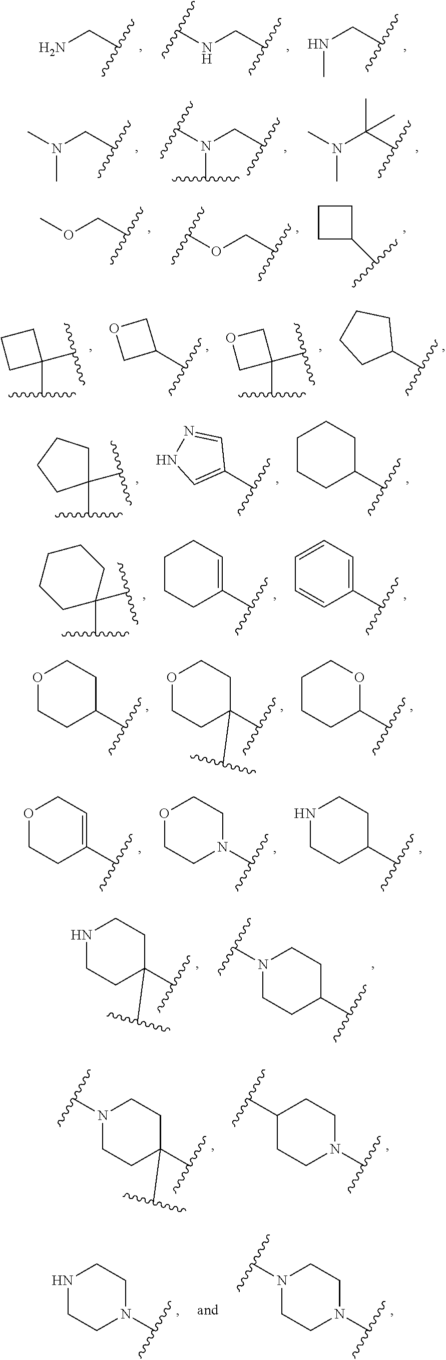
wherein if R¹⁴ contains a substitutable atom, that atom may be optionally substituted with one or more R^{14a}.

81. The compound of claim 80, wherein R¹⁴ is selected from the group consisting of -Et, -nPr, -iPr, —Cl, NHCH₃, —N(CH₃)₂,

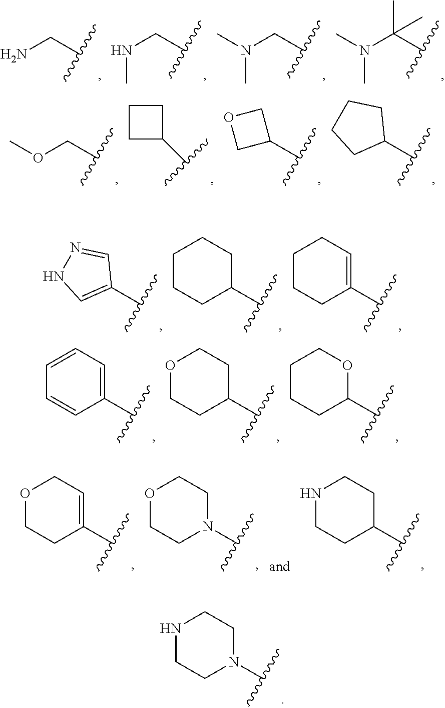

82. The compound of claim 79, wherein R¹⁴ is selected from the group consisting of

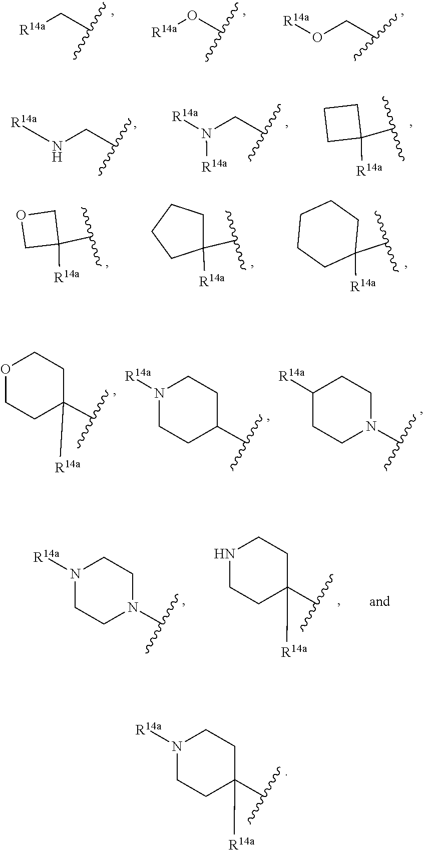

83. The compound of claim 79, 80, or 82, wherein R^{14a} is selected from the group consisting of C₁-C₃ alkyl, C₁-C₆ heteroalkyl, C₃-C₆ cycloalkyl, 6 membered aryl, and 4-6 membered heterocycloalkyl, wherein C₁-C₆ heteroalkyl, C₃-C₇ cycloalkyl, and 5-7 membered aryl may be optionally substituted with R^{14b}.

84. The compound of claim 83, wherein R^{14a} is selected from the group consisting of -Me, -Et, —CH₂—, nPr, 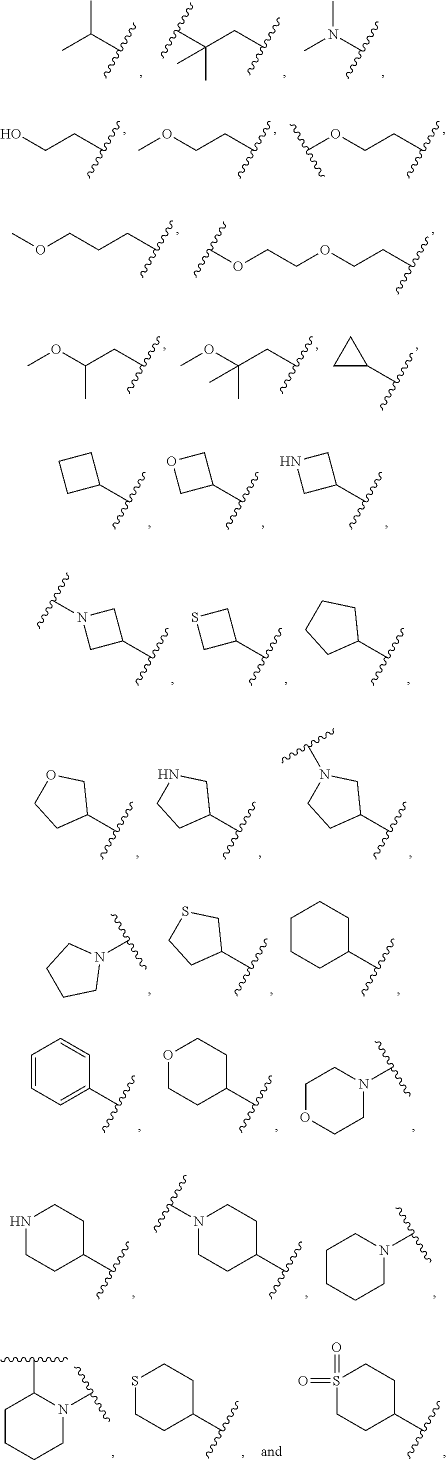
wherein if R^{14a} contains a substitutable atom, that atom may be optionally substituted with one or more R^{14b}.

85. The compound of claim 84, wherein R^{14a} is selected from the group consisting of -Me, -Et, nPr, 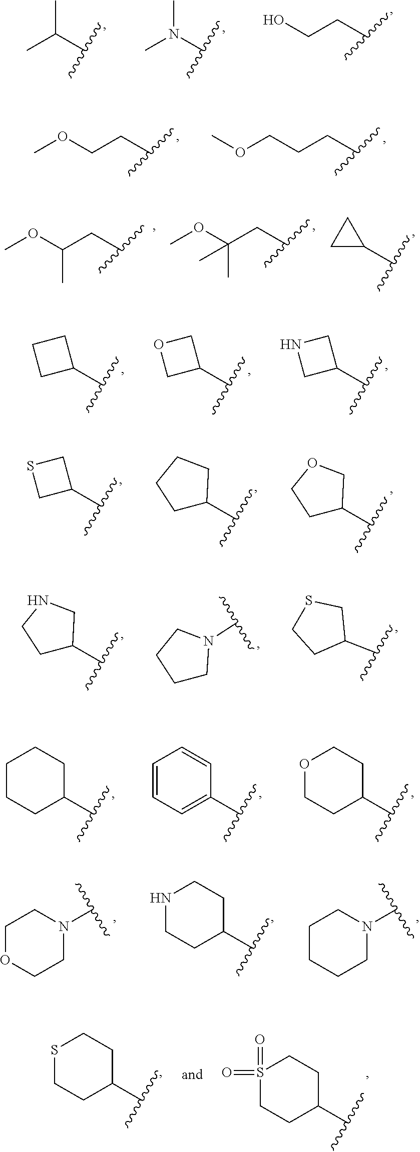
wherein if R^{14a} contains a substitutable atom, that atom may be optionally substituted with one or more R^{14b}.

85. The compound of claim 79, 82, or 83, wherein R^{14a} is selected from the group consisting of —C(O)OR^{14b},

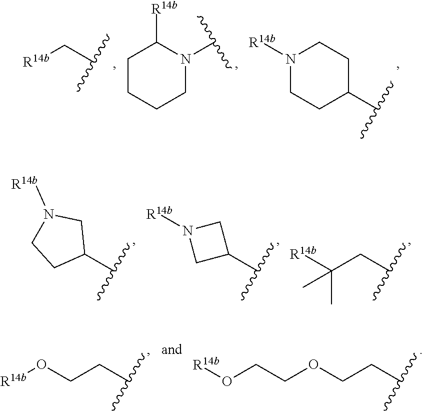

86. The compound of claim 79, 83, or 85, wherein R^{14b} is selected from the group consisting of oxo, —OH, -Me, C₃-C₇ heteroalkyl, C₂-C₃ alkynyl, and 6 membered heterocycloalkyl, wherein the 3-7 membered heterocycloalkyl may be optionally substituted with one or more C₁-C₆ alkyl.

87. The compound of claim 86, wherein R^{14b} is selected from the group consisting of oxo, —OH, -Me, 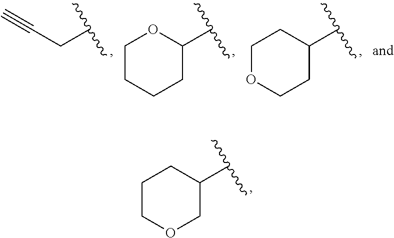
wherein the 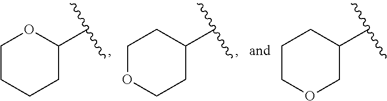
may be optionally substituted with oxo or C₁-C₆ alkyl.

88. The compound of claim 87, wherein R^{14b} is selected from the group consisting of oxo, —OH, -Me, 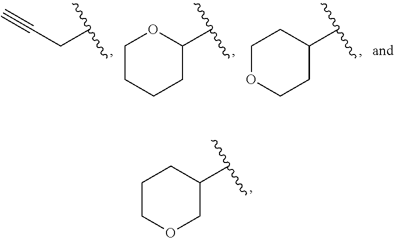
wherein the 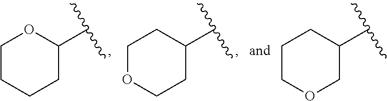
may be optionally substituted with -Me.

89. The compound of any one of claims 79-88, wherein when Z² is CR¹², R¹² and R¹⁴ may be taken together with the atoms to which they are attached to form an aryl, cycloalkyl, or heterocycloalkyl, wherein the cycloalkyl or heterocycloalkyl may be optionally substituted with one or more C₁-C₆ alkyl or C₁-C₆ heteroalkyl.

90. The compound of claim 89, wherein when Z² is CR¹², R¹² and R¹⁴ taken together are selected from the group consisting of,

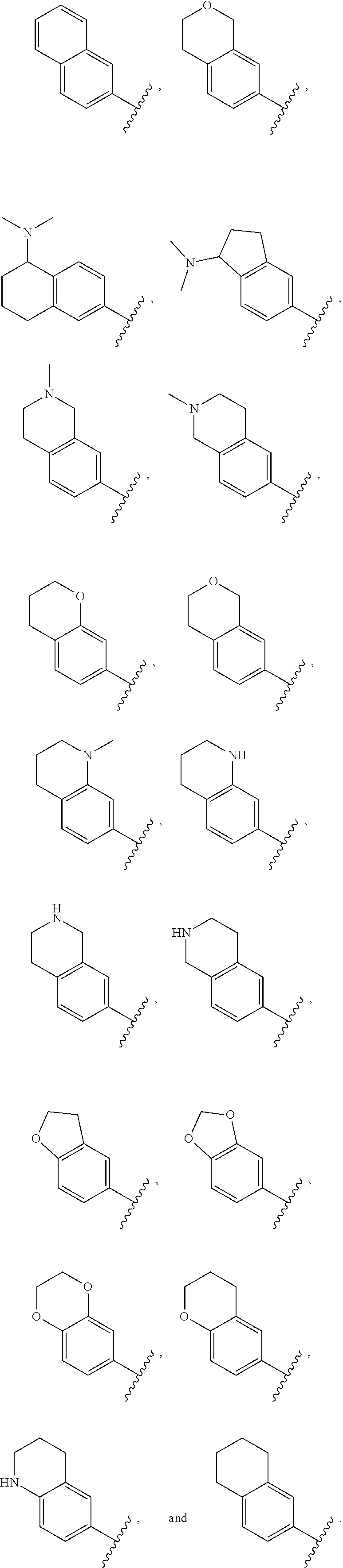

91. The compound of claim 65, wherein R^{3x} is C₁-C₆ alkyl.

92. The compound of claim 91, wherein R^{3x} is -Me.

93. The compound of claim 65, wherein R⁴ is selected from the group consisting of halo, C₁-C₆ alkyl, C₁-C₆ heteroalkyl, C₂-C₆ alkenyl, and C₁-C₆ haloalkoxy.

94. The compound of claim 93, wherein R⁴ is selected from the group consisting of —Cl, —Br, —OCF₃, -Me, -Et, —OMe,

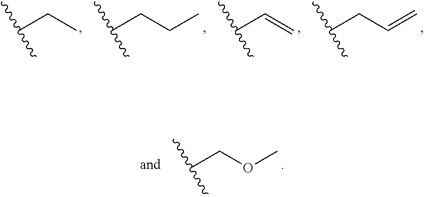

95. A compound selected from any compound set forth in Table 1, or stereoisomer and/or a pharmaceutically acceptable salt thereof.

96. A pharmaceutical composition comprising a pharmaceutically acceptable carrier and a compound of any one of claims 1 to 95.

97. A method of treating a subject with a neurodegenerative disease or disorder, wherein the method comprises administering to a subject in need thereof a pharmaceutically effective amount of a pharmaceutical composition of claim 96 or a compound of any one of claims 1 to 94.

98. The method of claim 97, wherein the neurodegenerative disease is selected from the group consisting of Alzheimer's disease, frontotemporal dementia (FTD), FTLD-U, FTD caused by mutations in the progranulin protein or tau protein (e.g., progranulin-deficient FTLD), frontotemporal dementia with inclusion body myopathy (IBMPFD), frontotemporal dementia with motor neuron disease, amyotrophic lateral sclerosis (ALS), Huntington's disease (HD), Huntington's chorea, prion diseases (e.g., Creutzfeld-Jacob disease, bovine spongiform encephalopathy, Kuru, or scrapie), Lewy Body disease, diffuse Lewy body disease (DLBD), polyglutamine (polyQ)-repeat diseases, trinucleotide repeat diseases, cerebral degenerative diseases, presenile dementia, senile dementia, Parkinsonism linked to chromosome 17 (FTDP-17), progressive supranuclear palsy (PSP), progressive bulbar palsy (PBP), pseudobulbar palsy, spinal and bulbar muscular atrophy (SBMA), primary lateral sclerosis, Pick's disease, primary progressive aphasia, corticobasal dementia, HIV-associated dementia, Parkinson's disease, Parkinson's disease with dementia, dementia with Lewy bodies, Down's syndrome, multiple system atrophy, spinal muscular atrophy (SMA, e.g., SMA Type I (e.g., Werdnig-Hoffnann disease) SMA Type II, SMA Type III (e.g., Kugelberg-Welander disease), or congenital SMA with arthrogryposis), progressive spinobulbar muscular atrophy (e.g., Kennedy disease), post-polio syndrome (PPS), spinocerebellar ataxia, pantothenate kinase-associated neurodegeneration (PANK), spinal degenerative disease/motor neuron degenerative diseases, upper motor neuron disorder, lower motor neuron disorder, age-related disorders and dementias, Hallervorden-Spatz syndrome, cerebral infarction, cerebral trauma, chronic traumatic encephalopathy, transient ischemic attack, Lytigo-bodig (amyotrophic lateral sclerosis-parkinsonism dementia), Guam-Parkinsonism dementia, hippocampal sclerosis, corticobasal degeneration, Alexander disease, Apler's disease, Krabbe's disease, neuroborreliosis, neurosyphilis, Sandhoff disease, Tay-Sachs disease, Schilder's disease, Batten disease, Cockayne syndrome, Kearns-Sayre syndrome, Gerstmann-Straussler-Scheinker syndrome and other transmissible spongiform encephalopathies, hereditary spastic paraparesis, Leigh's syndrome, demyelinating diseases, neuronal ceroid lipofuscinoses, epilepsy, tremors, depression, mania, anxiety and anxiety disorders, sleep disorders (e.g., narcolepsy, fatal familial insomnia), acute brain injuries (e.g., stroke, head injury), and autism, or any combination thereof.

99. The method of claim 97, wherein the neurodegenerative disease is amyotrophic lateral sclerosis (ALS).

100. The method of claim 97, wherein the neurodegenerative disease is Alzheimer's disease.
